(19) United States
(12) Patent Application Publication
(10) Pub. No.: US 2026/0356820 A1
(43) Pub. Date: Nov. 12, 2026

(54) Pyrrolidine and Imidazolidine Based DNA Polymerase Theta Inhibitors and Use Thereof

(71) Applicants: Thomas Jefferson University, Philadelphia, PA (US); Recombination Therapeutics, LLC, Doylestown, PA (US)

(72) Inventors: Richard T. Pomerantz, Philadelphia, PA (US); Mercy Ramanjulu, King of Prussia, PA (US)

(21) Appl. No.: 19/117,545

(22) PCT Filed: Oct. 3, 2023
(86) PCT No.: PCT/US2023/075798

§ 371 Date: Apr. 1, 2025

Related U.S. Application Data
(60) Provisional application No. 63/378,161, filed on Oct. 3, 2022.

(51) Int. Cl. C07D 403/06; A61K 31/402; A61K 31/4166; A61K 31/417; A61K 31/4178; A61K 31/4196; A61K 31/421; A61K 31/437; A61K 31/4439; A61K 31/454; A61K 31/496; A61K 31/5377; A61K 45/06; A61P 35/00; C07B 59/00; C07D 207/12; C07D 207/27; C07D 207/273; C07D 233/36; C07D 233/40; C07D 233/72; C07D 263/22; C07D 295/096; C07D 401/06; C07D 401/12; C07D 405/06; C07D 409/04; C07D 471/04

(52) U.S. Cl. CPC C07D 403/06; A61K 31/402; A61K 31/4166; A61K 31/417; A61K 31/4178; A61K 31/4196; A61K 31/421; A61K 31/437; A61K 31/4439; A61K 31/454; A61K 31/496; A61K 31/5377; A61K 45/06; A61P 35/00; C07B 59/002; C07D 207/12; C07D 207/27; C07D 207/273; C07D 233/36; C07D 233/40; C07D 233/72; C07D 263/22; C07D 295/096; C07D 401/06; C07D 401/12; C07D 405/06; C07D 409/04; C07D 471/04

(57) ABSTRACT

The invention relates to pyrrolidine and imidazolidine derivatives and their use in the treatment and prophylaxis of cancer, and to compositions containing said derivatives and processes for their preparation.

### CROSS-REFERENCE TO RELATED APPLICATIONS

This application claims the benefit of priority to U.S. Provisional Patent Application No. 63/378,161, filed Oct. 3, 2022, which is incorporated herein by reference in its entirety.

### STATEMENT REGARDING FEDERALLY SPONSORED RESEARCH OR DEVELOPMENT

This invention was made with government support under 1R41CA239983-01A1 and 1R41CA265430-01 awarded to Recombination Therapeutics, LLC by the National Cancer Institute and under W81XWH2010031 awarded by the Department of Defense. The government has certain rights in the invention.

### REFERENCE TO A “SEQUENCE LISTING” SUBMITTED AS AN XML FILE

The present application hereby incorporates by reference the entire contents of the XML file named “205961-0056-00WO_SequenceListing.xml” in XML format, which was created on Sep. 29, 2023, and is 10,036 bytes in size.

### FIELD OF INVENTION

The invention relates to pyrrolidine and imidazolidine derivatives and their use in the treatment and prophylaxis of cancer, and to compositions containing said derivatives and processes for their preparation.

### BACKGROUND OF THE INVENTION

Homology directed repair (HDR), also known as homologous recombination (HR), is an important DNA repair pathway due to its necessary role in promoting genome integrity and the completion of replication (Moynahan M E et al., 2010, Nat. Rev. Mol. Cell. Biol., 11:196-207; Li X et al., 2008, Cell Research, 18:99-113; Sung P et al., 2008, Nature Reviews. Molecular Cell Biology, 7:739-750). Mutations in proteins that play a central role HDR, such as BRCA1 or BRCA2 (BRCA), strongly predispose women to breast and ovarian cancer (Moynahan M E et al., 2010, Nat. Rev. Mol. Cell. Biol., 11:196-207). BRCA1 and BRCA2 are integral to HDR due to their critical roles in facilitating RAD51 loading onto single-strand DNA (ssDNA), which is essential for HDR repair of DNA breaks and hence cell proliferation (Moynahan M E et al., 2010, Nat. Rev. Mol. Cell. Biol., 11:196-207; Holloman W K, 2011, Nature Structural & Molecular Biology, 18:748-754; Lok B H et al., 2012, Clin. Cancer Res., 18:6400-6406; Lok B H et al., 2012, Oncogene). Since BRCA deficient cells are impaired in the HDR pathway of DNA repair, they are highly susceptible to DNA damage and therefore drugs that cause DNA damage or inhibit DNA repair can cause synthetic lethality in BRCA deficient cells while sparing normal cells (Farmer H et al., 2005, Nature, 434:917-921; Sonnenblick A et al., 2015, Nat. Rev. Clin. Oncol., 12:27-41; Lord C J et al., 2012, Nature, 481:287-294; Bryant H E et al., 2005, Nature, 434:913-917).

Developing small-molecules that target BRCA deficient cancers is highly significant for the development of precision medicine. The significance of developing drugs that target BRCA deficient cancers via the synthetic lethality approach has been exemplified by pioneering studies using Poly (ADP-ribose) polymerase 1 (PARP-1) inhibitors (Farmer H et al., 2005, Nature, 434:917-921; Lord C J et al., 2012, Nature, 481:287-294; Bryant H E et al., 2005, Nature, 434:913-917; Balmana J et al., 2011, Cancer Discov., 1:29-34). PARP-1 plays an important role in DNA base excision repair (BER) and cells that become deficient in BER due to PARP-1 inhibition undergo a high frequency of ssDNA breaks and PARP-1:DNA adducts which are converted to potentially lethal double-strand breaks (DSBs) during DNA replication. Since the BRCA proteins play a major role in repairing DSBs during S-phase, BRCA deficient cells are highly susceptible to DSBs and protein-DNA adducts caused by PARP-1 inhibitors as compared to normal cells. PARP-1 inhibitors have therefore shown promise in the clinic due to their ability to cause synthetic lethality in BRCA deficient cells (Sonnenblick A et al., 2015, Nat. Rev. Clin. Oncol., 12:27-41; Lord C J et al., 2017, Science, 355:1152-1158). These drugs, however, cause some side effects and patients frequently develop drug resistance, which indicates that the development of other anti-cancer drug targets is necessary (Sonnenblick A et al., 2015, Nat. Rev. Clin. Oncol., 12:27-41; Balmana J et al., 2011, Cancer Discov., 1:29-34; Edwards S L et al., 2008, Nature, 451:1111-1115; Sakai W et al., 2008, Nature, 451:1116-1120).

Previous studies identify DNA polymerase theta (referred to as Polq, Polθ, POLQ, or Pol-theta) as a promising drug target for BRCA deficient cancers (Mateos-Gomez P A et al., 2015, Nature, 518:254-257; Ceccaldi R L et al., 2015, Nature, 517). The polymerase domain encoded by gene POLQ, referred to herein as Polq, is a highly promiscuous enzyme. For example, unlike other polymerases, Polq promotes extension of single-strand DNA (ssDNA)(Hogg M et al., 2012, Nucleic Acids Res., 40:2611-2622; Kent T et al., 2015, Nature Structural & Molecular Biology, 22). Polq also exhibits low-fidelity DNA synthesis and translesion synthesis (TLS) activities (Hogg M et al., 2011, J. Mol. Biol., 405:642-652; Arana M E et al., 2008, Nucleic Acids Res., 36:3847-3856; Zahn K E et al., 2015, Nature Structural & Molecular Biology, 22:304-311). Studies have shown that human Polq promotes DSB repair via microhomology-mediated end-joining (MMEJ), also referred to as alternative end-joining (Mateos-Gomez P A et al., 2015, Nature, 518:254-257; Kent T et al., 2015, Nature Structural & Molecular Biology, 22; Kent T et al., 2015, Nat. Struct. Mol. Biol., 22:230-237; Black S J et al., 2019, Nat. Commun., 10:4423; Yousefzadeh M J et al., 2014, PLoS Genet., 10:e1004654). For example, Polq uniquely facilitates MMEJ of DNA with 3′ ssDNA overhangs containing microhomology tracts in vitro, and Polq was shown to promote MMEJ in mammalian cells (Mateos-Gomez P A et al., 2015, Nature, 518:254-257; Kent T et al., 2015, Nature Structural & Molecular Biology, 22; Yousefzadeh M J et al., 2014, PLoS Genet., 10:e1004654).

Although MMEJ is induced in response to DNA damage and replicative stress during S-phase, DSB repair is largely performed by HDR, which relies on BRCA1, BRCA2, and associated proteins, including, but not limited to RAD54, PALB2, RAD51C, FANCD2 (Moynahan M E et al., 2010, Nat. Rev. Mol. Cell. Biol., 11:196-207; Li X et al., 2008, Cell Research, 18:99-113; San Filippo J et al., 2008, Annual Review of Biochemistry, 77:229-257: Truong L N et al., 2013, Proc. Natl. Acad. Sci. USA, 110:7720-7725; Wang Z et al., 2019, J. Biol. Chem., 294:3909-3919). Since MMEJ serves as an important backup repair pathway, suppression of the expression of Polq in BRCA deficient cells causes synthetic lethality, but has little or no effect in BRCA proficient cells (Mateos-Gomez P A et al., 2015, Nature, 518:254-257; Cho N W et al., 2015, Nature, 518:174-176). For example, suppression of POLQ expression in cancer cells harboring mutations in BRCA1 results in a severe reduction in cell survival (Mateos-Gomez P A et al., 2015, Nature, 518:254-257). In contrast, suppression of POLQ in non-cancerous cells, for example those immortalized by expression of telomerase (hTERT), has no effect (Mateos-Gomez P A et al., 2015, Nature, 518:254-257). Consistent with this, BRCA deficient ovarian cancer cells were shown to be dependent on Polq for their survival in the presence of genotoxic agents (Ceccaldi R L et al., 2015, Nature, 517). This synthetic lethal relationship between Polq and HDR was further demonstrated in mouse models (Ceccaldi R L et al., 2015, Nature, 517). Most importantly, the DNA synthesis activity of Polq was shown to promote the survival of BRCA deficient cells (Mateos-Gomez P A et al., 2015, Nature, 518:254-257), which strongly suggests that pharmacological inhibition of the polymerase domain expressed by POLQ selectively kills BRCA deficient cancer cells.

More recent studies demonstrate that Polq is also synthetic lethal with many other DNA damage repair (DDR) factors. For example, it was shown that inactivation of Polq in combination with non-homologous end-joining factors such as Ku proteins is also synthetic lethal (Wyatt D W et al., 2016, Mol. Cell, 63:662-673; Feng W et al., 2019, Nat. Commun., 10:4286). Additionally, Polq inactivation in combination with DDR factors RAD54 or FANCJ also results in synthetic lethality (Feng W et al., 2019, Nat. Commun., 10:4286). Thus, overall Polq appears to play important roles in the survival of DNA repair defective cells, such as cells deficient in integral HDR or NHEJ factors. Furthermore, recent studies demonstrate that inactivation of Polq in combination with inhibition of the DDR factor ATR also results in a significant reduction in cell proliferation (Wang Z et al., 2019, J. Biol. Chem., 294:3909-3919). Semi-synthetic lethality between Polq and ATM was identified in earlier studies (Shima N et al., 2004, Mol. Cell Biol., 24:10381-10389). Thus, Polq is also an attractive drug target in cancers defective in the major DDR signaling pathways, including ATR, ATM and DNA-PKcs.

Thus, there is a need in the art for compositions and methods for inhibiting Polq for preventing or treating various diseases or disorders, such as cancer. The present invention satisfies this unmet need.

### SUMMARY OF THE INVENTION

In one aspect, the present invention relates to a compound having the structure of Formula (I), or a tautomeric or a stereochemically isomeric form, or a pharmaceutically acceptable salt or a solvate thereof:

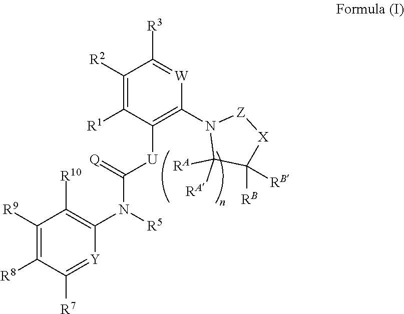

wherein:
U represents CH₂, O, S, or NR^{U};
W represents C(R⁴) or N;
Y represents C(R¹⁶) or N;
Q represents O or S;
R¹, R², R³, R⁴, R⁶, R⁷, R⁸, R⁹, and R¹⁰ independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, NR^{X}R^{Y}, and combinations thereof; wherein two adjacent groups R¹ to R⁴ or R⁶ to R¹⁰ optionally join to form a 5- to 7-membered saturated or unsaturated ring optionally containing one or more heteroatoms selected from O, N or S;
R⁵ and R^{U} independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof;
Z represents CR^{Z}R^{Z′}, C═S, or C═O;
X represents C(R¹⁵)(R¹⁶), N(R¹⁷) or O;
R¹⁵, R¹⁶, and R¹⁷ independently represent hydrogen, deuterium, C₁₋₆ alkyl, haloC₁₋₆ alkyl, —OR^{15a}, —SR^{15a}, nitrile, —COC₁₋₆ alkyl, —COOC₁₋₆ alkyl, hydroxy, C₁₋₆ alkoxy, C₁₋₆ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof; wherein R¹⁵, R¹⁶, and R¹⁷ may further comprise one or more divalent linkers L selected from the group consisting alkylene, cycloalkylene, heteroalkylene, heterocycloalkylene, alkenylene, alkynylene, arylene, heteroarylene, silyl, amine, amide, ester, ether, carbonyl, carbamate, sulfamate, sulfonic ester, sulfoximine, sulfonamide, thioether, thioester, disulfide, hydrazine, urea, thiourea, phosphate, phosphonate ester, poly(alkyl ether), heteroatom, and combinations thereof, wherein R¹⁵ and R¹⁶ may be taken together to form a ring;
n is 0, 1, or 2; wherein when n is 0, N is directly bonded to the carbon having R^{B} and R^{B′} bound thereto;
each R^{A}, R^{A′}, R^{Z}, and R^{Z′} independently represents hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof;
R^{B} and R^{B′} independently represents hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof, or R^{B} and the carbon to which it is bound together form a carbonyl group and R^{B′} is not present;
R^{15a} and R^{15b} independently represent hydrogen, deuterium, or C₁₋₆ alkyl; wherein two groups R^{15a} and R^{15b}, or two groups R^{15b}, may join together to form a 5 to 7 membered saturated ring system which may be optionally substituted by one or more C₁₋₆ alkyl groups;
R^{V}, R^{W}, R^{X} and R^{Y} independently represent hydrogen, deuterium, C₁₋₆ alkyl, haloC₁₋₆ alkyl, C₃₋₈ cycloalkyl, —COC₁₋₆ alkyl or heterocyclyl; wherein said alkyl groups may be optionally substituted with or more deuterium, hydroxy, amino or sulfone groups; and said heterocyclyl ring may be optionally substituted by one or more deuterium, oxo, hydroxy, C₁₋₆ alkanol or —COC₁₋₆ alkyl groups.

In one aspect, the present invention relates to a method of treating cancer in a subject in need thereof, the method comprising administering to the subject a compound of Formula (I) or a tautomeric or a stereochemically isomeric form, a pharmaceutically acceptable salt or a solvate thereof.

In one aspect, the present invention relates to a method of inhibiting the activity of DNA polymerase theta (Polq), the method comprising the step of contacting Polq with a compound of Formula (I) or a tautomeric or a stereochemically isomeric form, a pharmaceutically acceptable salt or a solvate thereof.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1F, depicts representative results from experiments showing that examples of Polq inhibitors (Polqi) selectively kill BRCA-mutant cancer cells. FIGS. 1A-1E depict scatter plots showing that the survival of BRCA2-mutant DLD-1 cells is significantly reduced by treatment with the indicated Polqi as compared to BRCA2-WT DLD-1 cells which are mostly resistant to Polqi. FIG. 1F depicts a scatter plot showing that the survival of BRCA2-mutant HCT116 cells is significantly reduced by treatment with the indicated Polqi as compared to BRCA2-WT HCT116 cells which are mostly resistant to Polqi.Data are represented as mean. n=3, +/−s.d.

FIG. 2 depicts representative results from experiments showing that Example 4 of a Polq inhibitor exhibits synergistic activity with the PARP inhibitor olaparib in reducing the survival of BRCA2-mutant cancer cells. A scatter plot is depicted showing the survival of BRCA2-mutant DLD1 cells in the presence of olapraib alone at the indicated concentrations compared to olaparib with two different concentrations of Polq inhibitor Example 4 (left). Data represent mean. n=3, +/−s.d. Synergy plot generated with ComBenefit software (right).

FIG. 3, comprising FIG. 3A through FIG. 3E, depicts representative experimental data demonstrating that Polq inhibitors act synergistically with PARP inhibitors. FIG. 3A depicts a representative scatter plot and synergy plot showing clongenic survival of HCT116 BRCA2-null cells treated with varying concentrations of olaparib with DMSO or various concentrations of Example 6. FIG. 3B depicts a representative scatter plot synergy plot showing clongenic survival of DLD1 BRCA2-null cells treated with varying concentrations of olaparib with DMSO or various concentrations of Example 6. FIG. 3C depicts a representative scatter plot synergy plot showing clongenic survival of PE01 cells treated with varying concentrations of olaparib with DMSO or various concentrations of Example 6. FIG. 3D depicts a representative scatter plot synergy plot showing clongenic survival of VC8 cells treated with varying concentrations of olaparib with DMSO or various concentrations of Example 6. FIG. 3E depicts a representative scatter plot synergy plot showing clongenic survival of CAPAN-1 cells treated with varying concentrations of olaparib with DMSO or various concentrations of Example 6. FIG. 3F depicts a representative scatter plot showing clongenic survival of MDA-MB-231 cells following treatment with various concentrations of talazoparib and DMSO or various concentrations of Example 6 and representative images of colony plates. For FIG. 3A through FIG. 3F, data are presented as mean±SEM; n=3; p-values calculated by two-sample t test. For FIG. 3A through FIG. 3E, *, p<0.05; **, p<0.01; ***, p<0.001. For FIG. 3F, at 50 nM talazoparib, p=0.019935 for DMSO vs Example 6.

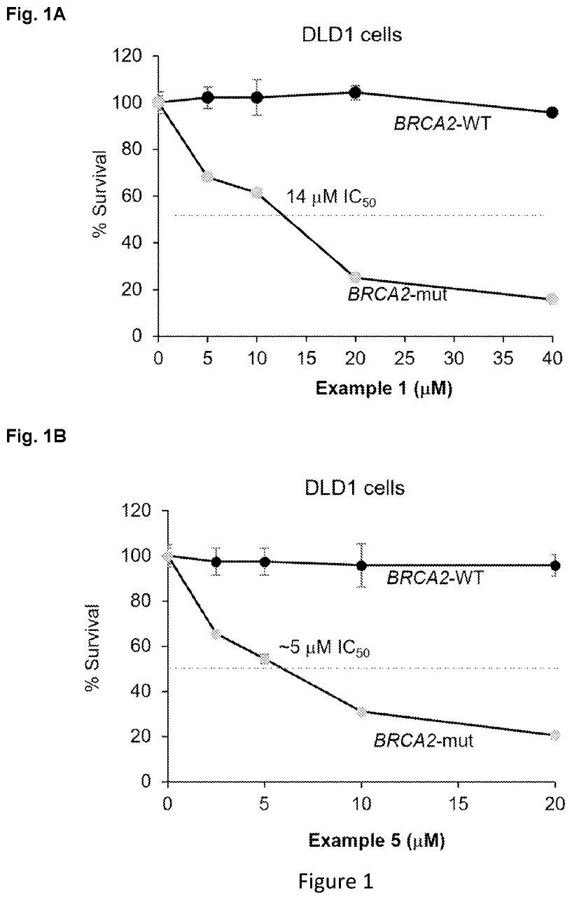

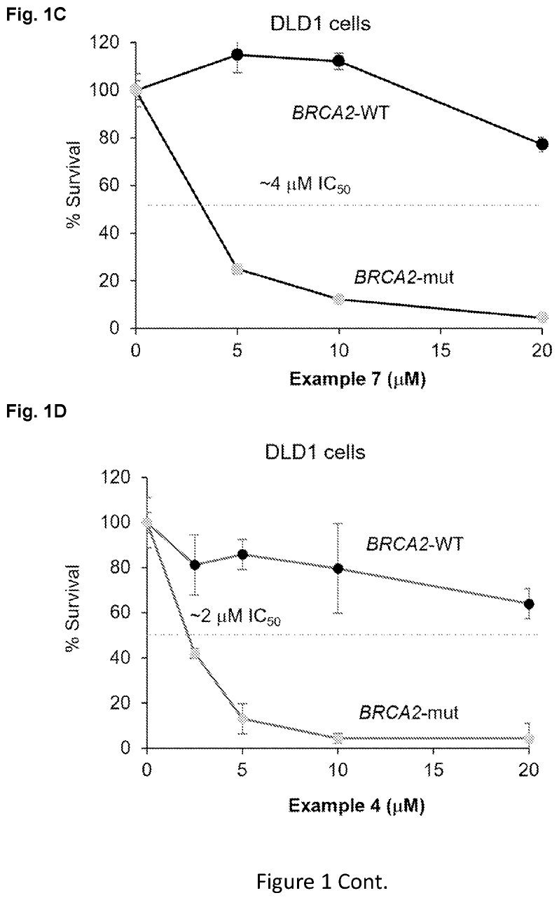

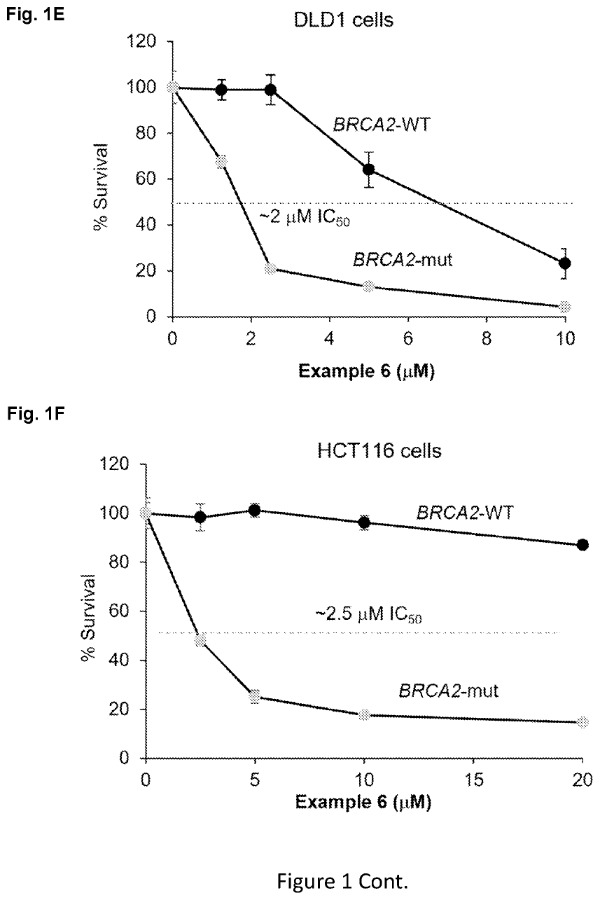

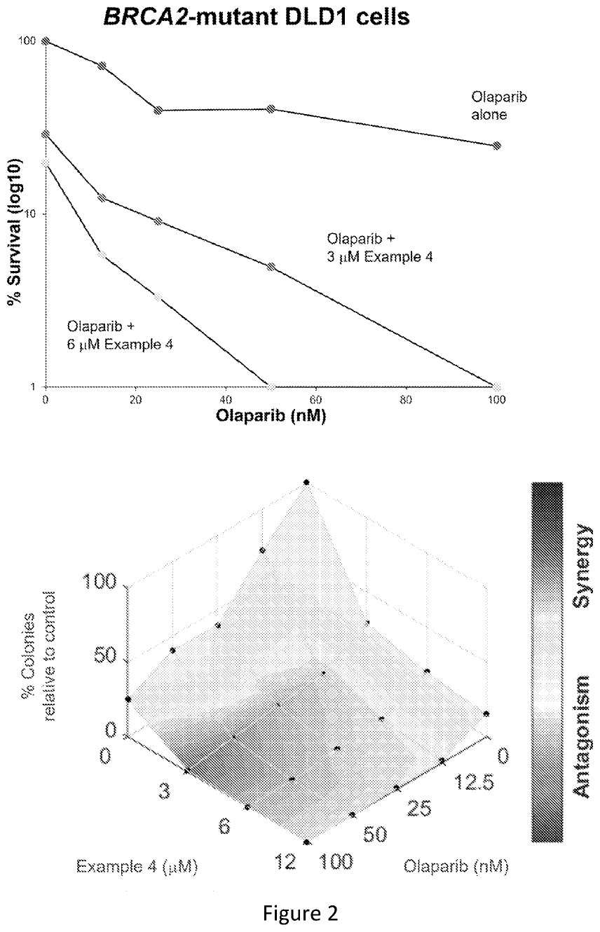

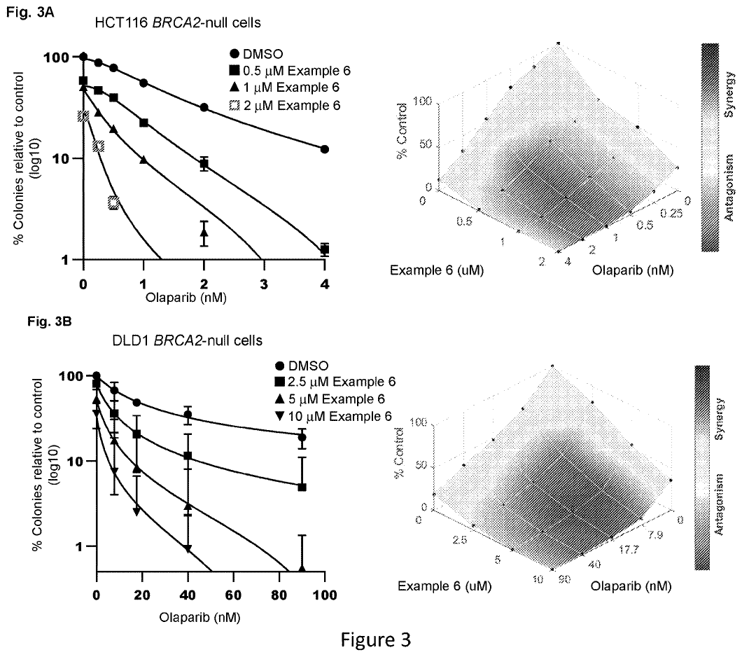

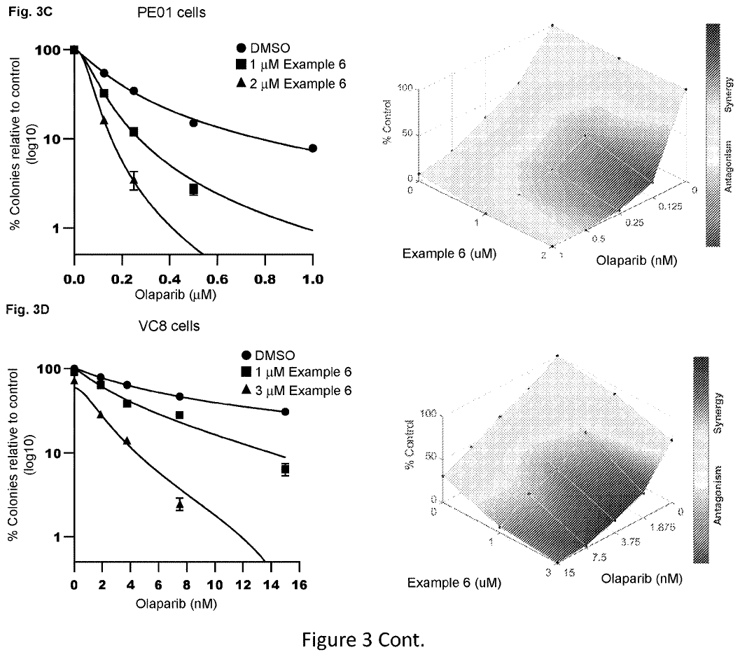

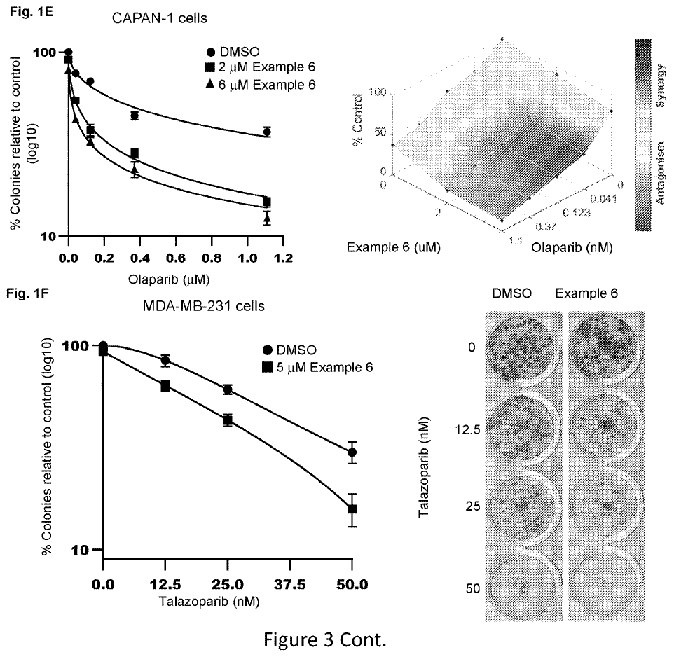

### DETAILED DESCRIPTION

The present invention is based, in part, on the discovery that novel pyrrolidine and imidazolidine based compounds inhibited Polq DNA synthesis activity. Thus, the present invention is directed, in part, to compositions comprising said pyrrolidine and imidazolidine based compounds and methods for inhibiting Polq in vitro and in vivo. In various embodiments, the Polq (e.g., the activity of Polq, the level of Polq, etc.) is essential for the proliferation of cancer cells, such as those defective in HDR or other DNA repair pathways. Thus, the present invention also provides, in part, compounds and methods for preventing or treating cancer with pyrrolidine and imidazolidine based compounds. The invention also provides a kit for modifying or inhibiting Polq (e.g., the activity of Polq, the level of Polq, etc.).

### DEFINITIONS

Unless defined otherwise, all technical and scientific terms used herein have the same meaning as commonly understood by one of ordinary skill in the art to which this invention belongs. As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “alkyl,” by itself or as part of another substituent means, unless otherwise stated, a straight or branched chain hydrocarbon having the number of carbon atoms designated (i.e., C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

The term “haloC₁₋₆ alkyl” as used herein as a group or part of a group refers to a C₁₋₆ alkyl group as defined herein wherein one or more than one hydrogen atom is replaced with a halogen. The term ‘haloC₁₋₆ alkyl’ therefore includes monohaloC₁₋₆ alkyl and also polyhaloC₁₋₆ alkyl. There may be one, two, three or more hydrogen atoms replaced with a halogen, so the haloC₁₋₆ alkyl may have one, two, three or more halogens. Examples of such groups include fluoroethyl, fluoromethyl, difluoromethyl, trifluoromethyl or trifluoroethyl and the like.

Similarly, the term “haloC₁₋₆ alkoxy” as used herein as a group or part of a group refers to a C₁₋₆ alkoxy group as defined herein wherein one or more than one hydrogen atom is replaced with a halogen. The term ‘haloC₁₋₆ alkoxy therefore includes monohaloC₁₋₆ alkoxy and also polyhaloC₁₋₆ alkoxy. There may be one, two, three or more hydrogen atoms replaced with a halogen, so the haloC₁₋₆ alkyl may have one, two, three or more halogens. Examples of such groups include fluoroethoxy, fluoromethoxy, difluoromethoxy, trifluoromethoxy or trifluoroethoxy and the like.

The term “C₃₋₈ cycloalkyl” as used herein refers to a saturated monocyclic hydrocarbon ring of 3 to 8 carbon atoms. Examples of such groups include cyclopropyl, cyclobutyl, cyclopentyl, cyclohexyl, cycloheptyl, cyclooctyl and the like.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkylene” by itself or as part of another molecule means a divalent radical derived from an alkane, as exemplified by (—CH₂—)ₙ. By way of example only, such groups include, but are not limited to, groups having 24 or fewer carbon atoms such as the structures —CH₂CH₂— and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense, and refer to alkyl groups linked to molecules via an oxygen atom, an amino group, a sulfur atom, respectively. As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, Si, P, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “aromatic” refers to a carbocycle or heterocycle with one or more polyunsaturated rings and having aromatic character, i.e., having (4n+2) delocalized □ (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to aryl groups which contain at least one heteroatom selected from N, O, Si, P, and S; wherein the nitrogen and sulfur atoms may be optionally oxidized, and the nitrogen atom(s) may be optionally quaternized. Heteroaryl groups may be substituted or unsubstituted. A heteroaryl group may be attached to the remainder of the molecule through a heteroatom. A polycyclic heteroaryl may include one or more rings that are partially saturated. Examples include tetrahydroquinoline, 2,3-dihydrobenzofuryl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. In another embodiment, the substituents are selected from the group consisting of hydrogen, deuterium, C₁₋₆ alkyl, C₂₋₆ alkenyl, hydroxy, C₁₋₆ alkoxy, halogen, haloC₁₋₆ alkyl, haloC₁₋₆ alkoxy, C₃₋₈ cycloalkyl, nitrile, amino, and combinations thereof. As used herein, where a substituent is an alkyl or alkoxy group, the carbon chain may be branched, straight or cyclic, with straight being preferred.

As used herein, “combinations thereof” refers to any combination of any two or more of the preceding substituents, without limit.

Several references to integers and R, R¹, R², R³, R⁴, R⁵, R⁶, etc. are made in chemical structures and moieties disclosed and described herein. Any description of integers and R, R¹, R², R³, R⁴, R⁵, R⁶, etc. in the specification is applicable to any structure or moiety reciting integers and R, R¹, R², R³, R⁴, R⁵, R⁶, etc. respectively.

As used herein, the term “protected,” as used herein, refers to the presence of a “protecting group” or moiety that prevents reaction of the chemically reactive functional group under certain reaction conditions. The protecting group will vary depending on the type of chemically reactive group being protected. By way of example only, (i) if the chemically reactive group is an amine or a hydrazide, the protecting group may be selected from tert-butyloxycarbonyl (t-Boc) and 9-fluorenylmethoxycarbonyl (Fmoc); (ii) if the chemically reactive group is a thiol, the protecting group may be orthopyridyldisulfide; and (iii) if the chemically reactive group is a carboxylic acid, such as butanoic or propionic acid, or a hydroxyl group, the protecting group may be benzyl or an alkyl group such as methyl, ethyl, or tert-butyl. Additionally, protecting groups include, but are not limited to, photolabile groups, such as Nvoc and MeNvoc, and other protecting groups known in the art. Other protecting groups are described in Greene and Wuts, Protective Groups in Organic Synthesis, 3rd Ed., John Wiley & Sons, New York, N.Y., 1999.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

“Pharmaceutically acceptable” refers to those properties and/or substances which are acceptable to the subject from a pharmacological/toxicological point of view and to the manufacturing pharmaceutical chemist from a physical/chemical point of view regarding composition, formulation, stability, subject acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

The term “pharmaceutically acceptable salt” refers to any pharmaceutically acceptable salt, which upon administration to the subject is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which the said compound is bonded by a non-covalent bond to another molecule (normally a polar solvent), including especially hydrates and alcoholates.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, topical, intraperitoneal, intramuscular, oral, aerosol, parenteral, ophthalmic, pulmonary, and topical administration.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, metabolites, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the compounds or compositions of the present invention. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

A “therapeutic” treatment is a treatment administered to a subject who exhibits signs or symptoms of a disease or disorder, for the purpose of diminishing or eliminating those signs or symptoms.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

As used herein, the terms “targeting domain”, “targeting moiety”, or “targeting group” are used interchangeably and refer to all molecules capable of specifically binding to a particular target molecule and forming a bound complex as described above. Thus, the ligand and its corresponding target molecule form a specific binding pair.

As used herein, the term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combination thereof.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human. In various embodiments, the subject is a human subject, and may be of any race, ethnicity, sex, and age.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “cancer” or “neoplasm” as used herein, include, but are not limited to, benign and malignant cancers of the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, melanoma, etc.), breast, genital system, (e.g., uterus, ovary, prostate, testis, etc.), urinary system (e.g, bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

As used herein, “treating a disease or disorder” means reducing the severity and/or frequency with which a sign or symptom of the disease or disorder is experienced by a subject.

A disease or disorder is “alleviated” if the severity of a sign or symptom of the disease or disorder, the frequency with which such a sign or symptom is experienced by a subject, or both, is reduced.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

By the term “modulating,” as used herein, is meant mediating a detectable increase or decrease in the level of a mRNA, polypeptide, or a response in a subject compared with the level of a mRNA, polypeptide or a response in the subject in the absence of a treatment or compound, and/or compared with the level of a mRNA, polypeptide, or a response in an otherwise identical but untreated subject. The term encompasses perturbing and/or affecting a native signal or response thereby mediating a beneficial therapeutic response in a subject, preferably, a human.

As used herein the terms “alteration,” “defect,” “variation,” or “mutation” refer to a mutation in a gene in a cell that affects the function, activity, expression (transcription or translation) or conformation of the polypeptide that it encodes. Mutations encompassed by the present invention can be any mutation of a gene in a cell that results in the enhancement or disruption of the function, activity, expression, or conformation of the encoded polypeptide, including the complete absence of expression of the encoded protein and can include, for example, missense and nonsense mutations, insertions, deletions, frameshifts, and premature terminations. Without being so limited, mutations encompassed by the present invention may alter splicing the mRNA (splice site mutation) or cause a shift in the reading frame (frameshift).

“Gene expression,” as used herein, encompasses the transcription of genomic DNA into mRNA and the translation of mRNA into protein.

A “genome” is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

“Encoding” refers to the inherent property of specific sequences of nucleotides in a polynucleotide, such as a gene, a cDNA, or an mRNA, to serve as templates for Synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Homologous, homology” or “identical, identity” as used herein, refer to comparisons among amino acid and nucleic acid sequences. When referring to nucleic acid molecules, “homology,” “identity,” or “percent identical” refers to the percent of the nucleotides of the subject nucleic acid sequence that have been matched to identical nucleotides by a sequence analysis program. Homology can be readily calculated by known methods. Nucleic acid sequences and amino acid sequences can be compared using computer programs that align the similar sequences of the nucleic or amino acids and thus define the differences. In preferred methodologies, the BLAST programs (NCBI) and parameters used therein are employed, and the ExPaSy is used to align sequence fragments of genomic DNA sequences. However, equivalent alignment assessments can be obtained through the use of any standard alignment software.

As used herein, “homologous” refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′ATTGCC 3′ and 5′TATGGC 3′ share 50% homology.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides.

In one embodiment, about 1500 nucleotides to about 2500 nucleotides.

In one embodiment, about 2500 nucleotides (and any integer value in between).

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

As used herein, the term “purified” or “to purify” refers to the removal of components (e.g., contaminants) from a sample. For example, nucleic acids are purified by removal of contaminating cellular proteins or other undesired nucleic acid species. The removal of contaminants results in an increase in the percentage of desired nucleic acid in the sample.

The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

As used herein, the term “nucleic acid” refers to both naturally-occurring molecules such as DNA and RNA, but also various derivatives and analogs. Generally, the probes, hairpin linkers, and target polynucleotides of the present teachings are nucleic acids, and typically comprise DNA. Additional derivatives and analogs can be employed as will be appreciated by one having ordinary skill in the art.

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6 delta 2-isopentenyladenine (6iA), N6-delta 2-isopentenyl-2-methylthioadenine (2 ms6iA), N2-dimethylguanine (dmG), 7methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, 6-methylguanine, N6-methyladenine, 04-methylthymine, 5,6-dihydrothymine, 5,6-dihydrouracil, pyrazolo(3,4-D)pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR2 or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-anomeric nucleotides, 1′-anomeric nucleotides, 2′-4′- and 3′-4′-linked and other “locked” or “LNA”, bicyclic sugar modifications (see, e.g., PCT published application nos. WO 98/22489, WO 98/39352; and WO 99/14226). The term “nucleic acid” typically refers to large polynucleotides.

The term “nucleotide analogs” as used herein refers to modified or non-naturally occurring nucleotides including, but not limited to, analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner and herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242; B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include nucleotides having one or more modification son the phosphate moiety, base moiety or sugar moiety, such as dideoxy nucleotides and 2′-O-methyl nucleotides. Nucleotide analogs include modified forms of deoxyribo-nucleotides as well as ribonucleotides.

Conventional notation is used herein to describe polynucleotide sequences: the left-hand end of a single-stranded polynucleotide sequence is the 5′-end. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.” In the sequences described herein:
A=adenine,
G=guanine,
T=thymine,
C=cytosine,
U=uracil,
H=A, C or T/U,
R=A or G,
M=A or C,
K=G or T/U,
S=G or C,
Y=C or T/U,
W=A or T/U,
B=G or C or T/U,
D=A or G, or T/U,
V=A or G or C,
N=A or G or C or T/U.

The skilled artisan will understand that all nucleic acid sequences set forth herein throughout in their forward orientation, are also useful in the compositions and methods of the invention in their reverse orientation, as well as in their forward and reverse complementary orientation, and are described herein as well as if they were explicitly set forth herein.

“Instructional material”, as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6, should be considered to have specifically disclosed subranges, such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates, in part, to the discovery that various novel pyrrolidine and imidazolidine derivatives selectively inhibited the polymerase domain of the POLQ gene product DNA polymerase theta (Polq). The present invention also relates, in part, to pharmaceutical formulations comprising said pyrrolidine and imidazolidine derivatives as well as methods of treating diseases or disorders, such as cancers defective in homology directed repair (HDR) (or homologous recombination), non-homologous end-joining, or other DNA damage response pathways by inhibiting Polq (e.g., the activity of Polq, the level of Polq, etc.) with disclosed pyrrolidine and imidazolidine derivatives and analogs thereof.

The present invention is based, in part, on the discovery that that novel pyrrolidine and imidazolidine based compounds presented herein inhibit Polq DNA synthesis activity. For example, pyrrolidine and imidazolidine based compounds containing aryl motifs flanking each end of an amide motif inhibit Polq DNA synthesis activity. Accordingly, the present invention provides methods and compositions for inhibiting Polq in vitro and in vivo. The present invention also demonstrates that pyrrolidine and imidazolidine based compounds and derivatives thereof that inhibit Polq activity additionally preferentially inhibit the proliferation of BRCA-deficient or HDR-deficient cancer cells. The present invention also demonstrates that pyrrolidine and imidazolidine based compounds and derivatives thereof that inhibit Polq activity additionally inhibit the proliferation of BRCA-deficient or HDR-deficient cancer cells in combination with PARP inhibitor (PARPi) treatment.

Polq is highly expressed in many types of cancer cells, confers resistance to ionizing radiation and various chemotherapy agents including etoposide, camptothecin and cisplatin, and promotes the survival of cancer cells, such as those deficient in HDR or other DNA repair or DDR pathways. High expression levels of Polq corresponds to a poor clinical outcome for cancer patients. Accordingly, another aspect of the invention provides a method of treating cancer in a subject by administering a composition of the invention.

In one embodiment, the method comprises administering a composition comprising a pyrrolidine or imidazolidine based compound, a pyrrolidine or imidazolidine based analog, a prodrug version of a pyrrolidine or imidazolidine based compound or derivative thereof, or a combination thereof. In some embodiments, the cancer is resistant to at least one type of chemotherapy agent. In some embodiments, the cancer is breast cancer. In some embodiments, the cancer is ovarian cancer. In some embodiments, the cancer is leukemia. In some embodiments, the cancer is prostate cancer. In some embodiments, the cancer is pancreatic cancer.

### DNA Polymerase Theta (Polq) Inhibitors

In one aspect, the present invention provides, in part, novel A family polymerase inhibitors. In one embodiment, the A family polymerase is DNA polymerase theta (Polq). In some embodiments, the A family polymerase is a fragment of Polq. In some embodiments, the fragment of Polq is Polq₁₇₉₂₋₂₅₉₀ (SEQ ID NO: 1) or a fragment thereof. In some embodiments, the fragment of Polq is a fragment of Polq₁₋₂₅₉₀ (SEQ ID NO: 2). In some embodiments, Polq is encoded by the human POLQ gene. In other embodiments, Polq is encoded by the Mus musculus Polq gene. In other embodiments, Polq is encoded by the C. elegans polq-1 gene.

Thus, in various embodiments, the present invention provides compounds that modulate or inhibit the level or activity of at least one A family polymerase (e.g., Polq). In another aspect, the present invention provides compounds useful for preventing or treating a disease or disorder (e.g., cancer). In various aspects, the compound of the present invention is a functionalized pyrrolidine or imidazolidine based compound or derivative thereof.

### Compounds

In one aspect, the present invention relates to a functionalized pyrrolidine or imidazolidine derivative such as compounds having the structure of Formula (I), or a tautomeric or a stereochemically isomeric form, a pharmaceutically acceptable salt or a solvate thereof:

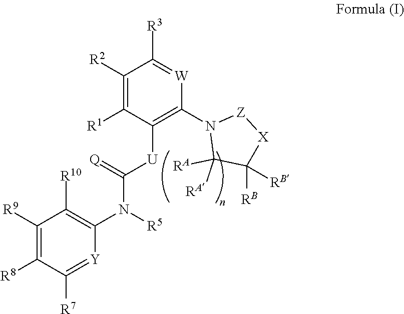

wherein:
U represents CH₂, O, S, or NR^{U};
W represents C(R⁴) or N;
Y represents C(R¹⁶) or N;
Q represents O or S;
R¹, R², R³, R⁴, R⁶, R⁷, R⁸, R⁹, and R¹⁰ independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, NR^{X}R^{Y}, and combinations thereof; wherein two adjacent groups R¹ to R⁴ or R⁶ to R¹⁰ optionally join to form a 5- to 7-membered saturated or unsaturated ring optionally containing one or more heteroatoms selected from O, N or S;
R⁵ and R^{U} independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof;
Z represents CR^{Z}R^{Z′}, C═S, or C═O;
X represents C(R¹⁵)(R¹⁶), N(R¹⁷) or O;
R¹⁵, R¹⁶, and R¹⁷ independently represent hydrogen, deuterium, C₁₋₆ alkyl, haloC₁₋₆ alkyl, —OR^{15a}, —SR^{15a}, nitrile, —COC₁₋₆ alkyl, —COOC₁₋₆ alkyl, hydroxy, C₁₋₆ alkoxy, C₁₋₆ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof; wherein R¹⁵, R¹⁶, and R¹⁷ may further comprise one or more divalent linkers L selected from the group consisting alkylene, cycloalkylene, heteroalkylene, heterocycloalkylene, alkenylene, alkynylene, arylene, heteroarylene, silyl, amine, amide, ester, ether, carbonyl, carbamate, sulfamate, sulfonic ester, sulfoximine, sulfonamide, thioether, thioester, disulfide, hydrazine, urea, thiourea, phosphate, phosphonate ester, poly(alkyl ether), heteroatom, and combinations thereof; wherein R¹⁵ and R¹⁶ may be taken together to form a ring;
n is 0, 1, or 2; wherein when n is 0, N is directly bonded to the carbon having R^{B} and R^{B′} bound thereto;
each R^{A}, R^{A′}, R^{Z}, and R^{Z′} independently represents hydrogen, deuterium, C₁₋₆ alkyl, hydroxy, C₁₋₆ alkoxy, C₁₋₆ alkanol, halogen, —O^{15b}, CO₂H, CO₂R^{15b}, haloC₁₋₆ alkyl, and combinations thereof;
R^{B} and R^{B′} independently represents hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof; or R^{B} and the carbon to which it is bound together form a carbonyl group and R^{B′} is not present;
R^{15a} and R^{15b} independently represent hydrogen, deuterium, or C₁₋₆ alkyl; wherein two groups R^{15a} and R^{15b}, or two groups R^{15b}, may join together to form a 5 to 7 membered saturated ring system which may be optionally substituted by one or more C₁₋₆ alkyl groups;
R^{V}, R^{W}, R^{X} and R^{Y} independently represent hydrogen, deuterium, C₁₋₆ alkyl, haloC₁₋₆ alkyl, C₃₋₈ cycloalkyl, —COC₁₋₆ alkyl or heterocyclyl; wherein said alkyl groups may be optionally substituted with or more deuterium, hydroxy, amino or sulfone groups; and said heterocyclyl ring may be optionally substituted by one or more deuterium, oxo, hydroxy, C₁₋₆ alkanol or —COC₁₋₆ alkyl groups.

In one embodiment, the compound of Formula (I) is represented by Formula (Ia):

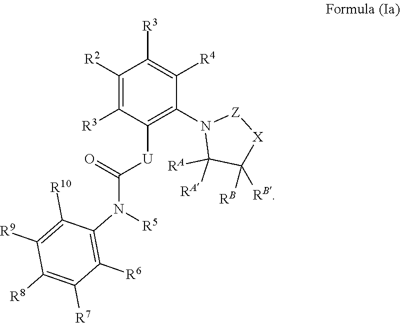

In one embodiment, the compound of Formula (I) is represented by Formula (Ib):

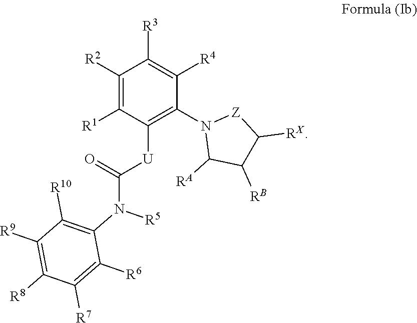

In one embodiment, the compound of Formula (I) is represented by Formula (Ic) or Formula (Ic′):

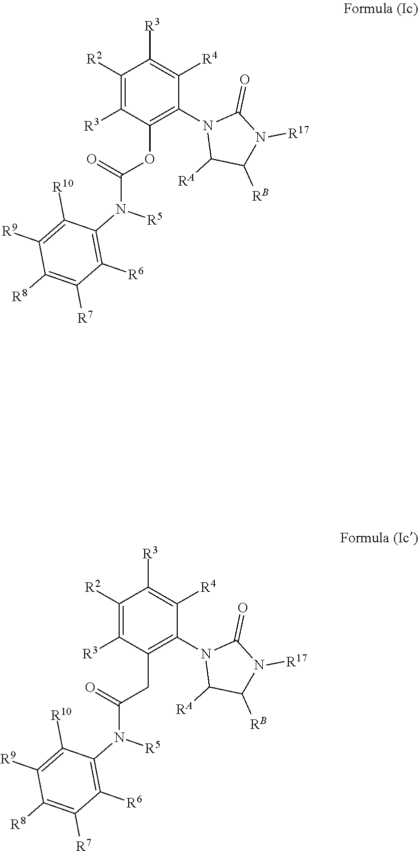

In one embodiment, the compound of Formula (I) is represented by Formula (Id) or Formula (Id′):

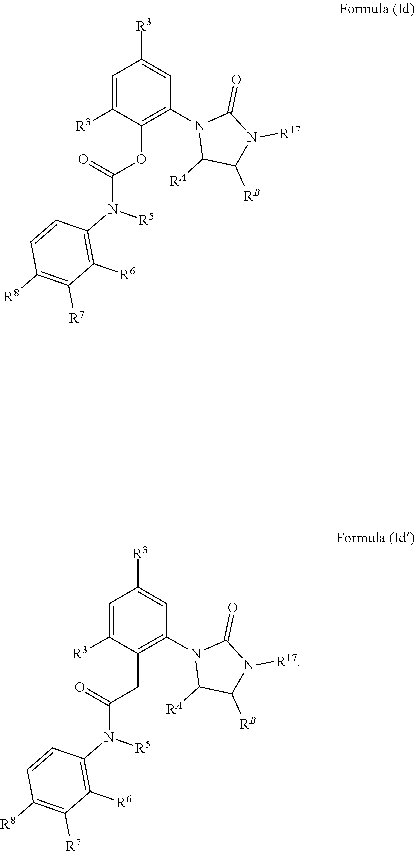

In one embodiment, the compound of Formula (I) is represented by Formula (Ie) or Formula (Ie′):

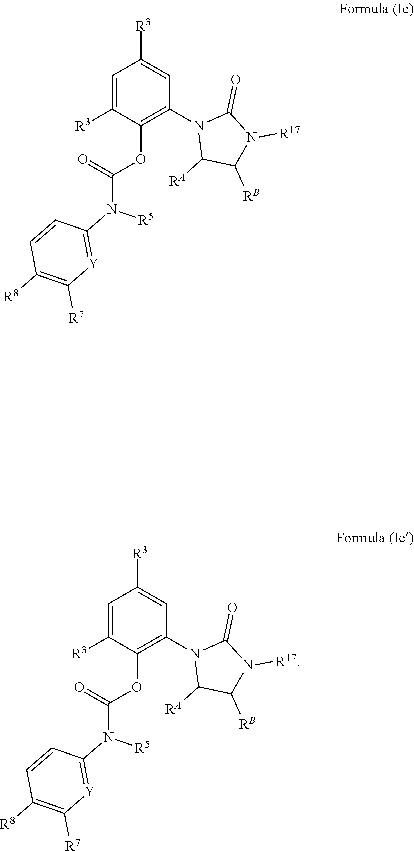

In one embodiment, U is O. In one embodiment, U is CH₂. In one embodiment, U is S. In one embodiment, Q is O. In one embodiment, Q is S.

In one embodiment, R⁷ and R⁸ or R⁸ and R⁹ join to form a pyrrolyl ring which is optionally substituted. In one embodiment, Y is N. In one embodiment, R^{A} and R^{A′} each represent hydrogen or deuterium. In one embodiment, Z represents C═O. In one embodiment, Z represents C═S. In one embodiment, Z represents CR^{Z}R^{Z′}. In one embodiment, R^{Z} and R^{Z′} each represent hydrogen or deuterium.

In one embodiment, R¹⁷ represents hydrogen, deuterium, C₁₋₆ alkyl, haloC₁₋₆ alkyl, —OR^{15a}, —SR^{15a}, nitrile, —COC₁₋₆ alkyl, —COOC₁₋₆ alkyl, hydroxy, C₁₋₆ alkoxy, C₁₋₆ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof. In one embodiment, R¹⁷ further comprises a divalent linker selected from the group consisting alkylene, cycloalkylene, heteroalkylene, heterocycloalkylene, alkenylene, alkynylene, arylene, heteroarylene, silyl, amine, amide, ester, ether, carbonyl, carbamate, sulfamate, thioether, thioester, disulfide, hydrazine, urea, thiourea, phosphate, phosphonate ester, poly(alkyl ether), heteroatom, and combinations thereof. In one embodiment, R¹⁷ comprises one or more divalent linkers. In one embodiment, R¹⁷ has the structure —CH₂CH₂O-L-R^{17′}, wherein R^{17′} represents hydrogen, deuterium, C₁₋₆ alkyl, haloC₁₋₆ alkyl, —OR^{15a}, —SR^{15a}, nitrile, —COC₁₋₆ alkyl, —COOC₁₋₆ alkyl, hydroxy, C₁₋₆ alkoxy, C₁₋₆ alkanol, C₃₋₈ cycloalkyl, halogen, carbonyl, —NR^{V}R^{W}, —CH₂—NR^{V}R^{W}, —OSO₂NH₂, aryl, heteroaryl, heterocyclyl, and combinations thereof.

In one embodiment, R¹⁷ represents a substituent selected from the following:

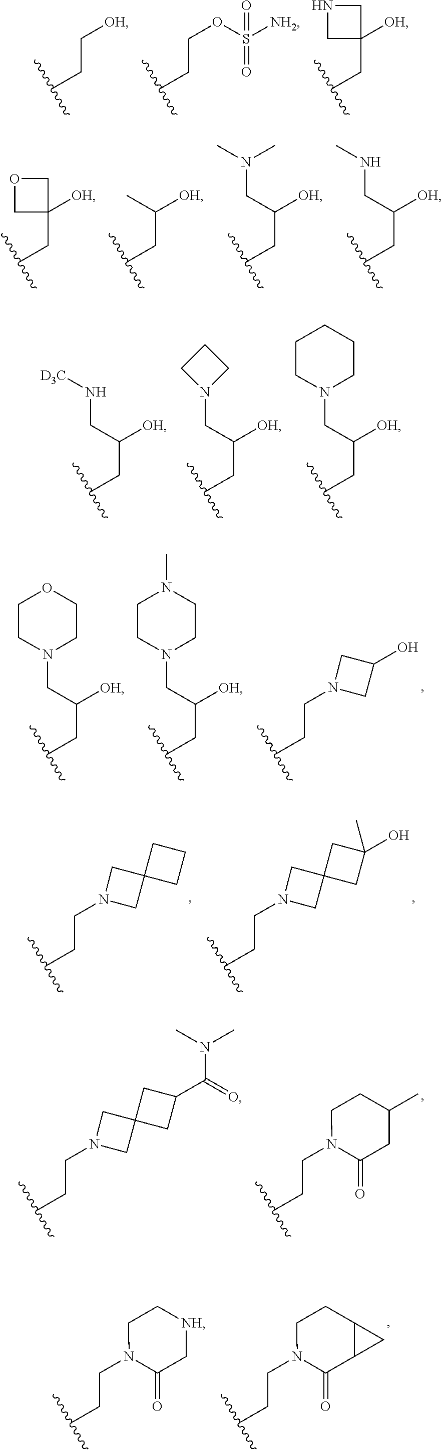

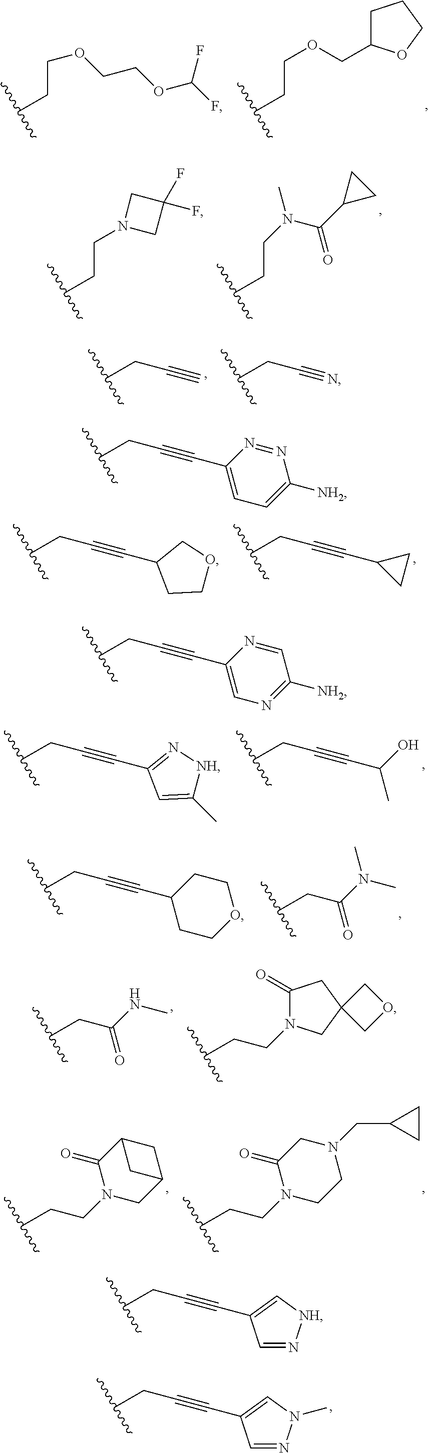

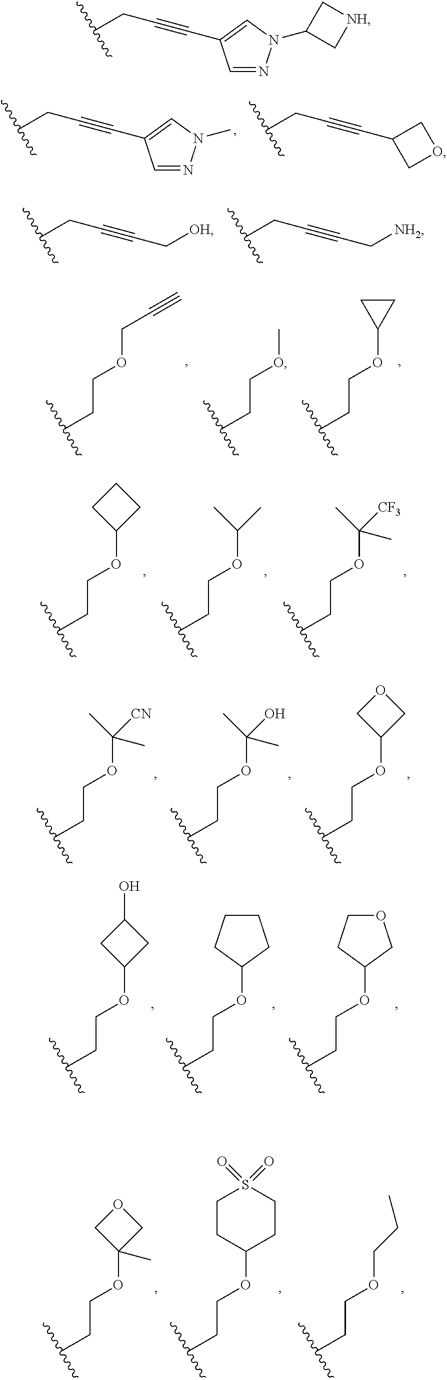

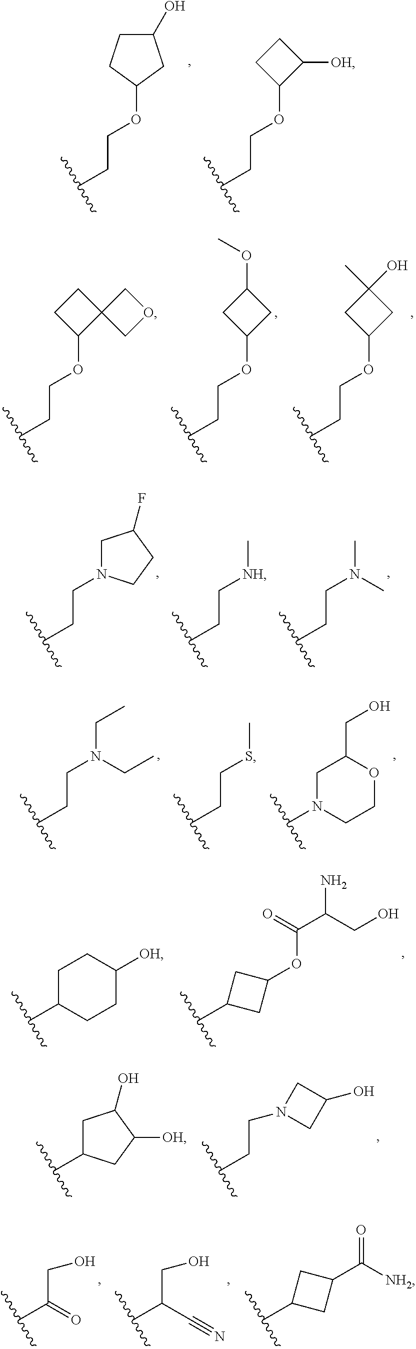

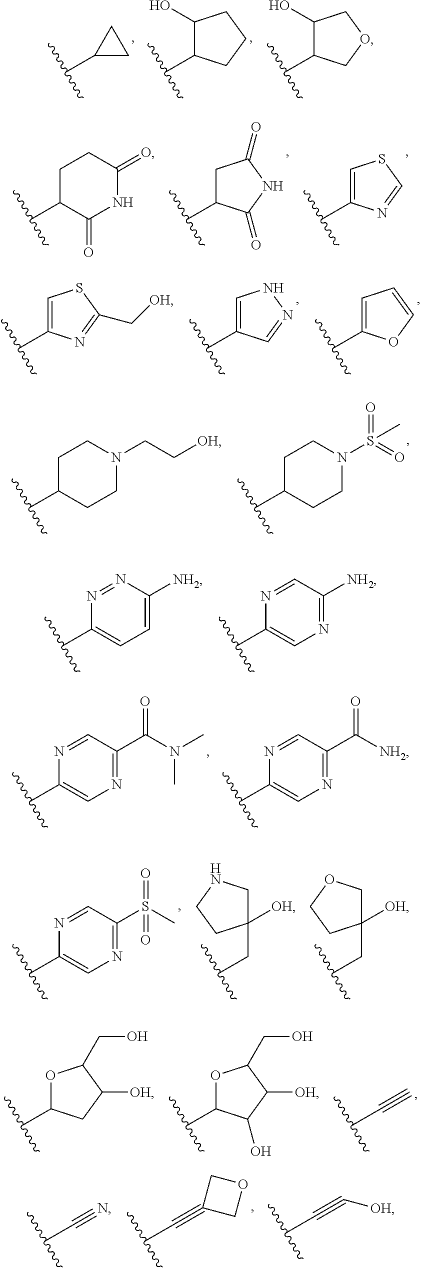

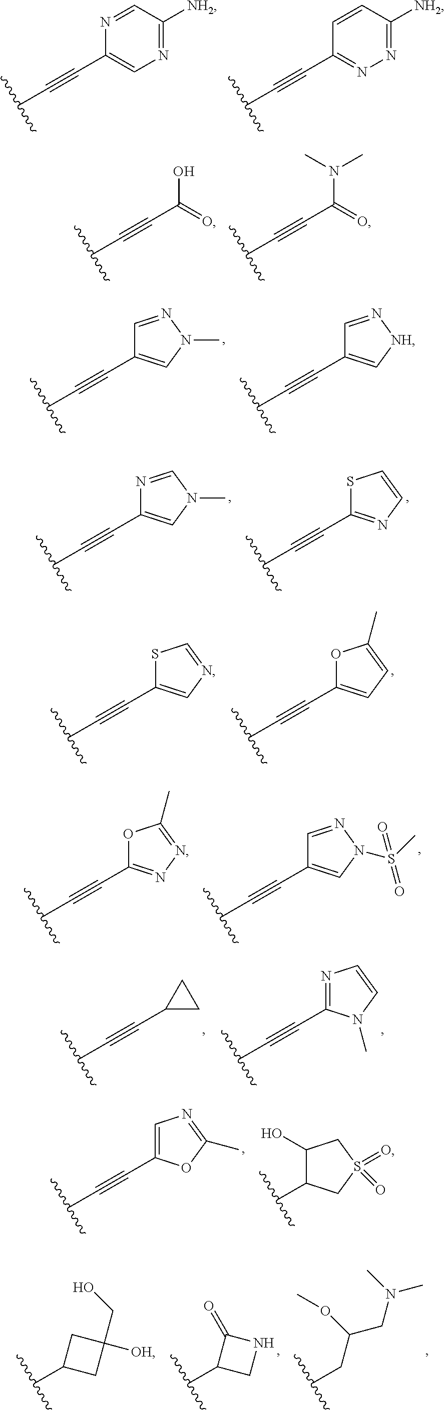

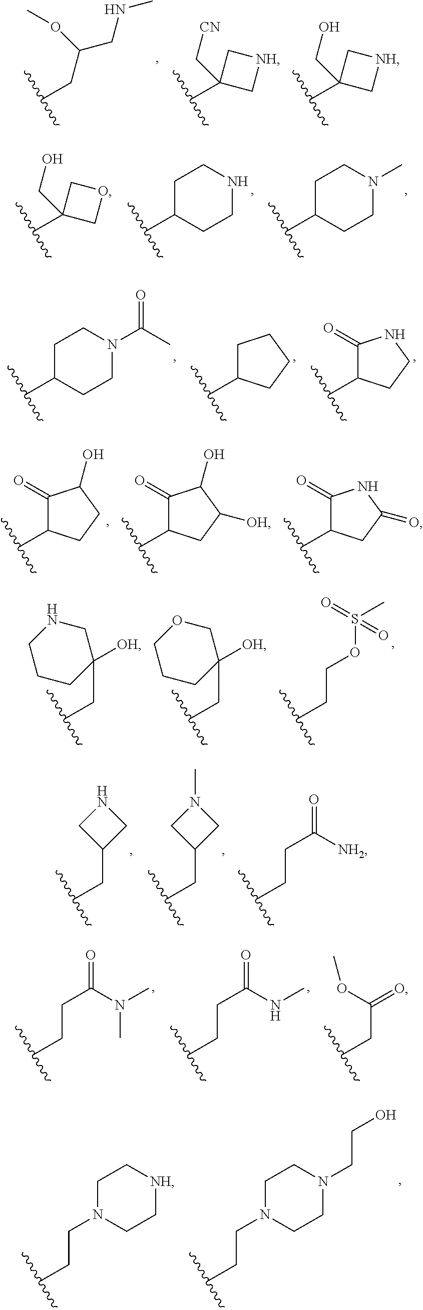

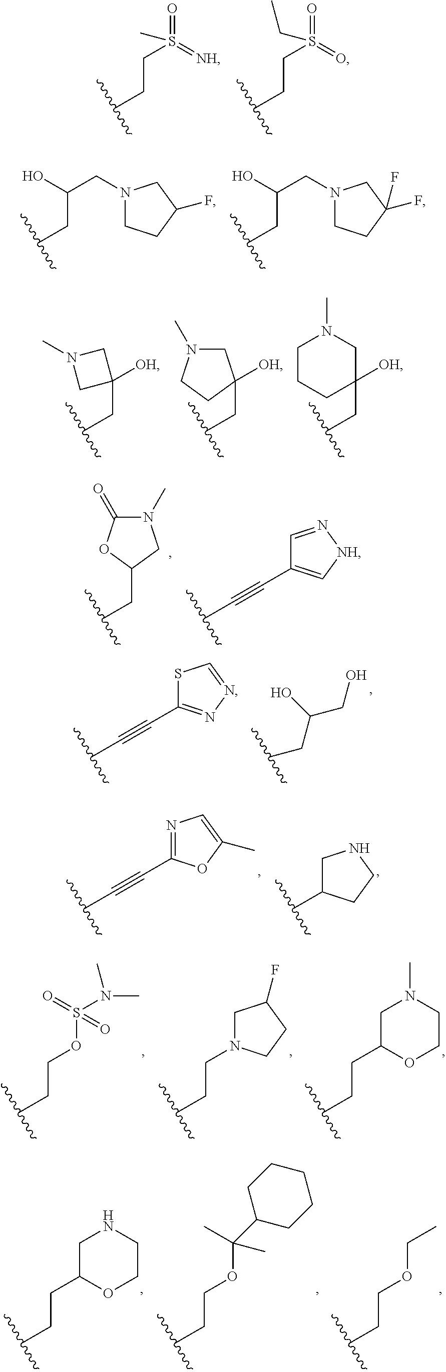

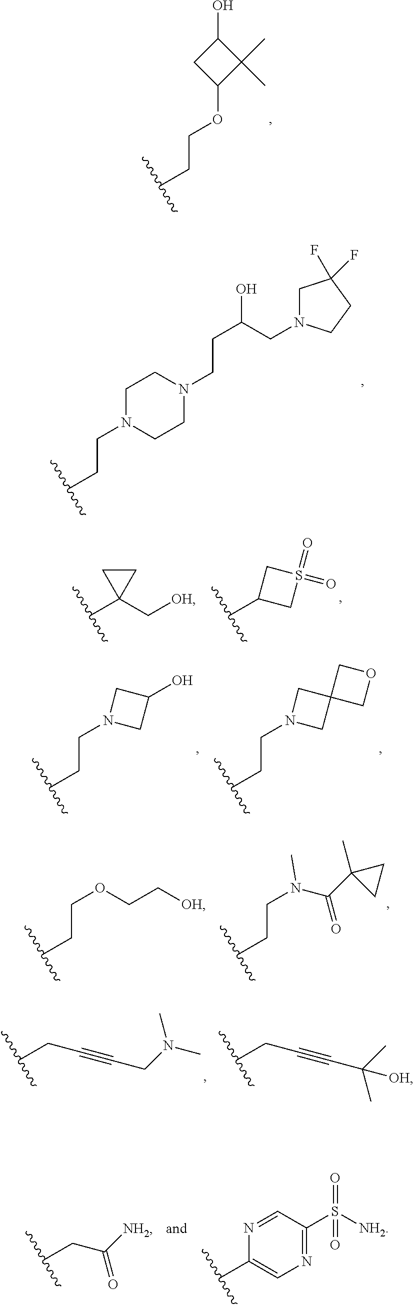

In one embodiment, W represents C(R^{W}). In an alternative embodiment, W represents N. In one embodiment, W represents C(H), C(CN), or N. In one embodiment, W represents C(Cl) or C(F).

In one embodiment, at least one of R¹ and R³ represents halogen or haloC₁₋₆ alkyl. In one embodiment, R¹ and R³ independently represent halogen or haloC₁₋₆ alkyl. In one embodiment, at least one of R¹ and R³ represents haloC₁₋₆ alkyl. In one embodiment, at least one of R¹ and R³ represents halogen. In one embodiment, R¹ and R³ each represent haloC₁₋₆ alkyl. In one embodiment, R¹ and R³ each independently represent halogen.

In one embodiment, R¹ represents hydrogen, deuterium, methyl, CD₃, haloC₁₋₆ alkyl (such as CF₃, CHF₂, or CH₂F), haloC₁₋₆ alkoxyl (such as OCF₃, OCHF₂, or OCH₂F), or halogen (such as fluorine or chlorine).

In one embodiment, R² represents hydrogen; deuterium C₁₋₆ alkyl (such as methyl or ethyl); C₂₋₆ alkenyl (such as ethenyl); C₁₋₆ alkoxy (such as methoxy); halogen (such as chlorine). In one embodiment, R² represents NR^{X}R^{Y} (such as N(Me)₂ or N(Me)(Et)). In one embodiment, R² represents: hydrogen; C₁₋₆ alkyl (such as methyl); C₁₋₆ alkoxy (such as methoxy). In one embodiment, R² represents —NR^{X}R^{Y} (such as —N(Me)₂ or —N(Me)(Et)). In one embodiment, R² represents C₁₋₆ alkyl (such as methyl). In one embodiment, R² represents: hydrogen; halogen (such as chlorine). In one embodiment, R² represents C₁₋₆ alkyl (such as methyl). In one embodiment, R² represents hydrogen.

In one embodiment, R³ represents hydrogen, deuterium, methyl, CD₃, haloC₁₋₆ alkyl (such as CF₃, CHF₂, or CH₂F), haloC₁₋₆ alkoxy (such as OCF₃, OCHF₂, or OCH₂F), or halogen (such as fluorine or chlorine). In one embodiment, R³ represents C₁₋₆ alkyl (such as methyl, ethyl or isopropyl); C₂₋₆ alkenyl (such as —C(═CH₂)(Me)); halogen (such as bromine); haloC₁₋₆ alkyl (such as trifluoromethyl or —C(H)(Me)-CF3).

In one embodiment, R³ represents haloC₁₋₆ alkoxy (such as difluoromethoxy). In one embodiment, R³ represents C₁₋₆ alkyl (such as methyl, ethyl or isopropyl).

In one embodiment, R³ represents haloC₁₋₆ alkyl (such as trifluoromethyl). In one embodiment, R³ represents haloC₁₋₆ alkyl (such as trifluoromethyl).

In one embodiment, R⁴ represents hydrogen, deuterium, C₁₋₆ alkyl (such as methyl, ethyl or isopropyl), CD₃, C₂ alkynyl, or nitrile. In one embodiment, R⁴ represents hydrogen.

In one embodiment, R⁵ represents CH₃ or CD₃. In one embodiment, R⁵ represents the following structure:

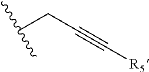

wherein R^{5′} is selected from the group consisting of hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof.

In one embodiment, R⁵ represents one of the following substituents:

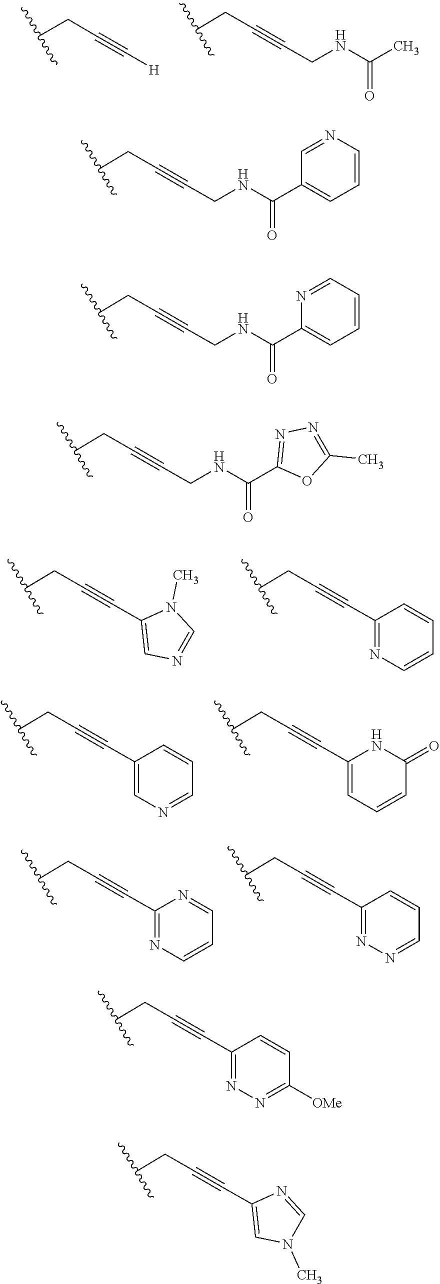

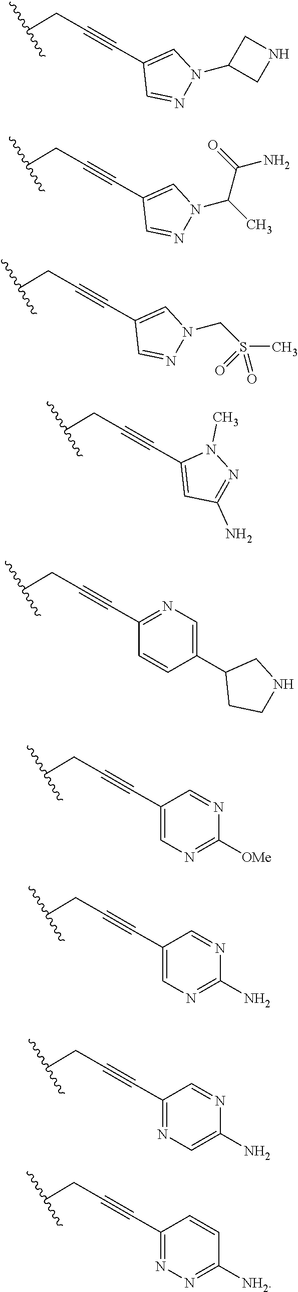

In one embodiment, R^{X} and R^{Y} represent C₁₋₆ alkyl (such as methyl or ethyl). In one embodiment, R^{X} and R^{Y} both represent C₁₋₆ alkyl (such as methyl or ethyl). In one embodiment, R^{X} and R^{Y} both represent methyl or one represents methyl and the other represents ethyl.

In one embodiment, W represents C(R⁴); R¹ represents C₁₋₆ alkyl (such as methyl), R² represents hydrogen, R³ represents haloC₁₋₆ alkyl (such as trifluoromethyl) and R⁴ represents nitrile.

In one embodiment, R¹ represents C₁₋₆ alkyl (such as methyl), R² represents hydrogen, R³ represents C₁₋₆ alkyl (such as methyl or isopropyl) and R⁴ represents nitrile.

In one embodiment, R¹ represents C₁₋₆ alkyl (such as methyl or ethyl), R² represents hydrogen, R³ represents haloC₁₋₆ alkyl (such as trifluoromethyl or —CH(Me)-CF3) and R⁴ represents hydrogen.

In one embodiment, R¹ represents C₁₋₆ alkyl (such as methyl), R² represents hydrogen, R³ represents C₁₋₆ alkyl (such as isopropyl) and R⁴ represents hydrogen.

In one embodiment, R¹ represents C₁₋₆ alkyl (such as methyl), R² represents halogen (such as chlorine), R³ represents C₁₋₆ alkyl (such as methyl) and R⁴ represents hydrogen.

In one embodiment, R¹ represents C₁₋₆ alkoxy (such as methoxy), R² represents hydrogen, R³ represents haloC₁₋₆ alkyl (such as trifluoromethyl) and R⁴ represents nitrile.

In one embodiment, R¹ represents —NR^{X}R^{Y} (such as —N(Me)2 or —N(Me)(Et)), R² represents hydrogen, R³ represents haloC₁₋₆ alkyl (such as trifluoromethyl), and R⁴ represents nitrile.

In one embodiment, R¹ represents hydrogen, R² represents hydrogen, R³ represents haloC₁₋₆ alkyl (such as trifluoromethyl) and R⁴ represents nitrile.

In one embodiment, R¹ represents hydrogen, R² represents C₁₋₆ alkyl (such as methyl), R³ represents C₁₋₆ alkyl (such as ethyl) and R⁴ represents hydrogen.

In one embodiment, R¹ represents halogen (such as chlorine), R² represents hydrogen, R³ represents haloC₁₋₆ alkyl (such as trifluoromethyl) and R⁴ represents hydrogen.

In one embodiment, R¹ represents halogen (such as chlorine), R² represents hydrogen, R³ represents haloC₁₋₆ alkoxy (such as difluoromethoxy) and R⁴ represents hydrogen.

In one embodiment, R¹ represents C₂₋₆ alkenyl (such as ethenyl), R² represents hydrogen, R³ represents haloC₁₋₆ alkyl (such as trifluoromethyl) and R⁴ represents hydrogen.

In one embodiment, R¹ represents C₁₋₆ alkyl (such as methyl), R² represents hydrogen, R³ represents haloC₁₋₆ alkoxy (such as difluoromethoxy) and R⁴ represents hydrogen.

In one embodiment, R¹ represents C₁₋₆ alkyl (such as methyl), R² represents hydrogen, R³ represents C₂₋₆ alkenyl (such as —C(Me)(═CH₂)) and R⁴ represents hydrogen.

In one embodiment, R¹ represents C₁₋₆ alkyl (such as methyl), R² represents hydrogen, R³ represents halogen (such as bromine) and R⁴ represents hydrogen.

In one embodiment, R¹ represents C₁₋₆ alkyl (such as methyl), R² represents hydrogen, R³ represents haloC₁₋₆ alkyl (such as trifluoromethyl) and R⁴ represents nitrile;

In one embodiment, R¹ represents C₁₋₆ alkyl (such as methyl), R² represents hydrogen, R³ represents C₁₋₆ alkyl (such as methyl or isopropyl) and R⁴ represents nitrile.

In one embodiment, R¹ represents C₁₋₆ alkyl (such as methyl), R² represents hydrogen, R³ represents haloC₁₋₆ alkyl (such as trifluoromethyl) and R⁴ represents hydrogen.

In one embodiment, R¹ represents C₁₋₆ alkyl (such as methyl), R² represents hydrogen, R³ represents C₁₋₆ alkyl (such as isopropyl) and R⁴ represents hydrogen.

In one embodiment, R¹ represents C₁₋₆ alkyl (such as methyl), R² represents halogen (such as chlorine), R³ represents C₁₋₆ alkyl (such as methyl) and R⁴ represents hydrogen.

In one embodiment, R¹ represents C₁₋₆ alkoxy (such as methoxy), R² represents hydrogen, R³ represents haloC₁₋₆ alkyl (such as trifluoromethyl) and R⁴ represents nitrile.

In one embodiment, R¹ represents —NR^{X}R^{Y} (such as —N(Me)₂ or —N(Me)(Et)), R² represents hydrogen, R³ represents haloC₁₋₆ alkyl (such as trifluoromethyl) and R⁴ represents nitrile.

In one embodiment, R¹ represents hydrogen, R² represents hydrogen, R³ represents haloC₁₋₆ alkyl (such as trifluoromethyl) and R⁴ represents nitrile.

In one embodiment, R¹ represents hydrogen, R² represents C₁₋₆ alkyl (such as methyl), R³ represents C₁₋₆ alkyl (such as ethyl) and R⁴ represents hydrogen.

In one embodiment, R¹ represents C₁₋₆ alkyl (such as methyl), R² represents hydrogen, R³ represents haloC₁₋₆ alkyl (such as trifluoromethyl) and R⁴ represents hydrogen.

In one embodiment, W represents N, R¹ represents C₁₋₆ alkyl (such as methyl), R² represents hydrogen and R³ represents haloC₁₋₆ alkyl (such as trifluoromethyl).

In one embodiment, X represents C(R¹⁵)(R¹⁶) or O. In one embodiment, X represents C(R¹⁵)(R¹⁶). In one embodiment, X represents O. In one embodiment, X represents —N(R¹⁷)—.

In one embodiment, Z represents CH₂ or C═O. In one embodiment, Z represents CH₂. In one embodiment, Z represents C═O. In one embodiment, Z represents C(R^{Z})(H).

In one embodiment, Z represents C(CH₃)(H).

In one embodiment, R¹⁵ and R¹⁶ independently represent hydrogen; —OR^{15a} (such as hydroxy); halogen (such as fluorine); C₁₋₆ alkanol (such as CH₂OH); C₁₋₆ alkoxy (such as methoxy); —NR^{V}R^{W} (such as —NH₂, —NMe₂, —N(H)(Me), —N(H)(COMe), —N(H)((CH₂)₂OH), —N(H)((CH₂)₂SO₂Me), N(Me)((CH₂)₂SO₂Me), —N(H)(pyrrolidinyl), —N(Me)(pyrrolidinyl), —N(H)(azetidinyl), —N(H)(oxetanyl), —N(Me)(oxetanyl), —N(H)(cyclopentyl), —N(Me)(cyclopentyl), —N(H)(tetrahydropyranyl), —N(Me)(tetrahydropyranyl) or —N(H)((CH₂)₂NH₂), wherein said pyrrolidinyl, tetrahydropyranyl or cyclopentyl rings may be optionally substituted by one or more oxo, hydroxy, —COC₁₋₆ alkyl (such as —COMe) or —COOC₁₋₆ alkyl (such as —COOtBu) groups; —CH₂—NR^{V}R^{W} (such as —CH₂—N(Me)₂); L-aryl (such as —CH₂—O—CH₂-phenyl).

In one embodiment, heterocyclyl (such as azetidinyl, pyrrolidinyl, morpholinyl or piperazinyl) optionally substituted by one or more hydroxy or C₁₋₆ alkanol (such as CH₂OH) groups.

In one embodiment, R¹⁵ and R¹⁶ independently represent hydrogen; hydroxy; halogen (such as fluorine); C₁₋₆ alkoxy (such as methoxy); —NR^{V}R^{W} (such as —NH₂, —NMe₂, —N(H)(Me), —N(H)(COMe), —N(H)((CH₂)₂OH), —N(H)((CH₂)₂SO₂Me), N(Me)((CH₂)₂SO₂Me), —N(H)(pyrrolidinyl), —N(Me)pyrrolidinyl), —N(H)(azetidinyl), —N(H)((CH₂)₂NH₂), wherein said pyrrolidinyl ring may be optionally substituted by an oxo or —COC₁₋₆ alkyl (such as —COMe) group.

In one embodiment, heterocyclyl (such as morpholinyl or piperazinyl).

In one embodiment, R¹⁵ represents hydroxy.

In one embodiment, R^{A}, R^{A′}, R^{B}, and R^{B′} each represents hydrogen or —OR¹⁶ (such as hydroxy).

In one embodiment, R^{A}, R^{A′}, R^{B}, and R^{B′} each represents hydrogen or hydroxy.

In one embodiment, R^{B} represents —OR^{15b} (such as hydroxy).

In one embodiment, X represents —C(H)(R¹⁶)— and R^{Z}, R^{Z′}, R¹⁶, R^{A}, R^{A′}, R^{B}, and R^{B′}, each represent hydrogen. In one embodiment, X represents —C(H)(R¹⁶)— and R¹⁶ represents —OR^{15a} (such as hydroxy). In one embodiment, X represents —C(H)(R¹⁶)—, R^{Z}, R^{Z′}, R¹⁵, and R¹⁶ represent hydrogen, and R^{B} represents —OR^{15b} (such as hydroxy). In one embodiment, R¹⁶ represents halogen (such as fluorine). In one embodiment, R¹⁶ represents C₁₋₆ alkoxy (such as methoxy). In one embodiment, R¹⁶ represents —NR^{V}R^{W} (such as —NH₂, —NMe₂, —N(H)(Me), —N(H)((CH₂OH), —N(H)((CH₂)₂SO₂Me), N(Me)((CH₂)₂SO₂Me), —N(H)(pyrrolidinyl), —N(Me)(pyrrolidinyl), —N(H)(azetidinyl), —N(H)(oxetanyl), —N(Me)(oxetanyl), —N(H)(cyclopentyl), —N(Me)(cyclopentyl), —N(H)(tetrahydropyranyl), —N(Me)(tetrahydropyranyl) or —N(H)((CH₂)₂NH₂), wherein said pyrrolidinyl, tetrahydropyranyl or cyclopentyl rings may be optionally substituted by one or more oxo, hydroxy, —COC₁₋₆ alkyl (such as —COMe) or —COOC₁₋₆ alkyl (such as —COOtBu) groups. In one embodiment, R¹⁶ represents heterocyclyl (such as azetidinyl, pyrrolidinyl, morpholinyl or piperazinyl optionally substituted by one or more hydroxy or C₁₋₆ alkanol (i.e. CH₂OH) groups). In one embodiment, R¹⁶ and R^{B} each represent OR^{15b} (such as hydroxy).

In one embodiment, Z represents C═O, X represents CR¹⁵R¹⁶, and R¹⁵, R¹⁶, R^{A}, R^{A′}, R^{B}, and R^{B′} each represent hydrogen. In one embodiment, Z represents C═O, X represents CHR¹⁶, R¹⁶ represents —NR^{V}R^{W} (such as —NH₂ or —N(H)(COMe)) and R^{A}, R^{A′}, R^{B}, and R^{B′} each represent hydrogen. In one embodiment, Z represents C═O, X represents CR¹⁵R¹⁶, R¹⁵, R¹⁶, R^{A}, R^{A′}, R^{B}, and R^{B′} each represent hydrogen, and R¹⁶ represents heterocyclyl (such as morpholinyl). In one embodiment, Z represents C═O, X represents CR¹⁵R¹⁶, and R¹⁵, R^{A}, R^{A′}, and R^{B′} each represent hydrogen, R¹⁶ represents —OR^{15a} (such as hydroxy), and R^{B} represents OR^{15b} (such as hydroxy). In one embodiment, Z represents C═O, X represents CR¹⁵R¹⁶, and R¹⁵, R^{A}, R^{A′}, R^{B}, and R^{B′} each represent hydrogen, and R¹⁶ represents —OR^{15a} (such as hydroxy). In one embodiment, Z represents C═O, X represents CR¹⁵R¹⁶, and R¹⁵, R^{A}, R^{A′}, R^{B}, and R^{B′} each represent hydrogen, and R¹⁶ represents -L-aryl (such as —CH₂—O—CH₂-phenyl). In one embodiment, In one embodiment, Z represents C═O, X represents CR¹⁵R¹⁶, and R¹⁵, R^{A}, R^{A′}, R^{B}, and R^{B′} each represent hydrogen, and R¹⁶ represents C₁₋₆ alkanol (such as CH₂OH). In one embodiment, represents C═O, X represents CR¹⁵R¹⁶, and R¹⁵, R¹⁶, R^{A}, R^{A′}, and R^{B′} each represent hydrogen, and R^{B} represents —OR^{15b} (such as hydroxy). In one embodiment, Z represents C═O, X represents CR¹⁵R¹⁶, and R¹⁵, R^{A}, R^{A′}, R^{B}, and R^{B′} each represent hydrogen, and R¹⁶ represents CH₂—NR^{V}R^{W} (such as —CH₂—N(Me)₂). In one embodiment,

In one embodiment, Z represents C═O, X represents CR¹⁵R¹⁶; R¹⁵, R^{A}, R^{A′}, and R^{B′} each represent hydrogen; R¹⁶ represents —OR^{15a}; R^{B} represents —OR^{15b}; and R^{15a} and R^{15b} join together to form a 5 to 7 (such as 5) membered saturated ring system (such as dioxolanyl) which may be optionally substituted by one or more C₁₋₆ alkyl groups (such as two methyl groups). In one embodiment, Z represents C═O, X represents CR¹⁵R¹⁶; R¹⁵, R^{A}, R^{A′}, and R^{B′} each represent hydrogen; R¹⁶ represents —OR^{15a}; R^{B} represents —OR^{15b}; and R^{15a} and R^{15b} join together to form a 5 to 7 (such as 5) membered saturated ring system (such as dioxolanyl) which may be optionally substituted by one or more C₁₋₆ alkyl groups (such as two methyl groups).

In one embodiment, R¹⁵, R¹⁶, R^{A}, R^{A′}, R^{B}, and R^{B′} each represent hydrogen and R^{Z} represents methyl. In one embodiment, In one embodiment, X represents —C(H)(R¹⁶)— and R¹⁶, R^{A}, R^{A′}, R^{B}, and R^{B′} each represent hydrogen. In one embodiment, X represents —C(H)(R¹⁶)—; R¹⁶, R^{Z}, R^{Z′}, R^{A}, R^{A′}, R^{B}, and R^{B′} each represent hydrogen. In one embodiment, R^{Z}, R^{Z′}, R^{A}, R^{A′}, R^{B}, and R^{B′} each represent hydrogen and R¹⁶ represents

—OR^{15a} (such as hydroxy). In one embodiment, R¹⁶, R^{Z}, R^{Z′}, R^{A}, R^{A′}, and R^{B′} each represent hydrogen; and R^{B} represents —OR^{15b} (such as hydroxy). In one embodiment, R^{Z}, R^{Z′}, R^{A}, R^{A′}, R^{B}, and R^{B′} represent hydrogen and R¹⁶ represents halogen (such as fluorine). In one embodiment, R^{Z}, R^{Z′}, R^{A}, R^{A′}, R^{B}, and R^{B′} represent hydrogen and R¹⁶ represents C₁₋₆ alkoxy (such as methoxy). In one embodiment, R^{Z}, R^{Z′}, R^{A}, R^{A′}, R^{B}, and R^{B′} represent hydrogen and R¹⁶ represents represents —NR^{V}R^{W} (such as —NH₂, —NMe₂, —N(H)(Me), —N(H)((CH₂)₂OH), —N(H)((CH₂)₂SO₂Me), N(Me)(CH₂)₂SO₂Me) —N(H)(pyrrolidinyl), —N(Me)(pyrrolidinyl), —N(H)(azetidinyl), —N(H)(oxetanyl), —N(Me)(oxetanyl), —N(H)(cyclopentyl), —N(Me)(cyclopentyl), —N(H)(tetrahydropyranyl), —N(Me)tetrahydropyranyl) or —N(H)((CH₂)₂NH₂), wherein said pyrrolidinyl, tetrahydropyranyl or cyclopentyl rings may be optionally substituted by one or more oxo, hydroxy, —COC₁₋₆ alkyl (such as —COMe) or —COOC₁₋₆ alkyl (such as —COOtBu) groups. In one embodiment, R^{Z}, R^{Z′}, R^{A}, R^{A′}, R^{B}, and R^{B′} represent hydrogen and R¹⁶ represents represents heterocyclyl (such as azetidinyl, pyrrolidinyl, morpholinyl or piperazinyl optionally substituted by one or more hydroxy or C₁₋₆ alkanol (i.e. CH₂OH) groups). In one embodiment, In one embodiment, R^{Z}, R^{Z′}, R^{A}, R^{A′}, and R^{B′} represent hydrogen; R¹⁶ represents —OR^{15a} (such as hydroxy); and R^{B} represents OR^{15b} (such as hydroxy).

In one embodiment, Z represents C═O; X represents —C(H)(R¹⁶)—; and R¹⁶, R^{A}, R^{A′}, R^{B}, and R^{B′} each represent hydrogen. In one embodiment, Z represents C═O; X represents —C(H)(R¹⁶)—; R^{A}, R^{A′}, R^{B}, and R^{B′} each represent hydrogen; and R¹⁶ represents —NR^{V}R^{W} (such as -IMH₂ or —N(H)(COMe)). In one embodiment, Z represents C═O; X represents —C(H)(R¹⁶)—; R^{A}, R^{A′}, R^{B}, and R^{B′} each represent hydrogen; and R¹⁶ represents heterocyclyl (such as morpholinyl). In one embodiment, Z represents C═O; X represents —C(H)(R¹⁶)—; R^{A}, R^{A′}, R^{B}, and R^{B′} each represent hydrogen; R¹⁶ represents —OR^{15a} (such as hydroxy); and R^{B} represents —OR^{15b} (such as hydroxy). In one embodiment, Z represents C═O; X represents —C(H)(R¹⁶)—; R^{A}, R^{A′}, R^{B}, and R^{B′} each represent hydrogen; and R¹⁶ represents —OR^{15a} (such as hydroxy). In one embodiment, Z represents C═O; X represents —C(H)(R¹⁶)—; R^{A}, R^{A′}, R^{B}, and R^{B′} each represent hydrogen; and R¹⁶ represents -L-aryl (such as —CH₂—O—CH₂-phenyl). In one embodiment, Z represents C═O; X represents —C(H)(R¹⁶)—; R^{A}, R^{A′}, R^{B}, and R^{B′} each represent hydrogen; and R¹⁶ represents C₁₋₆ alkanol (such as CH₂OH). In one embodiment, Z represents C═O; X represents —C(H)(R¹⁶)—; R^{A}, R^{A′}, R^{B}, and R^{B′} each represent hydrogen; and R^{B} represents —OR^{15b} (such as hydroxy). In one embodiment, Z represents C═O; X represents —C(H)(R¹⁶)—; R^{A}, R^{A′}, R^{B}, and R^{B′} each represent hydrogen; and R¹⁶ represents —CH₂—NR^{V}R^{W} (such as —CH₂—N(Me)₂). In one embodiment, Z represents C═O; X represents —C(H)(R¹⁶)—; R^{A}, R^{A′}, and R^{B′} each represent hydrogen; R¹⁶ represents —OR^{15a} and R^{B} represents —OR^{15b} (such as hydroxy) wherein R^{15a} and R^{15b} join together to form a 5 to 7 (such as 5) membered saturated ring system (such as dioxolanyl) which may be optionally substituted by one or more C₁₋₆ alkyl groups (such as two methyl groups).

In one embodiment, X represents —C(H)(R¹⁶)— and R^{A}, R^{A′}, R^{B}, and R^{B′} each represent hydrogen. In one embodiment, R¹⁶ represents hydroxyl. In one embodiment, R^{B} represents hydroxy. In one embodiment, R¹⁶ represents halogen (such as fluorine). In one embodiment, R¹⁶ represents C₁₋₆ alkoxy (such as methoxy). In one embodiment, R¹⁶ represents —NR^{V}R^{W} (such as —NH₂, —NMe₂, —N(H)(Me), —N(H)((CH₂)₂OH), —N(H)((CH₂)₂SO₂Me), N(Me)((CH₂)₂SO₂Me), —N(H)(pyrrolidinyl), —N(Me)(pyrrolidinyl), —N(H)(azetidinyl), —N(H)((CH₂)₂NH₂), wherein said pyrrolidinyl ring may be optionally substituted by an oxo or —COC₁₋₆ alkyl (such as —COMe) group. In one embodiment, R¹⁶ represents heterocyclyl (such as morpholinyl or piperazinyl). In one embodiment, R¹⁶ and R^{B} both represent hydroxy.

In one embodiment, Z represents C═O; X represents —C(H)(R¹⁶)—; and R¹⁶, R^{A}, R^{A′}, R^{B}, and R^{B′} each represent hydrogen. In one embodiment, R¹⁶ represents —NR^{V}R^{W} (such as —NH₂ or —N(H)(COMe)). In one embodiment, R¹⁶ represents heterocyclyl (such as morpholinyl). In one embodiment, R¹⁶ and R^{B} both represent hydroxy. In one embodiment, Z represents C═O; X represents —C(H)(R¹⁶)—; and R¹⁶ and R^{B} both represent hydroxy.

In one embodiment, Z represents C═O; X represents O; and R^{A}, R^{A′}, R^{B}, and R^{B′} each represent hydrogen.

In one embodiment, Z represents C═O; X represents —N(R¹⁷)—, and R¹⁷ represents hydrogen. In one embodiment, Z represents C═O; X represents —N(R¹⁷)—, and R¹⁷ represents C₁₋₆ alkanol (such as —CH₂—CH(OH)Me, —(CH₂)₂—OH, —CH₂—CHOH—CH₂OH or —(CH₂)₂—CHOH—CH₂OH); -L-SO₂—C₁₋₆ alkyl (such as —SO₂-Me or —(CH₂)₂—SO₂-Me); -L-SO₂—NR^{V}R^{W} (such as —(CH₂)₂—SO₂—N(Me)₂); -L-NR^{V}R^{W} (such as —(CH₂)₂—N(Me)₂, —(CH₂)₃—N(Me)₂ or —CH₂—CHOH—CH₂—NMe₂); -L-CO—NR^{V}R^{W} (such as —CH₂—CONH₂, —CH₂—CON(Me)₂, —(CH₂)₂—CON(Me)₂ or —(CH₂)₂—CON(H)(Me)); -L-NH—SO₂—C₁₋₆ alkyl (such as —(CH₂)₂—NH—SO₂-Me); -L-S(═NH)(═O)(C₁₋₆ alkyl) (such as —(CH₂)₂—S(=NH)(═O)(Me)); -L-O—SO₂—NR^{V}R^{W} (such as —(CH₂)₂—O—SO₂—NH₂); -L-N═S(═O)(C₁₋₆ alkyl)₂ (such as —(CH₂)₂—N═S(═O)(Me)₂).

In one embodiment, -L-heterocyclyl (such as —CH₂-oxetanyl, —CH₂-azetidinyl, —(CH₂)₂-azetidinyl, —CH₂-oxazolidinyl, —(CH₂)₂-piperidinyl, —(CH₂)₂-piperazinyl, —(CH₂)₃-piperazinyl, —CH₂-morpholinyl, —(CH₂)₂-morpholinyl, —CH₂—CHOH—CH₂-morpholinyl, —(CH₂)₂-thiomorpholinyl, —CH₂-pyrrolidinyl, —(CH₂)₂-pyrrolidinyl or —CH₂—CHOH—CH₂-pyrrolidinyl), wherein said heterocyclyl ring may be optionally substituted by one or more oxo, hydroxy, halogen (such as fluorine), nitrile, C₁₋₆ alkyl (such as methyl), —COC₁₋₆ alkyl (such as —COMe), —NR^{V}—COC₁₋₆ alkyl (such as —NMe-COMe) or C₁₋₆ alkanol (such as —CH₂OH or —(CH₂)₂—OH) groups.

In one embodiment, Z represents C═O; X represents —N(R¹⁷)—, R^{B} represents hydrogen, and R¹⁷ represents: C₁₋₆ alkanol (such as —CH₂—CH(OH)Me, —(CH₂)₂—OH, —CH₂—CHOH—CH₂OH or —(CH₂)₂—CHOH—CH₂OH); -L-SO₂—C₁₋₆ alkyl (such as —SO₂-Me or —(CH₂)₂—SO₂-Me); -L-SO₂—NR^{V}R^{W} (such as —(CH₂)₂—SO₂—N(Me)₂); -L-NR^{V}R^{W} (such as —(CH₂)₂—N(Me)₂, —(CH₂)₃—N(Me)₂ or —CH₂—CHOH—CH₂—NMe₂); -L-CO—NR^{V}R^{W} (such as —CH₂—CONH₂, —CH₂—CON(Me)₂, —(CH₂)₂—CON(Me)₂ or —(CH₂)—CON(H)(Me)); -L-NH—SO₂—C₁₋₆ alkyl (such as —(CH₂)₂—NH—SO₂-Me); -L-S(═NH)(═O)(C₁₋₆ alkyl) (such as —(CH₂)₂—S(═NH)(═O)(Me)); -L-O—SO₂—NR^{V}R^{W} (such as —(CH₂)—O—SO₂—NH₂); -L-N═S(═O)(C₁₋₆ alkyl)₂ (such as —(CH₂)₂—N═S(═O)(Me)₂).

In one embodiment, R¹⁷ represents -L-heterocyclyl (such as —CH₂-oxetanyl, —CH₂-azetidinyl, —(CH₂)₂-azetidinyl, —CH₂-oxazolidinyl, —(CH₂)₂-piperidinyl, —(CH₂)-piperazinyl, —(CH₂)₃-piperazinyl, —CH₂-morpholinyl, —(CH₂)₂-morpholinyl, —CH₂—CHOH—CH₂-morpholinyl, —(CH₂)₂-thiomorpholinyl, —CH₂-pyrrolidinyl, —(CH₂)₂-pyrrolidinyl or —CH₂—CHOH—CH₂-pyrrolidinyl), wherein said heterocyclyl ring may be optionally substituted by one or more oxo, hydroxy, halogen (such as fluorine), nitrile, C₁₋₆ alkyl (such as methyl), —COC₁₋₆ alkyl (such as —COMe), —NR^{V}—COC₁₋₆ alkyl (such as —NMe-COMe) or C₁₋₆ alkanol (such as —CH₂OH or —(CH₂)₂—OH) groups.

In one embodiment, Z represents C═O; X represents —N(R¹⁷)—; and R^{B} and R¹⁷ represent hydrogen. In one embodiment, R¹⁷ represents C₁₋₆ alkanol (such as —CH₂—CH(OH)Me). In one embodiment, R¹⁷ represents —SO₂—C₁₋₆ alkyl (such as —SO₂-Me).

In one embodiment, R^{V} and R^{W} represent hydrogen, C₁₋₆ alkyl (such as methyl), —COC₁₋₆ alkyl (such as —COMe), C₃₋₈ cycloalkyl (such as cyclopentyl) or heterocyclyl (such as oxetanyl, azetidinyl, tetrahydropyranyl or pyrrolidinyl), wherein said alkyl groups may be optionally substituted with or more hydroxy (such as (Chb\hat{ }OH), amino (such as (Chb\hat{ }Nhb) or sulfone (such as (CH₂)₂SO₂Me) groups and said heterocyclyl ring may be optionally substituted by one or more oxo or —COC₁₋₆ alkyl (such as —COMe) groups. In one embodiment, R^{V} and R^{W} represent hydrogen, C₁₋₆ alkyl (such as methyl), —COC₁₋₆ alkyl (such as —COMe) or heterocyclyl (such as azetidinyl or pyrrolidinyl), wherein said alkyl groups may be optionally substituted with or more hydroxy (such as (Chb\hat{ }OH), amino (such as (CH₂)₂NH₂) or sulfone (such as (CH₂)₂SO₂Me) groups and said heterocyclyl ring may be optionally substituted by one or more oxo or —COC₁₋₆ alkyl (such as —COMe) groups. In one embodiment, R^{V} and R^{W} both represent hydrogen or C₁₋₆ alkyl (such as methyl) or one represents hydrogen and the other represents C₁₋₆ alkyl (such as methyl) or one represents hydrogen or C₁₋₆ alkyl (such as methyl) and the other represents —COC₁₋₆ alkyl (such as —COMe), C₃₋₈ cycloalkyl (such as cyclopentyl) or heterocyclyl (such as azetidinyl, tetrahydropyranyl or pyrrolidinyl), wherein said alkyl groups may be optionally substituted with or more hydroxy (such as (Chb\hat{ }OH), amino (such as (Chb\hat{ }Nhb) or sulfone (such as (CH₂)₂SO₂Me) groups and said heterocyclyl ring may be optionally substituted by one or more oxo or —COC₁₋₆ alkyl (such as —COMe) groups. In one embodiment, R^{V} and R^{W} both represent hydrogen or C₁₋₆ alkyl (such as methyl) or one represents hydrogen and the other represents C₁₋₆ alkyl (such as methyl) or one represents hydrogen or C₁₋₆ alkyl (such as methyl) and the other represents —COC₁₋₆ alkyl (such as —COMe) or heterocyclyl (such as azetidinyl or pyrrolidinyl), wherein said alkyl groups may be optionally substituted with or more hydroxy (such as (Chb\hat{ }OH), amino (such as (Chb\hat{ }Nhb) or sulfone (such as (CH₂)₂SO₂Me) groups and said heterocyclyl ring may be optionally substituted by one or more oxo or —COC₁₋₆ alkyl (such as —COMe) groups.

In one embodiment, R⁵ represents C₁₋₆ alkyl (such as CH₃, CD₃, ethyl or isopropyl) or C₃₋₈ cycloalkyl (such as cyclopropyl). In one embodiment, R⁵ represents C₁₋₆ alkyl (such as methyl, ethyl or isopropyl). In one embodiment, R⁵ represents C₁₋₆ alkyl (such as CH₃, CD₃ or ethyl). In one embodiment, R⁵ represents C₁₋₆ alkyl (such as methyl or ethyl). In a yet further embodiment, R⁵ represents C₁₋₆ alkyl (such as CHs or CDs).

In one embodiment, Y represents —C(R⁶)═. In an alternative embodiment, Y represents N. In one embodiment, R⁶ represents: hydrogen; halogen (such as fluorine or chlorine).

In one embodiment, R⁶ represents C₁₋₆ alkoxy (such as methoxy). In one embodiment, R⁶ represents: hydrogen; halogen (such as fluorine). In one embodiment, R⁶ represents C₁₋₆ alkoxy (such as methoxy). In one embodiment, R⁶ represents: hydrogen. In one embodiment, R⁶ represents halogen (such as fluorine). In one embodiment, R⁶ represents hydrogen.

In one embodiment, R⁷ represents: hydrogen; halogen (such as fluorine, bromine or chlorine); C₁₋₆ alkyl (such as methyl or ethyl); C₂₋₆ alkenyl (such as ethenyl); hydroxy; C₁₋₆ alkoxy (such as methoxy); —NR^{X}R^{Y} (such as —NH₂, —NHMe or —NMe₂). In one embodiment, R⁷ represents: hydrogen; halogen (such as fluorine, bromine or chlorine); C₁₋₆ alkyl (such as methyl, ethyl, CD₃, or C₂D₅); C₂₋₆ alkenyl (such as ethenyl); hydroxy; C₁₋₆ alkoxy (such as methoxy). In one embodiment, R⁷ represents: hydrogen; halogen (such as fluorine or chlorine).

In one embodiment, R^{X} and R^{Y} represent C₁₋₆ alkyl (such as methyl). In one embodiment, R⁷ represents halogen (such as chlorine). In one embodiment, R^{X} and R^{Y} independently represent hydrogen or methyl. In one embodiment, both of R^{X} and R^{Y} represent hydrogen or both of R^{X} and R^{Y} represent methyl or one of R^{X} and R^{Y} represents hydrogen and the other represents methyl.

In one embodiment, R⁸, R⁹, and R¹⁰ each represent halogen. In one embodiment, R⁸¹ represents fluorine. In one embodiment, R⁹ represents chlorine. In one embodiment, R¹⁰ represents fluorine.

In one embodiment, R⁸ represents: hydrogen; halogen (such as fluorine, bromine or chlorine); C₁₋₆ alkyl (such as methyl or ethyl); C₂₋₆ alkenyl (such as ethenyl); C₃₋₈ cycloalkyl (such as cyclopropyl); haloC₁₋₆ alkyl (such as trifluoromethyl). In one embodiment, R⁸ represents: hydrogen; halogen (such as fluorine, bromine or chlorine); C₁₋₆ alkyl (such as methyl or ethyl); C₂₋₆ alkenyl (such as ethenyl); haloC₁₋₆ alkyl (such as trifluoromethyl). In one embodiment, R⁸ and R⁷ join to form a 5 to 7 membered saturated or unsaturated ring optionally containing one or more heteroatoms selected from O, N or S (such as a pyrrolinyl or tetrahydropyranyl ring). In one embodiment, R⁸ represents: hydrogen; halogen (such as fluorine or chlorine).

In one embodiment, R⁸ represents haloC₁₋₆ alkyl (such as trifluoromethyl). In one embodiment, R⁸ represents halogen (such as fluorine).

In one embodiment, R⁷ and R⁸ join to form a 5 to 7 membered saturated or unsaturated ring optionally containing one or more heteroatoms selected from O, N or S (such as a benzyl, pyridinyl, purinyl, pyrimidinyl, diazinyl, pyrrolyl, pyrrolinyl, tetrahydropyranyl, pyrazolyl, morpholinyl, pyridyl, furanyl or thiophenyl ring optionally substituted by one or more methyl or fluorine groups). In one embodiment, R⁷ and R⁸ join to form a 5 to 7 membered saturated or unsaturated ring optionally containing one or more heteroatoms selected from O, N or S (such as a pyrrolinyl or tetrahydropyranyl ring). In one embodiment, R⁸ and R⁷ join to form a 5 to 7 membered saturated or unsaturated ring optionally containing one or more heteroatoms selected from O, N or S (such as a benzyl, pyridinyl, purinyl, pyrimidinyl, diazinyl, pyrrolyl, pyrrolinyl, tetrahydropyranyl, pyrazolyl, morpholinyl, pyridyl, furanyl or thiophenyl ring optionally substituted by one or more methyl or fluorine groups).

In one embodiment, R⁹ represents: hydrogen; halogen (such as fluorine or chlorine); C₁₋₆ alkyl (such as methyl); haloC₁₋₆ alkyl (such as fluoromethyl, difluoromethyl or trifluoromethyl). In one embodiment, R⁹ represents C₁₋₆ alkoxy (such as methoxy). In one embodiment, R⁹ represents hydrogen.

In one embodiment, R¹⁰ represents: hydrogen.

In one embodiment, R¹⁰ represents halogen (such as fluorine). In one embodiment, R¹⁰ represents hydrogen.

In one embodiment, Y represents —C(R⁶)═; and each of R⁶, R⁷, R⁸, R⁹ and R¹⁰ represent hydrogen.

In one embodiment, each of R⁶, R⁷, R⁸ and R¹⁰ represent hydrogen and R⁹ represents C₁₋₆ alkyl (such as methyl).

In one embodiment, each of R⁶, R⁷ and R¹⁰ represent hydrogen and R⁸ and R⁹ both represents halogen (such as fluorine or chlorine).

In one embodiment, each of R⁶, R⁷ and R¹⁰ represent hydrogen, R represents halogen (such as fluorine) and R⁹ represents C₁₋₆ alkyl (such as methyl).

In one embodiment, each of R⁶, R⁷ and R¹⁰ represent hydrogen, R⁸ represents halogen (such as fluorine) and R⁹ represents haloC₁₋₆ alkyl (such as fluoromethyl or trifluoromethyl).

In one embodiment, each of R⁶, R⁷, R⁸ and R¹⁰ represent hydrogen and R⁹ represents haloC₁₋₆ alkyl (such as fluoromethyl or difluoromethyl).

In one embodiment, each of R⁶, R⁷, R⁸ and R¹⁰ represent hydrogen and R⁹ represents halogen (such as chlorine).

In one embodiment, each of R⁶, R⁷ and R¹⁰ represent hydrogen, R⁸ represents halogen (such as fluorine) and R¹ represents C₁₋₆ alkoxy (such as methoxy).

In one embodiment, each of R⁶, R⁸ and R¹⁰ represent hydrogen and R⁸ and R⁹ both represent halogen (such as fluorine or chlorine).

In one embodiment, each of R⁶, R⁷ and R¹⁰ represent hydrogen, R⁸ represents haloC₁₋₆ alkyl (such as trifluoromethyl) and R⁹ represents halogen (such as fluorine).

In one embodiment, each of R⁶, R⁸, R⁹ and R¹⁰ represent hydrogen and R⁷ represents C₁₋₆ alkyl (such as methyl, ethyl, CD₃, or C₂D₅); each of R⁶, R⁸, R⁹ and R¹⁰ represent hydrogen and R⁷ represents halogen (such as chlorine); each of R⁶, R⁹ and R¹⁰ represent hydrogen and R⁷ and R⁸ both represent halogen (such as fluorine, bromine or chlorine); each of R⁶, R⁹, and R¹⁰ represent hydrogen, R⁷ represents C₁₋₆ alkyl (such as methyl, ethyl, CD₃, or C₂D₅), and R⁸ represents halogen (such as fluorine); each of R⁶, R⁷ and R¹⁰ represent hydrogen, R⁸ represents C₁₋₆ alkyl (such as methyl) and R⁹ represents halogen (such as chlorine); each of R⁶, R⁹ and R¹⁰ represent hydrogen, R⁷ represents C₁₋₆ alkyl (such as methyl or ethyl) and R⁸ represents halogen (such as chlorine or fluorine); each of R⁶, R⁹ and R¹⁰ represent hydrogen, R⁷ represents C₁₋₆ alkoxy (such as methoxy) and R⁸ represents halogen (such as fluorine); each of R⁶, R⁹ and R¹⁰ represent hydrogen, R⁷ represents C₁₋₆ alkoxy (such as methoxy) and R⁸ represents C₁₋₆ alkyl (such as methyl); each of R⁶, R⁹ and R¹⁰ represent hydrogen, R⁷ represents halogen (such as chlorine) and R⁸ represents C₁₋₆ alkyl (such as methyl); both of R⁹ and R¹⁰ represent hydrogen, both of R⁶ and R⁸ represent halogen (such as fluorine) and R⁷ represents C₁₋₆ alkyl (such as methyl or ethyl); each of R⁷, R⁹ and R¹⁰ represent hydrogen and both of R⁶ and R⁸ represent halogen (such as chlorine, bromine or fluorine); both of R⁶ and R⁹ represent hydrogen and each of R⁷, R⁸ and R¹⁰ represent halogen (such as chlorine or fluorine); each of R⁸, R⁹ and R¹⁰ represent hydrogen and both of R⁶ and R⁷ represents halogen (such as chlorine or fluorine); each of R⁶, R⁹ and R¹⁰ represent hydrogen, R⁷ represents hydroxy and R⁸ represents halogen (such as fluorine); each of R⁶, R⁹ and R¹⁰ represent hydrogen, R⁷ represents C₂₋₆ alkenyl (such as ethenyl) and R⁸ represents halogen (such as fluorine) each of R⁶, R⁹ and R¹⁰ represent hydrogen, R⁷ represents hydroxy and R⁸ represents C₁₋₆ alkyl (such as methyl); each of R⁷, R⁸ and R¹⁰ represent hydrogen and both of R⁶ and R⁹ represent halogen (such as fluorine or chlorine); both of R⁷ and R¹⁰ represent hydrogen, both of R⁸ and R⁹ represent halogen (such as fluorine or chlorine) and R⁶ represents C₁₋₆ alkoxy (such as methoxy); both of R⁹ and R¹⁰ represent hydrogen and each of R⁶, R⁷ and R⁸ represent halogen (such as fluorine or chlorine); both of R⁹ and R¹⁰ represent hydrogen, both of R⁶ and R⁸ represent halogen (such as fluorine) and R⁷ represents C₂₋₆ alkenyl (such as ethenyl); each of R⁶, R⁹ and R¹⁰ represent hydrogen and R⁷ and R⁸ join to form a pyrrolinyl, tetrahydropyranyl, pyrazolyl or pyridyl ring optionally substituted by a methyl group; both of R⁷ and R¹⁰ represent hydrogen and each of R⁶, R⁸ and R⁹ represent halogen (such as chlorine or fluorine).

In one embodiment, each of R⁶, R⁹ and R¹⁰ represent hydrogen, R⁷ represents halogen (such as chlorine) and R⁸ represents C₃₋₈ cycloalkyl (such as cyclopropyl).

In one embodiment, Y represents —C(R⁶)═; and each of R⁶, R⁷, R⁸, R⁹ and R¹⁰ represent hydrogen.

In one embodiment, each of R⁶, R⁷, R⁸ and R¹⁰ represent hydrogen and R⁹ represents C₁₋₆ alkyl (such as methyl).

In one embodiment, each of R⁶, R⁷ and R¹⁰ represent hydrogen and R⁸ and R⁹ both represents halogen (such as fluorine or chlorine).

In one embodiment, each of R⁶, R⁷ and R¹⁰ represent hydrogen, R⁸ represents halogen (such as fluorine) and R⁹ represents C₁₋₆ alkyl (such as methyl).

In one embodiment, each of R⁶, R⁷ and R¹⁰ represent hydrogen, R⁸ represents halogen (such as fluorine) and R⁹ represents haloC₁₋₆ alkyl (such as fluoromethyl or trifluoromethyl).

In one embodiment, each of R⁶, R⁷, R⁸ and R¹⁰ represent hydrogen and R⁹ represents haloC₁₋₆ alkyl (such as fluoromethyl or difluoromethyl).

In one embodiment, each of R⁶, R⁷, R⁸ and R¹⁰ represent hydrogen and R⁹ represents halogen (such as chlorine).

In one embodiment, each of R⁶, R⁷ and R¹⁰ represent hydrogen, R⁸ represents halogen (such as fluorine) and R⁹ represents C₁₋₆ alkoxy (such as methoxy).

In one embodiment, each of R⁶, R⁸ and R¹⁰ represent hydrogen and R⁷ and R⁹ both represent halogen (such as fluorine or chlorine).

In one embodiment, each of R⁶, R⁷ and R¹⁰ represent hydrogen, R⁸ represents haloC₁₋₆ alkyl (such as trifluoromethyl) and R⁹ represents halogen (such as fluorine).

In one embodiment, each of R⁶, R⁸, R⁹ and R¹⁰ represent hydrogen and R⁷ represents C₁₋₆ alkyl (such as methyl, ethyl, CD₃, or C₂D₅); each of R⁶, R⁸, R⁹ and R¹⁰ represent hydrogen and R⁷ represents halogen (such as chlorine); each of R⁶, R⁹ and R¹⁰ represent hydrogen and R⁷ and R⁸ both represent halogen (such as fluorine, bromine or chlorine); each of R⁶, R⁹, and R¹⁰ represent hydrogen, R⁷ represents C₁₋₆ alkyl (such as methyl, ethyl, CD₃, or C₂D₅); each of R⁶, R⁸ and R¹⁰ represent hydrogen, R⁸ represents C₁₋₆ alkyl (such as methyl) and R⁹ represents halogen (such as chlorine); each of R⁶, R⁹ and R¹⁰ represent hydrogen, R⁷ represents C₁₋₆ alkyl (such as methyl or ethyl) and R⁸ represents halogen (such as chlorine or fluorine); each of R⁶, R⁹ and R¹⁰ represent hydrogen, R⁷ represents C₁₋₆ alkoxy (such as methoxy) and R⁸ represents halogen (such as fluorine); each of R⁶, R⁹ and R¹⁰ represent hydrogen, R⁷ represents C₁₋₆ alkoxy (such as methoxy) and R represents C₁₋₆ alkyl (such as methyl); each of R⁶, R⁹ and R¹⁰ represent hydrogen, R⁷ represents halogen (such as chlorine) and R⁸ represents C₁₋₆ alkyl (such as methyl); both of R⁹ and R¹⁰ represent hydrogen, both of R⁶ and R⁸ represent halogen (such as fluorine) and R⁷ represents C₁₋₆ alkyl (such as methyl or ethyl); each of R⁷, R⁹ and R¹⁰ represent hydrogen and both of R⁶ and R⁸ represent halogen (such as chlorine, bromine or fluorine); both of R⁶ and R⁹ represent hydrogen and each of R⁷, R⁸ and R¹⁰ represent halogen (such as chlorine or fluorine); each of R⁸, R⁹ and R¹⁰ represent hydrogen and both of R⁶ and R⁷ represents halogen (such as chlorine or fluorine); each of R⁶, R⁹ and R¹⁰ represent hydrogen, R¹ represents hydroxy and R⁸ represents halogen (such as fluorine); each of R⁶, R⁸ and R¹⁰ represent hydrogen, R⁷ represents C₂₋₆ alkenyl (such as ethenyl) and R⁸ represents halogen (such as fluorine) each of R⁶, R⁹ and R¹⁰ represent hydrogen, R⁷ represents hydroxy and R⁸ represents C₁₋₆ alkyl (such as methyl); each of R⁷, R⁸ and R¹⁰ represent hydrogen and both of R⁶ and R⁹ represent halogen (such as fluorine or chlorine); both of R⁷ and R¹⁰ represent hydrogen, both of R⁸ and R⁹ represent halogen (such as fluorine or chlorine) and R⁶ represents C₁₋₆ alkoxy (such as methoxy); both of R⁹ and R¹⁰ represent hydrogen and each of R⁶, R⁷ and R⁸ represent halogen (such as fluorine or chlorine); both of R⁹ and R¹⁰ represent hydrogen, both of R⁶ and R⁸ represent halogen (such as fluorine) and R⁷ represents C₂₋₆ alkenyl (such as ethenyl); each of R⁶, R⁹ and R¹⁰ represent hydrogen and R⁷ and R⁸ join to form a pyrrolinyl or tetrahydropyranyl ring.

In one embodiment, both of R⁷ and R¹⁰ represent hydrogen and each of R⁶, R⁸ and R⁹ represent halogen (such as chlorine or fluorine).

In one embodiment, Y represents —C(R⁶)═; and each of R⁶, R⁷, R¹, R⁹ and R¹⁰ represent hydrogen.

In one embodiment, each of R⁶, R⁷, R⁸ and R¹⁰ represent hydrogen and R⁹ represents C₁₋₆ alkyl (such as methyl).

In one embodiment, each of R⁶, R⁷ and R¹⁰ represent hydrogen and R⁸ and R⁹ both represents halogen (such as fluorine or chlorine).

In one embodiment, each of R⁶, R⁷ and R¹⁰ represent hydrogen, R⁸ represents halogen (such as fluorine) and R⁹ represents C₁₋₆ alkyl (such as methyl).

In one embodiment, each of R⁶, R⁷ and R¹⁰ represent hydrogen, R⁸ represents halogen (such as fluorine) and R¹ represents haloC₁₋₆ alkyl (such as fluoromethyl or trifluoromethyl).

In one embodiment, each of R⁶, R⁷, R⁸ and R¹⁰ represent hydrogen and R⁹ represents haloC₁₋₆ alkyl (such as fluoromethyl or difluoromethyl).

In one embodiment, each of R⁶, R⁷, R⁸ and R¹⁰ represent hydrogen and R⁹ represents halogen (such as chlorine).

In one embodiment, each of R⁶, R⁷ and R¹⁰ represent hydrogen, R⁸ represents halogen (such as fluorine) and R⁹ represents C₁₋₆ alkoxy (such as methoxy).

In one embodiment, each of R⁶, R⁸ and R¹⁰ represent hydrogen and R⁷ and R⁹ both represent halogen (such as fluorine).

In one embodiment, each of R⁶, R⁷ and R¹⁰ represent hydrogen, R⁸ represents haloC₁₋₆ alkyl (such as trifluoromethyl) and R⁹ represents halogen (such as fluorine).

In one embodiment, each of R⁶, R⁸, R⁹ and R¹⁰ represent hydrogen and R⁷ represents C₁₋₆ alkyl (such as methyl).

In one embodiment, each of R⁶, R⁹ and R¹⁰ represent hydrogen and R⁷ and R⁸ both represent halogen (such as fluorine or chlorine).

In one embodiment, Y represents —C(R⁶)═; and each of R⁶, R⁹ and R¹⁰ represent hydrogen and R⁷ and R⁸ both represent halogen (such as fluorine or chlorine), such as each of R⁶, R⁹ and R¹⁰ represent hydrogen, R⁷ represents halogen (such as chlorine) and R⁸ represents halogen (such as fluorine).

In an alternative embodiment, Y represents —N═; and R⁸ and R¹⁰ both represent hydrogen, R⁷ represents C₁₋₆ alkyl (such as methyl) and R⁹ represents haloC₁₋₆ alkyl (such as trifluoromethyl).

R⁷ and R⁸ join to form a pyrrolinyl ring and R⁹ and R¹⁰ both represent hydrogen.

R⁹ and R¹⁰ both represent hydrogen, R⁷ represents C₁₋₆ alkyl (such as methyl) and R⁸ represents halogen (such as fluorine); each of R⁸, R⁹ and R¹⁰ represent hydrogen and R⁷ represents —NR^{m}Rⁿ (such as —NH₂, —NHMe or —NMe₂).

R⁹ and R¹⁰ both represent hydrogen and R⁷ and R⁸ join to form a pyrrolinyl, morpholinyl, furanyl or thiophenyl ring optionally substituted by a methyl, fluorine or chlorine group.

In one embodiment, R¹⁰ represents hydrogen, R⁹ represents halogen (such as chlorine) and R⁷ and R⁸ join to form a pyrrolinyl ring optionally substituted by a methyl group.

In one embodiment, Y represents —N═; and R⁸ and R¹⁰ both represent hydrogen, R⁷ represents C₁₋₆ alkyl (such as methyl) and R⁹ represents haloC₁₋₆ alkyl (such as trifluoromethyl).

In one embodiment, Y represents —N═; and R⁹ and R¹⁰ both represent hydrogen, R⁷ represents C₁₋₆ alkyl (such as methyl, ethyl, CD₃, or C₂D₅), and R⁸ represents halogen (such as fluorine).

In one embodiment, Y represents —N═; and R⁹ and R¹⁰ both represent hydrogen, and both R⁷ and R⁸ represent halogen (such as fluorine or chlorine).

In one embodiment, R⁷ and R⁸ join to form a pyrrolinyl ring and R⁹ and R¹⁰ both represent hydrogen.

In one embodiment, Y represents —N═; and R⁸ and R¹⁰ both represent hydrogen, R⁷ represents C₁₋₆ alkyl (such as methyl) and R⁹ represents haloC₁₋₆ alkyl (such as trifluoromethyl).

In one embodiment, the compound of Formula (I) is represented by one of the following compounds:

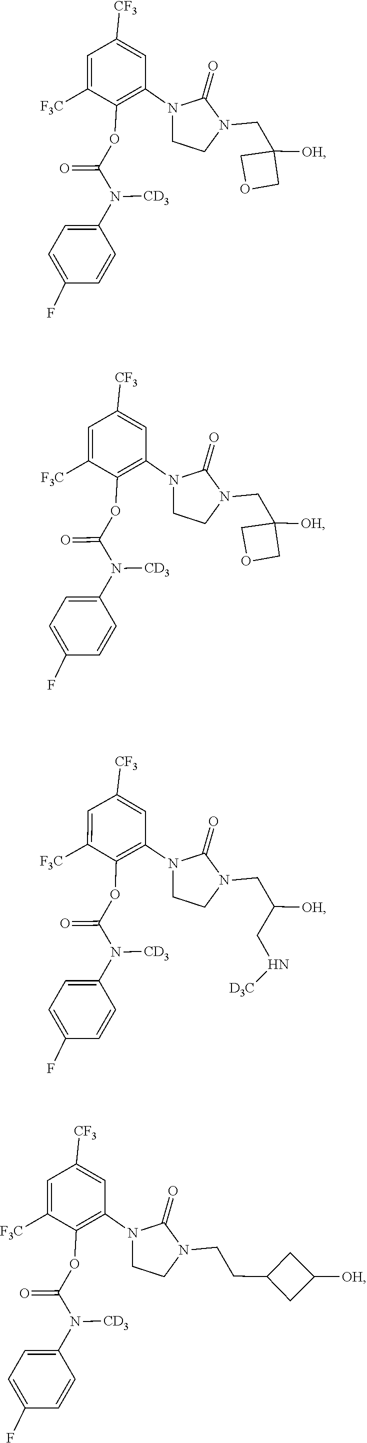 [SMILES: [2H]C([2H])([2H])N(C(=O)Oc1c(N2CCN(CC3(O)COC3)C2=O)cc(C(F)(F)F)cc1C(F)(F)F)c1ccc(F)cc1 ; [2H]C([2H])([2H])N(C(=O)Oc1c(N2CCN(CC3(O)COC3)C2=O)cc(C(F)(F)F)cc1C(F)(F)F)c1ccc(F)cc1 ; [2H]C([2H])([2H])NCC(O)CN1CCN(c2cc(C(F)(F)F)cc(C(F)(F)F)c2OC(=O)N(c2ccc(F)cc2)C([2H])([2H])[2H])C1=O ; [2H]C([2H])([2H])N(C(=O)Oc1c(N2CCN(CCC3CC(O)C3)C2=O)cc(C(F)(F)F)cc1C(F)(F)F)c1ccc(F)cc1]

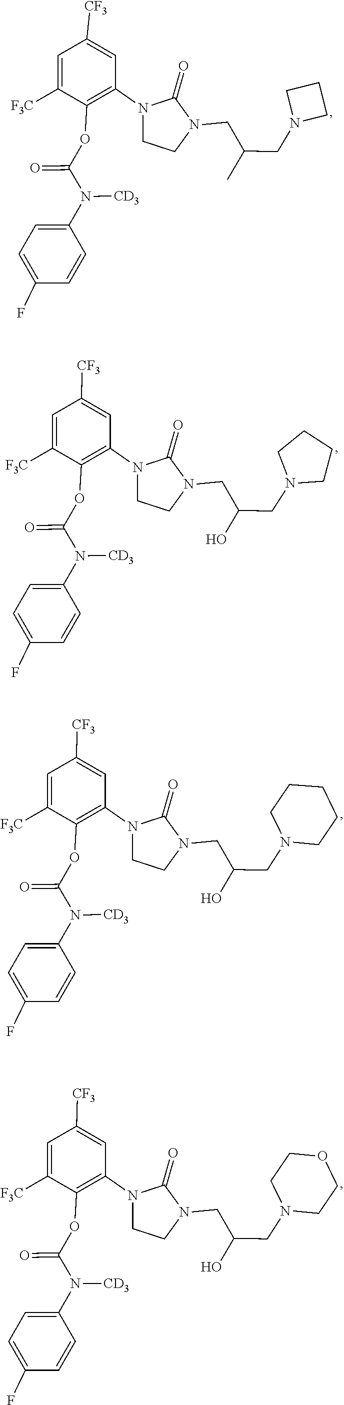 [SMILES: [2H]C([2H])([2H])N(C(=O)Oc1c(N2CCN(CC(C)CN3CCC3)C2=O)cc(C(F)(F)F)cc1C(F)(F)F)c1ccc(F)cc1 ; [2H]C([2H])([2H])N(C(=O)Oc1c(N2CCN(CC(O)CN3CCCC3)C2=O)cc(C(F)(F)F)cc1C(F)(F)F)c1ccc(F)cc1 ; [2H]C([2H])([2H])N(C(=O)Oc1c(N2CCN(CC(O)CN3CCCCC3)C2=O)cc(C(F)(F)F)cc1C(F)(F)F)c1ccc(F)cc1 ; [2H]C([2H])([2H])N(C(=O)Oc1c(N2CCN(CC(O)CN3CCOCC3)C2=O)cc(C(F)(F)F)cc1C(F)(F)F)c1ccc(F)cc1]

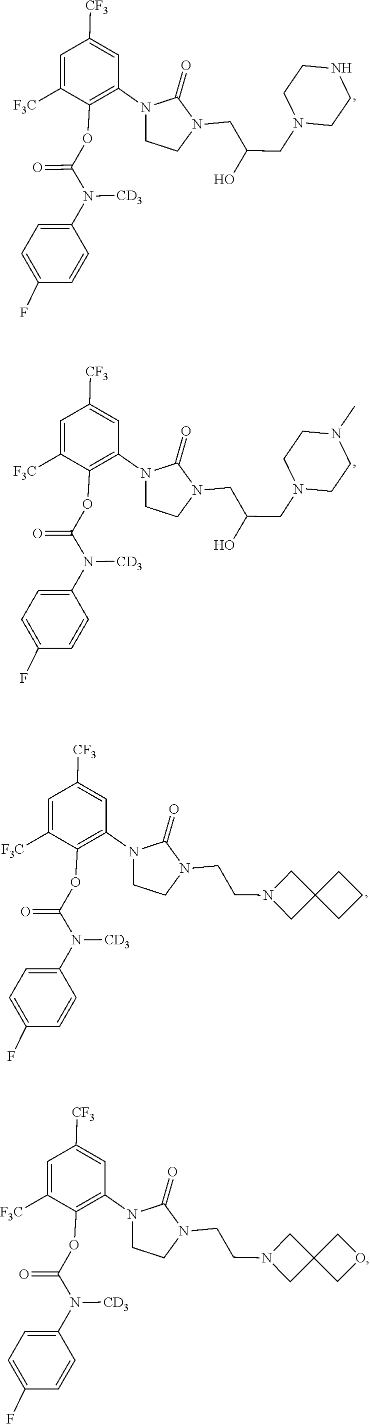 [SMILES: [2H]C([2H])([2H])N(C(=O)Oc1c(N2CCN(CC(O)CN3CCNCC3)C2=O)cc(C(F)(F)F)cc1C(F)(F)F)c1ccc(F)cc1 ; [2H]C([2H])([2H])N(C(=O)Oc1c(N2CCN(CC(O)CN3CCN(C)CC3)C2=O)cc(C(F)(F)F)cc1C(F)(F)F)c1ccc(F)cc1 ; [2H]C([2H])([2H])N(C(=O)Oc1c(N2CCN(CCN3CC4(CCC4)C3)C2=O)cc(C(F)(F)F)cc1C(F)(F)F)c1ccc(F)cc1 ; [2H]C([2H])([2H])N(C(=O)Oc1c(N2CCN(CCN3CC4(COC4)C3)C2=O)cc(C(F)(F)F)cc1C(F)(F)F)c1ccc(F)cc1]

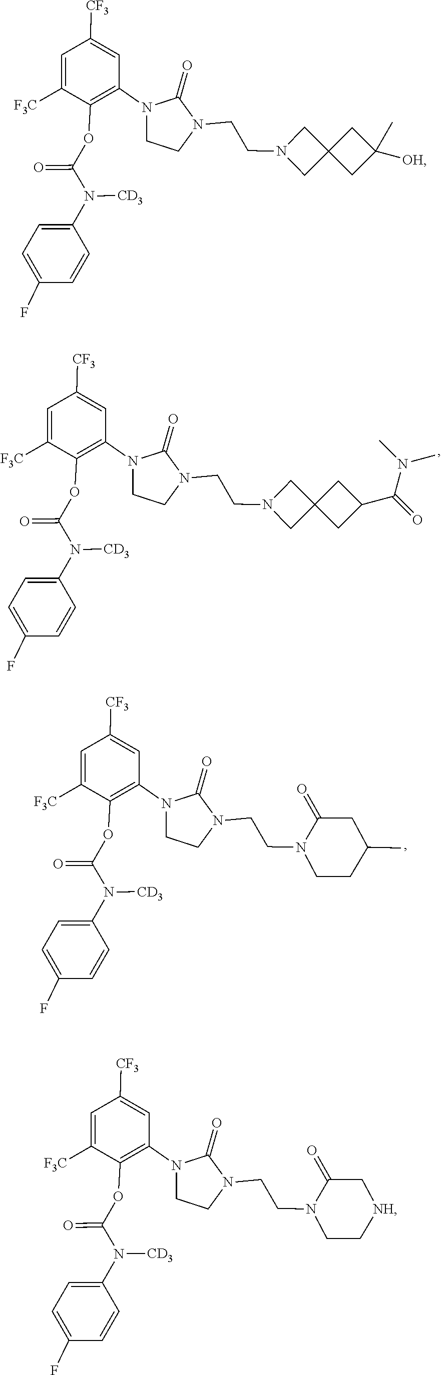 [SMILES: [2H]C([2H])([2H])N(C(=O)Oc1c(N2CCN(CCN3CC4(C3)CC(C)(O)C4)C2=O)cc(C(F)(F)F)cc1C(F)(F)F)c1ccc(F)cc1 ; [2H]C([2H])([2H])N(C(=O)Oc1c(N2CCN(CCN3CC4(CC(C(=O)N(C)C)C4)C3)C2=O)cc(C(F)(F)F)cc1C(F)(F)F)c1ccc(F)cc1 ; [2H]C([2H])([2H])N(C(=O)Oc1c(N2CCN(CCN3CCC(C)CC3=O)C2=O)cc(C(F)(F)F)cc1C(F)(F)F)c1ccc(F)cc1 ; [2H]C([2H])([2H])N(C(=O)Oc1c(N2CCN(CCN3CCNCC3=O)C2=O)cc(C(F)(F)F)cc1C(F)(F)F)c1ccc(F)cc1]

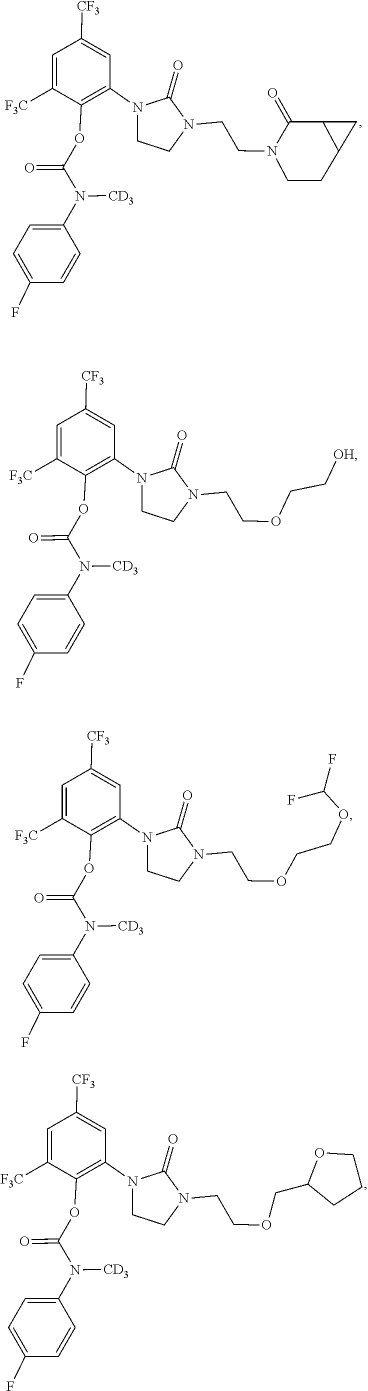 [SMILES: [2H]C([2H])([2H])N(C(=O)Oc1c(N2CCN(CCN3CCC4CC4C3=O)C2=O)cc(C(F)(F)F)cc1C(F)(F)F)c1ccc(F)cc1 ; [2H]C([2H])([2H])N(C(=O)Oc1c(N2CCN(CCOCCO)C2=O)cc(C(F)(F)F)cc1C(F)(F)F)c1ccc(F)cc1 ; [2H]C([2H])([2H])N(C(=O)Oc1c(N2CCN(CCOCCOC(F)F)C2=O)cc(C(F)(F)F)cc1C(F)(F)F)c1ccc(F)cc1 ; [2H]C([2H])([2H])N(C(=O)Oc1c(N2CCN(CCOCC3CCCO3)C2=O)cc(C(F)(F)F)cc1C(F)(F)F)c1ccc(F)cc1]

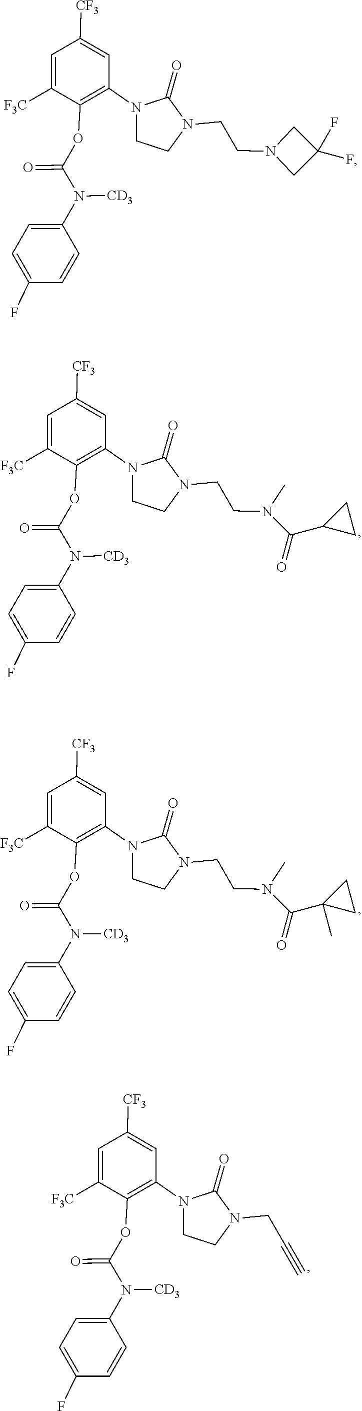 [SMILES: [2H]C([2H])([2H])N(C(=O)Oc1c(N2CCN(CCN3CC(F)(F)C3)C2=O)cc(C(F)(F)F)cc1C(F)(F)F)c1ccc(F)cc1 ; [2H]C([2H])([2H])N(C(=O)Oc1c(N2CCN(CCN(C)C(=O)C3CC3)C2=O)cc(C(F)(F)F)cc1C(F)(F)F)c1ccc(F)cc1 ; [2H]C([2H])([2H])N(C(=O)Oc1c(N2CCN(CCN(C)C(=O)C3(C)CC3)C2=O)cc(C(F)(F)F)cc1C(F)(F)F)c1ccc(F)cc1 ; [2H]C([2H])([2H])N(C(=O)Oc1c(N2CCN(CC#C)C2=O)cc(C(F)(F)F)cc1C(F)(F)F)c1ccc(F)cc1]

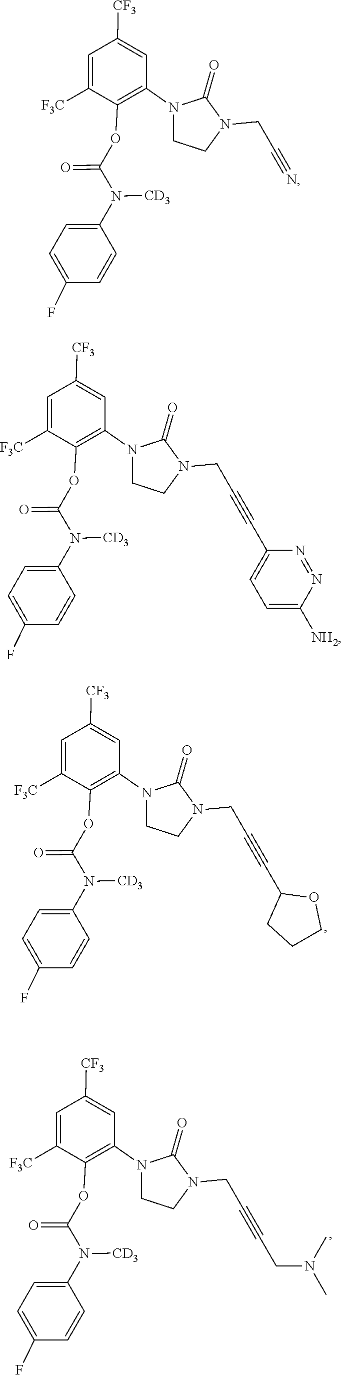

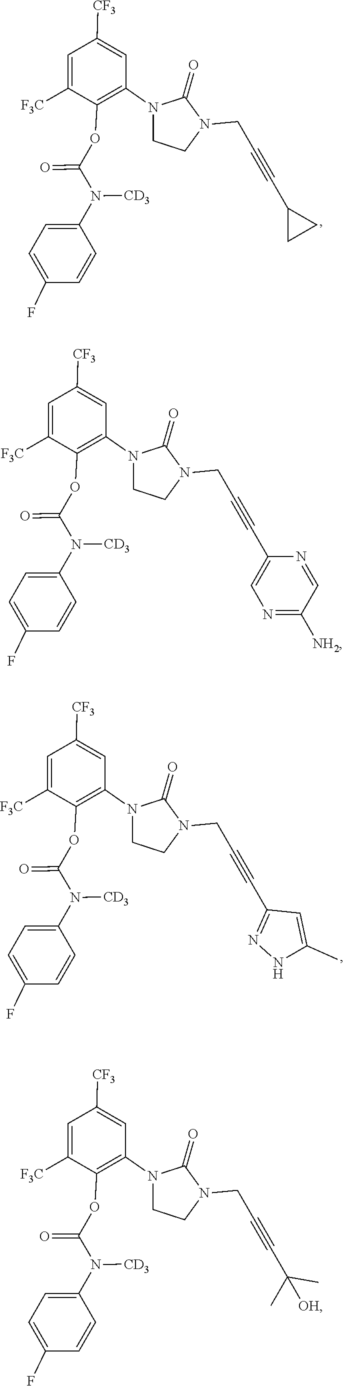 [SMILES: [2H]C([2H])([2H])N(C(=O)Oc1c(N2CCN(CC#CC3CC3)C2=O)cc(C(F)(F)F)cc1C(F)(F)F)c1ccc(F)cc1 ; [2H]C([2H])([2H])N(C(=O)Oc1c(N2CCN(CC#Cc3cnc(N)cn3)C2=O)cc(C(F)(F)F)cc1C(F)(F)F)c1ccc(F)cc1 ; [2H]C([2H])([2H])N(C(=O)Oc1c(N2CCN(CC#Cc3cc(C)[nH]n3)C2=O)cc(C(F)(F)F)cc1C(F)(F)F)c1ccc(F)cc1 ; [2H]C([2H])([2H])N(C(=O)Oc1c(N2CCN(CC#CC(C)(C)O)C2=O)cc(C(F)(F)F)cc1C(F)(F)F)c1ccc(F)cc1]

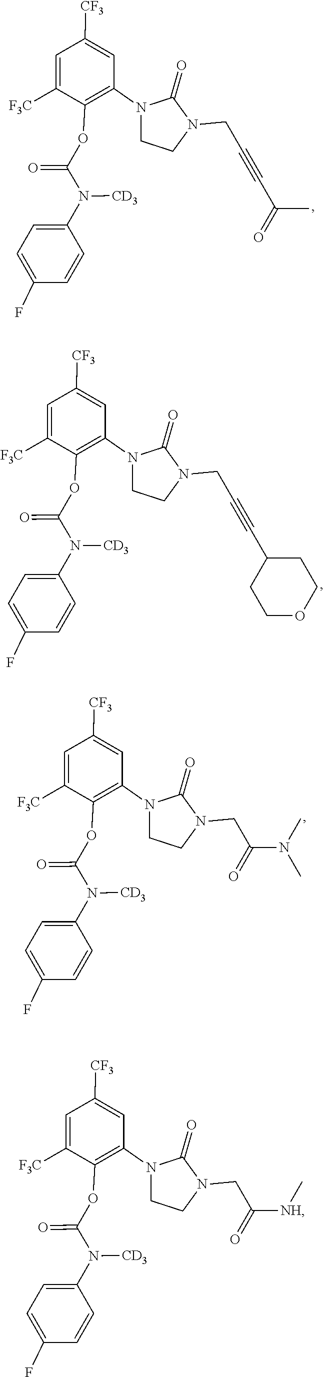 [SMILES: [2H]C([2H])([2H])N(C(=O)Oc1c(N2CCN(CC#CC(C)=O)C2=O)cc(C(F)(F)F)cc1C(F)(F)F)c1ccc(F)cc1 ; [2H]C([2H])([2H])N(C(=O)Oc1c(N2CCN(CC#CC3CCOCC3)C2=O)cc(C(F)(F)F)cc1C(F)(F)F)c1ccc(F)cc1 ; [2H]C([2H])([2H])N(C(=O)Oc1c(N2CCN(CC(=O)N(C)C)C2=O)cc(C(F)(F)F)cc1C(F)(F)F)c1ccc(F)cc1 ; [2H]C([2H])([2H])N(C(=O)Oc1c(N2CCN(CC(=O)NC)C2=O)cc(C(F)(F)F)cc1C(F)(F)F)c1ccc(F)cc1]

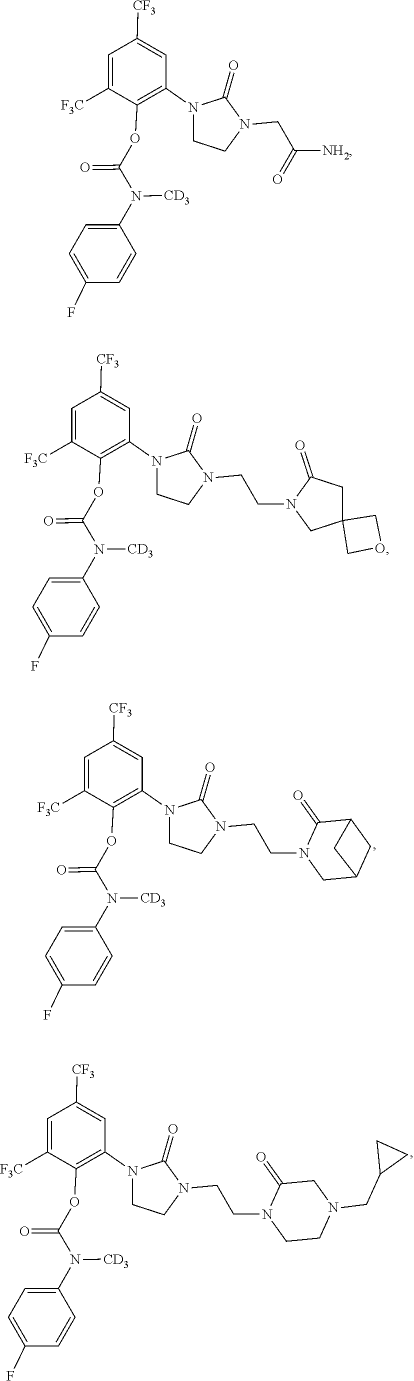 [SMILES: [2H]C([2H])([2H])N(C(=O)Oc1c(N2CCN(CC(N)=O)C2=O)cc(C(F)(F)F)cc1C(F)(F)F)c1ccc(F)cc1 ; [2H]C([2H])([2H])N(C(=O)Oc1c(N2CCN(CCN3CC4(COC4)CC3=O)C2=O)cc(C(F)(F)F)cc1C(F)(F)F)c1ccc(F)cc1 ; [2H]C([2H])([2H])N(C(=O)Oc1c(N2CCN(CCN3CC4CC(C4)C3=O)C2=O)cc(C(F)(F)F)cc1C(F)(F)F)c1ccc(F)cc1 ; [2H]C([2H])([2H])N(C(=O)Oc1c(N2CCN(CCN3CCN(CC4CC4)CC3=O)C2=O)cc(C(F)(F)F)cc1C(F)(F)F)c1ccc(F)cc1]

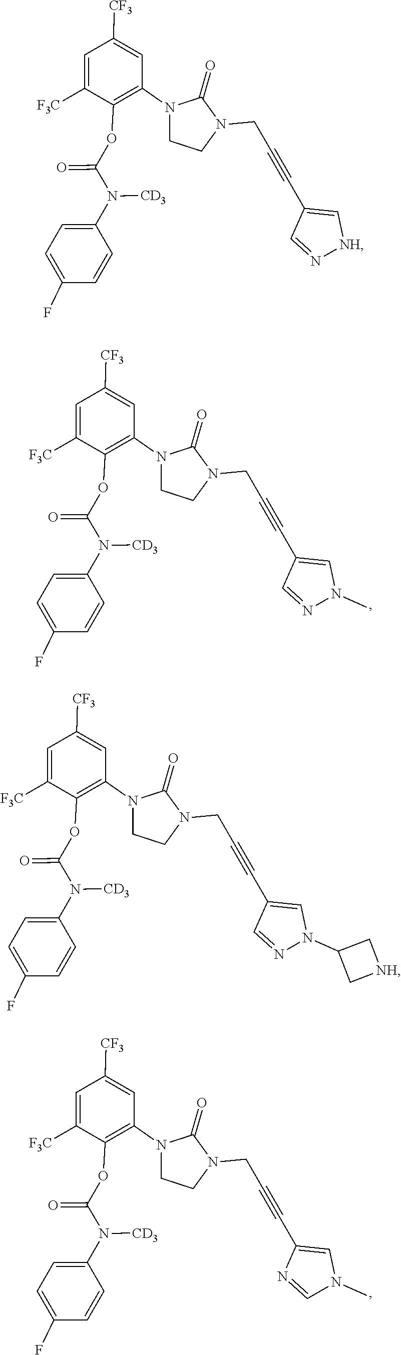 [SMILES: [2H]C([2H])([2H])N(C(=O)Oc1c(N2CCN(CC#Cc3cn[nH]c3)C2=O)cc(C(F)(F)F)cc1C(F)(F)F)c1ccc(F)cc1 ; [2H]C([2H])([2H])N(C(=O)Oc1c(N2CCN(CC#Cc3cnn(C)c3)C2=O)cc(C(F)(F)F)cc1C(F)(F)F)c1ccc(F)cc1 ; [2H]C([2H])([2H])N(C(=O)Oc1c(N2CCN(CC#Cc3cnn(C4CNC4)c3)C2=O)cc(C(F)(F)F)cc1C(F)(F)F)c1ccc(F)cc1 ; [2H]C([2H])([2H])N(C(=O)Oc1c(N2CCN(CC#Cc3cn(C)cn3)C2=O)cc(C(F)(F)F)cc1C(F)(F)F)c1ccc(F)cc1]

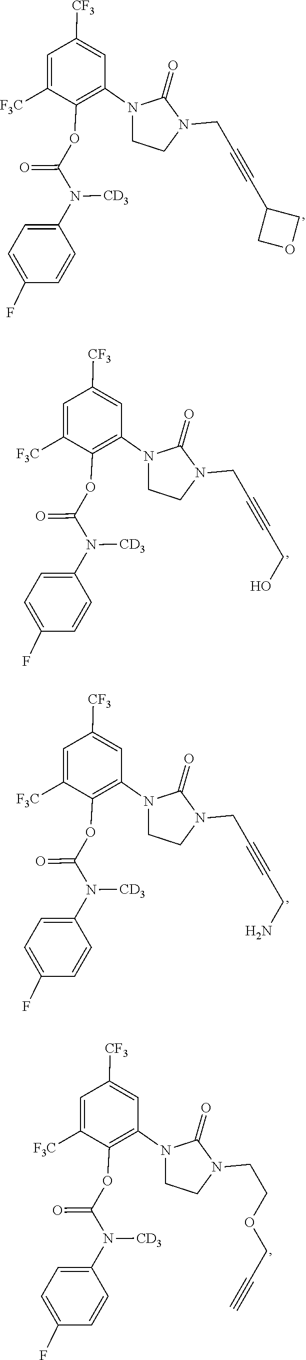 [SMILES: [2H]C([2H])([2H])N(C(=O)Oc1c(N2CCN(CC#CC3COC3)C2=O)cc(C(F)(F)F)cc1C(F)(F)F)c1ccc(F)cc1 ; [2H]C([2H])([2H])N(C(=O)Oc1c(N2CCN(CC#CCO)C2=O)cc(C(F)(F)F)cc1C(F)(F)F)c1ccc(F)cc1 ; [2H]C([2H])([2H])N(C(=O)Oc1c(N2CCN(CC#CCN)C2=O)cc(C(F)(F)F)cc1C(F)(F)F)c1ccc(F)cc1 ; [2H]C([2H])([2H])N(C(=O)Oc1c(N2CCN(CCOCC#C)C2=O)cc(C(F)(F)F)cc1C(F)(F)F)c1ccc(F)cc1]

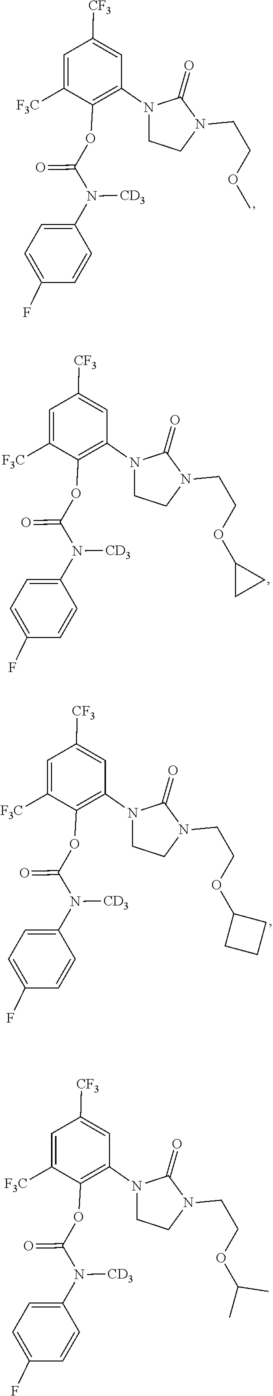 [SMILES: [2H]C([2H])([2H])N(C(=O)Oc1c(N2CCN(CCOC)C2=O)cc(C(F)(F)F)cc1C(F)(F)F)c1ccc(F)cc1 ; [2H]C([2H])([2H])N(C(=O)Oc1c(N2CCN(CCOC3CC3)C2=O)cc(C(F)(F)F)cc1C(F)(F)F)c1ccc(F)cc1 ; [2H]C([2H])([2H])N(C(=O)Oc1c(N2CCN(CCOC3CCC3)C2=O)cc(C(F)(F)F)cc1C(F)(F)F)c1ccc(F)cc1 ; [2H]C([2H])([2H])N(C(=O)Oc1c(N2CCN(CCOC(C)C)C2=O)cc(C(F)(F)F)cc1C(F)(F)F)c1ccc(F)cc1]

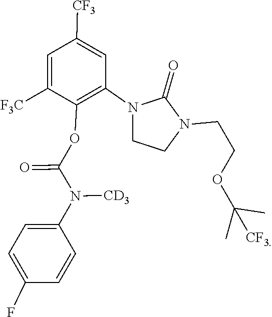 [SMILES: [2H]C([2H])([2H])N(C(=O)Oc1c(N2CCN(CCOC(C)(C)C(F)(F)F)C2=O)cc(C(F)(F)F)cc1C(F)(F)F)c1ccc(F)cc1]

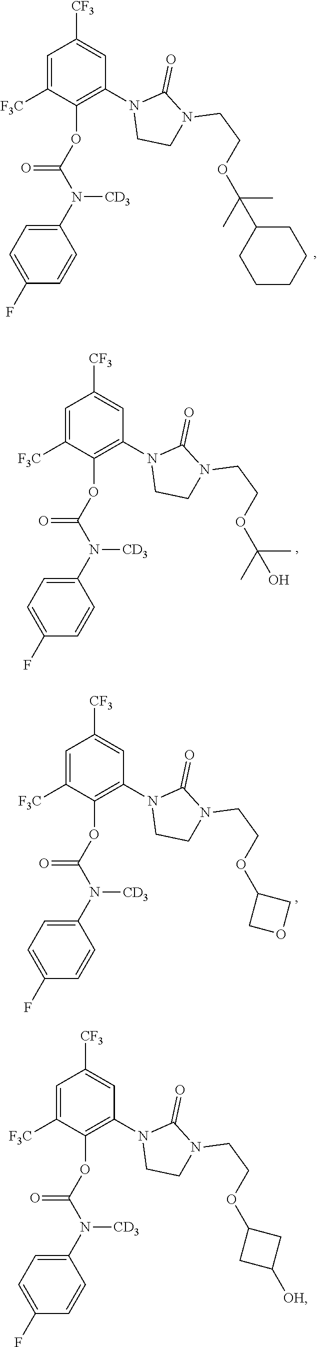 [SMILES: [2H]C([2H])([2H])N(C(=O)Oc1c(N2CCN(CCOC(C)(C)C3CCCCC3)C2=O)cc(C(F)(F)F)cc1C(F)(F)F)c1ccc(F)cc1 ; [2H]C([2H])([2H])N(C(=O)Oc1c(N2CCN(CCOC(C)(C)O)C2=O)cc(C(F)(F)F)cc1C(F)(F)F)c1ccc(F)cc1 ; [2H]C([2H])([2H])N(C(=O)Oc1c(N2CCN(CCOC3COC3)C2=O)cc(C(F)(F)F)cc1C(F)(F)F)c1ccc(F)cc1 ; [2H]C([2H])([2H])N(C(=O)Oc1c(N2CCN(CCOC3CC(O)C3)C2=O)cc(C(F)(F)F)cc1C(F)(F)F)c1ccc(F)cc1]

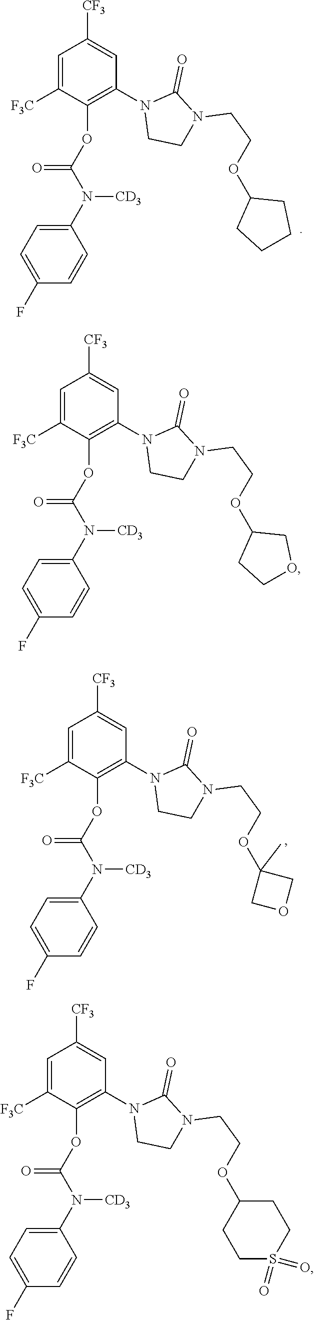 [SMILES: [2H]C([2H])([2H])N(C(=O)Oc1c(N2CCN(CCOC3CCCC3)C2=O)cc(C(F)(F)F)cc1C(F)(F)F)c1ccc(F)cc1 ; [2H]C([2H])([2H])N(C(=O)Oc1c(N2CCN(CCOC3CCOC3)C2=O)cc(C(F)(F)F)cc1C(F)(F)F)c1ccc(F)cc1 ; [2H]C([2H])([2H])N(C(=O)Oc1c(N2CCN(CCOC3(C)COC3)C2=O)cc(C(F)(F)F)cc1C(F)(F)F)c1ccc(F)cc1 ; [2H]C([2H])([2H])N(C(=O)Oc1c(N2CCN(CCOC3CCS(=O)(=O)CC3)C2=O)cc(C(F)(F)F)cc1C(F)(F)F)c1ccc(F)cc1]

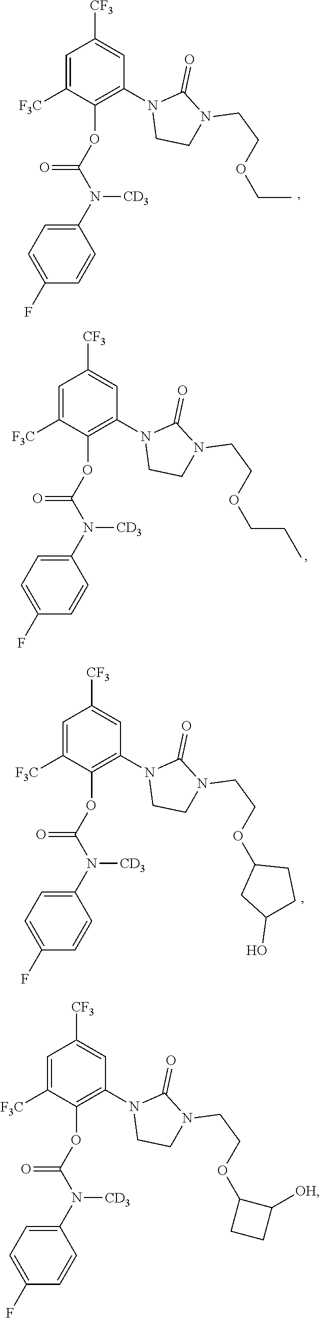 [SMILES: [2H]C([2H])([2H])N(C(=O)Oc1c(N2CCN(CCOCC)C2=O)cc(C(F)(F)F)cc1C(F)(F)F)c1ccc(F)cc1 ; [2H]C([2H])([2H])N(C(=O)Oc1c(N2CCN(CCOCCC)C2=O)cc(C(F)(F)F)cc1C(F)(F)F)c1ccc(F)cc1 ; [2H]C([2H])([2H])N(C(=O)Oc1c(N2CCN(CCOC3CCC(O)C3)C2=O)cc(C(F)(F)F)cc1C(F)(F)F)c1ccc(F)cc1 ; [2H]C([2H])([2H])N(C(=O)Oc1c(N2CCN(CCOC3CCC3O)C2=O)cc(C(F)(F)F)cc1C(F)(F)F)c1ccc(F)cc1]

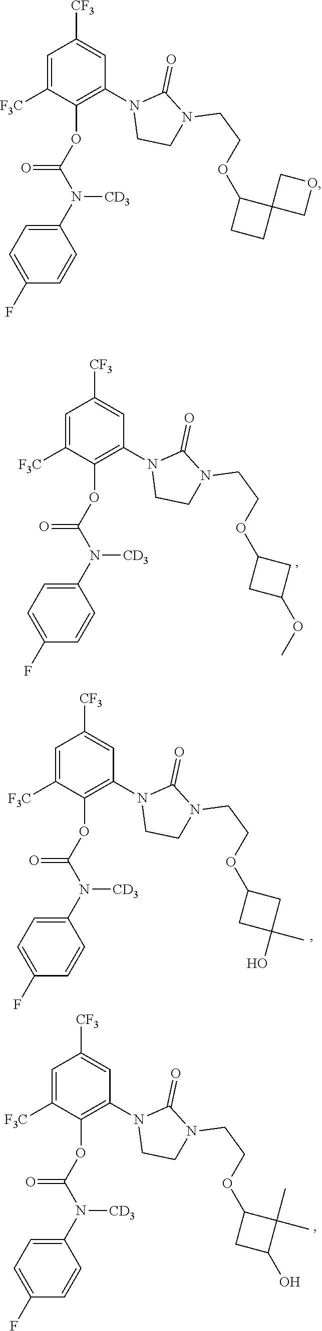

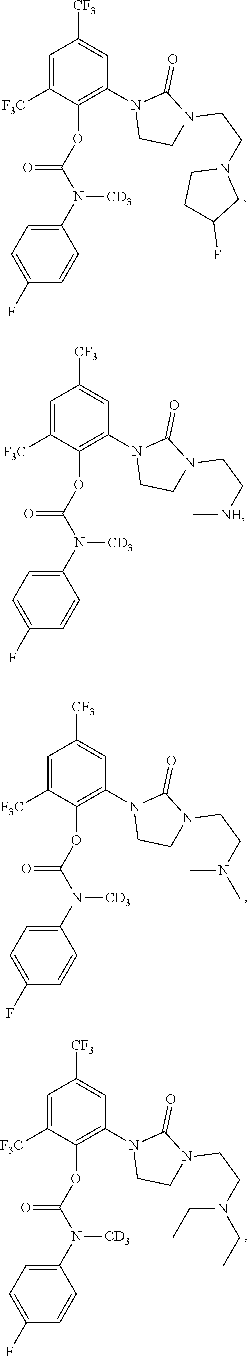 [SMILES: [2H]C([2H])([2H])N(C(=O)Oc1c(N2CCN(CCN3CCC(F)C3)C2=O)cc(C(F)(F)F)cc1C(F)(F)F)c1ccc(F)cc1 ; [2H]C([2H])([2H])N(C(=O)Oc1c(N2CCN(CCNC)C2=O)cc(C(F)(F)F)cc1C(F)(F)F)c1ccc(F)cc1 ; [2H]C([2H])([2H])N(C(=O)Oc1c(N2CCN(CCN(C)C)C2=O)cc(C(F)(F)F)cc1C(F)(F)F)c1ccc(F)cc1 ; [2H]C([2H])([2H])N(C(=O)Oc1c(N2CCN(CCN(CC)CC)C2=O)cc(C(F)(F)F)cc1C(F)(F)F)c1ccc(F)cc1]

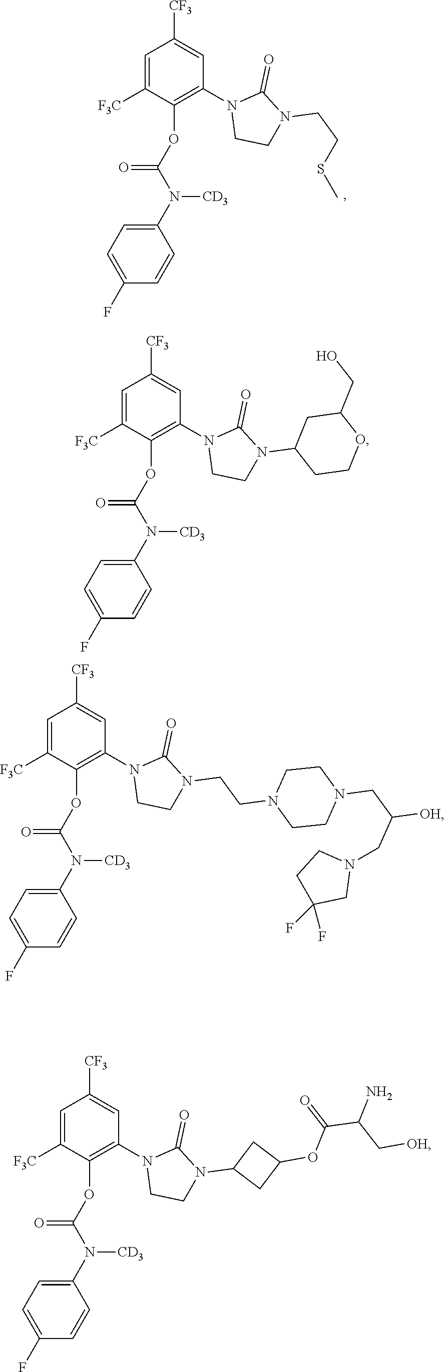

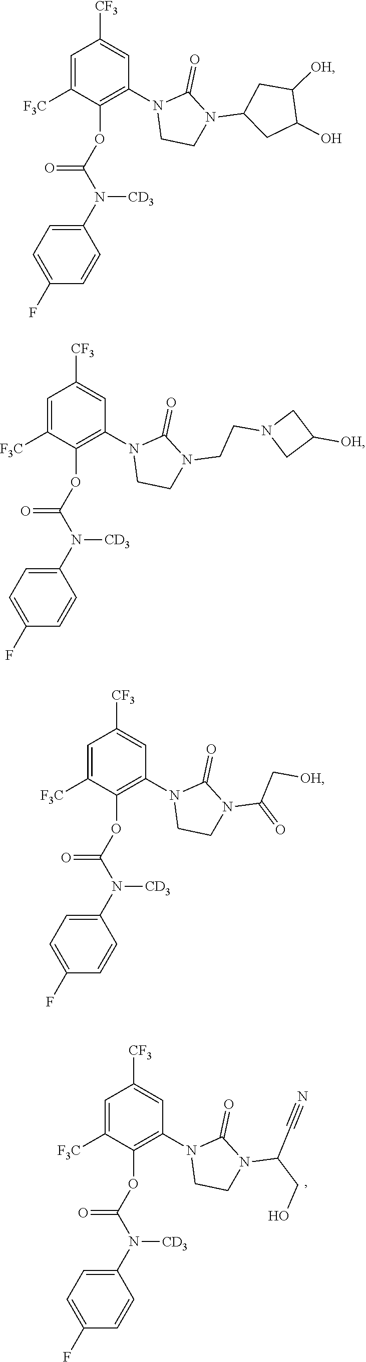 [SMILES: [2H]C([2H])([2H])N(C(=O)Oc1c(N2CCN(C3CC(O)C(O)C3)C2=O)cc(C(F)(F)F)cc1C(F)(F)F)c1ccc(F)cc1 ; [2H]C([2H])([2H])N(C(=O)Oc1c(N2CCN(CCN3CC(O)C3)C2=O)cc(C(F)(F)F)cc1C(F)(F)F)c1ccc(F)cc1 ; [2H]C([2H])([2H])N(C(=O)Oc1c(N2CCN(C(=O)CO)C2=O)cc(C(F)(F)F)cc1C(F)(F)F)c1ccc(F)cc1 ; [2H]C([2H])([2H])N(C(=O)Oc1c(N2CCN(C(C#N)CO)C2=O)cc(C(F)(F)F)cc1C(F)(F)F)c1ccc(F)cc1]

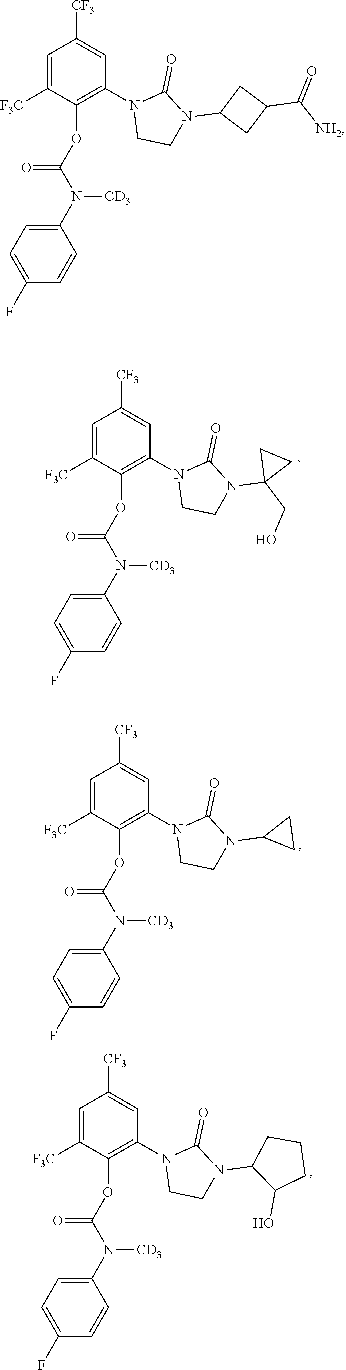 [SMILES: [2H]C([2H])([2H])N(C(=O)Oc1c(N2CCN(C3CC(C(N)=O)C3)C2=O)cc(C(F)(F)F)cc1C(F)(F)F)c1ccc(F)cc1 ; [2H]C([2H])([2H])N(C(=O)Oc1c(N2CCN(C3(CO)CC3)C2=O)cc(C(F)(F)F)cc1C(F)(F)F)c1ccc(F)cc1 ; [2H]C([2H])([2H])N(C(=O)Oc1c(N2CCN(C3CC3)C2=O)cc(C(F)(F)F)cc1C(F)(F)F)c1ccc(F)cc1 ; [2H]C([2H])([2H])N(C(=O)Oc1c(N2CCN(C3CCCC3O)C2=O)cc(C(F)(F)F)cc1C(F)(F)F)c1ccc(F)cc1]

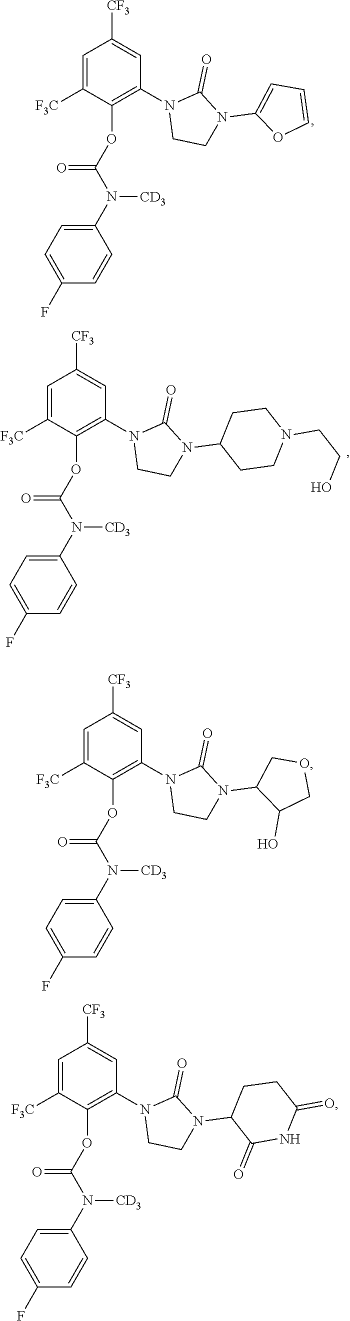

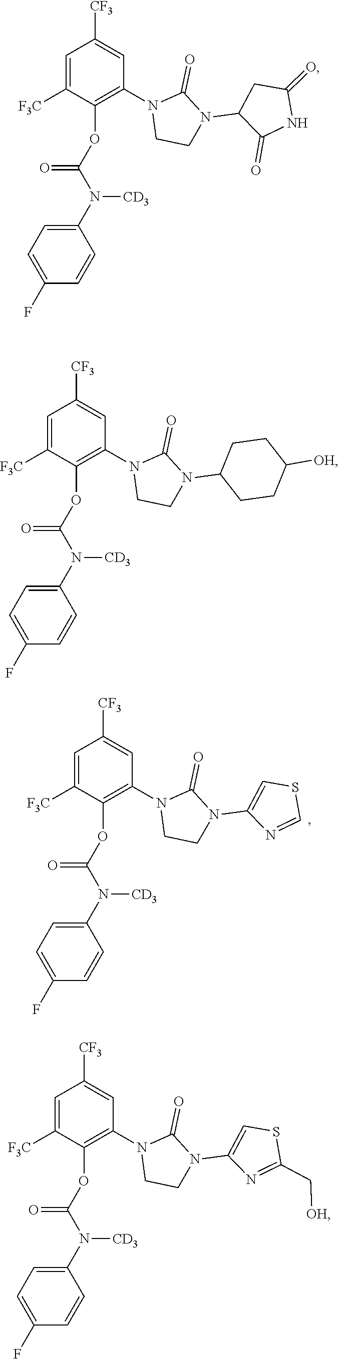 [SMILES: [2H]C([2H])([2H])N(C(=O)Oc1c(N2CCN(C3CC(=O)NC3=O)C2=O)cc(C(F)(F)F)cc1C(F)(F)F)c1ccc(F)cc1 ; [2H]C([2H])([2H])N(C(=O)Oc1c(N2CCN(C3CCC(O)CC3)C2=O)cc(C(F)(F)F)cc1C(F)(F)F)c1ccc(F)cc1 ; [2H]C([2H])([2H])N(C(=O)Oc1c(N2CCN(c3cscn3)C2=O)cc(C(F)(F)F)cc1C(F)(F)F)c1ccc(F)cc1 ; [2H]C([2H])([2H])N(C(=O)Oc1c(N2CCN(c3csc(CO)n3)C2=O)cc(C(F)(F)F)cc1C(F)(F)F)c1ccc(F)cc1]

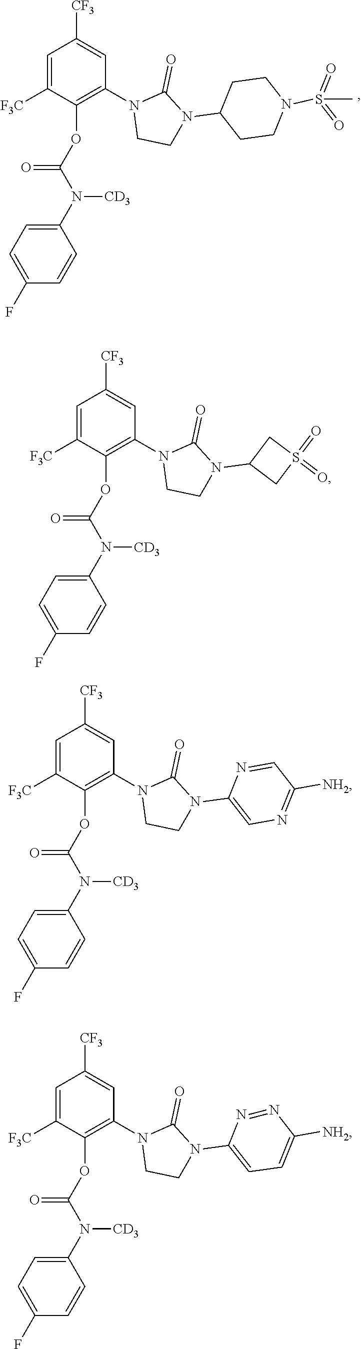 [SMILES: [2H]C([2H])([2H])N(C(=O)Oc1c(N2CCN(C3CCN(S(C)(=O)=O)CC3)C2=O)cc(C(F)(F)F)cc1C(F)(F)F)c1ccc(F)cc1 ; [2H]C([2H])([2H])N(C(=O)Oc1c(N2CCN(C3CS(=O)(=O)C3)C2=O)cc(C(F)(F)F)cc1C(F)(F)F)c1ccc(F)cc1 ; [2H]C([2H])([2H])N(C(=O)Oc1c(N2CCN(c3cnc(N)cn3)C2=O)cc(C(F)(F)F)cc1C(F)(F)F)c1ccc(F)cc1 ; [2H]C([2H])([2H])N(C(=O)Oc1c(N2CCN(c3ccc(N)nn3)C2=O)cc(C(F)(F)F)cc1C(F)(F)F)c1ccc(F)cc1]

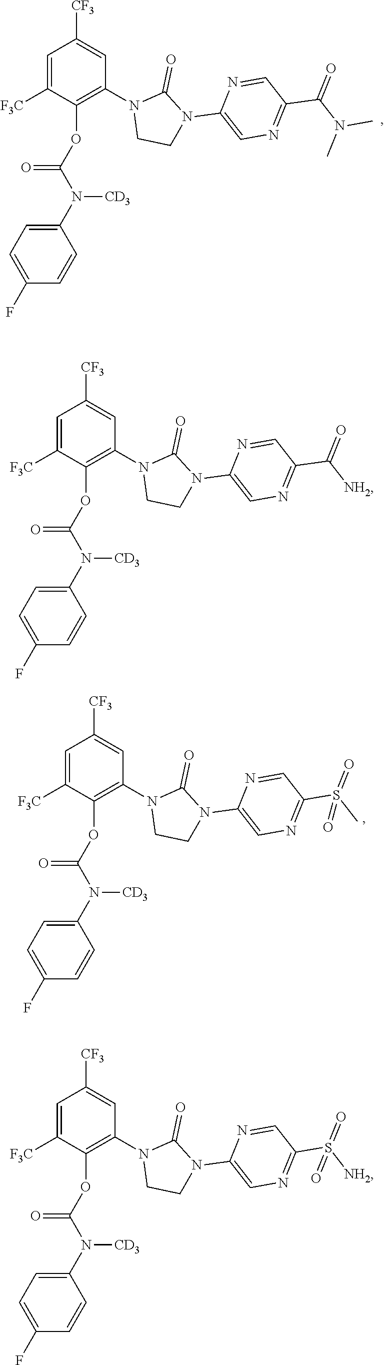 [SMILES: [2H]C([2H])([2H])N(C(=O)Oc1c(N2CCN(c3cnc(C(=O)N(C)C)cn3)C2=O)cc(C(F)(F)F)cc1C(F)(F)F)c1ccc(F)cc1 ; [2H]C([2H])([2H])N(C(=O)Oc1c(N2CCN(c3cnc(C(N)=O)cn3)C2=O)cc(C(F)(F)F)cc1C(F)(F)F)c1ccc(F)cc1 ; [2H]C([2H])([2H])N(C(=O)Oc1c(N2CCN(c3cnc(S(C)(=O)=O)cn3)C2=O)cc(C(F)(F)F)cc1C(F)(F)F)c1ccc(F)cc1 ; [2H]C([2H])([2H])N(C(=O)Oc1c(N2CCN(c3cnc(S(N)(=O)=O)cn3)C2=O)cc(C(F)(F)F)cc1C(F)(F)F)c1ccc(F)cc1]

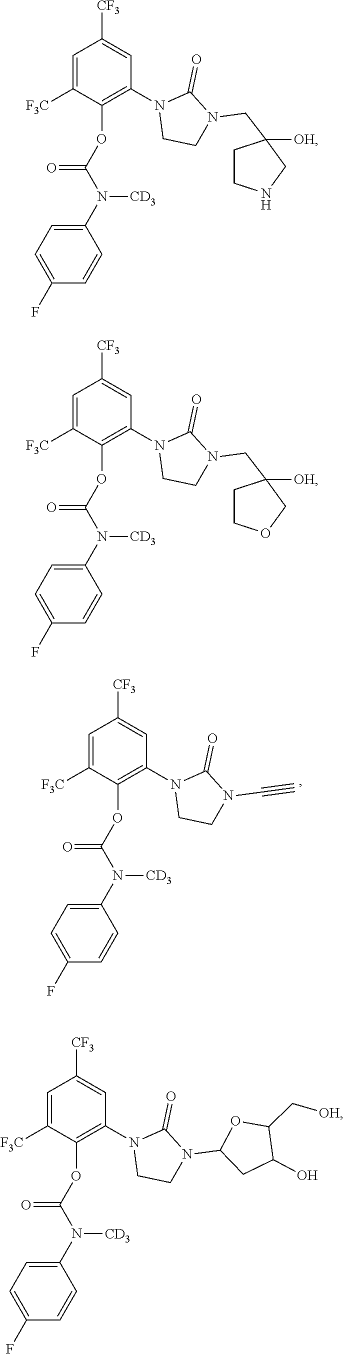 [SMILES: [2H]C([2H])([2H])N(C(=O)Oc1c(N2CCN(CC3(O)CCNC3)C2=O)cc(C(F)(F)F)cc1C(F)(F)F)c1ccc(F)cc1 ; [2H]C([2H])([2H])N(C(=O)Oc1c(N2CCN(CC3(O)CCOC3)C2=O)cc(C(F)(F)F)cc1C(F)(F)F)c1ccc(F)cc1 ; [2H]C([2H])([2H])N(C(=O)Oc1c(N2CCN(C#C)C2=O)cc(C(F)(F)F)cc1C(F)(F)F)c1ccc(F)cc1 ; [2H]C([2H])([2H])N(C(=O)Oc1c(N2CCN(C3CC(O)C(CO)O3)C2=O)cc(C(F)(F)F)cc1C(F)(F)F)c1ccc(F)cc1]

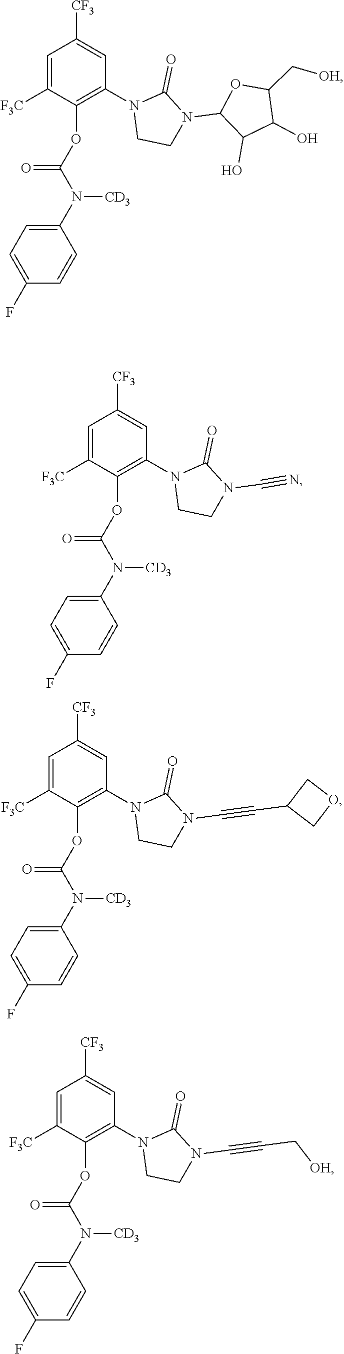

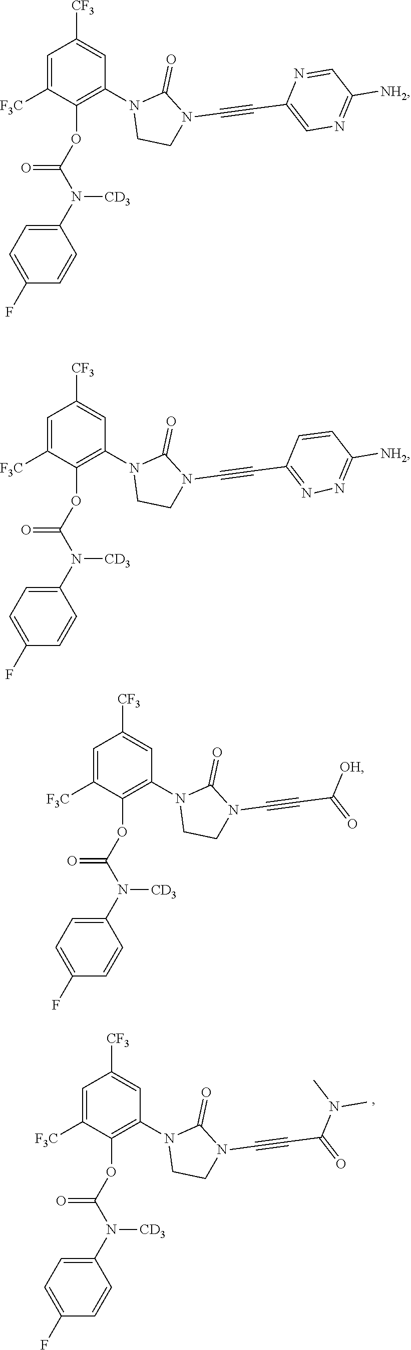 [SMILES: [2H]C([2H])([2H])N(C(=O)Oc1c(N2CCN(C#Cc3cnc(N)cn3)C2=O)cc(C(F)(F)F)cc1C(F)(F)F)c1ccc(F)cc1 ; [2H]C([2H])([2H])N(C(=O)Oc1c(N2CCN(C#Cc3ccc(N)nn3)C2=O)cc(C(F)(F)F)cc1C(F)(F)F)c1ccc(F)cc1 ; [2H]C([2H])([2H])N(C(=O)Oc1c(N2CCN(C#CC(=O)O)C2=O)cc(C(F)(F)F)cc1C(F)(F)F)c1ccc(F)cc1 ; [2H]C([2H])([2H])N(C(=O)Oc1c(N2CCN(C#CC(=O)N(C)C)C2=O)cc(C(F)(F)F)cc1C(F)(F)F)c1ccc(F)cc1]

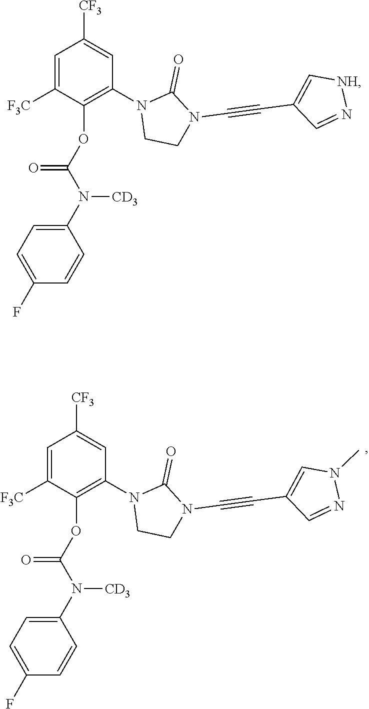 [SMILES: [2H]C([2H])([2H])N(C(=O)Oc1c(N2CCN(C#Cc3cn[nH]c3)C2=O)cc(C(F)(F)F)cc1C(F)(F)F)c1ccc(F)cc1 ; [2H]C([2H])([2H])N(C(=O)Oc1c(N2CCN(C#Cc3cnn(C)c3)C2=O)cc(C(F)(F)F)cc1C(F)(F)F)c1ccc(F)cc1]

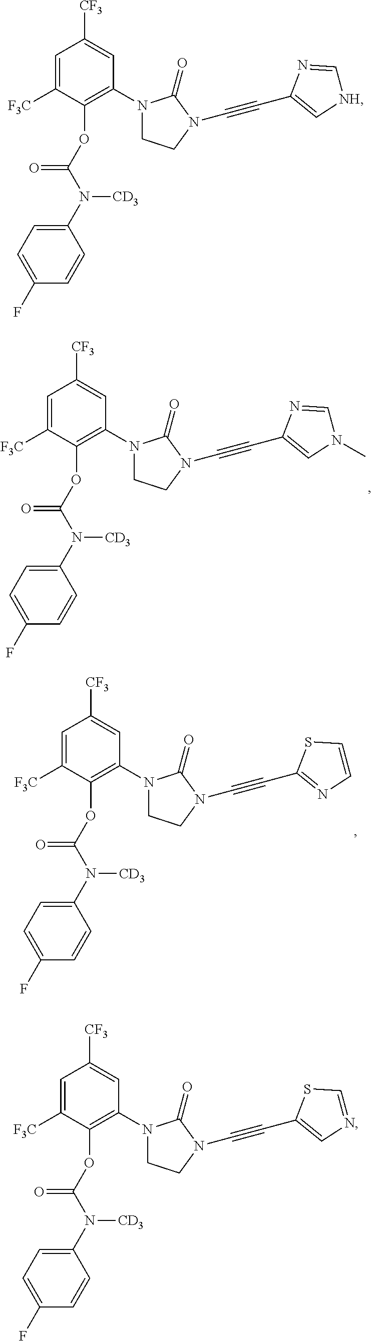 [SMILES: [2H]C([2H])([2H])N(C(=O)Oc1c(N2CCN(C#Cc3c[nH]cn3)C2=O)cc(C(F)(F)F)cc1C(F)(F)F)c1ccc(F)cc1 ; [2H]C([2H])([2H])N(C(=O)Oc1c(N2CCN(C#Cc3cn(C)cn3)C2=O)cc(C(F)(F)F)cc1C(F)(F)F)c1ccc(F)cc1 ; [2H]C([2H])([2H])N(C(=O)Oc1c(N2CCN(C#Cc3nccs3)C2=O)cc(C(F)(F)F)cc1C(F)(F)F)c1ccc(F)cc1 ; [2H]C([2H])([2H])N(C(=O)Oc1c(N2CCN(C#Cc3cncs3)C2=O)cc(C(F)(F)F)cc1C(F)(F)F)c1ccc(F)cc1]

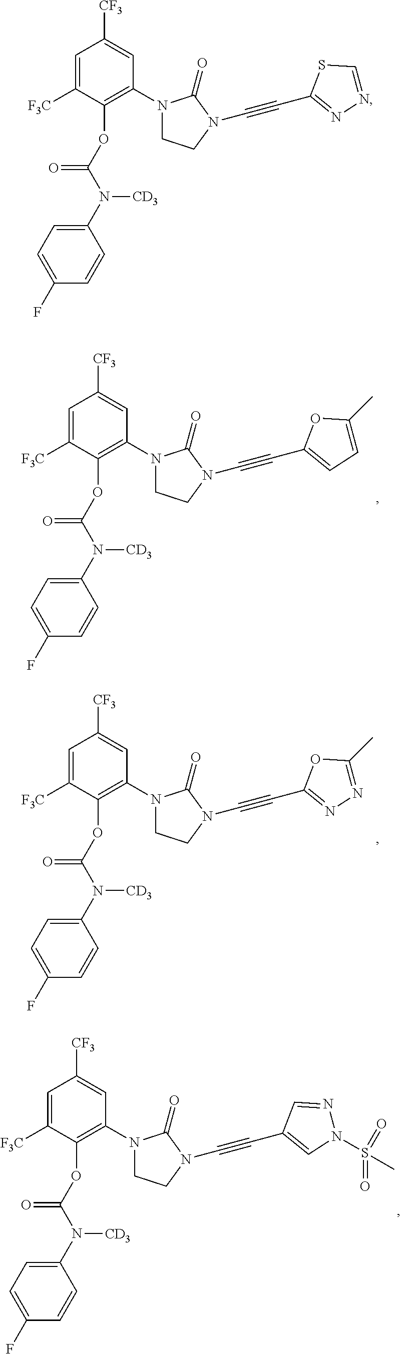 [SMILES: [2H]C([2H])([2H])N(C(=O)Oc1c(N2CCN(C#Cc3nncs3)C2=O)cc(C(F)(F)F)cc1C(F)(F)F)c1ccc(F)cc1 ; [2H]C([2H])([2H])N(C(=O)Oc1c(N2CCN(C#Cc3ccc(C)o3)C2=O)cc(C(F)(F)F)cc1C(F)(F)F)c1ccc(F)cc1 ; [2H]C([2H])([2H])N(C(=O)Oc1c(N2CCN(C#Cc3nnc(C)o3)C2=O)cc(C(F)(F)F)cc1C(F)(F)F)c1ccc(F)cc1 ; [2H]C([2H])([2H])N(C(=O)Oc1c(N2CCN(C#Cc3cnn(S(C)(=O)=O)c3)C2=O)cc(C(F)(F)F)cc1C(F)(F)F)c1ccc(F)cc1]

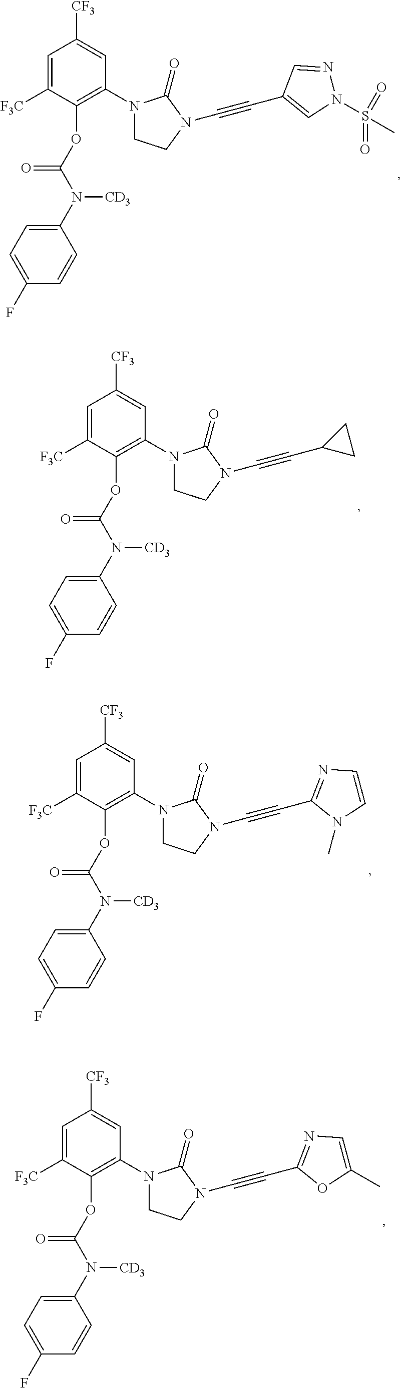 [SMILES: [2H]C([2H])([2H])N(C(=O)Oc1c(N2CCN(C#Cc3cnn(S(C)(=O)=O)c3)C2=O)cc(C(F)(F)F)cc1C(F)(F)F)c1ccc(F)cc1 ; [2H]C([2H])([2H])N(C(=O)Oc1c(N2CCN(C#CC3CC3)C2=O)cc(C(F)(F)F)cc1C(F)(F)F)c1ccc(F)cc1 ; [2H]C([2H])([2H])N(C(=O)Oc1c(N2CCN(C#Cc3nccn3C)C2=O)cc(C(F)(F)F)cc1C(F)(F)F)c1ccc(F)cc1 ; [2H]C([2H])([2H])N(C(=O)Oc1c(N2CCN(C#Cc3ncc(C)o3)C2=O)cc(C(F)(F)F)cc1C(F)(F)F)c1ccc(F)cc1]

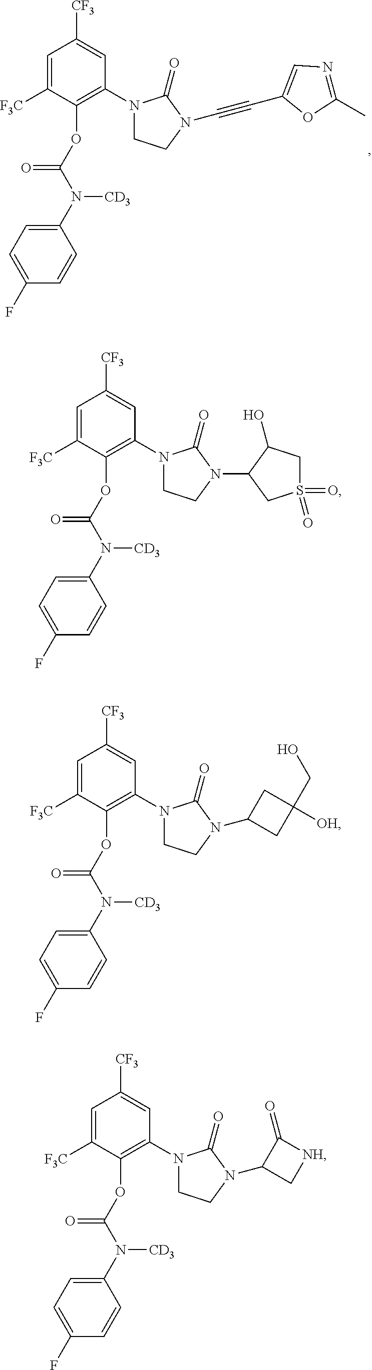 [SMILES: [2H]C([2H])([2H])N(C(=O)Oc1c(N2CCN(C#Cc3cnc(C)o3)C2=O)cc(C(F)(F)F)cc1C(F)(F)F)c1ccc(F)cc1 ; [2H]C([2H])([2H])N(C(=O)Oc1c(N2CCN(C3CS(=O)(=O)CC3O)C2=O)cc(C(F)(F)F)cc1C(F)(F)F)c1ccc(F)cc1 ; [2H]C([2H])([2H])N(C(=O)Oc1c(N2CCN(C3CC(O)(CO)C3)C2=O)cc(C(F)(F)F)cc1C(F)(F)F)c1ccc(F)cc1 ; [2H]C([2H])([2H])N(C(=O)Oc1c(N2CCN(C3CNC3=O)C2=O)cc(C(F)(F)F)cc1C(F)(F)F)c1ccc(F)cc1]

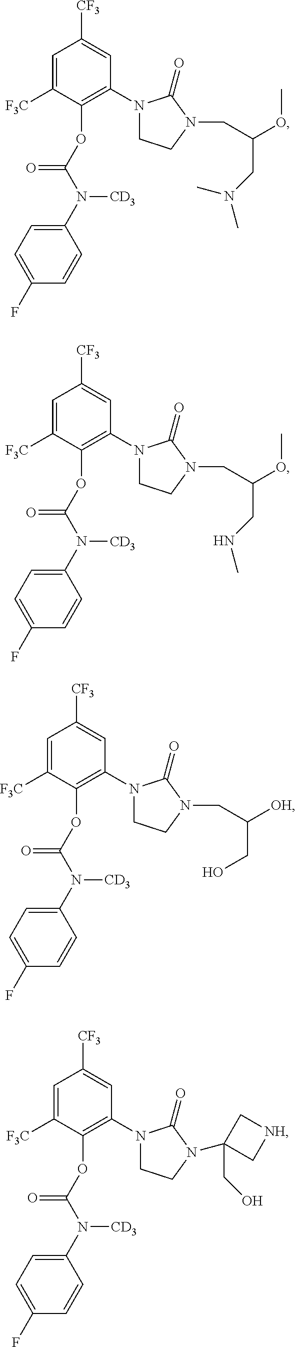 [SMILES: [2H]C([2H])([2H])N(C(=O)Oc1c(N2CCN(CC(CN(C)C)OC)C2=O)cc(C(F)(F)F)cc1C(F)(F)F)c1ccc(F)cc1 ; [2H]C([2H])([2H])N(C(=O)Oc1c(N2CCN(CC(CNC)OC)C2=O)cc(C(F)(F)F)cc1C(F)(F)F)c1ccc(F)cc1 ; [2H]C([2H])([2H])N(C(=O)Oc1c(N2CCN(CC(O)CO)C2=O)cc(C(F)(F)F)cc1C(F)(F)F)c1ccc(F)cc1 ; [2H]C([2H])([2H])N(C(=O)Oc1c(N2CCN(C3(CO)CNC3)C2=O)cc(C(F)(F)F)cc1C(F)(F)F)c1ccc(F)cc1]

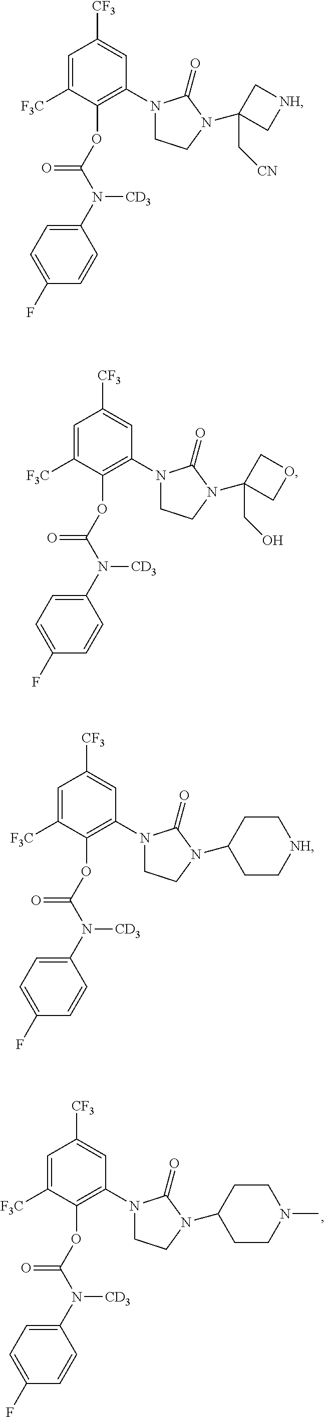 [SMILES: [2H]C([2H])([2H])N(C(=O)Oc1c(N2CCN(C3(CC#N)CNC3)C2=O)cc(C(F)(F)F)cc1C(F)(F)F)c1ccc(F)cc1 ; [2H]C([2H])([2H])N(C(=O)Oc1c(N2CCN(C3(CO)COC3)C2=O)cc(C(F)(F)F)cc1C(F)(F)F)c1ccc(F)cc1 ; [2H]C([2H])([2H])N(C(=O)Oc1c(N2CCN(C3CCNCC3)C2=O)cc(C(F)(F)F)cc1C(F)(F)F)c1ccc(F)cc1 ; [2H]C([2H])([2H])N(C(=O)Oc1c(N2CCN(C3CCN(C)CC3)C2=O)cc(C(F)(F)F)cc1C(F)(F)F)c1ccc(F)cc1]

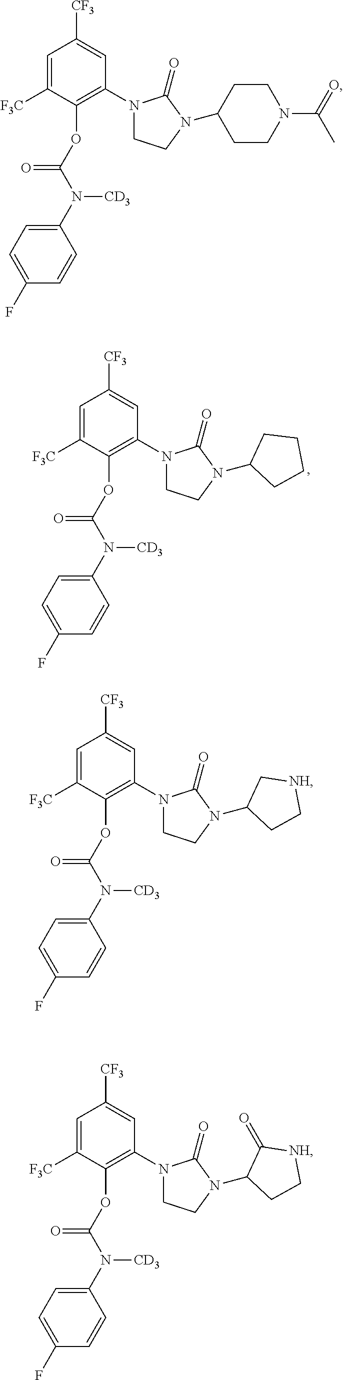 [SMILES: [2H]C([2H])([2H])N(C(=O)Oc1c(N2CCN(C3CCN(C(C)=O)CC3)C2=O)cc(C(F)(F)F)cc1C(F)(F)F)c1ccc(F)cc1 ; [2H]C([2H])([2H])N(C(=O)Oc1c(N2CCN(C3CCCC3)C2=O)cc(C(F)(F)F)cc1C(F)(F)F)c1ccc(F)cc1 ; [2H]C([2H])([2H])N(C(=O)Oc1c(N2CCN(C3CCNC3)C2=O)cc(C(F)(F)F)cc1C(F)(F)F)c1ccc(F)cc1 ; [2H]C([2H])([2H])N(C(=O)Oc1c(N2CCN(C3CCNC3=O)C2=O)cc(C(F)(F)F)cc1C(F)(F)F)c1ccc(F)cc1]

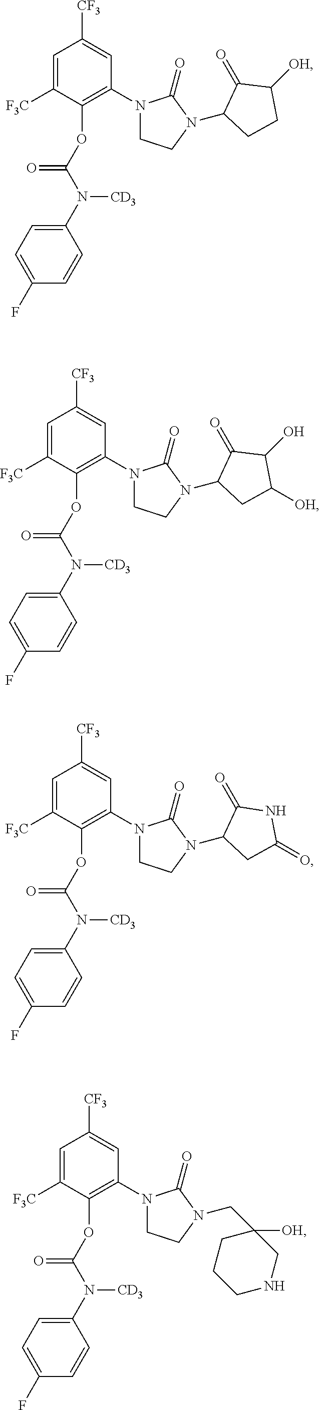 [SMILES: [2H]C([2H])([2H])N(C(=O)Oc1c(N2CCN(C3CCC(O)C3=O)C2=O)cc(C(F)(F)F)cc1C(F)(F)F)c1ccc(F)cc1 ; [2H]C([2H])([2H])N(C(=O)Oc1c(N2CCN(C3CC(O)C(O)C3=O)C2=O)cc(C(F)(F)F)cc1C(F)(F)F)c1ccc(F)cc1 ; [2H]C([2H])([2H])N(C(=O)Oc1c(N2CCN(C3CC(=O)NC3=O)C2=O)cc(C(F)(F)F)cc1C(F)(F)F)c1ccc(F)cc1 ; [2H]C([2H])([2H])N(C(=O)Oc1c(N2CCN(CC3(O)CCCNC3)C2=O)cc(C(F)(F)F)cc1C(F)(F)F)c1ccc(F)cc1]

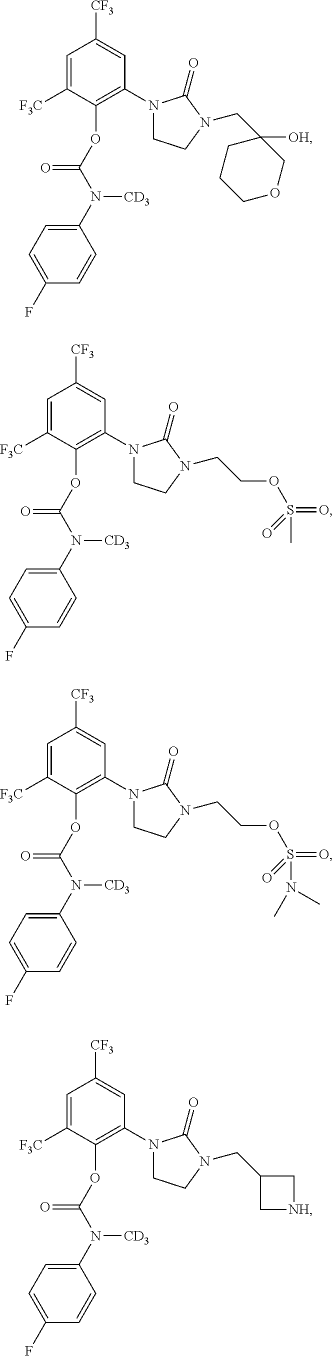 [SMILES: [2H]C([2H])([2H])N(C(=O)Oc1c(N2CCN(CC3(O)CCCOC3)C2=O)cc(C(F)(F)F)cc1C(F)(F)F)c1ccc(F)cc1 ; [2H]C([2H])([2H])N(C(=O)Oc1c(N2CCN(CCOS(C)(=O)=O)C2=O)cc(C(F)(F)F)cc1C(F)(F)F)c1ccc(F)cc1 ; [2H]C([2H])([2H])N(C(=O)Oc1c(N2CCN(CCOS(=O)(=O)N(C)C)C2=O)cc(C(F)(F)F)cc1C(F)(F)F)c1ccc(F)cc1 ; [2H]C([2H])([2H])N(C(=O)Oc1c(N2CCN(CC3CNC3)C2=O)cc(C(F)(F)F)cc1C(F)(F)F)c1ccc(F)cc1]

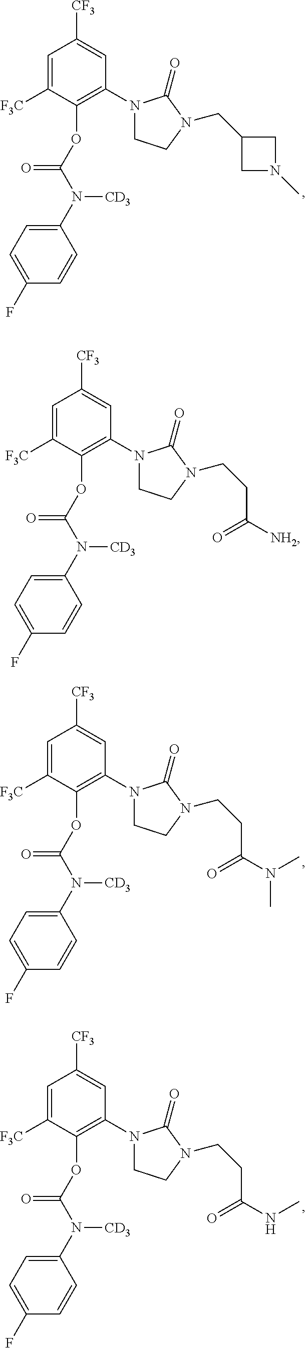 [SMILES: [2H]C([2H])([2H])N(C(=O)Oc1c(N2CCN(CC3CN(C)C3)C2=O)cc(C(F)(F)F)cc1C(F)(F)F)c1ccc(F)cc1 ; [2H]C([2H])([2H])N(C(=O)Oc1c(N2CCN(CCC(N)=O)C2=O)cc(C(F)(F)F)cc1C(F)(F)F)c1ccc(F)cc1 ; [2H]C([2H])([2H])N(C(=O)Oc1c(N2CCN(CCC(=O)N(C)C)C2=O)cc(C(F)(F)F)cc1C(F)(F)F)c1ccc(F)cc1 ; [2H]C([2H])([2H])N(C(=O)Oc1c(N2CCN(CCC(=O)NC)C2=O)cc(C(F)(F)F)cc1C(F)(F)F)c1ccc(F)cc1]

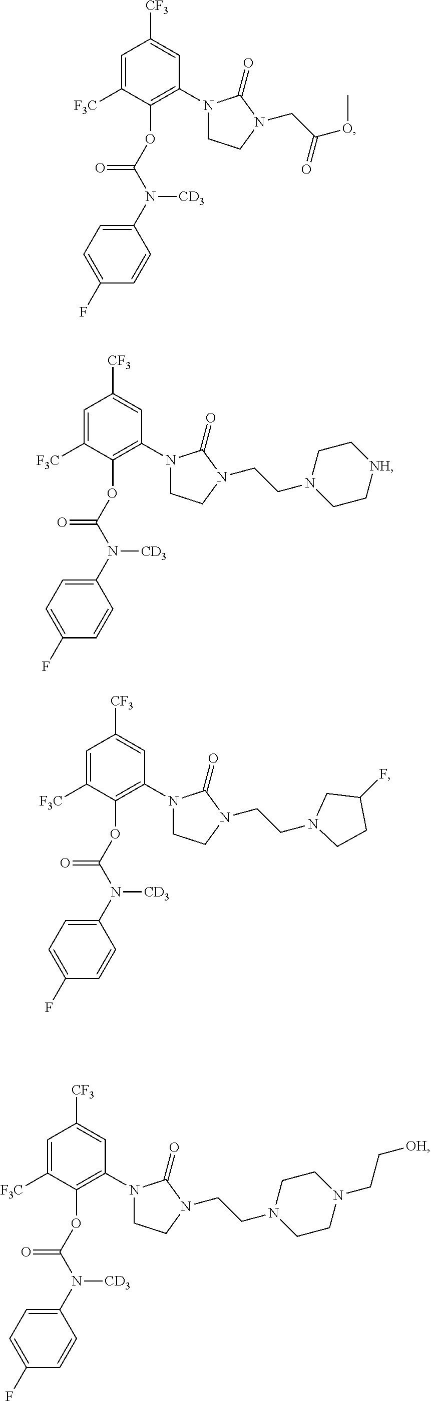 [SMILES: [2H]C([2H])([2H])N(C(=O)Oc1c(N2CCN(CC(=O)OC)C2=O)cc(C(F)(F)F)cc1C(F)(F)F)c1ccc(F)cc1 ; [2H]C([2H])([2H])N(C(=O)Oc1c(N2CCN(CCN3CCNCC3)C2=O)cc(C(F)(F)F)cc1C(F)(F)F)c1ccc(F)cc1 ; [2H]C([2H])([2H])N(C(=O)Oc1c(N2CCN(CCN3CCC(F)C3)C2=O)cc(C(F)(F)F)cc1C(F)(F)F)c1ccc(F)cc1 ; [2H]C([2H])([2H])N(C(=O)Oc1c(N2CCN(CCN3CCN(CCO)CC3)C2=O)cc(C(F)(F)F)cc1C(F)(F)F)c1ccc(F)cc1]

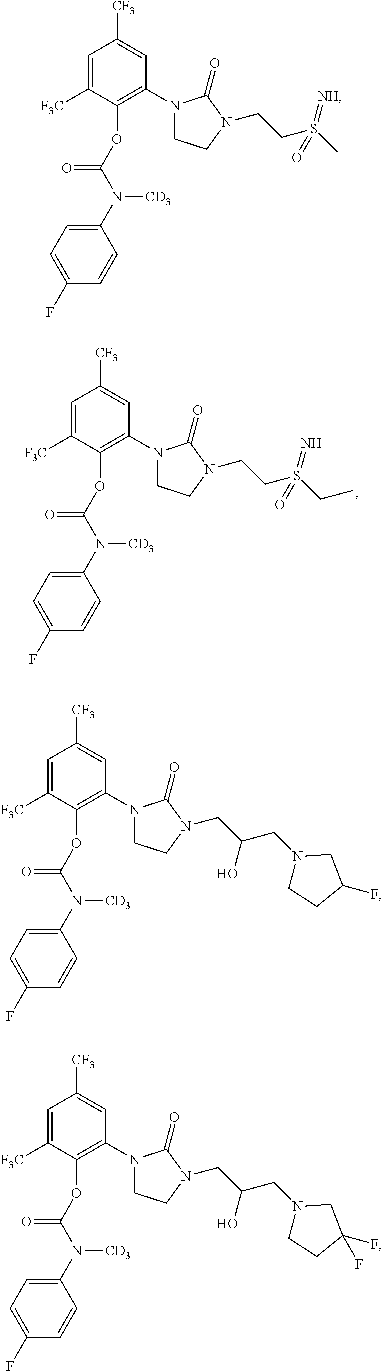 [SMILES: [2H]C([2H])([2H])N(C(=O)Oc1c(N2CCN(CCS(C)(=N)=O)C2=O)cc(C(F)(F)F)cc1C(F)(F)F)c1ccc(F)cc1 ; [2H]C([2H])([2H])N(C(=O)Oc1c(N2CCN(CCS(=N)(=O)CC)C2=O)cc(C(F)(F)F)cc1C(F)(F)F)c1ccc(F)cc1 ; [2H]C([2H])([2H])N(C(=O)Oc1c(N2CCN(CC(O)CN3CCC(F)C3)C2=O)cc(C(F)(F)F)cc1C(F)(F)F)c1ccc(F)cc1 ; [2H]C([2H])([2H])N(C(=O)Oc1c(N2CCN(CC(O)CN3CCC(F)(F)C3)C2=O)cc(C(F)(F)F)cc1C(F)(F)F)c1ccc(F)cc1]

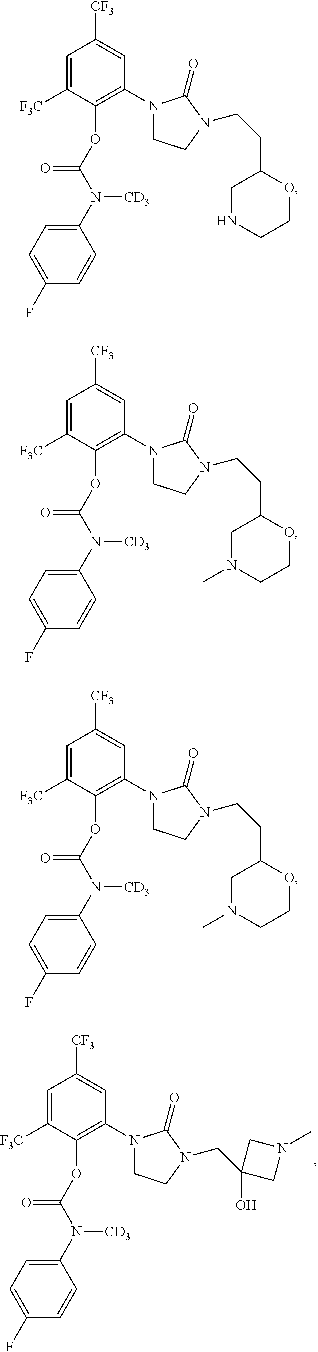 [SMILES: [2H]C([2H])([2H])N(C(=O)Oc1c(N2CCN(CCC3CNCCO3)C2=O)cc(C(F)(F)F)cc1C(F)(F)F)c1ccc(F)cc1 ; [2H]C([2H])([2H])N(C(=O)Oc1c(N2CCN(CCC3CN(C)CCO3)C2=O)cc(C(F)(F)F)cc1C(F)(F)F)c1ccc(F)cc1 ; [2H]C([2H])([2H])N(C(=O)Oc1c(N2CCN(CCC3CN(C)CCO3)C2=O)cc(C(F)(F)F)cc1C(F)(F)F)c1ccc(F)cc1 ; [2H]C([2H])([2H])N(C(=O)Oc1c(N2CCN(CC3(O)CN(C)C3)C2=O)cc(C(F)(F)F)cc1C(F)(F)F)c1ccc(F)cc1]

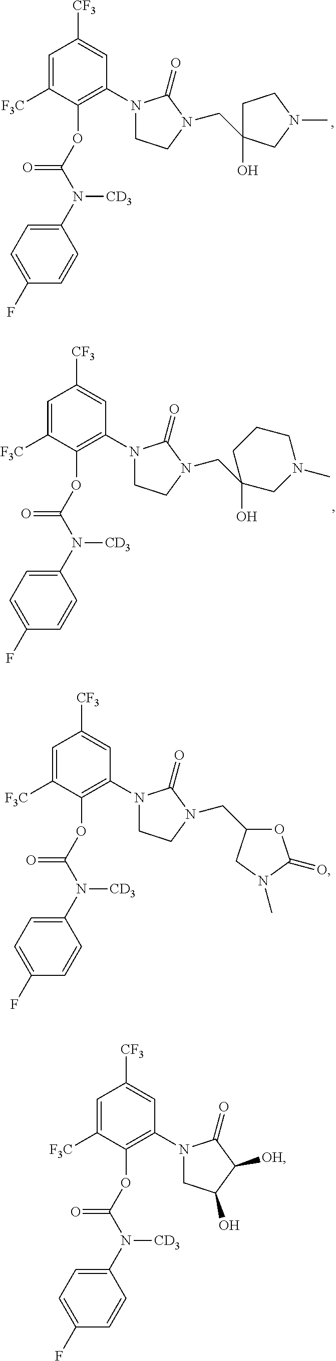 [SMILES: [2H]C([2H])([2H])N(C(=O)Oc1c(N2CCN(CC3(O)CCN(C)C3)C2=O)cc(C(F)(F)F)cc1C(F)(F)F)c1ccc(F)cc1 ; [2H]C([2H])([2H])N(C(=O)Oc1c(N2CCN(CC3(O)CCCN(C)C3)C2=O)cc(C(F)(F)F)cc1C(F)(F)F)c1ccc(F)cc1 ; [2H]C([2H])([2H])N(C(=O)Oc1c(N2CCN(CC3CN(C)C(=O)O3)C2=O)cc(C(F)(F)F)cc1C(F)(F)F)c1ccc(F)cc1 ; [2H]C([2H])([2H])N(C(=O)Oc1c(N2C[C@H](O)[C@H](O)C2=O)cc(C(F)(F)F)cc1C(F)(F)F)c1ccc(F)cc1]

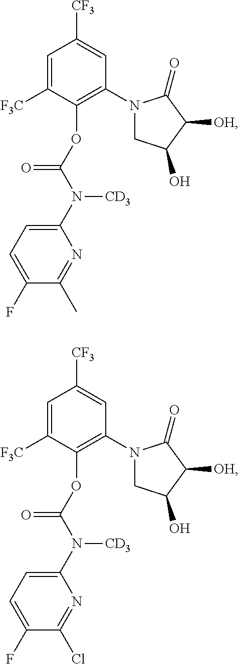

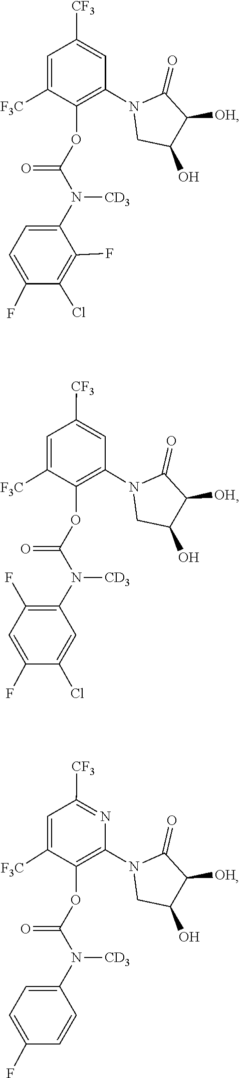 [SMILES: [2H]C([2H])([2H])N(C(=O)Oc1c(N2C[C@H](O)[C@H](O)C2=O)cc(C(F)(F)F)cc1C(F)(F)F)c1ccc(F)c(Cl)c1F ; [2H]C([2H])([2H])N(C(=O)Oc1c(N2C[C@H](O)[C@H](O)C2=O)cc(C(F)(F)F)cc1C(F)(F)F)c1cc(Cl)c(F)cc1F ; [2H]C([2H])([2H])N(C(=O)Oc1c(C(F)(F)F)cc(C(F)(F)F)nc1N1C[C@H](O)[C@H](O)C1=O)c1ccc(F)cc1]

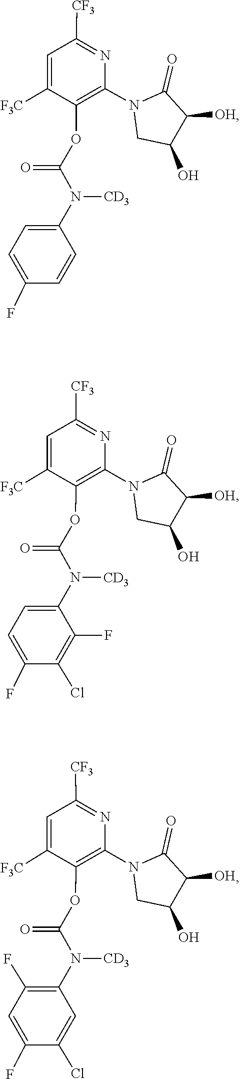 [SMILES: [2H]C([2H])([2H])N(C(=O)Oc1c(C(F)(F)F)cc(C(F)(F)F)nc1N1C[C@H](O)[C@H](O)C1=O)c1ccc(F)cc1 ; [2H]C([2H])([2H])N(C(=O)Oc1c(C(F)(F)F)cc(C(F)(F)F)nc1N1C[C@H](O)[C@H](O)C1=O)c1ccc(F)c(Cl)c1F ; [2H]C([2H])([2H])N(C(=O)Oc1c(C(F)(F)F)cc(C(F)(F)F)nc1N1C[C@H](O)[C@H](O)C1=O)c1cc(Cl)c(F)cc1F]

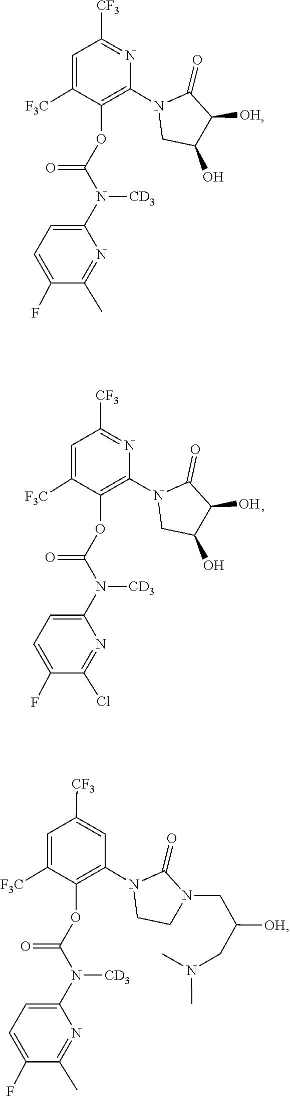 [SMILES: [2H]C([2H])([2H])N(C(=O)Oc1c(C(F)(F)F)cc(C(F)(F)F)nc1N1C[C@H](O)[C@H](O)C1=O)c1ccc(F)c(C)n1 ; [2H]C([2H])([2H])N(C(=O)Oc1c(C(F)(F)F)cc(C(F)(F)F)nc1N1C[C@H](O)[C@H](O)C1=O)c1ccc(F)c(Cl)n1 ; [2H]C([2H])([2H])N(C(=O)Oc1c(N2CCN(CC(O)CN(C)C)C2=O)cc(C(F)(F)F)cc1C(F)(F)F)c1ccc(F)c(C)n1]

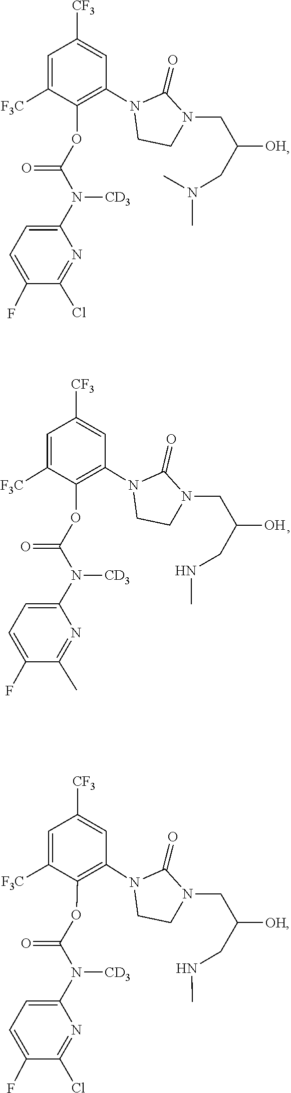 [SMILES: [2H]C([2H])([2H])N(C(=O)Oc1c(N2CCN(CC(O)CN(C)C)C2=O)cc(C(F)(F)F)cc1C(F)(F)F)c1ccc(F)c(Cl)n1 ; [2H]C([2H])([2H])N(C(=O)Oc1c(N2CCN(CC(O)CNC)C2=O)cc(C(F)(F)F)cc1C(F)(F)F)c1ccc(F)c(C)n1 ; [2H]C([2H])([2H])N(C(=O)Oc1c(N2CCN(CC(O)CNC)C2=O)cc(C(F)(F)F)cc1C(F)(F)F)c1ccc(F)c(Cl)n1]

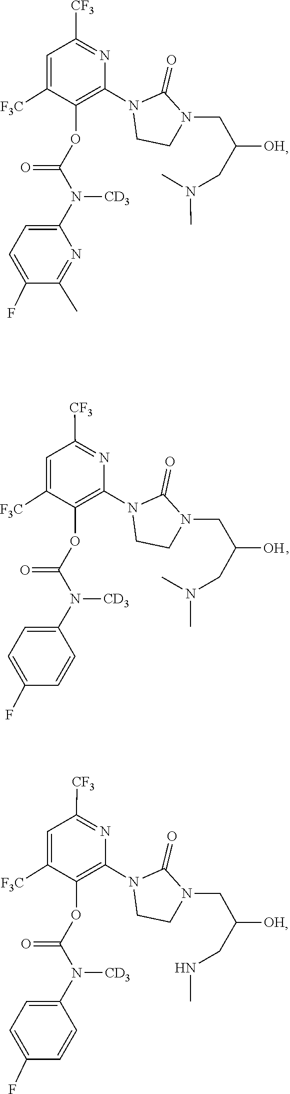 [SMILES: [2H]C([2H])([2H])N(C(=O)Oc1c(C(F)(F)F)cc(C(F)(F)F)nc1N1CCN(CC(O)CN(C)C)C1=O)c1ccc(F)c(C)n1 ; [2H]C([2H])([2H])N(C(=O)Oc1c(C(F)(F)F)cc(C(F)(F)F)nc1N1CCN(CC(O)CN(C)C)C1=O)c1ccc(F)cc1 ; [2H]C([2H])([2H])N(C(=O)Oc1c(C(F)(F)F)cc(C(F)(F)F)nc1N1CCN(CC(O)CNC)C1=O)c1ccc(F)cc1]

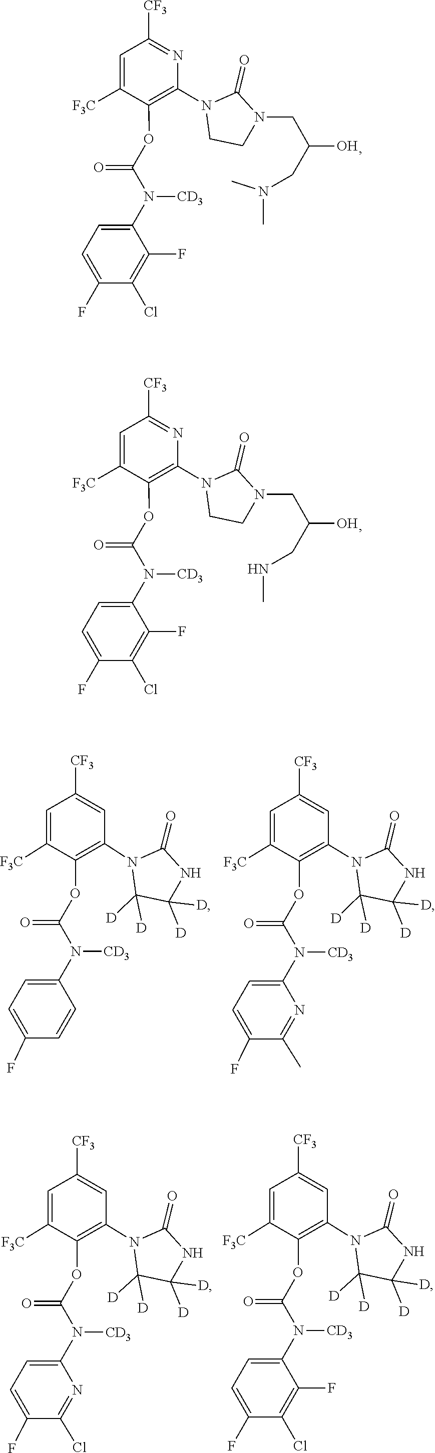

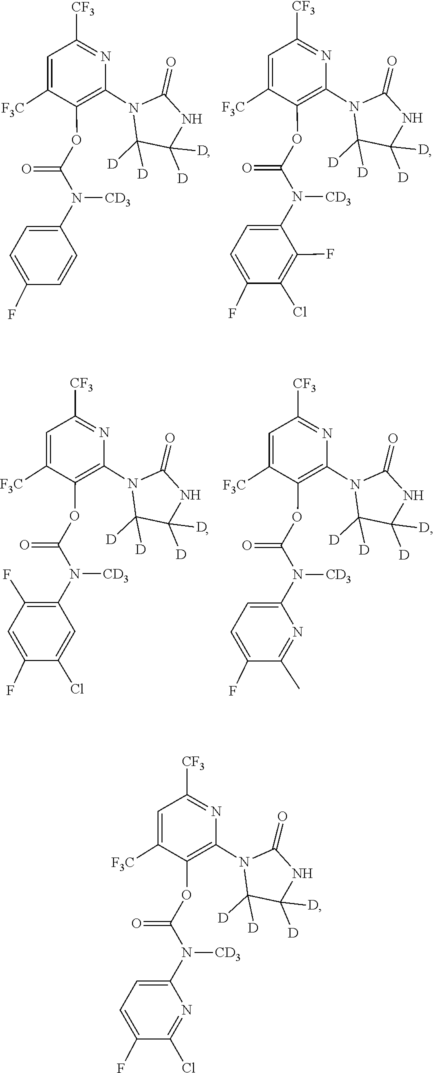

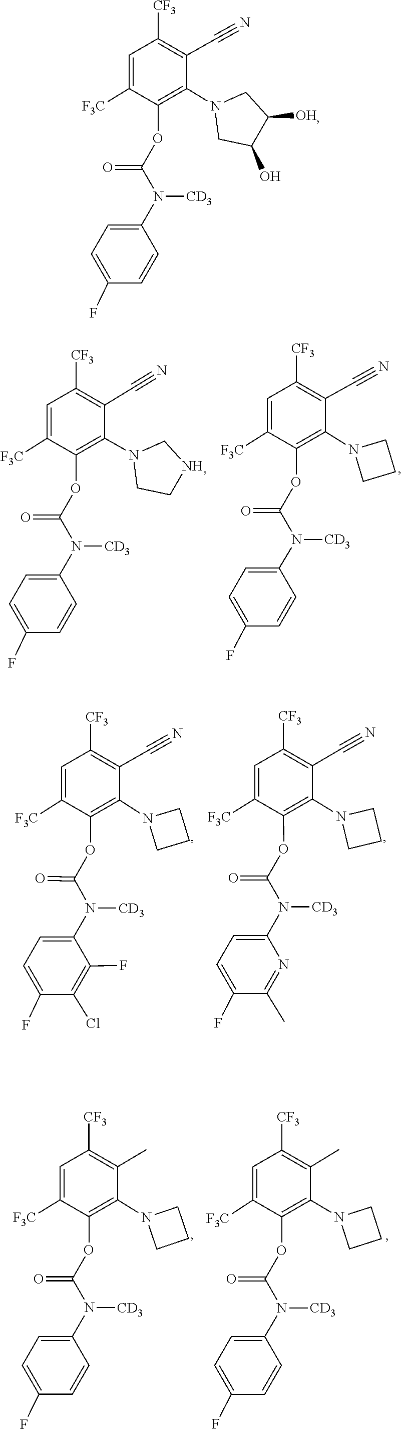

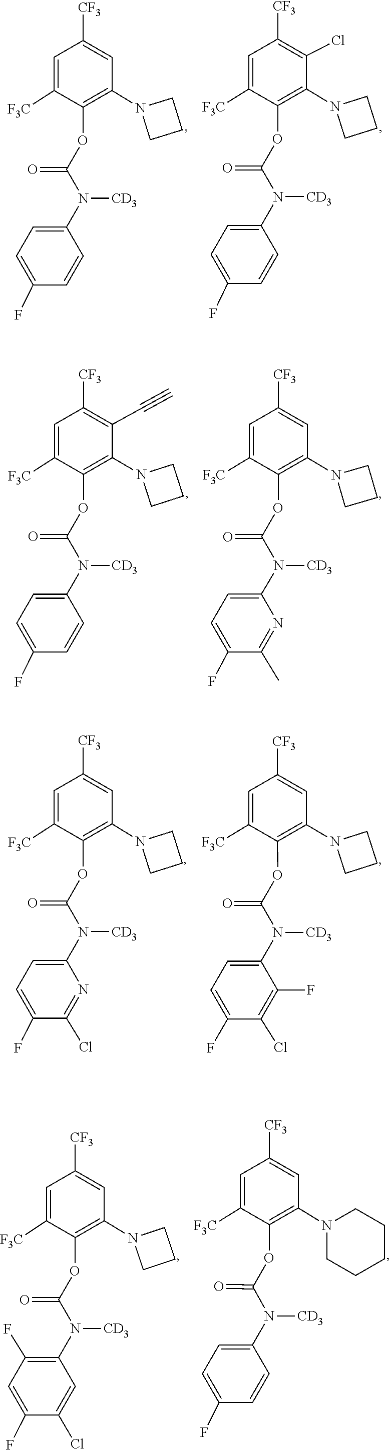

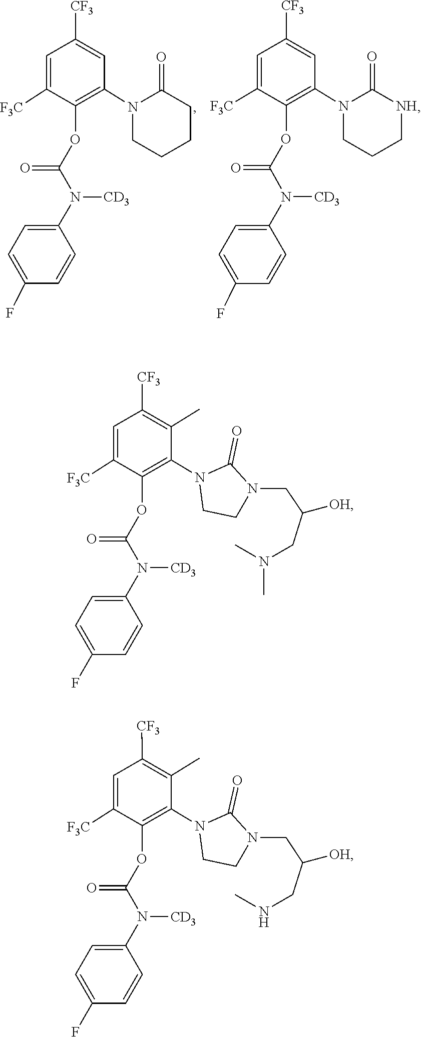

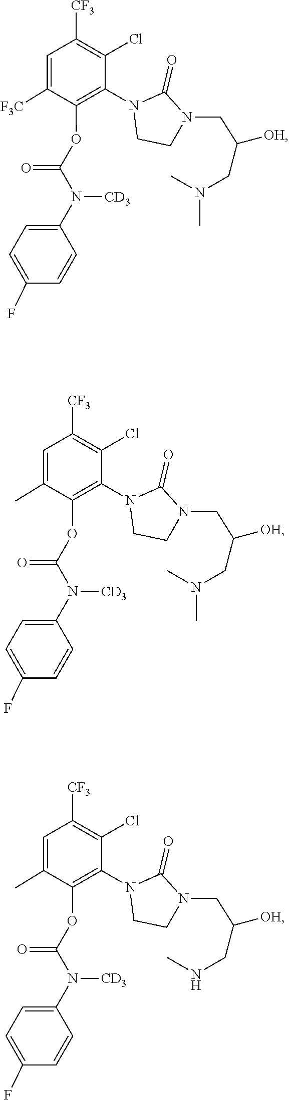 [SMILES: [2H]C([2H])([2H])N(C(=O)Oc1c(C(F)(F)F)cc(C(F)(F)F)c(Cl)c1N1CCN(CC(O)CN(C)C)C1=O)c1ccc(F)cc1 ; [2H]C([2H])([2H])N(C(=O)Oc1c(C)cc(C(F)(F)F)c(Cl)c1N1CCN(CC(O)CN(C)C)C1=O)c1ccc(F)cc1 ; [2H]C([2H])([2H])N(C(=O)Oc1c(C)cc(C(F)(F)F)c(Cl)c1N1CCN(CC(O)CNC)C1=O)c1ccc(F)cc1]

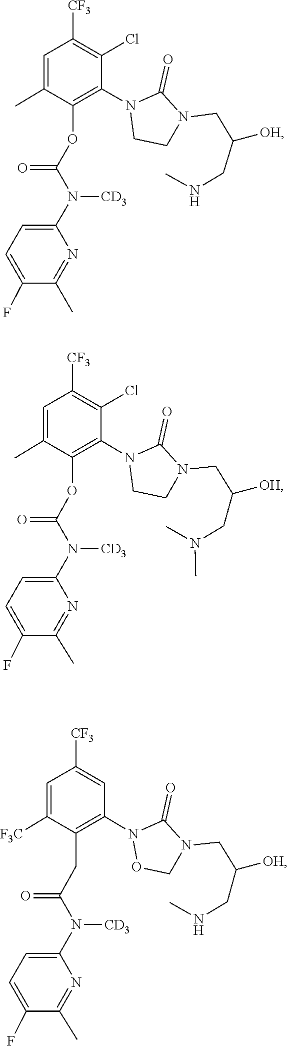

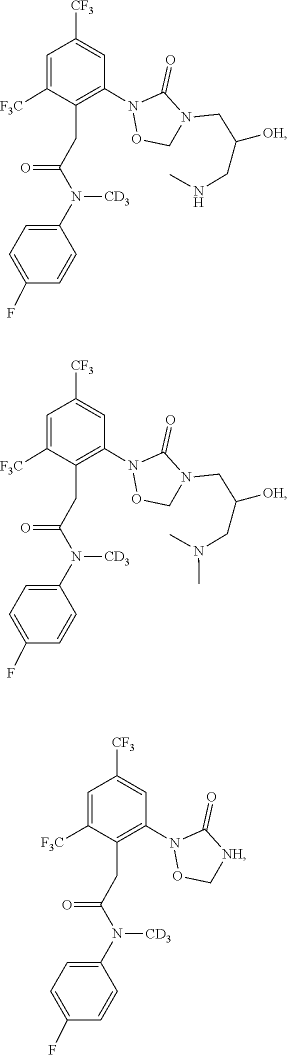 [SMILES: [2H]C([2H])([2H])N(C(=O)Cc1c(N2OCN(CC(O)CNC)C2=O)cc(C(F)(F)F)cc1C(F)(F)F)c1ccc(F)cc1 ; [2H]C([2H])([2H])N(C(=O)Cc1c(N2OCN(CC(O)CN(C)C)C2=O)cc(C(F)(F)F)cc1C(F)(F)F)c1ccc(F)cc1 ; [2H]C([2H])([2H])N(C(=O)Cc1c(N2OCNC2=O)cc(C(F)(F)F)cc1C(F)(F)F)c1ccc(F)cc1]

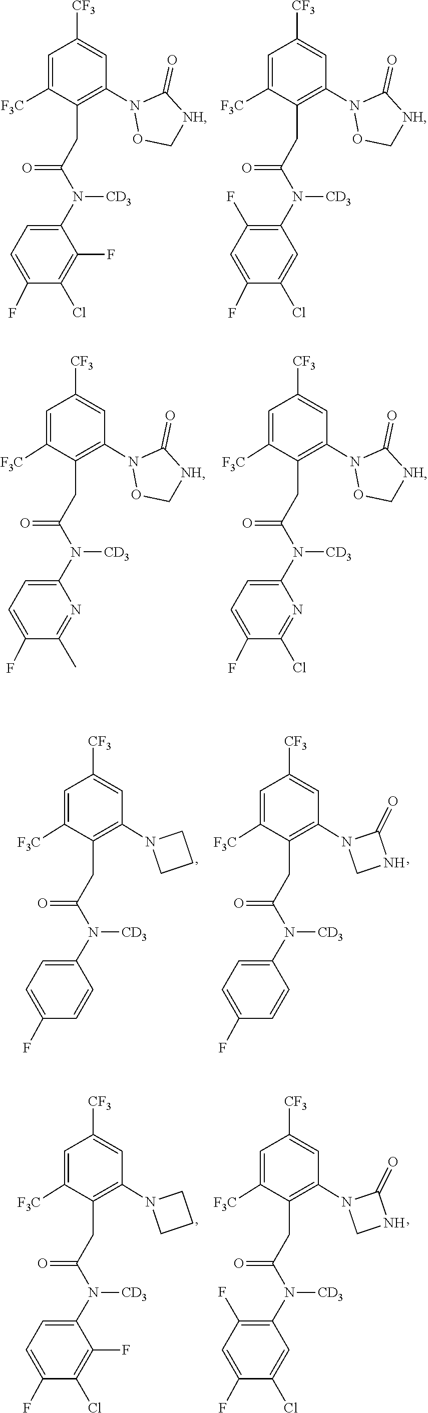

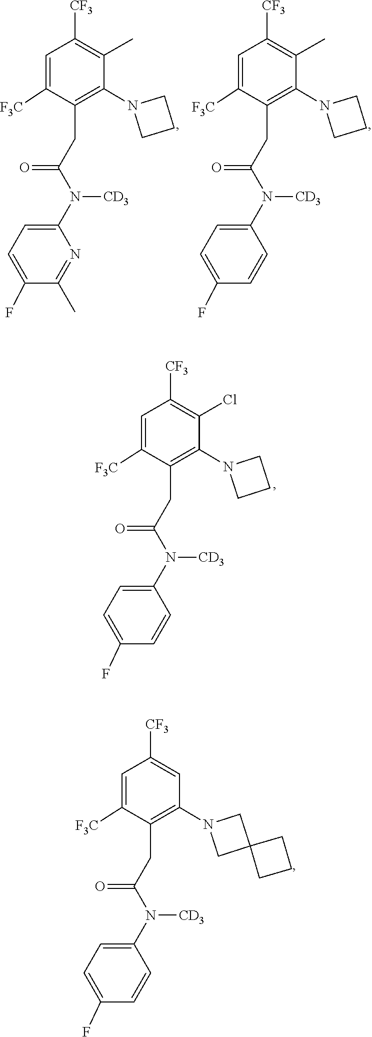

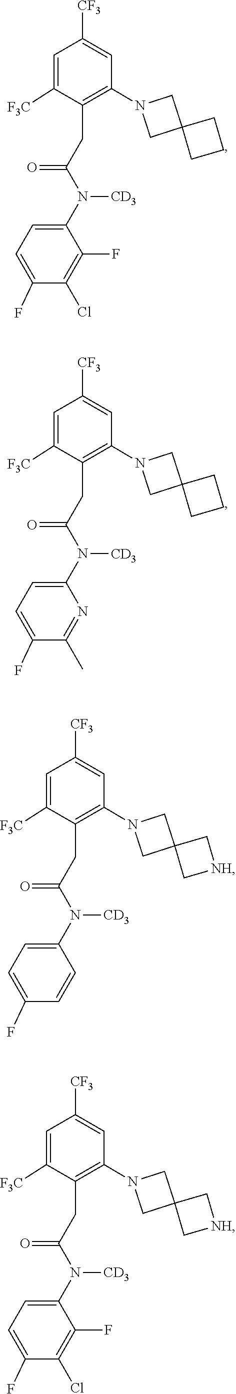 [SMILES: [2H]C([2H])([2H])N(C(=O)Cc1c(N2CC3(CCC3)C2)cc(C(F)(F)F)cc1C(F)(F)F)c1ccc(F)c(Cl)c1F ; [2H]C([2H])([2H])N(C(=O)Cc1c(N2CC3(CCC3)C2)cc(C(F)(F)F)cc1C(F)(F)F)c1ccc(F)c(C)n1 ; [2H]C([2H])([2H])N(C(=O)Cc1c(N2CC3(CNC3)C2)cc(C(F)(F)F)cc1C(F)(F)F)c1ccc(F)cc1 ; [2H]C([2H])([2H])N(C(=O)Cc1c(N2CC3(CNC3)C2)cc(C(F)(F)F)cc1C(F)(F)F)c1ccc(F)c(Cl)c1F]

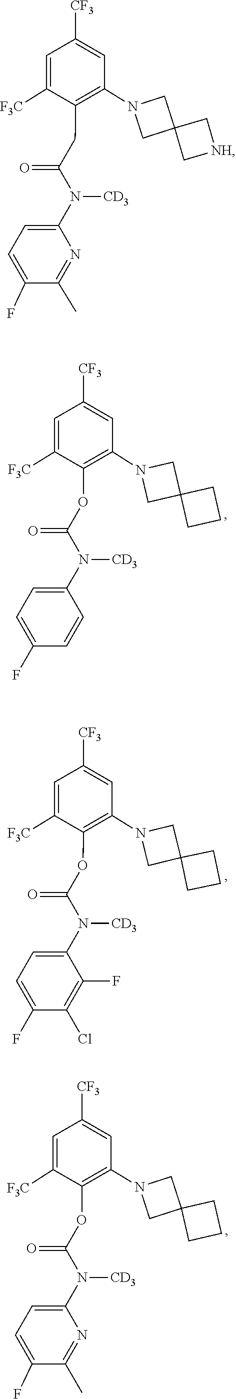 [SMILES: [2H]C([2H])([2H])N(C(=O)Cc1c(N2CC3(CNC3)C2)cc(C(F)(F)F)cc1C(F)(F)F)c1ccc(F)c(C)n1 ; [2H]C([2H])([2H])N(C(=O)Oc1c(N2CC3(CCC3)C2)cc(C(F)(F)F)cc1C(F)(F)F)c1ccc(F)cc1 ; [2H]C([2H])([2H])N(C(=O)Oc1c(N2CC3(CCC3)C2)cc(C(F)(F)F)cc1C(F)(F)F)c1ccc(F)c(Cl)c1F ; [2H]C([2H])([2H])N(C(=O)Oc1c(N2CC3(CCC3)C2)cc(C(F)(F)F)cc1C(F)(F)F)c1ccc(F)c(C)n1]

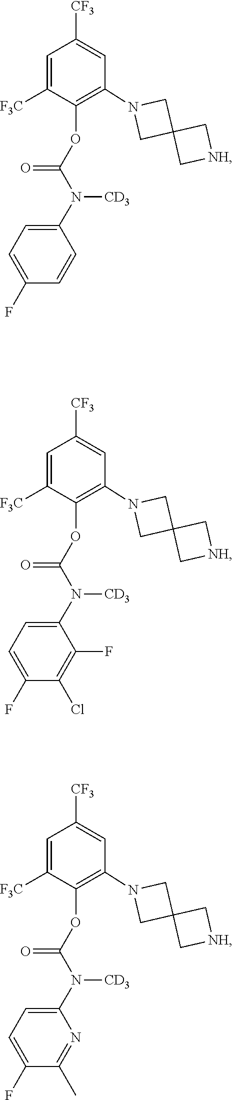 [SMILES: [2H]C([2H])([2H])N(C(=O)Oc1c(N2CC3(CNC3)C2)cc(C(F)(F)F)cc1C(F)(F)F)c1ccc(F)cc1 ; [2H]C([2H])([2H])N(C(=O)Oc1c(N2CC3(CNC3)C2)cc(C(F)(F)F)cc1C(F)(F)F)c1ccc(F)c(Cl)c1F ; [2H]C([2H])([2H])N(C(=O)Oc1c(N2CC3(CNC3)C2)cc(C(F)(F)F)cc1C(F)(F)F)c1ccc(F)c(C)n1]

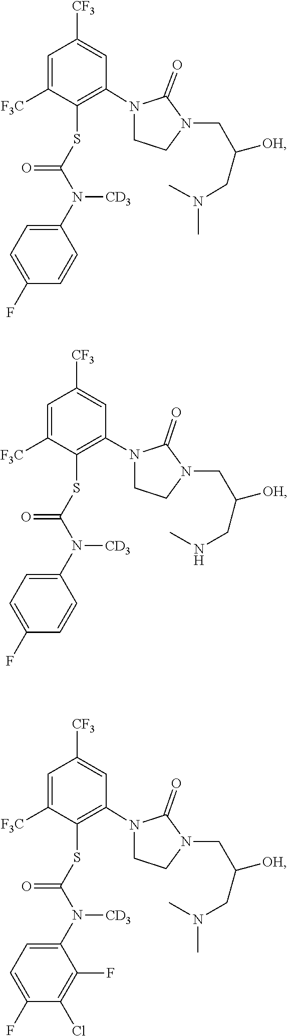 [SMILES: [2H]C([2H])([2H])N(C(=O)Sc1c(N2CCN(CC(O)CN(C)C)C2=O)cc(C(F)(F)F)cc1C(F)(F)F)c1ccc(F)cc1 ; [2H]C([2H])([2H])N(C(=O)Sc1c(N2CCN(CC(O)CNC)C2=O)cc(C(F)(F)F)cc1C(F)(F)F)c1ccc(F)cc1 ; [2H]C([2H])([2H])N(C(=O)Sc1c(N2CCN(CC(O)CN(C)C)C2=O)cc(C(F)(F)F)cc1C(F)(F)F)c1ccc(F)c(Cl)c1F]

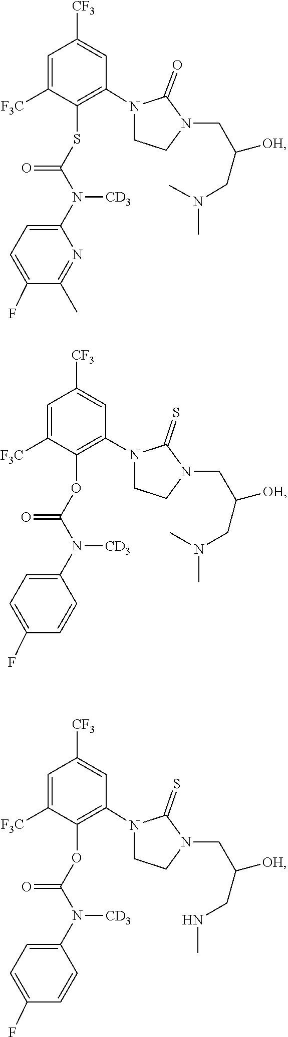

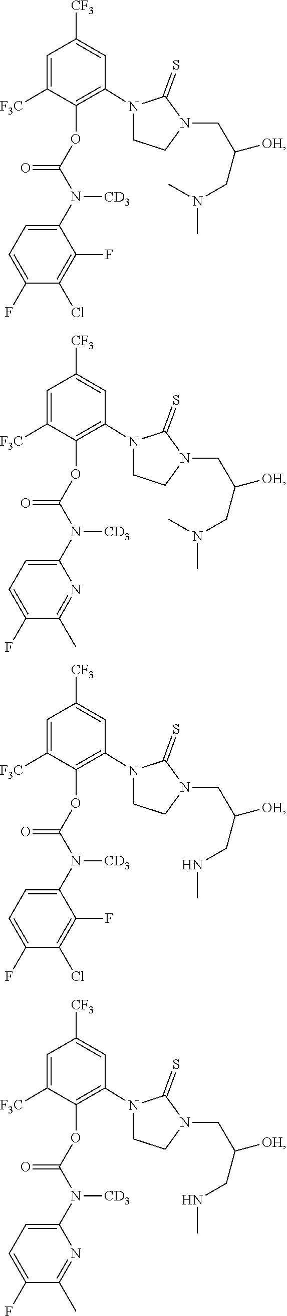

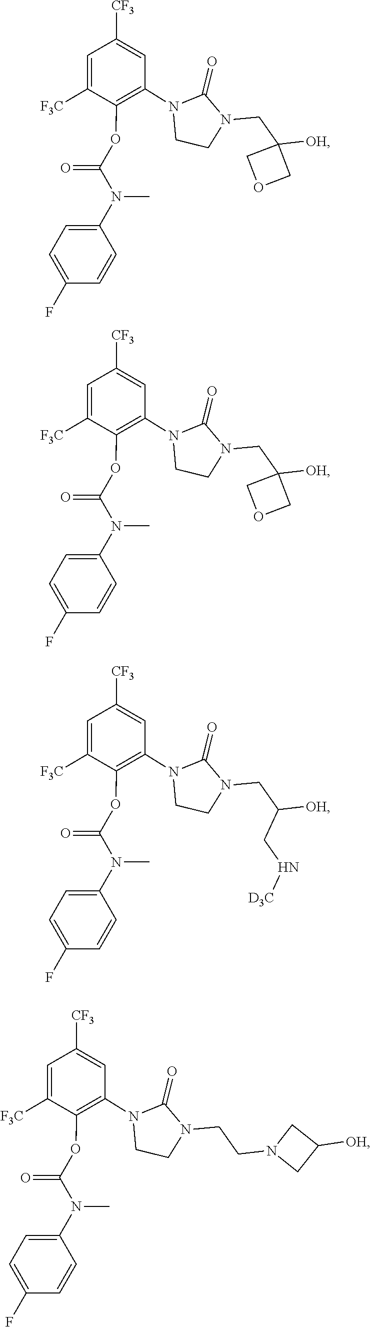

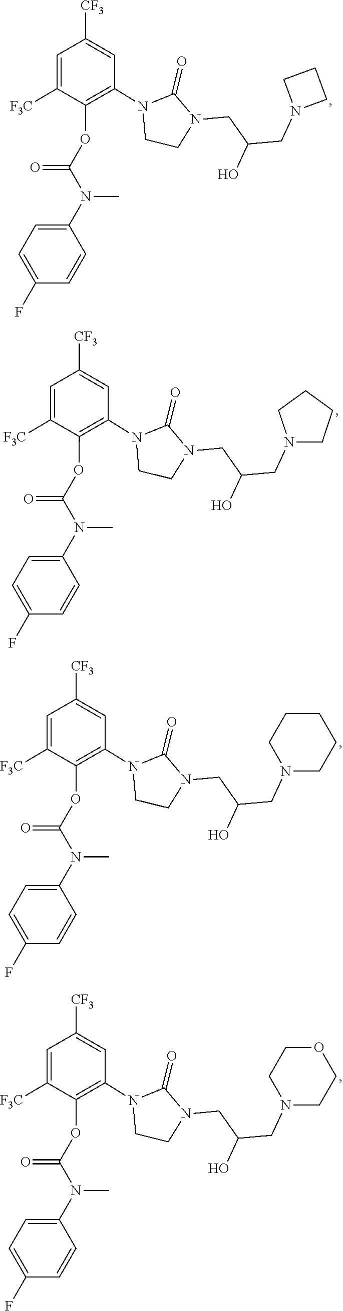 [SMILES: CN(C(=O)Oc1c(N2CCN(CC(O)CN3CCC3)C2=O)cc(C(F)(F)F)cc1C(F)(F)F)c1ccc(F)cc1 ; CN(C(=O)Oc1c(N2CCN(CC(O)CN3CCCC3)C2=O)cc(C(F)(F)F)cc1C(F)(F)F)c1ccc(F)cc1 ; CN(C(=O)Oc1c(N2CCN(CC(O)CN3CCCCC3)C2=O)cc(C(F)(F)F)cc1C(F)(F)F)c1ccc(F)cc1 ; CN(C(=O)Oc1c(N2CCN(CC(O)CN3CCOCC3)C2=O)cc(C(F)(F)F)cc1C(F)(F)F)c1ccc(F)cc1]

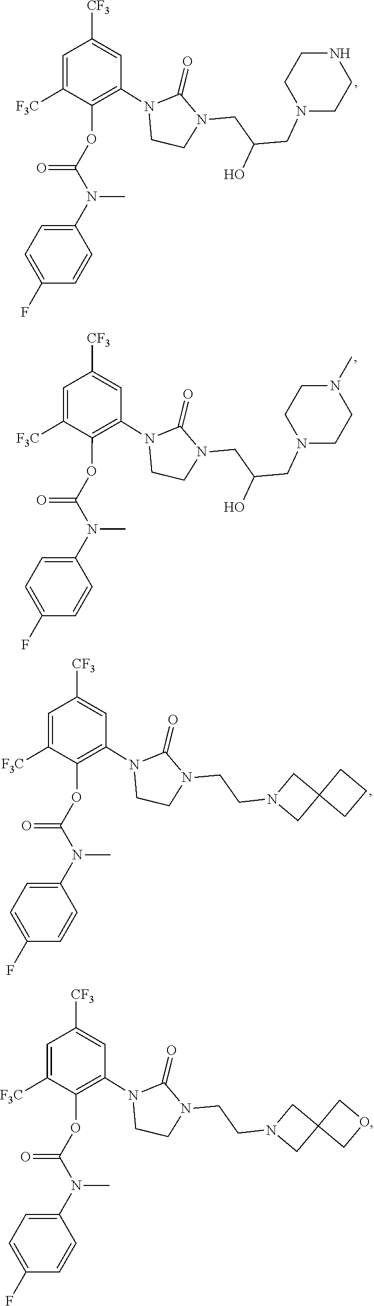 [SMILES: CN(C(=O)Oc1c(N2CCN(CC(O)CN3CCNCC3)C2=O)cc(C(F)(F)F)cc1C(F)(F)F)c1ccc(F)cc1 ; CN1CCN(CC(O)CN2CCN(c3cc(C(F)(F)F)cc(C(F)(F)F)c3OC(=O)N(C)c3ccc(F)cc3)C2=O)CC1 ; CN(C(=O)Oc1c(N2CCN(CCN3CC4(CCC4)C3)C2=O)cc(C(F)(F)F)cc1C(F)(F)F)c1ccc(F)cc1 ; CN(C(=O)Oc1c(N2CCN(CCN3CC4(COC4)C3)C2=O)cc(C(F)(F)F)cc1C(F)(F)F)c1ccc(F)cc1]

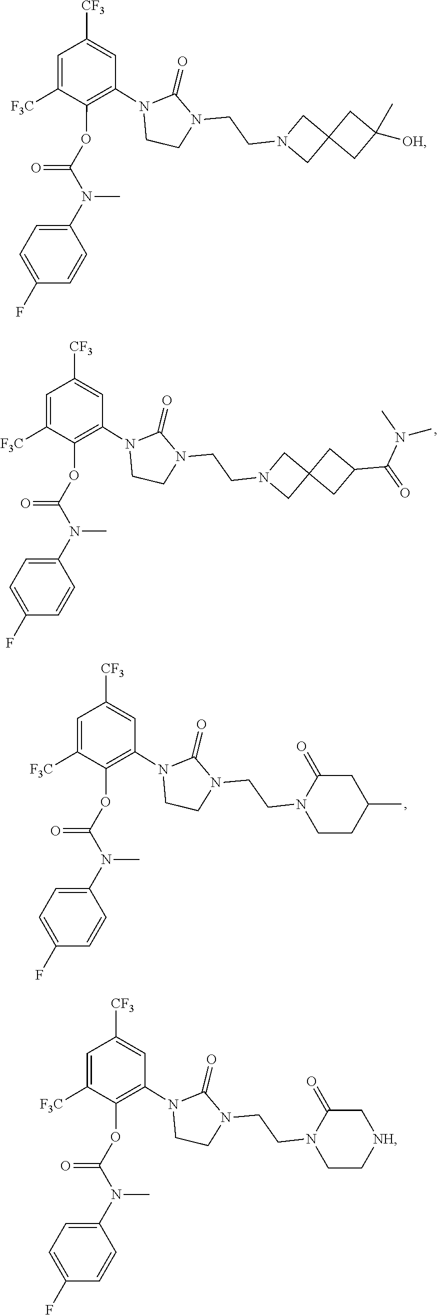 [SMILES: CN(C(=O)Oc1c(N2CCN(CCN3CC4(C3)CC(C)(O)C4)C2=O)cc(C(F)(F)F)cc1C(F)(F)F)c1ccc(F)cc1 ; CN(C)C(=O)C1CC2(C1)CN(CCN1CCN(c3cc(C(F)(F)F)cc(C(F)(F)F)c3OC(=O)N(C)c3ccc(F)cc3)C1=O)C2 ; CC1CCN(CCN2CCN(c3cc(C(F)(F)F)cc(C(F)(F)F)c3OC(=O)N(C)c3ccc(F)cc3)C2=O)C(=O)C1 ; CN(C(=O)Oc1c(N2CCN(CCN3CCNCC3=O)C2=O)cc(C(F)(F)F)cc1C(F)(F)F)c1ccc(F)cc1]

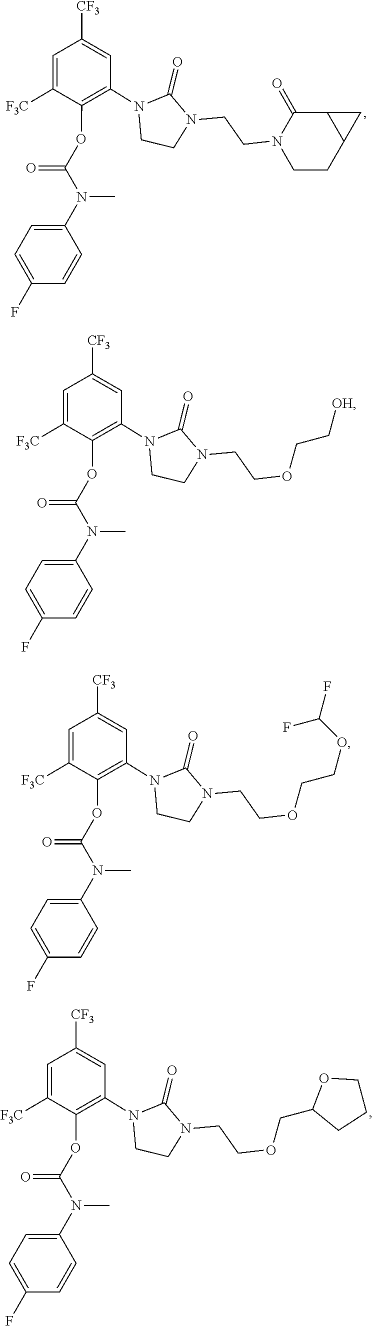 [SMILES: CN(C(=O)Oc1c(N2CCN(CCN3CCC4CC4C3=O)C2=O)cc(C(F)(F)F)cc1C(F)(F)F)c1ccc(F)cc1 ; CN(C(=O)Oc1c(N2CCN(CCOCCO)C2=O)cc(C(F)(F)F)cc1C(F)(F)F)c1ccc(F)cc1 ; CN(C(=O)Oc1c(N2CCN(CCOCCOC(F)F)C2=O)cc(C(F)(F)F)cc1C(F)(F)F)c1ccc(F)cc1 ; CN(C(=O)Oc1c(N2CCN(CCOCC3CCCO3)C2=O)cc(C(F)(F)F)cc1C(F)(F)F)c1ccc(F)cc1]

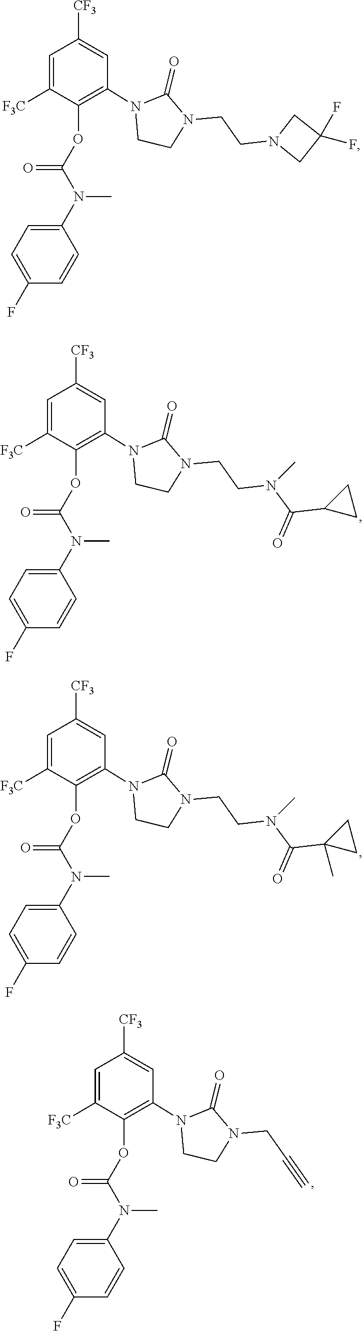 [SMILES: CN(C(=O)Oc1c(N2CCN(CCN3CC(F)(F)C3)C2=O)cc(C(F)(F)F)cc1C(F)(F)F)c1ccc(F)cc1 ; CN(CCN1CCN(c2cc(C(F)(F)F)cc(C(F)(F)F)c2OC(=O)N(C)c2ccc(F)cc2)C1=O)C(=O)C1CC1 ; CN(CCN1CCN(c2cc(C(F)(F)F)cc(C(F)(F)F)c2OC(=O)N(C)c2ccc(F)cc2)C1=O)C(=O)C1(C)CC1 ; C#CCN1CCN(c2cc(C(F)(F)F)cc(C(F)(F)F)c2OC(=O)N(C)c2ccc(F)cc2)C1=O]

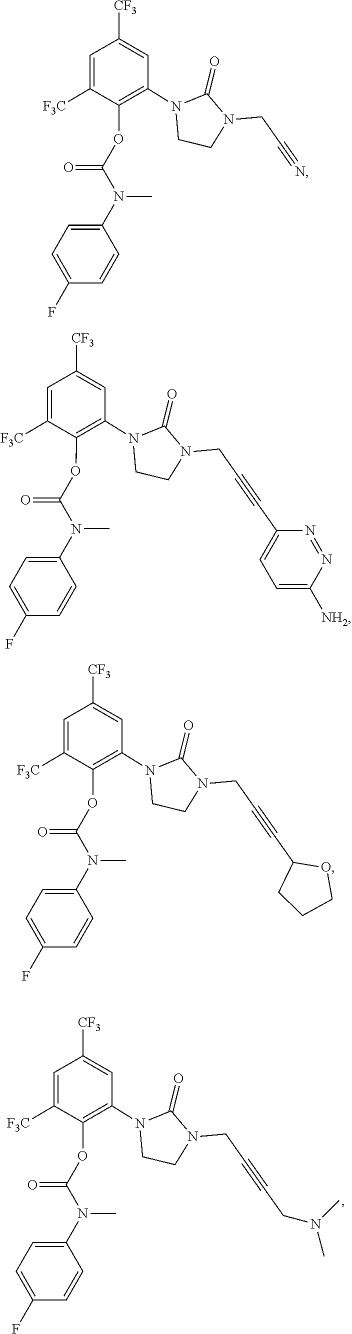

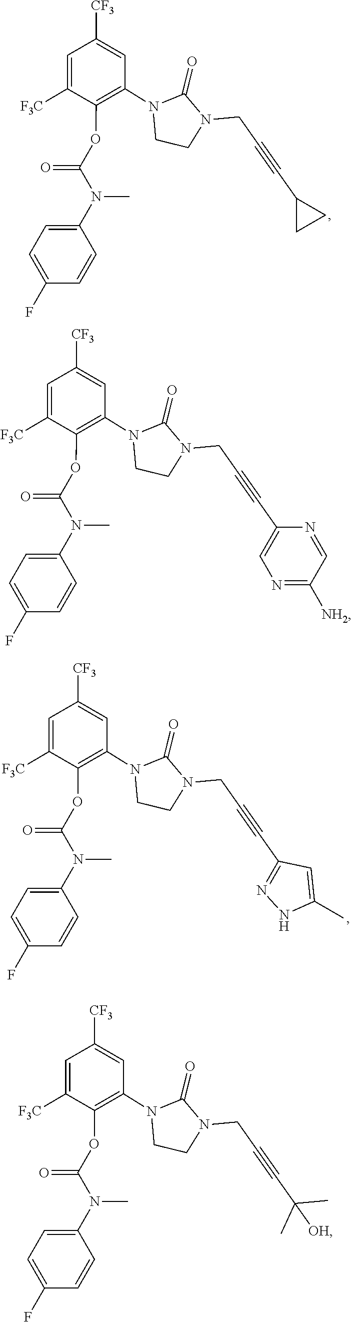 [SMILES: CN(C(=O)Oc1c(N2CCN(CC#CC3CC3)C2=O)cc(C(F)(F)F)cc1C(F)(F)F)c1ccc(F)cc1 ; CN(C(=O)Oc1c(N2CCN(CC#Cc3cnc(N)cn3)C2=O)cc(C(F)(F)F)cc1C(F)(F)F)c1ccc(F)cc1 ; Cc1cc(C#CCN2CCN(c3cc(C(F)(F)F)cc(C(F)(F)F)c3OC(=O)N(C)c3ccc(F)cc3)C2=O)n[nH]1 ; CN(C(=O)Oc1c(N2CCN(CC#CC(C)(C)O)C2=O)cc(C(F)(F)F)cc1C(F)(F)F)c1ccc(F)cc1]

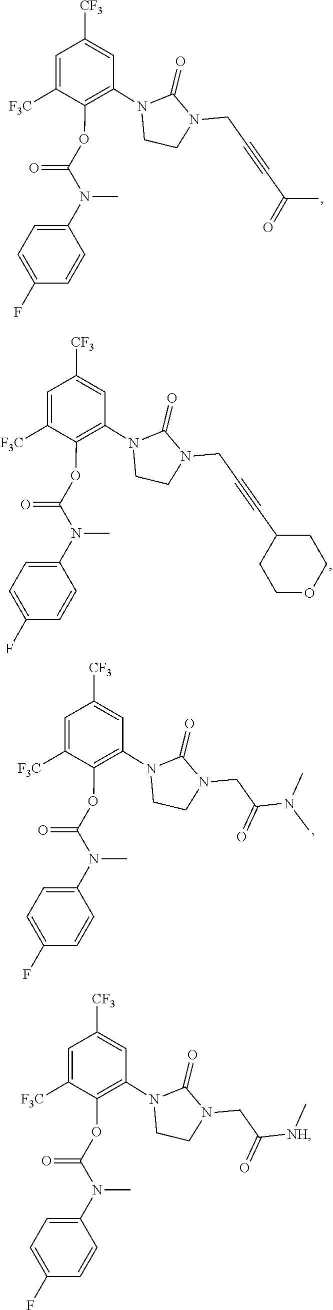 [SMILES: CC(=O)C#CCN1CCN(c2cc(C(F)(F)F)cc(C(F)(F)F)c2OC(=O)N(C)c2ccc(F)cc2)C1=O ; CN(C(=O)Oc1c(N2CCN(CC#CC3CCOCC3)C2=O)cc(C(F)(F)F)cc1C(F)(F)F)c1ccc(F)cc1 ; CN(C)C(=O)CN1CCN(c2cc(C(F)(F)F)cc(C(F)(F)F)c2OC(=O)N(C)c2ccc(F)cc2)C1=O ; CNC(=O)CN1CCN(c2cc(C(F)(F)F)cc(C(F)(F)F)c2OC(=O)N(C)c2ccc(F)cc2)C1=O]

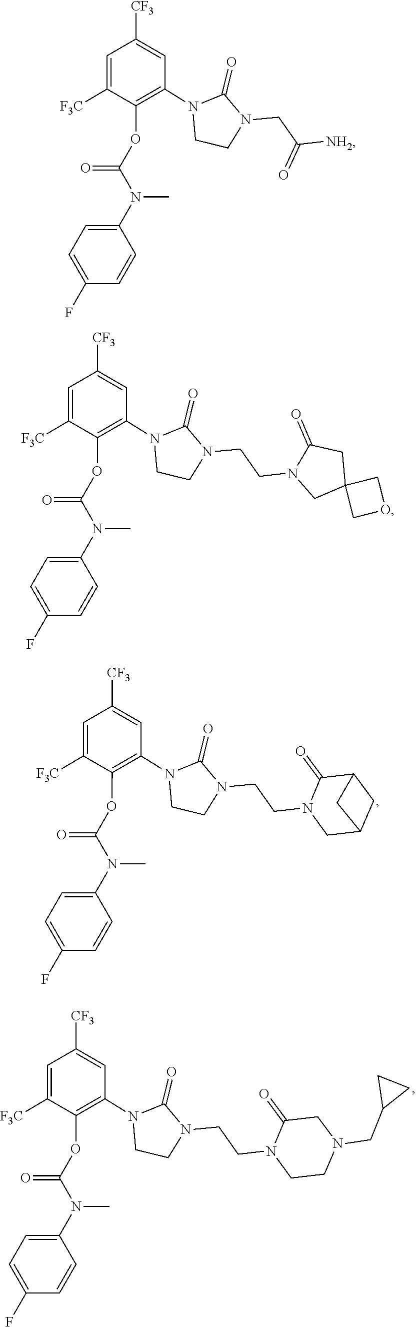

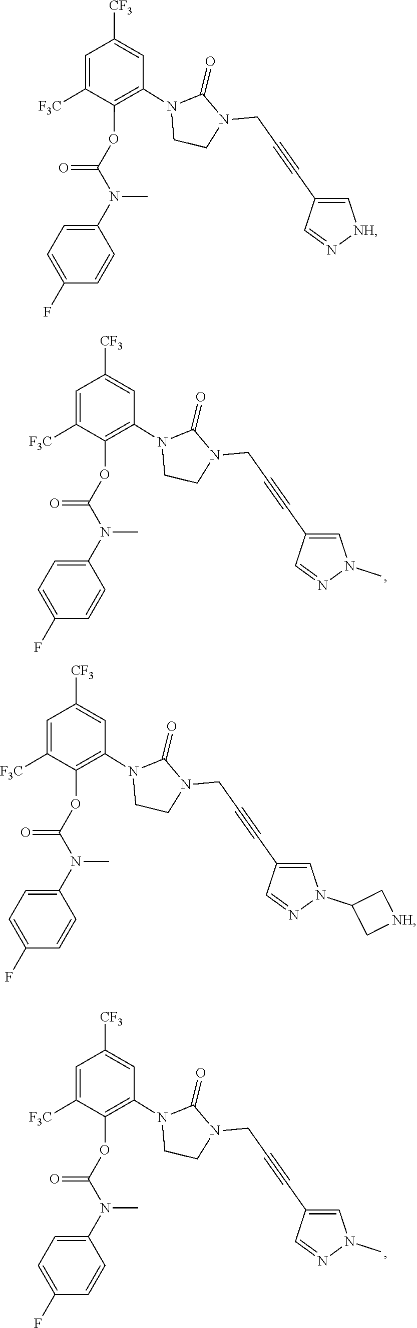

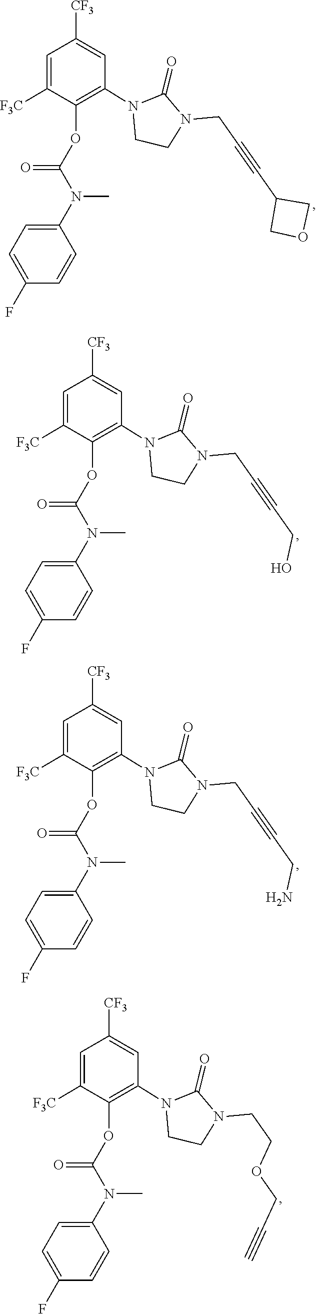 [SMILES: CN(C(=O)Oc1c(N2CCN(CC#CC3COC3)C2=O)cc(C(F)(F)F)cc1C(F)(F)F)c1ccc(F)cc1 ; CN(C(=O)Oc1c(N2CCN(CC#CCO)C2=O)cc(C(F)(F)F)cc1C(F)(F)F)c1ccc(F)cc1 ; CN(C(=O)Oc1c(N2CCN(CC#CCN)C2=O)cc(C(F)(F)F)cc1C(F)(F)F)c1ccc(F)cc1 ; C#CCOCCN1CCN(c2cc(C(F)(F)F)cc(C(F)(F)F)c2OC(=O)N(C)c2ccc(F)cc2)C1=O]

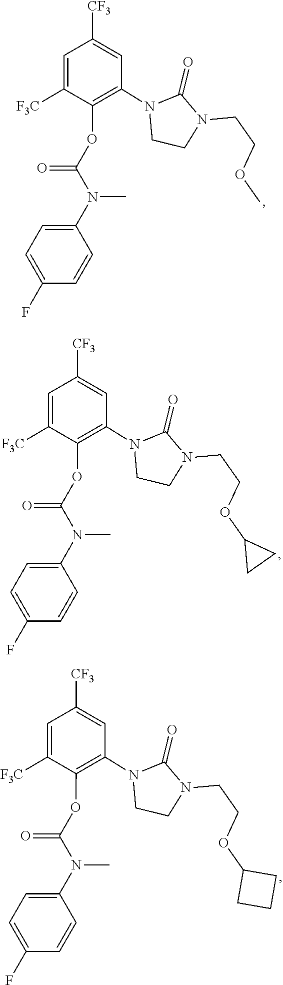 [SMILES: COCCN1CCN(c2cc(C(F)(F)F)cc(C(F)(F)F)c2OC(=O)N(C)c2ccc(F)cc2)C1=O ; CN(C(=O)Oc1c(N2CCN(CCOC3CC3)C2=O)cc(C(F)(F)F)cc1C(F)(F)F)c1ccc(F)cc1 ; CN(C(=O)Oc1c(N2CCN(CCOC3CCC3)C2=O)cc(C(F)(F)F)cc1C(F)(F)F)c1ccc(F)cc1]

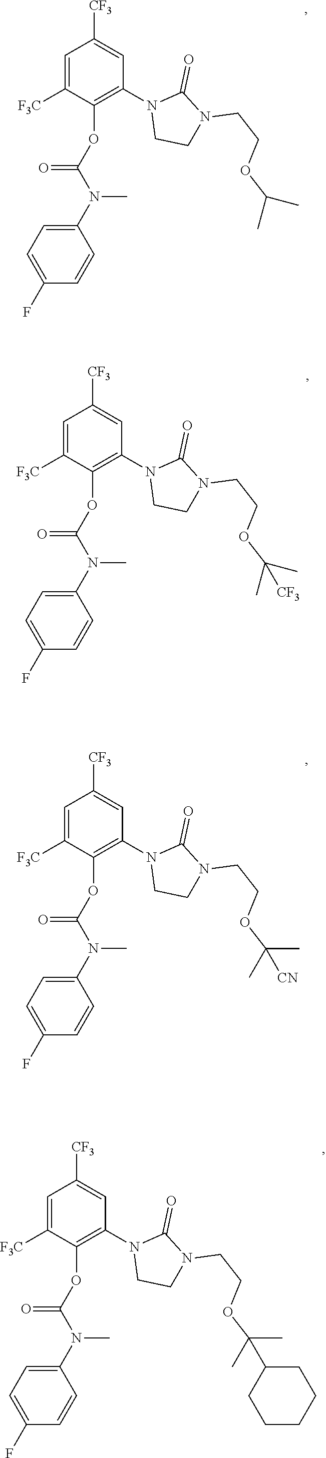 [SMILES: CC(C)OCCN1CCN(c2cc(C(F)(F)F)cc(C(F)(F)F)c2OC(=O)N(C)c2ccc(F)cc2)C1=O ; CN(C(=O)Oc1c(N2CCN(CCOC(C)(C)C(F)(F)F)C2=O)cc(C(F)(F)F)cc1C(F)(F)F)c1ccc(F)cc1 ; CN(C(=O)Oc1c(N2CCN(CCOC(C)(C)C#N)C2=O)cc(C(F)(F)F)cc1C(F)(F)F)c1ccc(F)cc1 ; CN(C(=O)Oc1c(N2CCN(CCOC(C)(C)C3CCCCC3)C2=O)cc(C(F)(F)F)cc1C(F)(F)F)c1ccc(F)cc1]

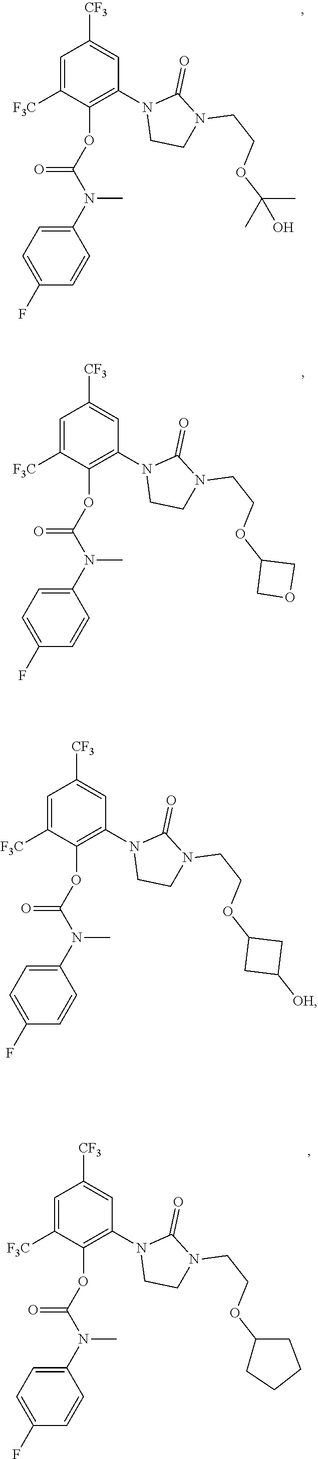 [SMILES: CN(C(=O)Oc1c(N2CCN(CCOC(C)(C)O)C2=O)cc(C(F)(F)F)cc1C(F)(F)F)c1ccc(F)cc1 ; CN(C(=O)Oc1c(N2CCN(CCOC3COC3)C2=O)cc(C(F)(F)F)cc1C(F)(F)F)c1ccc(F)cc1 ; CN(C(=O)Oc1c(N2CCN(CCOC3CC(O)C3)C2=O)cc(C(F)(F)F)cc1C(F)(F)F)c1ccc(F)cc1 ; CN(C(=O)Oc1c(N2CCN(CCOC3CCCC3)C2=O)cc(C(F)(F)F)cc1C(F)(F)F)c1ccc(F)cc1]

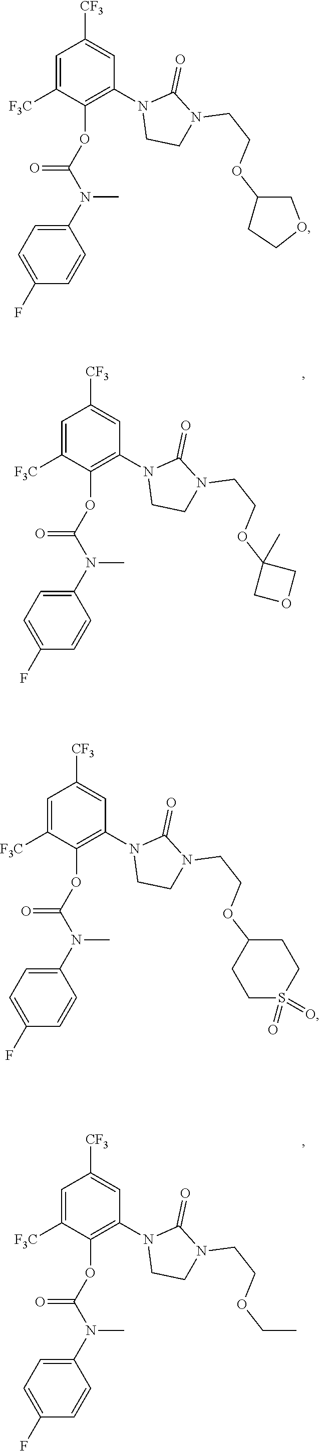 [SMILES: CN(C(=O)Oc1c(N2CCN(CCOC3CCOC3)C2=O)cc(C(F)(F)F)cc1C(F)(F)F)c1ccc(F)cc1 ; CN(C(=O)Oc1c(N2CCN(CCOC3(C)COC3)C2=O)cc(C(F)(F)F)cc1C(F)(F)F)c1ccc(F)cc1 ; CN(C(=O)Oc1c(N2CCN(CCOC3CCS(=O)(=O)CC3)C2=O)cc(C(F)(F)F)cc1C(F)(F)F)c1ccc(F)cc1 ; CCOCCN1CCN(c2cc(C(F)(F)F)cc(C(F)(F)F)c2OC(=O)N(C)c2ccc(F)cc2)C1=O]

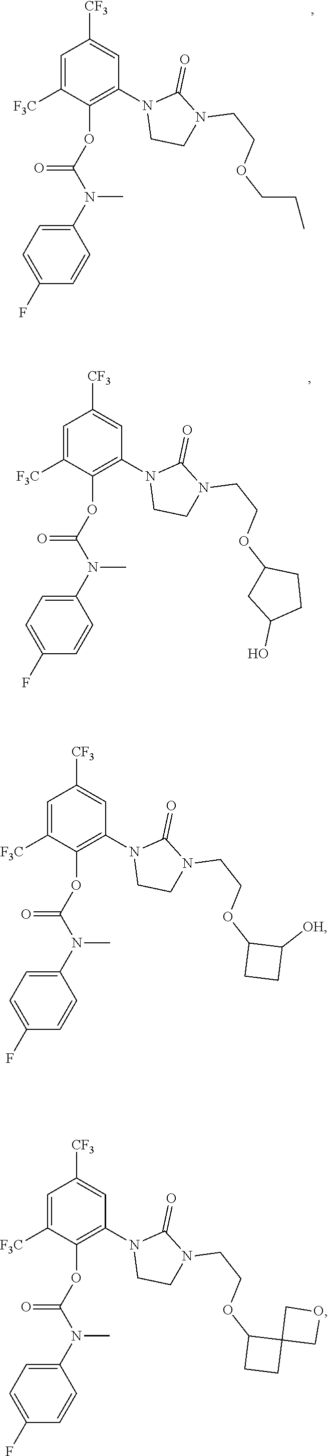 [SMILES: CCCOCCN1CCN(c2cc(C(F)(F)F)cc(C(F)(F)F)c2OC(=O)N(C)c2ccc(F)cc2)C1=O ; CN(C(=O)Oc1c(N2CCN(CCOC3CCC(O)C3)C2=O)cc(C(F)(F)F)cc1C(F)(F)F)c1ccc(F)cc1 ; CN(C(=O)Oc1c(N2CCN(CCOC3CCC3O)C2=O)cc(C(F)(F)F)cc1C(F)(F)F)c1ccc(F)cc1 ; CN(C(=O)Oc1c(N2CCN(CCOC3CCC34COC4)C2=O)cc(C(F)(F)F)cc1C(F)(F)F)c1ccc(F)cc1]

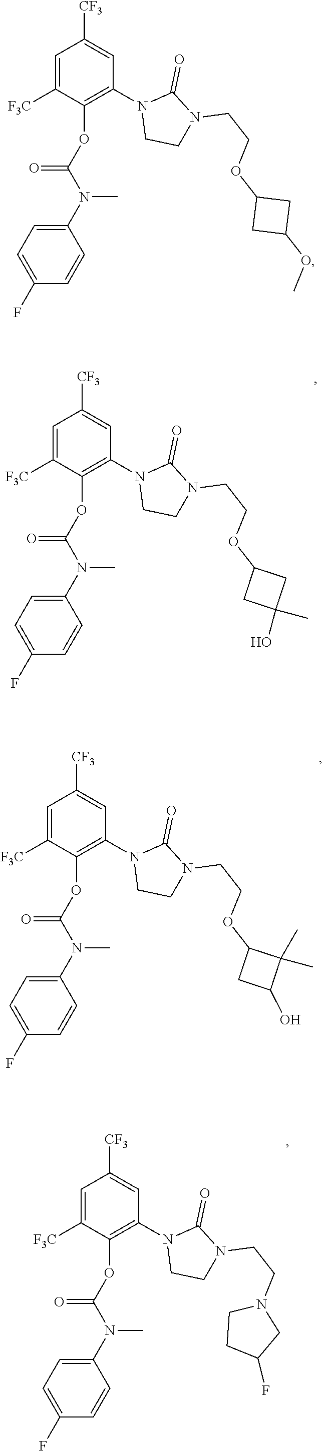 [SMILES: COC1CC(OCCN2CCN(c3cc(C(F)(F)F)cc(C(F)(F)F)c3OC(=O)N(C)c3ccc(F)cc3)C2=O)C1 ; CN(C(=O)Oc1c(N2CCN(CCOC3CC(C)(O)C3)C2=O)cc(C(F)(F)F)cc1C(F)(F)F)c1ccc(F)cc1 ; CN(C(=O)Oc1c(N2CCN(CCOC3CC(O)C3(C)C)C2=O)cc(C(F)(F)F)cc1C(F)(F)F)c1ccc(F)cc1 ; CN(C(=O)Oc1c(N2CCN(CCN3CCC(F)C3)C2=O)cc(C(F)(F)F)cc1C(F)(F)F)c1ccc(F)cc1]

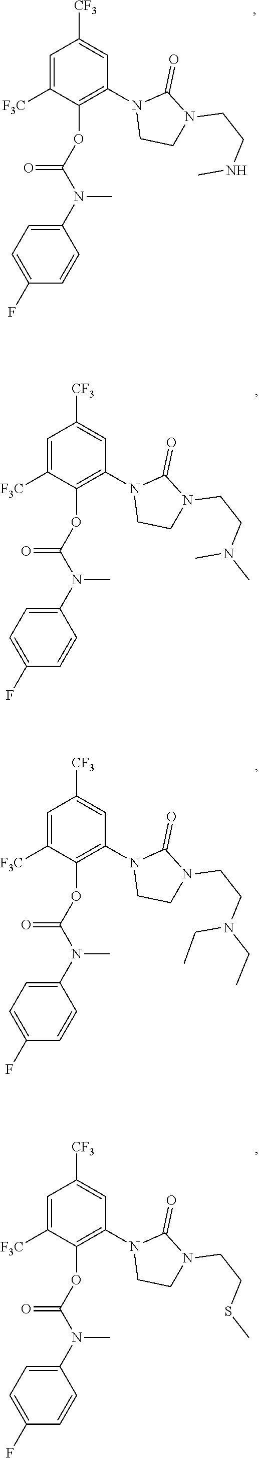 [SMILES: CNCCN1CCN(c2cc(C(F)(F)F)cc(C(F)(F)F)c2OC(=O)N(C)c2ccc(F)cc2)C1=O ; CN(C)CCN1CCN(c2cc(C(F)(F)F)cc(C(F)(F)F)c2OC(=O)N(C)c2ccc(F)cc2)C1=O ; CCN(CC)CCN1CCN(c2cc(C(F)(F)F)cc(C(F)(F)F)c2OC(=O)N(C)c2ccc(F)cc2)C1=O ; CSCCN1CCN(c2cc(C(F)(F)F)cc(C(F)(F)F)c2OC(=O)N(C)c2ccc(F)cc2)C1=O]

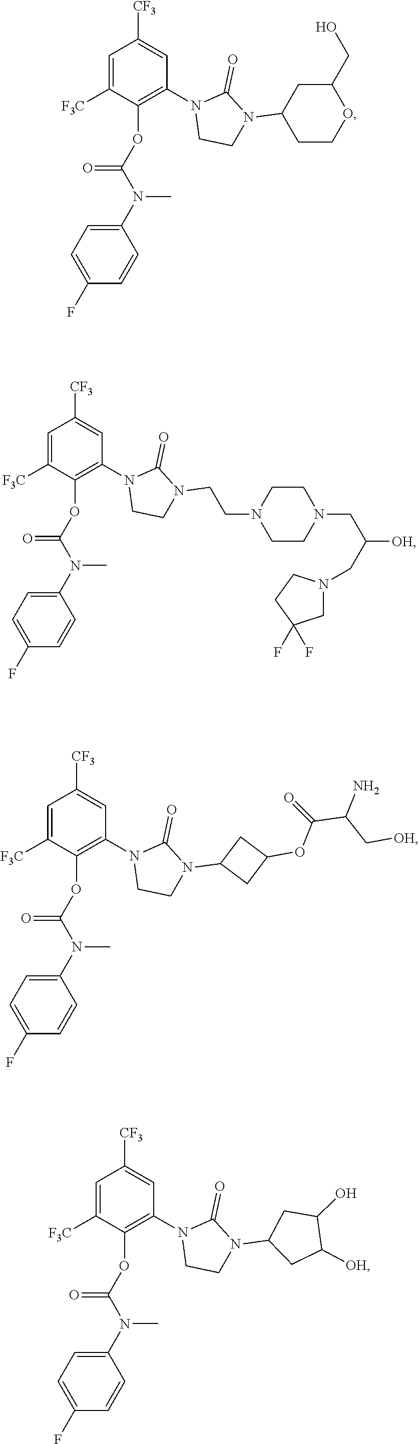 [SMILES: CN(C(=O)Oc1c(N2CCN(C3CCOC(CO)C3)C2=O)cc(C(F)(F)F)cc1C(F)(F)F)c1ccc(F)cc1 ; CN(C(=O)Oc1c(N2CCN(CCN3CCN(CC(O)CN4CCC(F)(F)C4)CC3)C2=O)cc(C(F)(F)F)cc1C(F)(F)F)c1ccc(F)cc1 ; CN(C(=O)Oc1c(N2CCN(C3CC(OC(=O)C(N)CO)C3)C2=O)cc(C(F)(F)F)cc1C(F)(F)F)c1ccc(F)cc1 ; CN(C(=O)Oc1c(N2CCN(C3CC(O)C(O)C3)C2=O)cc(C(F)(F)F)cc1C(F)(F)F)c1ccc(F)cc1]

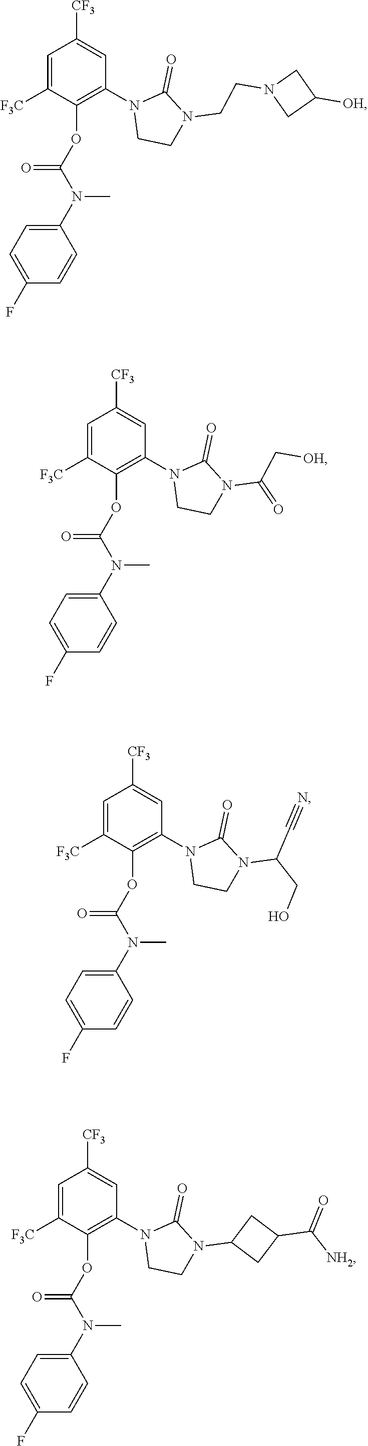 [SMILES: CN(C(=O)Oc1c(N2CCN(CCN3CC(O)C3)C2=O)cc(C(F)(F)F)cc1C(F)(F)F)c1ccc(F)cc1 ; CN(C(=O)Oc1c(N2CCN(C(=O)CO)C2=O)cc(C(F)(F)F)cc1C(F)(F)F)c1ccc(F)cc1 ; CN(C(=O)Oc1c(N2CCN(C(C#N)CO)C2=O)cc(C(F)(F)F)cc1C(F)(F)F)c1ccc(F)cc1 ; CN(C(=O)Oc1c(N2CCN(C3CC(C(N)=O)C3)C2=O)cc(C(F)(F)F)cc1C(F)(F)F)c1ccc(F)cc1]

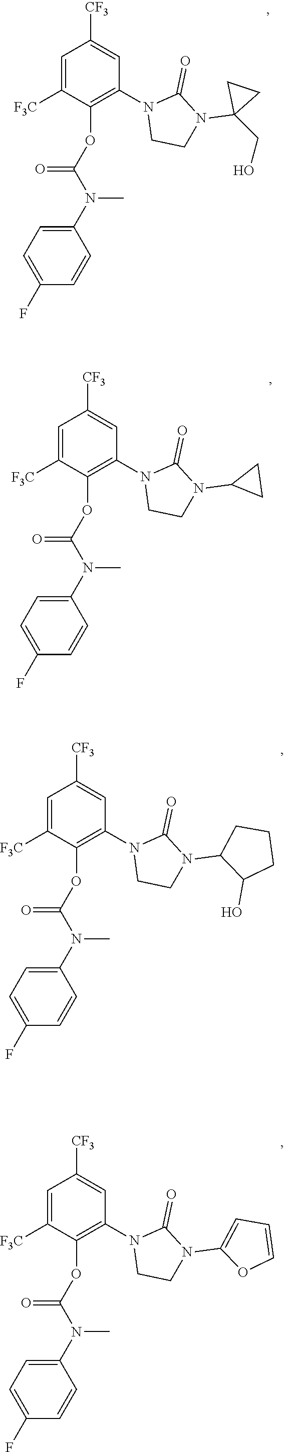 [SMILES: CN(C(=O)Oc1c(N2CCN(C3(CO)CC3)C2=O)cc(C(F)(F)F)cc1C(F)(F)F)c1ccc(F)cc1 ; CN(C(=O)Oc1c(N2CCN(C3CC3)C2=O)cc(C(F)(F)F)cc1C(F)(F)F)c1ccc(F)cc1 ; CN(C(=O)Oc1c(N2CCN(C3CCCC3O)C2=O)cc(C(F)(F)F)cc1C(F)(F)F)c1ccc(F)cc1 ; CN(C(=O)Oc1c(N2CCN(c3ccco3)C2=O)cc(C(F)(F)F)cc1C(F)(F)F)c1ccc(F)cc1]

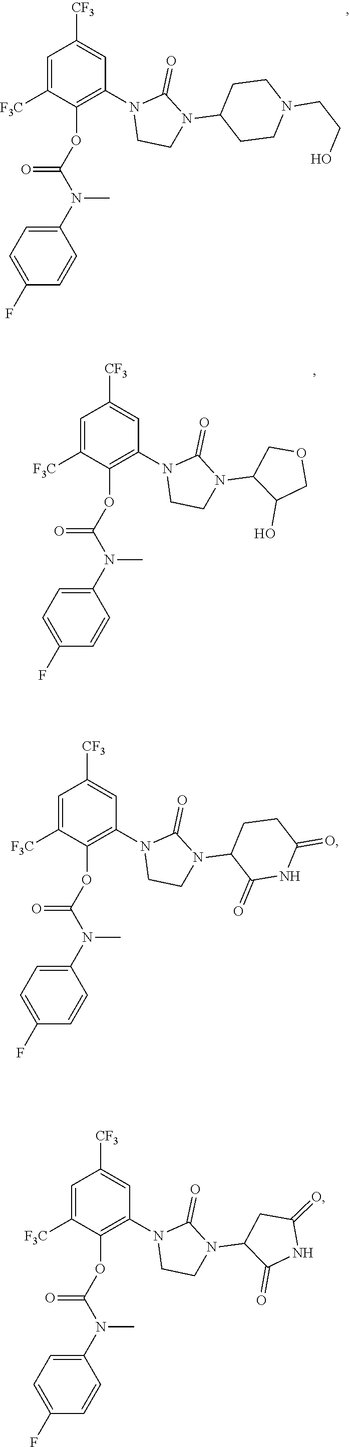 [SMILES: CN(C(=O)Oc1c(N2CCN(C3CCN(CCO)CC3)C2=O)cc(C(F)(F)F)cc1C(F)(F)F)c1ccc(F)cc1 ; CN(C(=O)Oc1c(N2CCN(C3COCC3O)C2=O)cc(C(F)(F)F)cc1C(F)(F)F)c1ccc(F)cc1 ; CN(C(=O)Oc1c(N2CCN(C3CCC(=O)NC3=O)C2=O)cc(C(F)(F)F)cc1C(F)(F)F)c1ccc(F)cc1 ; CN(C(=O)Oc1c(N2CCN(C3CC(=O)NC3=O)C2=O)cc(C(F)(F)F)cc1C(F)(F)F)c1ccc(F)cc1]

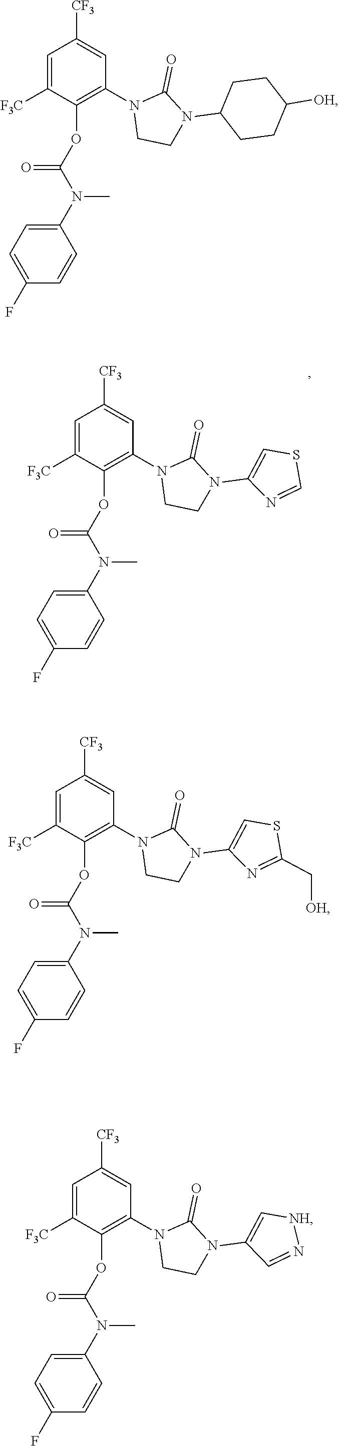 [SMILES: CN(C(=O)Oc1c(N2CCN(C3CCC(O)CC3)C2=O)cc(C(F)(F)F)cc1C(F)(F)F)c1ccc(F)cc1 ; CN(C(=O)Oc1c(N2CCN(c3cscn3)C2=O)cc(C(F)(F)F)cc1C(F)(F)F)c1ccc(F)cc1 ; CN(C(=O)Oc1c(N2CCN(c3csc(CO)n3)C2=O)cc(C(F)(F)F)cc1C(F)(F)F)c1ccc(F)cc1 ; CN(C(=O)Oc1c(N2CCN(c3cn[nH]c3)C2=O)cc(C(F)(F)F)cc1C(F)(F)F)c1ccc(F)cc1]

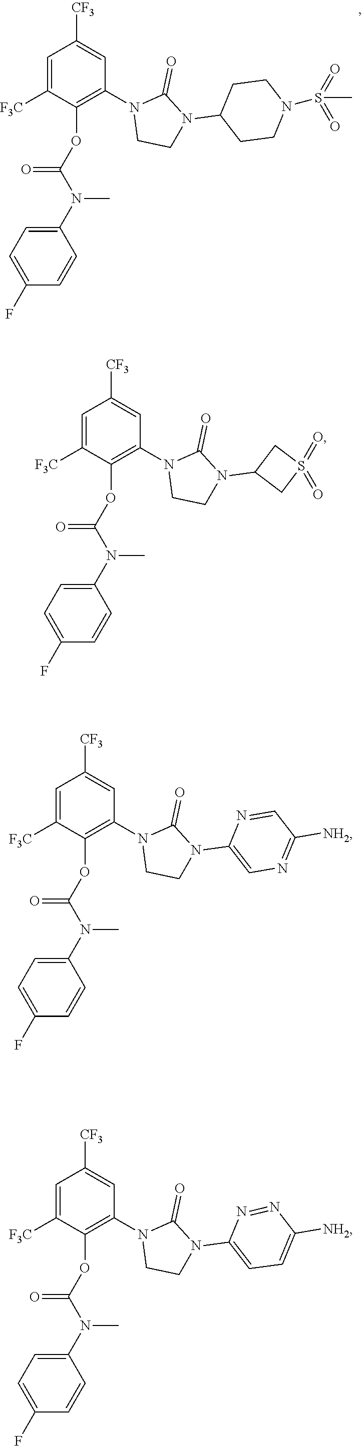 [SMILES: CN(C(=O)Oc1c(N2CCN(C3CCN(S(C)(=O)=O)CC3)C2=O)cc(C(F)(F)F)cc1C(F)(F)F)c1ccc(F)cc1 ; CN(C(=O)Oc1c(N2CCN(C3CS(=O)(=O)C3)C2=O)cc(C(F)(F)F)cc1C(F)(F)F)c1ccc(F)cc1 ; CN(C(=O)Oc1c(N2CCN(c3cnc(N)cn3)C2=O)cc(C(F)(F)F)cc1C(F)(F)F)c1ccc(F)cc1 ; CN(C(=O)Oc1c(N2CCN(c3ccc(N)nn3)C2=O)cc(C(F)(F)F)cc1C(F)(F)F)c1ccc(F)cc1]

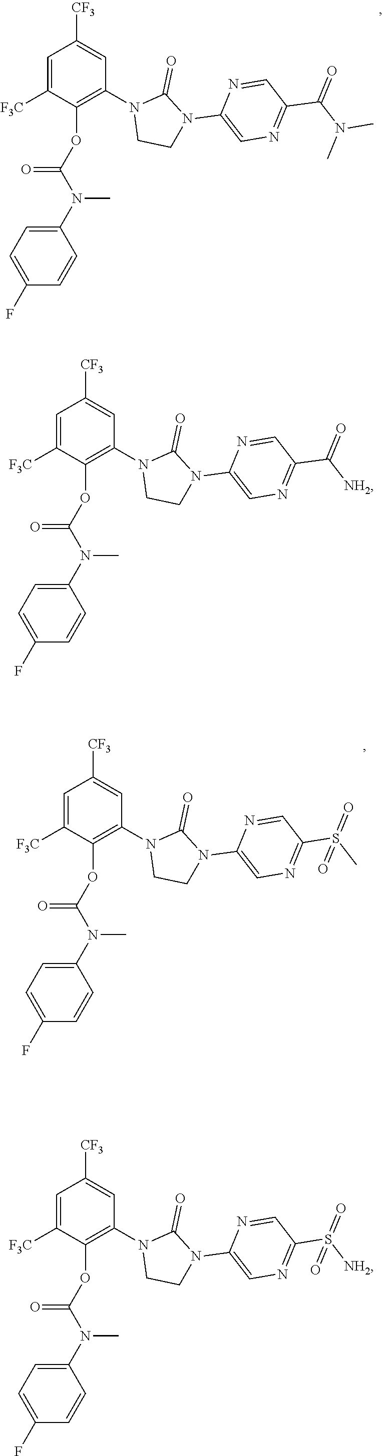 [SMILES: CN(C)C(=O)c1cnc(N2CCN(c3cc(C(F)(F)F)cc(C(F)(F)F)c3OC(=O)N(C)c3ccc(F)cc3)C2=O)cn1 ; CN(C(=O)Oc1c(N2CCN(c3cnc(C(N)=O)cn3)C2=O)cc(C(F)(F)F)cc1C(F)(F)F)c1ccc(F)cc1 ; CN(C(=O)Oc1c(N2CCN(c3cnc(S(C)(=O)=O)cn3)C2=O)cc(C(F)(F)F)cc1C(F)(F)F)c1ccc(F)cc1 ; CN(C(=O)Oc1c(N2CCN(c3cnc(S(N)(=O)=O)cn3)C2=O)cc(C(F)(F)F)cc1C(F)(F)F)c1ccc(F)cc1]

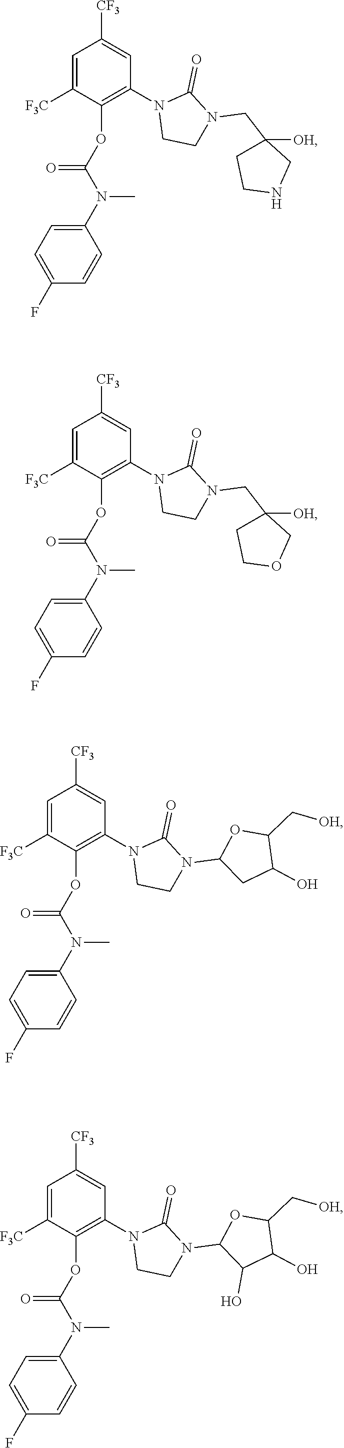 [SMILES: CN(C(=O)Oc1c(N2CCN(CC3(O)CCNC3)C2=O)cc(C(F)(F)F)cc1C(F)(F)F)c1ccc(F)cc1 ; CN(C(=O)Oc1c(N2CCN(CC3(O)CCOC3)C2=O)cc(C(F)(F)F)cc1C(F)(F)F)c1ccc(F)cc1 ; CN(C(=O)Oc1c(N2CCN(C3CC(O)C(CO)O3)C2=O)cc(C(F)(F)F)cc1C(F)(F)F)c1ccc(F)cc1 ; CN(C(=O)Oc1c(N2CCN(C3OC(CO)C(O)C3O)C2=O)cc(C(F)(F)F)cc1C(F)(F)F)c1ccc(F)cc1]

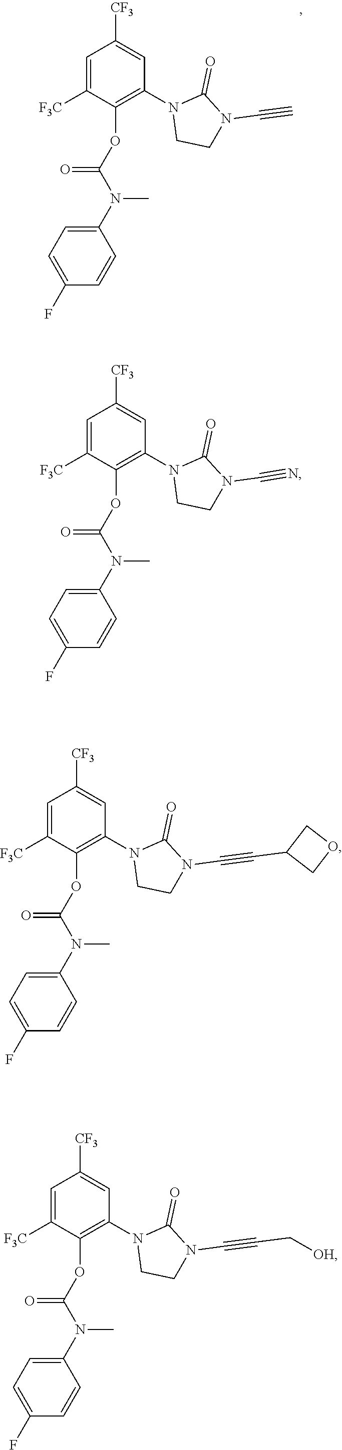 [SMILES: C#CN1CCN(c2cc(C(F)(F)F)cc(C(F)(F)F)c2OC(=O)N(C)c2ccc(F)cc2)C1=O ; CN(C(=O)Oc1c(N2CCN(C#N)C2=O)cc(C(F)(F)F)cc1C(F)(F)F)c1ccc(F)cc1 ; CN(C(=O)Oc1c(N2CCN(C#CC3COC3)C2=O)cc(C(F)(F)F)cc1C(F)(F)F)c1ccc(F)cc1 ; CN(C(=O)Oc1c(N2CCN(C#CCO)C2=O)cc(C(F)(F)F)cc1C(F)(F)F)c1ccc(F)cc1]

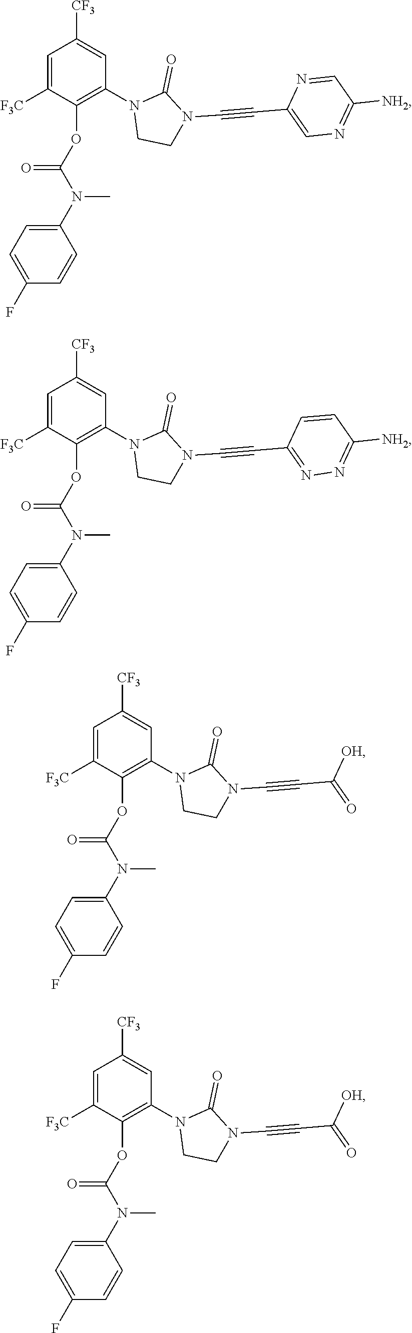 [SMILES: CN(C(=O)Oc1c(N2CCN(C#Cc3cnc(N)cn3)C2=O)cc(C(F)(F)F)cc1C(F)(F)F)c1ccc(F)cc1 ; CN(C(=O)Oc1c(N2CCN(C#Cc3ccc(N)nn3)C2=O)cc(C(F)(F)F)cc1C(F)(F)F)c1ccc(F)cc1 ; CN(C(=O)Oc1c(N2CCN(C#CC(=O)O)C2=O)cc(C(F)(F)F)cc1C(F)(F)F)c1ccc(F)cc1 ; CN(C(=O)Oc1c(N2CCN(C#CC(=O)O)C2=O)cc(C(F)(F)F)cc1C(F)(F)F)c1ccc(F)cc1]

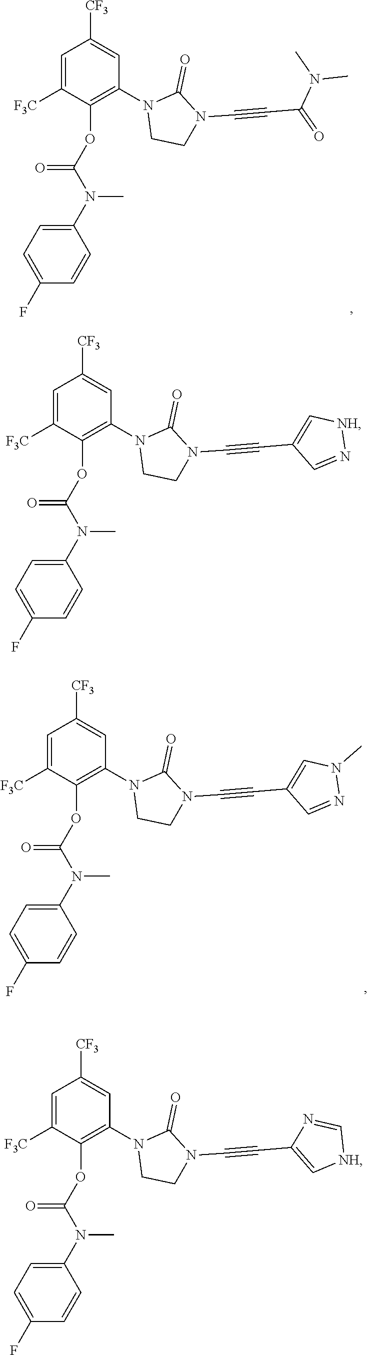 [SMILES: CN(C)C(=O)C#CN1CCN(c2cc(C(F)(F)F)cc(C(F)(F)F)c2OC(=O)N(C)c2ccc(F)cc2)C1=O ; CN(C(=O)Oc1c(N2CCN(C#Cc3cn[nH]c3)C2=O)cc(C(F)(F)F)cc1C(F)(F)F)c1ccc(F)cc1 ; CN(C(=O)Oc1c(N2CCN(C#Cc3cnn(C)c3)C2=O)cc(C(F)(F)F)cc1C(F)(F)F)c1ccc(F)cc1 ; CN(C(=O)Oc1c(N2CCN(C#Cc3c[nH]cn3)C2=O)cc(C(F)(F)F)cc1C(F)(F)F)c1ccc(F)cc1]

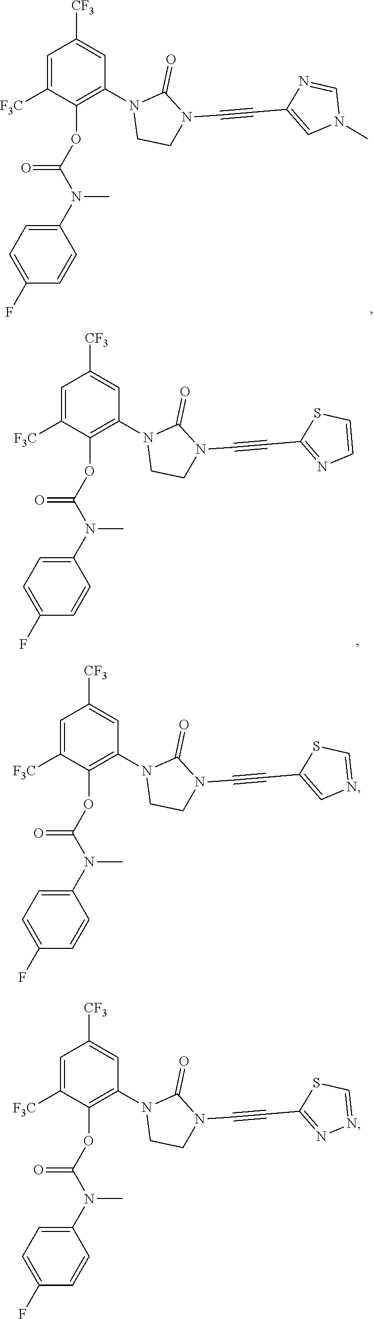 [SMILES: CN(C(=O)Oc1c(N2CCN(C#Cc3cn(C)cn3)C2=O)cc(C(F)(F)F)cc1C(F)(F)F)c1ccc(F)cc1 ; CN(C(=O)Oc1c(N2CCN(C#Cc3nccs3)C2=O)cc(C(F)(F)F)cc1C(F)(F)F)c1ccc(F)cc1 ; CN(C(=O)Oc1c(N2CCN(C#Cc3cncs3)C2=O)cc(C(F)(F)F)cc1C(F)(F)F)c1ccc(F)cc1 ; CN(C(=O)Oc1c(N2CCN(C#Cc3nncs3)C2=O)cc(C(F)(F)F)cc1C(F)(F)F)c1ccc(F)cc1]

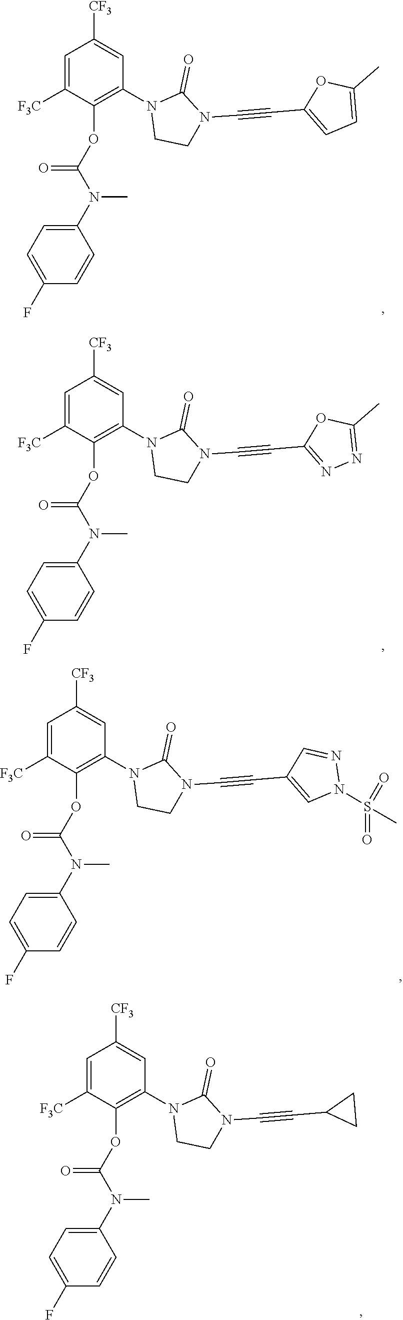 [SMILES: Cc1ccc(C#CN2CCN(c3cc(C(F)(F)F)cc(C(F)(F)F)c3OC(=O)N(C)c3ccc(F)cc3)C2=O)o1 ; Cc1nnc(C#CN2CCN(c3cc(C(F)(F)F)cc(C(F)(F)F)c3OC(=O)N(C)c3ccc(F)cc3)C2=O)o1 ; CN(C(=O)Oc1c(N2CCN(C#Cc3cnn(S(C)(=O)=O)c3)C2=O)cc(C(F)(F)F)cc1C(F)(F)F)c1ccc(F)cc1 ; CN(C(=O)Oc1c(N2CCN(C#CC3CC3)C2=O)cc(C(F)(F)F)cc1C(F)(F)F)c1ccc(F)cc1]

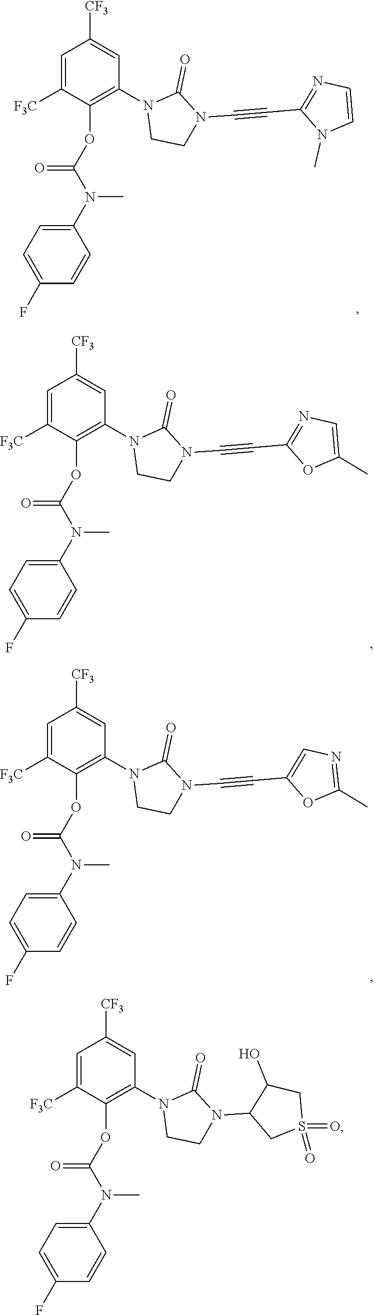 [SMILES: CN(C(=O)Oc1c(N2CCN(C#Cc3nccn3C)C2=O)cc(C(F)(F)F)cc1C(F)(F)F)c1ccc(F)cc1 ; Cc1cnc(C#CN2CCN(c3cc(C(F)(F)F)cc(C(F)(F)F)c3OC(=O)N(C)c3ccc(F)cc3)C2=O)o1 ; Cc1ncc(C#CN2CCN(c3cc(C(F)(F)F)cc(C(F)(F)F)c3OC(=O)N(C)c3ccc(F)cc3)C2=O)o1 ; CN(C(=O)Oc1c(N2CCN(C3CS(=O)(=O)CC3O)C2=O)cc(C(F)(F)F)cc1C(F)(F)F)c1ccc(F)cc1]

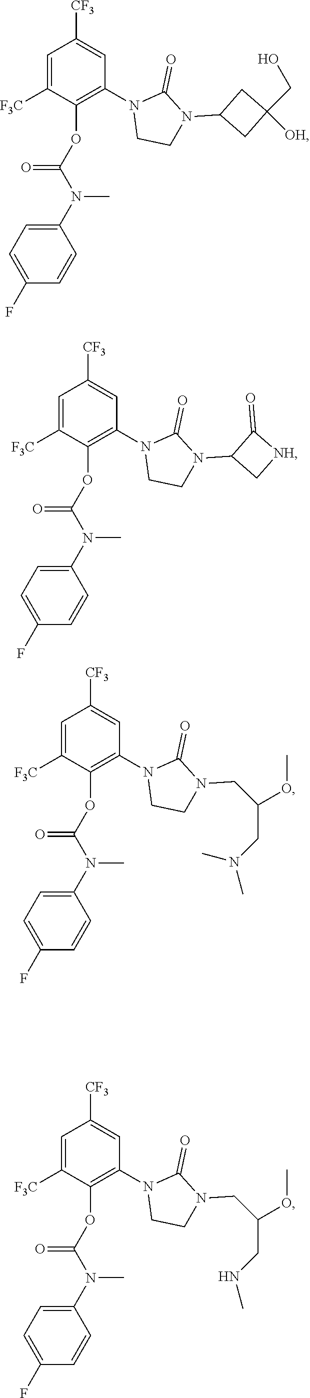 [SMILES: CN(C(=O)Oc1c(N2CCN(C3CC(O)(CO)C3)C2=O)cc(C(F)(F)F)cc1C(F)(F)F)c1ccc(F)cc1 ; CN(C(=O)Oc1c(N2CCN(C3CNC3=O)C2=O)cc(C(F)(F)F)cc1C(F)(F)F)c1ccc(F)cc1 ; COC(CN(C)C)CN1CCN(c2cc(C(F)(F)F)cc(C(F)(F)F)c2OC(=O)N(C)c2ccc(F)cc2)C1=O ; CNCC(CN1CCN(c2cc(C(F)(F)F)cc(C(F)(F)F)c2OC(=O)N(C)c2ccc(F)cc2)C1=O)OC]

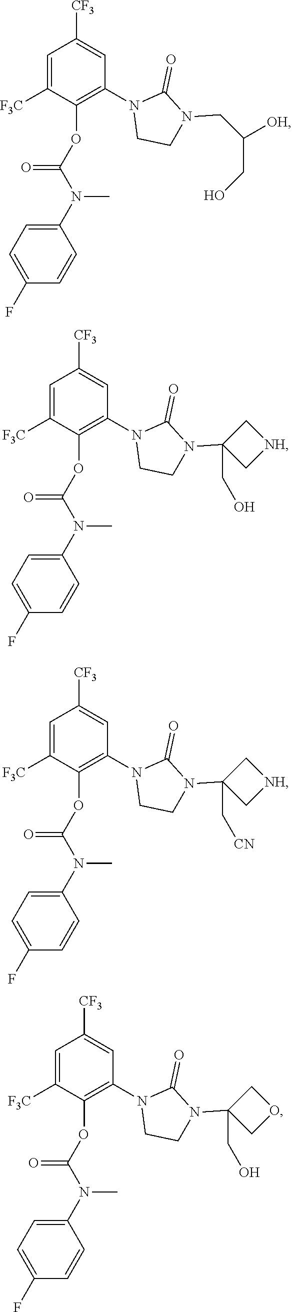 [SMILES: CN(C(=O)Oc1c(N2CCN(CC(O)CO)C2=O)cc(C(F)(F)F)cc1C(F)(F)F)c1ccc(F)cc1 ; CN(C(=O)Oc1c(N2CCN(C3(CO)CNC3)C2=O)cc(C(F)(F)F)cc1C(F)(F)F)c1ccc(F)cc1 ; CN(C(=O)Oc1c(N2CCN(C3(CC#N)CNC3)C2=O)cc(C(F)(F)F)cc1C(F)(F)F)c1ccc(F)cc1 ; CN(C(=O)Oc1c(N2CCN(C3(CO)COC3)C2=O)cc(C(F)(F)F)cc1C(F)(F)F)c1ccc(F)cc1]

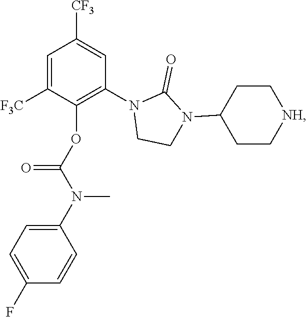 [SMILES: CN(C(=O)Oc1c(N2CCN(C3CCNCC3)C2=O)cc(C(F)(F)F)cc1C(F)(F)F)c1ccc(F)cc1]

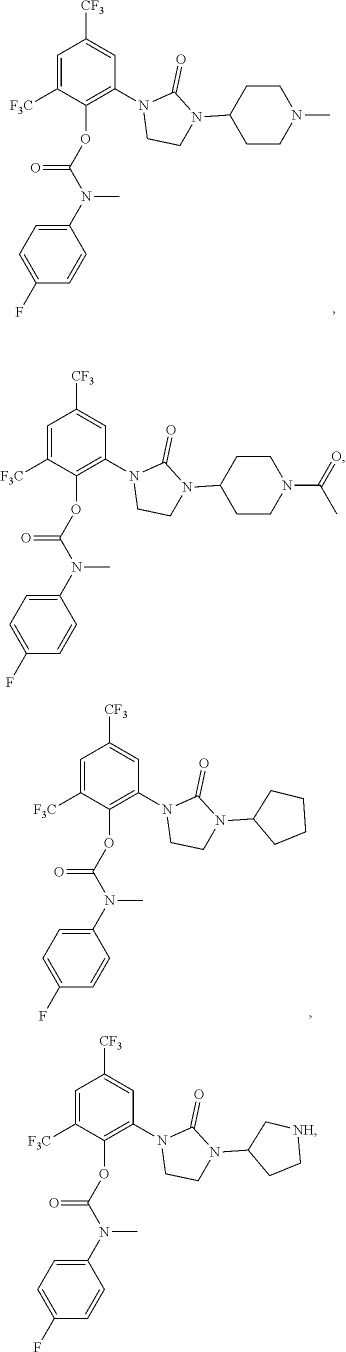 [SMILES: CN1CCC(N2CCN(c3cc(C(F)(F)F)cc(C(F)(F)F)c3OC(=O)N(C)c3ccc(F)cc3)C2=O)CC1 ; CC(=O)N1CCC(N2CCN(c3cc(C(F)(F)F)cc(C(F)(F)F)c3OC(=O)N(C)c3ccc(F)cc3)C2=O)CC1 ; CN(C(=O)Oc1c(N2CCN(C3CCCC3)C2=O)cc(C(F)(F)F)cc1C(F)(F)F)c1ccc(F)cc1 ; CN(C(=O)Oc1c(N2CCN(C3CCNC3)C2=O)cc(C(F)(F)F)cc1C(F)(F)F)c1ccc(F)cc1]

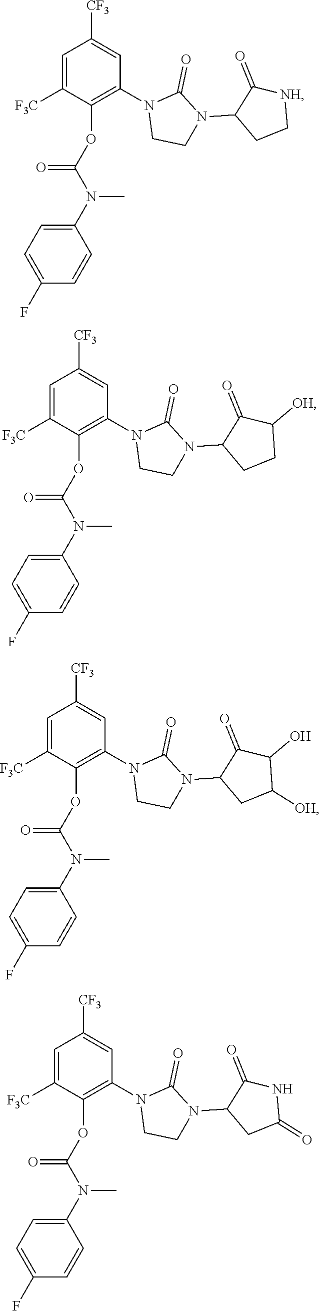 [SMILES: CN(C(=O)Oc1c(N2CCN(C3CCNC3=O)C2=O)cc(C(F)(F)F)cc1C(F)(F)F)c1ccc(F)cc1 ; CN(C(=O)Oc1c(N2CCN(C3CCC(O)C3=O)C2=O)cc(C(F)(F)F)cc1C(F)(F)F)c1ccc(F)cc1 ; CN(C(=O)Oc1c(N2CCN(C3CC(O)C(O)C3=O)C2=O)cc(C(F)(F)F)cc1C(F)(F)F)c1ccc(F)cc1 ; CN(C(=O)Oc1c(N2CCN(C3CC(=O)NC3=O)C2=O)cc(C(F)(F)F)cc1C(F)(F)F)c1ccc(F)cc1]

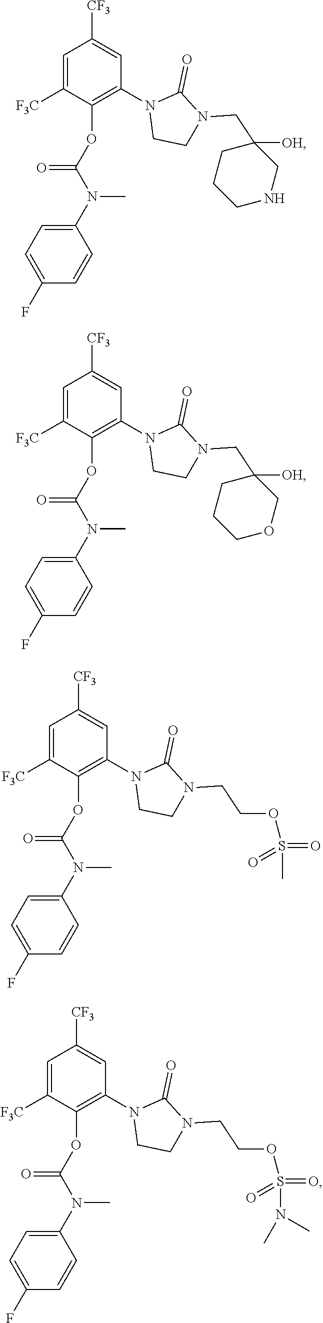 [SMILES: CN(C(=O)Oc1c(N2CCN(CC3(O)CCCNC3)C2=O)cc(C(F)(F)F)cc1C(F)(F)F)c1ccc(F)cc1 ; CN(C(=O)Oc1c(N2CCN(CC3(O)CCCOC3)C2=O)cc(C(F)(F)F)cc1C(F)(F)F)c1ccc(F)cc1 ; CN(C(=O)Oc1c(N2CCN(CCOS(C)(=O)=O)C2=O)cc(C(F)(F)F)cc1C(F)(F)F)c1ccc(F)cc1 ; CN(C(=O)Oc1c(N2CCN(CCOS(=O)(=O)N(C)C)C2=O)cc(C(F)(F)F)cc1C(F)(F)F)c1ccc(F)cc1]

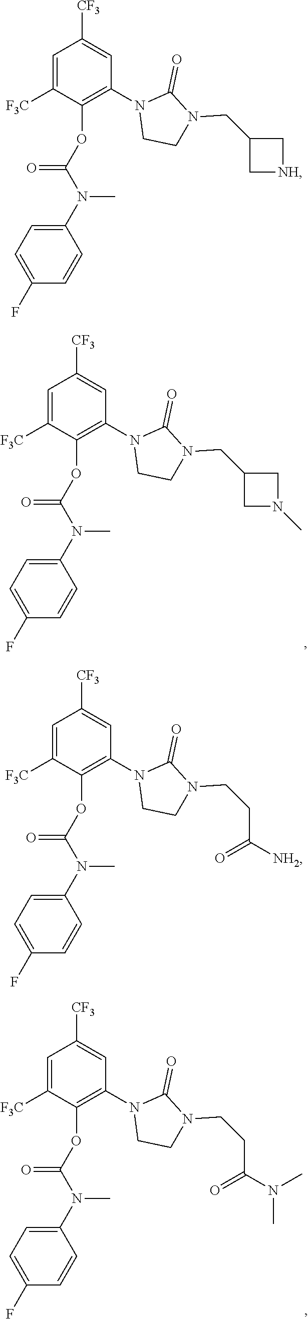 [SMILES: CN(C(=O)Oc1c(N2CCN(CC3CNC3)C2=O)cc(C(F)(F)F)cc1C(F)(F)F)c1ccc(F)cc1 ; CN1CC(CN2CCN(c3cc(C(F)(F)F)cc(C(F)(F)F)c3OC(=O)N(C)c3ccc(F)cc3)C2=O)C1 ; CN(C(=O)Oc1c(N2CCN(CCC(N)=O)C2=O)cc(C(F)(F)F)cc1C(F)(F)F)c1ccc(F)cc1 ; CN(C)C(=O)CCN1CCN(c2cc(C(F)(F)F)cc(C(F)(F)F)c2OC(=O)N(C)c2ccc(F)cc2)C1=O]

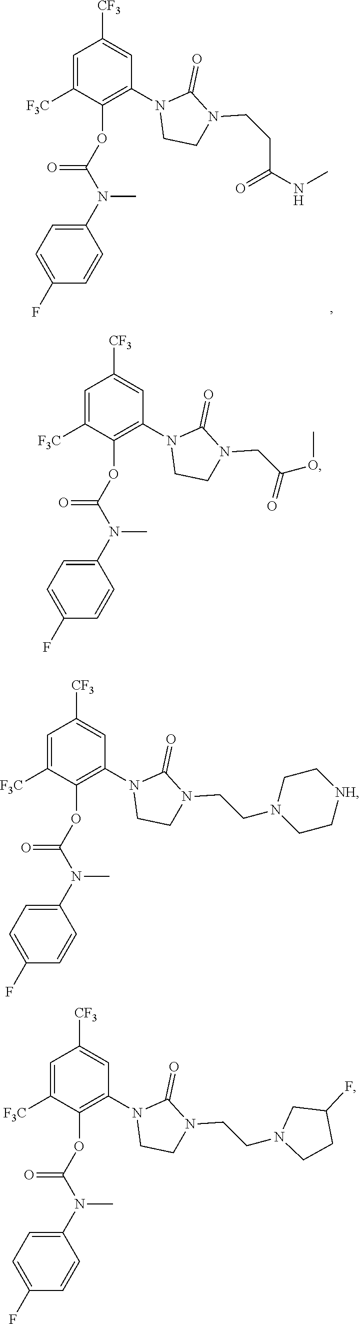 [SMILES: CNC(=O)CCN1CCN(c2cc(C(F)(F)F)cc(C(F)(F)F)c2OC(=O)N(C)c2ccc(F)cc2)C1=O ; COC(=O)CN1CCN(c2cc(C(F)(F)F)cc(C(F)(F)F)c2OC(=O)N(C)c2ccc(F)cc2)C1=O ; CN(C(=O)Oc1c(N2CCN(CCN3CCNCC3)C2=O)cc(C(F)(F)F)cc1C(F)(F)F)c1ccc(F)cc1 ; CN(C(=O)Oc1c(N2CCN(CCN3CCC(F)C3)C2=O)cc(C(F)(F)F)cc1C(F)(F)F)c1ccc(F)cc1]

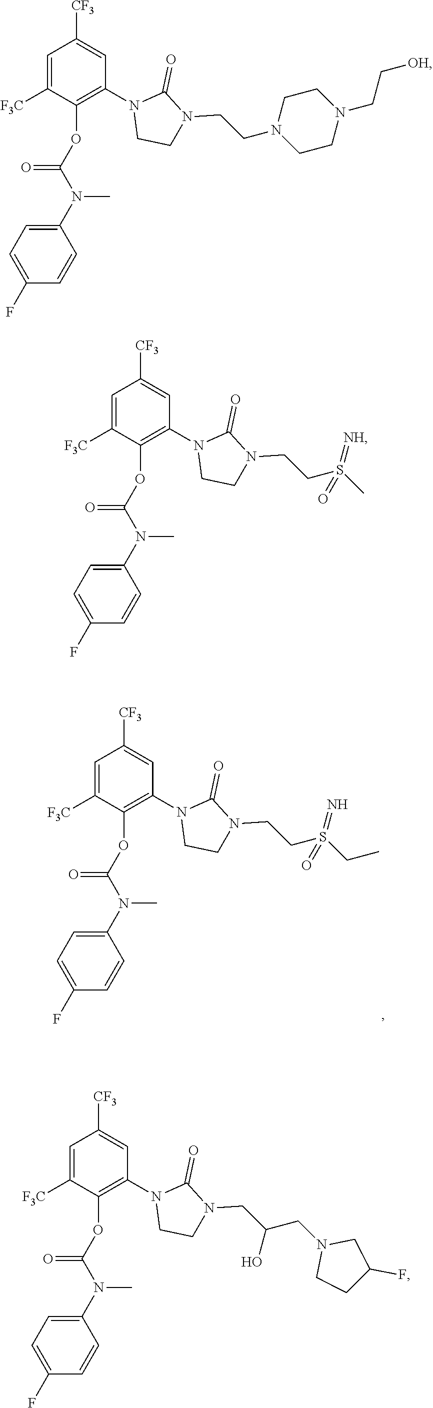 [SMILES: CN(C(=O)Oc1c(N2CCN(CCN3CCN(CCO)CC3)C2=O)cc(C(F)(F)F)cc1C(F)(F)F)c1ccc(F)cc1 ; CN(C(=O)Oc1c(N2CCN(CCS(C)(=N)=O)C2=O)cc(C(F)(F)F)cc1C(F)(F)F)c1ccc(F)cc1 ; CCS(=N)(=O)CCN1CCN(c2cc(C(F)(F)F)cc(C(F)(F)F)c2OC(=O)N(C)c2ccc(F)cc2)C1=O ; CN(C(=O)Oc1c(N2CCN(CC(O)CN3CCC(F)C3)C2=O)cc(C(F)(F)F)cc1C(F)(F)F)c1ccc(F)cc1]

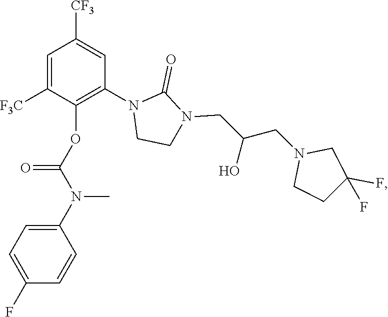 [SMILES: CN(C(=O)Oc1c(N2CCN(CC(O)CN3CCC(F)(F)C3)C2=O)cc(C(F)(F)F)cc1C(F)(F)F)c1ccc(F)cc1]

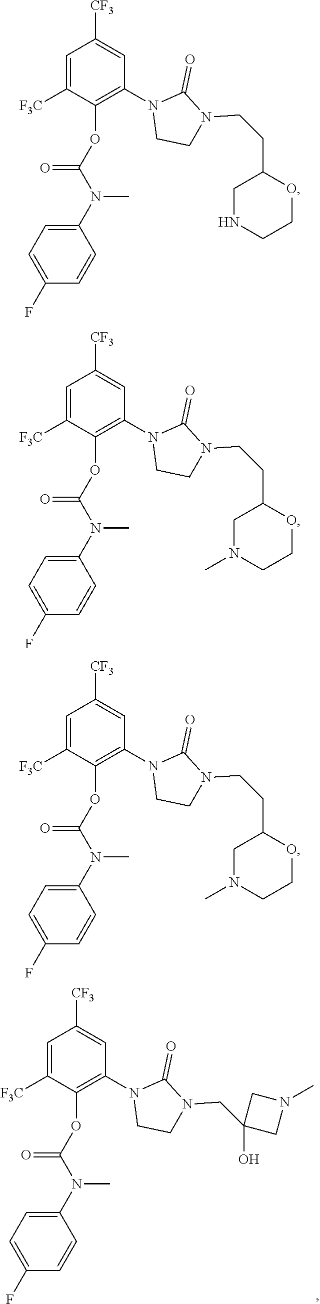 [SMILES: CN(C(=O)Oc1c(N2CCN(CCC3CNCCO3)C2=O)cc(C(F)(F)F)cc1C(F)(F)F)c1ccc(F)cc1 ; CN1CCOC(CCN2CCN(c3cc(C(F)(F)F)cc(C(F)(F)F)c3OC(=O)N(C)c3ccc(F)cc3)C2=O)C1 ; CN1CCOC(CCN2CCN(c3cc(C(F)(F)F)cc(C(F)(F)F)c3OC(=O)N(C)c3ccc(F)cc3)C2=O)C1 ; CN1CC(O)(CN2CCN(c3cc(C(F)(F)F)cc(C(F)(F)F)c3OC(=O)N(C)c3ccc(F)cc3)C2=O)C1]

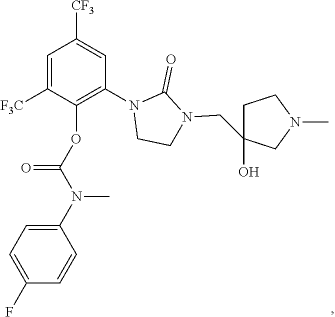 [SMILES: CN1CCC(O)(CN2CCN(c3cc(C(F)(F)F)cc(C(F)(F)F)c3OC(=O)N(C)c3ccc(F)cc3)C2=O)C1]

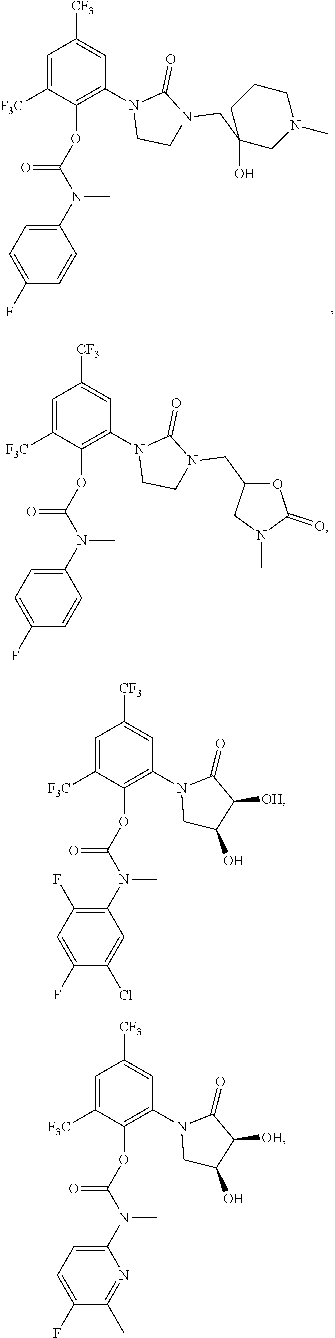

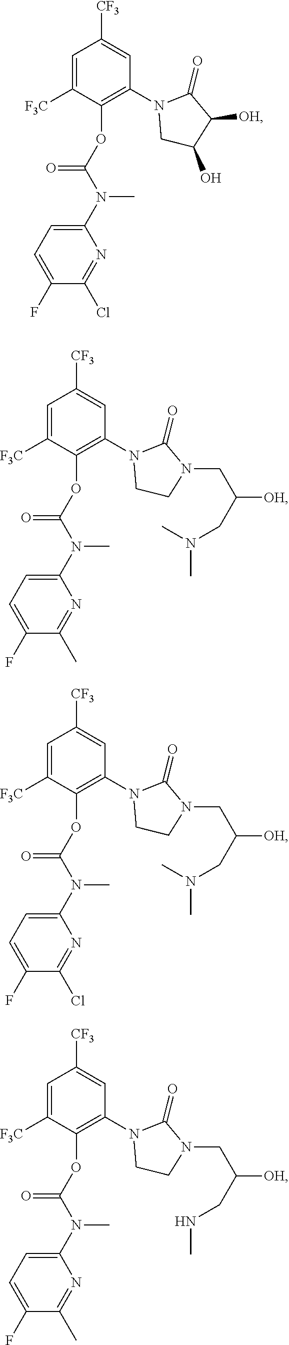

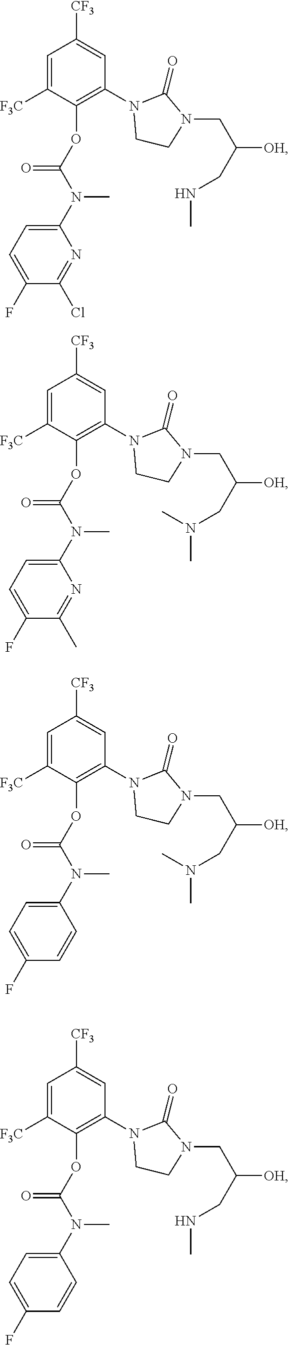

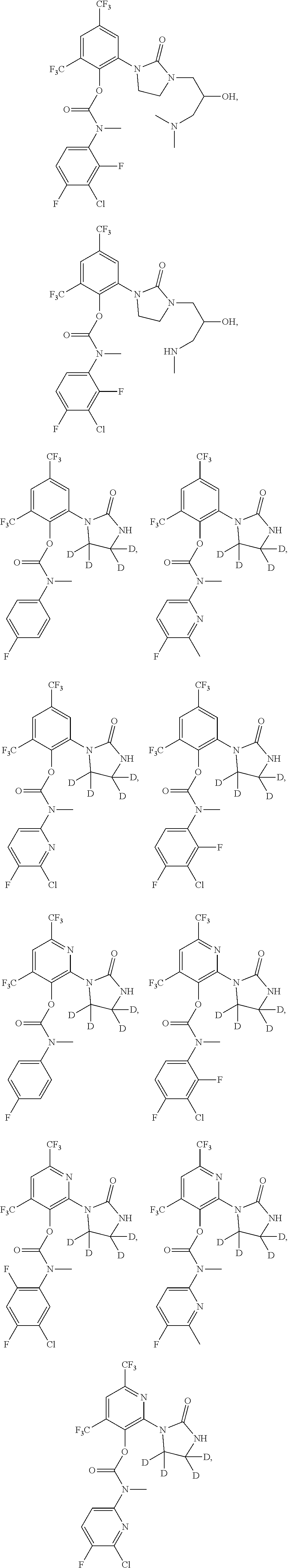

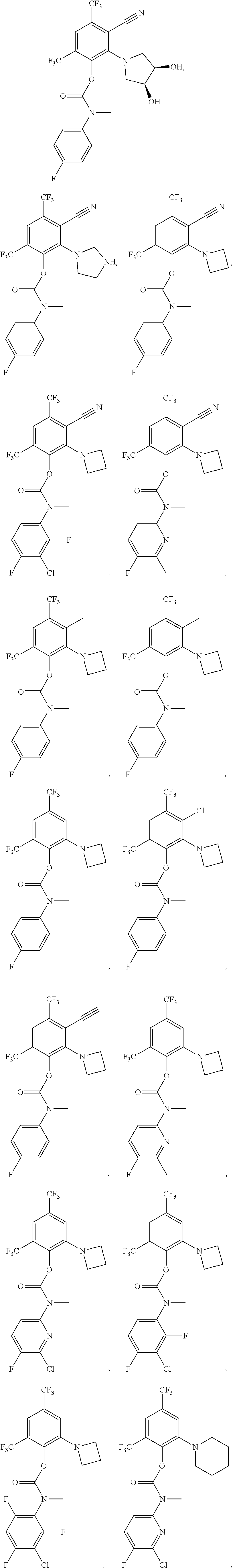

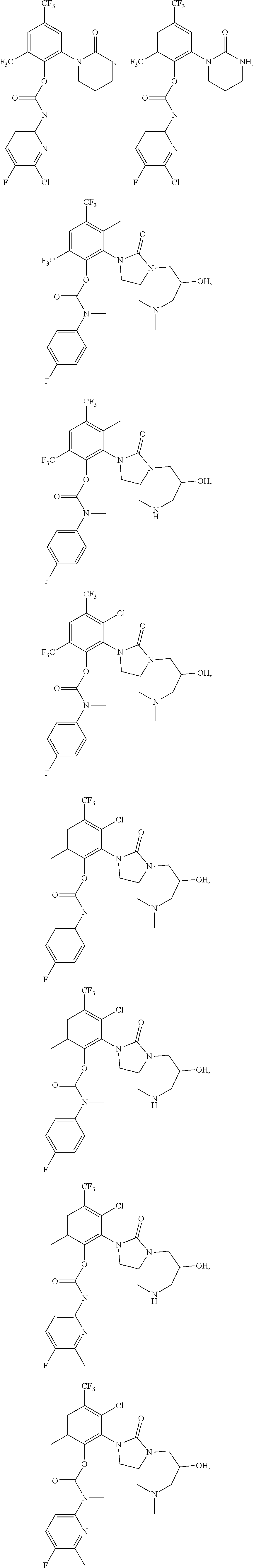

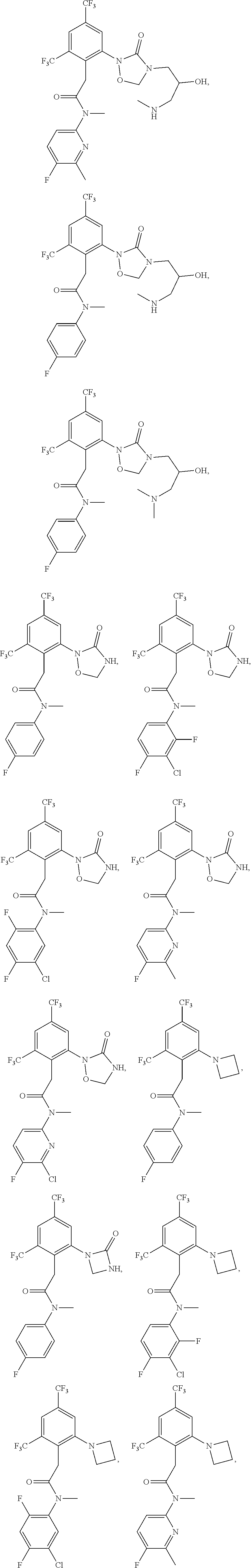

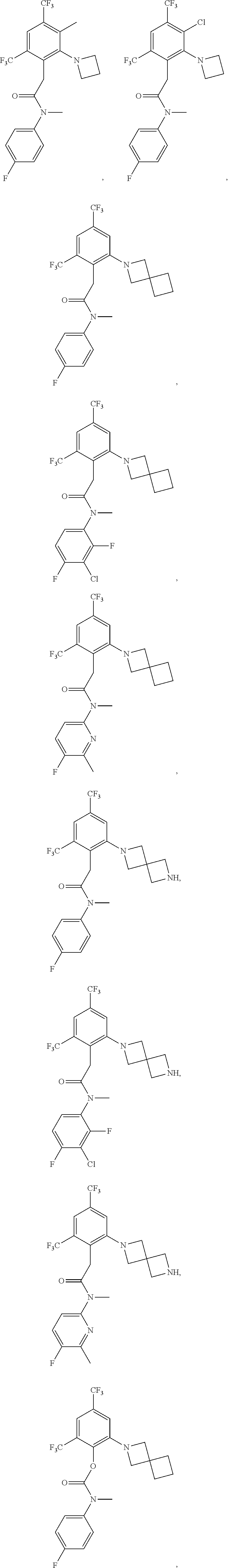

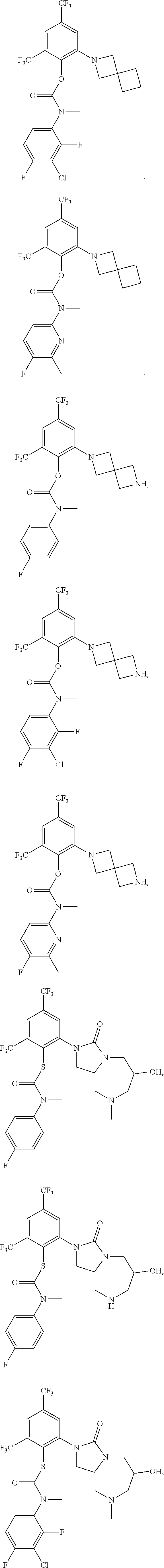

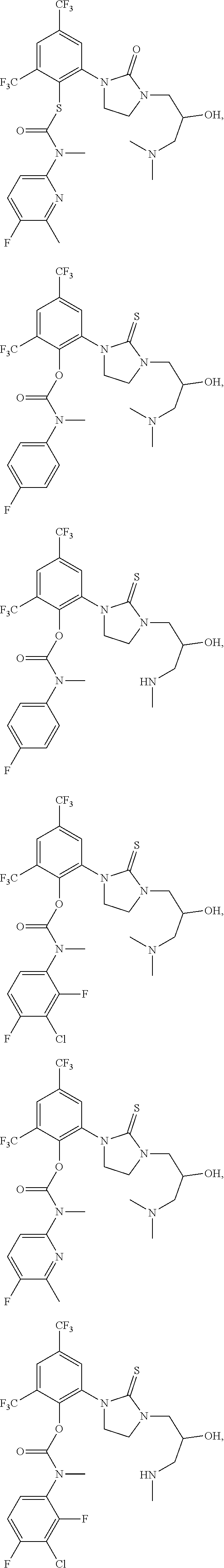

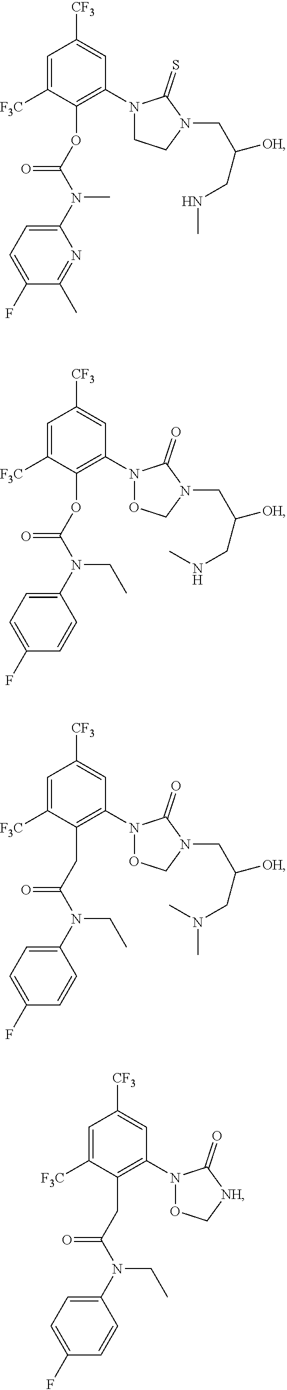

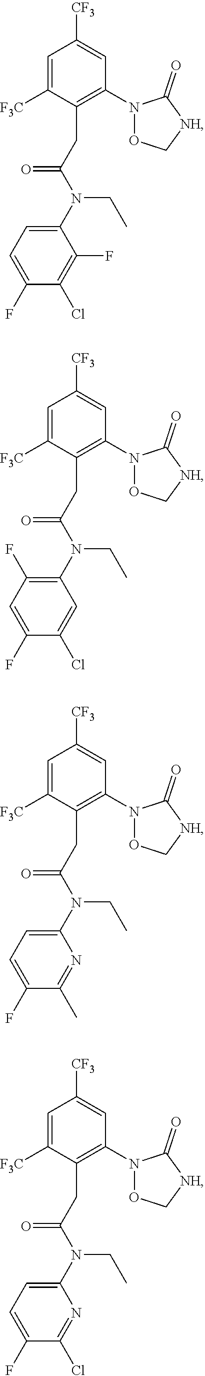

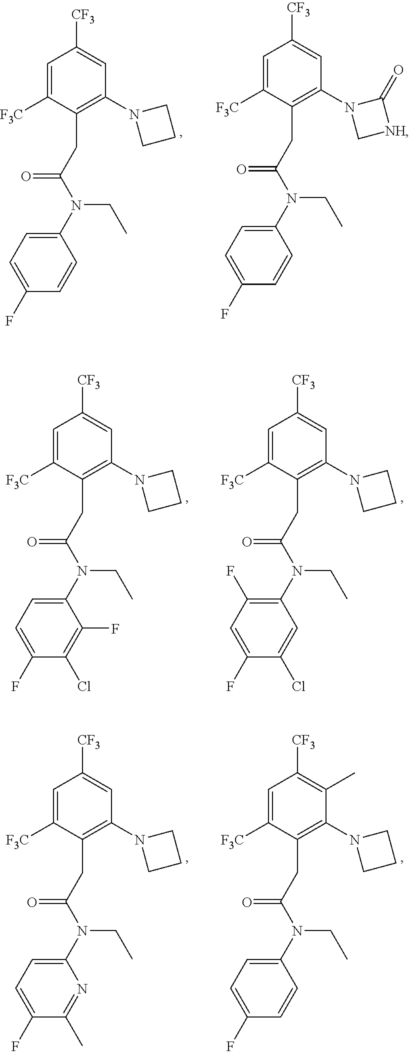

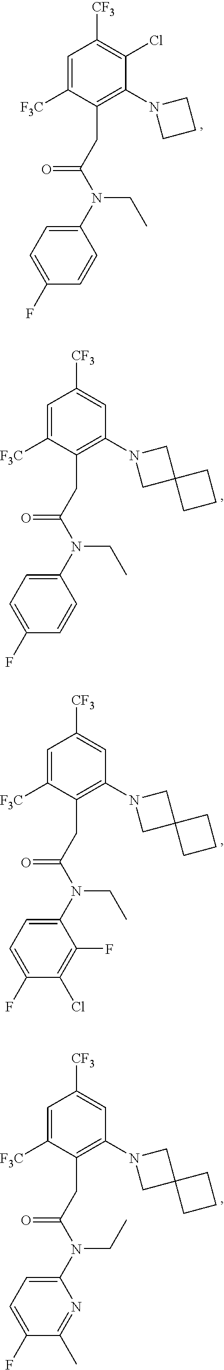

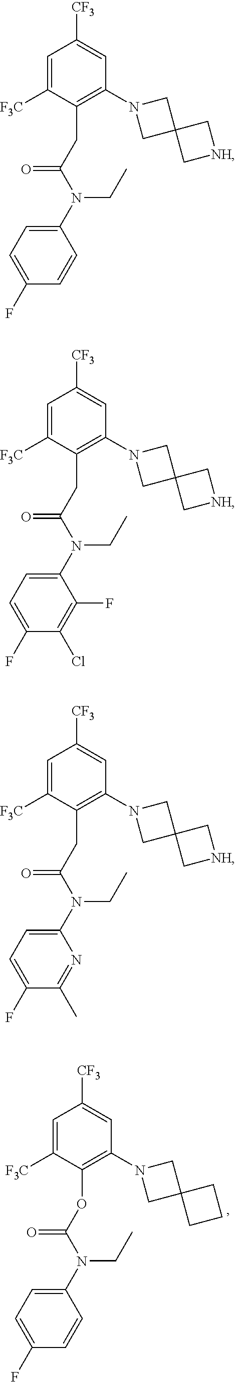

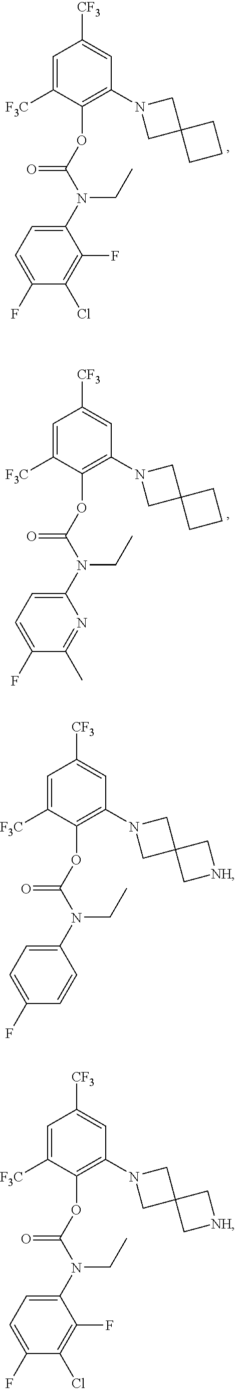

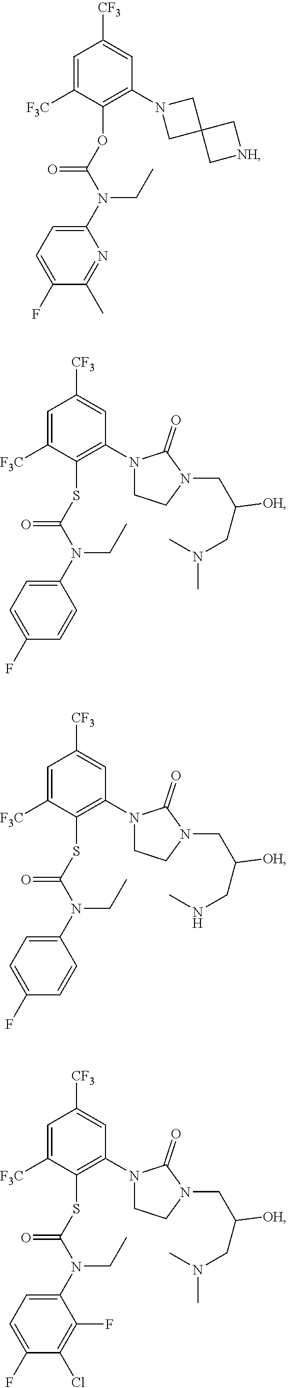

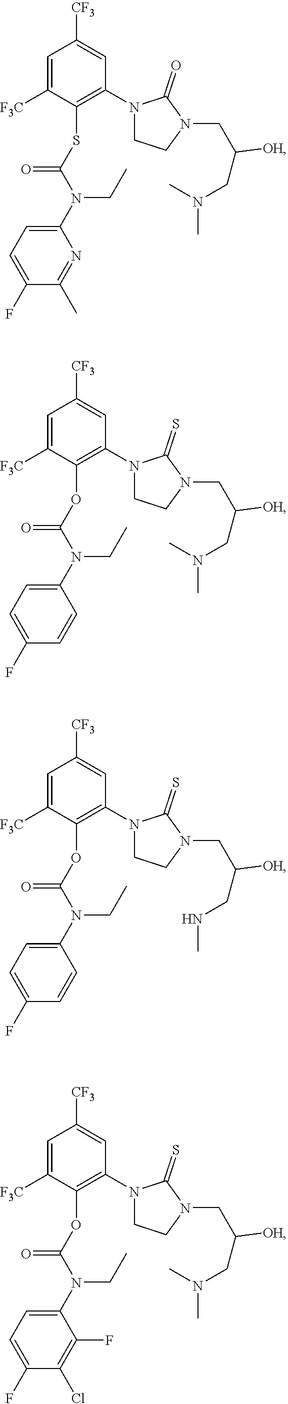 [SMILES: CCN(C(=O)Sc1c(N2CCN(CC(O)CN(C)C)C2=O)cc(C(F)(F)F)cc1C(F)(F)F)c1ccc(F)c(C)n1 ; CCN(C(=O)Oc1c(N2CCN(CC(O)CN(C)C)C2=S)cc(C(F)(F)F)cc1C(F)(F)F)c1ccc(F)cc1 ; CCN(C(=O)Oc1c(N2CCN(CC(O)CNC)C2=S)cc(C(F)(F)F)cc1C(F)(F)F)c1ccc(F)cc1 ; CCN(C(=O)Oc1c(N2CCN(CC(O)CN(C)C)C2=S)cc(C(F)(F)F)cc1C(F)(F)F)c1ccc(F)c(Cl)c1F]

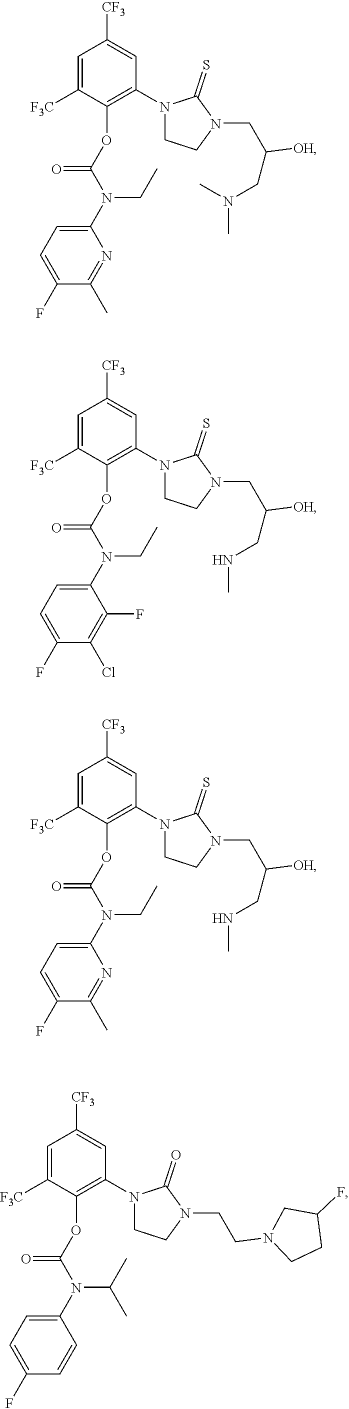 [SMILES: CCN(C(=O)Oc1c(N2CCN(CC(O)CN(C)C)C2=S)cc(C(F)(F)F)cc1C(F)(F)F)c1ccc(F)c(C)n1 ; CCN(C(=O)Oc1c(N2CCN(CC(O)CNC)C2=S)cc(C(F)(F)F)cc1C(F)(F)F)c1ccc(F)c(Cl)c1F ; CCN(C(=O)Oc1c(N2CCN(CC(O)CNC)C2=S)cc(C(F)(F)F)cc1C(F)(F)F)c1ccc(F)c(C)n1 ; CC(C)N(C(=O)Oc1c(N2CCN(CCN3CCC(F)C3)C2=O)cc(C(F)(F)F)cc1C(F)(F)F)c1ccc(F)cc1]

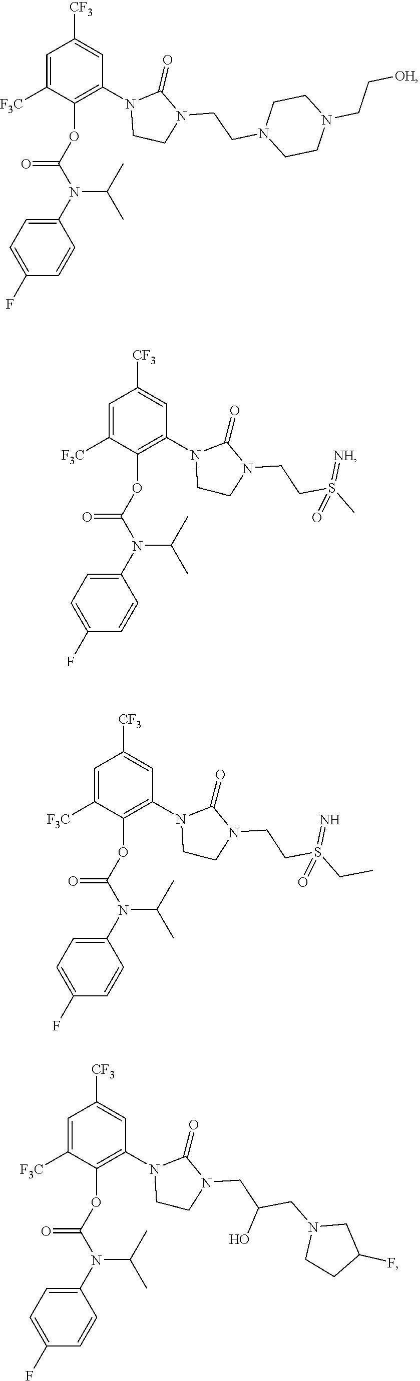 [SMILES: CC(C)N(C(=O)Oc1c(N2CCN(CCN3CCN(CCO)CC3)C2=O)cc(C(F)(F)F)cc1C(F)(F)F)c1ccc(F)cc1 ; CC(C)N(C(=O)Oc1c(N2CCN(CCS(C)(=N)=O)C2=O)cc(C(F)(F)F)cc1C(F)(F)F)c1ccc(F)cc1 ; CCS(=N)(=O)CCN1CCN(c2cc(C(F)(F)F)cc(C(F)(F)F)c2OC(=O)N(c2ccc(F)cc2)C(C)C)C1=O ; CC(C)N(C(=O)Oc1c(N2CCN(CC(O)CN3CCC(F)C3)C2=O)cc(C(F)(F)F)cc1C(F)(F)F)c1ccc(F)cc1]

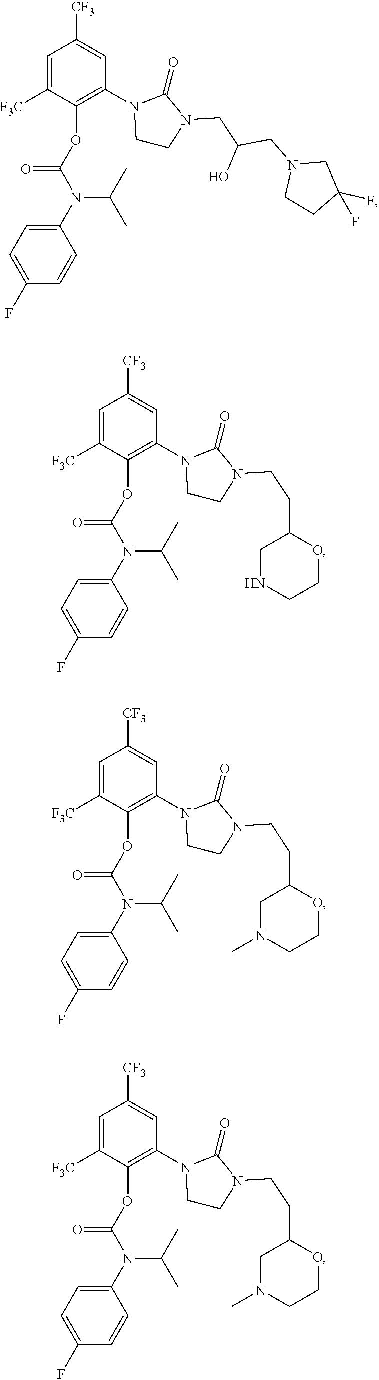 [SMILES: CC(C)N(C(=O)Oc1c(N2CCN(CC(O)CN3CCC(F)(F)C3)C2=O)cc(C(F)(F)F)cc1C(F)(F)F)c1ccc(F)cc1 ; CC(C)N(C(=O)Oc1c(N2CCN(CCC3CNCCO3)C2=O)cc(C(F)(F)F)cc1C(F)(F)F)c1ccc(F)cc1 ; CC(C)N(C(=O)Oc1c(N2CCN(CCC3CN(C)CCO3)C2=O)cc(C(F)(F)F)cc1C(F)(F)F)c1ccc(F)cc1 ; CC(C)N(C(=O)Oc1c(N2CCN(CCC3CN(C)CCO3)C2=O)cc(C(F)(F)F)cc1C(F)(F)F)c1ccc(F)cc1]

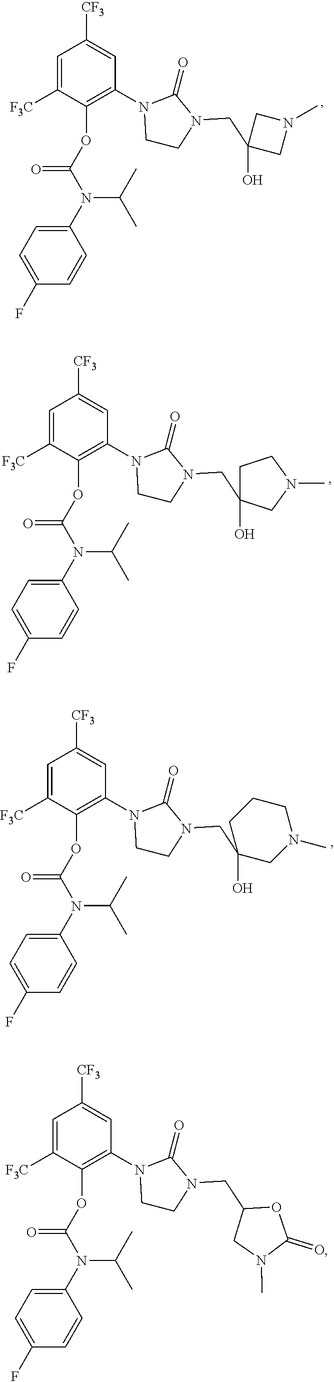

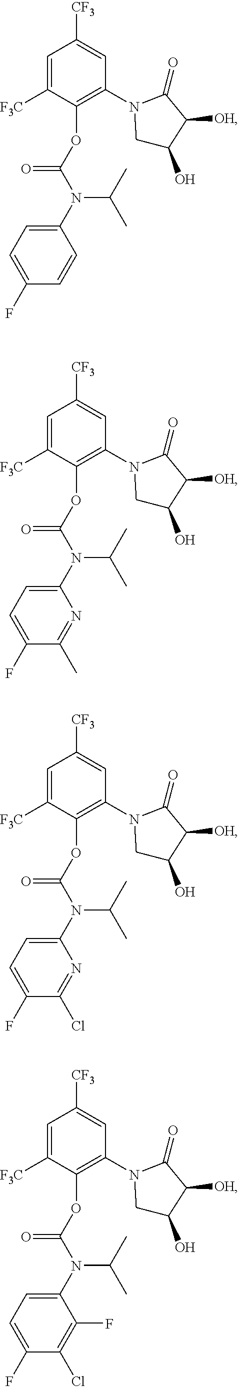

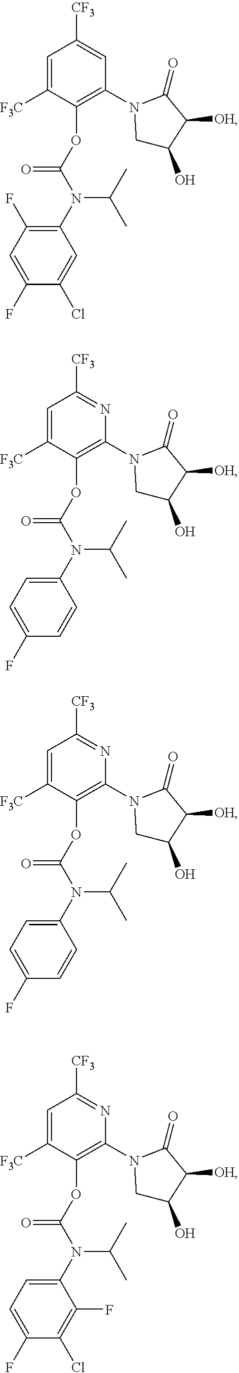

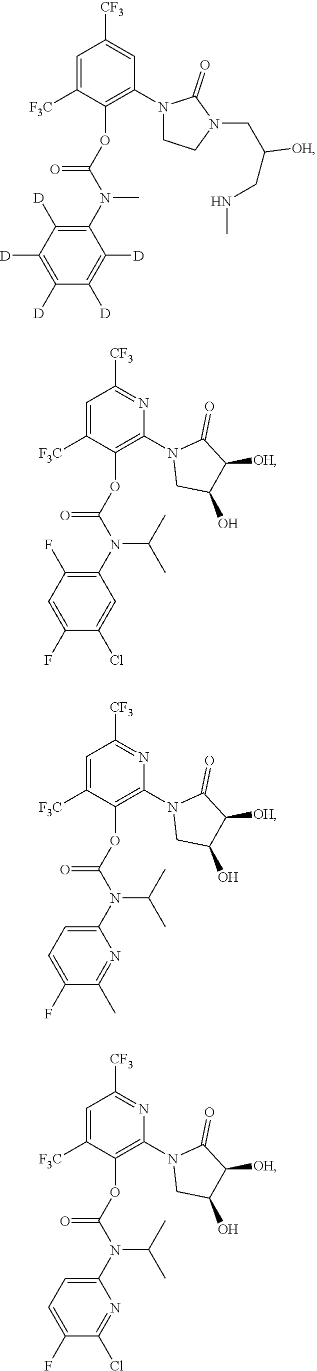

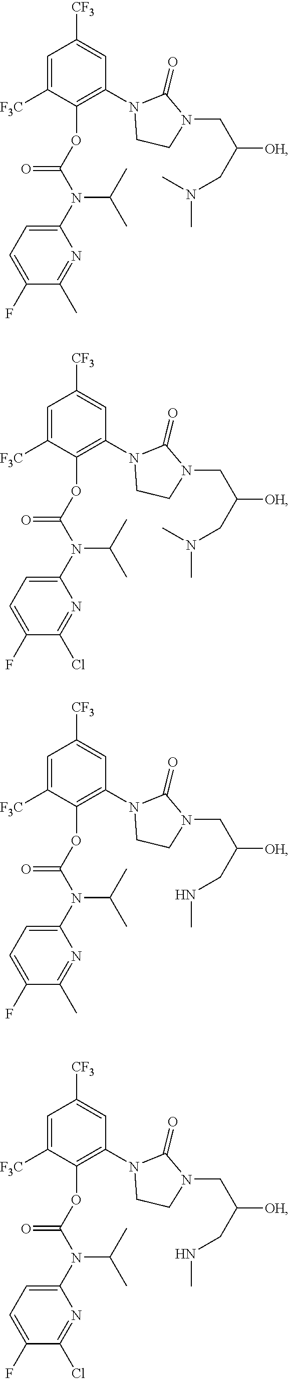

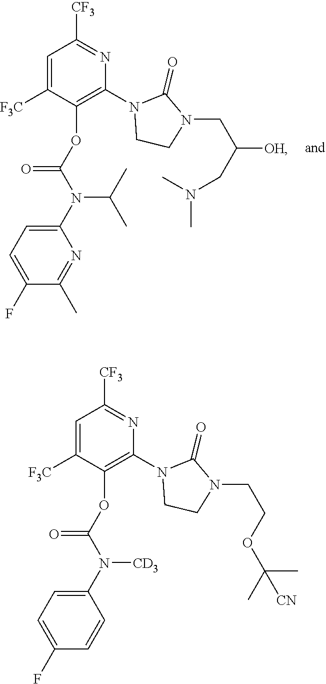

### Salts

Certain compounds of the Formula (I) can exist in the form of salts, for example acid addition salts or, in certain cases salts of organic and inorganic bases such as carboxylate, sulfonate and phosphate salts. The term “salts” embraces addition salts of free acids or free bases which are compounds of the invention. The term “pharmaceutically acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of this invention.

All such salts are within the scope of this invention, and references to compounds of the Formula (I) include the salt forms of the compounds. The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, non-aqueous media such as ether, ethyl acetate, ethanol, isopropanol, or acetonitrile are used.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), a-oxoglutaric, glycolic, hippuric, hydrohalic acids (e.g. hydrobromic, hydrochloric, hydriodic), isethionic, lactic (e.g. (+)-Llactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, Lpyroglutamic, salicylic, 4-amino-salicylic, sebacic, stearic, succinic, sulfuric, tannic, (+)-Ltartaric, thiocyanic, p-toluenesulfonic, undecylenic and valeric acids, as well as acylated amino acids and cation exchange resins. One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, methanesulfonic (mesylate), ethanesulfonic, naphthalenesulfonic, valeric, acetic, propanoic, butanoic, malonic, glucuronic and lactobionic acids. One particular salt is the hydrochloride salt. Where the compounds of the formula (I) contain an amine function, these may form quaternary ammonium salts, for example by reaction with an alkylating agent according to methods well known to the skilled person. Such quaternary ammonium compounds are within the scope of formula (I). The compounds of the invention may exist as mono- or di-salts depending upon the pKa of the acid from which the salt is formed. It will be appreciated that for use in medicine the salts of the compounds of formula (I) should be pharmaceutically acceptable. Suitable pharmaceutically acceptable salts will be apparent to those skilled in the art.

Pharmaceutically acceptable salts include those described by Berge, Bighley and Monkhouse, J. Pharm. Sci. 1977, 66, pp. 1-19. Suitable pharmaceutically acceptable acid addition salts may be prepared from an inorganic acid or from an organic acid. Examples of inorganic acids include hydrochloric, hydrobromic, hydroiodic, nitric, carbonic, sulfuric and phosphoric acids. Appropriate organic acids may be selected from aliphatic, cycloaliphatic, aromatic, araliphatic, heterocyclic, carboxylic and sulfonic classes of organic acids, examples of which include formic, acetic, pivalic, propionic, furoic, mucic, isethionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, -hydroxybutyric, salicylic, galactaric, camphorosulfonic and galacturonic acid. Examples of pharmaceutically unacceptable acid addition salts include, for example, perchlorates and tetrafluoroborates. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention. Certain of the compounds of formula (I) may form acid addition salts with one or more equivalents of the acid. The present invention includes within its scope all possible stoichiometric and non-stoichiometric forms.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chlooprocaine, choline, diethanolamine, ethylenediamine, tromethamine, meglumine (N-methylglucamine) and procaine. Examples of pharmaceutically unacceptable base addition salts include lithium salts and cyanate salts.

All of these salts may be prepared by conventional means from the corresponding compound according to Formula I by reacting, for example, the appropriate acid or base with the compound according to Formula I. Preferably the salts are in crystalline form, and preferably prepared by crystallization of the salt from a suitable solvent. The person skilled in the art will know how to prepare and select suitable salt forms for example, as described in Handbook of Pharmaceutical Salts: Properties, Selectin and Use by P. H. Stahl and C. G. Wermuth (Wiley-VCH 2002).

### Solvates

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Pharmaceutically acceptable solvates of the compound of the invention are within the scope of the invention. In one embodiment, the pharmaceutically acceptable solvates of the compounds of the invention include the hydrate thereof. In one embodiment, said crystalline form of the compounds of formula (I) is a cocrystal or coformer. Such a cocrystal or coformer may be prepared using water-soluble molecules such as saccharin, caffeine, nicotinamide or carboxylic acids. Coformers may be prepared as described in Emami S et al (2018) Biolmpacts 8(4), 305-320, the techniques of which are herein incorporated by reference. It will be understood that the invention includes pharmaceutically acceptable derivatives of compounds of formula (I) and that these are included within the scope of the invention. As used herein “pharmaceutically acceptable derivative” includes any pharmaceutically acceptable ester or salt of such ester of a compound of formula (I) which, upon administration to the recipient is capable of providing (directly or indirectly) a compound of formula (I) or an active metabolite or residue thereof.

### N-Oxides

Compounds of the formula (I) containing an amine function may also form N-oxides. A reference herein to a compound of the formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience. More particularly, N-oxides can be made by the procedure of L. W. Deady (Syn. Commun. 1977, 7, 509-514) in which the amine compound is reacted with m-chloroperoxybenzoic acid (mCPBA), for example, in an inert solvent such as dichloromethane.

### Prodrugs

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. All such prodrugs of compounds of the invention are included within the scope of the invention. Examples of pro-drug functionality suitable for the compounds of the present invention are described in Drugs of Today, 19, 9, 1983, 499-538 and in Topics in Chemistry, Chapter 31, pp. 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Also included within the scope of the compound and various salts of the invention are polymorphs thereof.

### Enantiomers

Where chiral centres are present in compounds of formula (I), the present invention includes within its scope all possible enantiomers and diastereoisomers, including mixtures thereof. The different isomeric forms may be separated or resolved one from the other by conventional methods, or any given isomer may be obtained by conventional synthetic methods or by stereospecific or asymmetric syntheses. The invention also extends to any tautomeric forms or mixtures thereof.

### Isotopes

The subject invention also includes all pharmaceutically acceptable isotopically-labelled compounds which are identical to those recited in formula (I) but for the fact that one or more atoms are replaced by an atom having an atomic mass or mass number different from the atomic mass or mass number most commonly found in nature. Examples of isotopes suitable for inclusion in the compounds of the invention comprise isotopes of hydrogen, such as ²H (D) and ³H (T), carbon, such as ¹¹C, ¹³C, and ¹⁴C, chlorine, such as ³⁶Cl, fluorine, such as ¹⁸F, iodine, such as ¹²³I, ¹²⁵I, and ¹³¹I, nitrogen, such as ¹³N and ¹⁵N, oxygen, such as ¹⁵O, ¹⁷, and ¹⁸O, phosphorus, such as ³²P, and sulfur, such as ³⁵S. Certain isotopically-labelled compounds of formula (I), for example, those incorporating a radioactive isotope, are useful in drug and/or substrate tissue distribution studies. The compounds of formula (I) can also have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase) etc. The radioactive isotopes tritium, i.e. ³H (T), and carbon-14, i.e. ¹⁴C, are particularly useful for this purpose in view of their ease of incorporation and ready means of detection. Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O, and ¹³N, can be useful in Positron Emission Topography (PET) studies for examining target occupancy.

Isotopically-labelled compounds of formula (I) can generally be prepared by conventional techniques known to those skilled in the art or by processes analogous to those described in the accompanying Examples and Preparations using appropriate isotopically-labelled reagents in place of the non-labelled reagent previously employed.

### Purity

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are given on a weight for weight basis). Impure preparations of the compounds may be used for preparing the more pure forms used in the pharmaceutical compositions.

### Processes

According to a further aspect of the present invention there is provided a process for the preparation of compounds of formula (I) and derivatives thereof. The following schemes are examples of synthetic schemes that may be used to synthesise the compounds of the invention. In the following schemes reactive groups can be protected with protecting groups and de-protected according to well established techniques.

Compounds of Formula I may be prepared according to the general methods of Schemes 4-5. Certain compounds of Formula I, identified as having the structure of Formula Ia, may be prepared using the general method shown in Scheme 4. Other compounds of Formula I, identified as having the structure of Formula Ib, may be prepared using the general method shown in Scheme 5. It may be appreciated that the compounds of formula Ia are compounds of Formula I wherein U is O while compounds of Formula Ib are compounds of Formula I wherein U is CH₂. Intermediates 2, 5 and X may be prepared using the general methods shown in Schemes 1-3.

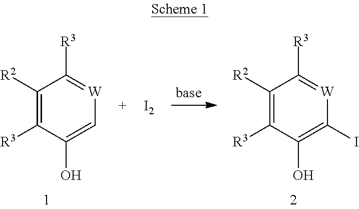

Compounds of Formula 2 may be prepared as shown in Scheme 1. Compound 1, a known compound or a compound prepared according to known methods, is treated with iodine in the presence of a suitable base such as pyridine, N,N-dimethylpyridine, triethylamine, diisopropylethylamine, diazabicyclononene, diazabicycloundecene, sodium hydroxide, potassium hydroxide, sodium carbonate, potassium carbonate, sodium hydride, potassium hydride, sodium tert-butoxide, potassium tert-butoxide, N-butyllithium, lithium diisopropylamide and the like, optionally in the presence of a suitable solvent such as water, hexane, heptane, cyclohexane, dichloromethane, chloroform, carbon tetrachloride, ethylacetate, tetrahydrofuran, benzene, toluene, diethylether, methanol, ethanol, N-methylpyrrolidone, dimethylsulfoxide, dimethylformamide and the like, optionally in the presence of a phase transfer agent such as tetrabutylammonium bromide, benzyltriethylammonium chloride, methyltribenzylammonium chloride, hexadecyltributylphosphonium bromide, 18-Crown-6 and the like, optionally with heating, optionally with microwave irradiation, to provide a compound of Formula 2.

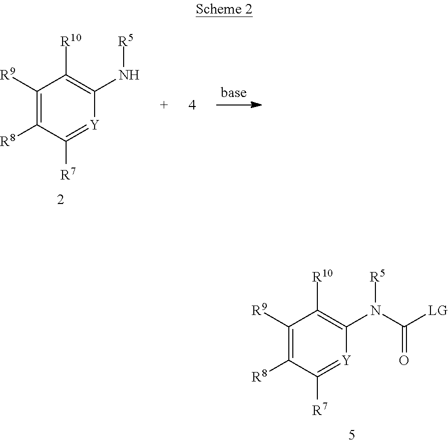

Compounds of Formula 5 may be prepared as shown in Scheme 2. Compound 3, a known compound or a compound prepared using known methods, is treated with a carbamoylation agent 4 such as phosgene, diphosgene, triphosgene, carbonyldiimidazole, disuccinimidyl carbonate and the like where LG is a suitable leaving group such as halogen, imidazole, succinimide and the like, in the presence of a suitable base such as pyridine, N,N-dimethylpyridine, triethylamine, diisopropylethylamine, diazabicyclononene, diazabicycloundecene, sodium hydroxide, potassium hydroxide, sodium carbonate, potassium carbonate, sodium hydride, potassium hydride, sodium tert-butoxide, potassium tert-butoxide, N-butyllithium, lithium diisopropylamide and the like, optionally in the presence of a suitable solvent such as water, hexane, heptane, cyclohexane, dichloromethane, chloroform, carbon tetrachloride, ethylacetate, tetrahydrofuran, benzene, toluene, diethylether, methanol, ethanol, N-methylpyrrolidone, dimethylsulfoxide, dimethylformamide and the like, optionally in the presence of a phase transfer agent such as tetrabutylammonium bromide, benzyltriethylammonium chloride, methyltribenzylammonium chloride, hexadecyltributylphosphonium bromide, 18-Crown-6 and the like, optionally with heating, optionally with microwave irradiation to provide a compound of Formula 5.

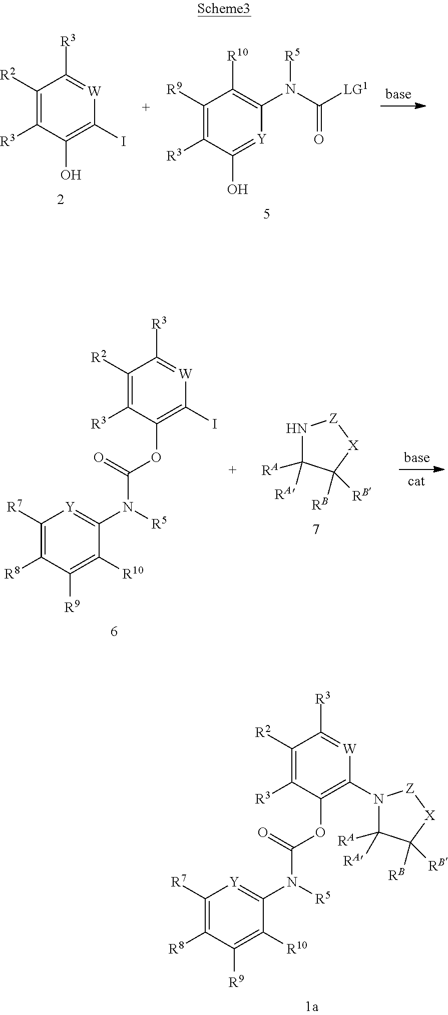

Some of the compounds of Formula I may be prepared as shown in Scheme 3. According to Scheme 3, a compound of Formula 2 is reacted with a compound of Formula 5 in the presence of in the presence of a suitable base such as pyridine, N,N-dimethylpyridine, triethylamine, diisopropylethylamine, diazabicyclononene, diazabicycloundecene, sodium hydroxide, potassium hydroxide, sodium carbonate, potassium carbonate, sodium hydride, potassium hydride, sodium tert-butoxide, potassium tert-butoxide, N-butyllithium, lithium diisopropylamide and the like, optionally in the presence of a suitable solvent such as water, hexane, heptane, cyclohexane, dichloromethane, chloroform, carbon tetrachloride, ethylacetate, tetrahydrofuran, benzene, toluene, diethylether, methanol, ethanol, N-methylpyrrolidone, dimethylsulfoxide, dimethylformamide and the like, optionally in the presence of a phase transfer agent such as tetrabutylammonium bromide, benzyltriethylammonium chloride, methyltribenzylammonium chloride, hexadecyltributylphosphonium bromide, 18-Crown-6 and the like, optionally with heating, optionally with microwave irradiation to provide a compound of Formula 6. A compound of Formula 6 is then reacted with a compound of Formula 7, a known compound or a compound prepared by known methods, in the presence of a suitable catalyst such as copper (I) bromide, copper (I) iodide, nickel bromide and the like, a suitable coordinating agent such as N,N-dimethylethylenediamine, a suitable base such as potassium carbonate, potassium phosphate, cesium carbonate, sodium tert-butoxide and the like, a suitable ligand such as cesium fluoride, a suitable solvent such as water, tetrahydrofuran, 1,4-dioxane, dimethylsulfoxide, dimethylformamide, acetonitrile, optionally in the presence of molecular sieves, optionally with heating, optionally with microwave irradiation, to provide a compound of Formula I.

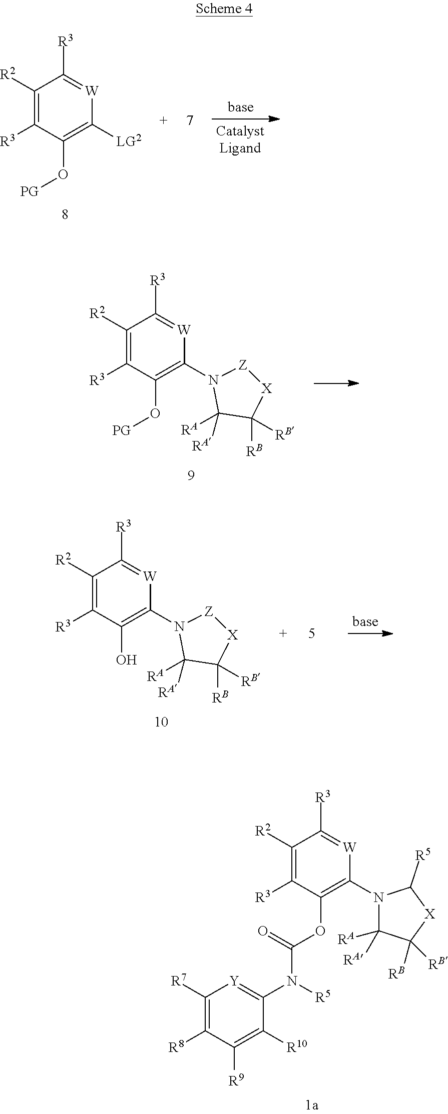

Alternatively, some of the compounds of formula I may be prepared as shown in Scheme 4. According to Scheme 4, a compound of Formula 8, a known compound or a compound prepared using known methods wherein PG represents a suitable protecting group such as benzyl, tert-butyldimethylsilyl, methoxymethyl, tetrohydropyranyl and the like and LG² represents a suitable leaving group such as chloro, bromo, iodo, mesyl, trifluoromesyl, p-toleuensulfonyl and the like, is treated with a compound of Formula 7, a known compound or a compound prepared using known methods, in the presence of a suitable base such as sodium tert-butoxide, lithium hexamethyldisylazide, cesium carbonate, potassium phosphate and the like, a suitable catalyst such as palladium (0) bis(dibenzylidineacetone), palladium acetate, (DPPF)PdCl₂ and the like, a suitable ligand such as 2,2′-Bis(diphenylphosphino)-1,1′-binaphthyl (BINAP), tri-tert-butylphosphine, Xantphos and the like, and a suitable solvent such as toluene, xylene, tetrahydrofuran, 1,4-dioxane, dimethoxyethane, butanol, tert-butanol and the like, optionally with heating, optionally with microwave irradiation, to provide a compound of Formula 9. A compound of Formula 9 is then reacted under suitable conditions to remove the protecting group such as catalytic hydrogenation in the presence of a suitable catalyst such as palladium on carbon, palladium hydroxide and the like, hydrogen fluoride/pyridine, diaminoethylsulfur trifluoride, aqueous acid and the like to provide a compound of Formula 10. A compound of Formula 10 is then reacted with a compound of Formula 5 in the presence of in the presence of a suitable base such as pyridine, N,N-dimethylpyridine, triethylamine, diisopropylethylamine, diazabicyclononene, diazabicycloundecene, sodium hydroxide, potassium hydroxide, sodium carbonate, potassium carbonate, sodium hydride, potassium hydride, sodium tert-butoxide, potassium tert-butoxide, N-butyllithium, lithium diisopropylamide and the like, optionally in the presence of a suitable solvent such as water, hexane, heptane, cyclohexane, dichloromethane, chloroform, carbon tetrachloride, ethylacetate, tetrahydrofuran, benzene, toluene, diethylether, methanol, ethanol, N-methylpyrrolidone, dimethylsulfoxide, dimethylformamide and the like, optionally in the presence of a phase transfer agent such as tetrabutylammonium bromide, benzyltriethylammonium chloride, methyltribenzylammonium chloride, hexadecyltributylphosphonium bromide, 18-Crown-6 and the like, optionally with heating, optionally with microwave irradiation to provide a compound of Formula I.

In the aforesaid processes, certain functional groups which would be sensitive to the reaction conditions may be protected by protecting groups. A protecting group is a derivative of a chemical functional group which would otherwise be incompatible with the conditions required to perform a particular reaction which, after the reaction has been carried out, can e removed to re-generate the original functional group, which is thereby considered to have been “protected”. Any chemical functionality that is a structural component of any of the reagents used to synthesize compounds of this invention may be optionally protected with a chemical protecting group if such a protection group is useful in the Synthesis of compounds of this invention. The person skilled in the art knows when protecting groups are indicated, how to select such groups, and processes that can be used for selectively introducing and selectively removing them, because methods of selecting and using protecting groups have been extensively documents in the chemical literature. Techniques for selecting, incorporating and removing chemical protecting groups may be found, for example, in Protective Groups in Organic Synthesis by Theodora W. Greene, Peter G. M. Wuts, John Wiley & Sons Ltd., the entire disclosure of which is incorporated herein by reference.

It will be appreciated by one skilled in the art that the processes described are not the exclusive means by which compounds of Formula I may be synthesized and that a repertoire of synthetic organic reactions is available to be potentially employed in synthesizing compounds of the invention. The person skilled in the art knows how to select and implement appropriate synthetic routes. Suitable synthetic methods may be identified by reference to the literature, including reference sources such as Comprehensive Organic Synthesis, Ed. B. M. Trost and I. Fleming (Pergamon Press, 1991), Comprehensive Organic Functional Group Transformations, Ed. A. R. Katritzky, O. Meth-Cohn, and C. W. Rees (Pergamon Press, 1996), Comprehensive Organic Functional Group Transformations II, Ed. A, R. Katritzky and R. J. K. Taylor (Editor) (Elsevier, 2^{nd} Edition, 2004), Comprehensive Heterocyclic Chemistry, Ed. A. R. Katritzky and C. W. Rees (Pergamon Press, 1984), and Comprehensive Heterocyclic Chemistry II, Ed. A. R. Katritzky, C. W. Rees and E. F. V. Scriven (Pergamon Press, 1996).

The compounds of Formula I and intermediates may be isolated from their reaction mixtures and purified by standard techniques such as filtration, liquid-liquid extraction, solid phase extraction, distillation, recrystallization or chromatography.

It will be understood that when compounds of Formula I the present invention contain one or more chiral centers, the compounds may exist in, and may be isolated s pure enantiomeric or diasteromeric forms or as racemic mixtures. The present invention therefore includes any possible enantiomers, diastereomers, racemates or mixtures thereof of the compounds of the invention which are biologically active in the treatment of cancer.

### Preparation of the Compounds of the Invention

Compounds of formula (I) may be prepared by the general schemes described herein, using the synthetic method known by those skilled in the art. The following examples illustrate non-limiting embodiments of the invention.

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

In one embodiment, the compounds of the invention may exist as tautomers. All tautomers are included within the scope of the compounds presented herein.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

In one embodiment, sites on, for example, the aromatic ring portion of compounds of the invention are susceptible to various metabolic reactions. Incorporation of appropriate substituents on the aromatic ring structures may reduce, minimize or eliminate this metabolic pathway. In one embodiment, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In one embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In one embodiment, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In one embodiment, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

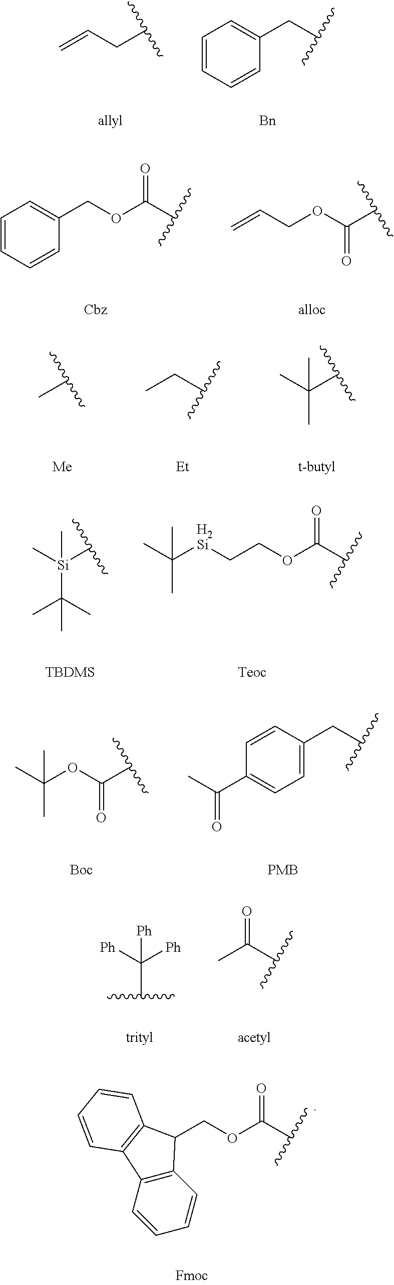

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Methods of the Invention

The invention includes a method of treating or preventing cancer in a subject in need thereof. The method comprises administering to the subject an effective amount of a therapeutic composition comprising a compound of the invention. Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the compositions of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers that can be treated with the compositions of the invention include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, that can be treated with the compositions of the invention, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In one embodiment, the cancer is selected from the group consisting of lung cancer, colon cancer, colorectal cancer, melanoma, breast cancer, ovarian cancer, prostate cancer, liver cancer, pancreatic cancer, CNS tumors (including brain tumors), neuroblastoma, leukemia, bone cancer, intestinal cancer, lymphoma, and combinations thereof. In one embodiment, the cancer is pancreatic cancer. In one embodiment, the cancer is prostate cancer. In one embodiment, the method further comprises administering to the subject an additional therapeutic agent. In one embodiment, the therapeutic agent is gemcitabine.

The invention also includes a method of treating or preventing pain or inflammation in a subject in need thereof. The method comprises administering to the subject an effective amount of a therapeutic composition comprising a compound of the invention. In one embodiment, the inflammation is selected from the group consisting of arthritic disorders, psoriasis, allergies, opioid tolerance, Crohn's Disease, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, and myocardial ischemia. In one embodiment, the arthritic disorder is selected from the group consisting of rheumatoid arthritis, spondyloarthropathies, gouty arthritis, osteoarthritis, systemic lupus erythematosus and juvenile arthritis. In one embodiment, the method further comprises administering to the subject an additional therapeutic agent.

In one embodiment, the pain is selected from the group consisting of pain resulting from cancer, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures. In one embodiment, the method further comprises administering to the subject an additional therapeutic agent.

The invention also includes a method of treating or preventing a disease or disorder associated with the NF-κB pathway in a subject in need thereof. The method comprises administering to the subject an effective amount of a therapeutic composition comprising a compound of the invention. Non-limiting examples of diseases or disorder associated with reactive oxygen species include ischemic diseases, inflammatory diseases, autoimmune diseases, cancer metastasis and invasion, and cachexia.

The invention also includes a method of treating or preventing a disease or disorder associated with reactive oxygen species (ROS) in a subject in need thereof. The method comprises administering to the subject an effective amount of a therapeutic composition comprising a compound of the invention. Non-limiting examples of diseases or disorder associated with reactive oxygen species include arteriosclerosis, myocardial infarction, diabetes, and cancer.

In one embodiment, administering the compound of the invention to the subject allows for administering a lower dose of the therapeutic agent compared to the dose of the therapeutic agent alone that is required to achieve similar results in treating or preventing cancer in the subject. For example, in one embodiment, the compound of the invention enhances the anti-cancer activity of the additional therapeutic compound, thereby allowing for a lower dose of the therapeutic compound to provide the same effect. In another embodiment, administering the compound of the invention to the subject allows for administering a lower dose of the therapeutic agent compared to the dose of the therapeutic agent alone that is required to achieve similar results in treating or preventing pain or inflammation in the subject.

In one embodiment, the compound of the invention and the therapeutic agent are co-administered to the subject. In another embodiment, the compound of the invention and the therapeutic agent are co-formulated and co-administered to the subject.

In one embodiment, the subject is a mammal. In another embodiment, the mammal is a human.

### Combination Therapies

The compounds of the present invention are intended to be useful in combination with one or more additional compounds. In certain embodiments, these additional compounds may comprise compounds of the present invention or therapeutic agents known to treat or reduce the symptoms or effects of cancer. Such compounds include, but are not limited to, chemotherapeutics and the like. In other embodiments, these additional compounds may comprise therapeutic agents known to treat or reduce the symptoms or effects of pain or inflammation.

In one embodiment, the compound of Formula (I) is used in combination with any known FDA-approved cancer drug. In one embodiment, the invention provides a method to treat cancer comprising treating the subject prior to, concurrently with, or subsequently to the administration of a compound disclosed herein, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diaminedichloroplatinum, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene). Additional examples of specific antiproliferative agents include, but are not limited to levamisole, gallium nitrate, granisetron, sargramostim strontium-89 chloride, filgrastim, pilocarpine, dexrazoxane, and ondansetron.

The compound of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; albumin-bound paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; imilimumab; mirtazapine; BrUOG 278; BrUOG 292; RAD0001; CT-011; folfirinox; tipifarnib; R115777; LDE225; calcitriol; AZD6244; AMG 655; AMG 479; BKM120; mFOLFOX6; NC-6004; cetuximab; IM-C225; LGX818; MEK162; BBI608; MEDI4736; vemurafenib; ipilimumab; ivolumab; nivolumab; panobinostat; leflunomide; CEP-32496; alemtuzumab; bevacizumab; ofatumumab; panitumumab; pembrolizumab; rituximab; trastuzumab; STAT3 inhibitors (e.g., STA-21, LLL-3, LLL12, XZH-5, S31-201, SF-1066, SF-1087, STX-0119, cryptotanshinone, curcumin, diferuloylmethane, FLLL11, FLLL12, FLLL32, FLLL62, C3, C30, C188, C188-9, LY5, OPB-31121, pyrimethamine, OPB-51602, AZD9150, etc.); hypoxia inducing factor 1 (HIF-1) inhibitors (e.g., LW6, digoxin, laurenditerpenol, PX-478, RX-0047, vitexin, KC7F2, YC-1, etc.) zinostatin stimalamer, Lynparza (olaparib), talazoparib, niraparib, and rucaparib.

In non-limiting examples, the compounds of the invention may be used in combination with one or more therapeutic agents (or a salt, solvate or prodrug thereof).

In certain embodiments, the compound of the invention may be administered to a subject in conjunction with (e.g. before, simultaneously, or following) any number of relevant treatment modalities including chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the compounds of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the compounds of the present invention are administered following B-cell ablative therapy such as agents that react with CD20, e.g., Rituxan. In another embodiment, the compounds of the present invention are administered in conjunction with Ospemifene, Tamoxifen, Raloxifene, or other drugs such as ICI 182,780 and RU 58668. Tamoxifen and Raloxifene may act as partial antiestrogens, and the drugs such as ICI 182,780 and RU 58668 may act as full antiestrogens. In another embodiment, the compounds of the invention are administered in conjunction with aromatase inhibitors. Non-limiting examples of aromatase inhibitors include Exemestane, Letrozole, and Anastrozole. In one embodiment, the therapeutic agent is gemcitabine.

In certain embodiments, the compounds of the invention may be administered to a subject in conjunction with (e.g. before, simultaneously, or following) an anti-inflammatory agent selected from the group consisting of nonsteroidal agents (“NSAIDS”) such as salicylates (e.g., salsalate, mesalamine, diflunisal, choline magnesium trisalicylate), diclofenac, diflunisal, etodolac, fenoprofen, flurbiprofen, ibuprofen, indomethacin, mefenamic acid, nabumetone, naproxen, piroxicam, phenyl butazone, ketoprofen, S-ketoprofen, ketorolac tromethamine, sulindac, tolmetin). Other anti-inflammatory drugs include steroidal agents such as beclomethasone, betamethasone, cortisone, dexamethasone, fluocinolone, flunisolide, fluticasone proprionate, fluorinated-corticoids, triamcinolone-diacetate, hydorcortisone, prednisolone, methylprednisolone, and prednisone. Immunosuppressive agents (e.g., adenocorticosteroids, cyclosporin), antihistamines and decongestants (e.g., astemizole(histamine I II -receptor antagonist), azatidine, brompheniramine, clemastine, chlogheniramine, cromolyn, cyproheptadine, diphenylimidazole, diphenhydramine hydrochloride, hydroxyzine, glycyrrhetic acid, homochlorocyclizine hydrochloride, ketotifen, loratadine, naphazoline, phenindamine, pheniramine, promethazine, terfenadine, trimeprazine, tripelennamine, tranilast, and the decongestants phenylpropanolamine and pseudoephedrine. In one embodiment, the therapeutic agent is a nonsteroidal anti-inflammatory drug (NSAID), as would be understood by one of ordinary skill in the art.

A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In certain embodiments, the compounds of Formula (I) may be administered to a subject in conjunction with cancer drugs that target DNA repair factors (i.e. PARP1, PARG, ATM, ATR, DNApk, RAD51, CHK1, WEE1, topoisomerase I, topoisomerase II) and/or act as genotoxic agents (i.e. chemotherapies and radiation/radiotherapy, proton therapy) and induce DNA damage. In some embodiments, the cancer drugs that target DNA repair factors are one or more selected from the group consisting of DNA damage agents, platinum agents, DNA damage response inhibitors, ATR inhibitors, WEE1 inhibitors, NDA-PK inhibitors, and ATM inhibitors. Compounds of Formula I may be especially useful as radiosensitizers or chemosensitizers, and act synergistically with PARP inhibitors (i.e. olaparib, niraparib, talazoparib, rucaparib) and topoisomerase inhibitors (etoposide, camptothecin, toptecan, doxorubicin, daunorubicin) and ATR inhibitors and DNApk inhibitors.

### Administration/Dosage/Formulations

The regimen of administration may affect what constitutes an effective amount. The therapeutic formulations may be administered to the subject either before or after the onset of cancer. Further, several divided dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, (e.g., human), may be carried out using known procedures, at dosages and for periods of time effective to treat cancer in the patient. An effective amount of the therapeutic compound necessary to achieve a therapeutic effect may vary according to factors such as the state of the disease or disorder in the patient; the age, sex, and weight of the patient; and the ability of the therapeutic compound to treat a cancer in the patient. Dosage regimens may be adjusted to provide the optimum therapeutic response. For example, several divided doses may be administered daily. In another example, the dose may be proportionally reduced as indicated by the exigencies of the therapeutic situation. A non-limiting example of an effective dose range for a therapeutic compound of the invention is from about 1 mg/kg to about 5,000 mg/kg of body weight/per day. One of ordinary skill in the art would be able to assess the relevant factors and make the determination regarding the effective amount of the therapeutic compound without undue experimentation.

Actual dosage levels of the active ingredients in the pharmaceutical compositions of this invention may be varied to obtain an amount of the active ingredient that is effective to achieve the desired therapeutic response for a particular patient, composition, and mode of administration, without generating excessive side effects in the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical professional, e.g., physician or veterinarian, having ordinary skill in the art may readily determine and prescribe the effective amount of the pharmaceutical composition required. For example, the physician or veterinarian could start with a dosage of the compound of the invention in the pharmaceutical composition at a level that is lower than the level required to achieve the desired therapeutic effect, and then increase the dosage over time until the desired effect is achieved.

In particular embodiments, it is advantageous to formulate the compound in dosage unit form for ease of administration and uniformity of dosage. “Dosage unit form” as used herein refers to a physically discrete unit containing a predetermined quantity of therapeutic compound calculated to produce the desired therapeutic effect, in association with the required pharmaceutical vehicle. The dosage unit forms of the invention can be selected based upon (a) the unique characteristics of the therapeutic compound and the particular therapeutic effect to be achieved, and (b) the limitations inherent in the art of compounding/formulating such a therapeutic compound for the treatment of cancer in a patient.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), vegetable oils, and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments, it is useful to include isotonic agents, for example, sugars, sodium chloride, or polyalcohols such as mannitol and sorbitol, in the composition. Prolonged absorption of the injectable compositions can be achieved by including in the composition an agent which delays absorption, for example, aluminum monostearate or gelatin. In one embodiment, the pharmaceutically acceptable carrier is DMSO, alone or in combination with other carriers.

The therapeutically effective amount or dose of a compound of the present invention depends on the age, sex and weight of the patient, the current medical condition of the patient and the severity of the cancer in the patient being treated. The skilled artisan is able to determine appropriate doses depending on these and other factors.

The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

Doses of the compound of the invention for administration may be in the range of from about 1 μg to about 10,000 mg, from about 20 μg to about 9,500 mg, from about 40 μg to about 9,000 mg, from about 75 μg to about 8,500 mg, from about 150 μg to about 7,500 mg, from about 200 μg to about 7,000 mg, from about 3050 μg to about 6,000 mg, from about 500 μg to about 5,000 mg, from about 750 μg to about 4,000 mg, from about 1 mg to about 3,000 mg, from about 10 mg to about 2,500 mg, from about 20 mg to about 2,000 mg, from about 25 mg to about 1,500 mg, from about 30 mg to about 1,000 mg, from about 40 mg to about 900 mg, from about 50 mg to about 800 mg, from about 60 mg to about 750 mg, from about 70 mg to about 600 mg, from about 80 mg to about 500 mg, and any and all whole or partial increments therebetween.

In some embodiments, the dose of a compound of the invention is from about 1 mg to about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, the dosage of a second compound as described elsewhere herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

In one embodiment, the compositions of the invention are administered to the patient from about one to about five times per day or more. In various embodiments, the compositions of the invention are administered to the patient, 1-7 times per day, 1-7 times every two days, 1-7 times every 3 days, 1-7 times every week, 1-7 times every two weeks, and 1-7 times per month. It is readily apparent to one skilled in the art that the frequency of administration of the various combination compositions of the invention will vary from individual to individual depending on many factors including, but not limited to, age, the disease or disorder to be treated, the severity of the disease or disorder to be treated, gender, overall health, and other factors. Thus, the invention should not be construed to be limited to any particular dosing regime and the precise dosage and composition to be administered to any patient is determined by the medical professional taking all other factors about the patient into account.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, may be reduced to a level at which the improved disease is retained. In some embodiments, a patient may require intermittent treatment on a long-term basis, or upon any recurrence of the disease or disorder.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the present invention is directed to a packaged pharmaceutical composition comprising a container holding a therapeutically effective amount of a compound of the invention, alone or in combination with a second pharmaceutical agent; and instructions for using the compound to treat or prevent cancer in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, suitable forms include tablets, dragees, liquids, drops, suppositories, or capsules, caplets and gelcaps. The compositions formulated for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation involves the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of G-protein receptor-related diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release refers to a drug formulation that provides for gradual release of a drug over an extended period of time, and that may, although not necessarily, result in substantially constant blood levels of a drug over an extended time period. The period of time may be as long as a day, a week, or a month or more and should be a release which is longer that the same amount of agent administered in bolus form. The term delayed release is used herein in its conventional sense to refer to a drug formulation that provides for an initial release of the drug after some delay following drug administration and that mat, although not necessarily, includes a delay of from about 10 minutes up to about 12 hours.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term pulsatile release refers to a drug formulation that provides release of the drug in such a way as to produce pulsed plasma profiles of the drug after drug administration.

The term immediate release refers to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Therapeutic Utility

The compounds of the invention, subgroups and examples thereof, are inhibitors of Polq polymerase activity, and which may be useful in preventing or treating disease states or conditions described herein. In addition the compounds of the invention, and subgroups thereof, will be useful in preventing or treating diseases or condition mediated by Polq.

References to the preventing or prophylaxis or treatment of a disease state or condition such as cancer include within their scope alleviating or reducing the incidence of cancer. Thus, for example, it is envisaged that the compounds of the invention will be useful in alleviating or reducing the incidence of cancer.

In one embodiment, it is expected that compounds of Formula I (herein referred to as Polq inhibitors (Polqi)) will show preferential killing of BRCA deficient cells, or another words cancer cells that are defective in homology-directed repair (HDR). This is due to the fact that BRCA deficient ovarian cancer cells were shown to be dependent on Polq for their survival in the presence of genotoxic agents. This synthetic lethal relationship between Polq and HDR was further demonstrated in mouse models. Most importantly, the DNA synthesis activity of Polq was shown to promote the survival of BRCA deficient cells which strongly suggests that pharmacological inhibition of the polymerase domain by compounds presented herein will selectively kill BRCA deficient cancer cells which include but are not limited to cancers originating in the prostate, breast, ovary, pancreas.

Certain hematological cancers, including but not limited to acute myeloid leukemia (AML), have been shown to possess defects in HDR as a result of the effects of particular genetic mutations (i.e. BCR-ABL) or due to certain treatment regiments.

In one embodiment, Polqi presented herein may also be particularly effective in AML or other hematological cancers.

Several factors important for HDR may be defective and/or downregulated in cancer cells, including but not limited to Mre11, Rad50, Nbs1, CtIP, Exo1, PALB2, BARD1, RAD51B, RAD51C, RAD51D, XRCC2 and XRCC3. Therefore, in one embodiment, cancer cells defective or downregulated in one or more of these HDR factors will be susceptible to the Polq inhibitors described herein.

In one embodiment, said HDR genes are selected from any of: ATM, ATR, BRCA1, BRCA2, BARD1, RAD51C, RAD50, CHEK1, CHEK2, FANCA, FANCB, FANCC, FANCD2, FANCE, FANCF, FANCG, FANCI, FANCL, FANCM, PALB2 (FANCN), FANCP (BTBD12), ERCC4 (FANCQ), PTEN, CDK12, MRE1 1, NBS1, NBN, CLASPIN, BLM, WRN, SMARCA2, SMARCA4, LIG1, RPA1, RPA2, BRIP1 and PTEN.

Additionally, Polq inactivation in combination with DDR factors RAD54 or FANCJ also results in synthetic lethality. Thus, in one embodiment it is expected that Polqi described herein will preferentially kill cancer cells with defects or downregulation of RAD54 and/or FANCJ.

Furthermore, recent studies demonstrate that inactivation of Polq in combination with inhibition of the DDR factor ATR also results in a significant reduction in cell proliferation. Semi-synthetic lethality between Polq and ATM was also identified in earlier studies. Thus, in one embodiment it is expected that Polqi described herein will show preferential killing of cancer cells defective in or downregulated in ATR or ATM DDR factors. In one embodiment, it is also expected that Polqi described herein will exhibit synergistic or additive anti-proliferation effects when combined with ATR inhibitors or ATM inhibitors. In another embodiment, it is also expected that Polqi described herein will show effective killing of cancer cells exhibiting replicative stress, especially when combined with other anti-cancer agents that exacerbate replicative stress, including but not limited to gemcitabine, ATR inhibitors, cytarabine, topoisomerase inhibitors (i.e. etoposide), cisplatin, etc.

Polq was also shown to confer resistance to ionizing radiation (IR), bleomycin, cisplatin, mitomycin C, and topoisomerase inhibitors (etoposide, camptothecin). Therefore, in another embodiment it is expected that Polqi described herein will promote cancer cell sensitivity to a variety of anti-cancer agents including but not limited to IR, bleomycin, cisplatin, mitomycin C and topoisomerase inhibitors.

PARP inhibitors (PARPi) and Polq inactivation both reduce cancer cell resistance to IR. Thus, in another embodiment it is expected that combining Polqi described herein with PARPi will sensitize cancer cells to IR and overcome cancer cell resistance to IR. Furthermore, it has been shown that suppression of Polq combined with a DNApk inhibitor causes cancer cell sensitivity to IR. Thus, in another embodiment it is expected that Polqi described here in will show synergistic anti-proliferation effects when combined with DNApk inhibitors and IR or other anti-cancer agents that cause DNA double-strand breaks.

It has also been shown that suppression of Polq expression confers cellular sensitivity to PARP inhibition in HDR defective cancer cells. Therefore, in one embodiment it is expected that Polqi described herein will act synergistically with PARP inhibitors (PARPi), especially in HDR defective cells. In one embodiment, it is expected that Polqi combined with PARPi including but not limited to Lynparza (olaparib), talazoparib, niraparib, and rucaparib will potentiate the effects of PARPi in solid tumors and hematological malignancies. In one embodiment, is expected that Polqi described herein when combined with PARPi will suppress cancer cell resistance to PARPi.

In one embodiment, Polqi described herein are expected to induce synthetic lethality in cancer cells with defects in or suppression of the expression of non-homologous end-joining NHEJ factors such as LIG4 or KU70/80. In one embodiment, said non-homologous end-joining genes are selected from any one or more of: LIG4, NHEJ1, POLL, POLM, PRKDC, XRCC4, XRCC5, XRCC6, and DCLRE1 C. According to a further aspect of the invention there is a provided a compound of formula (I) as defined herein for use in the treatment of tumours which have elevated ligase Ilia levels, reduced ligase IV levels and increased dependence upon MMEJ (altEJ) DSB repair.

Suppression of Polq expression enhances and reduces the off-target effects of genome engineering by CRISPR-Cas9 type RNA-guided endonucleases, described in WO 2017/062754. Thus, in one embodiment it is expected that Polqi described herein will benefit CRISPR-Cas9 based genome engineering by reducing off-target effects and thus increase the fidelity and safety of CRISPR-Cas9 type RNA-guided genome engineering for therapeutics and basic research applications. In one embodiment, it is expected that combining Polqi described herein with DNApk inhibitors will have an even greater effect on increasing the fidelity and safety of CRISPR-Cas9 type RNA-guided genome engineering for therapeutic and basic research applications.

### EXPERIMENTAL EXAMPLES

Those skilled in the art recognize, or are able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Example 1

### 2-(3-(2-morpholinoethyl)-2-oxoimidazolidin-1-yl)-4,6-bis(trifluoromethyl)phenyl (4-fluorophenyl)(methyl)carbamate

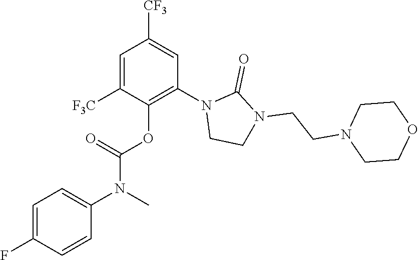 [SMILES: CN(C(=O)Oc1c(N2CCN(CCN3CCOCC3)C2=O)cc(C(F)(F)F)cc1C(F)(F)F)c1ccc(F)cc1]

### Step 1: Synthesis of (4-Fluorophenyl)(methyl)carbamic chloride

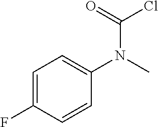

To a solution of 4-fluoro-N-methylaniline (0.58 g, 4.66 mmol) and pyridine (0.73 g, 9.34 mmol) in 12 mL of dichloromethane at 0 °C, triphosgene (0.69 g, 2.33 mmol) dissolved in 6 mL dichloromethane was added dropwise under inert atmosphere. The reaction was stirred at ambient temperature (room temperature) for 2 h. The reaction mixture was diluted with 20 mL dichloromethane and extracted with 20 mL 1N HCl. The organic layer was separated, dried over with anhydrous sodium sulfate, filtered and concentrated to a solid under reduced pressure. ¹H NMR (400 MHz, CDCl₃) δ 7.15 (m, 2H), 7.04 (m, 2H), 3.3 (s, 3H); MS(ESI): m/z 188.0 [(M+H)⁺].

### Step 2: Synthesis of 2-iodo-4,6-bis(trifluoromethyl)phenol

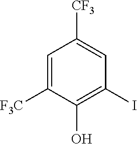 [SMILES: Oc1c(I)cc(C(F)(F)F)cc1C(F)(F)F]

A solution of 2,4-bis(trifluoromethyl)phenol (1.00 g, 4.35 mmol) in THF:H₂O (3:1, 24 mL) was cooled to 0 °C in an ice bath under nitrogen. Iodine (1.18 g, 4.64 mmol) and Na₂CO₃ (491 mg, 4.64 mmol) were added sequentially. The ice bath was removed and the reaction was allowed to warm to RT with stirring overnight. The reaction solution was cooled to 0 °C in an ice bath, quenched with saturated aqueous sodium metabisufite and stirred at 0 °C until all of the solution turned yellow in color. This mixture was extracted with EtOAc (3×). The combined organic extracts were washed with water and brine, dried over anhydrous sodium sulfate and concentrated. The crude product was purified by column chromatography on silica gel using a gradient solvent system of 0 to 10% of ethyl acetate in hexanes to afford the titled compound as colorless crystalline solid (711 mg, 46%). ¹H NMR (400 MHz, CDCl₃) δ 8.10 (s, 1H), 7.82 (s, 1H), 6.15 (s, 1H).

### Step 3: Synthesis of 2-iodo-4,6-bis(trifluoromethyl)phenyl (4-fluorophenyl)(methyl)carbamate

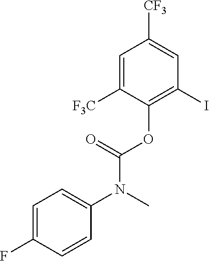 [SMILES: CN(C(=O)Oc1c(I)cc(C(F)(F)F)cc1C(F)(F)F)c1ccc(F)cc1]

2-iodo-4,6-bis(trifluoromethyl)phenol (570 mg, 1.60 mmol) and (4-fluorophenyl)(methyl)carbamic chloride (751 mg, 4.00 mmol) were dissolved into anhydrous pyridine (10 mL). This solution was stirred at 90 °C for 4 hours. The reaction was cooled to RT and concentrated down. The residual solid was partitioned between EtOAc and 1N aqueous HCl. The aqueous phase was separated and extracted with EtOAc twice. The combined organic extracts were washed with brine, dried over anhydrous sodium sulfate and concentrated. The crude product was purified by column chromatography on silica gel using a gradient solvent system of 0 to 20% of ethyl acetate in hexanes to afford the titled compound as a yellow oil (683 mg, 84%). ¹H NMR (400 MHz, CDCl₃) δ 8.21-8.27 (1H), 7.84-7.92 (1H), 7.39 (m, 2H), 7.12 (m, 2H), 3.38-3.56 (3H); ESIMS: m/z 508.0 [(M+H)⁺]

### Step 4: Synthesis of 1-(2-morpholinoethyl)imidazolidin-2-one

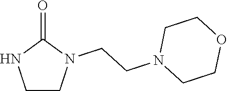 [SMILES: O=C1NCCN1CCN1CCOCC1]

A solution of 1-(2-chloroethyl)-2-imidazolidinone (0.673 mmol, 100 mg) in morpholine (1.0 mL) was microwaved at 120 °C for one hour. The reaction was concentrated down and the residual solid was partially dissolved into dichloromethane. Basic ion exchange resin was added and this mixture was stirred at room temperature for one hour. The resin was filtered off and the filtrate was concentrated to afford the titled compound as a yellow solid (101 mg, 75%). ¹H NMR (400 MHz, CDCl₃) δ 4.34 (s, 1H), 3.73 (s, 4H), 3.53 (m, 2H), 3.42 (m, 2H), 3.36 (m, 2H), 2.54 (s, 6H); ESIMS: m/z 421.3 [(2M+Na)⁺].

### Step 5: Synthesis of 2-(3-(2-morpholinoethyl)-2-oxoimidazolidin-1-yl)-4,6-bis(trifluoromethyl)phenyl (4-fluorophenyl)(methyl)carbamate

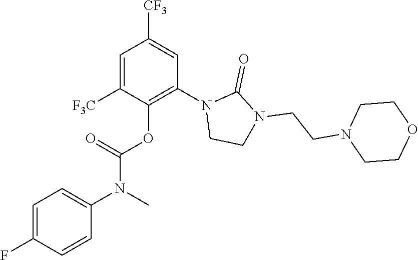 [SMILES: CN(C(=O)Oc1c(N2CCN(CCN3CCOCC3)C2=O)cc(C(F)(F)F)cc1C(F)(F)F)c1ccc(F)cc1]

2-iodo-4,6-bis(trifluoromethyl)phenyl (4-fluorophenyl)(methyl)carbamate (0.1972 mmol, 100 mg), 1-(2-morpholinoethyl)imidazolidin-2-one (0.3539 mmol, 70.5 mg), copper (I) iodide (0.0961 mmol, 18.3 mg), cesium fluoride (0.3995 mmol, 60.7 mg), N,N′-dimethylethylenediamine (0.1972 mmol, 21.2 uL) and anhydrous powered potassium carbonate (0.3641 mmol, 50.3 mg) were added to degassed anhydrous 1,4-dioxane (3.0 mL) under nitrogen. The resulting suspension was stirred at 90 °C overnight. The reaction was cooled to room temperature, diluted with methanol and filtered. The clear filtrate was concentrated down to yield a semi-solid. This was suspended in dichloromethane, filtered through plug of celite and concentrated down. The crude product was purified by column chromatography on silica gel using a gradient solvent system of 0 to 10% of methanol in ethyl acetate to afford the titled compound as a yellow oil (13.9 mg, 12%). ¹H NMR (400 MHz, CDCl₃) δ 7.66-8.02 (2H), 7.32 (m, 2H), 7.09 (m, 2H), 3.31-4.04 (15H), 2.54 (m, 4H); ESIMS: m/z 579.2 [(M+H)⁺].

### Example 2

### 2-(3-(2-(4-acetylpiperazin-1-yl)ethyl)-2-oxoimidazolidin-1-yl)-4,6-bis(trifluoromethyl)phenyl (4-fluorophenyl)methyl)carbamate

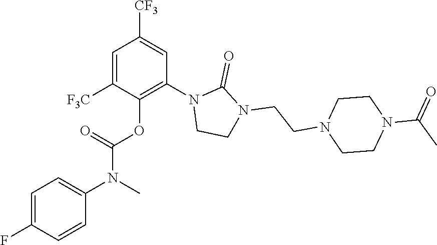 [SMILES: CC(=O)N1CCN(CCN2CCN(c3cc(C(F)(F)F)cc(C(F)(F)F)c3OC(=O)N(C)c3ccc(F)cc3)C2=O)CC1]

### Step 1: Synthesis of 1-(2-(4-acetylpiperazin-1-yl)ethyl)imidazolidin-2-one

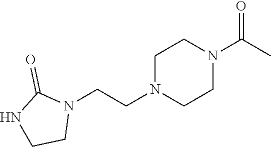 [SMILES: CC(=O)N1CCN(CCN2CCNC2=O)CC1]

A suspension of 1-(2-chloroethyl)-2-imidazolidinone (3.365 mmol, 500 mg), 1-acetylpiperazine (4.038 mmol, 518 mg) and powdered anhydrous potassium carbonate (4.038 mmol, 558 mg) in anhydrous dimethylacetamide (5 mL) was microwaved at 120 °C for one hour. The solids were filtered off and washed with DMA. The clear filtrate was concentrated down. The crude product was purified by column chromatography on silica gel using a gradient solvent system of 0 to 100% of solvent B (10% of [2N NH₃ in MeOH) in DCM) in solvent A (DCM) to afford the titled compound as a pale orange crystalline solid (620 mg, 77%). ¹H NMR (400 MHz, DMSO-d₆) δ 6.24 (s, 1H), 3.35 (m, 5H), 3.19 (m, 5H), 2.36 (m, 6H), 1.97 (m, 3H); ESIMS: m/z 241.2 [(M+H)⁺].

### Step 2: Synthesis of 2-(3-(2-(4-acetylpiperazin-1-yl)ethyl)-2-oxoimidazolidin-1-yl)-4,6-bis(trifluoromethyl)phenyl (4-fluorophenyl)(methyl)carbamate

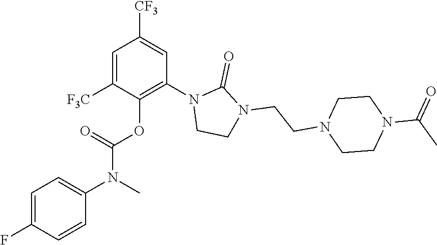 [SMILES: CC(=O)N1CCN(CCN2CCN(c3cc(C(F)(F)F)cc(C(F)(F)F)c3OC(=O)N(C)c3ccc(F)cc3)C2=O)CC1]

2-iodo-4,6-bis(trifluoromethyl)phenyl (4-fluorophenyl)(methyl)carbamate (0.3944 mmol, 200 mg), 1-(2-(4-acetylpiperazin-1-yl)ethyl)imidazolidin-2-one (0.7078 mmol, 170 mg), copper (I) iodide (0.1922 mmol, 37 mg), cesium fluoride (0.7990 mmol, 121 mg), N,N′-dimethylethylenediamine (0.3944 mmol, 43 uL) and anhydrous powered potassium carbonate (0.7282 mmol, 101 mg) were added to degassed anhydrous 1,4-dioxane (4.0 mL) under nitrogen. The resulting suspension was stirred at 90 °C overnight. The reaction was cooled to room temperature, diluted with methanol and filtered. The clear filtrate was concentrated down to yield a semi-solid. This was suspended in dichloromethane, filtered through plug of celite and concentrated down. The crude product was purified by column chromatography on silica gel using a gradient solvent system of 0 to 100% of (10% MeOH in EtOAc) in hexanes to elute the byproducts. The solvent system was then changed to (10% of [2N NH₃ in MeOH] in DCM) to elute and afford the titled compound as a pale yellow glassy solid (25.6 mg, 10%).

¹H NMR (400 MHz, CDCl₃) δ 7.66-8.04 (2H), 7.32 (m, 2H), 7.09 (m, 2H), 3.29-4.05 (14H), 2.32-2.84 (5H), 2.08 (s, 3H); ESIMS: m/z 620.2 [(M+H)⁺].

### Example 3

### 2-(3-(2-(4-cyanopiperidin-1-yl)ethyl)-2-oxoimidazolidin-1-yl)-4,6-bis(trifluoromethyl)phenyl (4-fluorophenyl)(methyl)carbamate

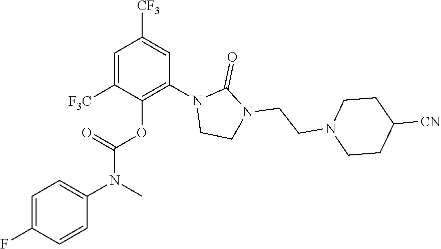 [SMILES: CN(C(=O)Oc1c(N2CCN(CCN3CCC(C#N)CC3)C2=O)cc(C(F)(F)F)cc1C(F)(F)F)c1ccc(F)cc1]

### Step 1: Synthesis of 1-(2-(2-oxoimidazolidin-1-yl)ethyl)piperidine-4-carbonitrile

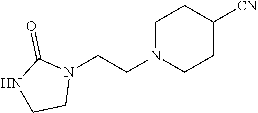 [SMILES: N#CC1CCN(CCN2CCNC2=O)CC1]

A suspension of 1-(2-chloroethyl)-2-imidazolidinone (3.365 mmol, 500 mg), piperidine-4-carbonitrile (4.038 mmol, 454 uL) and powdered anhydrous potassium carbonate (4.038 mmol, 558 mg) in anhydrous dimethylacetamide (5 mL) was microwaved at 120 °C for one hour. The solids were filtered off and washed with DMA. The clear filtrate was concentrated down. The crude product was purified by column chromatography on silica gel using a gradient solvent system of 0 to 10% of MeOH in DCM to afford the titled compound as an off-white solid (265 mg, 35%).

¹H NMR (400 MHz, DMSO-d₆) δ 6.22 (s, 1H), 3.34 (m, 2H), 3.19 (t, J=8.12 Hz, 2H), 3.12 (t, J=6.68 Hz, 2H), 2.84 (m, 1H), 2.55 (m, 2H), 2.36 (t, J=6.68 Hz, 2H), 2.27 (m, 2H), 1.82 (m, 2H), 1.66 (m, 2H); ESIMS: m/z 223.1 [(M+H)⁺].

### Step 2: Synthesis of 2-(3-(2-(4-cyanopiperidin-1-yl)ethyl)-2-oxoimidazolidin-1-yl)-4,6-bis(trifluoromethyl)phenyl (4-fluorophenyl)(methyl)carbamate

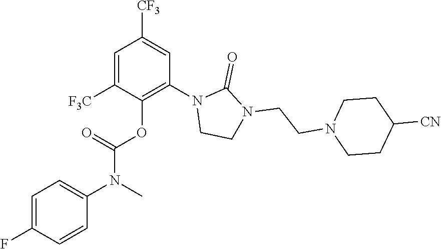 [SMILES: CN(C(=O)Oc1c(N2CCN(CCN3CCC(C#N)CC3)C2=O)cc(C(F)(F)F)cc1C(F)(F)F)c1ccc(F)cc1]

2-iodo-4,6-bis(trifluoromethyl)phenyl (4-fluorophenyl)(methyl)carbamate (0.3944 mmol, 200 mg), 1-(2-(2-oxoimidazolidin-1-yl)ethyl)piperidine-4-carbonitrile (0.7078 mmol, 157 mg), copper (I) iodide (0.192 mmol, 37 mg), cesium fluoride (0.7990 mmol, 121 mg), N,N′-dimethylethylenediamine (0.3944 mmol, 43 uL) and anhydrous powered potassium carbonate (0.7282 mmol, 101 mg) were added to degassed anhydrous 1,4-dioxane (4.0 mL) under nitrogen. The resulting suspension was stirred at 90 °C overnight. The reaction was cooled to room temperature, diluted with methanol and filtered. The clear filtrate was concentrated down to yield a semi-solid. This was suspended in dichloromethane, filtered through plug of celite and concentrated down. The crude product was purified by column chromatography on silica gel using a gradient solvent system of 0 to 100% of (10% MeOH in EtOAc) in hexanes to afford the titled compound as a colorless oil (18.6 mg, 8%). ¹H NMR (400 MHz, CDCl₃) δ 7.66-8.04 (2H), 7.31 (m, 2H), 7.09 (m, 2H), 3.30-3.99 (10H), 2.25-2.96 (7H), 1.91 (m, 3H); ESIMS: m/z 602.2 [(M+H)⁺].

### Example 4

### 2,4-bis(trifluoromethyl)-6-(2-oxoimidazolidin-1-yl)phenyl (4-fluorophenyl)(methyl)carbamate

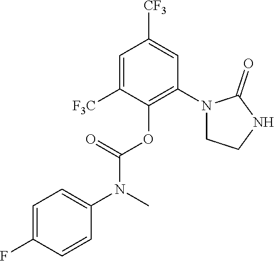 [SMILES: CN(C(=O)Oc1c(N2CCNC2=O)cc(C(F)(F)F)cc1C(F)(F)F)c1ccc(F)cc1]

2-iodo-4,6-bis(trifluoromethyl)phenyl (4-fluorophenyl)(methyl)carbamate (0.39 mmol, 200 mg), 2-imidazolidone (0.7 mmol, 64 mg), copper (I) iodide (0.19 mmol, 37 mg), cesium fluoride (0.79 mmol, 120 mg), N,N′-dimethylethylenediamine (0.39 mmol, 43 uL) and anhydrous powered potassium carbonate (0.72 mmol, 100 mg) were added to degassed anhydrous 1,4-dioxane (16.0 mL) under nitrogen. The resulting suspension was stirred at 90 °C overnight. The reaction was cooled to room temperature and concentrated down to yield a semi-solid. The crude product was purified by column chromatography on silica gel using a gradient solvent system of 0 to 3% of methanol in dichloromethane to afford the titled compound as a off white solid. Repurified on 20×20 cm silica gel 60 GF 254, 1 mm PLC glass plate, 2% methanol in dichloromethane as eluent to afford 28 mg of white solid. (28 mg, 15%).

¹H NMR (500 MHz) δ 8.39 (m, 2H), 7.75 (m, 2H), 7.54 (m, 2H), 4.52 (m, 2H), 4.32 (m, 2H), 3.91 (m, 3H); ESIMS: m/z 465.05 [(M+H)⁺].

### Example 5

### 2,4-bis(trifluoromethyl)-6-(3-methyl-2-oxoimidazolidin-1-yl)phenyl (4-fluorophenyl)(methyl)carbamate

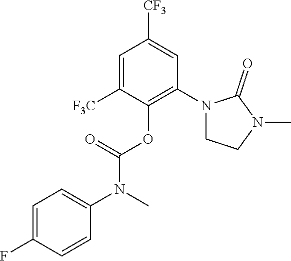 [SMILES: CN1CCN(c2cc(C(F)(F)F)cc(C(F)(F)F)c2OC(=O)N(C)c2ccc(F)cc2)C1=O]

2-iodo-4,6-bis(trifluoromethyl)phenyl (4-fluorophenyl)(methyl)carbamate (0.1972 mmol, 100 mg), 1-Methyl-2-imidazolidinone (0.39 mmol, 39 mg), copper (I) iodide (0.0961 mmol, 18.3 mg), cesium fluoride (0.3995 mmol, 60.7 mg), N,N′-dimethylethylenediamine (0.1972 mmol, 21.2 uL) and anhydrous powered potassium carbonate (0.3641 mmol, 50.3 mg) were added to degassed anhydrous 1,4-dioxane (5.0 mL) under nitrogen. The resulting suspension was stirred at 90 °C overnight. The reaction was cooled to room temperature, filtered and the filtrate was concentrated down to yield a semi-solid. The crude product was purified by column chromatography on silica gel using a gradient solvent system of 0 to 100% of ethyl acetate in hexanes to afford the titled compound as a yellow oil (2 mg, 2%).

¹H NMR (400 MHz, CDCl₃) δ 7.86-7.7 (m, 2H), 7.32 (m, 2H), 7.09 (m, 2H), 3.70 (m, 2H), 3.51 (m, 2H), 3.45 (d, J=44 Hz, 3H), 2.92 (s, 3H); ESIMS: m/z 480.14 [(M+H)⁺].

### Example 6

### 2,4-bis(trifluoromethyl)-6-(3-(2-hydroxyethyl)-2-oxoimidazolidin-1-yl)phenyl (4-fluorophenyl)(methyl)carbamate

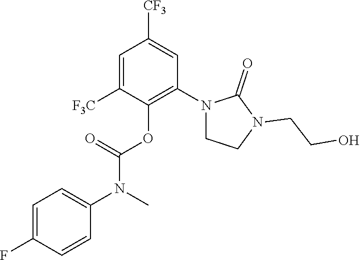 [SMILES: CN(C(=O)Oc1c(N2CCN(CCO)C2=O)cc(C(F)(F)F)cc1C(F)(F)F)c1ccc(F)cc1]

2-iodo-4,6-bis(trifluoromethyl)phenyl (4-fluorophenyl)(methyl)carbamate (0.1972 mmol, 100 mg), 1-(2-Hydroxyethyl-2-imidazolidinone (0.39 mmol, 51 mg), copper (I) iodide (0.0961 mmol, 18.3 mg), cesium fluoride (0.3995 mmol, 60.7 mg), N,N′-dimethylethylenediamine (0.1972 mmol, 21.2 uL) and anhydrous powered potassium carbonate (0.3641 mmol, 50.3 mg) were added to degassed anhydrous 1,4-dioxane (5.0 mL) under nitrogen. The resulting suspension was stirred at 90 °C overnight. The reaction was cooled to room temperature, filtered and the filtrate was concentrated down to yield a semi-solid. The crude product was purified by column chromatography on silica gel using a gradient solvent system of 0 to 100% of ethyl acetate in hexanes to afford the titled compound as a yellow oil (11 mg, 11%).

¹H NMR (400 MHz, CDCl₃) δ 7.81 (m, 2H), 7.32 (m, 2H), 7.09 (m, 2H), 3.85 (s, 3H), 3.70 (s, 1H), 3.61 (m, 2H), 3.47 (m, 4H), 3.36 (m, 2H); ESIMS: m/z 510.48 [(M+H)⁻].

### Example 7

### 2,4-bis(trifluoromethyl)-6-(2-oxoimidazolidin-1-yl)phenyl (3-chloro-2,4-difluorophenyl)(methyl)carbamate

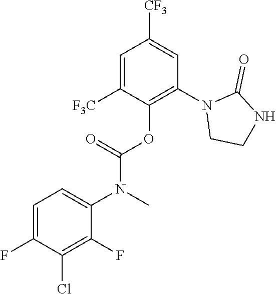 [SMILES: CN(C(=O)Oc1c(N2CCNC2=O)cc(C(F)(F)F)cc1C(F)(F)F)c1ccc(F)c(Cl)c1F]

### Step 1: Synthesis of (3-chloro-2,4-difluorophenyl)(methyl)carbamic chloride

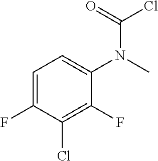

To a solution of 3-chloro-2,4-fluoro-N-methylaniline (0.827 g, 4.66 mmol) and pyridine (0.73 g, 9.34 mmol) in 12 mL of dichloromethane at 0 °C, triphosgene (0.69 g, 2.33 mmol) dissolved in 6 mL dichloromethane was added dropwise under inert atmosphere. The reaction was stirred at ambient temperature (room temperature) for 2 h. The reaction mixture was diluted with 20 mL dichloromethane and extracted with 20 mL 1N HCl. The organic layer was separated, dried over with anhydrous sodium sulfate, filtered and concentrated to a solid under reduced pressure.

ESIMS: m/z 240.09 [(M+H)⁺].

### Step 2: Synthesis of 2,4-bis(trifluoromethyl)-6-iodophenyl (2-chloro-3,4-difluorophenyl)(methyl)carbamate

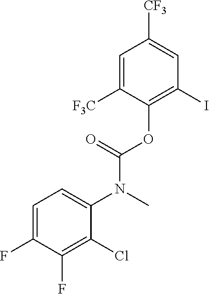 [SMILES: CN(C(=O)Oc1c(I)cc(C(F)(F)F)cc1C(F)(F)F)c1ccc(F)c(F)c1Cl]

2-iodo-4,6-bis(trifluoromethyl)phenol (570 mg, 1.60 mmol) and (3-chloro-2,4-difluorophenyl)(methyl)carbamic chloride (300 mg, 0.84 mmol) were dissolved into anhydrous pyridine (5 mL). This solution was stirred at 90 °C for 4 hours. The reaction was cooled to RT and concentrated down. The residual solid was partitioned between EtOAc and 1N aqueous HCl. The aqueous phase was separated and extracted with EtOAc twice. The combined organic extracts were washed with brine, dried over anhydrous sodium sulfate and concentrated. The crude product was purified by column chromatography on silica gel using a gradient solvent system of 0 to 20% of ethyl acetate in hexanes to afford the titled compound as a yellow oil (227 mg, 48%) ESMS: m/z 560.21 [(M+H)⁺].

### Step 3: Synthesis of 2,4-bis(trifluoromethyl)-6-(2-oxoimidazolidin-1-yl)phenyl (3-chloro-2,4-difluorophenyl)(methyl)carbamate

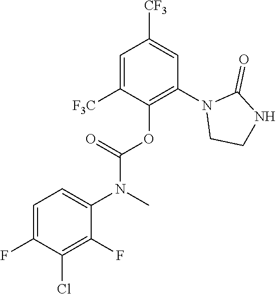 [SMILES: CN(C(=O)Oc1c(N2CCNC2=O)cc(C(F)(F)F)cc1C(F)(F)F)c1ccc(F)c(Cl)c1F]

2,4-bis(trifluoromethyl)-6-iodophenyl (2-chloro-3,4-difluorophenyl)(methyl)carbamate (0.2269 mmol, 126 mg), 2-imidazolidone (0.4548 mmol, 39 mg), copper (I) iodide (0.11135 mmol, 22 mg), cesium fluoride (0.4538 mmol, 69 mg), N,N′-dimethylethylenediamine (0.2269 mmol, 25 uL) and anhydrous powered potassium carbonate (0.4538 mmol, 63 mg) were added to degassed anhydrous 1,4-dioxane (5.0 mL) under nitrogen. The resulting suspension was stirred at 90 °C overnight. The reaction was cooled to room temperature, filtered and the filtrate was concentrated down to yield a semi-solid. The crude product was purified by column chromatography on silica gel using a gradient solvent system of 0 to 100% of ethyl acetate in hexanes to afford the titled compound as a yellow oil (6 mg, 5%). ¹H NMR (400 MHz, CDCl₃) S 7.83 (m, 2H), 7.19 (m, 2H), 3.88 (m, 2H), 3.64 (t, J=12 Hz, 2H), 3.4 (d, J=56 Hz, 3H), 3.21 (s, 1H); ESIMS: m/z 518.3 [(M+H)⁺].

### Example 8

### 2,4-bis(trifluoromethyl)-6-(3-methyl-2-oxoimidazolidin-1-yl)phenyl (3-chloro-2,4-difluorophenyl)(methyl)carbamate

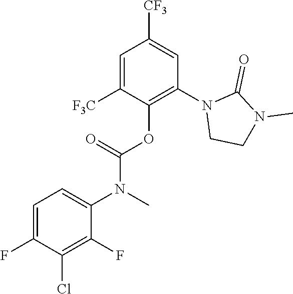 [SMILES: CN1CCN(c2cc(C(F)(F)F)cc(C(F)(F)F)c2OC(=O)N(C)c2ccc(F)c(Cl)c2F)C1=O]

2,4-bis(trifluoromethyl)-6-iodophenyl (5-chloro-2,4-difluorophenyl)(methyl)carbamate (0.276 mmol, 140 mg), 1-methyl-2-imidazolidione (0.552 mmol, 55 mg), copper (I) iodide (0.136 mmol, 26 mg), cesium fluoride (0.552 mmol, 84 mg), N,N′-dimethylethylenediamine (0.276 mmol, 30 μL) and anhydrous powered potassium carbonate (0.552 mmol, 76 mg) were added to degassed anhydrous 1,4-dioxane (5.0 mL) under nitrogen. The resulting suspension was stirred at 90 °C overnight. The reaction was cooled to room temperature, filtered and the filtrate was concentrated down to yield a semi-solid. The crude product was purified by column chromatography on silica gel using a gradient solvent system of 0 to 100% of ethyl acetate in hexanes to afford the titled compound as a yellow oil (7 mg, 5%).

¹H NMR (400 MHz, CDCl₃) δ 7.81 (m, 2H), 7.30 (m, 1H), 7.05 (m, 1H), 3.72 (m, 2H), 3.51 (m, 2H), 3.38 (d, J=52 Hz, 3H), 2.93 (s, 3H); ESIMS: m/z 532.3 [(M+H)⁺].

### Example 9

### 2,4-bis(trifluoromethyl)-6-(2-oxoimidazolidin-1-yl)phenyl (5-chloro-2,4-difluorophenyl)(methyl)carbamate

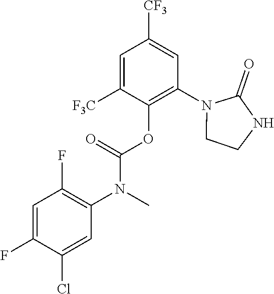 [SMILES: CN(C(=O)Oc1c(N2CCNC2=O)cc(C(F)(F)F)cc1C(F)(F)F)c1cc(Cl)c(F)cc1F]

### Step 1: Synthesis of (5-chloro-2,4-difluorophenyl)(methyl)carbamic chloride

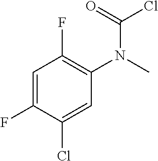

To a solution of 5-chloro-2,4-fluoro-N-methylaniline (0.827 g, 4.66 mmol) and pyridine (0.73 g, 9.34 mmol) in 12 mL of dichloromethane at 0 °C, triphosgene (0.69 g, 2.33 mmol) dissolved in 6 mL dichloromethane was added dropwise under inert atmosphere. The reaction was stirred at ambient temperature (room temperature) for 2 h. The reaction mixture was diluted with 20 mL dichloromethane and extracted with 20 mL 1N HCL. The organic layer was separated, dried over with anhydrous sodium sulfate, filtered and concentrated to a solid under reduced pressure.

ESIMS: m/z 225.42 [(M−35+22+H)⁺].

### Step 2: Synthesis of 2,4-bis(trifluoromethyl)-6-iodophenyl (5-chloro-2,4-difluorophenyl)(methyl)carbamate

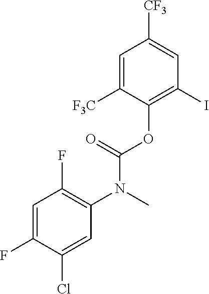 [SMILES: CN(C(=O)Oc1c(I)cc(C(F)(F)F)cc1C(F)(F)F)c1cc(Cl)c(F)cc1F]

Same experimental procedure as step 3 of example 1. ESIMS: m/z 559.91 [(M+H)⁺].

### Step 3: Synthesis of 2,4-bis(trifluoromethyl)-6-(2-oxoimidazolidin-1-yl)phenyl (5-chloro-2,4-difluorophenyl)(methyl)carbamate

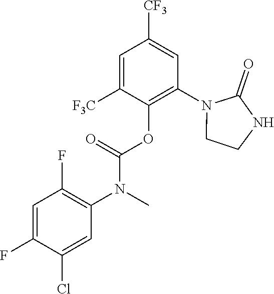 [SMILES: CN(C(=O)Oc1c(N2CCNC2=O)cc(C(F)(F)F)cc1C(F)(F)F)c1cc(Cl)c(F)cc1F]

2-iodo-4,6-bis(trifluoromethyl)phenyl (2,4-difluoro-5-chlorophenyl)(methyl)carbamate 0.276 mmol, 140 mg), 2-imidazolidone (0.552 mmol, 48 mg), copper (I) iodide (0.136 mmol, 26 mg), cesium fluoride (0.552 mmol, 84 mg), N,N′-dimethylethylenediamine (0.276 mmol, 30 uL) and anhydrous powered potassium carbonate (0.552 mmol, 76 mg) were added to degassed anhydrous 1,4-dioxane (5.0 mL) under nitrogen. The resulting suspension was stirred at 90 °C overnight. The reaction was cooled to room temperature, filtered and the filtrate was concentrated down to yield a semi-solid. The crude product was purified by column chromatography on silica gel using a gradient solvent system of 0 to 100% of ethyl acetate in hexanes to afford the titled compound as a yellow oil (4 mg, 3%).

(¹H NMR (400 MHz, CDCl₃) δ7.82 (m, 2H), 7.50 (m, 1H), 7.05 (m, 1H), 3.86 (m, 2H), 3.63 (t, J=8 Hz, 2H), 3.39 (d, J=56 Hz, 3H); ESIMS: m/z 518.32 [(M+H)⁺].

### Example 10

### 2,4-bis(trifluoromethyl)-6-(2-oxopyrrolidin-1-yl)phenyl (4-fluorophenyl)(methyl)carbamate

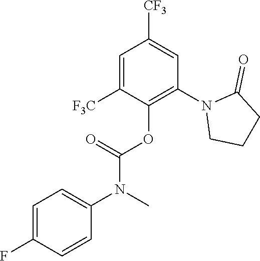 [SMILES: CN(C(=O)Oc1c(N2CCCC2=O)cc(C(F)(F)F)cc1C(F)(F)F)c1ccc(F)cc1]

2-bromo-4,6-bis(trifluoromethyl)phenyl (4-fluorophenyl)(methyl)carbamate (0.152 mmol, 70 mg), 2-Pyrrolidone (0.304 mmol, 23 uL), copper (I) iodide (0.076 mmol, 14 mg), N,N′-dimethylethylenediamine (0.152 mmol, 18 uL) and anhydrous powered potassium carbonate (0.465 mmol, 63 mg) were added to degassed anhydrous 1,4-dioxane (10 mL) under nitrogen. The resulting suspension was stirred at 90 °C overnight. The reaction was cooled to room temperature, diluted with methanol and filtered. The clear filtrate was concentrated down to yield a semi-solid. This was suspended in dichloromethane, filtered through plug of celite and concentrated down. The crude product was purified by column chromatography on silica gel using a gradient solvent system of 0 to 100% of ethyl acetate in hexanes to afford the titled compound as a yellow oil (5 mg, 7%). ¹H NMR (400 MHz, CDCl₃) δ 7.75 (m, 2H), 7.29 (m, 2H), 7.10 (m, 2H), 3.80 (m, 2H), 3.51 (m, 2H), 3.42 (d, J=56 Hz, 3H), 2.57 (s, 3 m, 2H) 2.22 (m, 2H); ESIMS: m/z 465.34 [(M+H)⁺].

### Example 11

### 2,4-bis(trifluoromethyl)-6-(pyrrolidin-1-yl)phenyl 4-fluorophenylmethylcarbamate

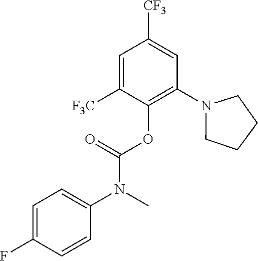 [SMILES: CN(C(=O)Oc1c(N2CCCC2)cc(C(F)(F)F)cc1C(F)(F)F)c1ccc(F)cc1]

### Step 1: Synthesis of 1-(2-(benzyloxy)-3,5-bis(trifluoromethyl)phenyl)pyrrolidine

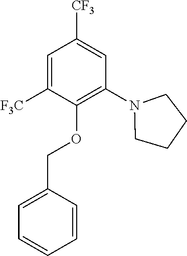 [SMILES: FC(F)(F)c1cc(N2CCCC2)c(OCc2ccccc2)c(C(F)(F)F)c1]

1-((2-bromo-4,6-bis(trifluoromethyl)phenoxy)methyl)benzene (0.25 g, 0.626 mmol), pyrrolidine (0.223 g, 3.13 mmol), [2,2′-Bis(diphenylphosphino)-1,1′-binaphthyl](0.058 g, 0.09 mmol), Palladium(0) bis(dibenzylideneacetone)(0.057 g, 0,062 mmol), Sodium tert-butoxide (0,073 g, 0.75 mmol) in 10 mL toluene were staired under inert atmosphere at 96 °C for 20 h. The reaction was cooled to room temperature and filtered through plug of celite and concentrated down. The crude product was purified by column chromatography on silica gel using a gradient solvent system of 0 to 20% of ethyl acetate in hexanes to afford 0.100 g the titled compound as a yellow oil. ESIMS: m/z 390.36 [(M+H)⁺].

### Step 2: Synthesis of 2,4-bis(trifluoromethyl)-6-(pyrrolidin-1-yl)phenol

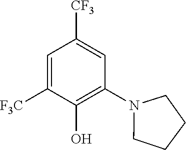 [SMILES: Oc1c(N2CCCC2)cc(C(F)(F)F)cc1C(F)(F)F]

1-(2-(benzyloxy)-3,5-bis(trifluoromethyl)phenyl)pyrrolidine (0.100 g, 0.25 mmol) in 10 mL methanol Pd/C (0.03 g, 0.28 mmol) was added under inert atmosphere. The reaction flask was evacuated of air and stirred under Hydrogen balloon for 20 h. The reaction was evacuated of hydrogen, purged with nitrogen gas, filtered and concentrated down to 0.068 g of title compound as an oil. ESIMS: m/z 301.39 [(M+H)⁺].

### Step 3: Synthesis of 2,4-bis(trifluoromethyl)-6-(pyrrolidin-1-yl)phenyl 4-fluorophenylmethylcarbamate

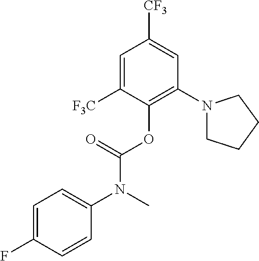 [SMILES: CN(C(=O)Oc1c(N2CCCC2)cc(C(F)(F)F)cc1C(F)(F)F)c1ccc(F)cc1]

2,4-bis(trifluoromethyl)-6-(pyrrolidin-1-yl)phenol (0.065 g, 0.21 mmol) in 10 mL acetonitrile, anhydrous potassium carbonate (0.06 g, 0.41 mmol) was added and stirred for 30 minutes. (4-fluorophenyl) (methyl)carbamic chloride (0.04 g, 0.21 mmol) was added to the reaction and stirred for 18 h. The reaction mixture was filtered, and the filtrate was concentrated under reduced pressure to give an oil. The crude product was purified by flash column chromatography on silica gel using 0-100% ethyl acetate in hexanes as eluent to afford 0.05 g as an off-white solid. ¹H NMR (400 MHz, CDCl₃) δ 7.32 (m, 2H), 7.10 (m, 4H), 7.3 (bs, 2H), 3.54 (m, 2H), 3.40 (m, 2H), 3.36 (s, 3H), 2.05 (m, 2H); MS(ESI): m/z 451.38

### Example 12

### (R)-2-(4-hydroxy-2-oxopyrrolidin-1-yl)-6-methyl-4-(trifluoromethyl)phenyl (4-fluorophenyl)(methyl)carbamate

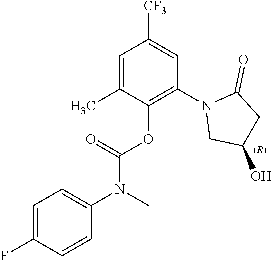

### Step 1: Synthesis of 4,4,5,5-tetramethyl-2-(2-methyl-4-(trifluoromethyl)phenyl)-1,3,2-dioxaborolane

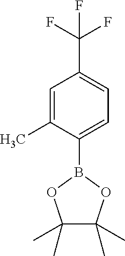

A solution of 1-bromo-2-methyl-4-(trifluoromethyl)benzene (1 g, 0.0041 mol) in 1,4 dioxane (20 mL) was purged with N2 in a sealed tube for 30 min and then added bispinacolato diboron (2.12 g, 0.00837 mol), potassium acetate (0.82 g, 0.00836 mol) and Pd(dppf)Cl₂·DCM (0.34 g, 0.00042 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (5×50 mL); washings collected was concentrated under reduced pressure to afford the yellow sticky solid (crude 3 g, Quantitative). The product was taken as such to next step without further purification.

### Step 2: Synthesis of 2-methyl-4-(trifluoromethyl)phenol

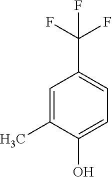

To a cold (0 °C) solution of 4,4,5,5-tetramethyl-2-(2-methyl-4-(trifluoromethyl)phenyl)-1,3,2-dioxaborolane (crude 6 g) in EtOH (50 mL), added hydrogen peroxide 30% aq. solution (4.1 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×30 mL). The organic layer collected was washed with water followed by brine, dried over anhydrous Na₂SO₄, filtered and concentrated under reduced pressure to afford the crude. The crude obtained was purified by column chromatography (100-200 silica gel, 10-15% EtOAc in Hexane as eluent) to afford the title compound as pale yellow liquid (0.7 g, 95% yield).

### Step 3: Synthesis of 2-iodo-6-methyl-4-(trifluoromethyl)phenol

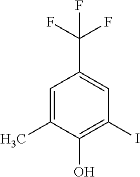

A solution of 2-methyl-4-(trifluoromethyl)phenol (0.7 g, 0.00397 mol) in THF:H₂O (3:1, 19 mL) was cooled 0 °C in an ice bath with stirring. After 15 minutes, was added 12 (1.1 g, 0.00433 mol) followed by Na₂CO₃ (0.46 g, 0.00433 mol) under inert atmosphere with stirring. Allowed the reaction mixture to stir at ambient temperature for 24 h. After completion of the reaction (TLC: R_{f} −0.35, 10% EtOAc in Hexane), the reaction mixture was cooled to 0 °C and then quenched with aq. sodium metabisulfite solution followed by extraction with EtOAc (2×50 mL). The organic layer collected was washed with water followed by brine, dried over anhydrous Na₂SO₄, filtered and concentrated under reduced pressure to afford the crude. The crude obtained was purified by column chromatography (100-200 silica gel, 0-5% EtOAc in Hexane as eluent) to afford the title compound as pale yellow liquid (1.0 g, 83% yield).

### Step 4: Synthesis of (4-fluorophenyl)(methyl)carbamic chloride

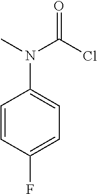

To a cold (0 °C) solution of triphosgene (0.57 g, 0.002 mol) in DCM (15 mL) added a solution of N-methyl-4-fluoro aniline (0.5 g, 0.0039 mol) and pyridine (0.61 g, 0.62 mL, 0.0078 mol) dropwise for over a period of 10 min. After that continued stirring at RT for 16 h. Progress of the reaction was monitored by TLC. After completion of the reaction, quenched the reaction mixture with 1M aq·HCl (15 mL) and then extracted with DCM (2×40 mL). The DCM layer separated was dried over anhydrous Na₂SO₄, filtered, and concentrated under reduced pressure to afford the title compound as green solid (0.7 g, 93%).

### Step 5: Synthesis of 2-iodo-6-methyl-4-(trifluoromethyl)phenyl (4-fluorophenyl)(methyl)carbamate

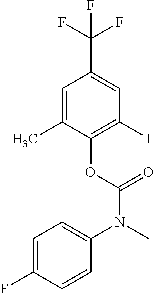 [SMILES: Cc1cc(C(F)(F)F)cc(I)c1OC(=O)N(C)c1ccc(F)cc1]

To a stirred solution of 2-iodo-6-methyl-4-(trifluoromethyl)phenol,5-difluorophenol (1 g, 0.0033 mol) in DMF (15 mL) at RT added K2CO₃ (0.91 g, 0.0066 mol) and continued stirring at RT for 30 minutes. To the above mixture added a solution of (4-fluorophenyl)methyl)carbamic chloride (0.62 g, 0.0033 mol) in DCM (15 mL) dropwise. The resultant reaction mixture was further stirred at RT for 7 h. After completion of the reaction was confirmed by TLC (R_{f} −0.25, 5% EtOAc in Hexane) quenched the reaction mixture with cold water (15 mL) and extracted with EtOAc (2×40 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off white solid (0.8 g, 53%).

### Step 6: Synthesis of (R)-2-(4-hydroxy-2-oxopyrrolidin-1-yl)-6-methyl-4-(trifluoromethyl)phenyl (4-fluorophenyl)(methyl)carbamate

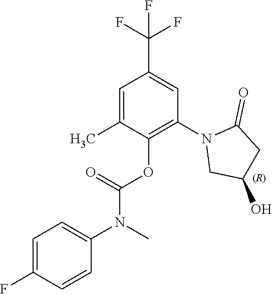

2-iodo-6-methyl-4-(trifluoromethyl)phenyl (4-fluorophenyl)(methyl)carbamate (0.3 g, 0.000662 mol), (R)-4-hydroxypyrrolidin-2-one (0.133 g, 0.0013 mol), copper (I)-iodide (0.063 g, 0.00033 mol), N,N′-dimethylethylenediamine (0.07 mL, 0.00066 mol) and potassium carbonate (0.32 g, 0.002 mol) were suspended in 1,4-dioxane (25 mL) priorly purged with N2 for 30 minutes. The reaction mixture was heated at 100 °C in an oil-bath for 24 h. After completion of the reaction by TLC (R_{f} 0.4, 7% MeOH in DCM) the reaction mixture was concentrated under reduced pressure. The residue obtained was purified by column chromatography (100-200 silica gel, 0-3% MeOH in DCM as eluent) to afford the title compound as yellow solid (65 mg, 23%).

¹H NMR (400 MHz, Trifluoroacetic acid-D) δ 7.53 (m, 2H), 7.31 (m, 2H), 7.09 m, 2H), 4.95 (m, 1H), 4.30 (m, 1H), 3.95 (m, 1H), 3.55 (d, J=76.8 Hz, 3H), 3.36 (m, 1H), 2.93 (m, 1H), 2.35 (d, J=57.2 Hz, 3H); MS(ESI): m/z 427.0 (M+H)⁺.

### Example 13

### 2-methyl-6-(2-oxoimidazolidin-1-yl)-4-(trifluoromethyl)phenyl(4-fluorophenyl)(methyl)carbamate

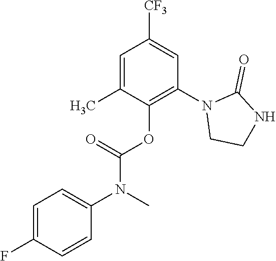 [SMILES: Cc1cc(C(F)(F)F)cc(N2CCNC2=O)c1OC(=O)N(C)c1ccc(F)cc1]

### Step 1: Synthesis of 4,4,5,5-tetramethyl-2-(2-methyl-4-(trifluoromethyl)phenyl)-1,3,2-dioxaborolane

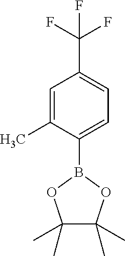

A solution of 1-bromo-2-methyl-4-(trifluoromethyl)benzene (1 g, 0.0042 mol) in 1,4 dioxane (20 mL) was purged with N2 in a sealed tube for 30 min and then added bispinacolato diboron (2.12 g, 0.00837 mol), potassium acetate (0.82 g, 0.00836 mol) and Pd(dppf)Cl₂·DCM (0.34 g, 0.00042 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with 5×50 mL n-hexane; washings collected was concentrated under reduced pressure to afford the yellow sticky solid (crude 3 g, Quantitative). “The product was taken as such to next step without further purification.

### Step 2: Synthesis of 2-methyl-4-(trifluoromethyl)phenol

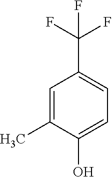

To a cold (0 °C) solution of 4,4,5,5-tetramethyl-2-(2-methyl-4-(trifluoromethyl)phenyl)-1,3,2-dioxaborolane (crude 6 g) in EtOH (50 mL), added hydrogen peroxide 30% aq. solution (4.1 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×30 mL). The organic layer collected was washed with water followed by brine, dried over anhydrous Na₂SO₄, filtered and concentrated under reduced pressure to afford the crude. The crude obtained was purified by column chromatography (100-200 silica gel, 10-15% EtOAc in Hexane as eluent) to afford the title compound as pale yellow liquid (0.7 g, 95% yield).

### Step 3: Synthesis of 2-iodo-6-methyl-4-(trifluoromethyl)phenol

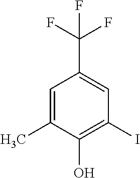

A solution of 2-methyl-4-(trifluoromethyl)phenol (0.7 g, 0.00397 mol) in THF:H₂O (3:1, 19 mL) was cooled 0 °C in an ice bath with stirring. After 15 minutes, was added 12 (1.1 g, 0.00433 mol) followed by Na₂CO₃ (0.46 g, 0.00433 mol) under inert atmosphere with stirring. Allowed the reaction mixture to stir at ambient temperature for 24 h. After completion of the reaction (TLC: R_{f} −0.35, 10% EtOAc in Hexane), the reaction mixture was cooled to 0 °C and then quenched with aq. sodium metabisulfite solution followed by extraction with EtOAc (2×50 mL). The organic layer collected was washed with water followed by brine, dried over anhydrous Na₂SO₄, filtered and concentrated under reduced pressure to afford the crude. The crude obtained was purified by column chromatography (100-200 silica gel, 0-5% EtOAc in Hexane as eluent) to afford the title compound as pale yellow liquid (1.0 g, 83% yield).

### Step 4: Synthesis of (4-fluorophenyl)(methyl)carbamic chloride

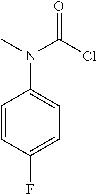

To a cold (0 °C) solution of triphosgene (0.57 g, 0.002 mol) in DCM (15 mL) added a solution of N-methyl-4-fluoro aniline (0.5 g, 0.0039 mol) and pyridine (0.61 g, 0.62 mL, 0.0078 mol) dropwise for over a period of 10 min. After that continued stirring at RT for 16 h. Progress of the reaction was monitored by TLC. After completion of the reaction, quenched the reaction mixture with 1M aq·HCl (15 mL) and then extracted with DCM (2×40 mL). The DCM layer separated was dried over anhydrous Na₂SO₄, filtered, and concentrated under reduced pressure to afford the title compound as green solid (0.7 g, 93%).

### Step 5: Synthesis of 2-iodo-6-methyl-4-(trifluoromethyl)phenyl (4-fluorophenyl)(methyl)carbamate

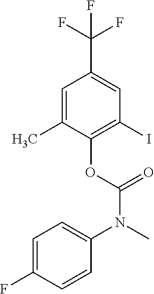 [SMILES: Cc1cc(C(F)(F)F)cc(I)c1OC(=O)N(C)c1ccc(F)cc1]

To a stirred solution of 2-iodo-6-methyl-4-(trifluoromethyl)phenol,5-difluorophenol (1 g, 0.0033 mol) in DMF (15 mL) at RT added K₂CO₃ (0.91 g, 0.0066 mol) and continued stirring at RT for 30 minutes. To the above mixture added a solution of (4-fluorophenyl)(methyl)carbamic chloride (0.62 g, 0.0033 mol) in DCM (15 mL) dropwise. The resultant reaction mixture was further stirred at RT for 7 h. After completion of the reaction was confirmed by TLC (R_{f} −0.25, 5% EtOAc in Hexane) quenched the reaction mixture with cold water (15 mL) and extracted with EtOAc (2×40 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off white solid (0.8 g, 53%).

### Step 6: Synthesis of 2-methyl-6-(2-oxoimidazolidin-1-yl)-4-(trifluoromethyl)phenyl (4-fluorophenyl)(methyl)carbamate

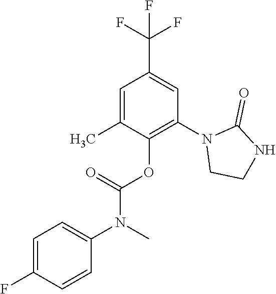 [SMILES: Cc1cc(C(F)(F)F)cc(N2CCNC2=O)c1OC(=O)N(C)c1ccc(F)cc1]

2-iodo-6-methyl-4-(trifluoromethyl)phenyl (4-fluorophenyl)(methyl)carbamate (0.3 g, 0.000662 mol), 2-imidazilidinone (0.114 g, 0.0013 mol), copper (I)-iodide (0.063 g, 0.00033 mol), N,N′-dimethylethylenediamine (0.07 mL, 0.00079 mol) and potassium carbonate (0.27 g, 0.002 mol) were suspended in 1,4-dioxane (25 mL) priorly purged with N2 for 30 minutes. The reaction mixture was heated at 100 °C in an oil-bath for 48 h. After completion of the reaction by TLC (R_{f} 0.4, 7% MeOH in DCM) the reaction mixture was concentrated under reduced pressure. The residue obtained was purified by column chromatography (100-200 silica gel, 0-3% MeOH in DCM as eluent) to afford the title compound as off-white solid (25 mg, 9%).

¹H NMR (400 MHz, Trifluoroacetic acid-D) δ 7.50 (m, 2H), 7.29 (m, 2H), 7.07 (dd, J=15.3 Hz, J=8.2 Hz 2H), 4.07 (m, 1H), 3.92 (m, 1H), 3.81 (m, 2H), 3.47 (d, J=64.6 Hz, 3H), 2.33 (d, J=42.2 Hz, 3H); MS(ESI): m/z 412.05 (M+H)⁺.

### Example 14

### 2-methyl-6-(2-oxoimidazolidin-1-yl)-4-(trifluoromethyl)phenyl (3-chloro-4-fluorophenyl)(methyl)carbamate

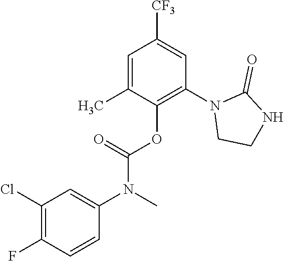 [SMILES: Cc1cc(C(F)(F)F)cc(N2CCNC2=O)c1OC(=O)N(C)c1ccc(F)c(Cl)c1]

### Step 1: Synthesis of 4,4,5,5-tetramethyl-2-(2-methyl-4-(trifluoromethyl)phenyl)-1,3,2-dioxaborolane

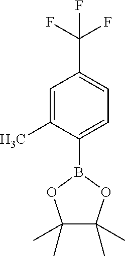

A solution of 1-bromo-2-methyl-4-(trifluoromethyl)benzene (1 g, 0.0041 mol) in 1,4 dioxane (20 mL) was purged with N2 in a sealed tube for 30 min and then added bispinacolato diboron (2.12 g, 0.00837 mol), potassium acetate (0.82 g, 0.00836 mol) and Pd(dppf)Cl₂·DCM (0.34 g, 0.00042 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (5×50 mL); washings collected was concentrated under reduced pressure to afford the yellow sticky solid (crude 3 g, Quantitative). The product was taken as such to next step without further purification.

### Step 2: Synthesis of 2-methyl-4-(trifluoromethyl)phenol

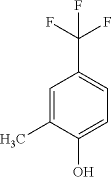

To a cold (0 °C) solution of 4,4,5,5-tetramethyl-2-(2-methyl-4-(trifluoromethyl)phenyl)-1,3,2-dioxaborolane (crude 6 g) in EtOH (50 mL), added hydrogen peroxide 30% aq. solution (4.1 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×30 mL). The organic layer collected was washed with water followed by brine, dried over anhydrous Na₂SO₄, filtered and concentrated under reduced pressure to afford the crude. The crude obtained was purified by column chromatography (100-200 silica gel, 10-15% EtOAc in Hexane as eluent) to afford the title compound as pale yellow liquid (0.7 g, 95% yield).

### Step 3: Synthesis of 2-iodo-6-methyl-4-(trifluoromethyl)phenol

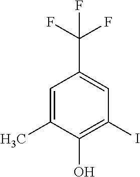

A solution of 2-methyl-4-(trifluoromethyl)phenol (0.05 g, 0.00028 mol) in THF:H₂O (3:1, 3 mL) was cooled 0 °C in an ice bath with stirring. After 15 minutes, was added I₂ (0.078 g, 0.00030 mol) followed by Na₂CO₃ (0.032 g, 0.00030 mol) under inert atmosphere with stirring. Allowed the reaction mixture to stir at ambient temperature for 16 h. After completion of the reaction (TLC: R⁸-0.5, 10% EtOAc in Hexane), the reaction mixture was cooled to 0 °C and then quenched with aq. sodium metabisulfite solution followed by extraction with EtOAc (2×30 mL). The organic layer collected was washed with water followed by brine, dried over anhydrous Na₂SO₄, filtered and concentrated under reduced pressure to afford the crude. The crude obtained was purified by column chromatography (100-200 silica gel, 10% EtOAc in Hexane as eluent) to afford the title compound as pale yellow solid (0.015 g, 17% yield).

### Step 4: Synthesis of (3-chloro-4-fluorophenyl)(methyl)carbamic chloride

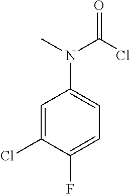

To a cold (0 °C) solution of triphosgene (0.091 g, 0.00031 mol) in DCM (3 mL) added a solution of 3-chloro-4-fluoro-N-methylaniline (0.1 g, 0.0006 mol) and pyridine (0.07 g, 0.08 mL, 0.0012 mol) dropwise for over a period of 10 min. After that continued stirring at RT for 16 h. Progress of the reaction was monitored by TLC. After completion of the reaction, quenched the reaction mixture with 1M aq·HCl (5 mL) and then extracted with DCM (2×30 mL). The DCM layer separated was dried over anhydrous Na₂SO₄, filtered, and concentrated under reduced pressure to afford the title compound as green solid (0.08 g, 58%).

### Step 5: Synthesis of 2-iodo-6-methyl-4-(trifluoromethyl)phenyl (3-chloro-4-fluorophenyl)(methyl) carbamate

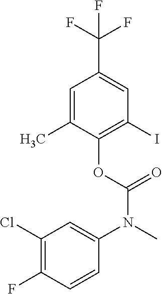 [SMILES: Cc1cc(C(F)(F)F)cc(I)c1OC(=O)N(C)c1ccc(F)c(Cl)c1]

To a stirred solution of 2-iodo-6-methyl-4-(trifluoromethyl)phenol, (0.1 g, 0.00033 mol) in DMF:DCM (1:1, 4 mL) at RT added K₂CO₃ (0.091 g, 0.00066 mol) and (3-chloro-4-fluorophenyl)(methyl)carbamic chloride (0.07 g, 0.00033 mol) and continued stirring at RT for 7 hours. After completion of the reaction was confirmed by TLC (R_{f} −0.2, 10% EtOAc in Hexane) quenched the reaction mixture with ice cold water (20 mL) and extracted with EtOAc (2×30 mL). The organic layer separated was combined, dried over anhydrous Na₂SO₄, filtered, and concentrated under reduced pressure to afford the crude. The crude obtained was purified by silica gel chromatography (100-200 and 2-4% EtOAc in Hexane as eluent) to afford the title compound as pale yellow liquid (0.1 g, 62%).

### Step 6: Synthesis of 2-methyl-6-(2-oxoimidazolidin-1-yl)-4-(trifluoromethyl)phenyl (3-chloro-4-fluorophenyl)(methyl)carbamate

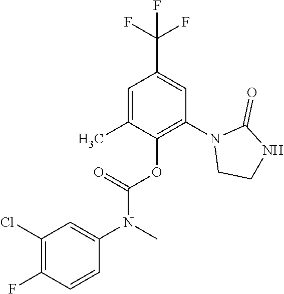 [SMILES: Cc1cc(C(F)(F)F)cc(N2CCNC2=O)c1OC(=O)N(C)c1ccc(F)c(Cl)c1]

To a stirred solution of 2-iodo-6-methyl-4-(trifluoromethyl)phenyl (3-chloro-4-fluorophenyl)(methyl) carbamate (0.1 g, 0.0002 mol), imidazolidin-2-one (0.035 g, 0.0004 mol), potassium carbonate (0.055 g, 0.0004 mol) and caesium fluoride (0.06 g, 0.0004 mol), were suspended in 1,4-dioxane (8 mL) priorly purged with N2 for 20 minutes, then copper (I)-iodide (0.02 g, 0.00010 mol), N,N′-dimethylethylenediamine (0.017 g, 0.020 mL, 0.00026 mol) were added. The reaction mixture was heated at 90 °C in an oil-bath for 24 h. After completion of the reaction by TLC (R_{f} 0.4, 5% MeOH in DCM) the reaction mixture was concentrated under reduced pressure. The residue obtained was purified by column chromatography (100-200 silica gel, 1-2% MeOH in DCM as eluent) to afford title compound as off-white solid (15 mg, 17%).

¹H NMR (500 MHz, Trifluoroacetic acid-D) δ 7.46 (m, 3H), 7.18 (m, 2H), 3.93 (m, 4H), 3.54 (s, 1H), 3.37 (s, 2H), 2.31 (d, J=48.7 Hz, 3H); MS(ESI): m/z 446.05 (M+H)⁺.

### Example 15

### 4-(trifluoromethyl)-2-methyl-6-(2-oxooxazolidin-3-yl)phenyl 4-fluorophenylmethylcarbamate

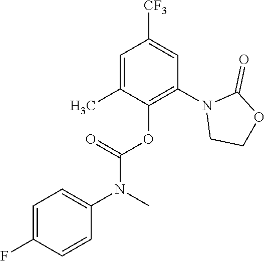 [SMILES: Cc1cc(C(F)(F)F)cc(N2CCOC2=O)c1OC(=O)N(C)c1ccc(F)cc1]

### Step 1: Synthesis of 4,4,5,5-tetramethyl-2-(2-methyl-4-(trifluoromethyl)phenyl)-1,3,2-dioxaborolane

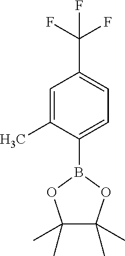

A solution of 1-bromo-2-methyl-4-(trifluoromethyl)benzene (1 g, 0.0041 mol) in 1,4 dioxane (20 mL) was purged with N2 in a sealed tube for 30 min and then added bispinacolato diboron (2.12 g, 0.00837 mol), potassium acetate (0.82 g, 0.00836 mol) and Pd(dppf)Cl₂·DCM (0.34 g, 0.00042 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (5×50 mL); washings collected was concentrated under reduced pressure to afford the yellow sticky solid (crude 3 g, Quantitative). The product was taken as such to next step without further purification.

### Step 2: Synthesis of 2-methyl-4-(trifluoromethyl)phenol

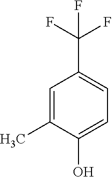

To a cold (0 °C) solution of 4,4,5,5-tetramethyl-2-(2-methyl-4-(trifluoromethyl)phenyl)-1,3,2-dioxaborolane (crude 6 g) in EtOH (50 mL), added hydrogen peroxide 30% aq. solution (4.1 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×30 mL). The organic layer collected was washed with water followed by brine, dried over anhydrous Na₂SO₄, filtered and concentrated under reduced pressure to afford the crude. The crude obtained was purified by column chromatography (100-200 silica gel, 10-15% EtOAc in Hexane as eluent) to afford the title compound as pale yellow liquid (0.7 g, 95% yield).

### Step 3: Synthesis of 2-iodo-6-methyl-4-(trifluoromethyl)phenol

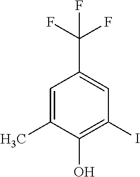

A solution of 2-methyl-4-(trifluoromethyl)phenol (0.05 g, 0.00028 mol) in THF:H₂O (3:1, 3 mL) was cooled 0 °C in an ice bath with stirring. After 15 minutes, was added 12 (0.078 g, 0.00030 mol) followed by Na₂CO₃ (0.68 g, 0.00030 mol) under inert atmosphere with stirring. Allowed the reaction mixture to stir at ambient temperature for 16 h. After completion of the reaction (TLC: R_{f} −0.5, 10% EtOAc in Hexane), the reaction mixture was cooled to 0 °C and then quenched with aq. sodium metabisulfite solution followed by extraction with EtOAc (2×30 mL). The organic layer collected was washed with water followed by brine, dried over anhydrous Na₂SO₄, filtered and concentrated under reduced pressure to afford the crude. The crude obtained was purified by column chromatography (100-200 silica gel, 10% EtOAc in Hexane as eluent) to afford the title compound as pale yellow solid (0.015 g, 17% yield).

### Step 4: Synthesis of (4-fluorophenyl)(methyl)carbamic chloride

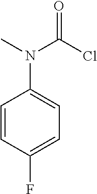

To a cold (0 °C) solution of triphosgene (0.57 g, 0.002 mol) in DCM (15 mL) added a solution of N-methyl-4-fluoro aniline (0.5 g, 0.0039 mol) and pyridine (0.61 g, 0.62 mL, 0.0078 mol) dropwise for over a period of 10 min. After that continued stirring at RT for 16 h. Progress of the reaction was monitored by TLC. After completion of the reaction, quenched the reaction mixture with 1M aq·HCl (15 mL) and then extracted with DCM (2×40 mL). The DCM layer separated was dried over anhydrous Na₂SO₄, filtered, and concentrated under reduced pressure to afford the title compound as green solid (0.7 g, 93%).

### Step 5: Synthesis of 2-iodo-6-methyl-4-(trifluoromethyl)phenyl (4-fluorophenyl)(methyl)carbamate

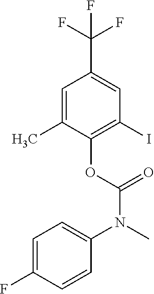 [SMILES: Cc1cc(C(F)(F)F)cc(I)c1OC(=O)N(C)c1ccc(F)cc1]

To a stirred solution of 2-iodo-6-methyl-4-(trifluoromethyl)phenol, (1.0 g, 0.0033 mol) in a mixture of DMF:DCM (1:1, 30 mL) at RT added K₂CO₃ (0.91 g, 0.0066 mol) and (4-fluorophenyl)(methyl)carbamic chloride (0.62 g, 0.0033 mol) and continued stirring at RT for 7 hours. After completion of the reaction was confirmed by TLC (R_{f} −0.25, 5% EtOAc in Hexane) quenched the reaction mixture with ice cold water (50 mL) and extracted with EtOAc (2×40 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off-white solid (0.8 g, 53%).

### Step 6: Synthesis of 2-methyl-6-(2-oxooxazolidin-3-yl)-4-(trifluoromethyl)phenyl(4-fluorophenyl) (methyl)carbamate

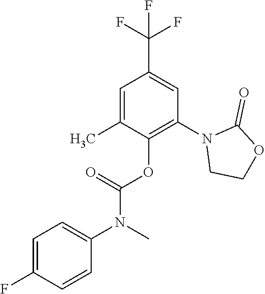 [SMILES: Cc1cc(C(F)(F)F)cc(N2CCOC2=O)c1OC(=O)N(C)c1ccc(F)cc1]

To a stirred solution of 2-iodo-6-methyl-4-(trifluoromethyl)phenyl (4-fluorophenyl) (methyl) carbamate (0.1 g, 0.00022 mol), oxazolidin-2-one (0.0383 g, 0.00044 mol), potassium carbonate (0.91 g, 0.00066 mol) and caesium fluoride (0.066 g, 0.00044 mol), were suspended in 1,4-dioxane (8 mL) priorly purged with N2 for 20 minutes, then copper (I)-iodide (0.02 g, 0.00010 mol), N,N′-dimethylethylenediamine (0.022 g, 0.027 mL, 0.00026 mol) were added. The reaction mixture was heated at 100 °C in an oil-bath for 24 h. After completion of the reaction by TLC (R_{f} 0.5, 60% EtOAc in Hexane) the reaction mixture was concentrated under reduced pressure. The residue obtained was purified by column chromatography (100-200 silica gel, 30-35% EtOAc in Hexane as eluent) to afford title compound as off-white solid (20 mg, 22%).

¹H NMR (500 MHz, Trifluoroacetic acid-D) δ 7.54 (dd, J=36.8 Hz, J=31.3 Hz, 2H), 7.31 (dq J=31.7 Hz, J=4.4 Hz, 2H), 7.10 (m, 2H), 4.68 (td, J=16.5 Hz, J=8.3 Hz, 2H), 4.11 (m, 2H), 3.48 (d, J=84.7 Hz, 2H), 2.35 (d, J=62.7 Hz, 3H); MS(ESI): m/z 412.0 (M+H)⁺.

### Example 16

### 2-(4,5-dihydroxy-2-oxoimidazolidin-1-yl)-4,6-bis(trifluoromethyl)phenyl N-(4-fluorophenyl)-N-methylcarbamate

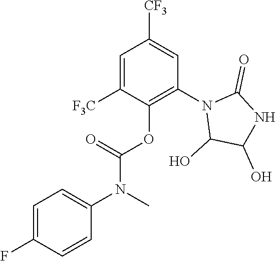 [SMILES: CN(C(=O)Oc1c(N2C(=O)NC(O)C2O)cc(C(F)(F)F)cc1C(F)(F)F)c1ccc(F)cc1]

### Step 1: Synthesis of 1-(2-hydroxy-3,5-bis(trifluoromethyl)phenyl)-1,3-dihydro-2H-imidazol-2-one

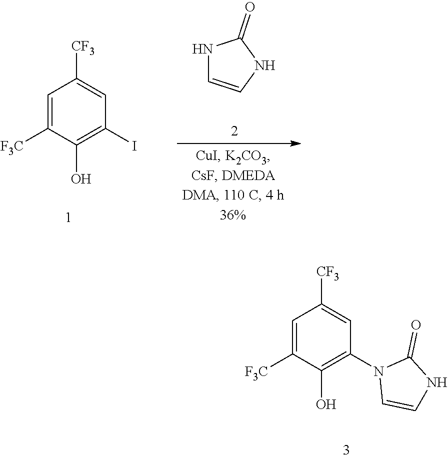

To a solution of compound 1 (1.0 g, 2.81 mmol, 1.0 eq) and compound 2 (474 mg, 5.63 mmol, 2.0 eq) in DMA (10 mL) at rt was added CuI (270 mg, 1.4 mmol, 0.5 eq), DMEDA (249 mg, 2.81 mmol, 1.0 eq), K₂CO₃ (782 mg, 5.62 mmol, 2.0 eq), CsF (855 mg, 5.62 mmol, 2.0 eq). The reaction mixture was heated at 110 °C in an oil-bath for 4 h. After completion of the reaction monitored by TLC (R_{f} 0.3, PE:EA=2:1) and LC-MS, water (20 mL) was added. The mixture was adjusted to pH 6 with HCl (2 N).

The mixture was extracted with ethyl acetate (30 mL×2). The combined organic layers were washed with brine, dried over Na₂SO₄ and filtered. The filtrate was concentrated under reduced pressure. The residue was purified by column chromatography (100-200 silica gel, 50% EA in PE) to afford compound 3 (320 mg, 36%) as pale-yellow solid.

### Step 2: Synthesis of tert-butyl 3-(2-hydroxy-3,5-bis(trifluoromethyl)phenyl)-2-oxo-2,3-dihydro-1H-imidazole-1-carboxylate

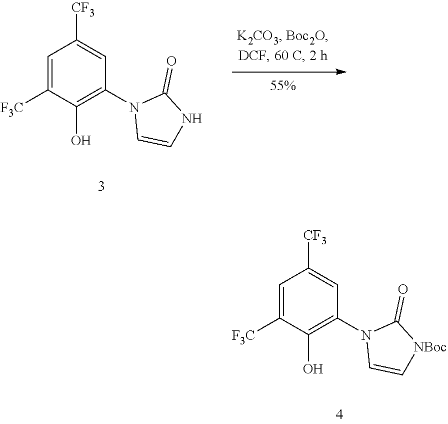

To a solution of compound 3 (150 mg, 0.48 mmol, 1.0 eq) and Boc₂O (125 mg, 0.58 mmol, 1.2 eq) in DMF (10 mL) at rt was added K₂CO₃ (167 mg, 1.2 mmol, 2.5 eq). The reaction mixture was heated at 60 °C in an oil-bath for 2 h. The mixture was extracted with ethyl acetate (30 mL×2). The combined organic layer was washed with brine, dried over Na₂SO₄ and filtered. The solution was concentrated under reduced pressure to give a residue. The residue was purified by column chromatography (100-200 silica gel, 20-50% EA in PE) to afford compound 4 (110 mg, 55%) as pale-yellow solid.

### Step 3: Synthesis of tert-butyl 3-(2-(((4-fluorophenyl)(methyl)carbamoyl)oxy)-3,5-bis(trifluoromethyl)phenyl)-2-oxo-2,3-dihydro-1H-imidazole-1-carboxylate

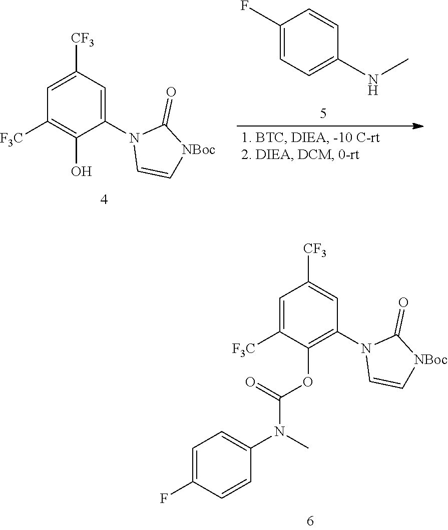

To a solution of compound 4 (110 mg, 0.35 mmol, 1.0 eq) in DCM (10 mL) at −10 °C was added DIEA (60 mg, 0.467 mmol, 1.5 eq) and triphosgene (117 mg, 0.395 mmol, 1.1 eq, in DCM (2 mL)). The reaction mixture was stirred at rt under N2 for 1 h. The reaction solution was concentrated under reduced pressure to remove the solvent. Then DCM (3 mL) was added to above residue. The solution was used for next step.

To a solution of compound 5 (85 mg, 0.52 mmol, 1.5 eq) and DIEA (68 mg, 0.52 mmol, 1.5 eq) in DCM (5 mL) at 0 °C was added the above solution. The mixture was stirred at rt under N2 for 1 h. After completion of the reaction checked by TLC (R_{f} 0.5, PE:EA=2:1) and LC-MS, HCl (1 N, 15 mL) was added. Then the mixture was extracted with DCM (20 mL×2). The combined organic layer was washed with brine, dried over Na₂SO₄ and filtered. The filtrate was concentrated under reduced pressure. The residue was purified by column chromatography (100-200 silica gel, 50% EA in PE as eluent) to give compound 6 (80 mg, 53%) as off-white solid.

### Step 4: Synthesis of AB25934

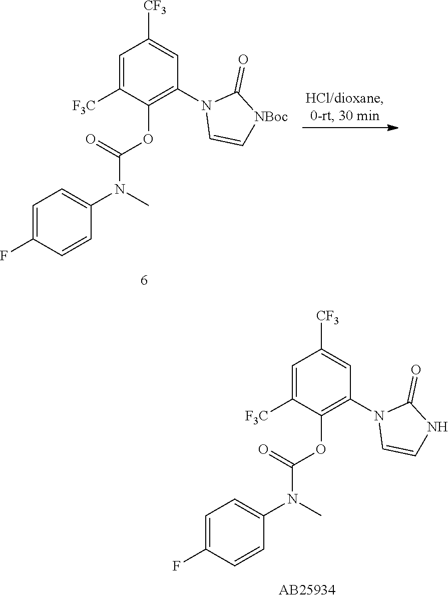 [SMILES: CN(C(=O)Oc1c(-n2ccn(C(=O)OC(C)(C)C)c2=O)cc(C(F)(F)F)cc1C(F)(F)F)c1ccc(F)cc1 ; CN(C(=O)Oc1c(-n2cc[nH]c2=O)cc(C(F)(F)F)cc1C(F)(F)F)c1ccc(F)cc1]

To a solution of HCl in dioxane (3 mL, 4 N) at rt was added compound 6 (80 mg, 0.14 mmol, 1.0 eq). The mixture was stirred for 30 min at rt under N2. After completion of the reaction by TLC (R_{f} 0.3, PE:EA=1:1), Na₂CO₃(aq) was added to adjust the solution to pH 8-9. The mixture was extracted with ethyl acetate (30 mL×2). The combined organic layers were washed with brine, dried over Na₂SO₄ and filtered. The filtration was concentrated under reduced pressure. The residue was purified by column chromatography (100-200 silica gel, 50% ethyl acetate in PE as eluent) to afford title compound (AB25934; 28 mg, 42%) as pale-yellow solid.

¹H NMR (400 MHz, CD₃OD): δ 8.15-8.18 (m, 1H), 7.99-8.05 (m, 1H), 7.27-7.32 (m, 2H), 7.10-7.16 (m, 2H), 6.69 (s, 0.38H) 6.66 (s, 1H), 6.64 (s, 0.57H), 3.46 (s, 1H), 3.22 (s, 2H); LC-MS: 464.00 [M+1]⁺.

### Step 5: Synthesis of 2-(4,5-dihydroxy-2-oxoimidazolidin-1-yl)-4,6-bis(trifluoromethyl)phenyl N-(4-fluorophenyl)-N-methylcarbamate

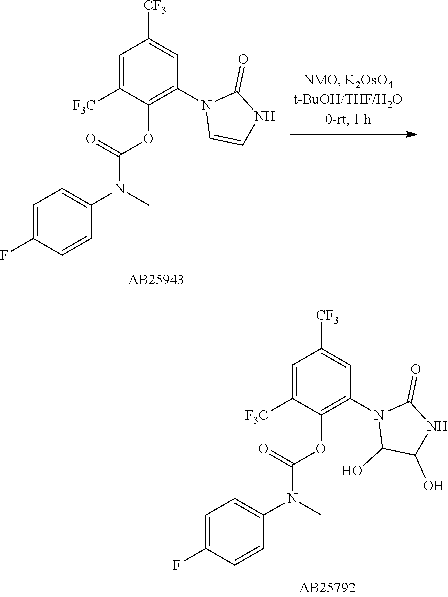 [SMILES: CN(C(=O)Oc1c(-n2cc[nH]c2=O)cc(C(F)(F)F)cc1C(F)(F)F)c1ccc(F)cc1 ; CN(C(=O)Oc1c(N2C(=O)NC(O)C2O)cc(C(F)(F)F)cc1C(F)(F)F)c1ccc(F)cc1]

To a solution of AB25943 (AB25943, 60 mg, 0.12 mmol, 1.0 eq) and in t-BuOH/THF/H₂O (2 mL/2 mL/1 mL) was added NMO (20 mg, 0.16 mmol, 1.4 eq), K₂OsO₄ (0.7 mg, 0.0024 mmol, 0.02 eq) at 0 °C. The reaction mixture was stirred at rt for 1 h. After completion of the reaction monitored by TLC (R_{f} 0.1, PE:EA=1:1) and LC-MS, the mixture was extracted with ethyl acetate (30 mL×2). The combined organic layers were washed with brine, dried with Na₂SO₄ and filtered. The filtrate was concentrated under reduced pressure. The residue was purified by column chromatography (100-200 silica gel, 70% ethyl acetate in PE as eluent) to afford title compound (AB25792; 22.3 mg, 34%) as white solid.

¹H NMR (400 MHz, CD₃OD): δ 7.88-7.99 (m, 1H), 7.81 (s, 1H), 7.27-7.32 (m, 1H), 7.30-7.42 (m, 1H), 7.11-7.05 (m, 2H) 5.12 (m, 1H), 4.94 (s, 1H), 3.47 (s, 1H), 3.32 (s, 2H); LCMS: 495.95 [M−1]⁻.

### Example 17

### 2-[(2R)-2-methyl-5-oxopyrrolidin-1-yl]-4,6-bis(trifluoromethyl)phenyl N-(4-fluorophenyl)-N-methylcarbamate

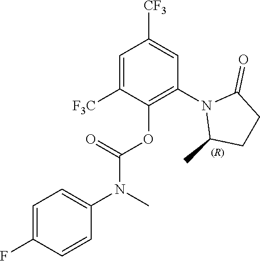

### Step 1: Synthesis of 2-(2,4-bis(trifluoromethyl)phenyl)-4,4,5,5-tetramethyl-1,3,2-dioxaborolane

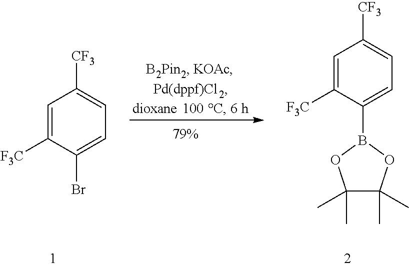

A solution of 1-bromo-2,4-bis(trifluoromethyl)benzene (25.0 g, 85.3 mmol, 1.0 eq) in dioxane (300 mL) was purged with N2 in a sealed tube for 2 min and then added bis(pinacolato)diboron (43.4 g, 170 mmol, 2.0 eq), potassium acetate (16.8 g, 170 mmol) and Pd(dppf)Cl₂-DCM (2.4 g, 8.53 mmol, 0.1 eq) with stirring under inert atmosphere. The tightly closed sealed tube was heated at 100 °C in an oil bath for 16 h with stirring. Progress of the reaction was monitored by TLC (Rf=0.5, 10% EtOAc in Hexane). After completion of the reaction, the reaction mixture was concentrated under reduced pressure. The residue was partitioned between ethyl acetate (200 mL) and water (150 mL). The organic layer was dried over sodium sulfate, and concentrated under reduced pressure. The residue was purified by silica gel chromatography (PE:EA=1:20) to give compound 2 (23.0 g, 79%).

### Step 2: Synthesis of 2,4-bis(trifluoromethyl)phenol

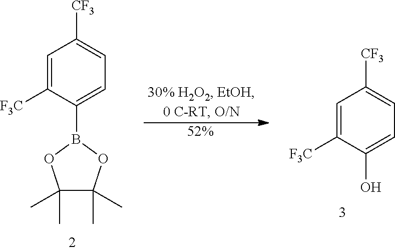 [SMILES: CC1(C)OB(c2ccc(C(F)(F)F)cc2C(F)(F)F)OC1(C)C ; "52%" ; "30% H2O2, EtOH,0 C-RT, O/N" ; Oc1ccc(C(F)(F)F)cc1C(F)(F)F]

To a solution of 2-(2,4-bis(trifluoromethyl)phenyl)-4,4,5,5-tetramethyl-1,3,2-dioxaborolane (23.0 g, 67.6 mmol, 1.0 eq) in ethanol (300 mL) at 0 °C was added hydrogen peroxide (30% aq. Solution, 24 mL) under inert atmosphere with stirring. The reaction mixture was stirred at ambient temperature for 16 h. After completion of the reaction confirmed by TLC (Rf=0.2, 10% EtOAc in Hexane), the reaction mixture was cooled to 0 °C and quenched with aq. sodium metabisulfite solution followed by extraction with EtOAc (2×100 mL). The organic layer collected was washed with water followed by brine, dried over anhydrous sodium sulfate, filtered and concentrated under reduced pressure to afford a crude mixture, which was purified by column chromatography (silica gel, 0-20% EtOAc in Hexane as eluent) to afford compound 3 (8.1, 52%) as pale yellow liquid.

### Step 3: Synthesis of 2-iodo-4,6-bis(trifluoromethyl)phenol

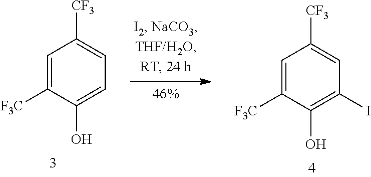 [SMILES: Oc1ccc(C(F)(F)F)cc1C(F)(F)F ; Oc1c(I)cc(C(F)(F)F)cc1C(F)(F)F]

A solution of 2,4-bis(trifluoromethyl)phenol (8.0 g, 34.8 mol) in THF:H₂O (3:1, 160 mL) was cooled 0 °C in an ice bath with stirring. After 15 minutes, iodine (10.6 g, 41.7 mmol) was added, followed by sodium carbonate (4.4 g, 41.5 mmol) under nitrogen with stirring. After being stirred at ambient temperature for 24 h, the reaction mixture was cooled to 0 °C and then quenched with aqueous sodium metabisulfite solution followed by extraction with ethyl acetate (2×150 mL). The organic layers were washed with water and brine, dried over anhydrous sodium sulfate, filtered and concentrated under reduced pressure. The crude mixture was purified by column chromatography (silica gel, 0-5% EtOAc in hexane) to afford compound 4 (5.8 g, 46%) as pale yellow solid.

### Step 4: Synthesis of (R)-1-(2-hydroxy-3,5-bis(trifluoromethyl)phenyl)-5-methylpyrrolidin-2-one

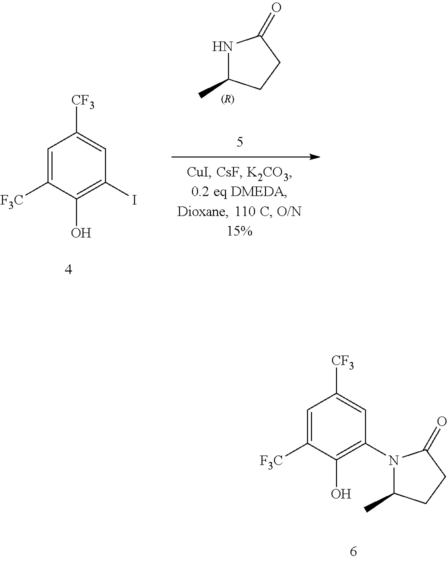

To a solution of compound 4 (356 mg, 1.0 mmol, 1.0 eq) and compound 5 (200 mg, 2 mmol, 2.0 eq) in dioxane (10 mL) at it was added CuI (95 mg, 0.5 mmol, 0.5 eq), CsF (304 mg, 2 mmol, 2.0 eq) and N,N′-dimethylethylenediamine (44 mg, 0.5 mmol, 0.5 eq). The reaction mixture was heated at 110 °C in an oil-bath for 24 h. After completion of the reaction monitored by TLC (R_{f} 0.3, PE:EA=1:1), water (20 mL) was added and adjusted to pH6 with HCl (2 N). The mixture was extracted with ethyl acetate (30 mL×2). The combined organic layers were washed with brine, dried with Na₂SO₄ and filtered. The filtration was concentrated under reduced pressure. The residue was purified by column chromatography (silica gel, 50% EA in PE as eluent) to afford compound 6 (50 mg, 15%) as pale-yellow solid.

### Step 5: Synthesis of AB25793

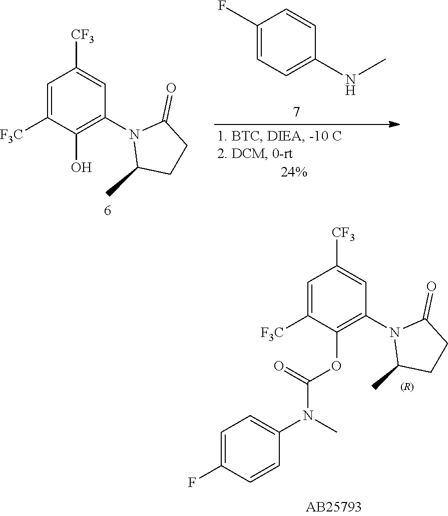

To a solution of compound 6 (30 mg, 0.092 mmol, 1.0 eq) in DCM (10 mL) under N2 at −10 °C was added DIEA (13 mg, 0.101 mmol, 1.1 eq), and triphosgene (24 mg, 0.092 mmol, 1.0 eq) in DCM (2 mL). The reaction mixture was stirred for 1 h at rt. The reaction solution was concentrated under reduced pressure to remove the solvent. Then DCM (3 mL) added to above reaction mixture, which was used for next step.

### Step 6: Synthesis of 2-[(2R)-2-methyl-5-oxopyrrolidin-1-yl]-4,6-bis(trifluoromethyl)phenyl N-(4-fluorophenyl)-N-methylcarbamate

To a solution of compound 7 (20 mg, 0.16 mmol, 1.7 eq) in DCM (5 mL) at 0 °C was added the solution above. The mixture was stirred at rt under N2 for 1 h. HCl (1 N, 15 mL) was added. The mixture was extracted with DCM (20 mL×2). The combined organic layers were washed with brine, dried with sodium sulfate and filtered. The filtration was concentrated under reduced pressure. The residue was purified by prep-TLC (silica gel) and prep-HPLC (C-18) to give title compound (AB25793; 10.8 mg, 24%) as off-white solid.

¹H NMR (400 MHz, CDCl₃): δ 8.00-7.95 (m, 2H), 7.37 (m, 2H), 7.17-7.15 (m, 2H), 4.33-4.16 (m, 1H), 3.47 (s, 1H) 3.47 (s, 2.09H), 2.54-2.45 (m, 2H), 2.44-2.41 (m, 1H), 1.82-1.80 (m, 1H), 1.16 (d, J=4 Hz, 1H) 1.01 (d, J=4 Hz, 2H); LC-MS: 479.10 [M+H]⁻.

### Example 18

### 2-(2-oxo-4-(trifluoromethyl)imidazolidin-1-yl)-4,6-bis(trifluoromethyl)phenyl (4-fluorophenyl)(methyl)carbamate

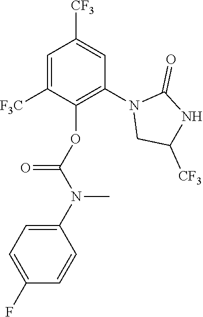 [SMILES: CN(C(=O)Oc1c(N2CC(C(F)(F)F)NC2=O)cc(C(F)(F)F)cc1C(F)(F)F)c1ccc(F)cc1]

### Step 1: Synthesis of 2-iodo-4,6-bis(trifluoromethyl)phenol

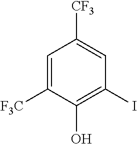 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20 mL) was cooled 0 °C in an ice bath with stirring. After 15 minutes, was added 12 (1.4 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 30% EtOAc in Hexane followed by 30% DCM in Hexane), the reaction mixture was cooled to 0 °C and then quenched with aq. sodium metabisulfite solution followed by extraction with EtOAc (2×100 mL). The organic layer collected was washed with water followed by brine, dried over anhydrous Na₂SO₄, filtered and concentrated under reduced pressure to afford the crude. The crude obtained was purified by column chromatography (100-200 silica gel, 5-10% EtOAc in Hexane as eluent) to afford the title compound as pale yellow solid (0.98 g, 64% yield).

### Step 2: Synthesis of 2-iodo-4,6-bis(trifluoromethyl)phenyl (4-fluorophenyl)(methyl)carbamate

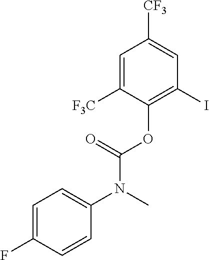 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80 °C for 4 hours. After completion of the reaction was confirmed by TLC (R_{f} −0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 0-10% EtOAc in Hexane as eluent) to afford the title compound as off off-white solid (0.66 g, 93%).

### Step 3: Synthesis of 2-(2-oxo-4-(trifluoromethyl)imidazolidin-1-yl)-4,6-bis(trifluoromethyl)phenyl (4-fluorophenyl)(methyl)carbamate

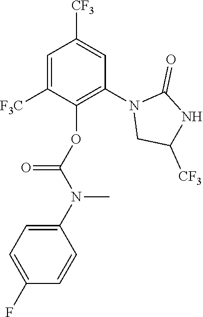 [SMILES: CN(C(=O)Oc1c(N2CC(C(F)(F)F)NC2=O)cc(C(F)(F)F)cc1C(F)(F)F)c1ccc(F)cc1]

2-iodo-4,6-bis(trifluoromethyl)phenyl (4-fluorophenyl)(methyl)carbamate (200 mg, 0.3944 mmol), 4-trifluoromethyl-2-imidazolidinone (109 mg, 0.7078 mmol), copper (1) iodide (37 mg, 0.1922 mmol), cesium fluoride (121 mg, 0.7990 mmol), N,N′-dimethylethylenediamine (43 uL, 0.3944 mmol) and anhydrous powered potassium carbonate (0.7282 mmol, 101 mg) were added to degassed anhydrous 1,4-dioxane (4.0 mL) under nitrogen. The resulting suspension was stirred at 90 °C overnight. The reaction was cooled to room temperature, diluted with methanol and filtered. The clear blue filtrate was concentrated down to yield a gray solid. This was suspended in dichloromethane, filtered through plug of celite and concentrated down. The crude product was purified by column chromatography on silica gel using a gradient solvent system of 0 to 10% MeOH in DCM to afford still impure product. It was repurified by column chromatography on silica gel using a gradient solvent system of 0 to 100% EtOAc in hexanes to afford pure titled compound as a yellow oil (21 mg, 10%).

¹H NMR (400 MHz, CDCl₃) δ 7.81-7.89 (2H), 7.28 (m, 2H), 7.09 (m, 2H), 5.37-5.44 (1H), 4.28 (bs, 1H), 4.05 (bs, 1H), 3.49 (m, 1H), 3.33 (m, 2H); ESIMS: m/z 534.1 [(M+H)⁺].

### Example 19

### 2,4-bis(trifluoromethyl)-6-((S)-3-hydroxy-2-oxopyrrolidin-1-yl)phenyl 4-fluorophenylmethylcarbamate

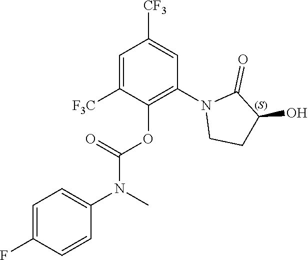

### Step 1: Synthesis of 2-(2,4-bis(trifluoromethyl)phenyl)-4,4,5,5-tetramethyl-1,3,2-dioxaborolane

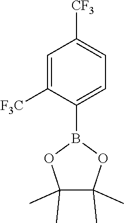 [SMILES: CC1(C)OB(c2ccc(C(F)(F)F)cc2C(F)(F)F)OC1(C)C]

A solution of 1-bromo-2,4-bis(trifluoromethyl)benzene (3 g, 0.0102 mol) in 1,4 dioxane (90 mL) was purged with N2 in a sealed tube for 30 min and then added bispinacolato diboron (2.12 g, 0.00837 mol), potassium acetate (0.82 g, 0.00836 mol) and Pd(dppf)Cl₂·DCM (0.34 g, 0.00042 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4×75 mL); washings collected was concentrated under reduced pressure to afford the yellow sticky solid (crude 6 g, Quantitative). The product was taken as such to next step without further purification.

### Step 2: Synthesis of 2,4-bis(trifluoromethyl)phenol

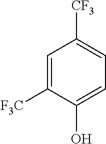 [SMILES: Oc1ccc(C(F)(F)F)cc1C(F)(F)F]

To a cold (0 °C) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×100 mL). The organic layer collected was washed with water followed by brine, dried over anhydrous Na₂SO₄, filtered and concentrated under reduced pressure to afford the crude. The crude obtained was purified by column chromatography (100-200 silica gel, 10-15% EtOAc in Hexane as eluent) to afford the title compound as pale yellow liquid (1.5 g, 65% yield).

### Step 3: Synthesis of 2-iodo-4,6-bis(trifluoromethyl)phenol

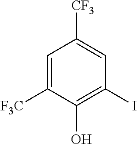 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20 mL) was cooled 0 °C in an ice bath with stirring. After 15 minutes, was added 12 (1.4 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 30% EtOAc in Hexane followed by 30% DCM in Hexane), the reaction mixture was cooled to 0 °C and then quenched with aq. sodium metabisulfite solution followed by extraction with EtOAc (2×100 mL). The organic layer collected was washed with water followed by brine, dried over anhydrous Na₂SO₄, filtered and concentrated under reduced pressure to afford the crude. The crude obtained was purified by column chromatography (100-200 silica gel, 5-10% EtOAc in Hexane as eluent) to afford the title compound as pale yellow solid (0.98 g, 64% yield).

### Step 4: Synthesis of (4-fluorophenyl)(methyl)carbamic chloride

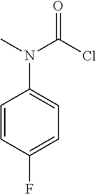

To a cold (0 °C) solution of triphosgene (1.6 g, 0.0128 mol) in DCM (40 mL) added a solution of N-methyl-4-fluoro aniline (1.89 g, 0.0063 mol) and pyridine (2.0 g, 2.0 mL, 0.025 mol) dropwise for over a period of 10 min. After that continued stirring at RT for 16 h. Progress of the reaction was monitored by TLC. After completion of the reaction, quenched the reaction mixture with 1M aq·HCl (50 mL) and then extracted with DCM (2×50 mL). The DCM layer separated was dried over anhydrous Na₂SO₄, filtered, and concentrated under reduced pressure to afford the title compound as green solid (1.8 g, 75%).

### Step 5: Synthesis of 2-iodo-4,6-bis(trifluoromethyl)phenyl (4-fluorophenyl)(methyl)carbamate

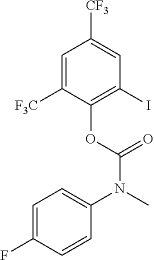 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80 °C for 4 hours. After completion of the reaction was confirmed by TLC (R_{f} −0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 0-10% EtOAc in Hexane as eluent) to afford the title compound as off off-white solid (0.66 g, 93%).

### Step 6: Synthesis of (S)-2-(3-hydroxy-2-oxopyrrolidin-1-yl)-4,6-bis(trifluoromethyl)phenyl (4-fluorophenyl)(methyl)carbamate

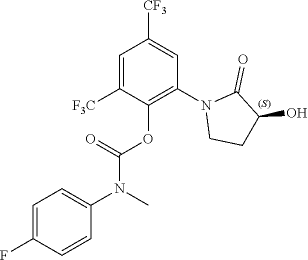

To a stirred solution of 2-iodo-4,6-bis(trifluoromethyl)phenyl (4-fluorophenyl)(methyl)carbamate (0.2 g, 0.00039 mol), (S)-3-hydroxypyrrolidin-2-one (0.079 g, 0.00078 mol), potassium carbonate (0.10 g, 0.00078 mol) and caesium fluoride (0.12 g, 0.00078 mol), were suspended in 1,4-dioxane (16 mL) priorly purged with N2 for 30 minutes, then copper (I)-iodide (0.037 g, 0.00019 mol), N,N′-dimethylethylenediamine (0.034 g, 0.42 mL, 0.00039 mol) were added. The reaction mixture was heated at 100 °C in an oil-bath for 24 h. After completion of the reaction by TLC (R_{f} 0.4, 5% MeOH in DCM) the reaction mixture was concentrated under reduced pressure. The residue obtained was purified by column chromatography (100-200 silica gel, 0-3% MeOH in DCM as eluent) to afford solid. The solid was further purified by preparative TLC using CH₂Cl₂/MeOH (98/2) as mobile phase to obtain the title compound as off-white solid (17 mg, 9%).

¹H NMR (500 MHz, DMSO-d₆) δ 8.26 (d, J=28.9 Hz, 1H), 8.08 (d, J=40.6 Hz, 1H), 7.44 (s, 2H), 7.26 (t, 2H), 4.32 (s, 1H), 3.80 (s, 1H), 3.81 (m, 1H), 3.39 (s, 2H), 3.25 (d, J=69.5 Hz, 3H), 1.95 (d, J=37.9 Hz, 1H); MS(ESI): m/z 481.25 (M+H)⁺.

### Example 20

### 2,4-bis(trifluoromethyl)-6-(3-(2-hydroxypropyl)-2-oxoimidazolidin-1-yl)phenyl 4-fluorophenylmethylcarbamate

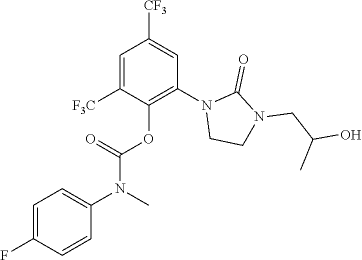 [SMILES: CC(O)CN1CCN(c2cc(C(F)(F)F)cc(C(F)(F)F)c2OC(=O)N(C)c2ccc(F)cc2)C1=O]

### Step 1: Synthesis of 2-(2,4-bis(trifluoromethyl)phenyl)-4,4,5,5-tetramethyl-1,3,2-dioxaborolane

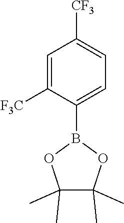 [SMILES: CC1(C)OB(c2ccc(C(F)(F)F)cc2C(F)(F)F)OC1(C)C]

A solution of 1-bromo-2,4-bis(trifluoromethyl)benzene (3 g, 0.01 mol) in 1,4 dioxane (90 mL) was purged with N2 in a sealed tube for 30 min and then added bispinacolato diboron (5.07 g, 0.02 mol), potassium acetate (1.96 g, 0.02 mol) and Pd(dppf)Cl₂·DCM (0.816 g, 0.001 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4×75 mL); washings collected was concentrated under reduced pressure to afford the yellow sticky solid (crude 6 g, Quantitative). The product was taken as such to next step without further purification.

### Step 2: Synthesis of 2,4-bis(trifluoromethyl)phenol

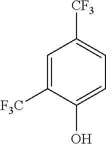 [SMILES: Oc1ccc(C(F)(F)F)cc1C(F)(F)F]

To a cold (0 °C) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×100 mL). The organic layer collected dried over anhydrous Na₂SO₄, concentrated under reduced pressure to afford the crude. The crude was purified by column chromatography (100-200 silica gel, 10-15% EtOAc in Hexane as eluent) to afford the title compound as pale yellow liquid (1.5 g, 65% yield).

### Step 3: Synthesis of 2-iodo-4,6-bis(trifluoromethyl)phenol

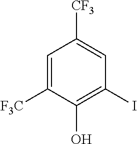 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20:6 mL) was cooled 0 °C in an ice bath with stirring. After 15 minutes, was added 12 (1.43 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 30% EtOAc in Hexane followed by 30% DCM in Hexane), the reaction mixture was cooled to 0 °C and then quenched with aq. sodium metabisulfite solution followed by extraction with EtOAc (2×100 mL). The organic layer collected dried over anhydrous Na₂SO₄, filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 4: Synthesis of (4-fluorophenyl)(methyl)carbamic chloride

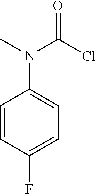

To a cold (0 °C) solution of triphosgene (10.6 g, 0.0128 mol) in DCM (40 mL) added a solution of N-methyl-4-fluoro aniline (1.89 g, 0.0064 mol) and pyridine (2.52 g, 2.6 mL, 0.032 mol) dropwise for over a period of 10 min. After that continued stirring at RT for 16 h. Progress of the reaction was monitored by TLC (R_{f} −0.7, 10% EtOAc in Hexane (×4)). After completion of the reaction, quenched the reaction mixture with 1M aq·HCl (50 mL) and then extracted with DCM (2×50 mL). The DCM layer separated was dried over anhydrous Na₂SO₄, filtered, and concentrated under reduced pressure to afford the title compound as green solid (1.8 g, 75%).

### Step 5: Synthesis of 2-iodo-4,6-bis(trifluoromethyl)phenyl (4-fluorophenyl)(methyl)carbamate

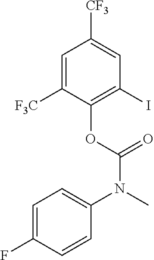 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80 °C for 4 hours. After completion of the reaction was confirmed by TLC (R_{f} −0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off-white solid (0.66 g, 93%).

### Step 6: Synthesis of 2-(2-oxoimidazolidin-1-yl)-4,6-bis(trifluoromethyl)phenyl (4-fluorophenyl)(methyl) carbamate

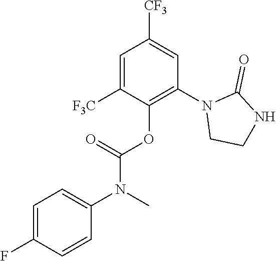 [SMILES: CN(C(=O)Oc1c(N2CCNC2=O)cc(C(F)(F)F)cc1C(F)(F)F)c1ccc(F)cc1]

2-iodo-4,6-bis(trifluoromethyl)phenyl (4-fluorophenyl)(methyl)carbamate (0.2 g, 0.00039 mol), 2-imidazilidinone (0.064 g, 0.0007 mol), copper (I)-iodide (0.037 g, 0.00019 mol), N,N′-dimethylethylenediamine (0.07 mL, 0.00039 mol), cesium fluoride (0.12 g, 0.00079 mol) and potassium carbonate (0.1 g, 0.00072 mol) were suspended in 1,4-dioxane priorly purged with N2 for 30 minutes (16 mL). The reaction mixture was heated at 90 °C in an oil-bath for 24 h. After completion of the reaction by TLC (R_{f} 0.4, 5% MeOH in DCM) the reaction mixture was concentrated under reduced pressure. The residue obtained was purified by column chromatography (100-200 silica gel, 0-3% MeOH in DCM as eluent) to afford the title compound as off-white solid (50 mg). The solid was re-purified by preparative TLC (solid phase: merck, 20×20 cm, silicagel 60 GF₂₅₄, 1 mm, PLC glass plate, 2% MeOH in DCM as eluent) to afford the title compound as white solid (28 mg, 15%).

### Step 7: Synthesis of 2-(2-oxo-3-(2-oxopropyl)imidazolidin-1-yl)-4,6-bis(trifluoromethyl)phenyl (4-fluorophenyl) (methyl)carbamate

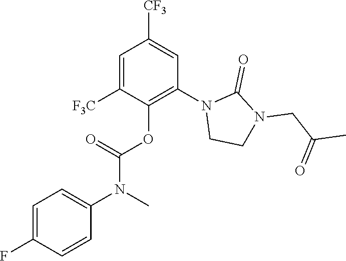 [SMILES: CC(=O)CN1CCN(c2cc(C(F)(F)F)cc(C(F)(F)F)c2OC(=O)N(C)c2ccc(F)cc2)C1=O]

A solution of 2-(2-oxoimidazolidin-1-yl)-4,6-bis(trifluoromethyl)phenyl (4-fluorophenyl) (methyl) carbamate (0.11 g, 0.0002 mol) in DMF in a sealed tube was purged with N2 for 10 min and then added cesium carbonate (0.23 g, 0.0007 mol), potassium iodide (0.066 g, 0.0004 mol) and 1-bromopropan-2-one (0.05 mL, 0.0004 mol). The sealed tube was heated at 80 °C for 24 h. After completion of the reaction confirmed by TLC (R_{f} −0.6, 2% MeOH/DCM), added cold water followed by extraction with ethyl acetate (2×50 mL). The organic layer separated and combined was dried over anhydrous Na₂SO₄, filtered, and concentrated under reduced pressure to afford the crude. The crude obtained was purified by preparative TLC (Solid phase: 20×20 cm, silicagel 60 GF₂₅₄, 1 mm, PLC Merck glass plates, 1% MeOH in DCM as eluent) to afford the title compound as sticky white solid (35 mg, 34%).

### Step 8: Synthesis of 2-(3-(2-hydroxypropyl)-2-oxoimidazolidin-1-yl)-4,6-bis(trifluoromethyl)phenyl (4-fluorophenyl)(methyl)carbamate

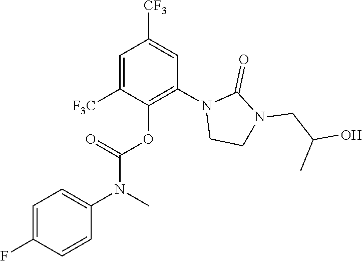 [SMILES: CC(O)CN1CCN(c2cc(C(F)(F)F)cc(C(F)(F)F)c2OC(=O)N(C)c2ccc(F)cc2)C1=O]

To a stirred solution of 2-(2-oxo-3-(2-oxopropyl)imidazolidin-1-yl)-4,6-bis(trifluoromethyl)phenyl (4-fluorophenyl) (methyl)carbamate (0.04 g, 0.000076 mol) in methanol cooled at 0 °C was added sodium borohydride (0.0058 g, 0.00015 mol) under N2 atmosphere and allowed the mixture to stir at 20 °C for 1 h. After completion of the reaction confirmed by TLC (R_{f} −0.3, 5% MeOH/DCM), concentrated the reaction mixture under reduced pressure to afford the crude. The crude was washed with water followed by extraction with ethyl acetate (2×20 mL). The organic layer separated and combined was dried over anhydrous Na₂SO₄, filtered, and concentrated under reduced pressure to afford the residue. The crude obtained was purified by preparative TLC (Solid phase: 20×20 cm, silica gel 60 GF₂₅₄, 1 mm, PLC Merck glass plates, 2% MeOH in DCM as eluent) to afford the title compound as white solid (17 mg, 45%).

¹H NMR (400 MHz, DMSO-d₆) δ 8.25 (d, J=17.7 Hz, 1H), 7.94 (d, J=28.6 Hz, 1H), 7.45 (s, 2H), 7.28 (m, 2H), 4.78 (s, 1H), 3.85 (m, 1H), 3.59 (d, J=4.4 Hz, 2H), 3.46 (m, 3H), 3.13 (d, J=26.1 Hz, 2H), 1.24 (s, 2H), 1.07 (d, J=4.8 Hz, 3H); MS(ESI): m/z 524.30 (M+H)⁺.

### Example 21

### 2-{2-oxo-3-[2-(sulfamoyloxy)ethyl]imidazolidin-1-yl}-4,6-bis(trifluoromethyl)phenyl N-(4-fluorophenyl)-N-methylcarbamate

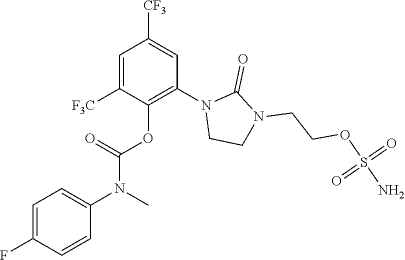 [SMILES: CN(C(=O)Oc1c(N2CCN(CCOS(N)(=O)=O)C2=O)cc(C(F)(F)F)cc1C(F)(F)F)c1ccc(F)cc1]

### Step 1: Synthesis of 2-(2,4-bis(trifluoromethyl)phenyl)-4,4,5,5-tetramethyl-1,3,2-dioxaborolane

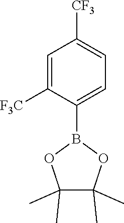 [SMILES: CC1(C)OB(c2ccc(C(F)(F)F)cc2C(F)(F)F)OC1(C)C]

A solution of 1-bromo-2,4-bis(trifluoromethyl)benzene (3 g, 0.01 mol) in 1,4 dioxane (90 mL) was purged with N2 in a sealed tube for 30 min and then added bispinacolato diboron (5.07 g, 0.02 mol), potassium acetate (1.96 g, 0.02 mol) and Pd(dppf)Cl₂·DCM (0.816 g, 0.001 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4×75 mL); washings collected was concentrated under reduced pressure to afford the yellow sticky solid (crude 6 g, Quantitative). The product was taken as such to next step without further purification.

### Step 2: Synthesis of 2,4-bis(trifluoromethyl)phenol

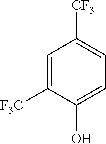 [SMILES: Oc1ccc(C(F)(F)F)cc1C(F)(F)F]

To a cold (0 °C) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×100 mL). The organic layer collected dried over anhydrous Na₂SO₄, concentrated under reduced pressure to afford the crude. The crude was purified by column chromatography (100-200 silica gel, 10-15% EtOAc in Hexane as eluent) to afford the title compound as pale yellow liquid (1.5 g, 65% yield).

### Step 3: Synthesis of 2-iodo-4,6-bis(trifluoromethyl)phenol

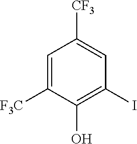 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20:6 mL) was cooled 0 °C in an ice bath with stirring. After 15 minutes, was added 12 (1.43 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 20% EtOAc in Hexane), the reaction mixture was cooled to 0 °C and then quenched with aq. sodium metabisulfite solution followed by extraction with EtOAc (2×100 mL). The organic layer collected dried over anhydrous Na₂SO₄, filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 4: Synthesis of (4-fluorophenyl)(methyl)carbamic chloride

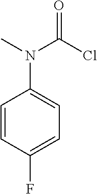

To a cold (0 °C) solution of triphosgene (10.6 g, 0.0128 mol) in DCM (40 mL) added a solution of N-methyl-4-fluoro aniline (1.89 g, 0.0064 mol) and pyridine (2.52 g, 2.6 mL, 0.032 mol) dropwise for over a period of 10 min. After that continued stirring at RT for 16 h. Progress of the reaction was monitored by TLC (R_{f} −0.7, 10% EtOAc in Hexane). After completion of the reaction, quenched the reaction mixture with 1M aq·HCl (50 mL) and then extracted with DCM (2×50 mL). The DCM layer separated was dried over anhydrous Na₂SO₄, filtered, and concentrated under reduced pressure to afford the title compound as green solid (1.8 g, 75%).

### Step 5: Synthesis of 2-iodo-4,6-bis(trifluoromethyl)phenyl (4-fluorophenyl)(methyl)carbamate

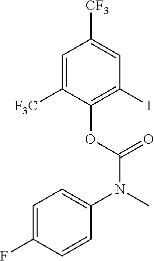 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80 °C for 4 hours. After completion of the reaction was confirmed by TLC (R_{f} −0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off-white solid (0.66 g, 93%).

### Step 6: Synthesis of 2-(3-(2-hydroxyethyl)-2-oxoimidazolidin-1-yl)-4,6-bis(trifluoromethyl)phenyl (4-fluorophenyl)(methyl)carbamate

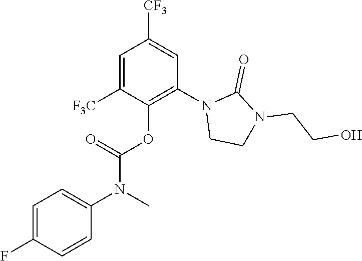 [SMILES: CN(C(=O)Oc1c(N2CCN(CCO)C2=O)cc(C(F)(F)F)cc1C(F)(F)F)c1ccc(F)cc1]

To a stirred solution of 2-iodo-4,6-bis(trifluoromethyl)phenyl (4-fluorophenyl)(methyl)carbamate (0.2 g, 0.0004 mol), 1-(2-hydroxyethyl)imidazolidin-2-one (0.1 g, 0.00079 mol), potassium carbonate (0.11 g, 0.00079 mol) and caesium fluoride (0.12 g, 0.00079 mol), were suspended in 1,4-dioxane (16 mL) priorly purged with N2 for 30 minutes, then copper (I)-iodide (0.038 g, 0.0002 mol), N,N′-dimethylethylenediamine (0.035 g, 0.42 mL, 0.0004 mol) were added. The reaction mixture was heated at 100 °C in an oil-bath for 24 h. After completion of the reaction by TLC (R_{f} 0.4, 5% MeOH in DCM) the reaction mixture was concentrated under reduced pressure. The residue obtained was purified by column chromatography (100-200 silica gel, 1-2% MeOH in DCM as eluent) to afford white sticky mass (40 mg, 20%).

### Step 7: Synthesis of 2-(3-(2-(((4-fluorophenyl)(methyl)carbamoyl)oxy)-3,5-bis(trifluoromethyl)phenyl)-2-oxoimidazolidin-1-yl)ethyl sulfamate

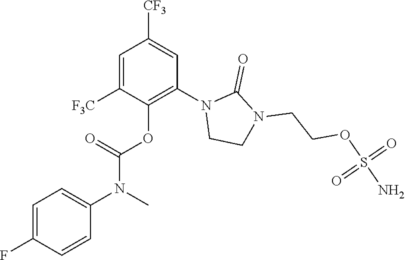 [SMILES: CN(C(=O)Oc1c(N2CCN(CCOS(N)(=O)=O)C2=O)cc(C(F)(F)F)cc1C(F)(F)F)c1ccc(F)cc1]

To a stirred solution of 2-(3-(2-hydroxyethyl)-2-oxoimidazolidin-1-yl)-4,6-bis(trifluoromethyl)phenyl (4-fluorophenyl)(methyl)carbamate (0.15 g, 0.00029 mol), triethylamine (0.28 ml, 0.00206 mol) were suspended in THF (6 mL). Reaction mass was cooled to −10 °C then sulfamoyl chloride (0.17 g, 0.00147 mol) was added and stirred for 15 min at this temperature and then continued stirring at RT for 4 h.

After completion of the reaction was confirmed by TLC (R_{f} −0.6, 100% EtOAc). Reaction mixture was concentrated under reduced pressure to afford the crude. The crude obtained was purified by column chromatography (100-200 silica gel and 85-90% EtOAc in Hexane as eluent) to afford the title compound as off-white solid (0.055 g, 32%).

¹H NMR (400 MHz, Trifluoroacetic acid-D) δ 7.92 (m, 2H), 7.35 (dd, J=8.7 Hz, J=4.6 Hz, 1H), 7.26 (q, J=4.4 Hz, 1H), 7.08 (m, 2H), 4.51 (t, J=4.3 Hz, 2H), 3.89 (m, 6H), 3.46 (d, J=60.3 Hz, 3H); MS(ESI): m/z 611.8 (M+H+22)⁺.

### Example 22

### 2,4-bis(trifluoromethyl)-6-(3-((3-hydroxyazetidin-3-yl)methyl)-2-oxoimidazolidin-1-yl)phenyl 4-fluorophenylmethylcarbamate

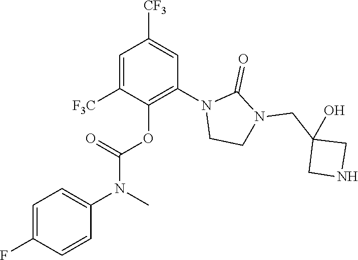 [SMILES: CN(C(=O)Oc1c(N2CCN(CC3(O)CNC3)C2=O)cc(C(F)(F)F)cc1C(F)(F)F)c1ccc(F)cc1]

### Step 1: Synthesis of 2-(2,4-bis(trifluoromethyl)phenyl)-4,4,5,5-tetramethyl-1,3,2-dioxaborolane

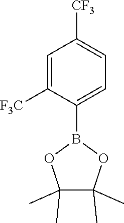 [SMILES: CC1(C)OB(c2ccc(C(F)(F)F)cc2C(F)(F)F)OC1(C)C]

A solution of 1-bromo-2,4-bis(trifluoromethyl)benzene (3 g, 0.01 mol) in 1,4 dioxane (90 mL) was purged with N2 in a sealed tube for 30 min and then added bispinacolato diboron (5.07 g, 0.02 mol), potassium acetate (1.96 g, 0.02 mol) and Pd(dppf)Cl₂·DCM (0.816 g, 0.001 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4×75 mL); washings collected was concentrated under reduced pressure to afford the yellow sticky solid (crude 6 g, Quantitative). The product was taken as such to next step without further purification.

### Step 2: Synthesis of 2,4-bis(trifluoromethyl)phenol

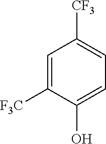 [SMILES: Oc1ccc(C(F)(F)F)cc1C(F)(F)F]

To a cold (0 °C) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×100 mL). The organic layer collected dried over anhydrous Na₂SO₄, concentrated under reduced pressure to afford the crude. The crude was purified by column chromatography (100-200 silica gel, 10-15% EtOAc in Hexane as eluent) to afford the title compound as pale yellow liquid (1.5 g, 65% yield).

### Step 3: Synthesis of 2-iodo-4,6-bis(trifluoromethyl)phenol

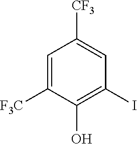 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20:6 mL) was cooled 0 °C in an ice bath with stirring. After 15 minutes, was added 12 (1.43 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 30% EtOAc in Hexane followed by 30% DCM in Hexane), the reaction mixture was cooled to 0 °C and then quenched with aq. sodium metabisulfite solution followed by extraction with EtOAc (2×100 mL). The organic layer collected dried over anhydrous Na₂SO₄, filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 4: Synthesis of (4-fluorophenyl)(methyl)carbamic chloride

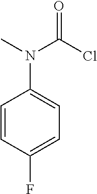

To a cold (0 °C) solution of triphosgene (1.6 g, 0.0128 mol) in DCM (40 mL) added a solution of N-methyl-4-fluoro aniline (1.89 g, 0.0064 mol) and pyridine (2.52 g, 2.6 mL, 0.032 mol) dropwise for over a period of 10 min. After that continued stirring at RT for 16 h. Progress of the reaction was monitored by TLC (R_{f} −0.7, 10% EtOAc in Hexane (×4)). After completion of the reaction, quenched the reaction mixture with 1M aq·HCl (50 mL) and then extracted with DCM (2×50 mL). The DCM layer separated was dried over anhydrous Na₂SO₄, filtered, and concentrated under reduced pressure to afford the title compound as green solid (1.8 g, 75%).

### Step 5: Synthesis of 2-iodo-4,6-bis(trifluoromethyl)phenyl (4-fluorophenyl)(methyl)carbamate

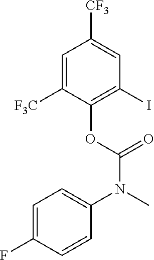 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80 °C for 4 hours. After completion of the reaction was confirmed by TLC (R_{f} −0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off-white solid (0.66 g, 93%).

### Step 6: Synthesis of 2-(2-oxoimidazolidin-1-yl)-4,6-bis(trifluoromethyl)phenyl (4-fluorophenyl)(methyl) carbamate

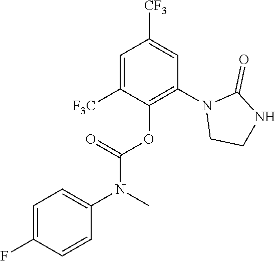 [SMILES: CN(C(=O)Oc1c(N2CCNC2=O)cc(C(F)(F)F)cc1C(F)(F)F)c1ccc(F)cc1]

2-iodo-4,6-bis(trifluoromethyl)phenyl (4-fluorophenyl)(methyl)carbamate (0.2 g, 0.00039 mol), 2-imidazilidinone (0.064 g, 0.0007 mol), copper (I)-iodide (0.037 g, 0.00019 mol), N,N′-dimethylethylenediamine (0.07 mL, 0.00039 mol), caesium fluoride (0.12 g, 0.00079 mol) and potassium carbonate (0.1 g, 0.00072 mol) were suspended in 1,4-dioxane priorly purged with N2 for 30 minutes (16 mL). The reaction mixture was heated at 90 °C in an oil-bath for 24 h. After completion of the reaction by TLC (R_{f} 0.4, 5% MeOH in DCM) the reaction mixture was concentrated under reduced pressure. The residue obtained was purified by column chromatography (100-200 silica gel, 0-3% MeOH in DCM as eluent) to afford the title compound as off-white solid (50 mg). The solid was re-purified by preparative TLC (solid phase: merck, 20×20 cm, silicagel 60 GF₂₅₄, 1 mm, PLC glass plate, 2% MeOH in DCM as eluent) to afford white solid (28 mg, 15%).

### Step 7: Synthesis of tert-butyl 3-((3-(2-(((4-fluorophenyl)(methyl)carbamoyl)oxy)-3,5-bis(trifluoromethyl)phenyl)-2-oxoimidazolidin-1-yl)methyl)-3-hydroxyazetidine-1-carboxylate

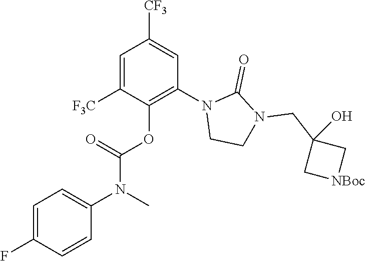 [SMILES: CN(C(=O)Oc1c(N2CCN(CC3(O)CN(C(=O)OC(C)(C)C)C3)C2=O)cc(C(F)(F)F)cc1C(F)(F)F)c1ccc(F)cc1]

2-(2-oxoimidazolidin-1-yl)-4,6-bis(trifluoromethyl)phenyl (4-fluorophenyl) (methyl) carbamate (0.1 g, 0.0002 mol) was dissolved in DMF (5 mL) was purged with N2 gas with stirring for 10 min. To the above solution added caesium carbonate (0.14 g, 0.00042 mol) and tert-butyl 1-oxa-5-azaspiro[2.3]hexane-5-carboxylate (0.079 g, 0.00042 mol) and again continued purging for 5 min at RT. After 24 h., completion of the reaction was confirmed by TLC (R_{f} −0.6, 60% EA/Hexane). Reaction mixture was quenched with cold water (50 mL) followed by extraction with ethyl acetate (2*50 mL). The organic layer separated was combined, dried over anhydrous Na₂SO₄, filtered, and concentrated under reduced pressure to afford the crude. The crude obtained was purified by preparative TLC (solid phase: merck, 20×20 cm, silicagel 60 GF₂₅₄, 1 mm, PLC glass plate, 60% EtOAc in Hexane as eluent) to afford sticky white solid. That sticky white solid was washed with pentane for 3-4 times to get the title compound as white solid (40 mg, 30%).

### Step 8: Synthesis of 2-(3-((3-hydroxyazetidin-3-yl)methyl)-2-oxoimidazolidin-1-yl)-4,6-bis(trifluoromethyl)phenyl (4-fluorophenyl)(methyl)carbamate

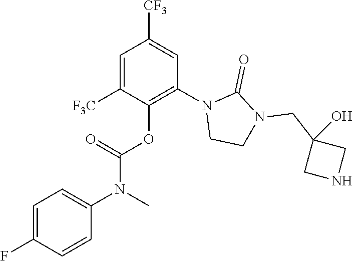 [SMILES: CN(C(=O)Oc1c(N2CCN(CC3(O)CNC3)C2=O)cc(C(F)(F)F)cc1C(F)(F)F)c1ccc(F)cc1]

A stirred solution of tert-butyl 3-((3-(2-(((4-fluorophenyl)(methyl)carbamoyl)oxy)-3,5-bis(trifluoromethyl)phenyl)-2-oxoimidazolidin-1-yl)methyl)-3-hydroxyazetidine-1-carboxylate (0.05 g, 0.000076 mol) in DCM was cooled to 0 °C. To the above cold solution added TFA (0.035 mL, 0.05 g, 0.00043 mol) and continued stirring at RT for 5 h. Completion of the reaction was confirmed by TLC (R_{f} −0.3, 10% MeOH/DCM). The reaction mixture was directly concentrated under reduced pressure to afford crude. The crude obtained was purified by preparative TLC (solid phase: merck, 20×20 cm, silicagel 60 GF₂₅₄, 1 mm, PLC glass plate, 10% MeOH in DCM as eluent) to afford sticky white solid, which was further washed with pentane for 2 to 3 times to get the title compound as white solid (TFA salt 25 mg, 59%).

¹H NMR (400 MHz, Trifluoroacetic acid-D) δ 8.01 (d, J=26.8 Hz, 1H), 7.89 (d, J=18.3 Hz, 1H), 7.36 (d, J=7.4 Hz, 1H), 7.27 (s, 1H), 7.13 (m, 2H), 4.55 (m, 4H), 4.00 (m, 6H), 3.50 (d, J=65.3 Hz, 3H); MS(ESI): m/z 551.35 (M+H)⁺.

### Example 23

### tert-butyl 3-((3-(2-(((4-fluorophenyl)(methyl)carbamoyl)oxy)-3,5-bis(trifluoromethyl)phenyl)-2-oxoimidazolidin-1-yl)methyl)-3-hydroxyazetidine-1-carboxylate

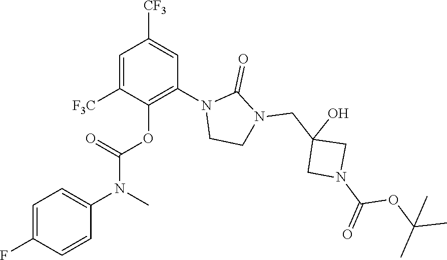 [SMILES: CN(C(=O)Oc1c(N2CCN(CC3(O)CN(C(=O)OC(C)(C)C)C3)C2=O)cc(C(F)(F)F)cc1C(F)(F)F)c1ccc(F)cc1]

### Step 1: Synthesis of 2-(2,4-bis(trifluoromethyl)phenyl)-4,4,5,5-tetramethyl-1,3,2-dioxaborolane

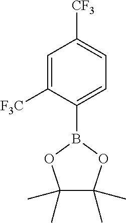 [SMILES: CC1(C)OB(c2ccc(C(F)(F)F)cc2C(F)(F)F)OC1(C)C]

A solution of 1-bromo-2,4-bis(trifluoromethyl)benzene (3 g, 0.01 mol) in 1,4 dioxane (90 mL) was purged with N2 in a sealed tube for 30 min and then added bispinacolato diboron (5.07 g, 0.02 mol), potassium acetate (1.96 g, 0.02 mol) and Pd(dppf)Cl₂·DCM (0.816 g, 0.001 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4-75 mL); washings collected was concentrated under reduced pressure to afford the yellow sticky solid (crude 6 g, Quantitative). The product was taken as such to next step without further purification.

### Step 2: Synthesis of 2,4-bis(trifluoromethyl)phenol

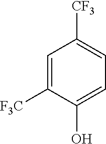 [SMILES: Oc1ccc(C(F)(F)F)cc1C(F)(F)F]

To a cold (0 °C) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×100 mL). The organic layer collected dried over anhydrous Na₂SO₄, concentrated under reduced pressure to afford the crude. The crude was purified by column chromatography (100-200 silica gel, 10-15% EtOAc in Hexane as eluent) to afford the title compound as pale yellow liquid (1.5 g, 65% yield).

### Step 3: Synthesis of 2-iodo-4,6-bis(trifluoromethyl)phenol

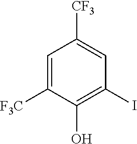 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20:6 mL) was cooled 0 °C in an ice bath with stirring. After 15 minutes, was added 12 (1.43 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 30% EtOAc in Hexane followed by 30% DCM in Hexane), the reaction mixture was cooled to 0 °C and then quenched with aq. sodium metabisulfite solution followed by extraction with EtOAc (2×100 mL). The organic layer collected dried over anhydrous Na₂SO₄, filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 4: Synthesis of (4-fluorophenyl)(methyl)carbamic chloride

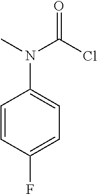

To a cold (0 °C) solution of triphosgene (1.6 g, 0.0128 mol) in DCM (40 mL) added a solution of N-methyl-4-fluoro aniline (1.89 g, 0.0064 mol) and pyridine (2.52 g, 2.6 mL, 0.032 mol) dropwise for over a period of 10 min. After that continued stirring at RT for 16 h. Progress of the reaction was monitored by TLC (R_{f} −0.7, 100/a EtOAc in Hexane (×4)). After completion of the reaction, quenched the reaction mixture with 1M aq·HCl (50 mL) and then extracted with DCM (2×50 mL). The DCM layer separated was dried over anhydrous Na₂SO₄, filtered, and concentrated under reduced pressure to afford the title compound as green solid (1.8 g, 75%).

### Step 5: Synthesis of 2-iodo-4,6-bis(trifluoromethyl)phenyl (4-fluorophenyl)(methyl)carbamate

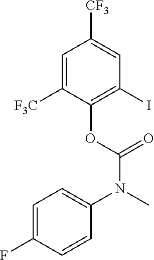 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80 °C for 4 hours. After completion of the reaction was confirmed by TLC (R_{f} −0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off-white solid (0.66 g, 93%).

### Step 6: Synthesis of 2-(2-oxoimidazolidin-1-yl)-4,6-bis(trifluoromethyl)phenyl (4-fluorophenyl)(methyl) carbamate

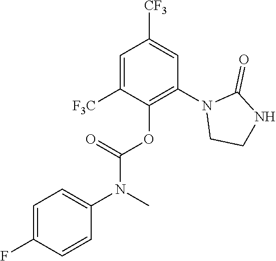 [SMILES: CN(C(=O)Oc1c(N2CCNC2=O)cc(C(F)(F)F)cc1C(F)(F)F)c1ccc(F)cc1]

2-iodo-4,6-bis(trifluoromethyl)phenyl (4-fluorophenyl)(methyl)carbamate (0.2 g, 0.00039 mol), 2-imidazilidinone (0.064 g, 0.0007 mol), copper (I)-iodide (0.037 g, 0.00019 mol), N,N′-dimethylethylenediamine (0.07 mL, 0.00039 mol), caesium fluoride (0.12 g, 0.00079 mol) and potassium carbonate (0.1 g, 0.00072 mol) were suspended in 1,4-dioxane priorly purged with N2 for 30 minutes (16 mL). The reaction mixture was heated at 90 °C in an oil-bath for 24 h. After completion of the reaction by TLC (R_{f} 0.4, 5% MeOH in DCM) the reaction mixture was concentrated under reduced pressure. The residue obtained was purified by column chromatography (100-200 silica gel, 0-3% MeOH in DCM as eluent) to afford the title compound as off-white solid (50 mg). The solid was re-purified by preparative TLC (solid phase: merck, 20×20 cm, silicagel 60 GF₂₅₄, 1 mm, PLC glass plate, 2% MeOH in DCM as eluent) to afford white solid (28 mg, 15%).

### Step 7: Synthesis of tert-butyl 3-((3-(2-(((4-fluorophenyl)(methyl)carbamoyl)oxy)-3,5-bis(trifluoromethyl)phenyl)-2-oxoimidazolidin-1-yl)methyl)-3-hydroxyazetidine-1-carboxylate

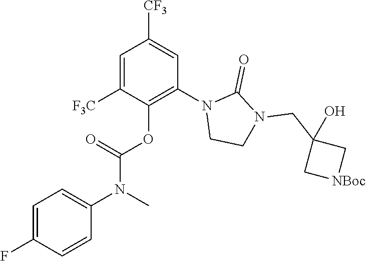 [SMILES: CN(C(=O)Oc1c(N2CCN(CC3(O)CN(C(=O)OC(C)(C)C)C3)C2=O)cc(C(F)(F)F)cc1C(F)(F)F)c1ccc(F)cc1]

2-(2-oxoimidazolidin-1-yl)-4,6-bis(trifluoromethyl)phenyl (4-fluorophenyl) (methyl) carbamate (0.1 g, 0.0002 mol) was dissolved in DMF (5 mL) was purged with N2 gas with stirring for 10 min. To the above solution added caesium carbonate (0.14 g, 0.00042 mol) and tert-butyl 1-oxa-5-azaspiro[2.3]hexane-5-carboxylate (0.079 g, 0.00042 mol) and again continued purging for 5 min at RT. After 24 h., completion of the reaction was confirmed by TLC (R_{f} −0.6, 60% EA/Hexane). Reaction mixture was quenched with cold water (50 mL) followed by extraction with ethyl acetate (2*50 mL). The organic layer separated was combined, dried over anhydrous Na₂SO₄, filtered, and concentrated under reduced pressure to afford the crude. The crude obtained was purified by preparative TLC (solid phase: merck, 20×20 cm, silicagel 60 GF₂₅₄, 1 mm, PLC glass plate, 60% EtOAc in Hexane as eluent) to afford sticky white solid. That sticky white solid was washed with pentane for 3-4 times to get the title compound as white solid (40 mg, 30%).

¹H NMR (400 MHz, Acetone-d6) δ 8.22 (d, J=1.2 Hz, 1H), 7.90 (m, 1H), 7.54 (s, 2H), 7.22 (s, 2H), 3.93 (s, 4H), 3.76 (m, 4H), 3.58 (m, 2H), 3.36 (s, 3H), 1.39 (s, 9H); m/z 649.1 (M−H)⁺.

### Example 24

### 2-(3-(3-(dimethylamino)-2-hydroxypropyl)-2-oxoimidazolidin-1-yl)-4,6-bis(trifluoromethyl)phenyl (4-fluorophenyl)(methyl)carbamate

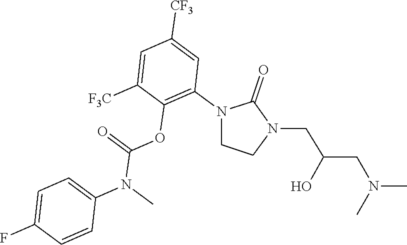 [SMILES: CN(C)CC(O)CN1CCN(c2cc(C(F)(F)F)cc(C(F)(F)F)c2OC(=O)N(C)c2ccc(F)cc2)C1=O]

### Step 1: Synthesis of 2-(2,4-bis(trifluoromethyl)phenyl)-4,4,5,5-tetramethyl-1,3,2-dioxaborolane

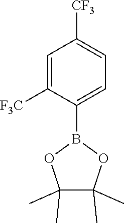 [SMILES: CC1(C)OB(c2ccc(C(F)(F)F)cc2C(F)(F)F)OC1(C)C]

A solution of 1-bromo-2,4-bis(trifluoromethyl)benzene (3 g, 0.01 mol) in 1,4 dioxane (90 mL) was purged with N2 in a sealed tube for 30 min and then added bispinacolato diboron (5.07 g, 0.02 mol), potassium acetate (1.96 g, 0.02 mol) and Pd(dppf)Cl₂·DCM (0.816 g, 0.001 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4×75 mL); washings collected was concentrated under reduced pressure to afford the yellow sticky solid (crude 6 g, Quantitative). The product was taken as such to next step without further purification.

### Step 2: Synthesis of 2,4-bis(trifluoromethyl)phenol

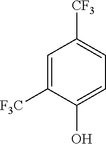 [SMILES: Oc1ccc(C(F)(F)F)cc1C(F)(F)F]

To a cold (0 °C) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×100 mL). The organic layer collected dried over anhydrous Na₂SO₄, concentrated under reduced pressure to afford the crude. The crude was purified by column chromatography (100-200 silica gel, 10-15% EtOAc in Hexane as eluent) to afford the title compound as pale yellow liquid (1.5 g, 65% yield).

### Step 3: Synthesis of 2-iodo-4,6-bis(trifluoromethyl)phenol

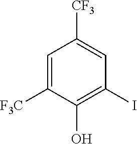 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20:6 mL) was cooled 0 °C in an ice bath with stirring. After 15 minutes, was added 12 (1.43 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 30% EtOAc in Hexane followed by 30% DCM in Hexane), the reaction mixture was cooled to 0 °C and then quenched with aq. sodium metabisulfite solution followed by extraction with EtOAc (2×100 mL). The organic layer collected dried over anhydrous Na₂SO₄, filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 4: Synthesis of (4-fluorophenyl)(methyl)carbamic chloride

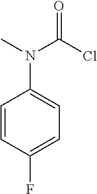

To a cold (0 °C) solution of triphosgene (1.6 g, 0.0128 mol) in DCM (40 mL) added a solution of N-methyl-4-fluoro aniline (1.89 g, 0.0064 mol) and pyridine (2.52 g, 2.6 mL, 0.032 mol) dropwise for over a period of 10 min. After that continued stirring at RT for 16 h. Progress of the reaction was monitored by TLC (R_{f} −0.7, 10% EtOAc in Hexane (×4)). After completion of the reaction, quenched the reaction mixture with 1M aq·HCl (50 mL) and then extracted with DCM (2×50 mL). The DCM layer separated was dried over anhydrous Na₂SO₄, filtered, and concentrated under reduced pressure to afford the title compound as green solid (1.8 g, 75%).

### Step 5: Synthesis of 2-iodo-4,6-bis(trifluoromethyl)phenyl (4-fluorophenyl)(methyl)carbamate

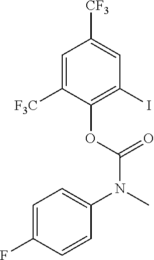 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80 °C for 4 hours. After completion of the reaction was confirmed by TLC (R_{f} −0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off-white solid (0.66 g, 93%).

### Step 6: Synthesis of 2-(2-oxoimidazolidin-1-yl)-4,6-bis(trifluoromethyl)phenyl (4-fluorophenyl)methyl) carbamate

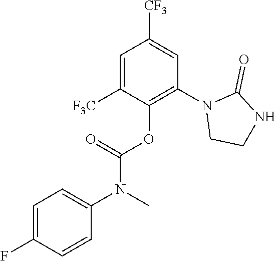 [SMILES: CN(C(=O)Oc1c(N2CCNC2=O)cc(C(F)(F)F)cc1C(F)(F)F)c1ccc(F)cc1]

2-iodo-4,6-bis(trifluoromethyl)phenyl (4-fluorophenyl)(methyl)carbamate (0.2 g, 0.00039 mol), 2-imidazilidinone (0.064 g, 0.0007 mol), copper (I)-iodide (0.037 g, 0.00019 mol), N,N′-dimethylethylenediamine (0.07 mL, 0.00039 mol), caesium fluoride (0.12 g, 0.00079 mol) and potassium carbonate (0.1 g, 0.00072 mol) were suspended in 1,4-dioxane priorly purged with N2 for 30 minutes (16 mL). The reaction mixture was heated at 90 °C in an oil-bath for 24 h. After completion of the reaction by TLC (R_{f} 0.4, 5% MeOH in DCM) the reaction mixture was concentrated under reduced pressure. The residue obtained was purified by column chromatography (100-200 silica gel, 0-3% MeOH in DCM as eluent) to afford the title compound as off-white solid (50 mg). The solid was re-purified by preparative TLC (solid phase: merck, 20×20 cm, silicagel 60 GF₂₅₄, 1 mm, PLC glass plate, 2% MeOH in DCM as eluent) to afford white solid (28 mg, 15%).

### Step 7: Synthesis of 2-(3-(oxiran-2-ylmethyl)-2-oxoimidazolidin-1-yl)-4,6-bis(trifluoromethyl)phenyl (4-fluorophenyl)(methyl)carbamate

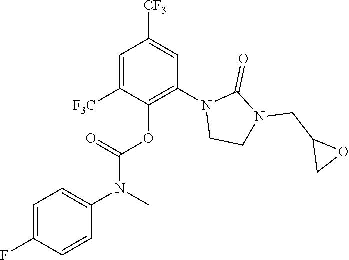 [SMILES: CN(C(=O)Oc1c(N2CCN(CC3CO3)C2=O)cc(C(F)(F)F)cc1C(F)(F)F)c1ccc(F)cc1]

To a stirred solution of 2-(2-oxoimidazolidin-1-yl)-4,6-bis(trifluoromethyl)phenyl (4-fluorophenyl)(methyl)carbamate (0.1 g, 0.0002 mol) in DMF (8 mL) was purged with N2 for 10 minutes. To the above solution added K₃PO₄ (0.27 g, 0.00128 mol) at RT and continued purging for 10 min. After 30 min of stirring at RT, reaction mixture was cooled to 0 °C and to the same added epibromohydrin (0.12 mL, 0.00128 mol) and KI (0.13 g, 0.0008 mol). The reaction mixture was stirred at RT for 20 h. After completion of the reaction confirmed by TLC (R_{f} 0.3, 60% EtOAc in Hexane) the reaction mixture was diluted with cold water (150 mL) followed by extraction with ethyl acetate (2*100 mL). The organic fractions separated were combined, dried over anhydrous Na₂SO₄, filtered and concentrated under reduced pressure to afford crude. The crude residue was purified by preparative TLC (solid phase: merck, 20×20 cm, silicagel 60 GF₂₅₄, 1 mm, PLC glass plate, 50% EtOAc in Hexane as eluent) to afford the desired title compound as sticky oil (40 mg, 38%).

### Step 8: Synthesis of 2-(3-(3-(dimethylamino)-2-hydroxypropyl)-2-oxoimidazolidin-1-yl)-4,6-bis (trifluoromethyl)phenyl(4-fluorophenyl)(methyl)carbamate

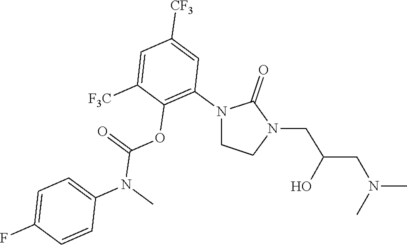 [SMILES: CN(C)CC(O)CN1CCN(c2cc(C(F)(F)F)cc(C(F)(F)F)c2OC(=O)N(C)c2ccc(F)cc2)C1=O]

To a stirred solution of 2-(3-(oxiran-2-ylmethyl)-2-oxoimidazolidin-1-yl)-4,6-bis(trifluoromethyl) phenyl (4-fluorophenyl)(methyl)carbamate (0.1 g, 0.00019 mol) in methanol (5 mL) under N2 atmosphere was added a solution of dimethylamine (2 M in THF, 4.75 mL, 0.0095 mol) at 0 °C and after addition continued stirring at RT for 6 h. After completion of the reaction confirmed by TLC (R_{f} 0.2, 7% MeOH in DCM), the reaction mixture was directly concentrated under reduced pressure at 40 °C to afford crude. The impure residue was purified by preparative TLC (solid phase: merck, 20×20 cm, silicagel 60 GF₂₅₄, 1 mm, PLC glass plate, 5% MeOH in DCM as eluent) to afford the desired title compound as sticky off-white solid (40 mg, 37%).

¹H NMR (400 MHz, DMSO-d₆) δ 8.2 (m, 1H), 7.93 (d, J=21.6 Hz, 1H), 7.44 (s, 2H), 7.28 (t, J=8.5 Hz, 2H), 4.7 (d, J=9.9 Hz, 1H), 3.82 (d, J=22.1 Hz, 2H), 3.46 (m, 3H), 3.42 (s, 2H), 3.07 (s, 1H), 2.28 (s, 2H), 2.20 (s, 6H), 1,29 (m, 1H); MS(ESI): m/z 567.35 (M+H)⁺.

### Example 25

### 2,4-bis(trifluoromethyl)-6-(3-((oxiran-2-yl)methyl)-2-oxoimidazolidin-1-yl)phenyl (4-fluorophenyl)(methyl)carbamate

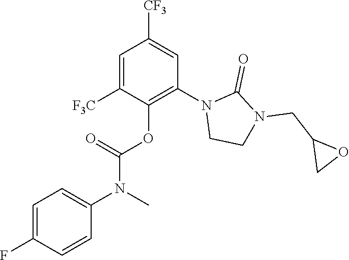 [SMILES: CN(C(=O)Oc1c(N2CCN(CC3CO3)C2=O)cc(C(F)(F)F)cc1C(F)(F)F)c1ccc(F)cc1]

### Step 1: Synthesis of 2-(2,4-bis(trifluoromethyl)phenyl)-4,4,5,5-tetramethyl-1,3,2-dioxaborolane

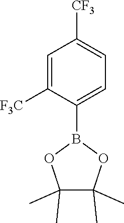 [SMILES: CC1(C)OB(c2ccc(C(F)(F)F)cc2C(F)(F)F)OC1(C)C]

A solution of 1-bromo-2,4-bis(trifluoromethyl)benzene (3 g, 0.01 mol) in 1,4 dioxane (90 mL) was purged with N2 in a sealed tube for 30 min and then added bispinacolato diboron (5.07 g, 0.02 mol), potassium acetate (1.96 g, 0.02 mol) and Pd(dppf)Cl₂·DCM (0.816 g, 0.001 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4×75 mL); washings collected was concentrated under reduced pressure to afford the yellow sticky solid (crude 6 g, Quantitative). The product was taken as such to next step without further purification.

### Step 2: Synthesis of 2,4-bis(trifluoromethyl)phenol

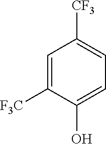 [SMILES: Oc1ccc(C(F)(F)F)cc1C(F)(F)F]

To a cold (0 °C) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×100 mL). The organic layer collected dried over anhydrous Na₂SO₄, concentrated under reduced pressure to afford the crude. The crude was purified by column chromatography (100-200 silica gel, 10-15% EtOAc in Hexane as eluent) to afford the title compound as pale yellow liquid (1.5 g, 65% yield).

### Step 3: Synthesis of 2-iodo-4,6-bis(trifluoromethyl)phenol

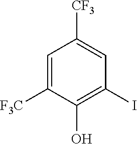 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20:6 mL) was cooled 0 °C in an ice bath with stirring. After 15 minutes, was added 12 (1.43 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 30% EtOAc in Hexane followed by 30% DCM in Hexane), the reaction mixture was cooled to 0 °C and then quenched with aq. sodium metabisulfite solution followed by extraction with EtOAc (2×100 mL). The organic layer collected dried over anhydrous Na₂SO₄, filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 4: Synthesis of (4-fluorophenyl)(methyl)carbamic chloride

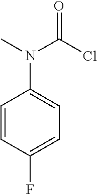

To a cold (0 °C) solution of triphosgene (1.6 g, 0.0128 mol) in DCM (40 mL) added a solution of N-methyl-4-fluoro aniline (1.89 g, 0.0064 mol) and pyridine (2.52 g, 2.6 mL, 0.032 mol) dropwise for over a period of 10 min. After that continued stirring at RT for 16 h. Progress of the reaction was monitored by TLC (R_{f} −0.7, 10% EtOAc in Hexane (×4)). After completion of the reaction, quenched the reaction mixture with 1M aq·HCl (50 mL) and then extracted with DCM (2×50 mL). The DCM layer separated was dried over anhydrous Na₂SO₄, filtered, and concentrated under reduced pressure to afford the title compound as green solid (1.8 g, 75%).

### Step 5: Synthesis of 2-iodo-4,6-bis(trifluoromethyl)phenyl (4-fluorophenyl)(methyl)carbamate

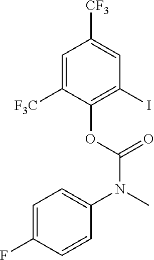 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80 °C for 4 hours. After completion of the reaction was confirmed by TLC (R_{f} −0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off-white solid (0.66 g, 93%).

### Step 6: Synthesis of 2-(2-oxoimidazolidin-1-yl)-4,6-bis(trifluoromethyl)phenyl (4-fluorophenyl)methyl) carbamate

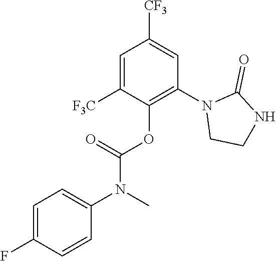 [SMILES: CN(C(=O)Oc1c(N2CCNC2=O)cc(C(F)(F)F)cc1C(F)(F)F)c1ccc(F)cc1]

2-iodo-4,6-bis(trifluoromethyl)phenyl (4-fluorophenyl)(methyl)carbamate (0.2 g, 0.00039 mol), 2-imidazilidinone (0.064 g, 0.0007 mol), copper (I)-iodide (0.037 g, 0.00019 mol), N,N′-dimethylethylenediamine (0.07 mL, 0.00039 mol), caesium fluoride (0.12 g, 0.00079 mol) and potassium carbonate (0.1 g, 0.00072 mol) were suspended in 1,4-dioxane priorly purged with N2 for 30 minutes (16 mL). The reaction mixture was heated at 90 °C in an oil-bath for 24 h. After completion of the reaction by TLC (R_{f} 0.4, 5% MeOH in DCM) the reaction mixture was concentrated under reduced pressure. The residue obtained was purified by column chromatography (100-200 silica gel, 0-3% MeOH in DCM as eluent) to afford the title compound as off-white solid (50 mg). The solid was re-purified by preparative TLC (solid phase: merck, 20×20 cm, silicagel 60 GF₂₅₄, 1 mm, PLC glass plate, 2% MeOH in DCM as eluent) to afford white solid (28 mg, 15%).

### Step 7: Synthesis of 2-(3-(oxiran-2-ylmethyl)-2-oxoimidazolidin-1-yl)-4,6-bis(trifluoromethyl)phenyl (4-fluorophenyl)(methyl)carbamate

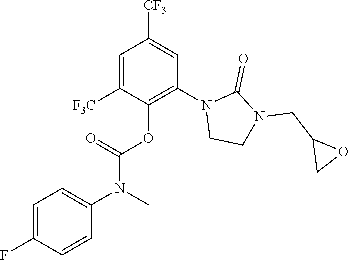 [SMILES: CN(C(=O)Oc1c(N2CCN(CC3CO3)C2=O)cc(C(F)(F)F)cc1C(F)(F)F)c1ccc(F)cc1]

To a stirred solution of 2-(2-oxoimidazolidin-1-yl)-4,6-bis(trifluoromethyl)phenyl (4-fluorophenyl)(methyl)carbamate (0.1 g, 0.0002 mol) in DMF (8 mL) was purged with N2 for 10 minutes. To the above solution added K₃PO₄ (0.27 g, 0.00128 mol) at RT and continued purging for 10 min. After 30 min of stirring at RT, reaction mixture was cooled to 0 °C and to the same added epibromohydrin (0.12 mL, 0.00128 mol) and KI (0.13 g, 0.0008 mol). The reaction mixture was stirred at RT for 20 h. After completion of the reaction confirmed by TLC (R_{f} 0.3, 60% EtOAc in Hexane) the reaction mixture was diluted with cold water (150 mL) followed by extraction with ethyl acetate (2*100 mL). The organic fractions separated were combined, dried over anhydrous Na₂SO₄, filtered and concentrated under reduced pressure to afford crude. The crude residue was purified by preparative TLC (solid phase: merck, 20×20 cm, silicagel 60 GF₂₅₄, 1 mm, PLC glass plate, 50% EtOAc in Hexane as eluent) to afford the desired title compound as sticky oil (40 mg, 38%).

¹H NMR (500 MHz, DMSO-d₆) δ 8.27 (d, J=19.3 Hz, 1H),), 7.96 (d, J=35.1 Hz, 1H), 7.44 (s, 2H), 7.29 (s, 2H), 3.98 (d, J=112.1 Hz, 1H), 3.58 (s, 4H), 3.21 (m, 4H): MS(ESI): m/z 522.35 (M+H)⁺.

### Example 26

### 2-(2-oxoimidazolidin-1-yl)-4,6-bis(trifluoromethyl)phenyl (4-(difluoromethoxy)phenyl)(methyl)carbamate

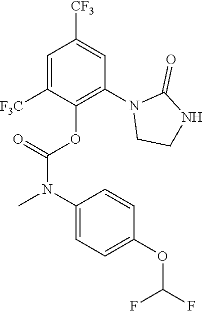 [SMILES: CN(C(=O)Oc1c(N2CCNC2=O)cc(C(F)(F)F)cc1C(F)(F)F)c1ccc(OC(F)F)cc1]

### Step 1: Synthesis of 4-(difluoromethoxy)-N-methylaniline

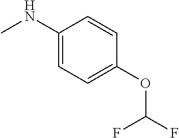

Sodium methoxide (5.82 mL of a 5.4 M solution in MeOH, 31.42 mmol) was added all at once to a solution of 4-(difluoromethoxy)aniline (779 uL, 6.28 mmol)) and paraformaldehyde (377 mg, 12.57 mmol) in anhydrous MeOH (24 mL) under nitrogen. This solution was stirred at 60 °C overnight and then cooled to room temperature. Sodium borohydride (594 mg, 15.71 mmol)) was added all at once and the reaction was stirred at room temperature overnight. The reaction was diluted with saturated aqueous ammonium chloride (100 mL) and extracted with DCM (3×). The combined organic extracts were dried over anhydrous sodium sulfate and concentrated to afford the titled compound as a dark red liquid (1.05 g, 96%).

¹H NMR (400 MHz, CDCl₃) δ 7.00 (m, 2H), 6.65 (m, 2H), 6.38 (t, J=74.7 Hz, 1H), 2.84 (s, 3H); MS(ESI): m/z 174.1 [(M+H)⁺].

### Step 2: Synthesis of (4-(difluoromethoxy)phenyl)(methyl)carbamic chloride

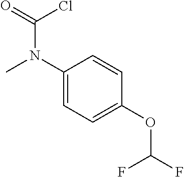

A solution of 4-(difluoromethoxy)-N-methylaniline (1.05 g, 6.06 mmol) and pyridine (0.98 mL, 12.13 mmol) in anhydrous DCM (20 mL) was cooled to 0 °C under nitrogen. A solution of triphosgene (900 mg, 3.03 mmol) in anhydrous DCM (5 mL) was added rapidly dropwise and the reaction was allowed to warm to RT with stirring over 90 minutes. The reaction solution was diluted with DCM, washed with 1N aqueous HCL twice, dried over anhydrous sodium sulfate and concentrated to afford the titled compound as a reddish-orange oil (1.42 g, 99%).

¹H NMR (400 MHz, CDCl₃) δ 7.26 (m, 2H), 7.18 (m, 2H), 6.54 (t, J=73.2 Hz, 1H), 3.32-3.57 (3H); MS(ESI): m/z 236.1 [(M+H)⁺].

### Step 3: Synthesis of 2-iodo-4,6-bis(trifluoromethyl)phenyl (4-(difluoromethoxy)phenyl)(methyl)carbamate

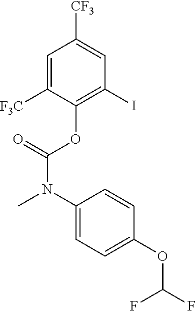 [SMILES: CN(C(=O)Oc1c(I)cc(C(F)(F)F)cc1C(F)(F)F)c1ccc(OC(F)F)cc1]

2-iodo-4,6-bis(trifluoromethyl)phenol (845 mg, 2.38 mmol) and (4-(difluoromethoxy)phenyl)(methyl)carbamic chloride (1400 mg, 5.94 mmol) were dissolved into anhydrous pyridine (20 mL). This solution was stirred at 90 °C for 4 hours, then cooled to room temperature and concentrated down. The residual solid was partitioned between EtOAc and 1N aqueous HCl. The aqueous phase was separated and extracted with EtOAc twice. The combined organic solutions were washed with brine, dried over anhydrous sodium sulfate and concentrated. The crude product was purified by column chromatography on silica gel using a gradient solvent system of 0 to 40% EtOAc in hexanes to afford the titled compound as a yellow oil (1.36 g, 100%).

¹H NMR (400 MHz, CDCl₃) δ 8.22-8.28 (1H), 7.85-7.92 (1H), 7.42 (m, 2H), 7.18 (m, 2H), 6.31-6.72 (m, 1H), 3.36-3.57 (3H); MS(ESI): m/z 556.0 [(M+H)⁺].

### Step 4: Synthesis of 2-(2-oxoimidazolidin-1-yl)-4,6-bis(trifluoromethyl)phenyl (4-(difluoromethoxy)phenyl)(methyl)carbamate

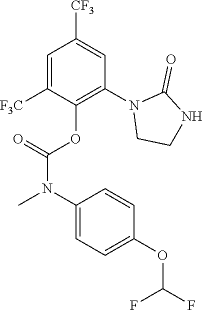 [SMILES: CN(C(=O)Oc1c(N2CCNC2=O)cc(C(F)(F)F)cc1C(F)(F)F)c1ccc(OC(F)F)cc1]

2-iodo-4,6-bis(trifluoromethyl)phenyl (4-(difluoromethoxy)phenyl)(methyl)carbamate (200 mg, 0.3603 mmol), 2-imidazolidinone (56 mg, 0.6466 mmol), copper (I) iodide (33 mg, 0.1756 mmol), cesium fluoride (111 mg, 0.7299 mmol), N,N′-dimethylethylenediamine (39 uL, 0.3603 mmol) and anhydrous potassium carbonate (92 mg, 0.6652 mmol) were added to degassed anhydrous 1,4-dioxane (4.0 mL) under nitrogen. The resulting suspension was stirred at 90 °C overnight. The reaction was cooled to room temperature, diluted with methanol, filtered and concentrated down to yield a gray solid. This was suspended in dichloromethane, filtered through plug of celite and concentrated down. The crude product was purified by column chromatography on silica gel using a gradient solvent system of 0 to 100% EtOAc in hexanes to afford the titled compound as a colorless oil (14 mg, 8%).

¹H NMR (400 MHz, CDCl₃) δ 7.75-7.85 (2H), 7.34 (m, 2H), 7.15 (m, 2H), 6.32-6.69 (bt, J=73.6 Hz, 1H), 3.60 (m, 2H), 3.49 (s, 3H), 3.37 (bs, 2H); ESIMS: m/z 514.4 [(M+H)⁺].

### Example 27

### 2-(3-methyl-2,4-dioxoimidazolidin-1-yl)-4,6-bis(trifluoromethyl)phenyl N-(4-fluorophenyl)-N-methylcarbamate

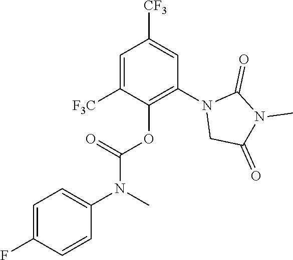 [SMILES: CN1C(=O)CN(c2cc(C(F)(F)F)cc(C(F)(F)F)c2OC(=O)N(C)c2ccc(F)cc2)C1=O]

### Step 1: Synthesis of 3-methylimidazolidine-2,4-dione

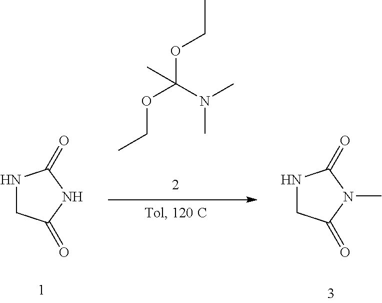

To a solution of compound 1 (2.0 g, 20.0 mmol, 1.0 eq) in toluene (120 mL) was added compound 2 (5.3 g, 40.0 mmol, 2.0 eq) at rt. The reaction mixture was heated at 120 °C for 2 h. The reaction was monitored by TLC and LC-MS. The reaction was cooled to rt, the crystals precipitated from toluene. The solid was filtered to give compound 3 (1.7 g, 85%).

### Step 2: Synthesis of 2-(2,4-bis(trifluoromethyl)phenyl)-4,4,5,5-tetramethyl-1,3,2-dioxaborolane

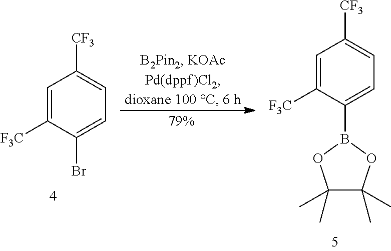 [SMILES: FC(F)(F)c1ccc(Br)c(C(F)(F)F)c1 ; "79%" ; "B2Pin2, KOAcPd(dppf)Cl2,dioxane 100 °C, 6 h" ; CC1(C)OB(c2ccc(C(F)(F)F)cc2C(F)(F)F)OC1(C)C]

A solution of 1-bromo-2,4-bis(trifluoromethyl)benzene (25.0 g, 85.3 mmol 1.0 eq) in dioxane (300 mL) was purged with N2 in a sealed tube for 2 min and then added bispinacolato diboron (43.4 g, 170 mmol, 2.0 eq), potassium acetate (16.8 g, 170 mmol) and Pd(dppf)Cl₂·DCM (2.4 g, 8.53 mmol, 0.1 eq) with stirring under inert atmosphere. The tightly closed sealed tube was heated to 100 °C in an oil bath for 16 h with stirring. Progress of the reaction was monitored by TLC (Rf=0.5, 10% EtOAc in hexane). After completion of the reaction, the reaction mixture was concentrated under reduced pressure. The residue was partitioned between ethyl acetate (200 mL) and water (150 mL). The organic layer was dried over sodium sulfate, and concentrated under reduced pressure. The residue was purified by silica gel chromatography eluted with PE:EA=1:20 to give compound 5 (23.0 g, 79%).

### Step 3: Synthesis of 2,4-bis(trifluoromethyl)phenol

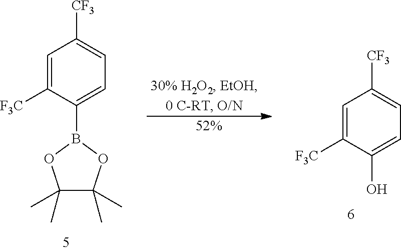 [SMILES: CC1(C)OB(c2ccc(C(F)(F)F)cc2C(F)(F)F)OC1(C)C ; "52%" ; "30% H2O2, EtOH,0 C-RT, O/N" ; Oc1ccc(C(F)(F)F)cc1C(F)(F)F]

To a solution of 2-(2,4-bis(trifluoromethyl)phenyl)-4,4,5,5-tetramethyl-1,3,2-dioxaborolane (23.0 g, 67.6 mmol, 1.0 eq) in ethanol (300 mL) at 0 °C was added hydrogen peroxide 30% aq. solution (24 mL) under inert atmosphere with stirring. The reaction mixture was stirred at ambient temperature for 16 h. After completion of the reaction confirmed by TLC (Rf 0.2, 10% EtOAc in hexane), the reaction mixture was cooled to 0 °C and quenched with aq. sodium metabisulfite solution followed by extraction with EtOAc (2×200 mL). The organic layer collected was washed with water followed by brine, dried over anhydrous Na₂SO₄, filtered and concentrated under reduced pressure to afford a crude. The crude obtained was purified by column chromatography (100-200 silica gel, 0-20% EtOAc in petroleum ether as eluent) to afford compound 6 (8.1, 52%) as pale-yellow liquid.

### Step 4: Synthesis of 2-iodo-4,6-bis(trifluoromethyl)phenol

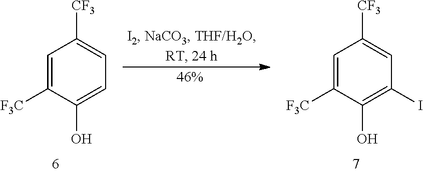 [SMILES: Oc1ccc(C(F)(F)F)cc1C(F)(F)F ; "46%" ; "I2, NaCO3, THF/H2O,RT, 24 h" ; Oc1c(I)cc(C(F)(F)F)cc1C(F)(F)F]

A solution of 2,4-bis(trifluoromethyl)phenol (8.0 g, 34.8 mol) in THF:H₂O (3:1, 160 mL) was cooled 0 °C in an ice bath with stirring. After 15 minutes, iodine (10.6 g, 41.7 mmol) was added, followed with Na₂CO₃ (4.4 g, 41.5 mmol) under inert atmosphere with stirring. The reaction mixture stirred at ambient temperature for 24 h. After completion of the reaction (TLC: R_{f} 0.35, 30% EtOAc in Hexane), the reaction mixture was cooled to 0 °C and then quenched with aq. sodium metabisulfite solution followed by extraction with EtOAc (2×150 mL). The organic layer collected was washed with water followed by brine, dried over anhydrous Na₂SO₄, filtered and concentrated under reduced pressure to afford a crude. The crude was purified by column chromatography (100-200 silica gel, 0-5% EtOAc in petroleum ether as eluent) to afford compound 7 (5.8 g, 46% yield) as pale-yellow solid.

### Step 5: Synthesis of 1-(2-hydroxy-3,5-bis(trifluoromethyl)phenyl)-3-methylimidazolidine-2,4-dione

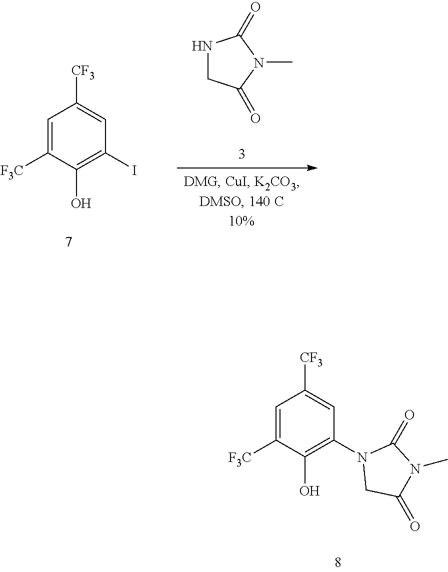

To a solution of compound 7 (1.0 g, 2.8 mmol, 1.0 eq) and compound 3 (480 mg, 4.2 mmol, 1.5 eq) in DMSO (10 mL) at rt was added CuI (106 mg, 0.56 mmol, 0.2 eq), NMG (160 mg, 1.12 mmol, 0.4 eq) and K₂CO₃ (800 mg, 5.6 mmol, 2.0 eq). The reaction mixture was heated at 140 °C in an oil-bath for 4 h. After completion of the reaction by TLC (R_{f} 0.3, PE:EA=1:1), water (20 mL) was added and adjust to pH 6 with HCl (1N). The mixture was extracted with EA (30 mL×2). The combined organic layer was washed with brine, dried over Na₂SO₄ and filtered. The filtration was concentrated under reduced pressure. The residue was purified by column chromatography (100-200 silica gel, 50% EA in PE as eluent) to afford compound 8 (100 mg, yield: 10%) as pale-yellow solid.

### Step 6: Synthesis of AB25859

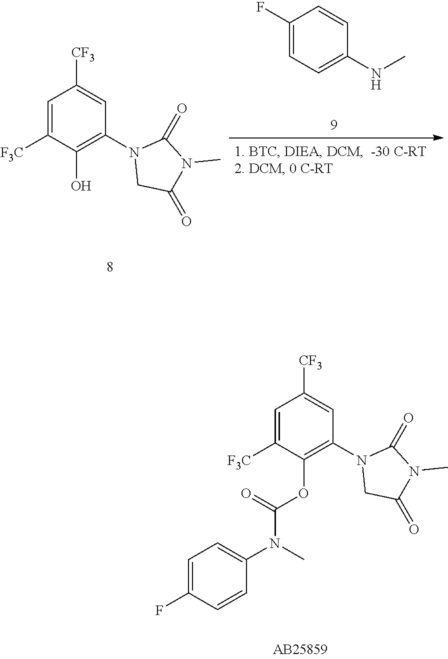

To a solution of compound 9 (100 mg, 0.32 mmol, 1.0 eq) in DCM (10 mL), was added DIEA (63 mg, 0.482 mmol, 1.5 eq), and triphosgene [125 mg, 0.418 mmol, 1.3 eq, in DCM (2 mL)] at −5 °C. The reaction mixture was stirred for 1 h at rt under N2. The reaction solution was concentrated under reduced pressure to remove the solvent. Then DCM (3 mL) added to above residue. The solution was used for next step.

### Step 7: Synthesis of 2-(3-methyl-2,4-dioxoimidazolidin-1-yl)-4,6-bis(trifluoromethyl)phenyl N-(4-fluorophenyl)-N-methylcarbamate

To a solution of 9 (243 mg, 0.48 mmol, 1.5 eq) in DCM (5 mL), was added the above residue solution at 0 °C. The mixture was stirred for 1 h at rt under N2. After completion of the reaction by TLC (R_{f} 0.5, PE:EA=2:1), HCl (1 N, 15 mL) was added. Then the mixture was extracted with DCM (20 mL×2). The combined organic layer was washed with brine, dried over Na₂SO₄ and filtered. The filtration was concentrated under reduced pressure. The residue was purified by Prep-HPLC to give title compound (AB25859; 36 mg, 27%) as off-white solid.

¹H NMR (400 MHz, CDCl₃): δ 7.89-7.82 (s, 2H), 7.11-7.08 (m, 2H), 7.24 (m, 2H), 4.33-4.24 (m, 2H), 3.47 (s, 1H) 3.30 (s, 2H), 3.14-3.13 (d, 3H).

### Example 28

### 2-(2-oxoimidazolidin-1-yl)-4,6-bis(trifluoromethyl)phenyl N-(4-fluoro-3-methylphenyl)-N-methylcarbamate

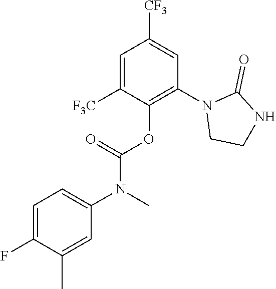 [SMILES: Cc1cc(N(C)C(=O)Oc2c(N3CCNC3=O)cc(C(F)(F)F)cc2C(F)(F)F)ccc1F]

### Step 1: Synthesis of 1-(2-hydroxy-3,5-bis(trifluoromethyl)phenyl)imidazolidine-2-one

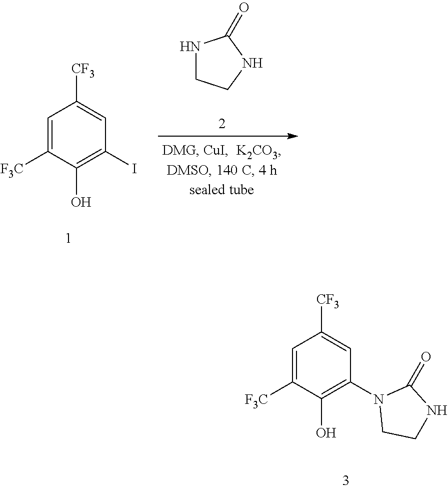

To a solution of compound 1 (1.2 g, 3.38 mmol, 1.0 eq) and compound 2 (581 mg, 6.76 mmol, 2.0 eq) in DMSO (10 mL) were added CuI (129 mg, 0.68 mmol, 0.2 eq), DMG (191 mg, 1.352 mmol, 0.4 eq) and K₂CO₃ (940 mg, 6.76 mmol, 2.0 eq) at rt. The reaction mixture was heated at 140 °C in an oil-bath for 4 h. After completion of the reaction checked by TLC (R_{f}=0.3, PE/EA=3/1), water (20 mL) was added and adjusted to pH 6 with HCl (1 N). The mixture was extracted with EA (30 mL×2). The combined organic layer was washed with brine, dried over Na₂SO₄ and filtered. The filtrate was concentrated under reduced pressure. The residue was purified by column chromatography (100-200 silica gel, 50% EA in PE as eluent) to afford compound 3 (113 mg, 11%) as pale-yellow solid.

### Step 2: Synthesis of AB25882

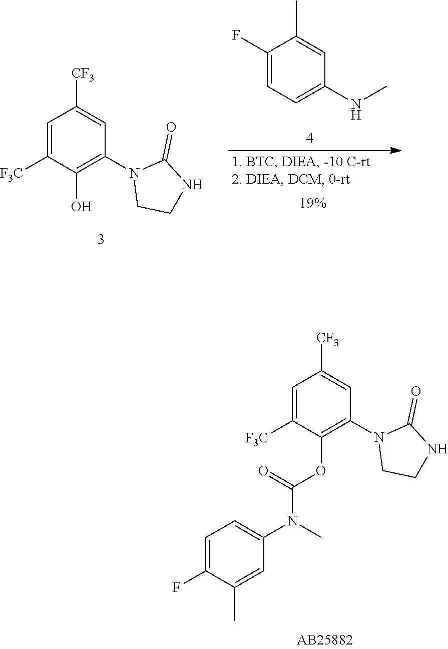

To a solution of compound 4 (113 mg, 0.35 mmol, 1.0 eq) in DCM (10 mL) were added DIEA (60 mg, 0.467 mmol, 1.5 eq) and a solution of triphosgene (117 mg, 0.395 mmol, 1.1 eq) in DCM (2 mL) at −10 °C. The reaction mixture was stirred for 1 h at rt under N2. The reaction solution was concentrated under reduced pressure to remove the solvent. Then DCM (3 mL) was added to above residue. The solution was used for next step.

### Step 3: Synthesis of 2-(2-oxoimidazolidin-1-yl)-4,6-bis(trifluoromethyl)phenyl N-(4-fluoro-3-methylphenyl)-N-methylcarbamate

To a solution of compound 3 (85 mg, 0.52 mmol, 1.5 eq) in DCM (5 mL) was added the above residue solution at 0 °C. The mixture was stirred for 1 h at rt under N2. After completion of the reaction monitored by TLC (R_{f}=0.5, PE/EA=2/1), HCl (1 N, 15 mL) was added. Then the mixture was extracted with DCM (20 mL×2). The combined organic layer was washed with brine, dried over Na₂SO₄ and filtered. The filtrate was concentrated under reduced pressure. The residue was purified by Prep-TLC and Prep-HPLC to give title compound (AB25882; 33.0 mg, 19%) as off-white solid.

¹H NMR (400 MHz, CDCl₃) δ 7.63 (s, 1H), 7.56 (s, 1H), 7.24 (s, 1H), 7.01-7.01 (d, 1H), 6.93-6.91 (t, 1H), 6.74-6.72 (t, 2H), 4.40 (t, 1H), 4.10-4.07 (t, 2H), 3.49-3.45 (m, 2H), 3.16 (s, 3H), 2.16 (s, 3H).

### Example 29

### 2-(2-oxoimidazolidin-1-yl)-4,6-bis(trifluoromethyl)phenyl N-methyl-N-{1-methyl-1H-pyrrolo[2,3-b]pyridin-6-yl}carbamate

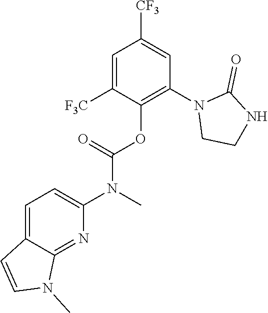 [SMILES: CN(C(=O)Oc1c(N2CCNC2=O)cc(C(F)(F)F)cc1C(F)(F)F)c1ccc2ccn(C)c2n1]

### Step 1: Synthesis of 2-(2,4-bis(trifluoromethyl)phenyl)-4,4,5,5-tetramethyl-1,3,2-dioxaborolane

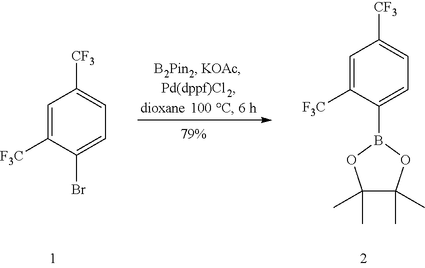

A solution of 1-bromo-2,4-bis(trifluoromethyl)benzene (1, 25.0 g, 85.3 mmol 1.0 eq) in 1,4 dioxane (300 mL) was purged with N2 in a sealed tube for 2 mins and then added bispinacolato diboron (43.4 g, 170 mmol, 2.0 eq), potassium acetate (16.8 g, 170 mmol) and Pd(dppf)Cl₂·DCM (2.4 g, 3 mmol, 0.04 eq) with stirring under inert atmosphere. The tightly closed sealed tube was heated to 100 °C in an oil bath for 16 h with stirring. Progress of the reaction was monitored by TLC (R_{f}=0.5, 10% EtOAc in Hexane) and LC-MS. After completion of the reaction, the reaction mixture was concentrated under reduced pressure. The residue was partitioned between ethyl acetate (200 mL) and water (150 mL). The organic layer was dried over Na₂SO₄, and concentrated under reduced pressure. The residue was purified by silica gel chromatography eluted with PE/EA=1/20 to give compound 2 (23.0 g, 79%).

### Step 2: Synthesis of 2,4-bis(trifluoromethyl)phenol

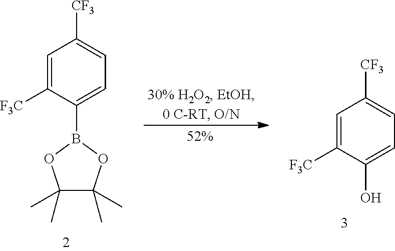 [SMILES: CC1(C)OB(c2ccc(C(F)(F)F)cc2C(F)(F)F)OC1(C)C ; "52%" ; "30% H2O2, EtOH,0 C-RT, O/N" ; Oc1ccc(C(F)(F)F)cc1C(F)(F)F]

To a solution of 2-(2,4-bis(trifluoromethyl)phenyl)-4,4,5,5-tetramethyl-1,3,2-dioxaborolane (2, 23.0 g, 67.6 mmol, 1.0 eq) in EtOH (300 mL) at 0 °C was added hydrogen peroxide 30% aq. solution (24 mL) under inert atmosphere with stirring. The reaction mixture was stirred at ambient temperature for 16 h. After completion of the reaction confirmed by TLC (R_{f}=0.2, 10% EtOAc in Hexane), the reaction mixture was cooled to 0 °C and quenched with aq. sodium metabisulfite solution (50 mL) followed by extraction with EtOAc (2×300 mL). The organic layer collected was washed with water followed by brine, dried over anhydrous Na₂SO₄, filtered and concentrated under reduced pressure to afford a crude. The crude was purified by column chromatography (100-200 silica gel, 0-20% EtOAc in hexane as eluent) to afford compound 3 (8.1 g, 52%) as pale yellow liquid.

### Step 3: Synthesis of 2-iodo-4,6-bis(trifluoromethyl)phenol

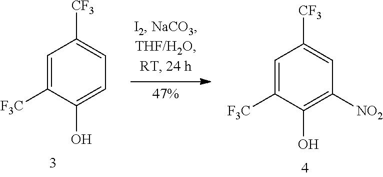 [SMILES: Oc1ccc(C(F)(F)F)cc1C(F)(F)F ; O=[N+]([O-])c1cc(C(F)(F)F)cc(C(F)(F)F)c1O]

A solution of 2,4-bis(trifluoromethyl)phenol (3, 8.0 g, 34.8 mol, 1.0 eq) in THF:H₂O (3:1, 160 mL) was cooled 0 °C in an ice bath with stirring. After 15 minutes, to the solution was added 12 (10.6 g, 41.7 mmol, 1.2 eq) followed by Na₂CO₃ (4.4 g, 41.5 mmol, 1.2 eq) under inert atmosphere with stirring. The reaction mixture was stirred at ambient temperature for 24 h. After completion of the reaction (TLC: R_{f}=0.35, 30% EtOAc in Hexane), the reaction mixture was cooled to 0 °C and then quenched with aq. sodium metabisulfite solution followed by extraction with EtOAc (2×150 mL). The organic layer collected was washed with water followed by brine, dried over anhydrous Na₂SO₄, filtered and concentrated under reduced pressure to afford a crude. The crude obtained was purified by column chromatography (100-200 silica gel, 0-5% EtOAc in hexane as eluent) to afford compound 4 (5.8 g, 47%) as pale-yellow solid.

### Step 4: Synthesis of 1-(2-hydroxy-3,5-bis(trifluoromethyl)phenyl)imidazolidine-2-one

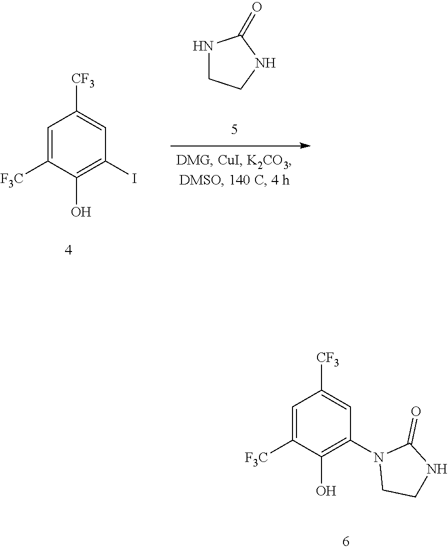

To a solution of compound 4 (1.2 g, 3.38 mmol, 1.0 eq) and compound 5 (581 mg, 6.76 mmol, 2.0 eq) in DMSO (10 mL) were added CuI (129 mg, 0.68 mmol, 0.2 eq), DMG (191 mg, 1.35 mmol, 0.4 eq) and K₂CO₃ (940 mg, 6.76 mmol, 2.0 eq) at rt. The reaction mixture was heated at 140 °C in an oil-bath for 4 h. After completion of the reaction by TLC (R_{f}=0.3, PE/EA=3/1), water (20 mL) was added and adjust to pH 6 with HCl (1 N). The mixture was extracted with EA (100 mL×2). The combined organic layer was washed with brine, dried over Na₂SO₄ and filtered. The filtration was concentrated under reduced pressure. The residue was purified by column chromatography (100-200 silica gel, 50% EA in PE as eluent) to afford compound 6 (113 mg, 11%) as pale-yellow solid.

### Step 5: Synthesis of tert-butyl (1H-pyrrolo[2,3-b]pyridine-6-yl)carbamate

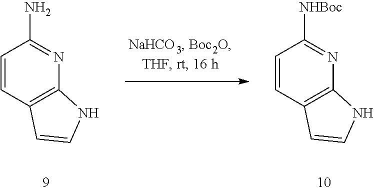

To a solution of compound 9 (560 mg, 4.2 mmol, 1.0 eq) in THF (10 mL) were added NaHCO₃ (sat. aq. 3 mL), Boc₂O (971 mg, 4.45 mmol, 1.05 eq) at rt. The reaction mixture was stirred for 16 h at rt. The mixture was extracted with EA (30 mL×2). The combined organic layer was washed with brine, dried over Na₂SO₄ and filtered. The filtrate was concentrated under reduced pressure. The residue was purified by column chromatography (100-200 silica gel, 20% EA in PE as eluent) to afford compound 10 (180 mg, 18%) as pale white solid.

### Step 6: Synthesis of tert-butyl methyl(1-methyl-1H-pyrrolo[2,3-b]pyridine-6-yl)carbamate

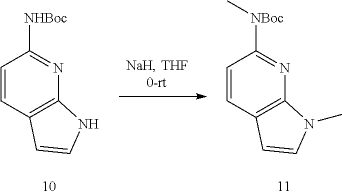

To a solution of compound 10 (180 mg, 0.77 mmol, 1.0 eq) in DMF (10 mL) was added 60% NaH (78 mg, 1.93 mmol, 2.5 eq) at 0 °C. The mixture was stirred for 30 mins at 0 °C. Then MeI (275 mg, 1.93 mmol, 2.5 eq) was added at 0 °C and stirred for 2 h. The mixture was extracted with EA (30 mL×2). The combined organic layer was washed with brine, dried over Na₂SO₄ and filtered. The filtration was concentrated under reduced pressure. The residue was purified by column chromatography (100-200 silica gel, 6% EA in PE as eluent) to afford compound 11 (183 mg, 90%) as pale-white solid.

### Step 7: Synthesis of N,1-dimethyl-1H-pyrrolo[2,3-b]pyridine-6-amine

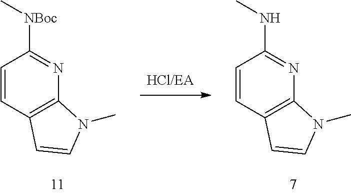

To a solution of 4 N HCl/EA (5 mL) was added compound 11 (183 mg, 0.70 mmol, 1.0 eq) at 0 °C. The reaction mixture was stirred for 2 h at rt. After completion of the reaction by TLC (R_{f}=0.6, PE/EA=3/1), the mixture was concentrated under reduced pressure to give compound 7 (120 mg, crude) as off-white solid.

### Step 8: Synthesis of AB25883

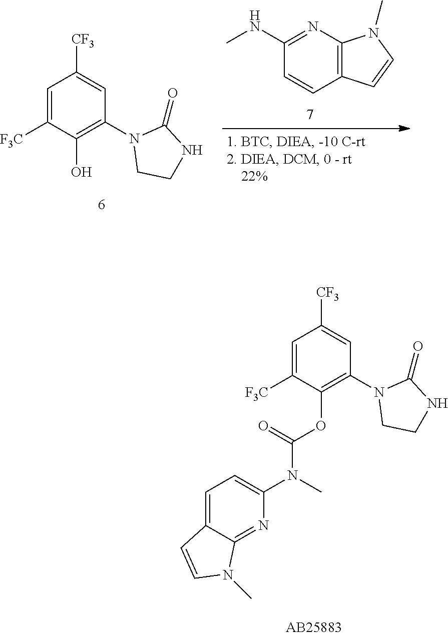 [SMILES: O=C1NCCN1c1cc(C(F)(F)F)cc(C(F)(F)F)c1O ; CNc1ccc2ccn(C)c2n1 ; CN(C(=O)Oc1c(N2CCNC2=O)cc(C(F)(F)F)cc1C(F)(F)F)c1ccc2ccn(C)c2n1]

To a solution of compound 6 (113 mg, 0.35 mmol, 1.0 eq) in DCM (10 mL) were added DIEA (60 mg, 0.467 mmol, 1.5 eq) and a solution of triphosgene (117 mg, 0.395 mmol, 1.1 eq, in DCM (2 mL) at −10 °C. The reaction mixture was stirred for 1 h at rt under N2. The reaction solution was concentrated under reduced pressure to remove the solvent. Then DCM (3 mL) was added to above residue. The solution was used for next step.

### Step 9: Synthesis of 2-(2-oxoimidazolidin-1-yl)-4,6-bis(trifluoromethyl)phenyl N-methyl-N-{1-methyl-1H-pyrrolo[2,3-b]pyridin-6-yl}carbamate

To a solution of compound 7 (85 mg, crude) in DCM (5 mL) at 0 °C was added the above residue solution. The mixture was stirred for 1 h at rt under N2. After completion of the reaction by TLC (R_{f}=0.5, PE/EA=2/1), HCl (1 M, 15 mL) was added. Then the mixture was extracted with DCM (20 mL×2). The combined organic layer was washed with brine, dried over Na₂SO₄ and filtered. The filtrate was concentrated under reduced pressure. The residue was purified by Prep-TLC and Prep-HPLC to give title compound (AB25883; 39.4 mg, 22%) as off-white solid.

¹H NMR (400 MHz, CDCl₃) δ 9.75 (t, 1H), 7.82-7.80 (d, 1H, J=8.0 Hz), 7.48 (s, 1H), 7.38 (s, 1H), 7.24 (s, 1H), 7.01 (d, 1H), 6.71-6.68 (d, 1H, J=12.0 Hz), 6.35-6.34 (d, 1H, J=4.0 Hz), 4.19-4.16 (t, 2H), 3.76-3.71 (m, 2H), 3.58-3.57 (s, 3H), 3.40-3.40 (s, 3H).

### Example 30

### 2-(3-(2-(((4-fluorophenyl)(methyl)carbamoyl)oxy)-3,5-bis(trifluoromethyl)phenyl)-2-oxoimidazolidin-1-yl)ethyl (2-(2-(2-(2-aminoehoxy)ethoxy)ethoxy)acetyl)sulfamate

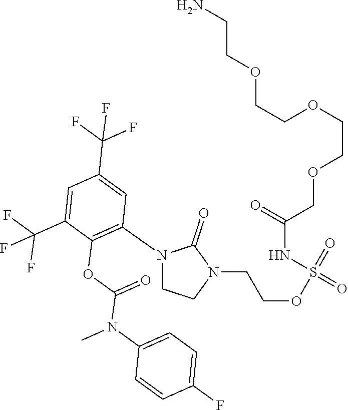 [SMILES: CN(C(=O)Oc1c(N2CCN(CCOS(=O)(=O)NC(=O)COCCOCCOCCN)C2=O)cc(C(F)(F)F)cc1C(F)(F)F)c1ccc(F)cc1]

### Step 1: Synthesis of 2-(2,4-bis(trifluoromethyl)phenyl)-4,4,5,5-tetramethyl-1,3,2-dioxaborolane

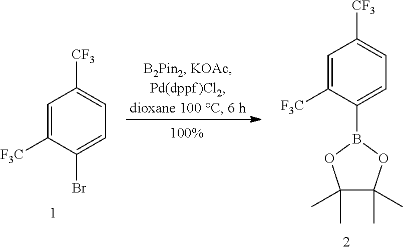 [SMILES: FC(F)(F)c1ccc(Br)c(C(F)(F)F)c1 ; "B2Pin2, KOAc,Pd(dppf)Cl2,dioxane 100 °C, 6 h" ; CC1(C)OB(c2ccc(C(F)(F)F)cc2C(F)(F)F)OC1(C)C]

A solution of 1-bromo-2,4-bis(trifluoromethyl)benzene (25.0 g, 85.3 mmol, 1.0 eq) in dioxane (300 mL) was purged with N2 and then added bispinacolatodiboron (43.4 g, 170.6 mmol, 2.0 eq), potassium acetate (16.8 g, 170.6 mmol, 2.0 eq) and Pd(dppf)Cl₂·DCM (2.4 g, 8.53 mmol, 0.1 eq) with stirring under inert atmosphere. The tightly closed sealed tube was heated to 100 °C in an oil bath for 16 h with stirring. Progress of the reaction was monitored by TLC (Rf=0.5, 10% EtOAc in Hexane). After completion of the reaction, the reaction mixture was concentrated under reduced pressure. The residue was partitioned between ethyl acetate (200 mL) and water (150 mL). The organic layer was dried over Na₂SO₄, and concentrated under reduced pressure. The residue was purified by silica gel chromatography (PE:EA, 1:20) to give compound 2 (30.0 g, 100%).

### Step 2: Synthesis of 2,4-bis(trifluoromethyl)phenol

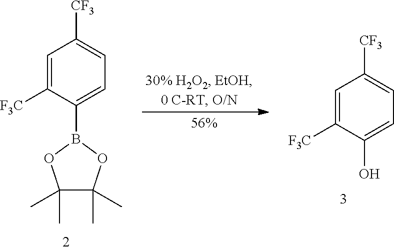 [SMILES: CC1(C)OB(c2ccc(C(F)(F)F)cc2C(F)(F)F)OC1(C)C ; "56%" ; "30% H2O2, EtOH,0 C-RT, O/N" ; Oc1ccc(C(F)(F)F)cc1C(F)(F)F]

To a solution of 2-(2,4-bis(trifluoromethyl)phenyl)-4,4,5,5-tetramethyl-1,3,2-dioxaborolane (30.0 g, 67.6 mmol, 1.0 eq) in ethanol (300 mL) at 0 °C was added hydrogen peroxide 30% aq. solution (32 mL) under inert atmosphere with stirring. The reaction mixture was stirred at ambient temperature for 16 h. After completion of the reaction confirmed by TLC (Rf 0.2, 10% EtOAc in PE), the reaction mixture was cooled to 0 °C and quenched with aq. sodium metabisulfite solution followed by extraction with EtOAc (2×200 mL). The organic layer was washed with water and brine, dried over anhydrous Na₂SO₄, filtered and concentrated under reduced pressure to afford a crude mixture. The crude obtained was purified by column chromatography (silica gel, 0-20% EtOAc in PE as eluent) to afford compound 3 (11.5 g, 56%) as pale-yellow liquid.

### Step 3: Synthesis of 2-iodo-4,6-bis(trifluoromethyl)phenol

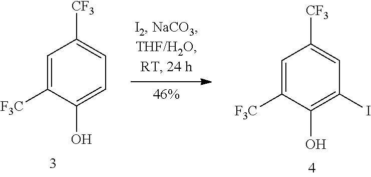 [SMILES: Oc1ccc(C(F)(F)F)cc1C(F)(F)F ; Oc1c(I)cc(C(F)(F)F)cc1C(F)(F)F]

A solution of 2,4-bis(trifluoromethyl)phenol (11.5 g, 50.0 mol, 1.0 eq) in THF:H₂O (3:1, 200 mL) was cooled at 0 °C in an ice bath with stirring. After 15 minutes, to the solution was added iodine (15.2 g, 60.0 mmol, 1.2 eq) followed by Na₂CO₃ (6.4 g, 60.0 mmol, 1.2 eq) under inert atmosphere with stirring. The reaction mixture was stirred at ambient temperature for 24 h. After completion of the reaction (TLC: R_{f} 0.35, 30% EtOAc in PE), the reaction mixture was cooled to 0 °C and then quenched with aq. sodium metabisulfite solution followed by extraction with EtOAc (2×150 mL). The organic layer was washed with water and brine, dried over anhydrous Na₂SO₄, filtered and concentrated under reduced pressure to afford a crude. The crude was purified by column chromatography (silica gel, 0-5% EtOAc in PE as eluent) to afford compound 4 (7.5 g, 46%) as pale-yellow solid.

### Step 4: Synthesis of 1-(2-hydroxy-3,5-bis(trifluoromethyl)phenyl)-3-(3-hydroxyethyl)imidazolidine-2-one

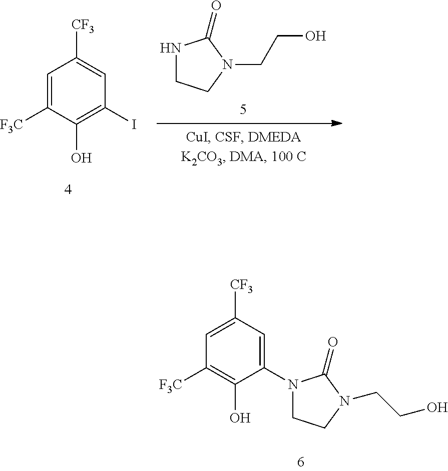 [SMILES: Oc1c(I)cc(C(F)(F)F)cc1C(F)(F)F ; O=C1NCCN1CCO ; O=C1N(CCO)CCN1c1cc(C(F)(F)F)cc(C(F)(F)F)c1O]

To a solution of compound 4 (2.0 g, 5.62 mmol, 1.0 eq) and compound 5 (1.1 g, 8.4 mmol, 1.5 eq) in DMA (5 mL) at rt was added CuI (534 mg, 2.81 mmol, 0.5 eq), N,N-dimethyl ethylenediamine (DMEDA, 490 mg, 5.62 mmol, 1.0 eq), K₂CO₃ (1.55 g, 11.2 mmol, 2.0 eq) and CsF (1.7 g, 11.2 mmol, 2.0 eq). The reaction mixture was heated at 100 °C for 4 h. After completion of the reaction by TLC (R_{f} 0.2, PE:EA=1:1), water (50 mL) was added and adjusted pH 6 with HCl (2 N). The mixture was extracted with EA (100 mL×2). The combined organic layer was washed with brine, dried over Na₂SO₄ and filtered. The filtration was concentrated under reduced pressure. The residue was purified by column chromatography (silica gel, 70% EA in PE as eluent) to afford compound 6 (1.38 g, 69%) as pale green solid.

### Step 5: Synthesis of 1-(2-benzyloxy-3,5-bis(trifluoromethyl)phenyl)-3-(3-hydroxyethyl)imidazolidine-2-one

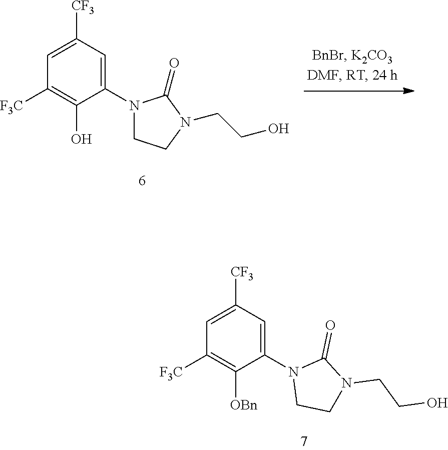 [SMILES: O=C1N(CCO)CCN1c1cc(C(F)(F)F)cc(C(F)(F)F)c1O ; O=C1N(CCO)CCN1c1cc(C(F)(F)F)cc(C(F)(F)F)c1OCc1ccccc1]

To a solution of compound 6 (2.15 g, 6.0 mmol, 1.0 eq) in DMF (10 mL) was added K₂CO₃ (1.09 g, 7.8 mmol, 1.3 eq) and benzyl bromide (1.23 g, 7.2 mmol, 1.2 eq). The reaction was stirred at 30 °C for 2 h. After completion of the reaction checked by TLC (R_{f}=0.3, PE:EA=1:1), the mixture was extracted with ethyl acetate (100 mL×2). The combined organic layer was washed with brine, dried over Na₂SO₄ and filtered. The filtrate was concentrated under the reduced pressure. The residue was purified by column chromatography (silica gel, 20% EA in PE as eluent) to afford compound 7 (1.89 g, 88%) as off-white solid.

### Step 6: Synthesis of 1-(2-(benzyloxy)-3,5-bis(trifluoromethyl)phenyl)-3-(2-tert-butyldimethylsilyl)oxy)ethyl)imidazolidine-2-one

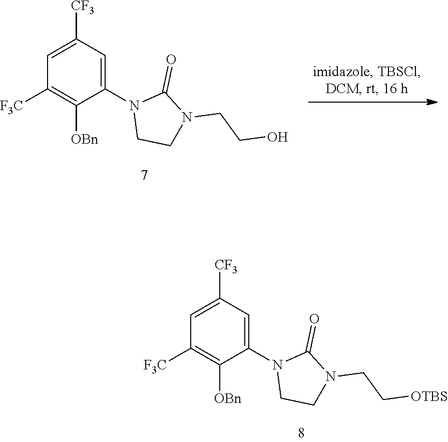 [SMILES: O=C1N(CCO)CCN1c1cc(C(F)(F)F)cc(C(F)(F)F)c1OCc1ccccc1 ; CC(C)(C)[Si](C)(C)OCCN1CCN(c2cc(C(F)(F)F)cc(C(F)(F)F)c2OCc2ccccc2)C1=O]

To a solution of compound 7 (1.89 g, 4.2 mmol, 1.0 eq) in DCM (10 mL) was added imidazole (580 mg, 8.43 mmol, 2.0 eq) and TBSCl (1.27 g, 8.43 mmol, 2.0 eq) at 0 °C. The reaction was stirred at rt for 16 h. After completion of the reaction checked by TLC (R_{f} 0.9, PE:EA=1:1), the mixture was extracted with EA (100 mL×2). The combined organic layer was washed with brine, dried over Na₂SO₄ and filtered. The filtrate was concentrated under the reduced pressure. The residue was purified by column chromatography (silica gel, 6% EA in PE as eluent) to afford compound 8 (2.15 g, 99%) as pale yellow oil.

### Step 7: Synthesis of 1-(2-(hydroxy)-3,5-bis(trifluoromethyl)phenyl)-3-(2-tert-butyldimethylsilyl)oxy)ethyl)imidazolidine-2-one

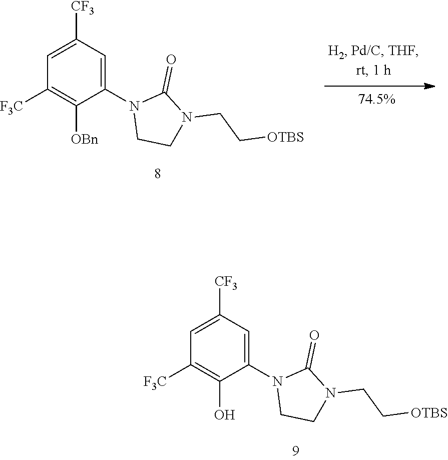 [SMILES: CC(C)(C)[Si](C)(C)OCCN1CCN(c2cc(C(F)(F)F)cc(C(F)(F)F)c2OCc2ccccc2)C1=O ; CC(C)(C)[Si](C)(C)OCCN1CCN(c2cc(C(F)(F)F)cc(C(F)(F)F)c2O)C1=O]

To a solution of compound 8 (2.12 g, 3.73 mmol, 1.0 eq) in THF (20 mL) at rt was added Pd/C (250 mg). The reaction was stirred for 1 h under H₂ atmosphere. After completion of the reaction by TLC (R_{f} 0.6, PE:EA=5:1), the mixture was filtered through a pad of Celite, and washed with EA (20 mL×2). The solution was concentrated to give compound 9 (1.58 g, 74%) as off-white solid.

### Step 8: Synthesis of 2-(3-(2-((tert-butyldimethylsilyl)oxy)ethyl)-2-oxoimidazolidin-1-yl)-4,6-bis(trifluoromethyl)phenyl methyl(phenyl)carbamate

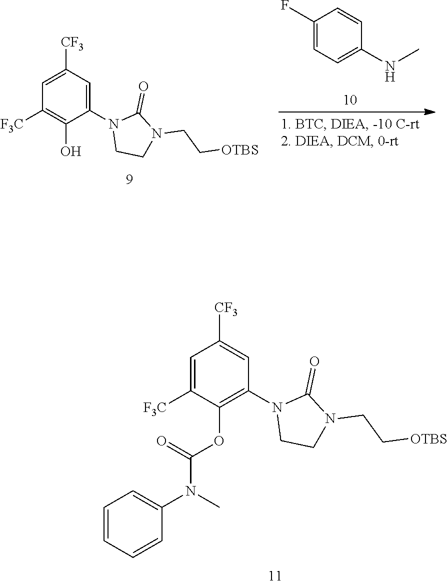

To a solution of compound 9 (500 mg, 1.05 mmol, 1.0 eq) in DCM (10 mL) at −10 °C was added DIEA (410 mg, 3.17 mmol, 3.0 eq) and triphosgene (141 mg, 0.476 mmol, 0.45 eq) in DCM (3 mL). The reaction mixture was stirred at rt under N2 for 10 min. The reaction mixture was concentrated under the reduced pressure. Then DCM (3 mL) added to the residue. The solution was used for next step.

To a solution of compound 10 (196 mg, 1.58 mmol, 1.5 eq) in DCM (5 mL) at 0 °C was added the solution prepared above. The mixture was stirred at rt under N2 for 1 h. After completion of the reaction monitored by TLC (R_{f} 0.5, PE:EA=5:1), HCl (1 M, 15 mL) was added. The mixture was extracted with DCM (20 mL×2). The combined organic layer was washed with brine, dried over Na₂SO₄ and filtered. The filtrate was concentrated under reduced pressure. The residue was purified with FCC to give compound 11 (370 mg, 74%) as off-yellow oil.

### Step 9: Synthesis of 2-(3-(2-hydroxyethyl)-2-oxoimidazolidin-1-yl)-4,6-bis(trifluoromethyl)phenyl (4-fluorophenyl)(methyl)carbamate

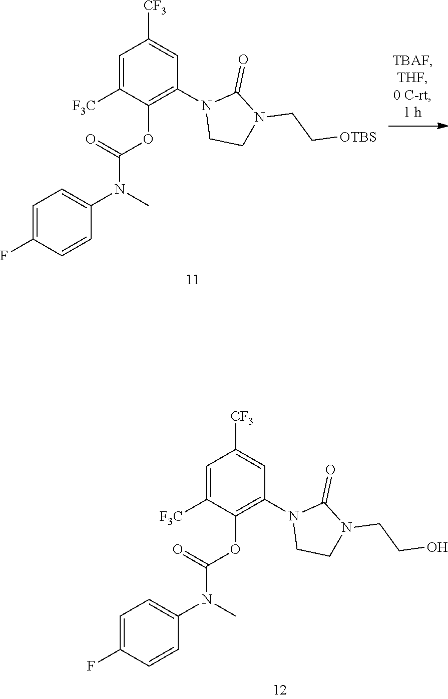 [SMILES: CN(C(=O)Oc1c(N2CCN(CCO[Si](C)(C)C(C)(C)C)C2=O)cc(C(F)(F)F)cc1C(F)(F)F)c1ccc(F)cc1 ; CN(C(=O)Oc1c(N2CCN(CCO)C2=O)cc(C(F)(F)F)cc1C(F)(F)F)c1ccc(F)cc1]

To a solution of compound 11 (350 mg, 0.561 mmol, 1.0 eq) in THF (10 mL) at 0 °C was added TBAF (1.68 mL, 1.68 mmol, 1.5 eq). The reaction was stirred at rt for 1 h. After completion of the reaction monitored by TLC (R_{f} 0.2, PE:EA=1:1), the mixture was extracted with EA (30 mL×2). The combined organic layer was washed with brine, dried over Na₂SO₄ and filtered. The filtrate was concentrated under reduced pressure. The residue was purified by column chromatography (silica gel, 70% EA in PE as eluent) to afford compound 12 (180 mg, 51%) as pale-yellow oil.

### Step 10: Synthesis of 2-(3-(2-(((4-fluorophenyl)(methyl)carbamoyl)oxy)-3,5-bis(trifluoromethyl)phenyl)-2-oxoimidazolidin-1-yl)ethyl sulfamate

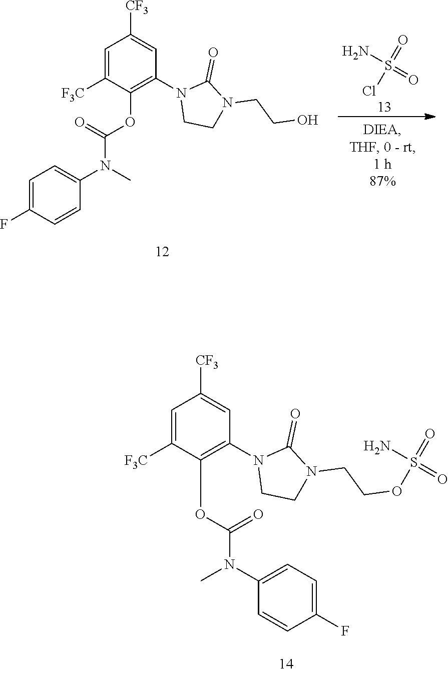 [SMILES: CN(C(=O)Oc1c(N2CCN(CCO)C2=O)cc(C(F)(F)F)cc1C(F)(F)F)c1ccc(F)cc1 ; NS(=O)(=O)Cl ; CN(C(=O)Oc1c(N2CCN(CCOS(N)(=O)=O)C2=O)cc(C(F)(F)F)cc1C(F)(F)F)c1ccc(F)cc1]

To a solution of compound 12 (100 mg, 0.196 mmol, 1.0 eq) in THF (10 mL) at 0 °C was added DIEA (76 mg, 0.59 mmol, 3.0 eq), and compound 13 (68 mg, 0.59 mmol, 3.0 eq). The reaction was stirred at rt for 1 h. After completion of the reaction by TLC (R_{f} 0.6, DCM:MeOH=10:1). The mixture was extracted with EA (30 mL×2). The combined organic layer was washed with brine, dried with Na₂SO₄ and filtered. The solution was concentrated under reduced pressure. The residue was purified by column chromatography (silica gel, 6% MeOH in DCM as eluent) to afford compound 14 (87 mg, 87%) as pale-yellow solid.

### Step 11: Synthesis of 2,5-dioxopyrrolidin-1-yl 2,2-dimethyl-4-oxo-3,8,11,14-tetraoxa-5-azahexadecan-16-oate

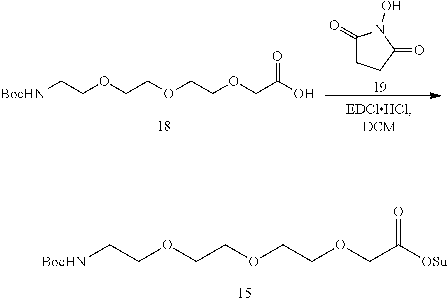

To a solution of compound 18 (500 mg, 1.63 mmol, 1.0 eq) in DCM (10 mL) at 0 °C was added EDCI (375 mg, 1.95 mmol, 1.2 eq) and compound 19 (225 mg, 1.95 mmol, 1.2 eq). The reaction was stirred at rt for 8 h. After completion of the reaction checked by TLC (R_{f}=0.8, DCM:MeOH=10:1), the mixture was extracted with EA (50 mL×2). The combined organic layer was washed with brine, dried over Na₂SO₄ and filtered. The solution was concentrated under reduced pressure to afford crude compound (800 mg, 100%) as pale-yellow solid.

### Step 12: Synthesis of 2-(3-(2-(((4-fluorophenyl)(methyl)carbamoyl)oxy)-3,5-bis(trifluoromethyl)phenyl)-2-oxoimidazolidin-1-yl)ethyl (2,2-dimethyl-4-oxo-3,8,11,14-tetraoxa-5-azahexadecan-16-oyl)sulfamate

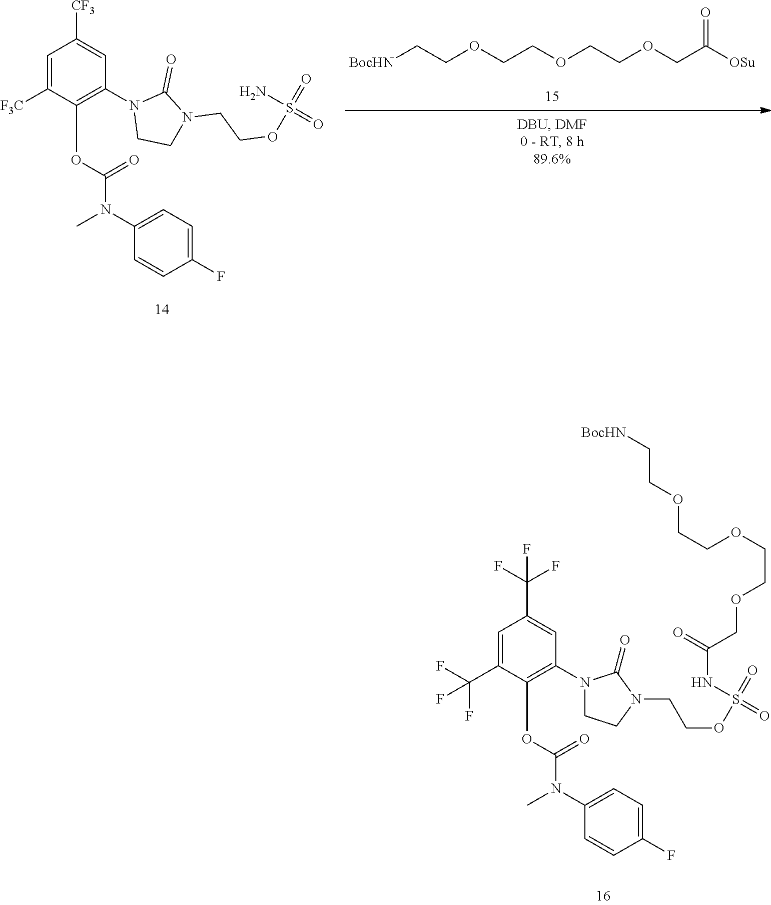

To a solution of compound 14 (87 mg, 0.148 mmol, 1.0 eq) in DMF (10 mL) at 0 °C was added DBU (28 mg, 0.177 mmol, 1.2 eq) and compound 15 (78 mg, 0.192 mmol, 1.3 eq). The reaction was stirred at rt for 8 h. After completion of the reaction monitored by TLC (R_{f}=0.7, DCM:MeOH=10:1), the mixture was extracted with EA (30 mL×2). The combined organic layer was washed with brine, dried over Na₂SO₄ and filtered. The filtrate was concentrated under reduced pressure to give a residue. The residue was purified by column chromatography (silica gel, 5% MeOH in DCM as eluent) to afford compound 16 (78 mg, 90%), as pale white solid.

### Step 13: Synthesis of 2-(3-(2-(((4-fluorophenyl)(methyl)carbamoyl)oxy)-3,5-bis(trifluoromethyl)phenyl)-2-oxoimidazolidin-1-yl)ethyl (2-(2-(2-(2-aminoehoxy)ethoxy)ethoxy)acetyl)sulfamate

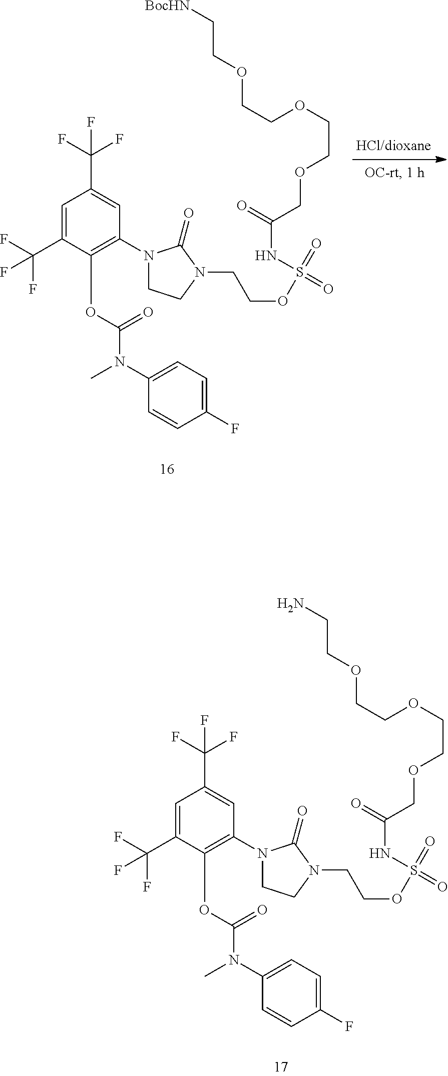 [SMILES: CN(C(=O)Oc1c(N2CCN(CCOS(=O)(=O)NC(=O)COCCOCCOCCNC(=O)OC(C)(C)C)C2=O)cc(C(F)(F)F)cc1C(F)(F)F)c1ccc(F)cc1 ; CN(C(=O)Oc1c(N2CCN(CCOS(=O)(=O)NC(=O)COCCOCCOCCN)C2=O)cc(C(F)(F)F)cc1C(F)(F)F)c1ccc(F)cc1]

To a solution of HCl/dioxane (5 mL, 4 M) at 0 °C was added compound 16 (78 mg, 0.08 mmol, 1.0 eq). The reaction was stirred at rt for 1 h. The reaction solution was concentrated under reduced pressure. The residue was purified by reversed-phase prep-HPLC to afford title compound (17; 28.4 mg, 36%) as off-white solid.

¹H NMR (400 MHz, CDCl₃): δ 7.91 (s, 1H), 7.71 (d, 1H, J=8.0 Hz), 7.41 (m, 2H), 7.10-7.06 (t, 2H), 4.28 (s, 2H), 3.97 (s, 2H), 3.59-3.46 (m, 16H), 3.34 (s, 3H), 3.03 (s, 2H).

### Example 31

### 2,4-bis(trifluoromethyl)-6-(2-oxoimidazolidin-1-yl)phenyl cyclopropyl4-fluorophenylcarbamate

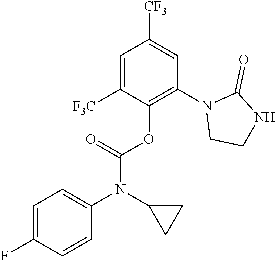 [SMILES: O=C1NCCN1c1cc(C(F)(F)F)cc(C(F)(F)F)c1OC(=O)N(c1ccc(F)cc1)C1CC1]

### Step 1: Synthesis of 2-(2,4-bis(trifluoromethyl)phenyl)-4,4,5,5-tetramethyl-1,3,2-dioxaborolane

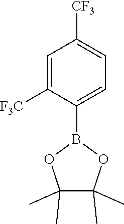 [SMILES: CC1(C)OB(c2ccc(C(F)(F)F)cc2C(F)(F)F)OC1(C)C]

A solution of 1-bromo-2,4-bis(trifluoromethyl)benzene (3 g, 0.01 mol) in 1,4 dioxane (90 mL) was purged with N2 in a sealed tube for 30 min and then added bispinacolato diboron (5.07 g, 0.02 mol), potassium acetate (1.96 g, 0.02 mol) and Pd(dppf)Cl₂·DCM (0.816 g, 0.001 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4×75 mL); washings collected was concentrated under reduced pressure to afford the yellow sticky solid (crude 6 g, Quantitative). The product was taken as such to next step without further purification.

### Step 2: Synthesis of 2,4-bis(trifluoromethyl)phenol

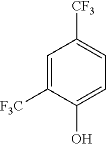 [SMILES: Oc1ccc(C(F)(F)F)cc1C(F)(F)F]

To a cold (0 °C) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×100 mL). The organic layer collected dried over anhydrous Na₂SO₄, concentrated under reduced pressure to afford the crude. The crude was purified by column chromatography (100-200 silica gel, 10-15% EtOAc in Hexane as eluent) to afford the title compound as pale yellow liquid (1.5 g, 65% yield).

### Step 3: Synthesis of 2-iodo-4,6-bis(trifluoromethyl)phenol

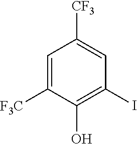 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20:6 mL) was cooled 0 °C in an ice bath with stirring. After 15 minutes, was added 12 (1.43 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 30% EtOAc in Hexane followed by 30% DCM in Hexane), the reaction mixture was cooled to 0 °C and then quenched with aq. sodium metabisulfite solution followed by extraction with EtOAc (2×100 mL). The organic layer collected dried over anhydrous Na₂SO₄, filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 4: Synthesis of cyclopropyl(4-fluorophenyl)carbamic chloride

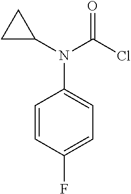 [SMILES: O=C(Cl)N(c1ccc(F)cc1)C1CC1]

To a cold (0 °C) solution of triphosgene (0.29 g, 0.0009 mol) in DCM (12 mL) added a solution of N-cyclopropyl-4-fluoroaniline (0.3 g, 0.00198 mol) and pyridine (0.313 g, 0.32 mL, 0.0039 mol) dropwise for over a period of 10 min. After that continued stirring at RT for 16 h. Progress of the reaction was monitored by TLC (R_{f} −0.4, 10% EtOAc in Hexane). After completion of the reaction, quenched the reaction mixture with 1M aq·HCl (10 mL) and then extracted with DCM (2×25 mL). The DCM layer separated was dried over anhydrous Na₂SO₄, filtered, and concentrated under reduced pressure to afford the title compound as yellow liquid (0.26 g, 63%).

### Step 5: Synthesis of 2-iodo-4,6-bis(trifluoromethyl)phenylcyclopropyl(4-fluorophenyl)carbamate

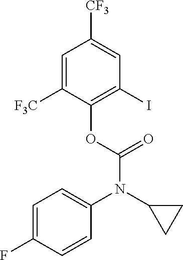 [SMILES: O=C(Oc1c(I)cc(C(F)(F)F)cc1C(F)(F)F)N(c1ccc(F)cc1)C1CC1]

To a stirred solution of 2-iodo-4,6-bis(trifluoromethyl)phenol, (0.2 g, 0.00056 mol) in Pyridine (4 mL) at RT added cyclopropyl(4-fluorophenyl)carbamic chloride (0.14 g, 0.00067 mol) and continued stirring at 80 °C for 4 hours. After completion of the reaction was confirmed by TLC (R_{f} −0.3, 10% DCM in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as colourless liquid (0.2 g, 69%).

### Step 6: Synthesis of 2-(2-oxoimidazolidin-1-yl)-4,6-bis(trifluoromethyl)phenyl cyclopropyl(4-fluorophenyl)carbamate

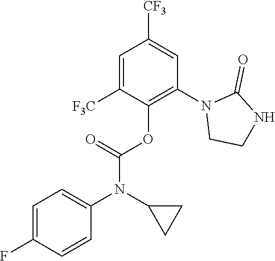 [SMILES: O=C1NCCN1c1cc(C(F)(F)F)cc(C(F)(F)F)c1OC(=O)N(c1ccc(F)cc1)C1CC1]

2-iodo-4,6-bis(trifluoromethyl)phenylcyclopropyl(4-fluorophenyl)carbamate (0.2 g, 0.00037 mol), 2-imidazilidinone (0.064 g, 0.0007 mol), copper (1)-iodide (0.035 g, 0.00018 mol), N,N′-dimethylethylenediamine (0.04 mL, 0.00037 mol), caesium fluoride (0.11 g, 0.00075 mol) and potassium carbonate (0.1 g, 0.00075 mol) were suspended in 1,4-dioxane priorly purged with N2 for 30 minutes (16 mL). The reaction mixture was heated at 100 °C in an oil-bath for 8 h. After completion of the reaction by TLC (R_{f} 0.3, 5% MeOH in DCM) the reaction mixture was concentrated under reduced pressure. The residue obtained was purified by column chromatography (100-200 silica gel, 50-60% EtOAc in Hexane as eluent) to afford the impure compound. The impure compound was re-purified by preparative TLC (solid phase: merck, 20×20 cm, silicagel 60 GF₂₅₄, 1 mm, PLC glass plate, 2% MeOH in DCM as eluent) to afford pure title compound as white solid (36 mg, 10%).

¹H NMR (400 MHz, Trifluoroacetic acid-D) δ 7.90 (m, 2H), 7.14 (d, J=30.1 Hz, 2H), 7.01 (t, J=8.2 Hz, 2H), 3.97 (d, J=5.9 Hz, 2H), 3.79 (t, J=8.1 Hz, 2H), 3.21 (d, J=49.2 Hz, 1H), 0.89 (m, 4H); MS(ESI): m/z 492.35 (M+H)⁺.

### Example 32

### 2-(3-(2-(difluoromethoxy)ethyl)-2-oxoimidazolidin-1-yl)-4,6-bis(trifluoromethyl)phenyl N-(4-fluorophenyl)-N-methylcarbamate

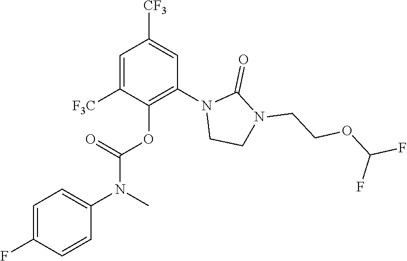 [SMILES: CN(C(=O)Oc1c(N2CCN(CCOC(F)F)C2=O)cc(C(F)(F)F)cc1C(F)(F)F)c1ccc(F)cc1]

### Step 1: Synthesis of 2-(2,4-bis(trifluoromethyl)phenyl)-4,4,5,5-tetramethyl-1,3,2-dioxaborolane

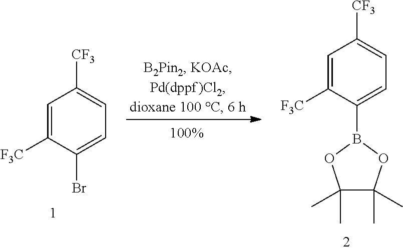 [SMILES: FC(F)(F)c1ccc(Br)c(C(F)(F)F)c1 ; "B2Pin2, KOAc,Pd(dppf)Cl2,dioxane 100 °C, 6 h" ; CC1(C)OB(c2ccc(C(F)(F)F)cc2C(F)(F)F)OC1(C)C]

A solution of 1-bromo-2,4-bis(trifluoromethyl)benzene (25.0 g, 85.3 mmol, 1.0 eq) in dioxane (300 mL) was purged with N2 and then added bispinacolatodiboron (43.4 g, 170.6 mmol, 2.0 eq), potassium acetate (16.8 g, 170.6 mmol, 2.0 eq) and Pd(dppf)Cl₂·DCM (2.4 g, 8.53 mmol, 0.1 eq) with stirring under inert atmosphere. The tightly closed sealed tube was heated to 100 °C in an oil bath for 16 h with stirring. Progress of the reaction was monitored by TLC (Rf=0.5, 10% EtOAc in Hexane). After completion of the reaction, the reaction mixture was concentrated under reduced pressure. The residue was partitioned between ethyl acetate (200 mL) and water (150 mL). The organic layer was dried over Na₂SO₄, and concentrated under reduced pressure. The residue was purified by silica gel chromatography (PE:EA, 1:20) to give compound 2 (30.0 g, 100%).

### Step 2: Synthesis of 2,4-bis(trifluoromethyl)phenol

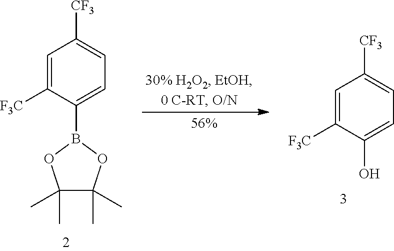 [SMILES: CC1(C)OB(c2ccc(C(F)(F)F)cc2C(F)(F)F)OC1(C)C ; "56%" ; "30% H2O2, EtOH,0 C-RT, O/N" ; Oc1ccc(C(F)(F)F)cc1C(F)(F)F]

To a solution of 2-(2,4-bis(trifluoromethyl)phenyl)-4,4,5,5-tetramethyl-1,3,2-dioxaborolane (30.0 g, 67.6 mmol, 1.0 eq) in ethanol (300 mL) at 0 °C was added hydrogen peroxide 30% aq. solution (32 mL) under inert atmosphere with stirring. The reaction mixture was stirred at ambient temperature for 16 h. After completion of the reaction confirmed by TLC (Rf 0.2, 10% EtOAc in PE), the reaction mixture was cooled to 0 °C and quenched with aq. sodium metabisulfite solution followed by extraction with EtOAc (2×200 mL). The organic layer was washed with water and brine, dried over anhydrous Na₂SO₄, filtered and concentrated under reduced pressure to afford a crude mixture. The crude obtained was purified by column chromatography (silica gel, 0-20% EtOAc in PE as eluent) to afford compound 3 (11.5 g, 56%) as pale-yellow liquid.

### Step 3: Synthesis of 2-iodo-4,6-bis(trifluoromethyl)phenol

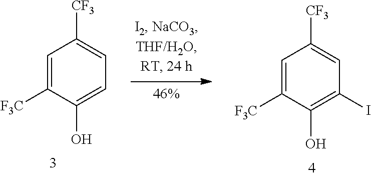 [SMILES: Oc1ccc(C(F)(F)F)cc1C(F)(F)F ; Oc1c(I)cc(C(F)(F)F)cc1C(F)(F)F]

A solution of 2,4-bis(trifluoromethyl)phenol (11.5 g, 50.0 mol, 1.0 eq) in THF:H₂O (3:1, 200 mL) was cooled at 0 °C in an ice bath with stirring. After 15 minutes, to the solution was added iodine (15.2 g, 60.0 mmol, 1.2 eq) followed by Na₂CO₃ (6.4 g, 60.0 mmol, 1.2 eq) under inert atmosphere with stirring. The reaction mixture was stirred at ambient temperature for 24 h. After completion of the reaction (TLC: R_{f} 0.35, 30% EtOAc in PE), the reaction mixture was cooled to 0 °C and then quenched with aq. sodium metabisulfite solution followed by extraction with EtOAc (2×150 mL). The organic layer was washed with water and brine, dried over anhydrous Na₂SO₄, filtered and concentrated under reduced pressure to afford a crude. The crude was purified by column chromatography (silica gel, 0-5% EtOAc in PE as eluent) to afford compound 4 (7.5 g, 46%) as pale-yellow solid.

### Step 4: Synthesis of 1-(2-(difluoromethoxy)ethyl)imidazolidine-2-one

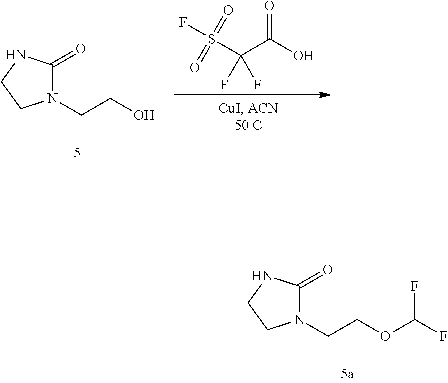 [SMILES: O=C1NCCN1CCO ; O=C(O)C(F)(F)S(=O)(=O)F ; O=C1NCCN1CCOC(F)F]

To a solution of compound 5 (3.5 g, 26.7 mmol, 1.0 eq) in acetonitrile (10 mL) at rt was added CuI (1.53 g, 8.02 mmol, 0.3 eq), compound 9 (5.0 g, 28.1 mmol, 1.1 eq). The reaction was stirred at 50 °C for 4 h. After completion of the reaction monitored by TLC (R_{f} 0.3, PE:EA=1:1), the mixture was extracted with EA (50 mL×2). The combined organic layers were washed with water and brine, dried over Na₂SO₄ and filtered. The solution was concentrated under reduced pressure to give a residue. The residue was purified by column chromatography (silica gel, 40% EA in PE as eluent) to afford compound 5a (550 mg, 16%) as pale white solid.

### Step 5: Synthesis of 1-(2-(difluoromethoxy)ethyl)-3-(2-hydroxy-3,5-bis(trifluoromethyl)phenyl)imidazolidine-2-one

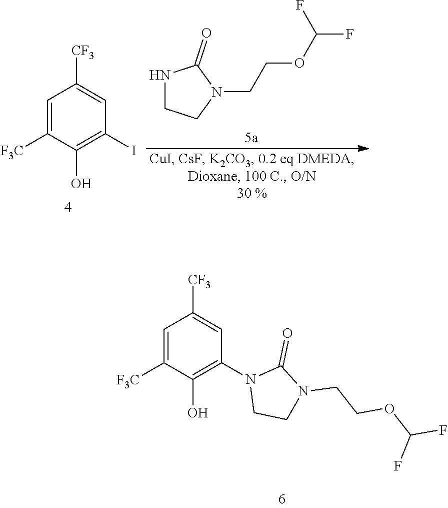 [SMILES: Oc1c(I)cc(C(F)(F)F)cc1C(F)(F)F ; O=C1NCCN1CCOC(F)F ; O=C1N(CCOC(F)F)CCN1c1cc(C(F)(F)F)cc(C(F)(F)F)c1O]

To a solution of compound 4 (215 mg, 0.6 mmol, 1.0 eq) and compound 5a (131 mg, 0.727 mmol, 1.2 eq) in DMA (5 mL) at rt was added CuI (57 mg, 0.3 mmol, 0.5 eq), DMEDA (53 mg, 0.6 mmol, 1.0 eq), K₂CO₃ (167 mg, 1.2 mmol, 2.0 eq) and CsF (182 g, 1.2 mmol, 2.0 eq). The reaction mixture was heated at 100 °C in an oil-bath for 4 h. After being cooled to rt, water (50 mL) was added and adjusted to pH 6 with HCl (2N). The mixture was extracted with ethyl acetate (50 mL×2). The combined organic layer was washed with brine, dried over Na₂SO₄ and filtered. The solution was concentrated under reduced pressure. The residue was purified by column chromatography (silica gel, 30% EA in PE as eluent) to afford compound 6 (65 mg, 30%) as light-yellow solid.

### Step 6: Synthesis of 2-(3-(2-(difluoromethoxy)ethyl)-2-oxoimidazolidin-1-yl)-4,6-bis(trifluoromethyl)phenyl (4-fluorophenyl)(methyl)carbamate

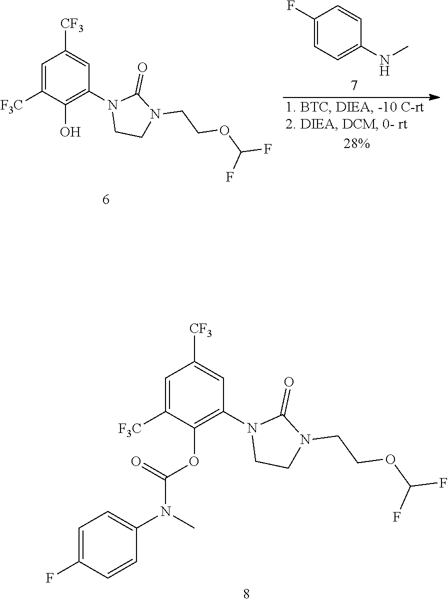

To a solution of compound 6 (100 mg, 0.245 mmol, 1.0 eq) in DCM (10 mL) at −10 °C was added DIEA (48 mg, 0.367 mmol, 1.5 eq) and triphosgene (81 mg, 0.269 mmol, 1.1 eq) in DCM (2 mL). The reaction mixture was stirred at rt under N2 for 1 h. The reaction solution was concentrated under reduced pressure to remove the solvent, and dissolved in DCM (3 mL). The solution was used for next step without further purification.

### Step 7

To a solution of compound 7 (32 mg, 0.25 mmol, 1.2 eq) in DCM (5 mL) at 0 °C was added the solution prepared above. The mixture was stirred at rt under N2 for 1 h. After completion of the reaction monitored by TLC (R_{f} 0.5, PE:EA=2:1), HCl (1 N, 15 mL) was added. Then the mixture was extracted with DCM (20 mL×2). The combined organic layer was washed with brine, dried over sodium sulfate and filtered. The filtrate was concentrated under reduced pressure to give a residue. The residue was purified Prep-HPLC to give pure title compound (8; 29 mg, 28%) as off-white solid.

¹H NMR (400 MHz, CDCl₃): δ 7.83-7.73 (m, 2H), 7.27 (s, 2H), 7.07 (t, 2H), 6.42-6.05 (t, 1H), 4.04 (s, 2H), 3.86 (s, 2H), 3.65-3.56 (t, 4H), 3.47 (s, 1H), 3.32 (s, 2H).

### Example 33

2,4-bis(trifluoromethyl)-6-(2-oxoimidazolidin-1-yl)phenyl 2-(cyanomethyl)phenylmethylcarbamate

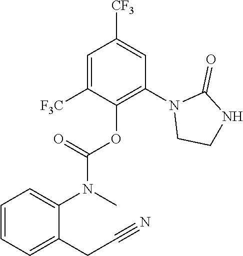 [SMILES: CN(C(=O)Oc1c(N2CCNC2=O)cc(C(F)(F)F)cc1C(F)(F)F)c1ccccc1CC#N]

### Step 1: Synthesis of 2-(2,4-bis(trifluoromethyl)phenyl)-4,4,5,5-tetramethyl-1,3,2-dioxaborolane

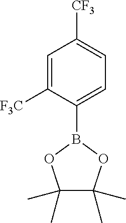 [SMILES: CC1(C)OB(c2ccc(C(F)(F)F)cc2C(F)(F)F)OC1(C)C]

A solution of 1-bromo-2,4-bis(trifluoromethyl)benzene (3 g, 0.01 mol) in 1,4 dioxane (90 mL) was purged with N2 in a sealed tube for 30 min and then added bispinacolato diboron (5.07 g, 0.02 mol), potassium acetate (1.96 g, 0.02 mol) and Pd(dppf)Cl₂·DCM (0.816 g, 0.001 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4×75 mL); washings collected was concentrated under reduced pressure to afford the yellow sticky solid (crude 6 g, Quantitative). The product was taken as such to next step without further purification.

### Step 2: Synthesis of 2,4-bis(trifluoromethyl)phenol

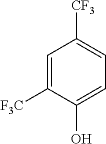 [SMILES: Oc1ccc(C(F)(F)F)cc1C(F)(F)F]

To a cold (0 °C) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×100 mL). The organic layer collected dried over anhydrous Na₂SO₄, concentrated under reduced pressure to afford the crude. The crude was purified by column chromatography (100-200 silica gel, 10-15% EtOAc in Hexane as eluent) to afford the title compound as pale yellow liquid (1.5 g, 65% yield).

### Step 3: Synthesis of 2-iodo-4,6-bis(trifluoromethyl)phenol

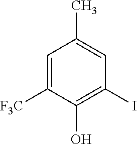

A solution of 2,4-bis(trifluoromethyl)phenol (1.0 g, 0.0043 mol) in THF:H₂O (3:1, 20:6 mL) was cooled 0 °C in an ice bath with stirring. After 15 minutes, was added 12 (1.43 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 30% EtOAc in Hexane followed by 30% DCM in Hexane), the reaction mixture was cooled to 0 °C and then quenched with aq. sodium metabisulfite solution followed by extraction with EtOAc (2×100 mL). The organic layer collected dried over anhydrous Na₂SO₄, filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 4: Synthesis of (2-(cyanomethyl)phenyl)(methyl)carbamic chloride

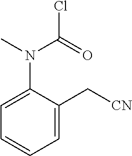

To a cold (0 °C) solution of triphosgene (0.081 g, 0.00027 mol) in DCM (30 mL) added a solution of 2-(2-(methylamino)phenyl)acetonitrile (0.08 g, 0.00054 mol) and pyridine (0.085 g, 0.087 mL, 0.001 mol) dropwise for over a period of 10 min. After that continued stirring at RT for 20 h. Progress of the reaction was monitored by TLC (R_{f} −0.4, 50% DCM in Hexane). After completion of the reaction, quenched the reaction mixture with 1M aq·HCl (10 mL) and then extracted with DCM (2×25 mL). The DCM layer separated was dried over anhydrous Na₂SO₄, filtered, and concentrated under reduced pressure to afford the title compound as yellow liquid (0.07 g, 62%).

### Step 5: Synthesis of 2-iodo-4,6-bis(trifluoromethyl)phenyl (2-(cyanomethyl)phenyl)(methyl)carbamate

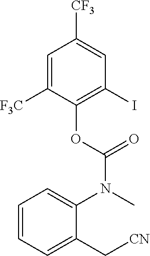 [SMILES: CN(C(=O)Oc1c(I)cc(C(F)(F)F)cc1C(F)(F)F)c1ccccc1CC#N]

To a stirred solution of 2-iodo-4,6-bis(trifluoromethyl)phenol, (0.05 g, 0.00014 mol) in Pyridine (1 mL) at RT added (2-(cyanomethyl)phenyl)(methyl)carbamic chloride (0.035 g, 0.00017 mol) and continued stirring at 80 °C for 16 hours. After completion of the reaction was confirmed by TLC (R_{f} −0.7, 30% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 3-6% EtOAc in Hexane as eluent) to afford the title compound as yellow liquid (0.02 g, 28%).

### Step 6: Synthesis of 2-(2-oxoimidazolidin-1-yl)-4,6-bis(trifluoromethyl)phenyl (2-(cyanomethyl)phenyl)(methyl)carbamate

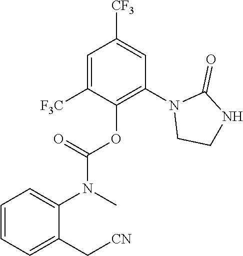 [SMILES: CN(C(=O)Oc1c(N2CCNC2=O)cc(C(F)(F)F)cc1C(F)(F)F)c1ccccc1CC#N]

2-iodo-4,6-bis(trifluoromethyl)phenyl (2-(cyanomethyl)phenyl)(methyl)carbamate (0.25 g, 0.00047 mol), 2-imidazilidinone (0.081 g, 0.00095 mol), copper (I)-iodide (0.045 g, 0.00024 mol), N,N′-dimethylethylenediamine (0.05 mL, 0.00047 mol), caesium fluoride (0.14 g, 0.00095 mol) and potassium carbonate (0.13 g, 0.00095 mol) were suspended in 1,4-dioxane priorly purged with N2 for 30 minutes (20 mL). The reaction mixture was heated at 100 °C in an oil-bath for 6 h. After completion of the reaction by TLC (R_{f} 0.4, 30% EtOAc in Hexane) the reaction mixture was concentrated under reduced pressure. The residue obtained was purified by column chromatography (100-200 silica gel, 50-60% EtOAc in Hexane as eluent) to afford the impure compound. The impure compound was re-purified by preparative TLC (solid phase: merck, 20×20 cm, silicagel 60 GF₂₅₄, 1 mm, PLC glass plate, 60% EtOAc in Hexane as eluent) to afford the pure title compound as pale yellow solid (22 mg, 10%).

¹H NMR (400 MHz, Trifluoroacetic acid-D) δ 7.92 (m, 2H), 7.40 (m, 6H), 3.96 (m, 6H), 3.47 (d, J=66.1 Hz, 3H); MS(ESI): m/z 487.30 (M+H)⁺.

### Example 34

### 2-(3-(3-hydroxycyclobutyl)-2-oxoimidazolidin-1-yl)-4,6-bis(trifluoromethyl)phenyl (4-Fluorophenyl)(methyl)carbamate

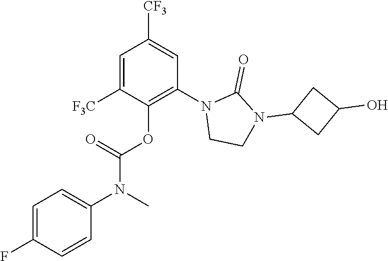 [SMILES: CN(C(=O)Oc1c(N2CCN(C3CC(O)C3)C2=O)cc(C(F)(F)F)cc1C(F)(F)F)c1ccc(F)cc1]

### Step 1: Synthesis of 1-(2-hydroxy-3,5-bis(trifluoromethyl)phenyl)imidazolidine-2-one

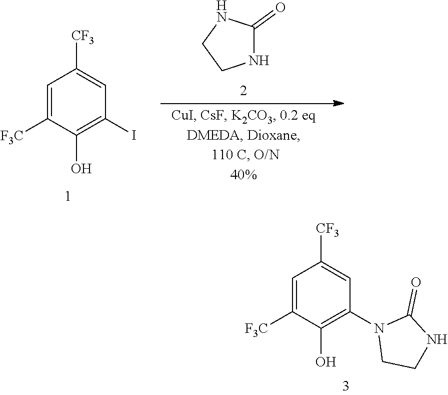 [SMILES: Oc1c(I)cc(C(F)(F)F)cc1C(F)(F)F ; O=C1NCCN1 ; O=C1NCCN1c1cc(C(F)(F)F)cc(C(F)(F)F)c1O]

To a solution of compound 1 (1.0 g, 2.81 mmol, 1.0 eq) and compound 2 (474 mg, 5.63 mmol, 2.0 eq) in DMA (10 mL) at rt was added CuI (270 mg, 1.4 mmol, 0.5 eq), DMEDA (249 mg, 2.81 mmol, 1.0 eq), K₂CO₃ (782 mg, 5.62 mmol, 2.0 eq) and CsF (855 mg, 5.62 mmol, 2.0 eq). The reaction mixture was heated at 100 °C in an oil-bath for 4 h. After completion of the reaction monitored by TLC (R_{f} 0.3, PE:EA=2:1) and LC-MS, water (20 mL) was added and the solution was adjusted to pH6 with HCl (2 N). The mixture was extracted with ethyl acetate (30 mL×2). The combined organic layer was washed with brine, dried over Na₂SO₄ and filtered. The filtrate was concentrated under reduced pressure. The residue was purified by column chromatography (100-200 silica gel, 50% EA in PE as eluent) to afford compound 3 (360 mg, 40%) as pale-yellow solid.

### Step 2: Synthesis of 2-(2-oxoimidazolidin-1-yl)-4,6-bis(trifluoromethyl)phenyl (4-fluorophenyl)(methyl)carbamate

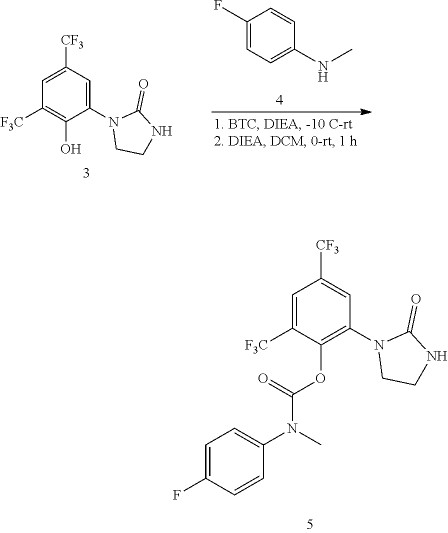

To a solution of compound 3 (220 mg, 0.7 mmol, 1.0 eq) in DCM (10 mL) at −10 °C was added DIEA (136 mg, 1.05 mmol, 1.5 eq), and triphosgene (93 mg, 0.32 mmol, 0.45 eq) in DCM (2 mL). The reaction mixture was stirred at rt under N2 for 1 h. The reaction solution was concentrated under reduced pressure to remove the solvent. Then DCM (3 mL) added to above residue. The solution was used for next step.

To a solution compound 4 (131 mg, 1.05 mmol, 1.5 eq) in DCM (5 mL) at 0 °C was added the above solution. The mixture was stirred at rt under N2 for 1 h. After completion of the reaction monitored by TLC (R_{f} 0.5, PE:EA=2:1) and LC-MS, HCl (1 N, 15 mL) was added. The mixture was extracted with DCM (20 mL×2). The combined organic layer was washed with brine, dried over Na₂SO₄ and filtered. The filtrate was concentrated under reduced pressure. The residue was purified by Prep-TLC and Prep-HPLC to give compound 5 (100 mg, 33%) as off-white solid.

### Step 3: Synthesis of 2-(2-oxo-3-(3-oxocyclobutyl)imidazolidine-1-yl)-4,6-bis(trifluoromethyl)phenyl (4-fluorophenyl)(methyl)carbamate

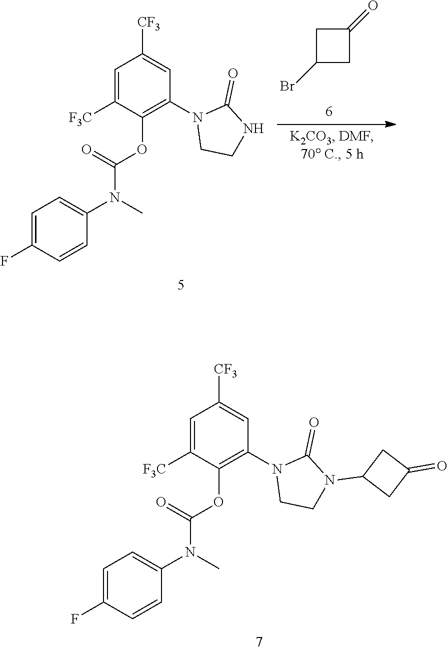 [SMILES: CN(C(=O)Oc1c(N2CCNC2=O)cc(C(F)(F)F)cc1C(F)(F)F)c1ccc(F)cc1 ; O=C1CC(Br)C1 ; CN(C(=O)Oc1c(N2CCN(C3CC(=O)C3)C2=O)cc(C(F)(F)F)cc1C(F)(F)F)c1ccc(F)cc1]

To a solution of compound 5 (100 mg, 0.215 mmol, 1.0 eq) and compound 6 (64 mg, 0.43 mmol, 2.0 eq) in DMF (3 mL) was added K₂CO₃ (60 mg, 0.43 mmol, 2.0 eq) at rt. The reaction mixture was heated at 70 °C in an oil-bath for 5 h. After completion of the reaction by TLC (R_{f} 0.6, PE:EA=2:1), the mixture was diluted with ethyl acetate (60 mL), washed with brine, dried over Na₂SO₄ and filtered. The solution was concentrated under reduced pressure. The residue was purified by column chromatography (100-200 silica gel, 40% EA in PE as eluent) to afford compound 7 (95 mg, 82%) as pale-yellow solid.

### Step 4: Synthesis of AB25948

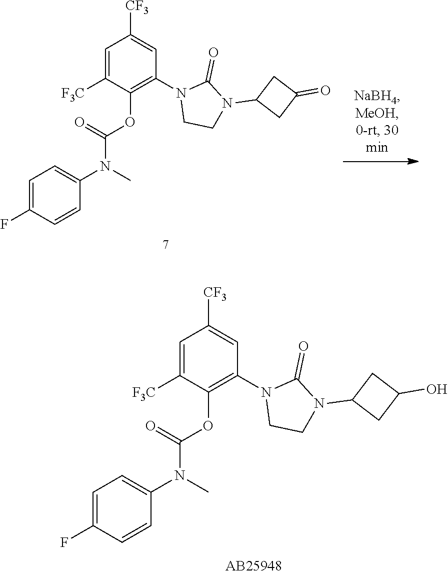 [SMILES: CN(C(=O)Oc1c(N2CCN(C3CC(=O)C3)C2=O)cc(C(F)(F)F)cc1C(F)(F)F)c1ccc(F)cc1 ; CN(C(=O)Oc1c(N2CCN(C3CC(O)C3)C2=O)cc(C(F)(F)F)cc1C(F)(F)F)c1ccc(F)cc1]

To a solution of compound 7 (95 mg, 0.17 mmol, 1.0 eq) in methanol (3 mL) at 0 °C was added NaBH₄ (17 mg, 0.34 mmol, 2.0 eq). The reaction mixture was stirred at rt for 30 min. After completion of the reaction monitored by TLC (Rf 0.3, PE:EA=1:1), NH₄C₁ (saturated aqueous, 2 mL) was added. The mixture was extracted with ethyl acetate (30 mL×2). The combined organic layer was washed with brine, dried over sodium sulfate and filtered. The filtrate was concentrated under reduced pressure to give a residue, which was purified by column chromatography (100-200 silica gel, 80% EA in PE as eluent) to afford title compound (AB25948; 58 mg, 61%) as pale white solid.

¹H NMR (400 MHz, CDCl₃): δ 7.70-7.85 (t, 2H), 7.25 (s, 2H), 7.05-7.09 (m, 2H), 4.04-4.12 (m, 2H), 3.73-3.82 (m, 2H), 3.54 (t, 2H), 3.47 (s, 1H) 3.33 (s, 2H), 2.61-2.65 (m, 2H), 2.14-2.19 (m, 2H); LC-MS: 536.15 [M+H]⁺.

### Example 35

### 2-(3-(1,1-dioxidotetrahydrothiophen-3-yl)-2-oxoimidazolidin-1-yl)-4,6-bis(trifluoromethyl)phenyl (4-fluorophenyl)(methyl)carbamate

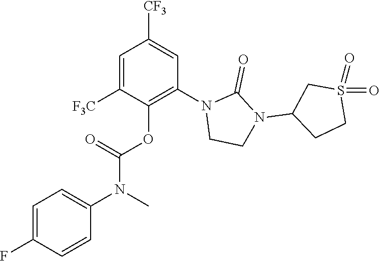 [SMILES: CN(C(=O)Oc1c(N2CCN(C3CCS(=O)(=O)C3)C2=O)cc(C(F)(F)F)cc1C(F)(F)F)c1ccc(F)cc1]

### Step 1: Synthesis of 2-(2,4-bis(trifluoromethyl)phenyl)-4,4,5,5-tetramethyl-1,3,2-dioxaborolane

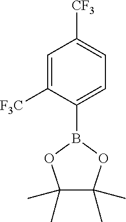 [SMILES: CC1(C)OB(c2ccc(C(F)(F)F)cc2C(F)(F)F)OC1(C)C]

A solution of 1-bromo-2,4-bis(trifluoromethyl)benzene (3 g, 0.01 mol) in 1,4 dioxane (90 mL) was purged with N2 in a sealed tube for 30 min and then added bispinacolato diboron (5.07 g, 0.02 mol), potassium acetate (1.96 g, 0.02 mol) and Pd(dppf)Cl₂·DCM (0.816 g, 0.001 mol) with stirring under inert atmosphere. The tightly closed sealed tube was heated to 100 °C in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4×75 mL); washings collected was concentrated under reduced pressure to afford the yellow sticky solid (crude 6 g, Quantitative). The product was taken as such to next step without further purification.

### Step 2: Synthesis of 2,4-bis(trifluoromethyl)phenol

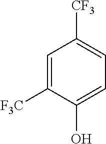 [SMILES: Oc1ccc(C(F)(F)F)cc1C(F)(F)F]

To a cold (0 °C) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0 °C and quenched with aq. sodium metabisulfite solution followed by extraction with EtOAc (2×100 mL). The organic layer collected dried over anhydrous Na₂SO₄, concentrated under reduced pressure to afford the crude. The crude was purified by column chromatography (100-200 silica gel, 10-15% EtOAc in Hexane as eluent) to afford the title compound as pale yellow liquid (1.5 g, 65% yield).

### Step 3: Synthesis of 2-iodo-4,6-bis(trifluoromethyl)phenol

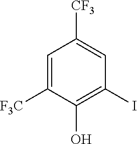 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20:6 mL) was cooled 0 °C in an ice bath with stirring. After 15 minutes, was added 12 (1.43 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 30% EtOAc in Hexane followed by 30% DCM in Hexane), the reaction mixture was cooled to 0 °C and then quenched with aq. sodium metabisulfite solution followed by extraction with EtOAc (2×100 mL). The organic layer collected dried over anhydrous Na₂SO₄, filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 4: Synthesis of (4-fluorophenyl)(methyl)carbamic chloride

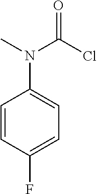

To a cold (0 °C) solution of triphosgene (1.6 g, 0.0128 mol) in DCM (40 mL) added a solution of N-methyl-4-fluoro aniline (1.89 g, 0.0064 mol) and pyridine (2.52 g, 2.6 mL, 0.032 mol) dropwise for over a period of 10 min. After that continued stirring at RT for 16 h. Progress of the reaction was monitored by TLC (R_{f} −0.7, 10% EtOAc in Hexane (×4)). After completion of the reaction, quenched the reaction mixture with 1M aq·HCl (50 mL) and then extracted with DCM (2×50 mL). The DCM layer separated was dried over anhydrous Na₂SO₄, filtered, and concentrated under reduced pressure to afford the title compound as green solid (1.8 g, 75%).

### Step 5: Synthesis of 2-iodo-4,6-bis(trifluoromethyl)phenyl (4-fluorophenyl)(methyl)carbamate

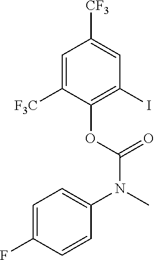 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80 °C for 4 hours. After completion of the reaction was confirmed by TLC (R_{f} −0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off-white solid (0.66 g, 93%).

### Step 6: Synthesis of 2-(2-oxoimidazolidin-1-yl)-4,6-bis(trifluoromethyl)phenyl (4-fluorophenyl)(methyl) carbamate

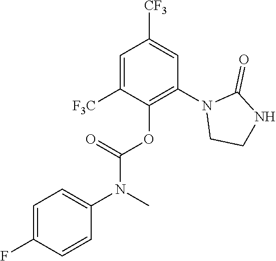 [SMILES: CN(C(=O)Oc1c(N2CCNC2=O)cc(C(F)(F)F)cc1C(F)(F)F)c1ccc(F)cc1]

2-iodo-4,6-bis(trifluoromethyl)phenyl (4-fluorophenyl)(methyl)carbamate (0.2 g, 0.00039 mol), 2-imidazilidinone (0.064 g, 0.0007 mol), copper (I)-iodide (0.037 g, 0.00019 mol), N,N′-dimethylethylenediamine (0.07 mL, 0.00039 mol), caesium fluoride (0.12 g, 0.00079 mol) and potassium carbonate (0.1 g, 0.00072 mol) were suspended in 1,4-dioxane priorly purged with N2 for 30 minutes (16 mL). The reaction mixture was heated at 90 °C in an oil-bath for 24 h. After completion of the reaction by TLC (R_{f} 0.4, 5% MeOH in DCM) the reaction mixture was concentrated under reduced pressure. The residue obtained was purified by column chromatography (100-200 silica gel, 0-3% MeOH in DCM as eluent) to afford the title compound as off-white solid (50 mg). The solid was re-purified by preparative TLC (solid phase: merck, 20×20 cm, silicagel 60 GF₂₅₄, 1 mm, PLC glass plate, 2% MeOH in DCM as eluent) to afford white solid (28 mg, 15%).

### Step 7: Synthesis of 2-(3-(1,1-dioxidotetrahydrothiophen-3-yl)-2-oxoimidazolidin-1-yl)-4,6-bis(trifluoromethyl)phenyl (4-fluorophenyl)(methyl)carbamate

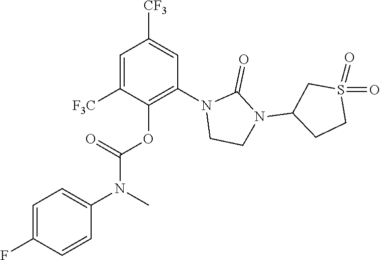 [SMILES: CN(C(=O)Oc1c(N2CCN(C3CCS(=O)(=O)C3)C2=O)cc(C(F)(F)F)cc1C(F)(F)F)c1ccc(F)cc1]

To a stirred solution of 2-(2-oxoimidazolidin-1-yl)-4,6-bis(trifluoromethyl)phenyl (4-fluorophenyl)(methyl)carbamate (0.3 g, 0.00064 mol) in DMF (15 mL) was purged with N2 for 5 minutes. To the above solution added K₃PO₄ (0.82 g, 0.00386 mol), KI (0.32 g, 0.0019 mmol), 3-bromotetrahydrothiophene 1,1-dioxide (0.39 g, 0.00196 mol) and at RT and continued purging for another 5 min. Heated the tightly closed sealed tube at 70 °C for 24 h. After completion of the reaction confirmed by TLC (R_{f} 0.4, 50% EtOAc in Hexane) the reaction mixture was diluted with cold water (150 mL) followed by extraction with ethyl acetate (2*100 mL). The organic fractions separated were combined, dried over anhydrous Na₂SO₄, filtered and concentrated under reduced pressure to afford crude. The crude residue was purified by silica gel chromatography (100-200 silica gel, 40-50% EtOAc in Hexane as eluent) to afford the title compound as impure. The impure compound was re-purified by preparative TLC (solid phase: merck, 20×20 cm, silicagel 60 GF₂₅₄, 1 mm, PLC glass plate, 60% EtOAc in Hexane as eluent) to afford the pure title compound as white solid (32 mg, 9%).

¹H NMR (400 MHz, Trifluoroacetic acid-D) δ 7.87 (m, 2H), 7.23 (m, 2H), 7.05 (m, 2H), 4.93 (s, 1H), 4.01 (s, 1H), 3.75 (m, 4H), 3.40 (m, 6H), 2.58 (m, 2H); MS(ESI): m/z 584.35 (M+H)⁺.

### Example 36

### 2-(3-((3-hydroxyazetidin-3-yl)methyl)-2-oxoimidazolidin-1-yl)-4,6-bis(trifluoromethyl)phenyl (4-fluorophenyl)(methyl-d₃)carbamate

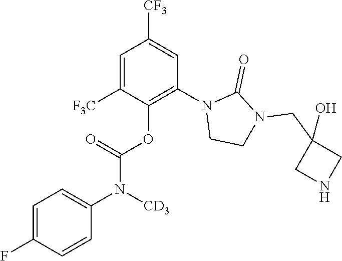 [SMILES: [2H]C([2H])([2H])N(C(=O)Oc1c(N2CCN(CC3(O)CNC3)C2=O)cc(C(F)(F)F)cc1C(F)(F)F)c1ccc(F)cc1]

### Step 1: Synthesis of 1-(2-hydroxy-3,5-bis(trifluoromethyl)phenyl)imidazolidine-2-one

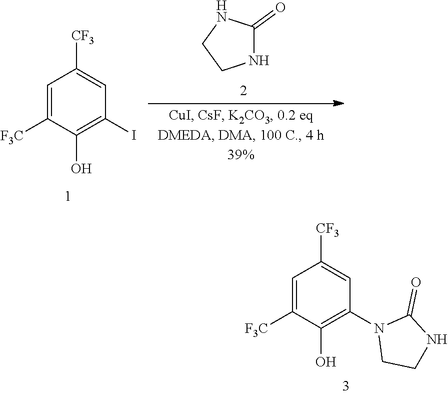 [SMILES: Oc1c(I)cc(C(F)(F)F)cc1C(F)(F)F ; O=C1NCCN1 ; O=C1NCCN1c1cc(C(F)(F)F)cc(C(F)(F)F)c1O]

To a solution of compound 1 (4.0 g, 11.26 mmol, 1.0 eq) and compound 2 (1.95 g, 22.5 mmol, 2.0 eq) in DMA (20 mL) was added CuI (1.07 g, 5.63 mmol, 0.5 eq), DMEDA (991 mg, 11.3 mmol, 1.0 eq), K₂CO₃ (3.13 g, 22.5 mmol, 2.0 eq) and CsF (3.42 g, 22.5 mmol, 2.0 eq) at rt. The reaction mixture was heated at 100 °C in an oil-bath for 4 h. After completion of the reaction monitored by TLC (R_{f} 0.5, PE:EA=2:1), water (20 mL) was added. The mixture was adjusted pH 6 with HCl (1 N). The mixture was extracted with ethyl acetate (EA, 100 mL×2). The combined organic layer was washed with brine, dried over Na₂SO₄ and filtered. The filtrate was concentrated under reduced pressure. The residue was purified by column chromatography (100-200 silica gel, 50% EA in PE as eluent) to afford compound 3 (1.39 g, 39%) as pale yellow solid.

### Step 2: Synthesis of tert-butyl (4-fluorophenyl)(methyl-d₃)carbamate

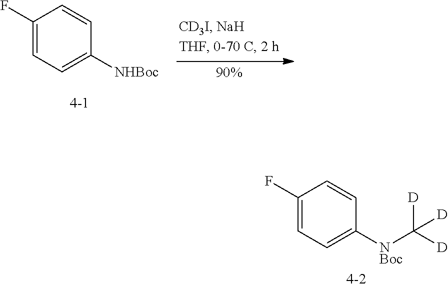 [SMILES: CC(C)(C)OC(=O)Nc1ccc(F)cc1 ; [2H]C([2H])([2H])N(C(=O)OC(C)(C)C)c1ccc(F)cc1]

To a solution of compound 4-1 (3.0 g, 14.2 mmol, 1.0 eq) in THF (10 mL) at 0 °C was added NaH (682 mg, 17.04 mmol, 1.2 eq). The reaction mixture was stirred at rt for 0.5 h. CD₃I (2.09 g, 28.4 mmol, 2.0 eq) was added at 0 °C. The reaction mixture was heated at 70 °C in an oil-bath for 2 h. After completion of the reaction monitored by TLC (R_{f} 0.4, PE:EA=10:1), the mixture was extracted with EA/H₂O (30 mL×2). The combined organic layer was washed with brine, dried over Na₂SO₄ and filtered. The filtrate was concentrated under reduced pressure. The residue was purified by column chromatography (100-200 silica gel, 15% EA in PE as eluent) to afford compound 4-2 (2.94 g, 90%) as a pale-yellow solid.

### Step 3: Synthesis of 4-fluoro-N-(methyl-d₃)aniline

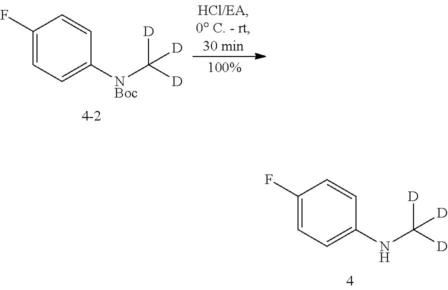 [SMILES: [2H]C([2H])([2H])N(C(=O)OC(C)(C)C)c1ccc(F)cc1 ; [2H]C([2H])([2H])Nc1ccc(F)cc1]

To a solution of HCl/EA (4 M, 10 mL) at 0 °C was added compound 4-2 (2.94 g, 12.8 mmol, 1.0 eq). The reaction was stirred at 0 °C for 0.5 h. The reaction was concentrated under reduced pressure to afford compound 4 (1.8 g, 100%) as off-white solid.

### Step 4: Synthesis of 2-(2-oxoimidazolidin-1-yl)-4,6-bis(trifluoromethyl)phenyl (4-fluorophenyl)(methyl-d₃)carbamate

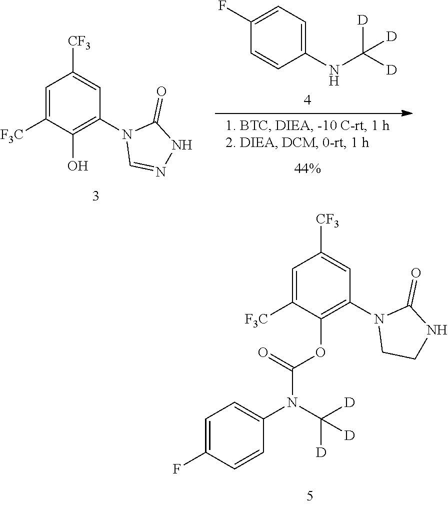 [SMILES: O=c1[nH]ncn1-c1cc(C(F)(F)F)cc(C(F)(F)F)c1O ; [2H]C([2H])([2H])Nc1ccc(F)cc1 ; [2H]C([2H])([2H])N(C(=O)Oc1c(N2CCNC2=O)cc(C(F)(F)F)cc1C(F)(F)F)c1ccc(F)cc1]

To a solution of compound 3 (300 mg, 0.95 mmol, 1.0 eq) in DCM (10 mL) at −10 °C was added DIEA (148 mg, 1.14 mmol, 1.5 eq) and a solution of triphosgene (127 mg, 0.42 mmol, 0.45 eq) in DCM (2 mL). The reaction mixture was stirred at it under N2 for 1 h. The reaction solution was concentrated under reduced pressure to remove the solvent. Then DCM (3 mL) added to above residue. The solution was used for next step.

To a solution compound 4 (380 mg, 1.43 mmol, 1.5 eq) in DCM (5 mL) at 0 °C was added the above solution. The mixture was stirred at rt under N2 for 1 h. After completion of the reaction monitored by TLC (R_{f} 0.3, PE:EA=2:1), HCl (1 N, 15 mL) was added. Then the mixture was extracted with DCM (20 mL×2). The combined organic layer was washed with brine, dried over Na₂SO₄, filtered, and concentrated under reduced pressure. The residue was purified by column chromatography (100-200 silica gel, 72% EA in PE as eluent) to afford compound 5 (200 mg, 44%) as off-white solid.

### Step 5: Synthesis of tert-butyl 3-((3-(2-(((4-fluorophenyl)(methyl-d₃)carbamoyl)oxy)-3,5-bis(trifluoromethyl)phenyl)-2-oxoimidazolin-1-yl)methyl)-3-hydroxyazetidine-1-carboxylate

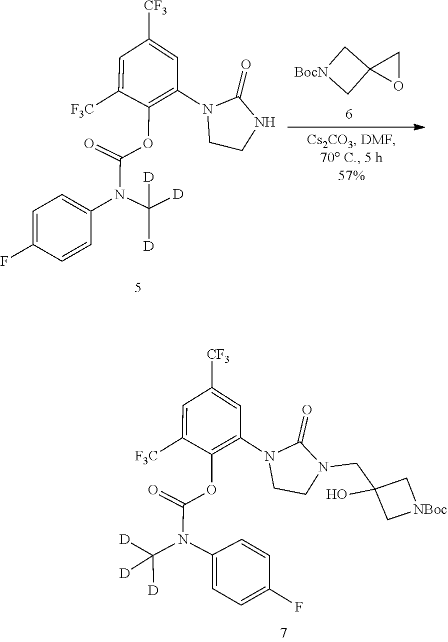 [SMILES: [2H]C([2H])([2H])N(C(=O)Oc1c(N2CCNC2=O)cc(C(F)(F)F)cc1C(F)(F)F)c1ccc(F)cc1 ; CC(C)(C)OC(=O)N1CC2(CO2)C1 ; [2H]C([2H])([2H])N(C(=O)Oc1c(N2CCN(CC3(O)CN(C(=O)OC(C)(C)C)C3)C2=O)cc(C(F)(F)F)cc1C(F)(F)F)c1ccc(F)cc1]

To a solution of compound 5 (200 mg, 0.43 mmol, 1.0 eq) and compound 6 (159 mg, 0.84 mmol, 2.0 eq) in DMF (3 mL) at rt was added Cs₂CO₃ (418 mg, 1.28 mmol, 3.0 eq). The reaction mixture was heated at 70 °C in an oil-bath for 5 h. After completion of the reaction monitored by TLC (Rf 0.3, PE:EA=2:1), the mixture was extracted with EA (30 mL×2). The combined organic layer was washed with brine, dried over Na₂SO₄ and filtered. The filtrate was concentrated under reduced pressure. The residue was purified by column chromatography (100-200 silica gel, 80% EA in PE as eluent) to afford compound 7 (160 mg, 57%) as white solid.

### Step 6: Synthesis of 2-(3-((3-hydroxyazetidin-3-yl)methyl)-2-oxoimidazolidin-1-yl)-4,6-bis(trifluoromethyl)phenyl (4-fluorophenyl)(methyl-d₃)carbamate

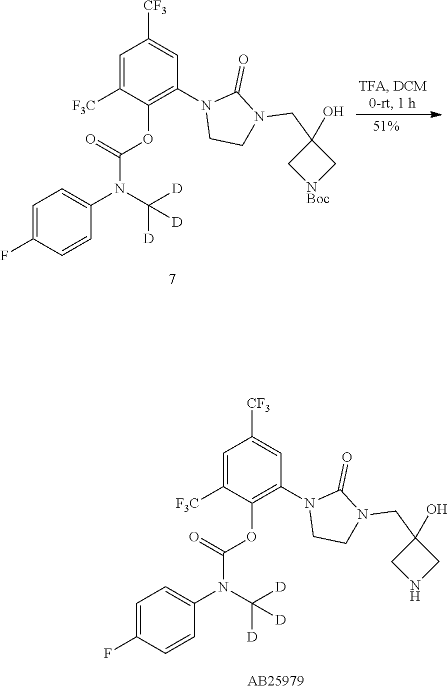 [SMILES: [2H]C([2H])([2H])N(C(=O)Oc1c(N2CCN(CC3(O)CN(C(=O)OC(C)(C)C)C3)C2=O)cc(C(F)(F)F)cc1C(F)(F)F)c1ccc(F)cc1 ; [2H]C([2H])([2H])N(C(=O)Oc1c(N2CCN(CC3(O)CNC3)C2=O)cc(C(F)(F)F)cc1C(F)(F)F)c1ccc(F)cc1]

A solution of compound 7 (160 mg, 0.24 mmol, 1.0 eq) in DCM/TFA (2 ml/2 mL) was stirred at rt for 1 h. After completion of the reaction checked by TLC (R⁸ 0.1, DCM:MeOH=10:1). The mixture was concentrated under reduced pressure. The residue was purified by reverse column (38% ACN in H₂O) and freeze-drying to give the title compound (AB25979; 80.8 mg, 59%) as white solid.

¹H NMR (400 MHz, CDCl₃): δ 9.47 (br, 1H), 8.88 (br, 1H), 7.77-7.83 (m, 2H,), 7.25-7.27 (m, 1H), 7.05-7.12 (m, 2H), 3.94 (m, 4H), 3.94 (m, 1H), 3.48-3.63 (m, 6H); LC-MS: 554.10 [M+H]⁺.

### Example 37

### 2-(3-((3-hydroxyazetidin-3-yl)methyl)-2-oxoimidazolidin-1-yl)-4,6-bis(trifluoromethyl)phenyl (3-chloro-2,4-difluorophenyl)(methyl-d₃)carbamate

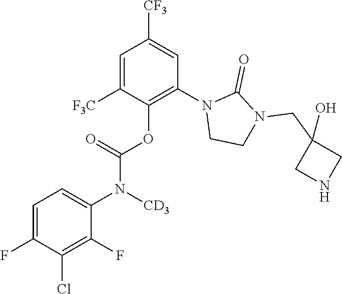 [SMILES: [2H]C([2H])([2H])N(C(=O)Oc1c(N2CCN(CC3(O)CNC3)C2=O)cc(C(F)(F)F)cc1C(F)(F)F)c1ccc(F)c(Cl)c1F]

### Step 1: Synthesis of N-(3-chloro-2,4-difluorophenyl)-2,2,2-trifluoroacetamide

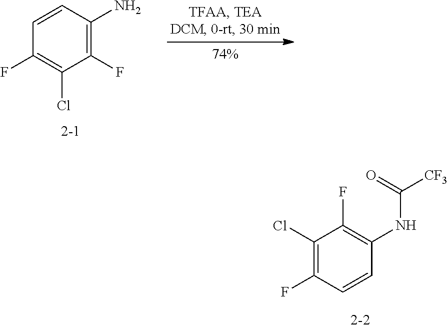 [SMILES: Nc1ccc(F)c(Cl)c1F ; O=C(Nc1ccc(F)c(Cl)c1F)C(F)(F)F]

To a solution of compound 2-1 (2.0 g, 12.2 mmol, 1.0 eq) in DCM (10 mL) at 0 °C was added TEA (1.6 g, 15.8 mmol, 1.3 eq), TFAA (3.08 g, 14.6 mmol, 1.2 eq). The reaction mixture was stirred at rt for 0.5 h. After completion of the reaction checked by TLC (R_{f} 0.6, PE:EA=10:1), the mixture was extracted with ethyl acetate (30 mL×2). The combined organic layer was washed with water and brine, dried over Na₂SO₄ and filtered. The filtrate was concentrated under reduced pressure. The residue was purified by column chromatography (100-200 silica gel, 8% EA in PE as eluent) to afford compound 2-2 (2.4 g, 74%) as white solid.

### Step 2: Synthesis of N-(3-chloro-2,4-difluorophenyl)-2,2,2-trifluoro-N(methyl-d₃)acetamide

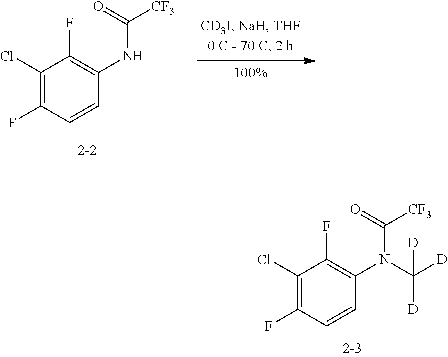 [SMILES: O=C(Nc1ccc(F)c(Cl)c1F)C(F)(F)F ; [2H]C([2H])([2H])N(C(=O)C(F)(F)F)c1ccc(F)c(Cl)c1F]

To a solution of compound 2-2 (2.4 g, 9.24 mmol, 1.0 eq) in THF (10 mL) at 0 °C was added NaH (443 mg, 11.08 mmol, 1.2 eq). The reaction mixture was stirred at it for 0.5 h. CD₃I (2.72 g, 18.49 mmol, 2.0 eq) was added at 0 °C. The reaction mixture was heated at 70 °C in an oil-bath for 2 h. After completion of the reaction monitored by TLC (R_{f} 0.5, PE:EA=10:1), the mixture was quenched with saturated ammonium chloride (50 mL), extracted with EA (50 mL×2). The combined organic layer was washed with brine, dried over Na₂SO₄, filtered, and concentrated under reduced pressure to afford the crude compound 2-3 (2.6 g, 100%) as yellow oil, which was used for the next reaction without further purification.

### Step 3: Synthesis of 4-fluoro-N-(methyl-d₃)aniline

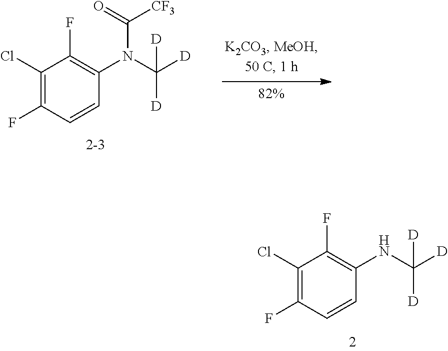 [SMILES: [2H]C([2H])([2H])N(C(=O)C(F)(F)F)c1ccc(F)c(Cl)c1F ; [2H]C([2H])([2H])Nc1ccc(F)c(Cl)c1F]

To a solution of compound 2-3 (2.6 g, 9.42 mmol, 1.0 eq) in methanol (20 mL) at rt was added K₂CO₃ (1.96 g, 14.13 mmol, 1.5 eq). The reaction mixture was heated at 50 °C for 1 h. The mixture was concentrated, diluted with water (50 mL) and extracted with ethyl acetate (40 mL×2). The combined organic layer was washed with brine, dried over Na₂SO₄ and filtered. The filtrate was concentrated under reduced pressure. The residue was purified by column chromatography (100-200 silica gel, 10% EA in PE as eluent) to afford compound 2 (1.4 g, 82%) as yellow oil.

### Step 4: Synthesis of 2-(2-oxoimidazolidin-1-yl)-4,6-bis(trifluoromethyl)phenyl (3-chloro-2,4-difluorophenyl)(methyl-d₃)carbamate

To a solution of compound 2 (500 mg, 1.59 mmol, 1.0 eq) in DCM (10 mL) at −10 °C were added DIEA (308 mg, 2.38 mmol, 1.5 eq) and a solution of triphosgene (212 mg, 0.72 mmol, 0.45 eq) in DCM (2 mL). The reaction mixture was stirred at it under N2 for 1 h. The reaction solution was concentrated under reduced pressure to remove the solvent. The residue was dissolved in DCM (3 mL). The solution was used for next step.

To a solution compound 2 (430 mg, 2.38 mmol, 1.5 eq) in DCM (5 mL) at 0 °C was added the chloroformate solution prepared above. The mixture was stirred at rt under N2 for 1 h. After completion of the reaction monitored by TLC (R_{f} 0.3, PE:EA=2:1), HCl (1 N, 15 mL) was added to quench the reaction. Then the mixture was extracted with DCM (30 mL×2). The combined organic layer was washed with brine, dried over Na₂SO₄ and filtered. The solution was concentrated under reduced pressure. The residue was purified by column chromatography (100-200 silica gel, 70% EA in PE as eluent) to afford compound 3 (80 mg, 9.6%) as off-white solid.

### Step 5: Synthesis of tert-butyl 3-((-3-(2-(((3-chloro-2,4-difluorophenyl)(methyl-d₃)carbamoyl)oxy)-3,5-bis(trifluoromethyl)phenyl)-2-oxoimidazolidin-1-yl)methyl)-3-hydroxyazetidine-1-carboxylate

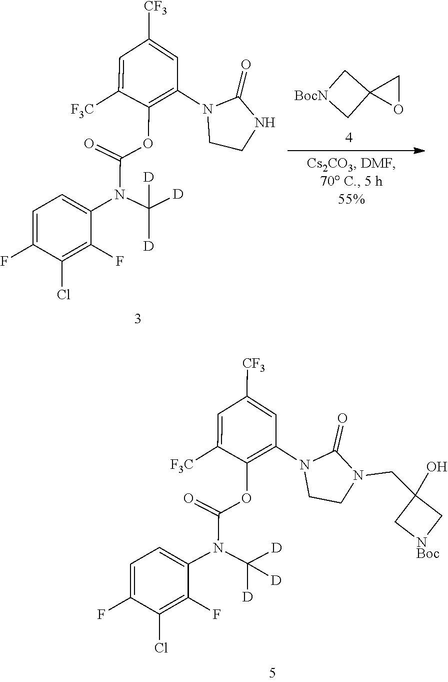 [SMILES: [2H]C([2H])([2H])N(C(=O)Oc1c(N2CCNC2=O)cc(C(F)(F)F)cc1C(F)(F)F)c1ccc(F)c(Cl)c1F ; CC(C)(C)OC(=O)N1CC2(CO2)C1 ; [2H]C([2H])([2H])N(C(=O)Oc1c(N2CCN(CC3(O)CN(C(=O)OC(C)(C)C)C3)C2=O)cc(C(F)(F)F)cc1C(F)(F)F)c1ccc(F)c(Cl)c1F]

To a solution of compound 3 (80 mg, 0.154 mmol, 1.0 eq) and compound 4 (57 mg, 0.307 mmol, 2.0 eq) in DMF (3 mL) at rt was added Cs₂CO₃ (150 mg, 0.46 mmol, 3.0 eq). The reaction mixture was heated at 70 °C in an oil-bath for 5 h. After completion of the reaction checked by LC-MS and TLC (R_{f} 0.3, PE:EA=2:1), the mixture was extracted with EA (30 mL×2). The combined organic layer was washed with brine, dried over Na₂SO₄ and filtered. The solution was concentrated under reduced pressure. The residue was purified by column chromatography (100-200 silica gel, 80% EA in PE as eluent) to afford compound 5 (60 mg, 55%) as white solid.

### Step 6: Synthesis of 2-(3-((3-hydroxyazetidin-3-yl)methyl)-2-oxoimidazolidin-1-yl)-4,6-bis(trifluoromethyl)phenyl (3-chloro-2,4-difluorophenyl)(methyl-d₃)carbamate

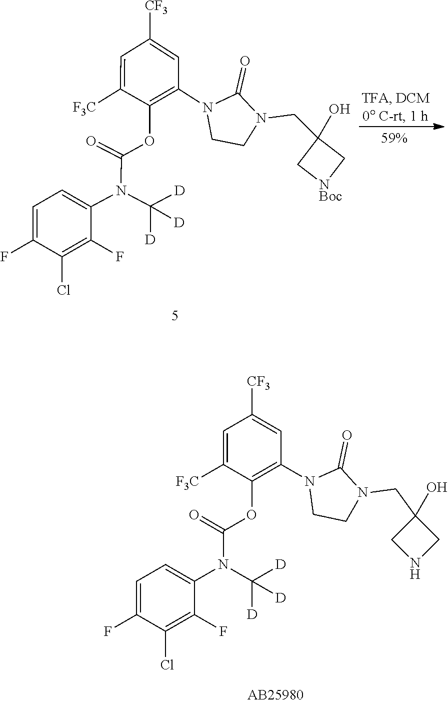 [SMILES: [2H]C([2H])([2H])N(C(=O)Oc1c(N2CCN(CC3(O)CN(C(=O)OC(C)(C)C)C3)C2=O)cc(C(F)(F)F)cc1C(F)(F)F)c1ccc(F)c(Cl)c1F ; [2H]C([2H])([2H])N(C(=O)Oc1c(N2CCN(CC3(O)CNC3)C2=O)cc(C(F)(F)F)cc1C(F)(F)F)c1ccc(F)c(Cl)c1F]

To a solution of compound 5 (60 mg, 0.085 mmol, 1.0 eq) in DCM (2 mL) at 0 °C was added TFA (2 mL). The reaction mixture was stirred at rt for 1 h. The mixture was concentrated under reduced pressure. The residue was purified by C18 reverse column (40% ACN in H₂O) and freeze-dried to give title compound (AB25980; 30.8 mg, 59%) as white solid.

¹H NMR (400 MHz, CDCl₃): δ 9.61-9.69 (m, 1H), 8.84-9.01 (m, 1H), 7.80-7.84 (t, 2H), 7.19-7.31 (m, 1H), 3.96-3.97 (s, 4H), 3.83 (s, 1H), 3.54-3.70 (t, 6H); LC-MS: 606.05[M+H]⁺.

### Example 38

### 2,4-bis(trifluoromethyl)-6-(3-((3-hydroxyazetidin-3-yl)methyl)-2-oxoimidazolidin-1-yl)phenyl (3-chloro-2,4-difluorophenyl)(methyl)carbamate

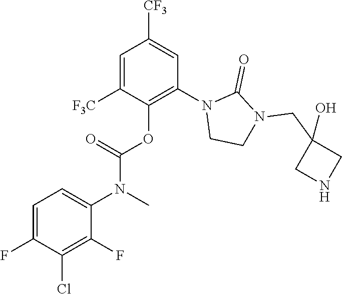 [SMILES: CN(C(=O)Oc1c(N2CCN(CC3(O)CNC3)C2=O)cc(C(F)(F)F)cc1C(F)(F)F)c1ccc(F)c(Cl)c1F]

### Step 1: Synthesis of tert-butyl 3-((3-(2-(((3-chloro-2,4-difluorophenyl)(methyl)carbamoyl)oxy)-3,5-bis(trifluoromethyl)phenyl)-2-oxoimidazolidin-1-yl)methyl)-3-hydroxyazetidine-1-carboxylate

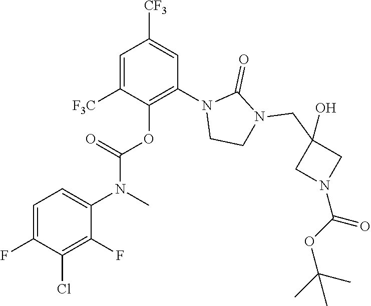 [SMILES: CN(C(=O)Oc1c(N2CCN(CC3(O)CN(C(=O)OC(C)(C)C)C3)C2=O)cc(C(F)(F)F)cc1C(F)(F)F)c1ccc(F)c(Cl)c1F]

To a solution of 2-(2-oxoimidazolidin-1-yl)-4,6-bis(trifluoromethyl)phenyl N-(3-chloro-2,4-difluorophenyl)-N-methylcarbamate (0.085 g, 0.16 mmol) 5 mL anhydrous dimethylformamide was added azetidine-N═Boc-epoxide (0.79 g, 3.44 mmol) and cesium carbonate (0.14 g, 0.429 mmol) were added under inert atmosphere and stirred at 70 °C for 72 h. The reaction was diluted with water (100 mL). The aqueous phase was separated and extracted with EtOAc twice. The combined organic extracts were washed with brine, dried over anhydrous sodium sulfate and concentrated. The crude product was purified by column chromatography on silica gel using a gradient solvent system of 0 to 80% of ethyl acetate in hexanes to afford the titled compound as an off white solid (28 mg, 24%) ESIMS: m/z 703.73 [(M+H)⁺].

### Step 2: Synthesis of 2-(3-((-hydroxyazetidin-3-yl)methyl)-2-oxoimidazolidin-1-yl)-4,6-bis(trifluoromethyl)phenyl (3-chloro-2,4-difluorophenyl)(methyl)carbamate

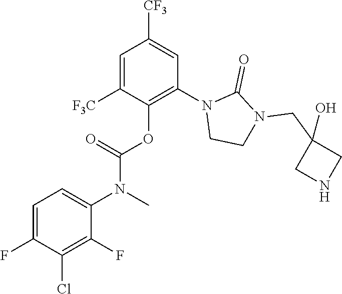 [SMILES: CN(C(=O)Oc1c(N2CCN(CC3(O)CNC3)C2=O)cc(C(F)(F)F)cc1C(F)(F)F)c1ccc(F)c(Cl)c1F]

To a solution of tert-butyl 3-((3-(2-(((3-chloro-2,4-difluorophenyl)(methyl)carbamoyl)oxy)-3,5-bis(trifluoromethyl)phenyl)-2-oxoimidazolidin-1-yl)methyl)-3-hydroxyazetidine-1-carboxylate (0.025 g, 0.035 mmol), in 9 mL dichloromethane cooled to 0 °C. 1 mL trifluoro acetic acid was added and stirred at room temperature for 3 h. The solvent was concentrated to an oil under reduced pressure. The oil was triturated with ethylether to give an off white sold. The solid was filtered and dried under high vacuum to obtain the pure title compound as off white solid (0.010 g, 47%).

¹H NMR (400 MHz, CDCl₃) δ 7.80 (m, 2H), 7.25 (m, 2H), 4.2 (m, 2H), 4.06 (m, 2H), 3.78 (m, 2H), 3.98 (m, 2H), 3.73, (m, 3H) 3.61 (m, 2H); ESIMS: m/z 603.673 [(M+H)⁺].

### Example 39

### tert-butyl 3-((3-(2-(((3-chloro-2,4-difluorophenyl)(methyl-d₃)carbamoyl)oxy)-3,5-bis(trifluoromethyl)phenyl)-2-oxoimidazolidin-1-yl)methyl)-3-(sulfamoyloxy)azetidine-1-carboxylate

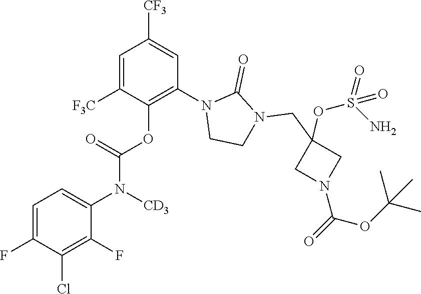 [SMILES: [2H]C([2H])([2H])N(C(=O)Oc1c(N2CCN(CC3(OS(N)(=O)=O)CN(C(=O)OC(C)(C)C)C3)C2=O)cc(C(F)(F)F)cc1C(F)(F)F)c1ccc(F)c(Cl)c1F]

### Step 1: Synthesis of N-(3-chloro-2,4-difluorophenyl)-2,2,2-trifluoroacetamide

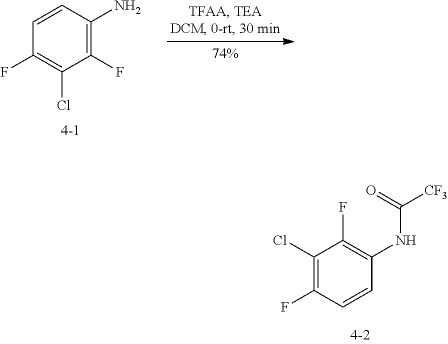 [SMILES: Nc1ccc(F)c(Cl)c1F ; O=C(Nc1ccc(F)c(Cl)c1F)C(F)(F)F]

To a solution of compound 4-1 (2.0 g, 12.2 mmol, 1.0 eq) in DCM (3 mL) was added TEA (1.6 g, 15.8 mmol, 1.3 eq), TFAA (3.08 g, 14.6 mmol, 1.2 eq) at 0 C. The reaction mixture was stirred for 0.5 h at rt. After completion of the reaction by TLC (R_{f} 0.6, PE:EA=10:1), The mixture was extracted with EA (30 mL×2). The combined organic layer was washed with brine, dried with Na₂SO₄ and filtered. The filtrate was concentrated under reduced pressure. The residue was purified by column chromatography (100-200 silica gel, 8% EA in PE as eluent) to afford compound 4-2 (2.4 g, 74%) as white solid.

### Step 2: Synthesis of N-(3-chloro-2,4-difluorophenyl)-2,2,2-trifluoro-N-(methyl-d₃)acetamide

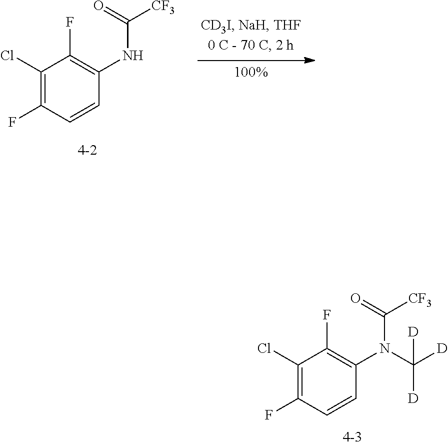 [SMILES: O=C(Nc1ccc(F)c(Cl)c1F)C(F)(F)F ; [2H]C([2H])([2H])N(C(=O)C(F)(F)F)c1ccc(F)c(Cl)c1F]

To a solution of compound 4-2 (2.4 g, 9.24 mmol, 1.0 eq) in THF (10 mL), was added NaH (443 mg, 11.08 mmol, 1.2 eq) at 0 °C. The reaction mixture was stirred for 0.5 h at rt. CD₃I (2.72 g, 18.49 mmol, 2.0 eq) was added at 0 °C. The reaction mixture was heated at 70 °C in an oil-bath for 2 h. After completion of the reaction checked with TLC (R_{f} 0.5, PE:EA=10:1). The mixture was diluted with aqueous ammonium chloride (40 mL) and extracted with ethyl acetate (30 mL×2). The combined organic layer was washed with brine, dried with Na₂SO₄ and filtered. The filtrate was concentrated under reduced pressure to afford crude compound 4-3 as yellow oil (2.6 g, 100%).

### Step 3: Synthesis of 3-chloro-2,4-difluoro-N-(methyl-d₃)aniline

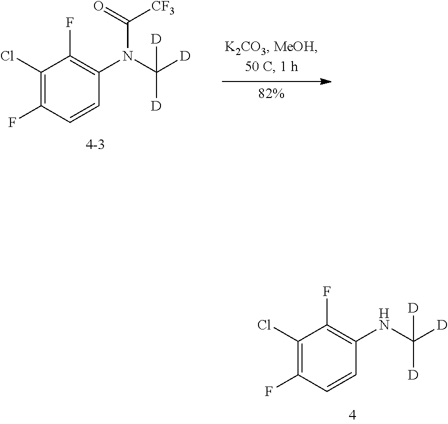 [SMILES: [2H]C([2H])([2H])N(C(=O)C(F)(F)F)c1ccc(F)c(Cl)c1F ; [2H]C([2H])([2H])Nc1ccc(F)c(Cl)c1F]

To a solution of compound 4-3 (2.6 g, 9.42 mmol, 1.0 eq) in methanol (30 mL) was added K₂CO₃ (1.96 g, 14.13 mmol, 1.5 eq) at rt. The reaction mixture was heated at 50 °C for 1 h. The mixture was dilute with ethyl acetate (100 mL), washed with water and brine, dried with Na₂SO₄ and filtered. The filtrate was concentrated under reduced pressure. The residue was purified by column chromatography (100-200 silica gel, 10% EA in PE as eluent) to afford compound 4 (1.4 g, 82%) as yellow oil.

### Step 4: Synthesis of 1-(2-hydroxy-3,5-bis(trifluoromethyl)phenyl)imidazolidine-2-one

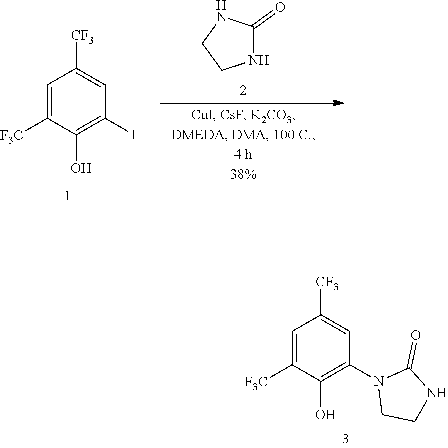 [SMILES: Oc1c(I)cc(C(F)(F)F)cc1C(F)(F)F ; O=C1NCCN1 ; O=C1NCCN1c1cc(C(F)(F)F)cc(C(F)(F)F)c1O]

To a solution of compound 1 (3.0 g, 8.45 mmol, 1.0 eq) and compound 2 (1.45 g, 16.9 mmol, 2.0 eq) in DMA (10 mL) were added CuI (803 mg, 4.22 mmol, 0.5 eq), DMEDA 744 mg, 8.45 mmol, 1.0 eq), K₂CO₃ (2.35 g, 16.9 mmol, 2.0 eq) and CsF (2.57 g, 16.9 mmol, 2.0 eq) at rt. The reaction mixture was heated at 100 °C in an oil-bath for 4 h. After completion of the reaction monitored with LC-MS and TLC (R_{f} 0.3, PE:EA=2:1), water (20 mL) was added. The mixture was adjusted to pH 6 with HCl (1 N). The mixture was extracted with ethyl acetate (50 mL×2). The combined organic layer was washed with brine, dried with Na₂SO₄ and filtered, and concentrated under reduced pressure. The residue was purified by column chromatography (100-200 silica gel, 50% EA in PE as eluent) to afford compound 3 (1.02 g, 38%) as pale yellow solid.

### Step 5: Synthesis of 2-(2-oxoimidazolidin-1-yl)-4,6-bis(trifluoromethyl)phenyl (3-chloro-2,4-difluorophenyl)(methyl-d₃)carbamate

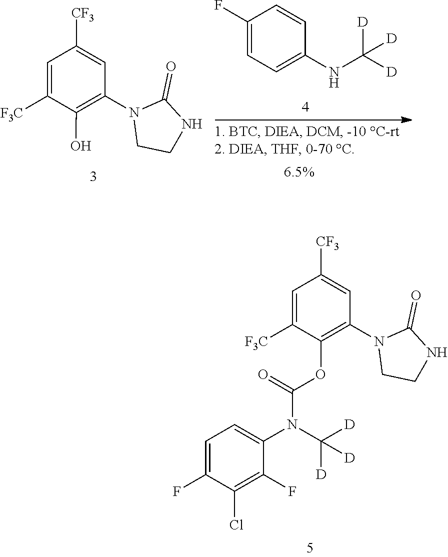 [SMILES: O=C1NCCN1c1cc(C(F)(F)F)cc(C(F)(F)F)c1O ; [2H]C([2H])([2H])Nc1ccc(F)cc1 ; [2H]C([2H])([2H])N(C(=O)Oc1c(N2CCNC2=O)cc(C(F)(F)F)cc1C(F)(F)F)c1ccc(F)c(Cl)c1F]

To a solution of compound 3 (1.02 g, 3.24 mmol, 1.0 eq) and DIEA (628 mg, 4.87 mmol, 1.5 eq) in DCM (20 mL) at −10 °C was added a solution of triphosgene (140 mg, 1.46 mmol, 0.45 eq) in DCM (2 mL). The reaction mixture was stirred for 1 h at rt under N2. The reaction solution was concentrated under reduced pressure to remove the solvent. THF (10 mL) added to the residue above. The solution was used for next step.

To a solution compound 4 (877 mg, 4.87 mmol, 1.5 eq) in THF (15 mL) at 0 °C was added the solution. The mixture was heated at 70 °C under N2 for 12 h. HCl (1 N, 15 mL) was added to quench the reaction. Then the mixture was extracted with ethyl acetate (30 mL×2). The combined organic layer was washed with brine, dried with Na₂SO₄ and filtered. The filtrate was concentrated under reduced pressure. The residue was purified by column chromatography (100-200 silica gel, 72% EA in PE as eluent) to afford compound 5 (110 mg, 6.5%) as off-white solid.

### Step 6: Synthesis of tert-butyl 3-((3-(2-(((3-chloro-2,4-difluorophenyl)(methyl-d₃)carbamoyl)oxy)-3,5-bis(trifluoromethyl)phenyl)-2-oxoimidazolidin-1-yl)methyl)-3-hydroxyazetidine-1-carboxylate

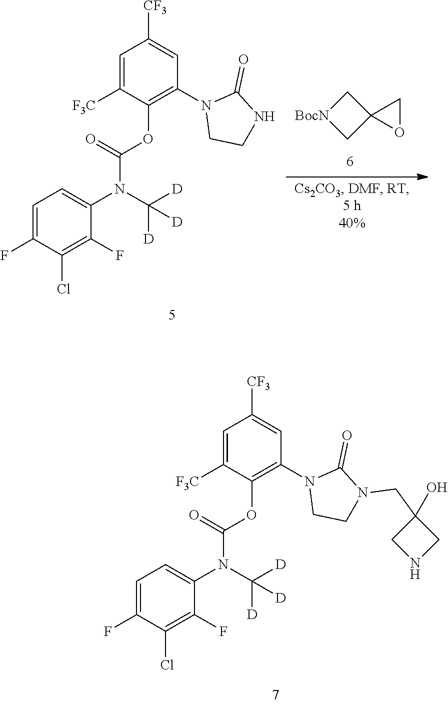 [SMILES: [2H]C([2H])([2H])N(C(=O)Oc1c(N2CCNC2=O)cc(C(F)(F)F)cc1C(F)(F)F)c1ccc(F)c(Cl)c1F ; CC(C)(C)OC(=O)N1CC2(CO2)C1 ; [2H]C([2H])([2H])N(C(=O)Oc1c(N2CCN(CC3(O)CNC3)C2=O)cc(C(F)(F)F)cc1C(F)(F)F)c1ccc(F)c(Cl)c1F]

To a solution of compound 5 (110 mg, 0.211 mmol, 1.0 eq) and compound 6 (78 mg, 0.423 mmol, 2.0 eq) in DMF (5 mL) was added Cs₂CO₃ (206 mg, 0.636 mmol, 3.0 eq) at rt. The reaction mixture was stirred at rt for 5 h. After completion of the reaction checked by TLC (R_{f} 0.3, PE:EA=2:1), the mixture was diluted with EA (100 mL), washed with brine, dried with Na₂SO₄ and filtered. The filtrate was concentrated under reduced pressure. The residue was purified by column chromatography (100-200 silica gel, 80% EA in PE as eluent) to afford the title compound 7 (60 mg, 40%) as white solid.

### Step 7: Synthesis of tert-butyl 3-((3-(2-(((3-chloro-2,4-difluorophenyl)(methyl-d₃)carbamoyl)oxy)-3,5-bis(trifluoromethyl)phenyl)-2-oxoimidazolidin-1-yl)methyl)-3-(sulfamoyloxy)azetidine-1-carboxylate

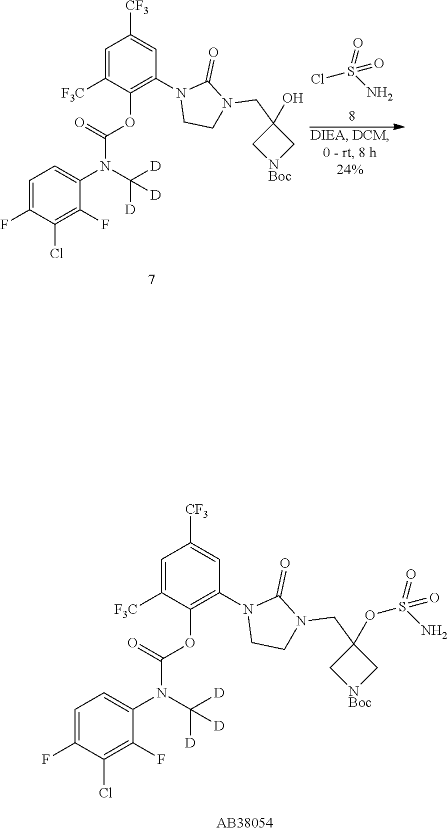

To a solution of compound 7 (60 mg, 0.085 mmol, 1.0 eq) in DCM (10 mL) at 0 °C was added DIEA (33 mg, 0.255 mmol, 3.0 eq), and compound 8 (29 mg, 0.255 mmol, 3.0 eq). The reaction was stirred at rt for 8 h. The mixture was diluted with EA (60 mL), washed with water, brine, dried with Na₂SO₄ and filtered, and concentrated under reduced pressure. The residue was purified by prep-HPLC to afford the title compound (AB38054; 16.1 mg, 24%) as off-white solid.

¹H NMR (400 MHz, CDCl₃): δ 7.79-7.85 (d, 2H,), 7.30-7.32 (m, 0.68H), 7.24 (m, 0.39H), 7.07-7.11 (m, 1H), 5.50-5.75 (d, 2H), 4.36 (s, 2H), 4.00-4.05 (t, 2H), 3.70 (m, 4H), 1.45 (s, 9H); LC-MS: 802.35 [M+H+H₂O]⁺.

### Example 40

### 2-(3-(3-(dimethylamino)-2-hydroxypropyl)-2-oxoimidazolidin-1-yl)-4,6-bis(trifluoromethyl)phenyl (3-chloro-2,4-difluorophenyl)(methyl-d₃)carbamate

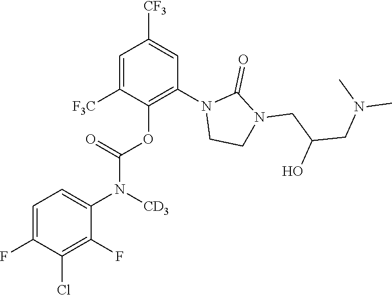 [SMILES: [2H]C([2H])([2H])N(C(=O)Oc1c(N2CCN(CC(O)CN(C)C)C2=O)cc(C(F)(F)F)cc1C(F)(F)F)c1ccc(F)c(Cl)c1F]

### Step 1: Synthesis of (3-chloro-2,4-difluorophenyl)(methyl-d₃)carbamic chloride

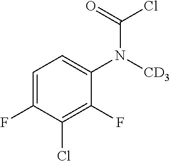 [SMILES: [2H]C([2H])([2H])N(C(=O)Cl)c1ccc(F)c(Cl)c1F]

To a solution of (3-chloro-2,4-difluoro)(methyl-d₃)aniline (0.58 g, 3.22 mmol) and pyridine (0.73 g, 9.34 mmol) in 12 mL of dichloromethane at 0 °C, triphosgene (0.69 g, 2.33 mmol) dissolved in 6 mL dichloromethane was added dropwise under inert atmosphere. The reaction was stirred at ambient temperature (room temperature) for 2 h. The reaction mixture was diluted with 20 mL dichloromethane and extracted with 20 mL 1N HCl. The organic layer was separated, dried over with anhydrous sodium sulfate, filtered and concentrated to a solid under reduced pressure.

### Step 2: Synthesis of 2-iodo-2,4-bis(trifluoromethyl)phenyl (3-chloro-2,4-difluorophenyl)(methyl-d₃)carbamate

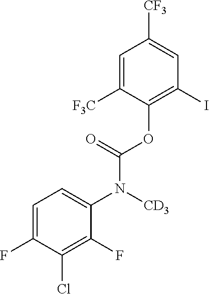 [SMILES: [2H]C([2H])([2H])N(C(=O)Oc1c(I)cc(C(F)(F)F)cc1C(F)(F)F)c1ccc(F)c(Cl)c1F]

2-iodo-4,6-bis(trifluoromethyl)phenol (570 mg, 1.60 mmol) and (3-chloro-2,4-difluorophenyl)(methyl-d₃)carbamic chloride (300 mg, 1.23 mmol) were dissolved into anhydrous pyridine (5 mL). This solution was stirred at 90 °C for 4 hours. The reaction was cooled to RT and concentrated down. The residual solid was partitioned between EtOAc and 1N aqueous HCL. The aqueous phase was separated and extracted with EtOAc twice. The combined organic extracts were washed with brine, dried over anhydrous sodium sulfate and concentrated. The crude product was purified by column chromatography on silica gel using a gradient solvent system of 0 to 20% of ethyl acetate in hexanes to afford the titled compound as a yellow oil (227 mg, 48%) ESIMS: m/z 603.43 [(M+H+41)⁺].

### Step 3: Synthesis of 2-(2-oxoimidazolidin-1-yl)-4,6-bis(trifluoromethyl)phenyl (3-chloro-2,4-difluorophenyl)(methyl-d₃)carbamate

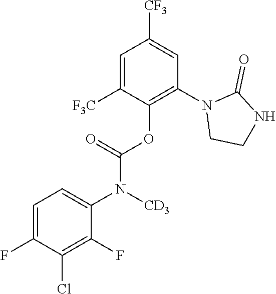 [SMILES: [2H]C([2H])([2H])N(C(=O)Oc1c(N2CCNC2=O)cc(C(F)(F)F)cc1C(F)(F)F)c1ccc(F)c(Cl)c1F]

2-iodo-2,4-bis(trifluoromethyl)phenyl (3-chloro-2,4-difluorophenyl)(methyl-d₃)carbamate (0.242 mmol, 126 mg), 2-imidazolidone (0.4548 mmol, 39 mg), copper (I) iodide (0.1157 mmol, 22 mg), cesium fluoride (0.4538 mmol, 69 mg), N,N′-dimethylethylenediamine (0.2269 mmol, 25 uL) and anhydrous powered potassium carbonate (0.4538 mmol, 63 mg) were added to degassed anhydrous 1,4-dioxane (5.0 mL) under nitrogen. The resulting suspension was stirred at 90 °C overnight. The reaction was cooled to room temperature, filtered and the filtrate was concentrated down to yield a semi-solid. The crude product was purified by column chromatography on silica gel using a gradient solvent system of 0 to 100% of ethyl acetate in hexanes to afford the titled compound as a yellow oil (6 mg, 5%). ESIMS: m/z 521.54 [(M+H)⁺].

### Step 4: Synthesis of 2-(3-(oxiran-2-ylmethyl)-2-oxoimidazolidin-1-yl)-4,6-bis(trifluoromethyl)phenyl (3-chloro-2,4-difluorophenyl)(methyl-d₃)carbamate

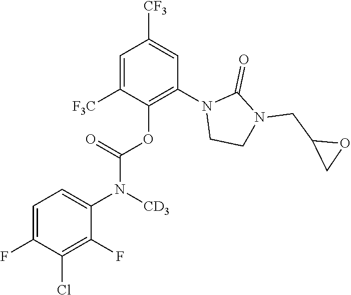 [SMILES: [2H]C([2H])([2H])N(C(=O)Oc1c(N2CCN(CC3CO3)C2=O)cc(C(F)(F)F)cc1C(F)(F)F)c1ccc(F)c(Cl)c1F]

To a solution of 2-(2-oxoimidazolidin-1-yl)-4,6-bis(trifluoromethyl)phenyl (3-chloro-2,4-difluorophenyl)(methyl-d₃)carbamate (0.165 g, 0.32 mmol) 12 mL anhydrous dimethylformamide was added and purged with nitrogen for 10 minutes. To the above reaction solution was added K₃PO₄ (0.45 g, 2.12 mmol) and purged with Nitrogen for 10 minutes. After stirring for 30 minutes at room temperature, the reaction mixture was cooled to 0 °C and epibromohydrin (2.12 mL, 2.12 mmol), potassium iodide (0.23 g, 1.41 mmol) were added. The reaction mixture was stirred at room temperature for 20 h. The reaction mixture was diluted with water (20 mL) and extracted with ethyl acetate (2×50 mL). The combined organic fractions were dried over anhydrous sodium sulfate, filtered and concentrated under reduced pressure to an oil. The crude oil was purified on 12 g silica gel cartridge using 0 to 100% ethyl acetate as eluent to afford title compound as an oil (0.025 g, 13% yield). ESIMS: m/z 577.30 [(M+H)⁺]

### Step 5: Synthesis of 2-(3-(3-(dimethylamino)-2-hydroxypropyl)-2-oxoimidazolidin-1-yl)-4,6-bis(trifluoromethyl)phenyl (3-chloro-2,4-difluorophenyl)(methyl-d₃)carbamate

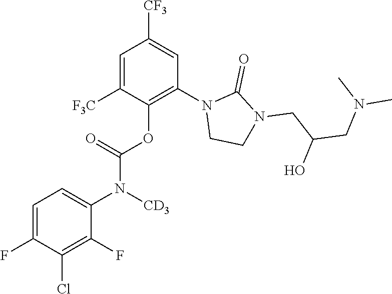 [SMILES: [2H]C([2H])([2H])N(C(=O)Oc1c(N2CCN(CC(O)CN(C)C)C2=O)cc(C(F)(F)F)cc1C(F)(F)F)c1ccc(F)c(Cl)c1F]

To a solution of 2-(3-(oxiran-2-ylmethyl)-2-oxoimidazolidin-1-yl)-4,6-bis(trifluoromethyl)phenyl (3-chloro-2,4-difluorophenyl)(methyl-d₃)carbamate (0.075 g, 1.29 mmol) in 9 mL anhydrous methanol at 0 °C. 2M dimethylamine (6 mL, 12 mmol) was added. The reaction was stirred for 2 h at room temperature. The solvent was removed under reduced pressure. The oil obtained was purified on 12 g silica gel column using 0-100% ethyl acetate in hexanes as eluent, to afford 30 mg of title compound as off white solid (37% yield).

¹H NMR (400 MHz, CDCl₃) δ 7.89 (s, 1H), 7.74 (d, 1H), 7.42 (m, 1H), 7.07 (m, 1H), 4.42 (m, 1H),), 3.86 (m, 1H), 3.74 (m, 2H), 3.46 (m, 2H), 3.25 (m, 2H), 3.26 (m, 2H), 2.89 (ddd, J=4 Hz, 6H); ESIMS: m/z 622.77 [(M+H)⁺].

### Example 41

### 2-((3S,4R)-3,4-dihydroxy-2-oxopyrrolidin-1-yl)-4,6-bis(trifluoromethyl)phenyl (4-fluorophenyl)(methyl-d₃)carbamate

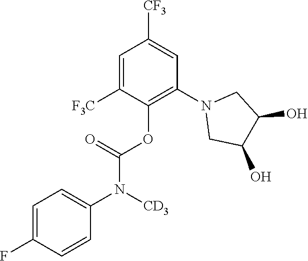 [SMILES: [2H]C([2H])([2H])N(C(=O)Oc1c(N2C[C@@H](O)[C@@H](O)C2)cc(C(F)(F)F)cc1C(F)(F)F)c1ccc(F)cc1]

### Step 1: Synthesis of 2-nitro-4,6-bis(trifluoromethyl)phenol

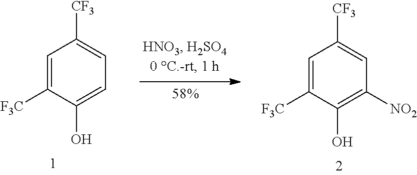 [SMILES: Oc1ccc(C(F)(F)F)cc1C(F)(F)F ; O=[N+]([O-])c1cc(C(F)(F)F)cc(C(F)(F)F)c1O]

To a solution of compound 1 (2.0 g, 8.7 mmol, 1.0 eq) in H₂SO₄ (20 mL) was added HNO₃ (927 mg, 9.57 mmol, 1.1 eq) at 0 °C. The reaction mixture was stirred at rt for 1 h. After completion of the reaction by TLC (R_{f} 2=0.1, PE/EA=1/1), the reaction solution was poured into water (50 mL). The mixture was extracted with EA (50 mL×2). The combined organic layer was washed with brine, dried over Na₂SO₄ and filtered. The filtration was concentrated under reduced pressure to afford compound 2 (1.4 g, 58%) as yellow solid.

### Step 2: Synthesis of 2-amino-4,6-bis(trifluoromethyl)phenol

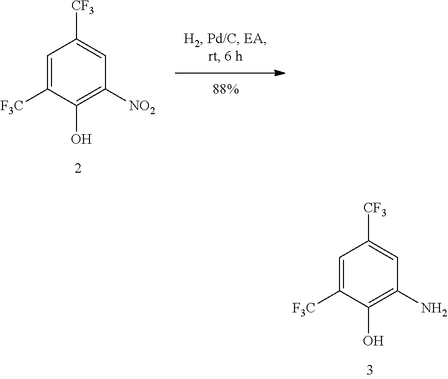 [SMILES: O=[N+]([O-])c1cc(C(F)(F)F)cc(C(F)(F)F)c1O ; Nc1cc(C(F)(F)F)cc(C(F)(F)F)c1O]

To a solution of compound 2 (1.4 g, 5.09 mmol, 1.0 eq) in EA (20 mL) was added Pd/C (280 mg, 20% wt) at rt. The reaction was stirred for 6 h at rt at 1 atm of hydrogen. After completion of the reaction by TLC (R_{f}=0.6, DCM/MeOH=10/1). The mixture was filtered with celite. The filtrate was concentrated under reduced pressure to afford compound 3 (1.1 g, 88%) as brown solid.

### Step 3: Synthesis of N,N-diallyl-2-(allyloxy)-3,5-bis(trifluoromethyl)aniline

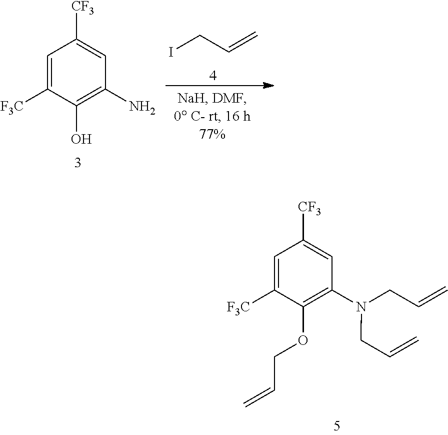

To a solution of compound 3 (2.11 g, 8.6 mmol, 1.0 eq) in DMF (10 mL) was added NaH (1.21 g, 30.1 mmol, 3.5 eq) at 0 °C. The reaction was stirred at rt for 30 mins. Compound 4 (5.7 g, 34.4 mmol, 4.0 eq) was added to above mixture. The reaction mixture was stirred at rt for 16 h. After completion of the reaction by TLC (R_{f}=0.3, PE/EA=30/1), the reaction solution was poured into water (30 mL). The solution was extracted with EA (50 mL×2). The combined organic layer was washed with brine, dried over Na₂SO₄ and filtered. The filtrate was concentrated under reduced pressure. The residue was purified by column chromatography (100-200 silica gel, 5% EA in PE as eluent) to afford compound 5 (2.42 g, 77%) as yellow oil.

### Step 4: Synthesis of 2-(diallylamino)-4,6-bis(trifluoromethyl)phenol

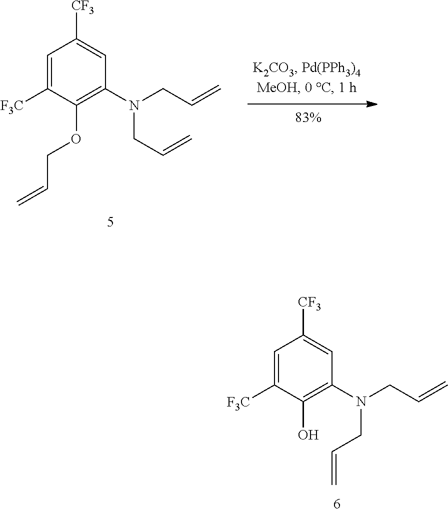 [SMILES: C=CCOc1c(N(CC=C)CC=C)cc(C(F)(F)F)cc1C(F)(F)F ; C=CCN(CC=C)c1cc(C(F)(F)F)cc(C(F)(F)F)c1O]

To a solution of compound 5 (2.42 g, 6.63 mmol, 1.0 eq) in MeOH (10 mL) were added K₂CO₃ (2.77 g, 19.8 mmol, 3.0 eq), Pd(PPh₃)₄ (767 mg, 0.663 mmol, 0.1 eq) at 0 °C. The reaction was stirred at 0 °C for 1 h. After completion of the reaction by TLC (R_{f}=0.1, PE/EA=30/1), the reaction solution was poured into water (30 mL). The mixture was extracted with EA (50 mL×2). The combined organic layer was washed with brine, dried over Na₂SO₄ and filtered. The filtration was concentrated under reduced pressure. The residue was purified by column chromatography (100-200 silica gel, 8.5% EA in PE as eluent) to afford compound 6 (1.8 g, 83%) as yellow oil.

### Step 5: Synthesis of 2-(1H-pyrrol-1-yl)-4,6-bis(trifluoromethyl)phenol

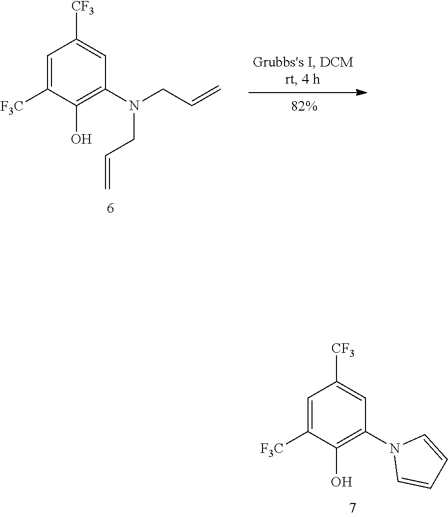 [SMILES: C=CCN(CC=C)c1cc(C(F)(F)F)cc(C(F)(F)F)c1O ; Oc1c(-n2cccc2)cc(C(F)(F)F)cc1C(F)(F)F]

To a solution of compound 6 (1.8 g, 5.53 mmol, 1.0 eq) in DCM (20 mL) was added Grubbs catalyst I (92 mg, 0.11 mmol, 0.02 eq) at rt. The reaction was stirred at rt for 4 h. After completion of the reaction by TLC (R_{f}=0.3, PE/EA=15/1), the reaction solution was poured into water (30 mL). The mixture was extracted with DCM (50 mL×2). The combined organic layer was washed with brine, dried over Na₂SO₄ and filtered. The filtrate was concentrated under reduced pressure. The residue was purified by column chromatography (100-200 silica gel, 12% EA in PE as eluent) to afford compound 7 (1.35 g, 82%) as yellow oil.

### Step 6: Synthesis of 2-(1H-pyrrol-1-yl)-4,6-bis(trifluoromethyl)phenyl (4-fluorophenyl)(methyl-d₃)carbamate

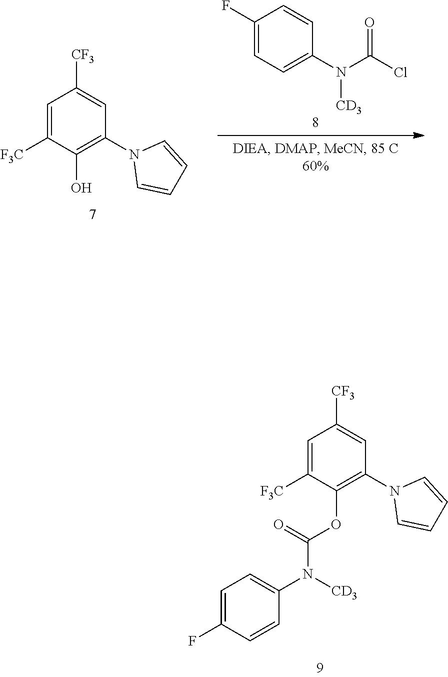 [SMILES: Oc1c(-n2cccc2)cc(C(F)(F)F)cc1C(F)(F)F ; [2H]C([2H])([2H])N(C(=O)Cl)c1ccc(F)cc1 ; [2H]C([2H])([2H])N(C(=O)Oc1c(-n2cccc2)cc(C(F)(F)F)cc1C(F)(F)F)c1ccc(F)cc1]

To a solution of compound 7 (757 mg, 2.56 mmol, 1.0 eq) in MeCN (10 mL) were added compound 8 (490 mg, 2.56 mmol, 1.0 eq), DIEA (828 mg, 6.41 mmol, 2.5 eq), DMAP (31 mg, 0.256 mmol, 0.1 eq) at rt. The reaction was stirred at 85 °C for 4 h. After completion of the reaction by TLC (R_{f}=0.6, PE/EA=15/1), the reaction solution was poured into water (30 mL). The mixture was extracted with EA (50 mL×2). The combined organic layer was washed with brine, dried over Na₂SO₄ and filtered. The filtrate was concentrated under reduced pressure. The residue was purified by column chromatography (100-200 silica gel, 8% EA in PE as eluent) to afford compound 9 (700 mg, 60%) as yellow oil.

### Step 7: Synthesis of 2-(2,5-dihydro-1H-pyrrol-1-yl)-4,6-bis(trifluoromethyl)phenyl (4-fluorophenyl)(methyl-d₃)carbamate

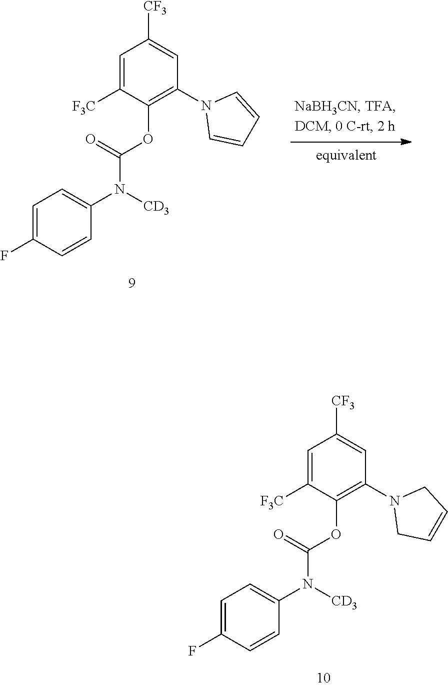 [SMILES: [2H]C([2H])([2H])N(C(=O)Oc1c(-n2cccc2)cc(C(F)(F)F)cc1C(F)(F)F)c1ccc(F)cc1 ; [2H]C([2H])([2H])N(C(=O)Oc1c(N2CC=CC2)cc(C(F)(F)F)cc1C(F)(F)F)c1ccc(F)cc1]

To a solution of compound 9 (200 mg, 0.445 mmol, 1.0 eq) in TFA/DCM (3 mL/3 mL) was added NaBH₃CN (29 mg, 0.445 mmol, 1.0 eq) at 0 °C. The reaction was stirred at rt for 2 h. After completion of the reaction by TLC (R_{f}=0.5, PE/EA=15/1), the reaction solution was poured into water (30 mL). The mixture was extracted with DCM (50 mL×2). The combined organic layer was washed with NaHCO₃ (sat. aq), brine, dried over Na₂SO₄ and filtered. The filtrate was concentrated under reduced pressure to afford compound 10 (250 mg, crude) as yellow oil.

### Step 8: Synthesis of 2-((3S,4R)-3,4-dihydroxypyrrolidin-1-yl)-4,6-bis(trifluoromethyl)phenyl (4-fluorophenyl)(methyl-d₃)carbamate

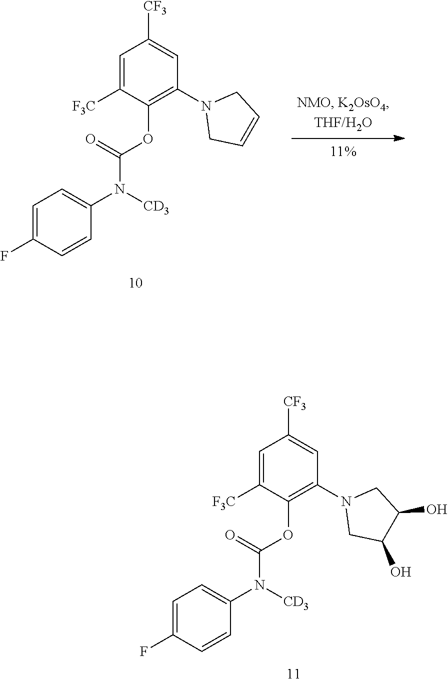 [SMILES: [2H]C([2H])([2H])N(C(=O)Oc1c(N2CC=CC2)cc(C(F)(F)F)cc1C(F)(F)F)c1ccc(F)cc1 ; [2H]C([2H])([2H])N(C(=O)Oc1c(N2C[C@@H](O)[C@@H](O)C2)cc(C(F)(F)F)cc1C(F)(F)F)c1ccc(F)cc1]

To a solution of compound 10 (250 mg, 0.55 mmol, 1.0 eq) in t-BuOH/THF/H₂O (3 mL/3 mL/1.5 mL) at rt were added NMO (91 mg, 0.77 mmol, 1.4 eq), K₂OsO₄ (6 mg, 0.017 mmol, 0.03 eq). The mixture was stirred for 4 h at rt. After completion of the reaction checked by TLC (R_{f11}=0.4, PE/EA=2/1), the mixture was poured into water (30 mL) to quench the reaction. Then the mixture was extracted with EA (50 mL×2). The combined organic layer was washed with brine, dried over Na₂SO₄ and filtered. The filtration was concentrated under reduced pressure. The residue was purified prep-HPLC to give title compound (11; 32 mg, 11%) as white solid.

¹H NMR (400 MHz, CDCl₃) δ 7.29-7.23 (m, 3H), 7.12-6.99 (m, 3H), 4.30 (br, 2H), 3.68-3.35 (m, 2H), 2.85-2.75 (m, 4H); LC-MS: 486.10 [M+H]⁺.

### Example 42

### 2-(3-(2-(1H-imidazol-1-yl)ethyl)-2-oxoimidazolidin-1-yl)-4,6-bis(trifluoromethyl)phenyl (4-fluorophenyl)(methyl-d₃)carbamate

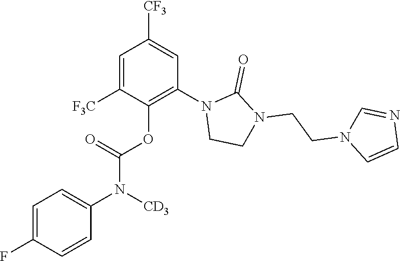 [SMILES: [2H]C([2H])([2H])N(C(=O)Oc1c(N2CCN(CCn3ccnc3)C2=O)cc(C(F)(F)F)cc1C(F)(F)F)c1ccc(F)cc1]

### Step 1: Synthesis of 1-(2-hydroxy-3,5-bis(trifluoromethyl)phenyl)-3-(2-hydroxyethyl)imidazolidin-2-one

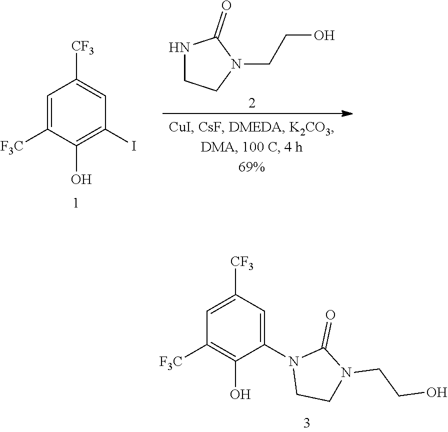 [SMILES: Oc1c(I)cc(C(F)(F)F)cc1C(F)(F)F ; O=C1NCCN1CCO ; O=C1N(CCO)CCN1c1cc(C(F)(F)F)cc(C(F)(F)F)c1O]

To a solution of compound 1 (2.0 g, 5.62 mmol, 1.0 eq) and compound 2 (1.1 g, 8.4 mmol, 1.5 eq) in DMA (5 mL) were added CuI (534 mg, 2.81 mmol, 0.5 eq), DMEDA (490 mg, 5.62 mmol, 1.0 eq), K₂CO₃ (15.6 g, 11.2 mmol, 2.0 eq) and CsF (1.7 g, 11.24 mmol, 2.0 eq) at rt. The reaction mixture was stirred at 100 °C in an oil-bath for 4 h. After completion of the reaction checked by TLC (R_{f} 0.2, PE:EA=1:1), water (50 mL) was added and adjusted to pH 6 with HCl (1 N). The mixture was extracted with EA (50 mL×2). The combined organic layer was washed with brine, dried over Na₂SO₄ and filtered. The filtrate was concentrated under reduced pressure. The residue was purified by column chromatography (100-200 silica gel, 70% EA in PE as eluent) to afford compound 3 (1.38 g, 69%) as pale-green solid.

### Step 2: Synthesis of 1-(2-benzyloxy-3,5-bis(trifluoromethyl)phenyl)-3-(2-hydroxyethyl)imidazolidin-2-one

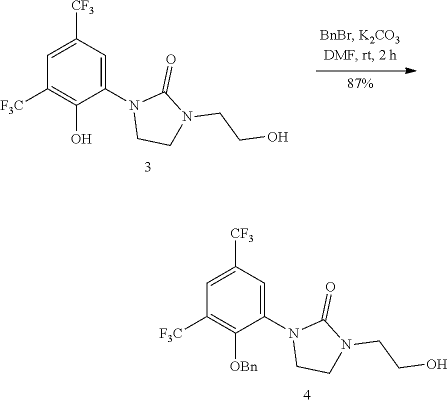 [SMILES: O=C1N(CCO)CCN1c1cc(C(F)(F)F)cc(C(F)(F)F)c1O ; O=C1N(CCO)CCN1c1cc(C(F)(F)F)cc(C(F)(F)F)c1OCc1ccccc1]

To a solution of compound 3 (2.15 g, 6.0 mmol, 1.0 eq) in DMF (10 mL) at rt were added K₂CO₃ (1.09 g, 7.8 mmol, 1.3 eq) and BnBr (1.23 g, 7.2 mmol, 1.2 eq). The reaction was stirred at rt for 2 h. After completion of the reaction monitored by TLC (R_{f}=0.3, PE:EA=1:1), the mixture was extracted with EA (50 mL×2). The combined organic layer was washed with brine, dried over Na₂SO₄ and filtered. The filtration was concentrated under reduced pressure. The residue was purified by column chromatography (100-200 silica gel, 20% EA in PE as eluent) to afford compound 4 (1.89 g, 87%) as off-white solid.

### Step 3: Synthesis of 1-(2-benzyloxy-3,5-bis(trifluoromethyl)phenyl)-3-((tert-butyldimethylsilyl)oxy)ethyl)imidazolidin-2-one

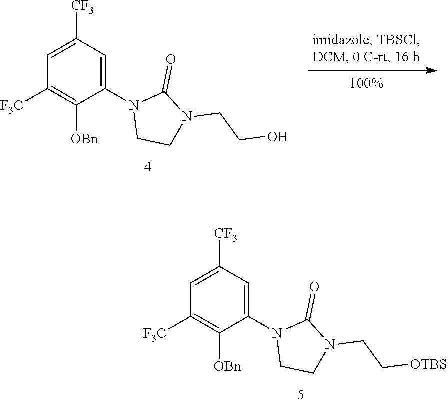 [SMILES: O=C1N(CCO)CCN1c1cc(C(F)(F)F)cc(C(F)(F)F)c1OCc1ccccc1 ; CC(C)(C)[Si](C)(C)OCCN1CCN(c2cc(C(F)(F)F)cc(C(F)(F)F)c2OCc2ccccc2)C1=O]

To a solution of compound 4 (1.89 g, 4.2 mmol, 1.0 eq) in DCM (10 mL) at 0 C were added imidazole (580 mg, 8.43 mmol, 2.0 eq) and TBSCl (1.27 g, 8.43 mmol, 2.0 eq). The reaction was stirred at rt for 16 h. After completion of the reaction by TLC (R_{f} 0.9, PE:EA=1:1), the reaction solution was poured into water (30 mL). The mixture was extracted with EA (50 mL×2). The combined organic layer was washed with brine, dried over Na₂SO₄ and filtered. The filtrate was concentrated under reduced pressure. The residue was purified by column chromatography (100-200 silica gel, 6% EA in PE as eluent) to afford compound 5 as pale-yellow oil (2.15 g, 100%).

### Step 4: Synthesis of 1-(2-hydroxy-3,5-bis(trifluoromethyl)phenyl)-3-((tert-butyldimethylsilyl)oxy)ethyl)imidazolidin-2-one

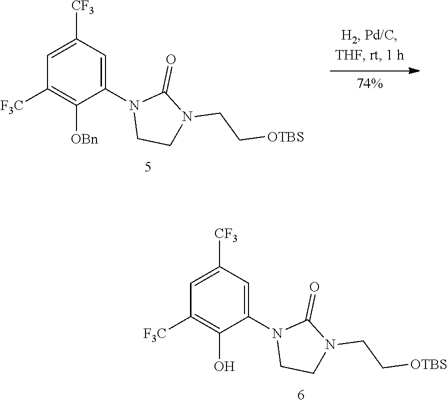 [SMILES: CC(C)(C)[Si](C)(C)OCCN1CCN(c2cc(C(F)(F)F)cc(C(F)(F)F)c2OCc2ccccc2)C1=O ; CC(C)(C)[Si](C)(C)OCCN1CCN(c2cc(C(F)(F)F)cc(C(F)(F)F)c2O)C1=O]

To a solution of compound 5 (2.12 g, 3.73 mmol, 1.0 eq) in THF (5 mL) was added Pd/C (250 mg) at rt. The reaction was stirred for 1 h under H₂ atmosphere. After completion of the reaction checked by TLC (R_{f} 0.6, PE:EA=5:1), the mixture was filtered and washed with EA (20 mL×2). The solution was concentrated to give compound 6 (1.58 g, 74%) as off-white solid.

### Step 5: Synthesis of 2-(3-(2-((tert-butyldimethylsiliil)oxy)ethyl)-2-oxoimidazolidin-1-yl)-4,6-bis(trifluoromethyl)phenyl (4-fluorophenyl)(methyl-d₃)carbamate

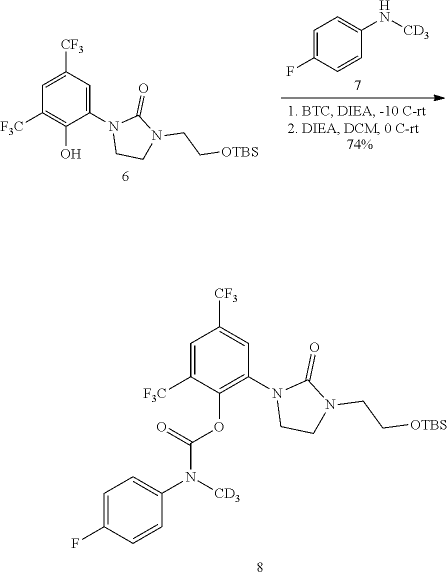 [SMILES: CC(C)(C)[Si](C)(C)OCCN1CCN(c2cc(C(F)(F)F)cc(C(F)(F)F)c2O)C1=O ; [2H]C([2H])([2H])Nc1ccc(F)cc1 ; [2H]C([2H])([2H])N(C(=O)Oc1c(N2CCN(CCO[Si](C)(C)C(C)(C)C)C2=O)cc(C(F)(F)F)cc1C(F)(F)F)c1ccc(F)cc1]

To a solution of compound 6 (500 mg, 1.05 mmol, 1.0 eq) in DCM (10 mL) at −10 °C were added DIEA (410 mg, 3.17 mmol, 3.0 eq) and a solution of triphosgene (141 mg, 0.476 mmol, 0.45 eq) in DCM (3 mL). The reaction mixture was stirred for 10 min at rt under N2. The reaction solution was concentrated under reduced pressure to remove the solvent. Then DCM (3 mL) was added to above residue. The solution was used for next step.

To a solution of compound 7 (196 mg, 1.58 mmol, 1.5 eq) in DCM (5 mL) at 0 °C was added the above solution prepared above. The mixture was stirred for 1 h at rt under N2. After completion of the reaction monitored by TLC (R_{f} 0.5, PE:EA=5:1), HCl (1 M, 15 mL) was added. Then the mixture was extracted with DCM (20 mL×2). The combined organic layer was washed with brine, dried over Na₂SO₄ and filtered. The filtrate was concentrated under reduced pressure. The residue was purified with FCC to give compound 8 (370 mg, 74%) as off-yellow oil.

### Step 6: Synthesis of 2-(3-(2-hydroxyethyl)-2-oxoimidazolidin-1-yl)-4,6-bis(trifluoromethyl)phenyl (4-fluorophenyl)(methyl-d₃)carbamate

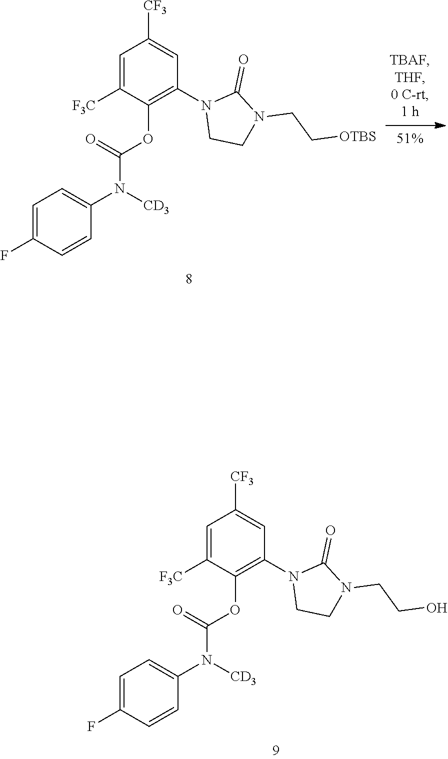 [SMILES: [2H]C([2H])([2H])N(C(=O)Oc1c(N2CCN(CCO[Si](C)(C)C(C)(C)C)C2=O)cc(C(F)(F)F)cc1C(F)(F)F)c1ccc(F)cc1 ; [2H]C([2H])([2H])N(C(=O)Oc1c(N2CCN(CCO)C2=O)cc(C(F)(F)F)cc1C(F)(F)F)c1ccc(F)cc1]

To a solution of compound 8 (350 mg, 0.561 mmol, 1.0 eq) in THF (10 mL) at 0 °C was added TBAF (1M, 1.68 mL, 1.68 mmol, 1.5 eq). The reaction was stirred at rt for 1 h. After completion of the reaction checked by TLC (R_{f} 0.2, PE:EA=1:1), the mixture was diluted with water (30 mL), extracted with EA (30 mL×2). The combined organic layer was washed with brine, dried over Na₂SO₄ and filtered. The filtrate was concentrated under reduced pressure. The residue was purified by column chromatography (100-200 silica gel, 70 EA in PE as eluent) to afford compound 9 (180 mg, 51%) as pale-yellow oil.

### Step 7: Synthesis of 2-(3-(2-(((4-fluorophenyl)(methyl-d₃)carbamoyl)oxy)-3,5-bis(trifluoromethyl)phenyl)-2-oxoimidazolidin-1-yl)ethyl methanesulfonate

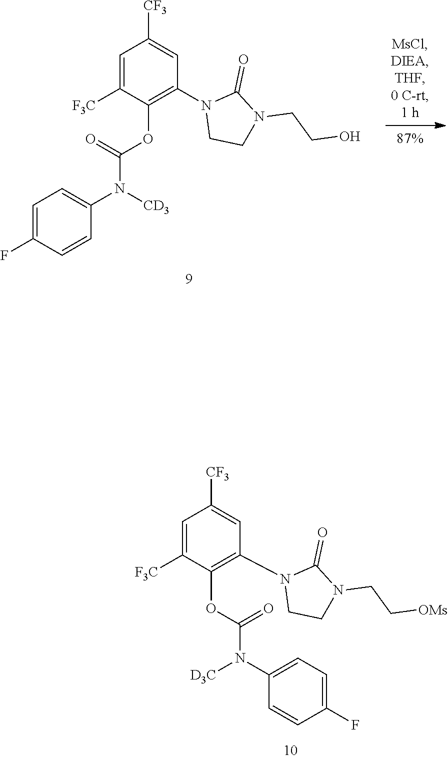 [SMILES: [2H]C([2H])([2H])N(C(=O)Oc1c(N2CCN(CCO)C2=O)cc(C(F)(F)F)cc1C(F)(F)F)c1ccc(F)cc1 ; [2H]C([2H])([2H])N(C(=O)Oc1c(N2CCN(CCOS(C)(=O)=O)C2=O)cc(C(F)(F)F)cc1C(F)(F)F)c1ccc(F)cc1]

To a solution of compound 9 (100 mg, 0.196 mmol, 1.0 eq) in THF (10 mL) at 0 °C were added DIEA (76 mg, 0.589 mmol, 3.0 eq) and MsCl (68 mg, 0.589 mmol, 3.0 eq). The reaction was stirred at rt for 1 h. After completion of the reaction by TLC (R_{f} 0.6, DCM:MeOH=10:1), the mixture was extracted with EA (30 mL×2). The combined organic layer was washed with brine, dried over Na₂SO₄ and filtered. The filtrate was concentrated under reduced pressure. The residue was purified by column chromatography (100-200 silica gel, 6% MeOH in DCM as eluent) to afford compound (87 mg, 87%) as pale-yellow solid.

### Step 8: Synthesis of 2-(3-(2-(1H-imidazol-1-yl)ethyl)-2-oxoimidazolidin-1-yl)-4,6-bis(trifluoromethyl)phenyl (4-fluorophenyl)(methyl-d₃)carbamate

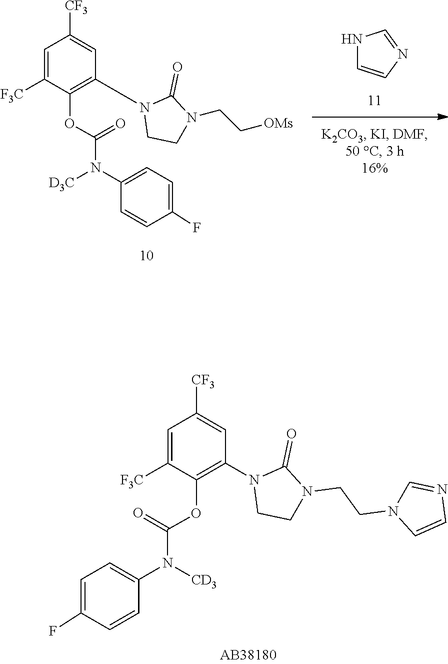 [SMILES: [2H]C([2H])([2H])N(C(=O)Oc1c(N2CCN(CCOS(C)(=O)=O)C2=O)cc(C(F)(F)F)cc1C(F)(F)F)c1ccc(F)cc1 ; c1c[nH]cn1 ; [2H]C([2H])([2H])N(C(=O)Oc1c(N2CCN(CCn3ccnc3)C2=O)cc(C(F)(F)F)cc1C(F)(F)F)c1ccc(F)cc1]

To a solution of compound 10 (147 mg, 0.249 mmol, 1.0 eq) in DMF (5 mL) at rt were added compound 11 (46 mg, 0.677 mmol, 2.0 eq), K₂CO₃ (104 mg, 0.747 mmol, 3.0 eq), KI (13 mg, 0.0747 mmol, 0.3 eq). The reaction was heated at 50 °C for 3 h. After completion of the reaction monitored by TLC (R_{f} 0.4, DCM:MeOH=10:1), the mixture diluted with EA (60 mL), and washed with brine, dried over Na₂SO₄ and filtered. The filtrate was concentrated under reduced pressure. The residue was purified by Prep-HPLC to afford the title compound (AB38180; 30.4 mg, 16%) as white solid.

¹H NMR (400 MHz, CD₃OD): δ 8.97-8.94 (m, 1H), 7.94-7.84 (m, 2H), 7.37-7.68 (m, 4H), 7.14-7.16 (t, 2H), 4.47 (t, 2H), 3.75-3.63 (m, 6H); LC-MS: 563.25 [M+H]⁻.

### Example 43

### 2-(3-(2-(4H-1,2,4-triazol-4-yl)ethyl)-2-oxoimidazolidin-1-yl)-4,6-bis(trifluoromethyl)phenyl (4-fluorophenyl)(methyl-d₃)carbamate

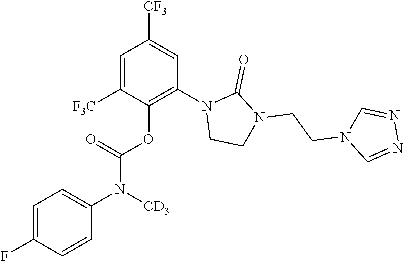 [SMILES: [2H]C([2H])([2H])N(C(=O)Oc1c(N2CCN(CCn3cnnc3)C2=O)cc(C(F)(F)F)cc1C(F)(F)F)c1ccc(F)cc1]

### Step 1: Synthesis of 1-(2-hydroxy-3,5-bis(trifluoromethyl)phenyl)-3-(2-hydroxyethyl)imidazolidine-2-one

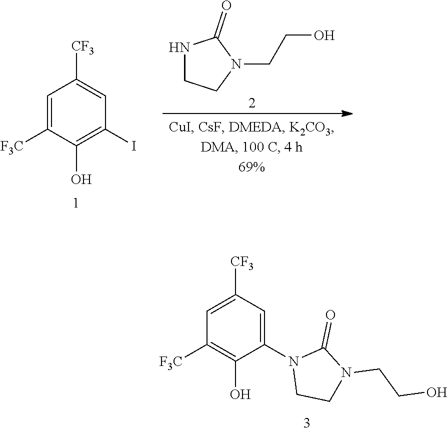 [SMILES: Oc1c(I)cc(C(F)(F)F)cc1C(F)(F)F ; O=C1NCCN1CCO ; O=C1N(CCO)CCN1c1cc(C(F)(F)F)cc(C(F)(F)F)c1O]

To a solution of compound 1 (2.0 g, 5.62 mmol, 1.0 eq) and compound 2 (1.1 g, 8.4 mmol, 1.5 eq) in DMA (5 mL) were added CuI (534 mg, 2.81 mmol, 0.5 eq), DMEDA (490 mg, 5.62 mmol, 1.0 eq), K₂CO₃ (15.6 g, 11.2 mmol, 2.0 eq) and CsF (1.7 g, 11.24 mmol, 2.0 eq) at rt. The reaction mixture was stirred at 100 °C in an oil-bath for 4 h. After completion of the reaction checked by TLC (R_{f} 0.2, PE:EA=1:1), water (50 mL) was added and adjusted to pH 6 with HCl (1 N). The mixture was extracted with EA (50 mL×2). The combined organic layer was washed with brine, dried over Na₂SO₄ and filtered. The filtrate was concentrated under reduced pressure. The residue was purified by column chromatography (100-200 silica gel, 70% EA in PE as eluent) to afford compound 3 (1.38 g, 69%) as pale-green solid.

### Step 2: Synthesis of 1-(2-benzyloxy-3,5-bis(trifluoromethyl)phenyl)-3-(2-hydroxyethyl)imidazolidine-2-one

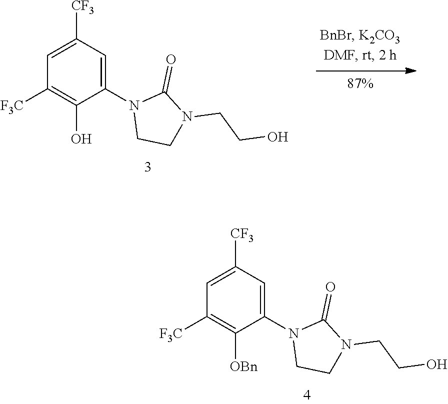 [SMILES: O=C1N(CCO)CCN1c1cc(C(F)(F)F)cc(C(F)(F)F)c1O ; O=C1N(CCO)CCN1c1cc(C(F)(F)F)cc(C(F)(F)F)c1OCc1ccccc1]

To a solution of compound 3 (2.15 g, 6.0 mmol, 1.0 eq) in DMF (10 mL) at rt were added K₂CO₃ (1.09 g, 7.8 mmol, 1.3 eq) and BnBr (1.23 g, 7.2 mmol, 1.2 eq). The reaction was stirred at rt for 2 h. After completion of the reaction monitored by TLC (R_{f}=0.3, PE:EA=1:1), the mixture was extracted with EA (50 mL×2). The combined organic layer was washed with brine, dried over Na₂SO₄ and filtered. The filtration was concentrated under reduced pressure. The residue was purified by column chromatography (100-200 silica gel, 20% EA in PE as eluent) to afford compound 4 (1.89 g, 87%) as off-white solid.

### Step 3: Synthesis of 1-(2-benzyloxy-3,5-bis(trifluoromethyl)phenyl)-3-(2-((tert-butyldimethylsilyl)oxy)ethyl)imidazolidine-2-one

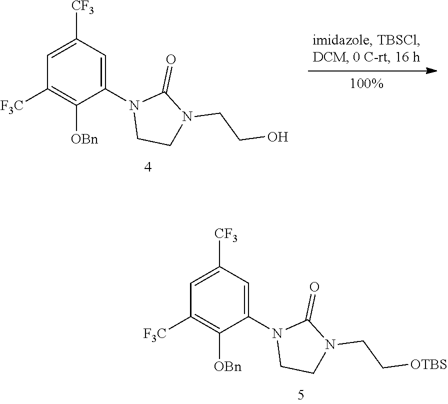 [SMILES: O=C1N(CCO)CCN1c1cc(C(F)(F)F)cc(C(F)(F)F)c1OCc1ccccc1 ; CC(C)(C)[Si](C)(C)OCCN1CCN(c2cc(C(F)(F)F)cc(C(F)(F)F)c2OCc2ccccc2)C1=O]

To a solution of compound 4 (1.89 g, 4.2 mmol, 1.0 eq) in DCM (10 mL) at 0 C were added imidazole (580 mg, 8.43 mmol, 2.0 eq) and TBSCl (1.27 g, 8.43 mmol, 2.0 eq). The reaction was stirred at rt for 16 h. After completion of the reaction by TLC (R_{f} 0.9, PE:EA=1:1), the reaction solution was poured into water (30 mL). The mixture was extracted with EA (50 mL×2). The combined organic layer was washed with brine, dried over Na₂SO₄ and filtered. The filtrate was concentrated under reduced pressure. The residue was purified by column chromatography (100-200 silica gel, 6% EA in PE as eluent) to afford compound 5 as pale-yellow oil (2.15 g, 100%).

### Step 4: Synthesis of 1-(2-hydroxy-3,5-bis(trifluoromethyl)phenyl)-3-(2-((tert-butyldimethylsilyl)oxy)ethyl)imidazolidine-2-one

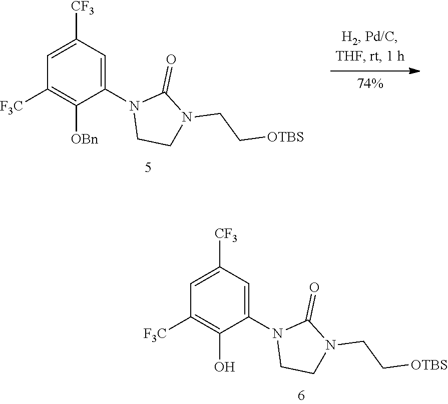 [SMILES: CC(C)(C)[Si](C)(C)OCCN1CCN(c2cc(C(F)(F)F)cc(C(F)(F)F)c2OCc2ccccc2)C1=O ; CC(C)(C)[Si](C)(C)OCCN1CCN(c2cc(C(F)(F)F)cc(C(F)(F)F)c2O)C1=O]

To a solution of compound 5 (2.12 g, 3.73 mmol, 1.0 eq) in THF (5 mL) was added Pd/C (250 mg) at rt. The reaction was stirred for 1 h under H₂ atmosphere. After completion of the reaction checked by TLC (R_{f} 0.6, PE:EA=5:1), the mixture was filtered and washed with EA (20 mL×2). The solution was concentrated to give compound 6 (1.58 g, 74%) as off-white solid.

### Step 5: Synthesis of 2-(3-(2-((tert-butyldimethylsilyl)oxy)ethyl)-2-oxoimadizolidin-1-yl)-4,6-bis(trifluoromethyl)phenyl (4-fluorophenyl)(methyl-d₃)carbamate

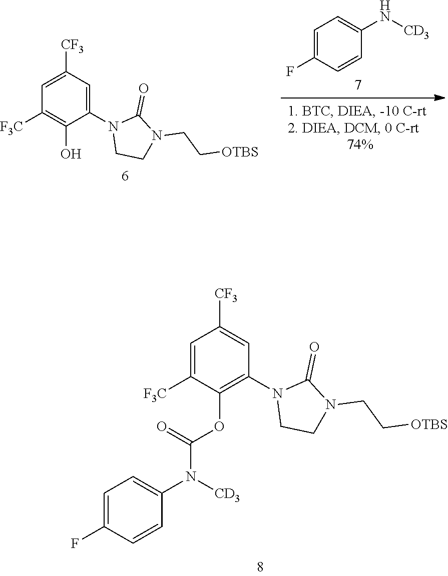 [SMILES: CC(C)(C)[Si](C)(C)OCCN1CCN(c2cc(C(F)(F)F)cc(C(F)(F)F)c2O)C1=O ; [2H]C([2H])([2H])Nc1ccc(F)cc1 ; [2H]C([2H])([2H])N(C(=O)Oc1c(N2CCN(CCO[Si](C)(C)C(C)(C)C)C2=O)cc(C(F)(F)F)cc1C(F)(F)F)c1ccc(F)cc1]

To a solution of compound 6 (500 mg, 1.05 mmol, 1.0 eq) in DCM (10 mL) at −10 °C were added DIEA (410 mg, 3.17 mmol, 3.0 eq) and a solution of triphosgene (141 mg, 0.476 mmol, 0.45 eq) in DCM (3 mL). The reaction mixture was stirred for 10 min at rt under N2. The reaction solution was concentrated under reduced pressure to remove the solvent. Then DCM (3 mL) was added to above residue. The solution was used for next step.

To a solution of compound 7 (196 mg, 1.58 mmol, 1.5 eq) in DCM (5 mL) at 0 °C was added the above solution prepared above. The mixture was stirred for 1 h at rt under N2. After completion of the reaction monitored by TLC (R_{f} 0.5, PE:EA=5:1), HCl (1 M, 15 mL) was added. Then the mixture was extracted with DCM (20 mL×2). The combined organic layer was washed with brine, dried over Na₂SO₄ and filtered. The filtrate was concentrated under reduced pressure. The residue was purified with FCC to give compound 8 (370 mg, 74%) as off-yellow oil.

### Step 6: Synthesis of 2-(3-(2-hydroxyethyl)-2-oxoimadizolidin-1-yl)-4,6-bis(trifluoromethyl)phenyl (4-fluorophenyl)(methyl-d₃)carbamate

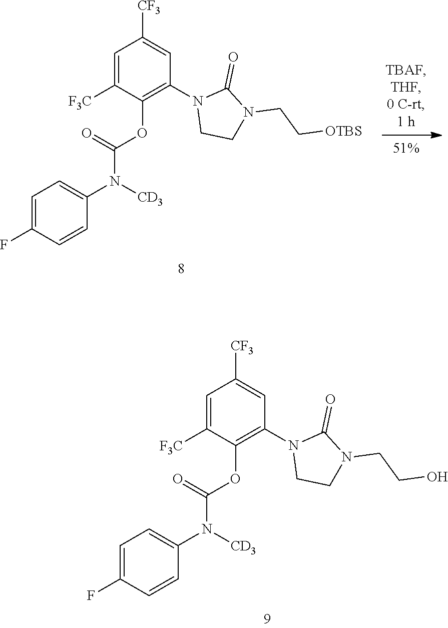 [SMILES: [2H]C([2H])([2H])N(C(=O)Oc1c(N2CCN(CCO[Si](C)(C)C(C)(C)C)C2=O)cc(C(F)(F)F)cc1C(F)(F)F)c1ccc(F)cc1 ; [2H]C([2H])([2H])N(C(=O)Oc1c(N2CCN(CCO)C2=O)cc(C(F)(F)F)cc1C(F)(F)F)c1ccc(F)cc1]

To a solution of compound 8 (350 mg, 0.561 mmol, 1.0 eq) in THF (10 mL) at 0 °C was added TBAF (1M, 1.68 mL, 1.68 mmol, 1.5 eq). The reaction was stirred at rt for 1 h. After completion of the reaction checked by TLC (R_{f} 0.2, PE:EA=1:1), the mixture was diluted with water (30 mL), extracted with EA (30 mL×2). The combined organic layer was washed with brine, dried over Na₂SO₄ and filtered. The filtrate was concentrated under reduced pressure. The residue was purified by column chromatography (100-200 silica gel, 70 EA in PE as eluent) to afford compound 9 (180 mg, 51%) as pale-yellow oil.

### Step 7: Synthesis of 2-(3-(2-(4H-1,2,4-triazol-4-yl)ethyl)-2-oxoimidazolidin-1-yl)-4,6-bis(trifluoromethyl)phenyl (4-fluorophenyl)(methyl-d₃)carbamate

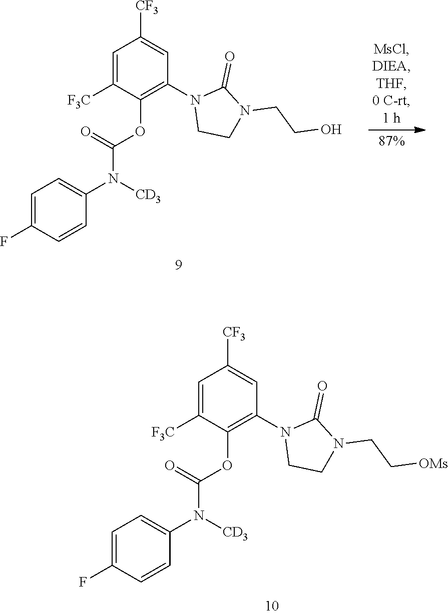 [SMILES: [2H]C([2H])([2H])N(C(=O)Oc1c(N2CCN(CCO)C2=O)cc(C(F)(F)F)cc1C(F)(F)F)c1ccc(F)cc1 ; [2H]C([2H])([2H])N(C(=O)Oc1c(N2CCN(CCOS(C)(=O)=O)C2=O)cc(C(F)(F)F)cc1C(F)(F)F)c1ccc(F)cc1]

To a solution of compound 9 (100 mg, 0.196 mmol, 1.0 eq) in THF (10 mL) at 0 °C were added DIEA (76 mg, 0.589 mmol, 3.0 eq) and MsCl (68 mg, 0.589 mmol, 3.0 eq). The reaction was stirred at rt for 1 h. After completion of the reaction by TLC (R_{f} 0.6, DCM:MeOH=10:1), the mixture was extracted with EA (30 mL×2). The combined organic layer was washed with brine, dried over Na₂SO₄ and filtered. The filtrate was concentrated under reduced pressure. The residue was purified by column chromatography (100-200 silica gel, 6% MeOH in DCM as eluent) to afford compound 10 (87 mg, 87%) as pale-yellow solid.

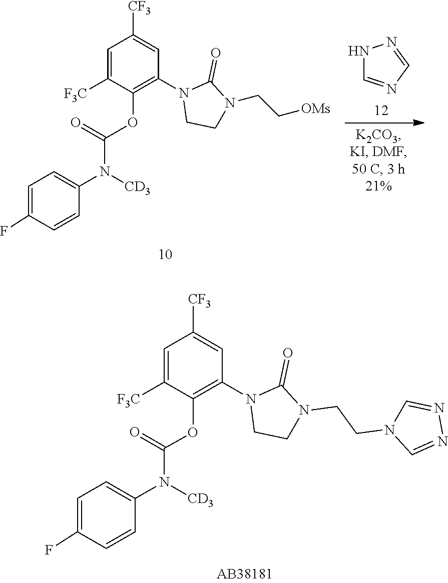 [SMILES: [2H]C([2H])([2H])N(C(=O)Oc1c(N2CCN(CCOS(C)(=O)=O)C2=O)cc(C(F)(F)F)cc1C(F)(F)F)c1ccc(F)cc1 ; c1nc[nH]n1 ; [2H]C([2H])([2H])N(C(=O)Oc1c(N2CCN(CCn3cnnc3)C2=O)cc(C(F)(F)F)cc1C(F)(F)F)c1ccc(F)cc1]

To a solution of compound 10 (147 mg, 0.249 mmol, 1.0 eq) in DMF (5 mL) at rt were added compound 12 (47 mg, 0.677 mmol, 1.5 eq), K₂CO₃ (104 mg, 0.747 mmol, 3.0 eq), KI (13 mg, 0.0747 mmol, 0.3 eq). The reaction was heated at 50 °C for 3 h. After completion of the reaction checked with TLC (R_{f} 0.4, DCM:MeOH=10:1), the mixture was diluted with EA (60 mL), washed with brine, dried with Na₂SO₄ and filtered. The filtrate was concentrated under reduced pressure. The residue was purified by Prep-HPLC to afford title compound (AB38181; 42.1 mg, 22%) as white solid.

¹H NMR (400 MHz, CD₃OD): δ 8.69-8.67 (m, 1H), 8.10-7.82 (m, 3H), 7.34-7.36 (m, 2H), 7.13-7.17 (t, 2H), 4.49 (4.2H), 3.75-3.48 (m, 6H); LC-MS: 564.30 [M+H]⁻.

### Example 44

### 2-(3-(2-hydroxy-3-(methylamino)propyl)-2-oxoimidazolidin-1-yl)-4,6-bis(trifluoromethyl)phenyl (3-chloro-2,4-difluorophenyl)(methyl-d₃)carbamate

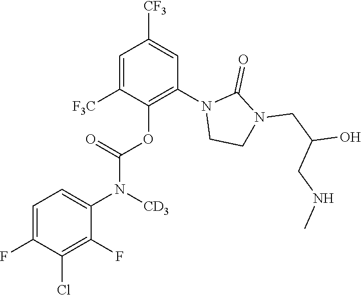 [SMILES: [2H]C([2H])([2H])N(C(=O)Oc1c(N2CCN(CC(O)CNC)C2=O)cc(C(F)(F)F)cc1C(F)(F)F)c1ccc(F)c(Cl)c1F]

To a solution of 2-(3-(oxiran-2-ylmethyl)-2-oxoimidazolidin-1-yl)-4,6-bis(trifluoromethyl)phenyl (3-chloro-2,4-difluorophenyl)(methyl-d₃)carbamate (0.050 g, 0.086 mmol) in 3 mL anhydrous methanol at 0 °C. 2M methylamine (1 mL, 1 mmol) was added. The reaction was stirred for 20 h at room temperature. The solvent was removed under reduced pressure. The oil was dissolved in dichloromethane and triturated with hexanes. The solvent was removed under reduced pressure to afford title compound as semi solid (15 mg, 48% yield).

¹H NMR (400 MHz, CDCl₃) δ 7.8 (m, 1H), 7.76 (m, 1H), 7.08 (m, 2H), 3.92 (m, 1H),), 3.67 (m, 2H), 3.10 (m, 2H), 2.91 (m, 2H), 2.65 (m, 2H), 1.25 (m, 3H); ESIMS: m/z 608.22.24 [(M+H)⁺].

### Example 45

### 2-(3-(2-hydroxy-3-(methylamino)propyl)-2-oxoimidazolidin-1-yl)-4,6-bis(trifluoromethyl)phenyl (4-fluorophenyl)(methyl-d₃)carbamate

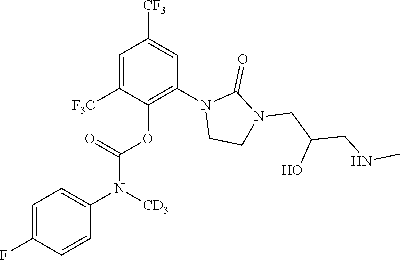 [SMILES: [2H]C([2H])([2H])N(C(=O)Oc1c(N2CCN(CC(O)CNC)C2=O)cc(C(F)(F)F)cc1C(F)(F)F)c1ccc(F)cc1]

### Step 1: Synthesis of 1-(2-hydroxy-3,5-bis(trifluoromethyl)phenyl)imidazolidine-2-one

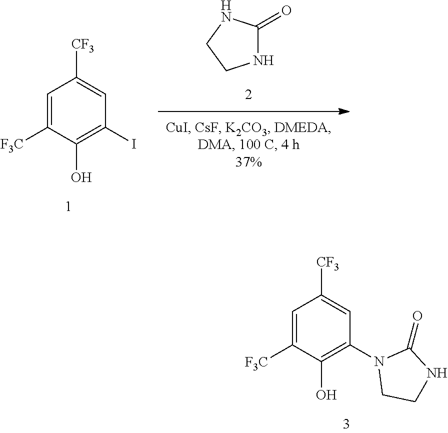 [SMILES: Oc1c(I)cc(C(F)(F)F)cc1C(F)(F)F ; O=C1NCCN1 ; O=C1NCCN1c1cc(C(F)(F)F)cc(C(F)(F)F)c1O]

To a solution of compound 1 (3.0 g, 8.45 mmol, 1.0 eq) and compound 2 (1.45 g, 16.9 mmol, 2.0 eq) in DMA (15 mL) were added CuI (803 mg, 4.23 mmol, 0.5 eq), DMEDA (744 mg, 8.45 mmol, 1.0 eq), K₂CO₃ (2.35 g, 16.9 mmol, 2.0 eq) and CsF (2.57 g, 16.9 mmol, 2.0 eq) at rt. The reaction mixture was stirred at 100 °C in an oil-bath for 4 h. After completion of the reaction by TLC (R_{f}=0.2, PE/EtOAc=3/1), water (50 mL) was added and adjust to pH 6 with 1N HCL. The mixture was extracted with EtOAc (100 mL×2). The combined organic layer was washed with brine, dried over Na₂SO₄ and filtered. The filtration was concentrated under reduced pressure. The residue was purified by column chromatography (100-200 silica gel, 40% EtOAc in PE as eluent) to afford compound 3 as pale green solid (1.0 g, 37%).

### Step 2: Synthesis of tert-butyl 3-(2-hydroxy-3,5-bis(trifluoromethyl)phenyl)-2-oxoimidazolidine-1-carboxylate

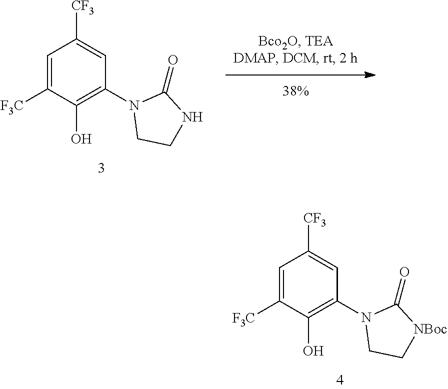 [SMILES: O=C1NCCN1c1cc(C(F)(F)F)cc(C(F)(F)F)c1O ; CC(C)(C)OC(=O)N1CCN(c2cc(C(F)(F)F)cc(C(F)(F)F)c2O)C1=O]

To a solution of compound 3 (1.0 g, 3.18 mmol, 1.0 eq) in DMF (10 mL) were added TEA (386 mg, 3.82 mmol, 1.2 eq), DMAP (40 mg, 0.318 mmol, 0.1 eq) and Boc₂O (728 mg, 3.50 mmol, 1.05 eq) at rt. The reaction was stirred for 2 h at rt. After completion of the reaction by TLC (R_{f}=0.4, PE/EtOAc=3/1). The mixture was extracted with EtOAc (50 mL×2). The combined organic layer was washed with brine, dried over Na₂SO₄ and filtered. The filtration was concentrated under reduced pressure. The residue was purified by column chromatography (100-200 silica gel, 35% EtOAc in PE as eluent) to afford compound 4 (500 mg, 38%) as off-white solid.

### Step 3: Synthesis of tert-butyl 3-(2-benzyloxy-3,5-bis(trifluoromethyl)phenyl)-2-oxoimidazolidine-1-carboxylate

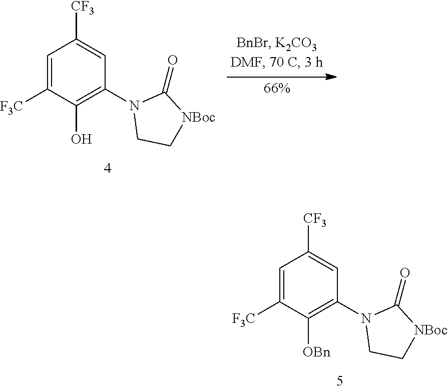 [SMILES: CC(C)(C)OC(=O)N1CCN(c2cc(C(F)(F)F)cc(C(F)(F)F)c2O)C1=O ; CC(C)(C)OC(=O)N1CCN(c2cc(C(F)(F)F)cc(C(F)(F)F)c2OCc2ccccc2)C1=O]

To a solution of compound 4 (566 mg, 1.37 mmol, 1.0 eq) in DCM (10 mL) were added K₂CO₃ (286 mg, 2.05 mmol, 1.5 eq) and BnBr (351 mg, 2.05 mmol, 1.5 eq) at rt. The reaction was stirred at 70 °C for 3 h. After completion of the reaction by TLC (Rf=0.9, PE/EtOAc=3/1), the reaction solution was poured into water (30 mL). The mixture was extracted with EtOAc (50 mL×2). The combined organic layer was washed with brine, dried over Na₂SO₄ and filtered. The filtration was concentrated under reduced pressure. The residue was purified by column chromatography (100-200 silica gel, 5% EtOAc in PE as eluent) to afford compound 5 as pale yellow solid (460 mg, 66%).

### Step 4: Synthesis of 1-(2-benzyloxy-3,5-bis(trifluoromethyl)phenyl)-2-oxoimidazolidine

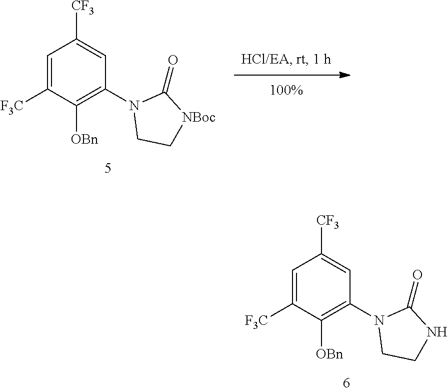 [SMILES: CC(C)(C)OC(=O)N1CCN(c2cc(C(F)(F)F)cc(C(F)(F)F)c2OCc2ccccc2)C1=O ; O=C1NCCN1c1cc(C(F)(F)F)cc(C(F)(F)F)c1OCc1ccccc1]

Compound 5 (460 mg, 0.91 mmol, 1.0 eq) was added into HCl/dioxane (4 M, 5 mL) at 0 °C. The reaction was stirred for 1 h at rt. After completion of the reaction by TLC (R_{f}=0.3, PE/EtOAc=3/1), the mixture was concentrated to give compound 6 (405 mg, 100%) as off-white solid.

### Step 5: Synthesis of 1-(2-(benzyloxy)-3,5-bis(trifluoromethyl)phenyl)-3-(oxiran-2-ylmethyl)imidazolidine-2-one

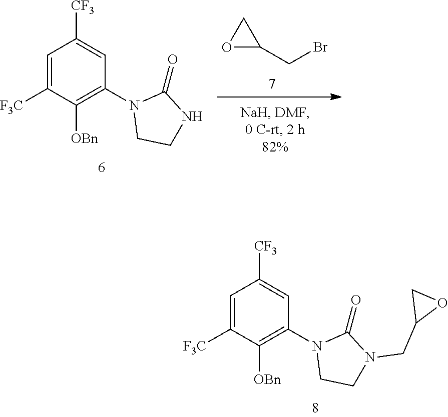 [SMILES: O=C1NCCN1c1cc(C(F)(F)F)cc(C(F)(F)F)c1OCc1ccccc1 ; BrCC1CO1 ; O=C1N(CC2CO2)CCN1c1cc(C(F)(F)F)cc(C(F)(F)F)c1OCc1ccccc1]

To a solution of compound 6 (405 mg, 1.0 mmol, 1.0 eq) in DMF (10 mL) was added NaH (80 mg, 2.0 mmol, 2.0 eq, 60%) at 0 °C. The reaction mixture was stirred for 30 mins at rt under N2 atmosphere. Compound 7 (206 mg, 1.5 mmol, 1.5 eq) was added to the mixture at 0 °C. The reaction mixture was stirred for 2 h at rt under N2 atmosphere. After completion of the reaction by TLC (R_{f}=0.4, PE/EtOAc=3/1), the mixture was extracted with EtOAc (50 mL×2). The combined organic layer was washed with brine, dried over Na₂SO₄ and filtered. The filtration was concentrated under reduced pressure. The residue was purified by column chromatography (100-200 silica gel, 40% EtOAc in PE as eluent) to afford compound 8 (380 mg, 82%) as colorless oil.

### Step 6: Synthesis of 1-(2-hydroxy-3,5-bis(trifluoromethyl)phenyl)-3-(oxiran-2-ylmethyl)imidazolidine-2-one

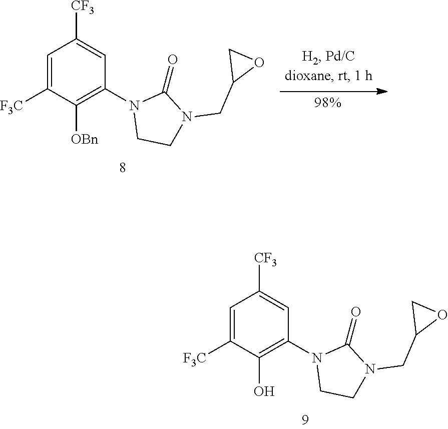 [SMILES: O=C1N(CC2CO2)CCN1c1cc(C(F)(F)F)cc(C(F)(F)F)c1OCc1ccccc1 ; O=C1N(CC2CO2)CCN1c1cc(C(F)(F)F)cc(C(F)(F)F)c1O]

To a solution of compound 8 (380 mg, 0.83 mmol, 1.0 eq) in dioxane (10 mL), was added Pd/C (114 mg, 30% wt) at rt under N2 atmosphere. The reaction was stirred for 1 h at rt. After completion of the reaction by TLC (R_{f}=0.2, PE/EtOAc=3/1), the mixture was concentrated under reduced pressure to afford compound 9 as pale-yellow oil (300 mg, 98%).

### Step 7: Synthesis of 2-(3-(oxiran-2-ylmethyl)-2-oxoimidazolidin-1-yl)-4,6-bis(trifluoromethyl)phenyl (4-fluorophenyl)(methyl-d₃)carbamate

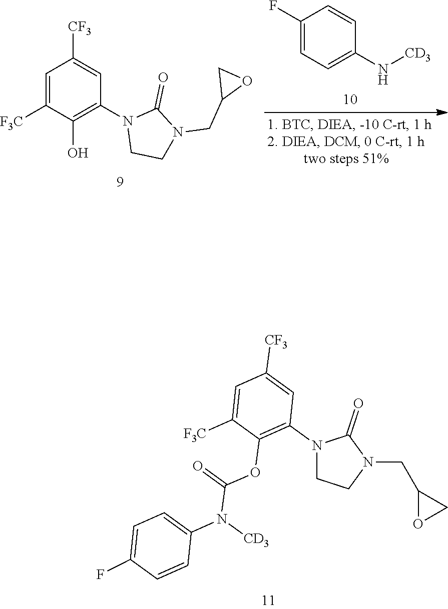 [SMILES: O=C1N(CC2CO2)CCN1c1cc(C(F)(F)F)cc(C(F)(F)F)c1O ; [2H]C([2H])([2H])Nc1ccc(F)cc1 ; [2H]C([2H])([2H])N(C(=O)Oc1c(N2CCN(CC3CO3)C2=O)cc(C(F)(F)F)cc1C(F)(F)F)c1ccc(F)cc1]

To a solution of compound 9 (330 mg, 0.89 mmol, 1.0 eq) in DCM (20 mL), was added DIEA (172 mg, 1.34 mmol, 1.5 eq) and triphosgene (132 mg, 0.45 mmol, 0.5 eq) in DCM (5 mL) at −10 °C. The reaction mixture was stirred for 1 h at rt under N2 atmosphere. The reaction solution was concentrated under reduced pressure to remove the solvent. Then DCM (5 mL) was added to above residue. The solution was used for next step.

To a solution of compound 10 (225 mg, 1.34 mmol, 1.5 eq) in DCM (20 mL) was added the above residue solution at 0 °C. The mixture was stirred for 1 h at rt under N2 atmosphere. After completion of the reaction by TLC (R_{f}=0.8, PE/EtOAc=3/1), HCl (1 M, 15 mL) was added. Then the mixture was extracted with DCM (40 mL×2). The combined organic layer was washed with brine, dried over Na₂SO₄ and filtered. The filtration was concentrated under reduced pressure. The residue was purified with FCC to give compound 11 (240 mg, 51%) as off-yellow oil.

### Step 8: Synthesis of 2-(3-(2-hydroxy-3-(methylamino)propyl)-2-oxoimidazolidin-1-yl)-4,6-bis(trifluoromethyl)phenyl (4-fluorophenyl)(methyl-d₃)carbamate

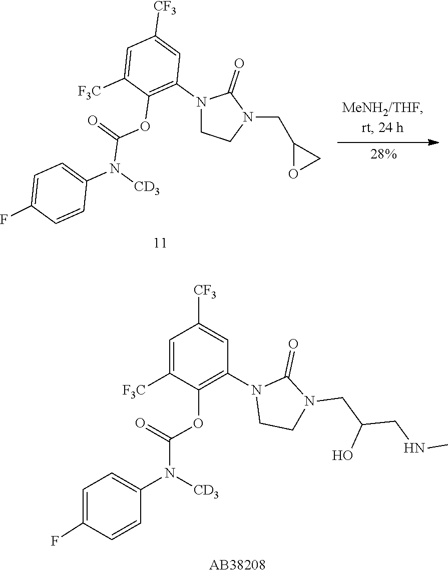 [SMILES: [2H]C([2H])([2H])N(C(=O)Oc1c(N2CCN(CC3CO3)C2=O)cc(C(F)(F)F)cc1C(F)(F)F)c1ccc(F)cc1 ; [2H]C([2H])([2H])N(C(=O)Oc1c(N2CCN(CC(O)CNC)C2=O)cc(C(F)(F)F)cc1C(F)(F)F)c1ccc(F)cc1]

To a solution of compound 11 (100 mg, 0.19 mmol, 1.0 eq) in THF (2 mL) was added MeNH₂/THF 2 mL at it. The reaction was stirred for 24 h at rt. After completion of the reaction by TLC (R_{f}=0.4, DCM/MeOH=10/1), the mixture was concentrated under reduced pressure. The residue was purified by Prep-HPLC to afford title compound (AB38208; 29.8 mg, 28%) as white solid.

¹H NMR (400 MHz, CD₃OD): δ 8.08 (s, 1H), 7.93-7.86 (m, 1H), 7.40 (s, 2H), 7.17 (t, 2H), 4.10 (br, 1H), 3.73-3.70 (m, 4H), 3.37-3.35 (m, 2H), 3.14-3.00 (m, 2H), 2.71-2.65 (m, 3H); LC-MS: 556.25 [M+H]⁺.

### Example 46

### 2-(3-(3-(dimethylamino)-2-hydroxypropyl)-2-oxoimidazolidin-1-yl)-4,6-bis(trifluoromethyl)phenyl (4-fluorophenyl)(methyl-d₃)carbamate

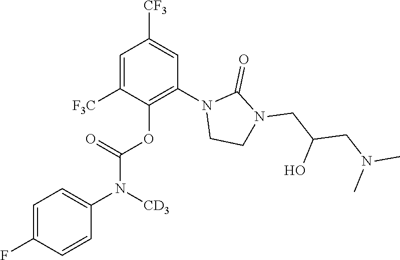 [SMILES: [2H]C([2H])([2H])N(C(=O)Oc1c(N2CCN(CC(O)CN(C)C)C2=O)cc(C(F)(F)F)cc1C(F)(F)F)c1ccc(F)cc1]

### Step 1: Synthesis of 1-(2-hydroxy-3,5-bis(trifluoromethyl)phenyl)imidazolidine-2-one

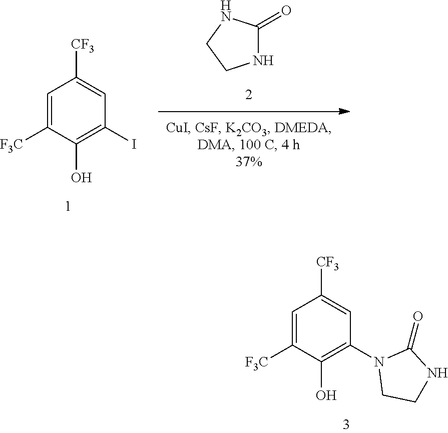 [SMILES: Oc1c(I)cc(C(F)(F)F)cc1C(F)(F)F ; O=C1NCCN1 ; O=C1NCCN1c1cc(C(F)(F)F)cc(C(F)(F)F)c1O]

To a solution of compound 1 (3.0 g, 8.45 mmol, 1.0 eq) and compound 2 (1.45 g, 16.9 mmol, 2.0 eq) in DMA (15 mL) were added CuI (803 mg, 4.23 mmol, 0.5 eq), DMEDA (744 mg, 8.45 mmol, 1.0 eq), K₂CO₃ (2.35 g, 16.9 mmol, 2.0 eq) and CsF (2.57 g, 16.9 mmol, 2.0 eq) at rt. The reaction mixture was stirred at 100 °C in an oil-bath for 4 h. After completion of the reaction by TLC (R_{f}=0.2, PE/EtOAc=3/1), water (50 mL) was added and adjust to pH 6 with 1N HCl. The mixture was extracted with EtOAc (100 mL×2). The combined organic layer was washed with brine, dried over Na₂SO₄ and filtered. The filtration was concentrated under reduced pressure. The residue was purified by column chromatography (100-200 silica gel, 40% EtOAc in PE as eluent) to afford compound 3 as pale green solid (1.0 g, 37%).

### Step 2: Synthesis of tert-butyl 3-(2-hydroxy-3,5-bis(trifluoromethyl)phenyl)-2-oxoimidazolidine-1-carboxylate

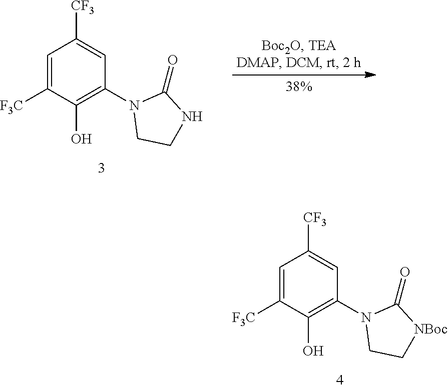 [SMILES: O=C1NCCN1c1cc(C(F)(F)F)cc(C(F)(F)F)c1O ; CC(C)(C)OC(=O)N1CCN(c2cc(C(F)(F)F)cc(C(F)(F)F)c2O)C1=O]

To a solution of compound 3 (1.0 g, 3.18 mmol, 1.0 eq) in DMF (10 mL) were added TEA (386 mg, 3.82 mmol, 1.2 eq), DMAP (40 mg, 0.318 mmol, 0.1 eq) and Boc₂O (728 mg, 3.50 mmol, 1.05 eq) at rt. The reaction was stirred for 2 h at rt. After completion of the reaction by TLC (R_{f}=0.4, PE/EtOAc=3/1). The mixture was extracted with EtOAc (50 mL×2). The combined organic layer was washed with brine, dried over Na₂SO₄ and filtered. The filtration was concentrated under reduced pressure. The residue was purified by column chromatography (100-200 silica gel, 35% EtOAc in PE as eluent) to afford compound 4 (500 mg, 38%) as off-white solid.

### Step 3: Synthesis of tert-butyl 3-(2-(benzyloxy)-3,5-bis(trifluoromethyl)phenyl)-2-oxoimidazolidine-1-carboxylate

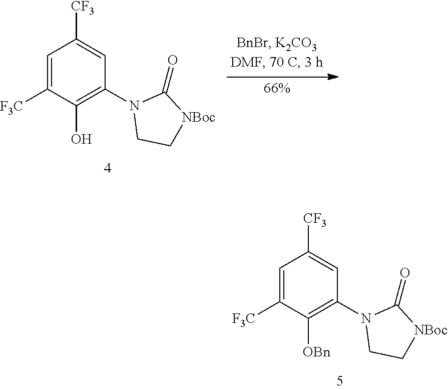 [SMILES: CC(C)(C)OC(=O)N1CCN(c2cc(C(F)(F)F)cc(C(F)(F)F)c2O)C1=O ; CC(C)(C)OC(=O)N1CCN(c2cc(C(F)(F)F)cc(C(F)(F)F)c2OCc2ccccc2)C1=O]

To a solution of compound 4 (566 mg, 1.37 mmol, 1.0 eq) in DCM (10 mL) were added K₂CO₃ (286 mg, 2.05 mmol, 1.5 eq) and BnBr (351 mg, 2.05 mmol, 1.5 eq) at rt. The reaction was stirred at 70 °C for 3 h. After completion of the reaction by TLC (R_{f}=0.9, PE/EtOAc=3/1), the reaction solution was poured into water (30 mL). The mixture was extracted with EtOAc (50 mL×2). The combined organic layer was washed with brine, dried over Na₂SO₄ and filtered. The filtration was concentrated under reduced pressure. The residue was purified by column chromatography (100-200 silica gel, 5% EtOAc in PE as eluent) to afford compound 5 as pale yellow solid (460 mg, 66%).

### Step 4: Synthesis of 1-(2-(benzyloxy)-3,5-bis(trifluoromethyl)phenyl)imidazolidine-2-one

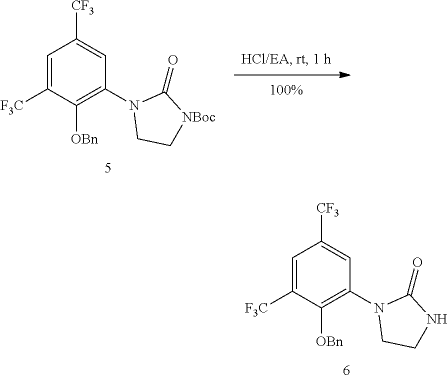 [SMILES: CC(C)(C)OC(=O)N1CCN(c2cc(C(F)(F)F)cc(C(F)(F)F)c2OCc2ccccc2)C1=O ; O=C1NCCN1c1cc(C(F)(F)F)cc(C(F)(F)F)c1OCc1ccccc1]

Compound 5 (460 mg, 0.91 mmol, 1.0 eq) was added into HCl/dioxane (4 M, 5 mL) at 0 °C. The reaction was stirred for 1 h at rt. After completion of the reaction by TLC (R_{f}=0.3, PE/EtOAc=3/1), the mixture was concentrated to give compound 6 (405 mg, 100%) as off-white solid.

### Step 5: Synthesis of 1-(2-(benzyloxy)-3,5-bis(trifluoromethyl)phenyl)-3-(oxiran-2-ylmethyl)imidazolidine-2-one

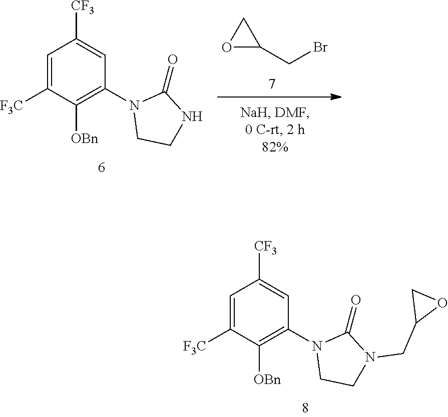 [SMILES: O=C1NCCN1c1cc(C(F)(F)F)cc(C(F)(F)F)c1OCc1ccccc1 ; BrCC1CO1 ; O=C1N(CC2CO2)CCN1c1cc(C(F)(F)F)cc(C(F)(F)F)c1OCc1ccccc1]

To a solution of compound 6 (405 mg, 1.0 mmol, 1.0 eq) in DMF (10 mL) was added NaH (80 mg, 2.0 mmol, 2.0 eq, 60%) at 0 °C. The reaction mixture was stirred for 30 mins at rt under N2 atmosphere. Compound 7 (206 mg, 1.5 mmol, 1.5 eq) was added to the mixture at 0 °C. The reaction mixture was stirred for 2 h at rt under N2 atmosphere. After completion of the reaction by TLC (R_{f}=0.4, PE/EtOAc=3/1), the mixture was extracted with EtOAc (50 mL×2). The combined organic layer was washed with brine, dried over Na₂SO₄ and filtered. The filtration was concentrated under reduced pressure. The residue was purified by column chromatography (100-200 silica gel, 40% EtOAc in PE as eluent) to afford compound 8 (380 mg, 82%) as colorless oil.

### Step 6: Synthesis of 1-(2-hydroxy-3,5-bis(trifluoromethyl)phenyl)-3-(oxirann-2-ylmethyl)imidazolidin-2-one

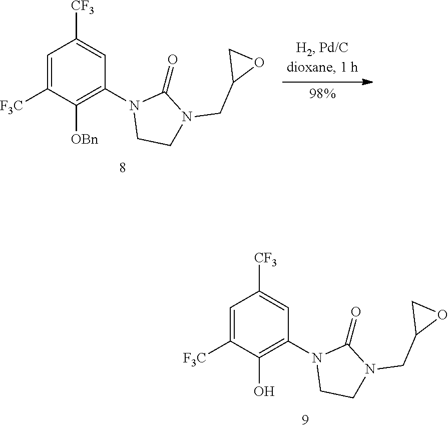 [SMILES: O=C1N(CC2CO2)CCN1c1cc(C(F)(F)F)cc(C(F)(F)F)c1OCc1ccccc1 ; O=C1N(CC2CO2)CCN1c1cc(C(F)(F)F)cc(C(F)(F)F)c1O]

To a solution of compound 8 (380 mg, 0.83 mmol, 1.0 eq) in dioxane (10 mL), was added Pd/C (114 mg, 30% wt) at rt under N2 atmosphere. The reaction was stirred for 1 h at rt. After completion of the reaction by TLC (R_{f}=0.2, PE/EtOAc=3/1), the mixture was concentrated under reduced pressure to afford compound 9 as pale-yellow oil (300 mg, 98%).

### Step 7: Synthesis of 2-(3-(oxiran-2-ylmethyl)-2-oxoimidazolidin-1-yl)-4,6-bis(trifluoromethyl)phenyl (4-fluorophenyl)(methyl-d₃)carbamate

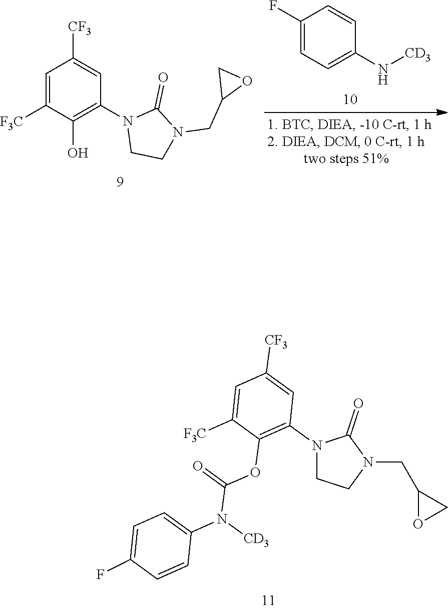 [SMILES: O=C1N(CC2CO2)CCN1c1cc(C(F)(F)F)cc(C(F)(F)F)c1O ; [2H]C([2H])([2H])Nc1ccc(F)cc1 ; [2H]C([2H])([2H])N(C(=O)Oc1c(N2CCN(CC3CO3)C2=O)cc(C(F)(F)F)cc1C(F)(F)F)c1ccc(F)cc1]

To a solution of compound 9 (330 mg, 0.89 mmol, 1.0 eq) in DCM (20 mL), was added DIEA (172 mg, 1.34 mmol, 1.5 eq) and triphosgene (132 mg, 0.45 mmol, 0.5 eq) in DCM (5 mL) at −10 °C. The reaction mixture was stirred for 1 h at rt under N2 atmosphere. The reaction solution was concentrated under reduced pressure to remove the solvent. Then DCM (5 mL) was added to above residue. The solution was used for next step.

To a solution of compound 10 (225 mg, 1.34 mmol, 1.5 eq) in DCM (20 mL) was added the above residue solution at 0 °C. The mixture was stirred for 1 h at rt under N2 atmosphere. After completion of the reaction by TLC (R_{f}=0.8, PE/EtOAc=3/1), HCl (1 M, 15 mL) was added. Then the mixture was extracted with DCM (40 mL×2). The combined organic layer was washed with brine, dried over Na₂SO₄ and filtered. The filtration was concentrated under reduced pressure. The residue was purified with FCC to give compound 11 (240 mg, 51%) as off-yellow oil.

### Step 8: Synthesis of 2-(3-(3-(dimethylamino)-2-hydroxypropyl)-2-oxoimidazolidin-1-yl)-4,6-bis(trifluoromethyl)phenyl (4-fluorophenyl)(methyl-d₃)carbamate

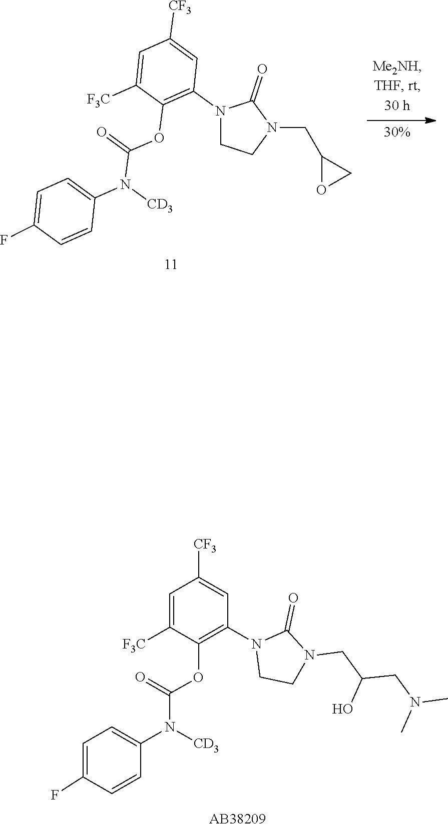 [SMILES: [2H]C([2H])([2H])N(C(=O)Oc1c(N2CCN(CC3CO3)C2=O)cc(C(F)(F)F)cc1C(F)(F)F)c1ccc(F)cc1 ; [2H]C([2H])([2H])N(C(=O)Oc1c(N2CCN(CC(O)CN(C)C)C2=O)cc(C(F)(F)F)cc1C(F)(F)F)c1ccc(F)cc1]

To a solution of compound 11 (100 mg, 0.19 mmol, 1.0 eq) in THF (2 mL) was added Me₂NH (2 mL, 2M in THF) at rt. The reaction was stirred for 30 h at it. After completion of the reaction by TLC (R_{f}=0.4, DCM/MeOH=10/1), the mixture was concentrated under reduced pressure. The residue was purified by Prep-HPLC to afford title compound (AB38209; 33.4 mg, 30%) as white solid.

¹H NMR (400 MHz, CD₃OD) δ8.09-8.08 (m, 1H), 7.92-7.85 (m, 1H), 7.39 (s, 2H), 7.16 (t, 2H), 4.21 (s, 1H) 3.85-3.68 (m, 4H), 3.39-3.37 (m, 2H), 3.19-3.14 (m, 2H), 2.90-2.85 (m, 6H); LC-MS: 570.30 [M+H]⁺.

### Example 47

### 2-(3-(2-hydroxy-3-(methylamino)propyl)-2-oxoimidazolidin-1-yl)-4,6-bis(trifluoromethyl)phenyl (3-chloro-2,4-difluorophenyl)(methyl)carbamate

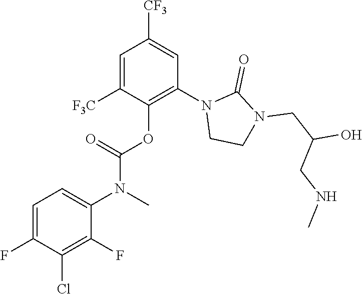 [SMILES: CNCC(O)CN1CCN(c2cc(C(F)(F)F)cc(C(F)(F)F)c2OC(=O)N(C)c2ccc(F)c(Cl)c2F)C1=O]

### Step 1:(3-chloro-2,4-difluorophenyl)(methyl)carbamic chloride

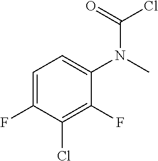

Same experimental procedure as step 1 of example 7. ESIMS: m/z 240.09 [(M+H)⁺].

### Step 2: Synthesis of 2-iodo-4,6-bis(trifluoromethyl)phenyl (3-chloro-2,4-difluorophenyl)(methyl)carbamate

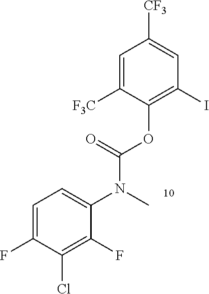 [SMILES: CN(C(=O)Oc1c(I)cc(C(F)(F)F)cc1C(F)(F)F)c1ccc(F)c(Cl)c1F]

2-iodo-4,6-bis(trifluoromethyl)phenol (570 mg, 1.60 mmol) and (3-chloro-2,4-difluorophenyl)(methyl)carbamic chloride (300 mg, 0.84 mmol) were dissolved into anhydrous pyridine (5 mL). This solution was stirred at 90 °C for 4 hours. The reaction was cooled to RT and concentrated down. The residual solid was partitioned between EtOAc and 1N aqueous HCl. The aqueous phase was separated and extracted with EtOAc twice. The combined organic extracts were washed with brine, dried over anhydrous sodium sulfate and concentrated. The crude product was purified by column chromatography on silica gel using a gradient solvent system of 0 to 20% of ethyl acetate in hexanes to afford the titled compound as a yellow oil (227 mg, 48%) ESIMS: m/z 560.21 [(M+H)⁺].

### Step 3: Synthesis of 2-(2-oxoimidazolidin-1-yl)-4,6-bis(trifluoromethyl)phenyl (3-chloro-2,4-difluorophenyl)(methyl)carbamate

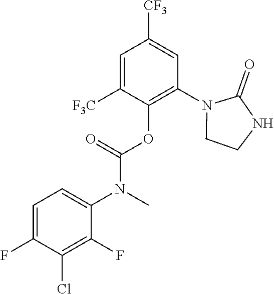 [SMILES: CN(C(=O)Oc1c(N2CCNC2=O)cc(C(F)(F)F)cc1C(F)(F)F)c1ccc(F)c(Cl)c1F]

2-iodo-4,6-bis(trifluoromethyl)phenyl (3-chloro-2,4-difluorophenyl)(methyl)carbamate (0.2269 mmol, 126 mg), 2-imidazolidone (0.4548 mmol, 39 mg), copper (I) iodide (0.11135 mmol, 22 mg), cesium fluoride (0.4538 mmol, 69 mg), N,N′-dimethylethylenediamine (0.2269 mmol, 25 uL) and anhydrous powered potassium carbonate (0.4538 mmol, 63 mg) were added to degassed anhydrous 1,4-dioxane (5.0 mL) under nitrogen. The resulting suspension was stirred at 90 °C overnight. The reaction was cooled to room temperature, filtered and the filtrate was concentrated down to yield a semi-solid. The crude product was purified by column chromatography on silica gel using a gradient solvent system of 0 to 100% of ethyl acetate in hexanes to afford the titled compound as a yellow oil (6 mg, 5%).

¹H NMR (400 MHz, CDCl₃) δ 7.83 (m, 2H), 7.19 (m, 2H), 3.88 (m, 2H), 3.64 (t, J=12 Hz, 2H), 3.4 (d, J=56 Hz, 3H), 3.21 (s, 1H); ESIMS: m/z 518.3 [(M+H)⁺].

### Step 4: Synthesis of 2-(3-(oxiran-2-ylmethyl)-2-oxoimidazolidin-1-yl)-4,6-bis(trifluoromethyl)phenyl (3-chloro-2,4-difluorophenyl)(methyl)carbamate

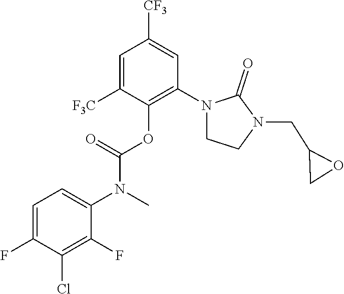 [SMILES: CN(C(=O)Oc1c(N2CCN(CC3CO3)C2=O)cc(C(F)(F)F)cc1C(F)(F)F)c1ccc(F)c(Cl)c1F]

To a stirred solution of 2-(2-oxoimidazolidin-1-yl)-4,6-bis(trifluoromethyl)phenyl (3-chloro-2,4-fluorophenyl)(methyl)carbamate (0.104 g, 0.0002 mol) in DMF (8 mL) was purged with N2 for 10 minutes. To the above solution added K₃PO₄ (0.27 g, 0.00128 mol) at RT and continued purging for 10 min. After 30 min of stirring at RT, reaction mixture was cooled to 0 °C and to the same added epibromohydrin (0.12 mL, 0.00128 mol) and KI (0.13 g, 0.0008 mol). The reaction mixture was stirred at RT for 20 h. After completion of the reaction confirmed by TLC (R_{f} 0.3, 60% EtOAc in Hexane) the reaction mixture was diluted with cold water (150 mL) followed by extraction with ethyl acetate (2*100 mL). The organic fractions separated were combined, dried over anhydrous Na₂SO₄, filtered and concentrated under reduced pressure to afford crude. The crude residue was purified by preparative TLC (solid phase: Merck, 20×20 cm, silicagel 60 GF₂₅₄, 1 mm, PLC glass plate, 50% EtOAc in Hexane as eluent) to afford the desired title compound as sticky oil (40 mg, 38%).

ESIMS: m/z 574.10 [(M+H)⁺].

### Step 5: Synthesis of 2-(3-(2-hydroxy-3-(methylamino)propyl)-2-oxoimidazolidin-1-yl)-4,6-bis(trifluoromethyl)phenyl (3-chloro-2,4-difluorophenyl)(methyl)carbamate

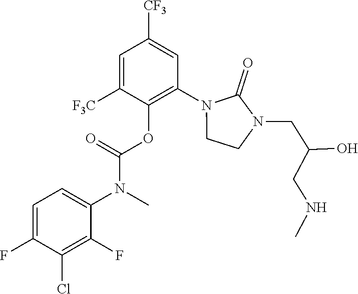 [SMILES: CNCC(O)CN1CCN(c2cc(C(F)(F)F)cc(C(F)(F)F)c2OC(=O)N(C)c2ccc(F)c(Cl)c2F)C1=O]

To a solution of 2,4-bis(trifluoromethyl)-6-(3-((oxiran-2-yl)methyl)-2-oxoimidazolidin-1-yl)phenyl 4-fluorophenylmethylcarbamate (0.020 g, 0.035 mmol) in 3 mL anhydrous methanol at 0 °C. 2M Methylamine (2 mL, 4 mmol) was added. The reaction was stirred for 20 h at room temperature. The solvent was removed under reduced pressure. The oil was dissolved in dichloromethane and triturated with hexanes. The solvent was removed under reduced pressure to afford title compound as semi solid (15 mg, 48% yield).

¹H NMR (400 MHz, MeOD) δ 8.1 (d, J=20 Hz, 1H), 7.91 (d, J=24, Hz, 1H), 7.52 (m, 1H), 7.24 (m, 1H), 3.98 (m, 1H),), 3.74 (m, 3H), 3.48 (s, 1H), 3.35 (m, 4H), 3.26 (m, 2H), 2.69 (m, 2H), 2.45 (d, J=12 Hz 3H); ESIMS: m/z 605.22.24 [(M+H)⁺].

### Example 48

### 2-(3-(2-hydroxy-3-(methylamino)propyl)-2-oxoimidazolidin-1-yl)-4,6-bis(trifluoromethyl)phenyl (4-fluorophenyl)(methyl)carbamate

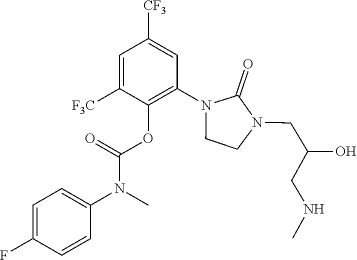 [SMILES: CNCC(O)CN1CCN(c2cc(C(F)(F)F)cc(C(F)(F)F)c2OC(=O)N(C)c2ccc(F)cc2)C1=O]

### Step 1: Synthesis of 2-iodo-4,6-bis(trifluoromethyl)phenol

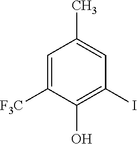

A solution of 2,4-bis(trifluoromethyl)phenol (1.0 g, 0.0043 mol) in THF:H₂O (3:1, 20:6 mL) was cooled 0 °C in an ice bath with stirring. After 15 minutes, was added 12 (1.43 g, 0.0056 mol) followed by Na₂CO₃ (0.68 g, 0.0064 mol) under inert atmosphere with stirring. Allowed the reaction mixture to stir at ambient temperature for 24 h. After completion of the reaction (TLC: R_{f} −0.4, 30% EtOAc in Hexane followed by 30% DCM in Hexane), the reaction mixture was cooled to 0 °C and then quenched with aq. sodium metabisulfite solution followed by extraction with EtOAc (2×100 mL). The organic layer collected dried over anhydrous Na₂SO₄, filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 2: Synthesis of (4-fluorophenyl)(methyl)carbamic chloride

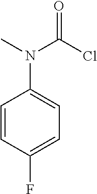

To a cold (0 °C) solution of triphosgene (1.6 g, 0.0128 mol) in DCM (40 mL) added a solution of N-methyl-4-fluoro aniline (1.89 g, 0.0064 mol) and pyridine (2.52 g, 2.6 mL, 0.032 mol) dropwise for over a period of 10 min. After that continued stirring at RT for 16 h. Progress of the reaction was monitored by TLC (R_{f} −0.7, 10/a EtOAc in Hexane (×4)). After completion of the reaction, quenched the reaction mixture with 1M aq·HCl (50 mL) and then extracted with DCM (2×50 mL). The DCM layer separated was dried over anhydrous Na₂SO₄, filtered, and concentrated under reduced pressure to afford the title compound as green solid (1.8 g, 75%).

### Step 3: Synthesis of 2-iodo-4,6-bis(trifluoromethyl)phenyl (4-fluorophenyl)(methyl)carbamate

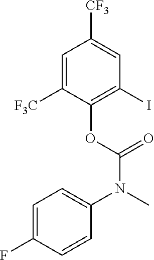 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80 °C for 4 hours. After completion of the reaction was confirmed by TLC (R_{f} −0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off-white solid (0.66 g, 93%).

### Step 4: Synthesis of 2-(2-oxoimidazolidin-1-yl)-4,6-bis(trifluoromethyl)phenyl (4-fluorophenyl)methyl) carbamate

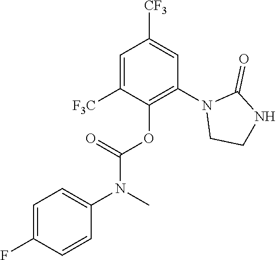 [SMILES: CN(C(=O)Oc1c(N2CCNC2=O)cc(C(F)(F)F)cc1C(F)(F)F)c1ccc(F)cc1]

2-iodo-4,6-bis(trifluoromethyl)phenyl (4-fluorophenyl)(methyl)carbamate (0.2 g, 0.00039 mol), 2-imidazilidinone (0.064 g, 0.0007 mol), copper (I)-iodide (0.037 g, 0.00019 mol), N,N′-dimethylethylenediamine (0.07 mL, 0.00039 mol), caesium fluoride (0.12 g, 0.00079 mol) and potassium carbonate (0.1 g, 0.00072 mol) were suspended in 1,4-dioxane priorly purged with N2 for 30 minutes (16 mL). The reaction mixture was heated at 90 °C in an oil-bath for 24 h. After completion of the reaction by TLC (R_{f} 0.4, 5% MeOH in DCM) the reaction mixture was concentrated under reduced pressure. The residue obtained was purified by column chromatography (100-200 silica gel, 0-3% MeOH in DCM as eluent) to afford the title compound as off-white solid (50 mg). The solid was re-purified by preparative TLC (solid phase: merck, 20×20 cm, silicagel 60 GF₂₅₄, 1 mm, PLC glass plate, 2% MeOH in DCM as eluent) to afford white solid (28 mg, 15%).

### Step 5: Synthesis of 2-(3-(oxiran-2-ylmethyl)-2-oxoimidazolidin-1-yl)-4,6-bis(trifluoromethyl)phenyl (4-fluorophenyl)(methyl)carbamate

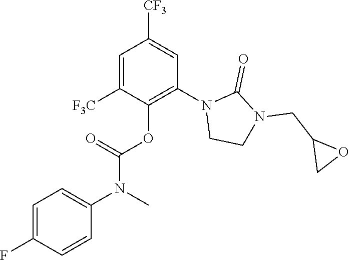 [SMILES: CN(C(=O)Oc1c(N2CCN(CC3CO3)C2=O)cc(C(F)(F)F)cc1C(F)(F)F)c1ccc(F)cc1]

To a stirred solution of 2-(2-oxoimidazolidin-1-yl)-4,6-bis(trifluoromethyl)phenyl (4-fluorophenyl)(methyl)carbamate (0.1 g, 0.0002 mol) in DMF (8 mL) was purged with N2 for 10 minutes. To the above solution added K₃PO₄ (0.27 g, 0.00128 mol) at RT and continued purging for 10 min. After 30 min of stirring at RT, reaction mixture was cooled to 0 °C and to the same added epibromohydrin (0.12 mL, 0.00128 mol) and KI (0.13 g, 0.0008 mol). The reaction mixture was stirred at RT for 20 h. After completion of the reaction confirmed by TLC (R_{f} 0.3, 60% EtOAc in Hexane) the reaction mixture was diluted with cold water (150 mL) followed by extraction with ethyl acetate (2*100 mL). The organic fractions separated were combined, dried over anhydrous Na₂SO₄, filtered and concentrated under reduced pressure to afford crude. The crude residue was purified by preparative TLC (solid phase: Merck, 20×20 cm, silicagel 60 GF₂₅₄, 1 mm, PLC glass plate, 50% EtOAc in Hexane as eluent) to afford the desired title compound as sticky oil (40 mg, 38%).

### Step 6: Synthesis of 2-(3-(2-hydroxy-3-(methylamino)propyl)-2-oxoimidazolidin-1-yl)-4,6-bis(trifluoromethyl)phenyl (4-fluorophenyl)(methyl)carbamate

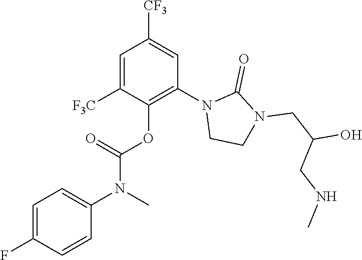 [SMILES: CNCC(O)CN1CCN(c2cc(C(F)(F)F)cc(C(F)(F)F)c2OC(=O)N(C)c2ccc(F)cc2)C1=O]

To a solution of 2-(3-(oxiran-2-ylmethyl)-2-oxoimidazolidin-1-yl)-4,6-bis(trifluoromethyl)phenyl (4-fluorophenyl(methyl)carbamate (0.025 g, 0.047 mmol) in 3 mL anhydrous methanol at 0 °C. 2M Methylamine (2 mL, 4 mmol) was added. The reaction was stirred for 20 h at room temperature. The solvent was removed under reduced pressure. The oil was dissolved in dichloromethane and triturated with hexanes. The solvent was removed under reduced pressure to afford title compound as semi solid (26 mg, 100% yield).

¹H NMR (400 MHz, MeOD) δ 8.09 (bs, 1H), 7.8 (m, 1H), 7.42 (bs, 2H), 7.17 (m, 2H), 3.98 (bs, 1H),), 3.71 (m, 3H), 3.51 (bs, 1 h), 3.34 (m, 4H), 3.36 (m, 2H), 2.66 (m, 2H), 2.43 (m, 3H); ESIMS: m/z 553.24 [(M+H)⁺].

### Example 49

### (S)-2-(3-(3-(dimethylamino)-2-hydroxypropyl)-2-oxoimidazolidin-1-yl)-4,6-bis(trifluoromethyl)phenyl (4-fluorophenyl)(methyl-d₃)carbamate

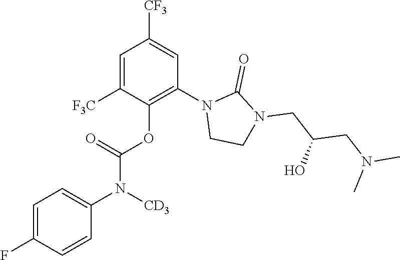 [SMILES: [2H]C([2H])([2H])N(C(=O)Oc1c(N2CCN(C[C@@H](O)CN(C)C)C2=O)cc(C(F)(F)F)cc1C(F)(F)F)c1ccc(F)cc1]

### Step 1: Synthesis of 1-(2-hydroxy-3,5-bis(trifluoromethyl)phenyl)imidazolidine-2-one

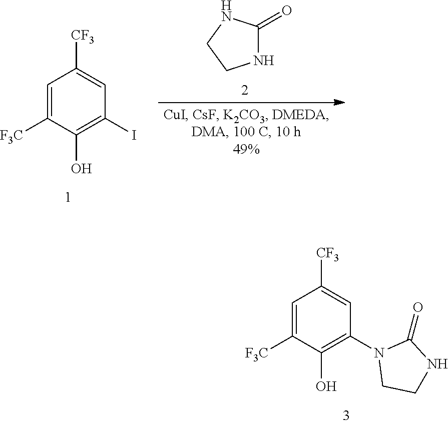 [SMILES: Oc1c(I)cc(C(F)(F)F)cc1C(F)(F)F ; O=C1NCCN1 ; O=C1NCCN1c1cc(C(F)(F)F)cc(C(F)(F)F)c1O]

To a solution of compound 1 (30.0 g, 84.5 mmol, 1.0 eq) in DMA (200 mL) were added compound 2 (14.5 g, 169 mmol, 2.0 eq), CuI (8.03 g, 42.3 mmol, 0.5 eq), DMEDA (7.44 g, 84.5 mmol, 1.0 eq), K₂CO₃ (23.5 g, 169 mmol, 2.0 eq) and CsF (25.7 g, 169 mmol, 2.0 eq) at 0 °C. The reaction mixture was stirred at 100 °C for 10 h. After completion of the reaction by TLC, the reaction solution was poured into water (500 mL). The mixture was extracted with ethyl acetate (500 mL×2). The combined organic layer was washed with brine, dried over Na₂SO₄ and filtered. The filtration was concentrated under reduced pressure. The residue was purified by column chromatography (100-200 silica gel, 40% EA in PE as eluent) to afford compound 3 (13.0 g, 49%) as yellow solid.

TLC: PE/EA=3/1

R_{f} (Compound 1)=0.4

R_{f} (Compound 3)=0.3

LC-MS: 315.0 [M+1]⁺.

### Step 2: Synthesis of tert-butyl 3-(2-hydroxy-3,5-bis(trifluoromethyl)phenyl)-2-oxoimidazolidine-1-carboxylate

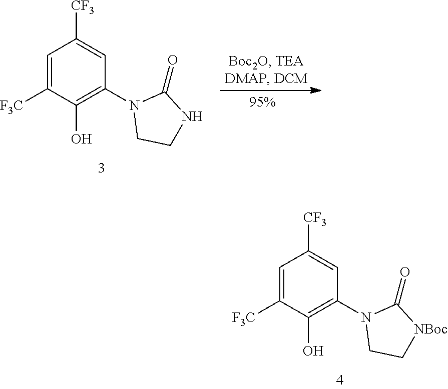 [SMILES: O=C1NCCN1c1cc(C(F)(F)F)cc(C(F)(F)F)c1O ; CC(C)(C)OC(=O)N1CCN(c2cc(C(F)(F)F)cc(C(F)(F)F)c2O)C1=O]

To a solution of compound 3 (8.0 g, 25.4 mmol, 1.0 eq) in DCM (100 mL) were added TEA (3.08 g, 30.5 mmol, 1.2 eq), Boc₂O (5.83 g, 26.7 mmol, 1.05 eq) and DMAP (310 mg, 2.54 mmol, 0.1 eq) at rt. The reaction mixture was stirred for 2 h at rt. After completion of the reaction by, the reaction solution was poured into water (300 mL). The solution was extracted with ethyl acetate (500 mL×2). The combined organic layer was washed with brine, dried over Na₂SO₄ and filtered. The filtrate was concentrated under reduced pressure to afford compound 4 (10 g, 95%) as brown solid.

TLC: DCM/MeOH=20/1

R_{f} (Compound 3)=0.6

R_{f} (Compound 4)=0.5

LC-MS: 412.9 [M−1]⁻.

### Step 3: Synthesis of tert-butyl 3-(2-hydroxy-3,5-bis(trifluoromethyl)phenyl)-2-oxoimidazolidine-1-carboxylate

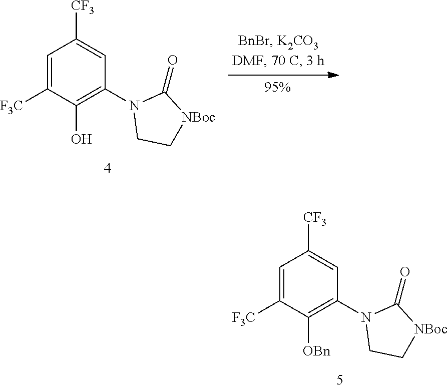 [SMILES: CC(C)(C)OC(=O)N1CCN(c2cc(C(F)(F)F)cc(C(F)(F)F)c2O)C1=O ; CC(C)(C)OC(=O)N1CCN(c2cc(C(F)(F)F)cc(C(F)(F)F)c2OCc2ccccc2)C1=O]

To a solution of compound 4 (10.0 g, 24.1 mmol, 1.0 eq) in DMF (100 mL) were added K₂CO₃ (5.03 g, 36.2 mmol, 1.5 eq) and BnBr (6.19 g, 36.2 mmol, 1.5 eq) at rt. The reaction mixture was stirred at 70 °C for 3 h. After completion of the reaction by TLC, the reaction solution was poured into water (300 mL). The solution was extracted with ethyl acetate (500 mL×2). The combined organic layer was washed with brine, dried over Na₂SO₄ and filtered. The filtrate was concentrated under reduced pressure. The residue was purified by column chromatography (100-200 silica gel, 5% EA in PE as eluent) to afford compound 5 (5.6 g, 46%) as yellow solid.

TLC: PE/EA=5/1

R_{f} (Compound 4)=0.1

R_{f} (Compound 5)=0.8

LC-MS: 449.0 [M+1-56]~; 405.0 [M+1-100]⁺.

### Step 4: Synthesis of 1-(2-(benzyloxy)-3,5-bis(trifluoromethyl)phenyl)imidazolidine-2-one

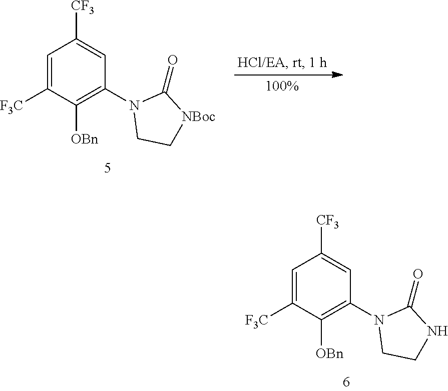 [SMILES: CC(C)(C)OC(=O)N1CCN(c2cc(C(F)(F)F)cc(C(F)(F)F)c2OCc2ccccc2)C1=O ; O=C1NCCN1c1cc(C(F)(F)F)cc(C(F)(F)F)c1OCc1ccccc1]

To a solution of compound 5 (8.2 g, 16.3 mmol, 1.0 eq) in ethyl acetate (10 mL) was added HCl/ethyl acetate (50 mL) at 0 °C. The reaction mixture was stirred at rt for 1 h. After completion of the reaction by, the reaction solution was concentrated under reduced pressure to afford compound 6 (6.7 g, 100%) as yellow solid.

TLC: PE/EA=5/1

R_{f} (Compound 5)=0.8

R_{f} (Compound 6)=0.2

LC-MS: 405.0 [M+1]⁺.

### Step 5: Synthesis of (R)-1-(2-(benzyloxy)-3,5-bis(trifluoromethyl)phenyl)-3-(oxiran-2-ylmethyl)imidazolidine-2-one

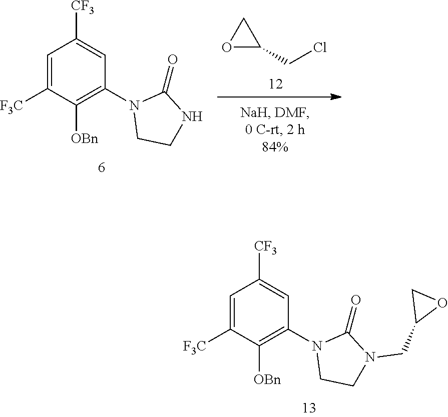 [SMILES: O=C1NCCN1c1cc(C(F)(F)F)cc(C(F)(F)F)c1OCc1ccccc1 ; ClC[C@H]1CO1 ; O=C1N(C[C@@H]2CO2)CCN1c1cc(C(F)(F)F)cc(C(F)(F)F)c1OCc1ccccc1]

To a solution of compound 6 (520 mg, 1.28 mmol, 1.0 eq) in DMF (5 mL) was added 60% NaH (128 mg, 3.20 mmol, 2.5 eq) at 0 °C. The reaction mixture was stirred at rt for 30 mins. The compound 15 (177 mg, 1.92 mmol, 1.5 eq) was added. The reaction mixture was stirred at rt for 1.5 h. After completion of the reaction by TLC, the reaction solution was poured into water (100 mL). The mixture was extracted with ethyl acetate (150 mL×2). The combined organic layer was washed with brine, dried over Na₂SO₄ and filtered. The filtrate was concentrated under reduced pressure. The residue was purified by column chromatography (100-200 silica gel, 45% EA in PE as eluent) to afford compound 13 (500 mg, 84%) as yellow oil.

TLC: PE/EA=3/1

R_{f} (Compound 6)=0.4

R_{f} (Compound 13)=0.5

LC-MS: 461.1 [M+1]⁺

### Step 6: Synthesis of (R)-1-(2-hydroxy-3,5-bis(trifluoromethyl)phenyl)-3-(oxiran-2-ylmethyl)imidazolidine-2-one

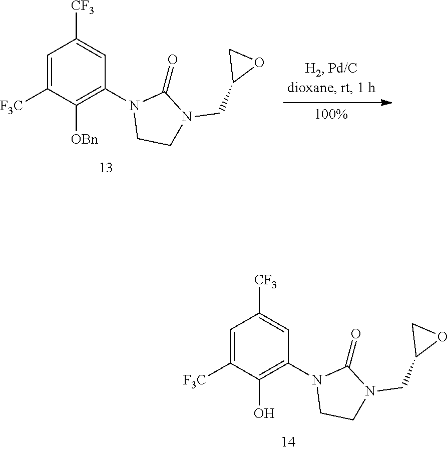 [SMILES: O=C1N(C[C@@H]2CO2)CCN1c1cc(C(F)(F)F)cc(C(F)(F)F)c1OCc1ccccc1 ; O=C1N(C[C@@H]2CO2)CCN1c1cc(C(F)(F)F)cc(C(F)(F)F)c1O]

To a solution of compound 13 (500 mg, 1.08 mmol, 1.0 eq) in dioxane (5 mL) was added Pd/C (100 mg, 20% wt) at rt. The reaction mixture was stirred for 1 h at rt. After completion of the reaction by TLC, the mixture was filtered with celite. The filtrate was concentrated under reduced pressure to afford compound 14 (420 mg, 100%) as colorless oil.

TLC: PE/EA=3/1

R_{f} (Compound 13)=0.5

R_{f} (Compound 14)=0.4

LC-MS: 371.1 [M+1]⁺.

### Step 7: Synthesis of (R)-2-(3-(oxiran-2-ylmethyl)-2-oxoimidazolidin-1-yl)-4,6-bis(trifluoromethyl)phenyl (4-fluorophenyl)(methyl-d₃)carbamate

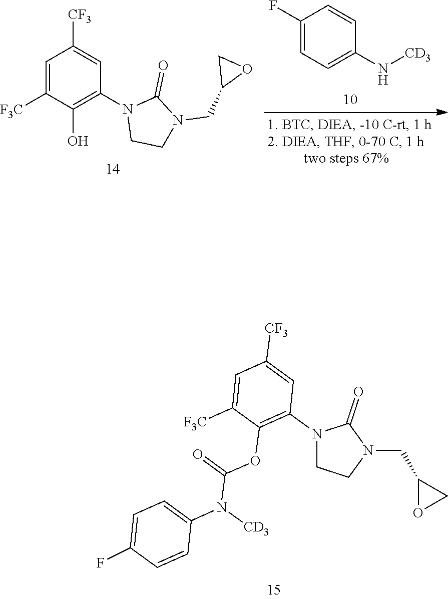 [SMILES: O=C1N(C[C@@H]2CO2)CCN1c1cc(C(F)(F)F)cc(C(F)(F)F)c1O ; [2H]C([2H])([2H])Nc1ccc(F)cc1 ; [2H]C([2H])([2H])N(C(=O)Oc1c(N2CCN(C[C@@H]3CO3)C2=O)cc(C(F)(F)F)cc1C(F)(F)F)c1ccc(F)cc1]

To a solution of compound 14 (420 mg, 1.13 mmol, 1.0 eq) in DCM (20 mL) were added DIEA (146 mg, 1.13 mmol, 1.0 eq), triphosgene (335 mg, 1.13 mmol, 1.0 eq) at −10 °C. The reaction mixture was stirred at rt for 1 h. The reaction solution was concentrated under reduced pressure to remove the solvent. Then DCM (10 mL) was added to above residue. The solution was used for next step. To a solution of compound 10 (218 mg, 1.70 mmol, 1.5 eq) in DCM (10 mL) was added the above residue solution at 0 °C. The mixture was stirred at rt for 1 h. After completion of the reaction by TLC, the reaction solution was poured into water (100 mL). The mixture was extracted with DCM (150 mL×2). The combined organic layer was washed with brine, dried over Na₂SO₄ and filtered. The filtrate was concentrated under reduced pressure. The residue was purified by column chromatography (100-200 silica gel, 65% EA in PE as eluent) to afford compound 15 (400 mg, 67%) as yellow oil.

TLC: PE/EA=3/1

R_{f} (Compound 13)=0.4

R_{f} (Compound 14)=0.3

LC-MS: 525.2 [M+1]⁺.

### Step 8: Synthesis of (S)-2-(3-(3-(dimethylamino)-2-hydroxypropyl)-2-oxoimidazolidin-1-yl)-4,6-bis(trifluoromethyl)phenyl (4-fluorophenyl)(methyl-d₃)carbamate

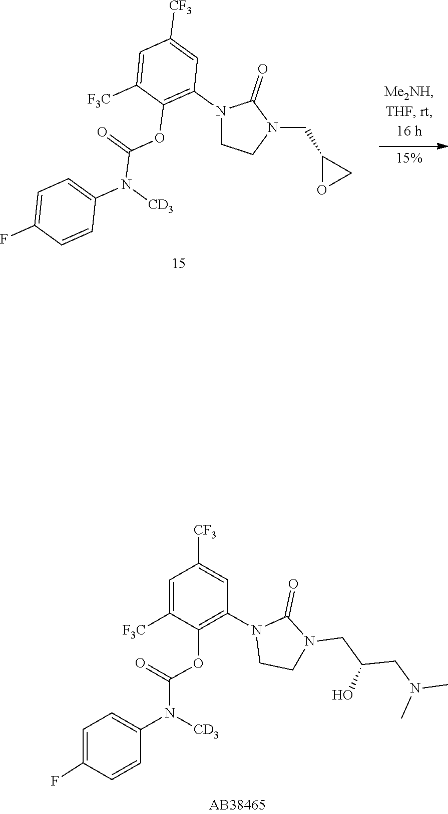 [SMILES: [2H]C([2H])([2H])N(C(=O)Oc1c(N2CCN(C[C@@H]3CO3)C2=O)cc(C(F)(F)F)cc1C(F)(F)F)c1ccc(F)cc1 ; [2H]C([2H])([2H])N(C(=O)Oc1c(N2CCN(C[C@@H](O)CN(C)C)C2=O)cc(C(F)(F)F)cc1C(F)(F)F)c1ccc(F)cc1]

To a solution of compound 15 (150 mg, 0.28 mmol, 1.0 eq) in THF (2 mL) at rt were added Me₂NH (0.85 mL 1.0 M/L in THF, 3.0 eq). The mixture was stirred for 16 h at rt. After completion of the reaction by TLC, the mixture was concentrated and the residue was purified by column chromatography (100-200 silica gel, 9.5% MeOH in DCM as eluent) to afford title compound (AB38465, 25.0 mg, 15%) as off-white solid.

TLC: DCM/MeOH=10/1

R_{f} (Compound 15)=0.5

R_{f} (AB38465)=0.4

¹H NMR (400 MHz, CD₃OD) δ 8.08 (br, 1H), 7.83-7.90 (m, 1H), 7.40 (s, 2H), 7.15 (t, J=8.4 Hz, 2H), 3.98-3.71 (m, 5H), 3.38-3.33 (m, 1H), 3.27-3.21 (m, 1H), 2.49 (br s, 2H), 2.37-2.33 (m, 6H); LC-MS: 570.1 [M+H]⁺.

### Example 50

### (R)-2-(3-(3-(dimethylamino)-2-hydroxypropyl)-2-oxoimidazolidin-1-yl)-4,6-bis(trifluoromethyl)phenyl (4-fluorophenyl)(methyl-d3)carbamate

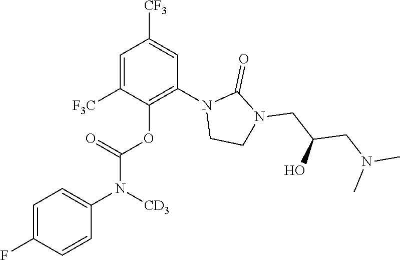 [SMILES: [2H]C([2H])([2H])N(C(=O)Oc1c(N2CCN(C[C@H](O)CN(C)C)C2=O)cc(C(F)(F)F)cc1C(F)(F)F)c1ccc(F)cc1]

### Step 1: Synthesis of 1-(2-hydroxy-3,5-bis(trifluormethyl)phenyl)imidazolidine-2-one

To a solution of compound 1 (30.0 g, 84.5 mmol, 1.0 eq) in DMA (200 mL) were added compound 2 (14.5 g, 169 mmol, 2.0 eq), CuI (8.03 g, 42.3 mmol, 0.5 eq), DMEDA (7.44 g, 84.5 mmol, 1.0 eq), K₂CO₃ (23.5 g, 169 mmol, 2.0 eq) and CsF (25.7 g, 169 mmol, 2.0 eq) at 0 °C. The reaction mixture was stirred at 100 °C for 10 h. After completion of the reaction by TLC, the reaction solution was poured into water (500 mL). The mixture was extracted with ethyl acetate (500 mL×2). The combined organic layer was washed with brine, dried over Na₂SO₄ and filtered. The filtration was concentrated under reduced pressure. The residue was purified by column chromatography (100-200 silica gel, 40% EA in PE as eluent) to afford compound 3 (13.0 g, 49%) as yellow solid.

TLC: PE/EA=3/1

R_{f} (Compound 1)=0.4

R_{f} (Compound 3)=0.3

LC-MS: 315.0 [M+1]⁺.

### Step 2: Synthesis of tert-butyl 3-(2-hydroxy-3,5-bis(trifluoromethyl)phenyl)-2-oxoimidazolidine-1-carboxylate

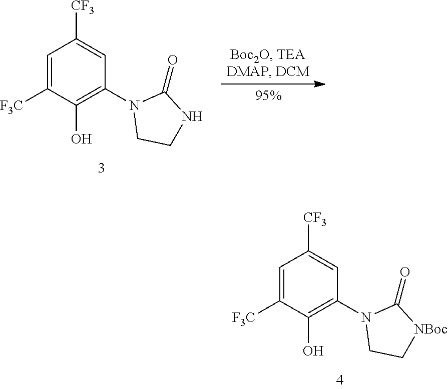 [SMILES: O=C1NCCN1c1cc(C(F)(F)F)cc(C(F)(F)F)c1O ; CC(C)(C)OC(=O)N1CCN(c2cc(C(F)(F)F)cc(C(F)(F)F)c2O)C1=O]

To a solution of compound 3 (8.0 g, 25.4 mmol, 1.0 eq) in DCM (100 mL) were added TEA (3.08 g, 30.5 mmol, 1.2 eq), Boc₂O (5.83 g, 26.7 mmol, 1.05 eq) and DMAP (310 mg, 2.54 mmol, 0.1 eq) at rt. The reaction mixture was stirred for 2 h at rt. After completion of the reaction by, the reaction solution was poured into water (300 mL). The solution was extracted with ethyl acetate (500 mL×2). The combined organic layer was washed with brine, dried over Na₂SO₄ and filtered. The filtrate was concentrated under reduced pressure to afford compound 4 (10 g, 95%) as brown solid.

TLC: DCM/MeOH=20/1

R_{f} (Compound 3)=0.6

R_{f} (Compound 4)=0.5

LC-MS: 412.9 [M−1]⁻.

### Step 3: Synthesis of tert-butyl 3-(2-(benzyloxy)-3,5-bis(trifluoromethyl)phenyl)-2-oxoimidazolidine-1-carboxylate

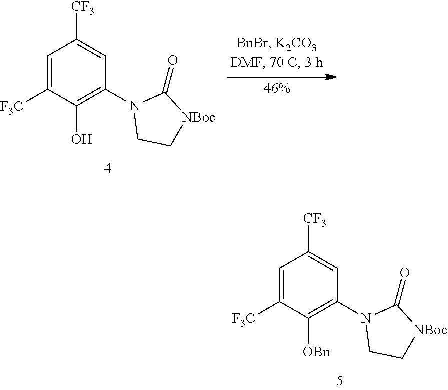 [SMILES: CC(C)(C)OC(=O)N1CCN(c2cc(C(F)(F)F)cc(C(F)(F)F)c2O)C1=O ; CC(C)(C)OC(=O)N1CCN(c2cc(C(F)(F)F)cc(C(F)(F)F)c2OCc2ccccc2)C1=O]

To a solution of compound 4 (10.0 g, 24.1 mmol, 1.0 eq) in DMF (100 mL) were added K₂CO₃ (5.03 g, 36.2 mmol, 1.5 eq) and BnBr (6.19 g, 36.2 mmol, 1.5 eq) at rt. The reaction mixture was stirred at 70 °C for 3 h. After completion of the reaction by TLC, the reaction solution was poured into water (300 mL). The solution was extracted with ethyl acetate (500 mL×2). The combined organic layer was washed with brine, dried over Na₂SO₄ and filtered. The filtrate was concentrated under reduced pressure. The residue was purified by column chromatography (100-200 silica gel, 5% EA in PE as eluent) to afford compound 5 (5.6 g, 46%) as yellow solid.

TLC: PE/EA=5/1

R_{f} (Compound 4)=0.1

R_{f} (Compound 5)=0.8

LC-MS: 449.0 [M+1-56]~; 405.0 [M+1-100]⁺.

### Step 4: Synthesis of 1-(2-(benzyloxy)-3,5-bis(trifluormethyl)phenyl)imidazolidine-2-one

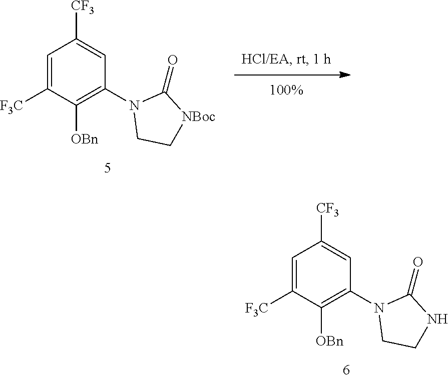 [SMILES: CC(C)(C)OC(=O)N1CCN(c2cc(C(F)(F)F)cc(C(F)(F)F)c2OCc2ccccc2)C1=O ; O=C1NCCN1c1cc(C(F)(F)F)cc(C(F)(F)F)c1OCc1ccccc1]

To a solution of compound 5 (8.2 g, 16.3 mmol, 1.0 eq) in ethyl acetate (10 mL) was added HCl/ethyl acetate (50 mL) at 0 °C. The reaction mixture was stirred at rt for 1 h. After completion of the reaction by, the reaction solution was concentrated under reduced pressure to afford compound 6 (6.7 g, 100%) as yellow solid.

TLC: PE/EA=5/1

R_{f} (Compound 5)=0.8

R_{f} (Compound 6)=0.2

LC-MS: 405.0 [M+1]+.

### Step 5: Synthesis of (S)-1-(2-benzyloxy)-3,5-bis(trifluoromethyl)phenyl-3-(oxiran-2-ylmethyl)imidazolidine-2-one

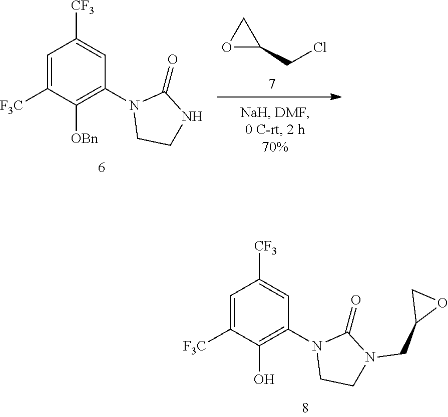 [SMILES: O=C1NCCN1c1cc(C(F)(F)F)cc(C(F)(F)F)c1OCc1ccccc1 ; ClC[C@@H]1CO1 ; O=C1N(C[C@H]2CO2)CCN1c1cc(C(F)(F)F)cc(C(F)(F)F)c1O]

To a solution of compound 6 (350 mg, 0.86 mmol, 1.0 eq) in DMF (5 mL) was added 60% NaH (86 mg, 2.16 mmol, 2.5 eq) at 0 °C. The reaction mixture was stirred at rt for 30 mins. The compound 7 (119 mg, 1.29 mmol, 1.5 eq) was added. The reaction mixture was stirred at rt for 1.5 h. After completion of the reaction by TLC, the reaction solution was poured into water (100 mL). The mixture was extracted with ethyl acetate (150 mL×2). The combined organic layer was washed with brine, dried over Na₂SO₄ and filtered. The filtrate was concentrated under reduced pressure. The residue was purified by column chromatography (100-200 silica gel, 45% EA in PE as eluent) to afford compound 8 (280 mg, 70%) as yellow oil.

TLC: PE/EA=3/1

R_{f} (Compound 6)=0.4

R_{f} (Compound 8)=0.5

LC-MS: 461.1 [M+1]⁺

### Step 6: Synthesis of (S)-1-(2-hydroxy-3,5-bis(trifluoromethyl)phenyl)-3-(oxiran-2-ylmethyl)imidazolidine-2-one

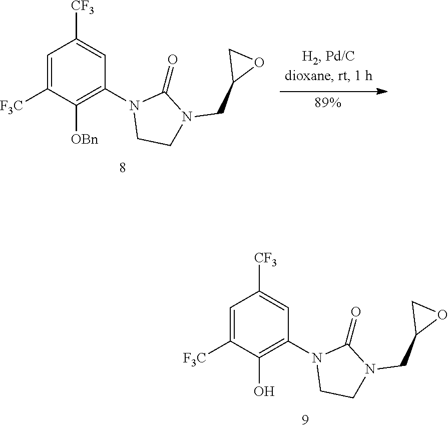 [SMILES: O=C1N(C[C@H]2CO2)CCN1c1cc(C(F)(F)F)cc(C(F)(F)F)c1OCc1ccccc1 ; O=C1N(C[C@H]2CO2)CCN1c1cc(C(F)(F)F)cc(C(F)(F)F)c1O]

To a solution of compound 8 (280 mg, 0.61 mmol, 1.0 eq) in dioxane (5 mL) was added Pd/C (56 mg, 20% wt) at rt. The reaction mixture was stirred for 1 h at rt. After completion of the reaction by TLC, the mixture was filtered with celite. The filtrate was concentrated under reduced pressure to afford compound 9 (200 mg, 89%) as colorless oil.

TLC: PE/EA=3/1

R_{f} (Compound 8)=0.5

R_{f} (Compound 9)=0.4

LC-MS: 371.2 [M+1]⁺.

### Step 7: Synthesis of (S)-2-(3-(oxiran-2-ylmethyl)-2-oxoimidazolidin-1-yl)-4,6-bis(trifluoromethyl)phenyl (4-fluorophenyl)(methyl-d₃)carbamate

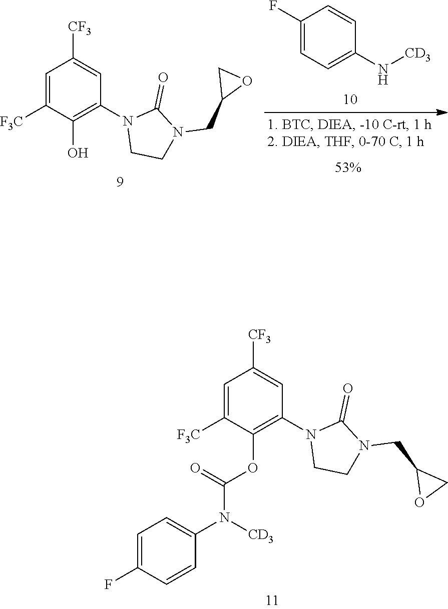 [SMILES: O=C1N(C[C@H]2CO2)CCN1c1cc(C(F)(F)F)cc(C(F)(F)F)c1O ; [2H]C([2H])([2H])Nc1ccc(F)cc1 ; [2H]C([2H])([2H])N(C(=O)Oc1c(N2CCN(C[C@H]3CO3)C2=O)cc(C(F)(F)F)cc1C(F)(F)F)c1ccc(F)cc1]

To a solution of compound 9 (200 mg, 0.54 mmol, 1.0 eq) in DCM (10 mL) were added DIEA (69 mg, 0.54 mmol, 1.0 eq) and triphosgene (160 mg, 0.54 mmol, 1.0 eq) at −10 °C. The reaction mixture was stirred at rt for 1 h. The reaction solution was concentrated under reduced pressure to remove the solvent. Then DCM (10 mL) was added to above residue. The solution was used for next step. To a solution of compound 10 (103 mg, 0.81 mmol, 1.5 eq) in DCM (10 mL) was added the above residue solution at 0 °C. The mixture was stirred at rt for 1 h. After completion of the reaction by TLC, the reaction solution was poured into water (100 mL). The mixture was extracted with DCM (150 mL×2). The combined organic layer was washed with brine, dried over Na₂SO₄ and filtered. The filtrate was concentrated under reduced pressure. The residue was purified by column chromatography (100-200 silica gel, 65% EA in PE as eluent) to afford compound 11 (150 mg, 53%) as yellow oil.

TLC: PE/EA=3/1

R_{f} (Compound 9)=0.4

R_{f} (Compound 11)=0.3

LC-MS: 525.3 [M+1]⁺.

### Step 8: Synthesis of (R)-2-(3-(3-(dimethylamino)-2-hydroxypropyl)-2-oxoimidazolidin-1-yl)-4,6-bis(trifluoromethyl)phenyl (4-fluoromethyl)(methyl-d₃)carbamate

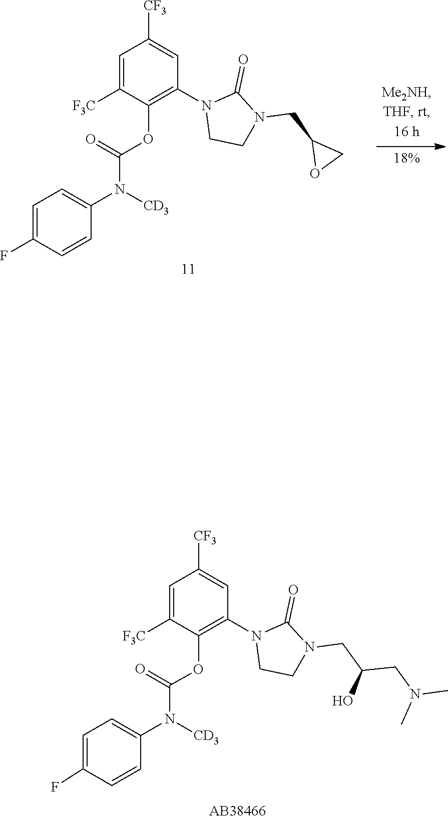 [SMILES: [2H]C([2H])([2H])N(C(=O)Oc1c(N2CCN(C[C@H]3CO3)C2=O)cc(C(F)(F)F)cc1C(F)(F)F)c1ccc(F)cc1 ; [2H]C([2H])([2H])N(C(=O)Oc1c(N2CCN(C[C@H](O)CN(C)C)C2=O)cc(C(F)(F)F)cc1C(F)(F)F)c1ccc(F)cc1]

To a solution of compound 11 (150 mg, 0.29 mmol, 1.0 eq) in THF (2 mL) at rt was added Me₂NH (0.85 mL 1.0 M/L in THF, 3.0 eq). The mixture was stirred for 16 h at rt. After completion of the reaction by TLC, the residue was purified by column chromatography (100-200 silica gel, 9.5% MeOH in DCM as eluent) to afford title compound (AB38466; 29.7 mg, 18%) as off-white solid.

TLC: DCM/MeOH=2/1

R_{f} (Compound 11)=0.5

R_{f} (AB38466)=0.3

¹H NMR (400 MHz, CD₃OD) δ 8.08 (br s, 1H), 7.83-7.90 (m, 1H), 7.40 (s, 2H), 7.15 (t, J=8.4 Hz, 2H), 3.95-3.70 (m, 5H), 3.38-3.33 (m, 1H), 3.25-3.19 (m, 1H), 2.39 (br s, 2H), 2.29-2.27 (m, 6H); LC-MS: 570.1 [M+Na]⁺.

### Example 51

### N-(4-fluorophenyl)-N-methyl-2-(2-(2-oxoimidazolidin-1-yl)-4,6-bis(trifluoromethyl)phenyl)acetamide

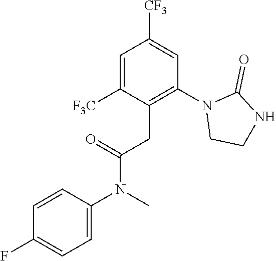 [SMILES: CN(C(=O)Cc1c(N2CCNC2=O)cc(C(F)(F)F)cc1C(F)(F)F)c1ccc(F)cc1]

### Step 1: Synthesis of diethyl 2-(2,4-bis(trifluoromethyl)phenyl)malonate

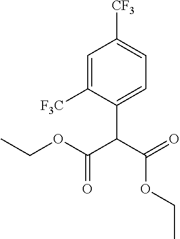 [SMILES: CCOC(=O)C(C(=O)OCC)c1ccc(C(F)(F)F)cc1C(F)(F)F]

A solution of 1-bromo-2,4-bis(trifluoromethyl)benzene (2 g, 0.0068 mol), Cs₂CO₃ (8.89 g, 0.027 mol), CuI (0.64 g, 0.0034 mol) and 1,10-phenanthroline (1.2 g, 0.0068 mol) in 1,4 dioxane (20 mL) was purged with N2 in a sealed tube for 10 min and then added diethylmalonate (2.18 g, 0.0136 mol), purged with N2 for an another 10 min. The tightly closed sealed tube was heated to 90 °C in an oil bath for 16 h with stirring. Progress of the reaction was monitored by TLC (Rf=0.5, 10% EtOAc in Hexane). After completion of the reaction, the reaction mixture quenched with ice cold water (50 mL) followed by extraction with EtOAc (2×50 mL). The organic layer collected was dried over anhydrous Na₂SO₄, concentrated under reduced pressure to afford the crude. The crude was purified by column chromatography (100-200 silica gel, 5-7% EtOAc in Hexane as eluent) to afford the title compound as colour less liquid (0.85 g, 34% yield).

### Step 2: Synthesis of ethyl 2-(2,4-bis(trifluoromethyl)phenyl)acetate

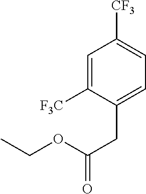 [SMILES: CCOC(=O)Cc1ccc(C(F)(F)F)cc1C(F)(F)F]

A solution of diethyl 2-(2,4-bis(trifluoromethyl)phenyl)malonate (0.3 g, 0.0008 mol), NaCl (0.46 g, 0.008 mol, water (2 mL) and DMSO (9 mL) in microwave vial (30 mL, CEM) was heated at 190 °C under microwave condition for 20 min (CEM, Power: 150-200, Pressure: 15-17 psi internal). After completion of the reaction confirmed by TLC (R_{f} 0.4, 10% EtOAc in Hexane), the reaction mixture quenched with ice cold water (50 mL), followed by extraction with EtOAc (2×50 mL). The organic layer collected was dried over anhydrous Na₂SO₄ and concentrated under reduced pressure to afford the crude. The crude obtained was purified by column chromatography (100-200 silica gel, 2-4% EtOAc in Hexane as eluent) to afford the title compound as colourless liquid (0.02 g, 8.3% yield).

### Step 3: Synthesis of ethyl 2-(2-bromo-4,6-bis(trifluoromethyl)phenyl)acetate

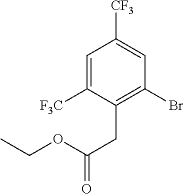 [SMILES: CCOC(=O)Cc1c(Br)cc(C(F)(F)F)cc1C(F)(F)F]

A solution of ethyl 2-(2,4-bis(trifluoromethyl)phenyl)acetate (2.0 g, 0.0066 mol), Na₂S₂O₈ (4.76 g, 0.0199 mol), trifluoromethanesulfonic acid (8.32 g, 5.17 mL, 0.033 mol), 1,2-dichlorethane (30 mL) in seal tube was purged with N2 for 15 min. Added Pd(OAc)₂ (0.149 g, 0.00066 mol) followed by NBS (2.37 g, 0.0133 mol) at once and then heated the tightly sealed tube at 80 °C in an oil bath for 2 h. After completion of the reaction (TLC: R_{f} −0.6, 10% EtOAc in Hexane), the reaction mixture was concentrated under reduced pressure to afford the crude. The crude obtained was purified by column chromatography (100-200 silica gel, 3-6% EtOAc in Hexane as eluent) to afford the title compound as colourless liquid (0.35 g, 14% yield).

### Step 4: Synthesis of ethyl 2-(2-(2-oxoimidazolidin-1-yl)-4,6-bis(trifluoromethyl)phenyl)acetate

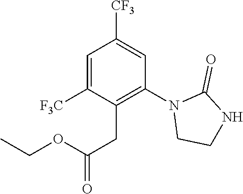 [SMILES: CCOC(=O)Cc1c(N2CCNC2=O)cc(C(F)(F)F)cc1C(F)(F)F]

A solution of ethyl 2-(2-bromo-4,6-bis(trifluoromethyl)phenyl)acetate (0.03 g, 0.000079 mol), 2-imidazoilidinoe (0.013 g, 0.00016 mol), K₂CO₃ (0.022 g, 0.00015 mol) and CsF (0.024 g, 0.00015 mol) in 1,4 dioxane (3 mL) was purged with N2 in a sealed tube for 10 min and then added CuI (0.0045 g, 0.000023 mol) followed by DMEDA (0.0034 g, 0.004 mL, 0.000039 mol); again purged with N2 for 10 min. The tightly closed sealed tube was heated to 90 °C in an oil bath for 16 h with stirring. Progress of the reaction was monitored by TLC (Rf=0.3, 60% EtOAc in Hexane). After completion of the reaction, the reaction mixture was concentrated under reduced pressure to afford the crude. The crude was purified by preparative TLC (solid phase: merck, 20×20 cm, silicagel 60 GF₂₅₄, 1 mm, PLC glass plate, 60% EtOAc in Hexane as eluent) to afford the title compound as white solid (4 mg, 13%)

### Step 5: Synthesis of 2-(2-(2-oxoimidazolidin-1-yl)-4,6-bis(trifluoromethyl)phenyl)acetic acid

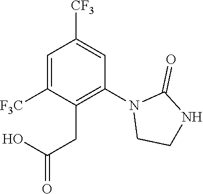 [SMILES: O=C(O)Cc1c(N2CCNC2=O)cc(C(F)(F)F)cc1C(F)(F)F]

A solution of ethyl 2-(2-(2-oxoimidazolidin-1-yl)-4,6-bis(trifluoromethyl)phenyl)acetate, (0.025 g, 0.00006 mol) in THF:MeOH:H₂O (2:2:1, 5 mL) was cooled to 0 °C in an ice bath for 10 min. To the above solution added LiOH·H₂O (0.001 g, 0.00018 mol) and continued stirring at ambient temperature for 18 hours. After completion of the reaction was confirmed by TLC (R_{f} −0.2, 10% MeOH in DCM), concentrated the reaction mixture under reduced pressure to afford the title compound as white solid (0.01 g, 40%).

### Step 6: Synthesis of N-(4-fluorophenyl)-N-methyl-2-(2-(2-oxoimidazolidin-1-yl)-4,6-bis(trifluoromethyl)phenyl)acetamide

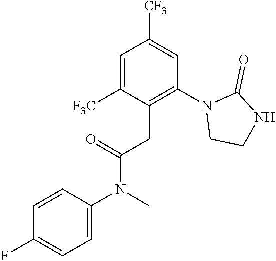 [SMILES: CN(C(=O)Cc1c(N2CCNC2=O)cc(C(F)(F)F)cc1C(F)(F)F)c1ccc(F)cc1]

To solution of 2-(2-(2-oxoimidazolidin-1-yl)-4,6-bis(trifluoromethyl)phenyl)acetic acid (0.05 g, 0.00014 mol), in DCM (5 mL), added 4-fluoro-N-methyl aniline (0.017 g, 0.017 mL, 0.00014 mol), followed by DMTMM (0.039 g, 0.00014 mol) and stirring was continued at ambient temperature for 4 h. After completion of the reaction was confirmed by TLC (R_{f} 0.4, 5% MeOH in DCM), quenched the reaction mixture with cold water (15 mL) and extracted with DCM (2×15 mL). The organic layer separated was combined, dried over anhydrous Na₂SO₄ and concentrated under reduced pressure to afford the crude. The crude obtained was purified by preparative TLC (solid phase: merck, 20×20 cm, silicagel 60 GF₂₅₄, 1 mm, PLC glass plate, 60% EtOAc in Hexane as eluent) to afford the title compound as white solid (18 mg, 27%).

¹H NMR (500 MHz, DMSO-d₆) δ8.10 (s, 1H), 7.90 (s, 1H), 7.45 (m, 2H), 7.33 (t, J=8.6 Hz, 2H), 7.07 (s, 1H), 3.79 (t, J=6.5 Hz, m, 2H), 3.61 (s, 2H), 3.49 (t, J=7.6 Hz, 2H), 3.17 (s, 3H); MS(ESI): m/z 464.40 (M+H)⁺.

### Example 52

### 2-(2-oxoimidazolidin-1-yl)-4,6-bis(trifluoromethyl)phenyl (5-fluoropyridin-2-yl)(methyl)carbamate

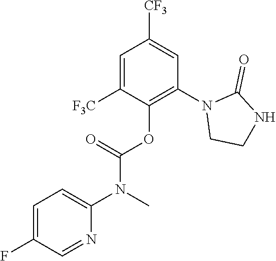 [SMILES: CN(C(=O)Oc1c(N2CCNC2=O)cc(C(F)(F)F)cc1C(F)(F)F)c1ccc(F)cn1]

### Step 1: Synthesis of (5-fluoropyridin-2-yl)(methyl)carbamic chloride

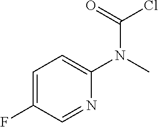

To a solution of 5-fluoro-N-methylpyridin-2-amine (0.096 g, 0.76 mmol) and pyridine (0.180 g, 2.28 mmol) in 5 mL of dichloromethane at 0 °C, triphosgene (0.113 g, 0.381 mmol) dissolved in 3 mL dichloromethane was added dropwise under inert atmosphere. The reaction was stirred at room temperature for 2 h. The reaction mixture was concentrated to a solid under reduced pressure.

### Step 2: Synthesis of 2-(2-oxoimidazolidin-1-yl)-4,6-bis(trifluoromethyl)phenyl (5-fluoropyridin-2-yl)(methyl)carbamate

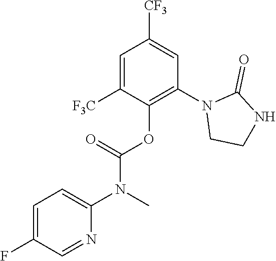 [SMILES: CN(C(=O)Oc1c(N2CCNC2=O)cc(C(F)(F)F)cc1C(F)(F)F)c1ccc(F)cn1]

To a solution of 1-(3,5-bis(trifluoromethyl)-2-hydroxyphenyl)imidazolidin-2-one (0.20 g, 0.63 mmol) in 10 mL pyridine the crude oil obtained from step 1 was added and stirred under inert atmosphere at 90 °C for 18 h. The reaction was cooled to room temperature and concentrated to an oil under reduced pressure. The oil obtained was purified on 24 g silica gel cartridge using 0-100% ethyl acetate as eluent to afford 0.186 g of the title compound as a white solid.

¹H NMR (400 MHz, CDCl₃) δ 8.29, (d, J=4.0 Hz, 1H)), 7.87 (d, J=4.0 Hz, 1H)), 7.82 (s, 1H), 7.73 (d, J=4.0 Hz 1H), 7.43 (m, 1H), 3.91 (bs, 2H), 3.47 (m, 5H); MS(ESI): m/z 467.22 (M+H)⁺.

### Example 53

### 2-(2-oxoimidazolidin-1-yl)-4,6-bis(trifluoromethyl)phenyl (5-fluoro-6-methylpyridin-2-yl)(methyl)carbamate

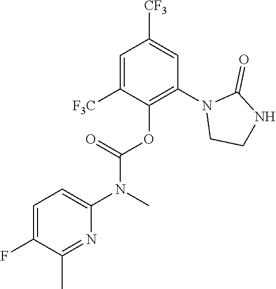 [SMILES: Cc1nc(N(C)C(=O)Oc2c(N3CCNC3=O)cc(C(F)(F)F)cc2C(F)(F)F)ccc1F]

### Step 1: Synthesis of (5-fluoro-6-methylpyridin-2-yl)carbamic chloride

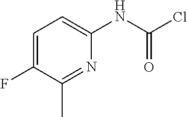

To a solution of 5-fluoro-N-methylpyridin-2-amine (0.23 g, 0.159 mmol) and pyridine (0.37 g, 0.477 mmol) in 5 mL of dichloromethane at 0 °C, triphosgene (0.024 g, 0.0795 mmol) dissolved in 3 mL dichloromethane was added dropwise under inert atmosphere. The reaction was stirred at room temperature for 2 h. The reaction mixture was concentrated to a solid under reduced pressure.

### Step 2: Synthesis of 2-(2-oxoimidazolidin-1-yl)-4,6-bis(trifluoromethyl)phenyl (5-fluoro-6-methylpyridin-2-yl)(methyl)carbamate

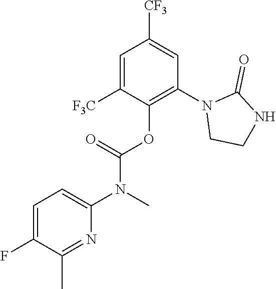 [SMILES: Cc1nc(N(C)C(=O)Oc2c(N3CCNC3=O)cc(C(F)(F)F)cc2C(F)(F)F)ccc1F]

To a solution of 1-(3,5-bis(trifluoromethyl)-2-hydroxyphenyl)imidazolidin-2-one (0.050 g, 0.159 mmol) in 10 mL pyridine the crude oil obtained from step 1 was added and stirred under inert atmosphere at 90 °C for 18 h. The reaction was cooled to room temperature and concentrated to an oil under reduced pressure. The oil obtained was purified on 24 g silica gel cartridge using 0-100% ethyl acetate as eluent to afford 0.016 g of the title compound as an oil.

¹H NMR (400 MHz, CDCl₃) δ 7.88 (d, J=4.0 Hz, 1H)), 7.81 (s, 1H), 7.36 (s, 1H), 7.34 (t, J=8.0 Hz 1H), 3.91 (bs, 2H), 3.57 (m, 5H); MS(ESI): m/z 481.25 (M+H)⁺.

### Example 54

### 4-chloro-2-(3-(2-hydroxyethyl)-2-oxoimidazolidin-1-yl)-6-(trifluoromethyl)phenyl (4-fluorophenyl)(methyl)carbamate

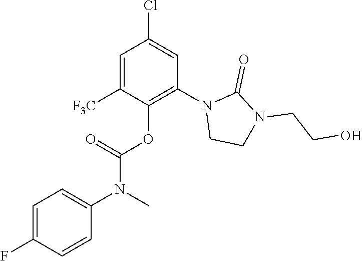 [SMILES: CN(C(=O)Oc1c(N2CCN(CCO)C2=O)cc(Cl)cc1C(F)(F)F)c1ccc(F)cc1]

### Step 1: Synthesis of (4-Fluorophenyl)(methyl)carbamic chloride

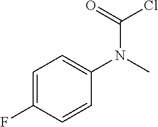

To a solution of 4-fluoro-N-methylaniline (0.58 g, 4.66 mmol) and pyridine (0.73 g, 9.34 mmol) in 12 mL of dichloromethane at 0 °C, triphosgene (0.69 g, 2.33 mmol) dissolved in 6 mL dichloromethane was added dropwise under inert atmosphere. The reaction was stirred at room temperature for 2 h. The reaction mixture was diluted with 20 mL dichloromethane and extracted with 20 mL 1N HCl. The organic layer was separated, dried over with anhydrous sodium sulfate, filtered and concentrated to a solid under reduced pressure. ¹H NMR (400 MHz, CDCl₃) δ 7.15 (m, 2H), 7.04 (m, 2H), 3.3 (s, 3H); MS(ESI): m/z 188.0 (M+H)⁺.

### Step 2: Synthesis of 4-chloro-2-iodo-6-(trifluoromethyl)phenyl (4-fluorophenyl)(methyl)carbamate

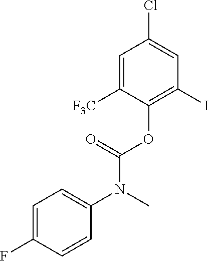 [SMILES: CN(C(=O)Oc1c(I)cc(Cl)cc1C(F)(F)F)c1ccc(F)cc1]

4-chloro-2-iodo-6-(trifluoromethyl)phenol (200 mg, 0.62 mmol) and (4-fluorophenyl) (methyl)carbamic chloride (179 mg, 0.745 mmol) were dissolved into anhydrous pyridine (5 mL). This solution was stirred at 90 °C for 4 hours. The reaction was cooled to room temperature and concentrated down. The residual solid was partitioned between EtOAc and 1N aqueous HCL. The aqueous phase was separated and extracted with EtOAc twice. The combined organic extracts were washed with brine, dried over anhydrous sodium sulfate and concentrated. The crude product was purified by column chromatography on silica gel using a gradient solvent system of 0 to 20% of ethyl acetate in hexanes to afford the titled compound as a white solid (225 mg 76%). ESIMS: m/z 473.98 [(M+H)⁺]

### Step 3: Synthesis of 4-chloro-2-(3-(2-hydroxyethyl)-2-oxoimidazolidin-1-yl)-6-(trifluoromethyl)phenyl (4-fluorophenyl)(methyl)carbamate

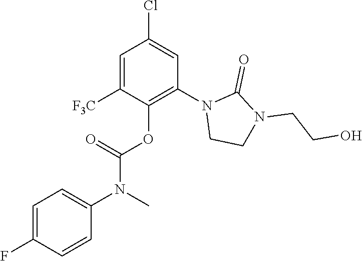 [SMILES: CN(C(=O)Oc1c(N2CCN(CCO)C2=O)cc(Cl)cc1C(F)(F)F)c1ccc(F)cc1]

4-chloro-2-iodo-6-(trifluoromethyl)phenyl (4-fluorophenyl)(methyl)carbamate (215 mg, 0.454 mmol), 1-(2-hydroxyethyl) 2-imidazolidone (118 mg, 0.909 mmol), copper (I) iodide (43 mg, 0.22 mmol), cesium fluoride (138 mg, 0.909 mmol), N,N′-dimethyl ethylenediamine (0.50 mmol, 50 μL, 0.454 mmol) and anhydrous powered potassium carbonate (0.126 gmg, 0.909 mmol) were added to degassed anhydrous 1,4-dioxane (10.0 mL) under nitrogen. The resulting suspension was stirred at 90 °C overnight. The reaction was cooled to room temperature and concentrated down to yield a semi-solid. The crude product was purified by column chromatography on silica gel using a gradient solvent system of 0 to 3% of methanol in dichloromethane to afford the titled compound as a white solid (54 mg, 25%).

¹H NMR (400 MHz, CDCl₃) δ 7.35 (m, 2H), 7.28 (m, 2H), 7.08 (t, J=8.0 Hz, 2H), 3.84 (s, 3H), 3.72 (s, 1H), 3.58 (t, J=4.0 Hz, 2H), 3.44 (bs, 4H), 3.34 (s, 2H); ESIMS: m/z 476.21 [(M+H)⁺].

### Example 55

### 2,4-dichloro-6-(3-(2-hydroxyethyl)-2-oxoimidazolidin-1-yl)phenyl (4-fluorophenyl)(methyl)carbamate

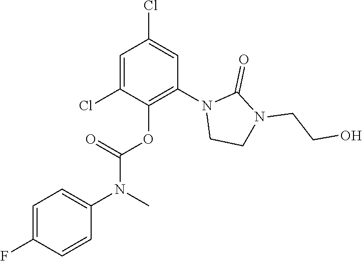 [SMILES: CN(C(=O)Oc1c(Cl)cc(Cl)cc1N1CCN(CCO)C1=O)c1ccc(F)cc1]

### Step 1: 2,4-dichloro-6-iodophenyl (4-fluorophenyl)(methyl)carbamate

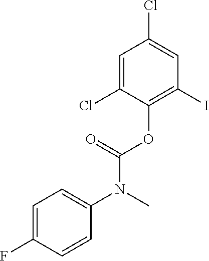 [SMILES: CN(C(=O)Oc1c(Cl)cc(Cl)cc1I)c1ccc(F)cc1]

2,4-dichloro-6-iodophenol (500 mg, 1.73 mmol) and (4-fluorophenyl) (methyl)carbamic chloride (498 mg, 2.00 mmol) were dissolved into anhydrous pyridine (8 mL). This solution was stirred at 90 °C for 4 hours. The reaction was cooled to room temperature and concentrated down. The residual solid was partitioned between EtOAc and 1N aqueous HCl. The aqueous phase was separated and extracted with EtOAc twice. The combined organic extracts were washed with brine, dried over anhydrous sodium sulfate and concentrated. The crude product was purified by column chromatography on silica gel using a gradient solvent system of 0 to 20% of ethyl acetate in hexanes to afford the titled compound as a white solid (540 mg, 84%). ESIMS: m/z 439.99 [(M+H)⁺].

### Step 2: Synthesis of 2,4-dichloro-6-(3-(2-hydroxyethyl)-2-oxoimidazolidin-1-yl)phenyl (4-fluorophenyl)(methyl)carbamate

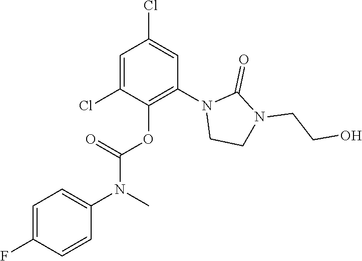 [SMILES: CN(C(=O)Oc1c(Cl)cc(Cl)cc1N1CCN(CCO)C1=O)c1ccc(F)cc1]

2,4-dichloro-6-iodophenyl (4-fluorophenyl)methyl)carbamate (223 mg, 0.50 mol), 1-(2Hydroxyethyl) 2-imidazolidone (0.101 mmol, 132 mg, 0.101 mmol), copper (I) iodide (48 mg, 0.25 mmol), cesium fluoride (154 mg, 0.101 mmol), N,N′-dimethyl ethylenediamine (56 μL, 0.50 mmol) and anhydrous powered potassium carbonate (48 mg, 0.101 mmol) were added to degassed anhydrous 1,4-dioxane (12.0 mL) under nitrogen. The resulting suspension was stirred at 90 °C overnight. The reaction was cooled to room temperature and concentrated down to yield a semi-solid. The crude product was purified by column chromatography on silica gel using a gradient solvent system of 0 to 3% of methanol in dichloromethane to afford the titled compound as a white solid (60 mg, 28%).

¹H NMR (400 MHz, CDCl₃) δ 7.35 (m, 4H), 7.08 (t, J=12.0 Hz, 2H), 3.84 (m, 2H), 3.62 (bs, 2H), 3.54 (bs, 3H), 3.42 (t, J=4.0 Hz, 2H), 3.38 (bs, 2H); ESIMS: m/z 442.15

### Example 56

### Biological Data

The following Cy5 fluorescence assay was used to measure the ability of compounds to inhibit Polq polymerase domain in vitro. The fluorescent based assay was performed as follows: 60 nM of the pre-annealed primer-template containing a 5′ Cy5 fluorophore conjugated template strand (SEQ ID NO:3: 5′-/5Cy5/CACTGTGAGCTTAGTCACATTTCATCATGCAGGACAG-3′), a downstream complementary oligo conjugated with a 3′ Blackhole quencher (SEQ ID NO:4: 5′-CTAAGCTCACAGTG/3IAbRQSp/-3′) and a primer strand (SEQ ID NO:5: 5′-CTGTCCTGCATGATG-3′) was mixed with 50 uM 2′-deoxyribonucleoside triphosphates (dNTPs), 0.1 mg/mL Bovine serum albumin (BSA), 0.01% NP-40, 10% glycerol, 1 mM dithiothreitol (DTT), 10 mM MgCl₂, 25 mM TrisHCl pH 7.8 in the presence of 2.5% DMSO with or without various concentrations (7-point dilution series) of Polq small molecule inhibitors represented by Formula I at 37 °C in a volume of 40 μL. The reactions were initiated by the addition of 5 nM of purified recombinant human Polq polymerase domain (comprising amino acid residues 1792-2590 (SEQ ID NO: 1). The reactions were terminated by the addition of 20 mM EDTA after 18 min, and the Cy5 fluorescence intensity was measured using a Clariostar (BMG Labtech) plate reader. Reactions were performed in triplicate and the % inhibition at each concentration of the respective compound of Formula I was based on the mean. The compounds of Examples 1 to 10 were tested in the above Polq polymerase enzymatic activity assay. The IC50 of each compound represents the average concentration of compound that resulted in 50% inhibition of Polq polymerase enzymatic activity which was determined from a scatter plot (% inhibition versus compound concentration) curve generated by PRISM software for each compound inhibition data set.

| Example number | IC₅₀ (nM) Polq |
|---|---|
| 1 | 12 |
| 2 | 4.5 |
| 3 | 6 |
| 4 | 3.4 |
| 5 | 6 |
| 6 | 3.2 |
| 7 | 4 |
| 8 | 3.4 |
| 9 | 21 |
| 10 | 21 |
| 11 | 15 |
| 12 | 4.4 |
| 13 | 29 |
| 14 | 45 |
| 15 | 80 |
| 16 | 5.4 |
| 17 | 1000 |
| 18 | 22 |
| 19 | 16 |
| 20 | 2.4 |
| 21 | 1.9 |
| 22 | 5.6 |
| 23 | 1.8 |
| 24 | 6.7 |
| 25 | 5.8 |
| 26 | 48 |
| 27 | 24 |
| 28 | 1200 |
| 29 | 18000 |
| 30 | 2.2 |
| 31 | 31 |
| 32 | 2.3 |
| 33 | 590 |
| 34 | 2 |
| 35 | 2.5 |
| 36 | 6 |
| 37 | 2.7 |
| 38 | 7.8 |
| 39 | 3.8 |
| 40 | 6.3 |
| 41 | 5.5 |
| 42 | 11.7 |
| 43 | 5.4 |
| 44 | 7 |
| 45 | 6.1 |
| 46 | 5.5 |
| 47 | 4.8 |
| 48 | 5.1 |
| 49 | 7.5 |
| 50 | 5.1 |
| 51 | 117 |
| 52 | 11.4 |
| 53 | 2.7 |
| 54 | 68 |
| 55 | 133 |

Synergistic Co-Administration with PARP Inhibitors

To investigate the suitability of Polq inhibitors for combinatorial therapy, various cell lines were treated with various concentrations of a PARP inhibitor with or without the compound of Example 6 at various concentrations. As a control, cell survival was normalized to DMSO-treated cells. Cells harboring BRCA2 mutations (PE01, VC8, and CAPAN-1) as well as cells that were BRCA2-null (HCT116 and DLD1) were examined, with olaparib as the PARP inhibitor. MDA-MB-231 triple-negative breast cancer cells were also examined with talazoparib as the PARP inhibitor. Synergy between the compound of Example 6 and PARP inhibitors was observed for cell types harboring BRCA2 mutations as determined by Combenefit software analysis of clonogenic survival data.

Polθ₁₇₉₂₋₂₅₉₀
Amino acid sequence:
(SEQ ID NO: 1)
GFKDNSPISDTSFSLQLSQDGLQLTPASSSSESLSIIDVASDQNL
FQTFIKEWRCKKRFSISLACEKIRSLTSSKTATIGSRFKQASSPQ
EIPIRDDGFPIKGCDDTLVVGLAVCWGGRDAYYFSLQKEQKHSEI
SASLVPPSLDPSLTLKDRMWYLQSCLRKESDKECSVVIYDFIQSY
KILLLSCGISLEQSYEDPKVACWLLDPDSQEPTLHSIVTSFLPHE
LPLLEGMETSQGIQSLGLNAGSEHSGRYRASVESILIFNSMNQLN
SLLQKENLQDVFRKVEMPSQYCLALLELNGIGFSTAECESQKHIM
QAKLDAIETQAYQLAGHSFSFTSSDDIAEVLFLELKLPPNREMKN
QGSKKTLGSTRRGIDNGRKLRLGRQFSTSKDVLNKLKALHPLPGL
ILEWRRITNAITKVVFPLQREKCLNPFLGMERIYPVSQSHTATGR
ITFTEPNIQNVPRDFEIKMPTLVGESPPSQAVGKGLLPMGRGKYK
KGFSVNPRCQAQMEERAADRGMPFSISMRHAFVPFPGGSILAADY
SQLELRILAHLSHDRRLIQVLNTGADVFRSIAAEWKMIEPESVGD
DLRQQAKQICYGIIYGMGAKSLGEQMGIKENDAACYIDSFKSRYT
GINQFMTETVKNCKRDGFVQTILGRRRYLPGIKDNNPYRKAHAER
QAINTIVQGSAADIVKIATVNIQKQLETFHSTFKSHGHREGMLQS
DQTGLSRKRKLQGMFCPIRGGFFILQLHDELLYEVAEEDVVQVAQ
IVKNEMESAVKLSVKLKVKVKIGASWGELKDFDV.

1. A compound having the structure of Formula (I), or a tautomeric or a stereochemically isomeric form, a pharmaceutically acceptable salt or a solvate thereof: 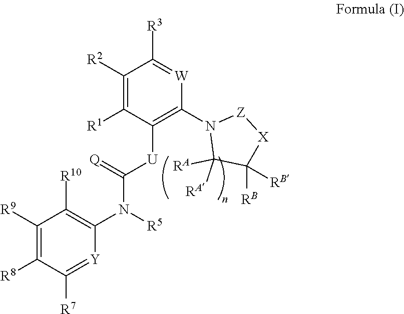
wherein:
U represents CH₂, O, S, or NR^{U};
W represents C(R⁴) or N;
Y represents C(R⁶) or N;
Q represents O or S;
R¹, R², R³, R⁴, R⁶, R⁷, R⁸, R⁹, and R¹⁰ independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, NR^{X}R^{Y}, and combinations thereof, wherein two adjacent groups R¹ to R⁴ or R⁶ to R¹⁰ optionally join to form a 5- to 7-membered saturated or unsaturated ring optionally containing one or more heteroatoms selected from O, N or S;
R⁵ and R^{U} independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof,
Z represents CR^{Z}R^{Z′}, C═S, or C═O;
X represents C(R¹⁵)(R¹⁶), N(R¹⁷) or O;
R¹⁵, R¹⁶, and R¹⁷ independently represent hydrogen, deuterium, C₁₋₆ alkyl, haloC₁₋₆ alkyl, —OR^{15a}, —SR^{15a}, nitrile, —COC₁₋₆ alkyl, —COOC₁₋₆ alkyl, hydroxy, C₁₋₆ alkoxy, C₁₋₆ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof; wherein R¹⁵, R¹⁶, and R¹⁷ may further comprise one or more divalent linkers L selected from the group consisting alkylene, cycloalkylene, heteroalkylene, heterocycloalkylene, alkenylene, alkynylene, arylene, heteroarylene, silyl, amine, amide, ester, ether, carbonyl, carbamate, sulfamate, sulfonic ester, sulfoximine, sulfonamide, thioether, thioester, disulfide, hydrazine, urea, thiourea, phosphate, phosphonate ester, poly(alkyl ether), heteroatom, and combinations thereof, wherein R¹⁵ and R¹⁶ may be taken together to form a ring;
n is 0, 1, or 2; wherein when n is 0, N is directly bonded to the carbon having R^{B} and R^{B′} bound thereto;
each R^{A}, R^{A′}, R^{Z}, and R^{Z′} independently represents hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof,
R^{B} and R^{B′} independently represents hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof, or R^{B} and the carbon to which it is bound together form a carbonyl group and R^{B′} is not present;
R^{15a} and R^{15b} independently represent hydrogen, deuterium, or C₁₋₆ alkyl; wherein two groups R^{15a} and R^{15b}, or two groups R^{15b}, may join together to form a 5 to 7 membered saturated ring system which may be optionally substituted by one or more C₁₋₆ alkyl groups;
R^{V}, R^{W}, R^{X} and R^{Y} independently represent hydrogen, deuterium, C₁₋₆ alkyl, haloC₁₋₆ alkyl, C₃₋₈ cycloalkyl, —COC₁₋₆ alkyl or heterocyclyl; wherein said alkyl groups may be optionally substituted with or more deuterium, hydroxy, amino or sulfone groups; and said heterocyclyl ring may be optionally substituted by one or more deuterium, oxo, hydroxy, C₁₋₆ alkanol or —COC₁₋₆ alkyl groups.

2. The compound of claim 1, wherein U is O H or

3. (canceled)

4. (canceled)

5. The compound of claim 1, wherein Q is O.

6. The compound of claim 1, wherein Q is S.

7. The compound of claim 1, wherein at least one of conditions (i) to (iii) is satisfied:
(i) at least one of R¹ and R³ represents halogen or haloC₁₋₆ alkyl;
(ii) R² is selected from the group consisting of hydrogen, deuterium, halogen, C₁₋₆ alkyl, and haloC₁₋₆ alkyl; and
(iii) R⁴ is selected from the group consisting of hydrogen, deuterium, halogen, nitrile, methyl, and ethynyl.

8. The compound of claim 1, wherein R¹ and R³ each independently represents halogen or haloC₁₋₆ alkyl.

9. The compound of claim 1, wherein R¹ and R³ each represent CF₃.

10. (canceled)

11. (canceled)

12. The compound of claim 1, wherein R⁵ represents CH₃ or CD₃; or
R⁵ represents: 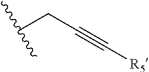
wherein R^{5′} is selected from the group consisting of hydrogen, deuterium, C₁₋₆ alkyl, C₂₋₆ alkenyl, alkynl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y} aryl, heteroaryl, heterocyclyl, amide, and combinations thereof.

13. (canceled)

14. The compound of claim 1, wherein R⁵ represents one of the following substituents:

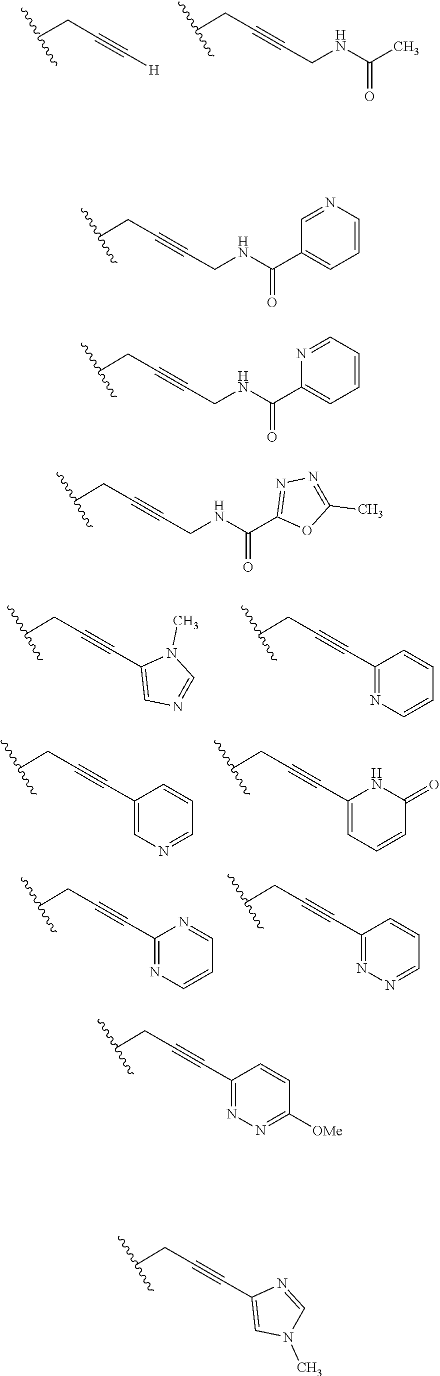

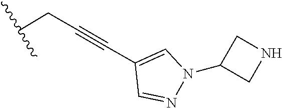 [SMILES: *CC#Cc1cnn(C2CNC2)c1]

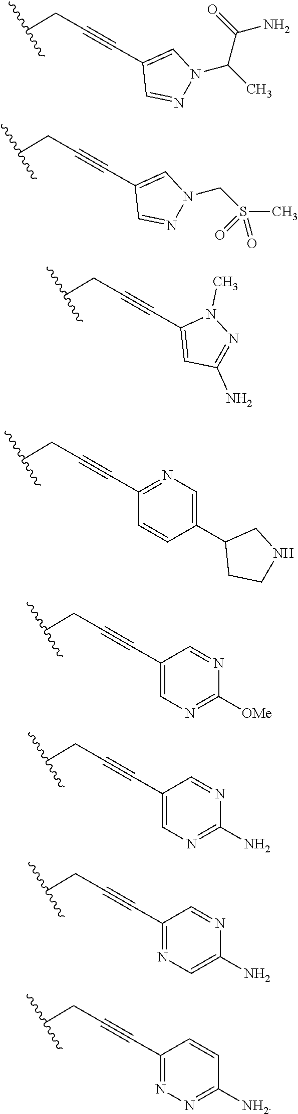

15. The compound of claim 1, wherein R⁸, R⁹, and R¹⁰ each represent halogen.

16. The compound of claim 1, wherein at least one of the following conditions (i) to (iv) is satisfied:
(i) R⁷ represents hydrogen, fluorine, chlorine, CH₃, or CD₃;
(ii) R⁸ represents fluorine;
(iii) R⁹ represents chlorine;
(iv) R¹⁰ represents fluorine.

17. (canceled)

18. The compound of claim 16, wherein Y is N.

19. (canceled)

20. (canceled)

21. The compound of claim 1, wherein R⁷ and R⁸ or R⁸ and R⁹ join to form a pyrrolyl ring which is optionally substituted.

22. (canceled)

23. The compound of claim 1, wherein at least one of conditions (i) to (iii) is satisfied:
(i) R^{A} and R^{A′} each represent hydrogen or deuterium;
(ii) Z represents C═O or C═S
(iii) Z represents CR^{Z}R^{Z′}.

24.-26. (canceled)

27. The compound of claim 1, wherein X represents NR¹⁷ and R¹⁷ represents a substituent selected from the group consisting of:

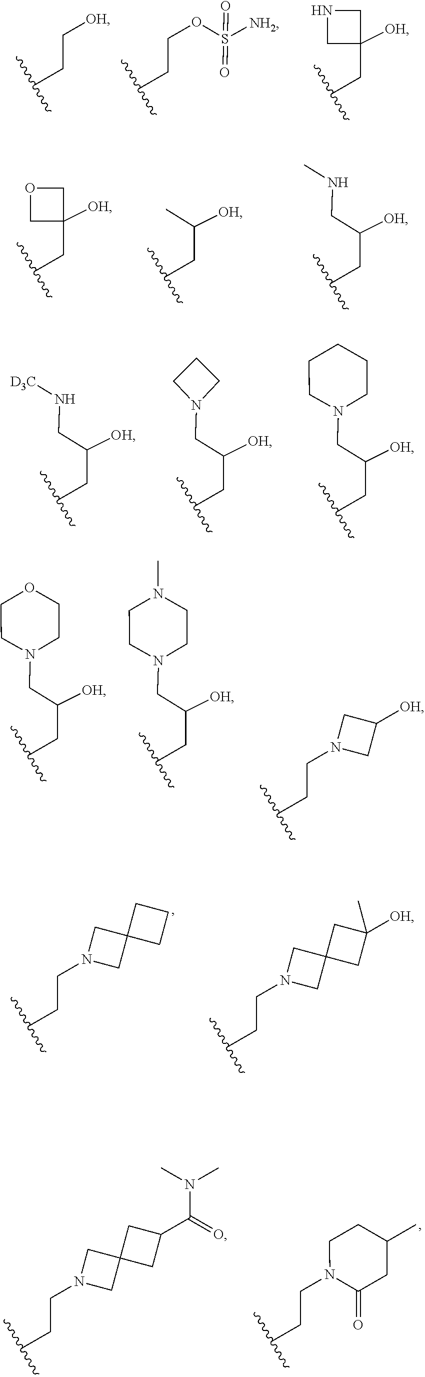

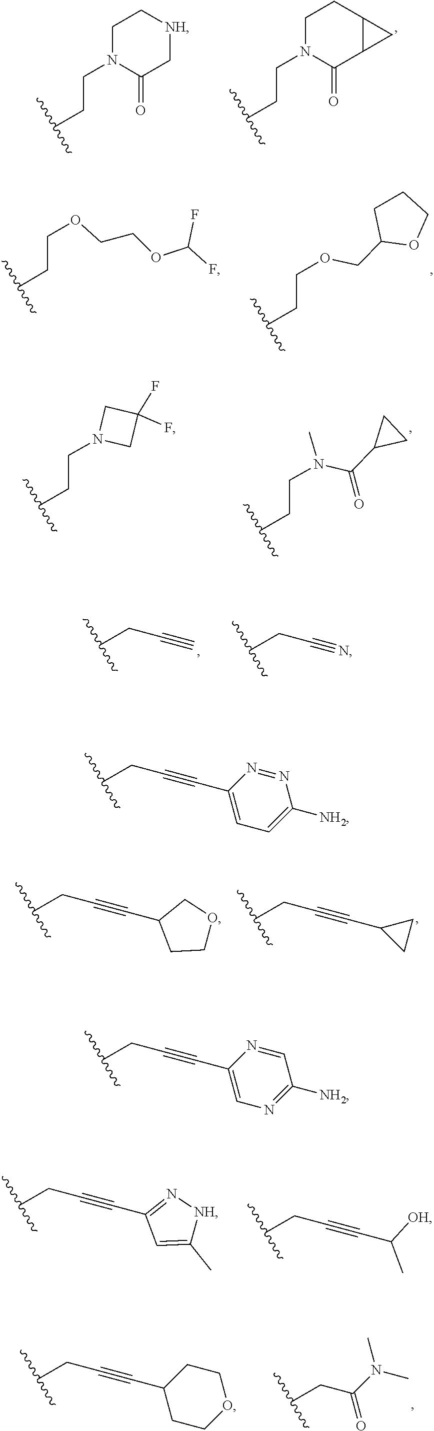

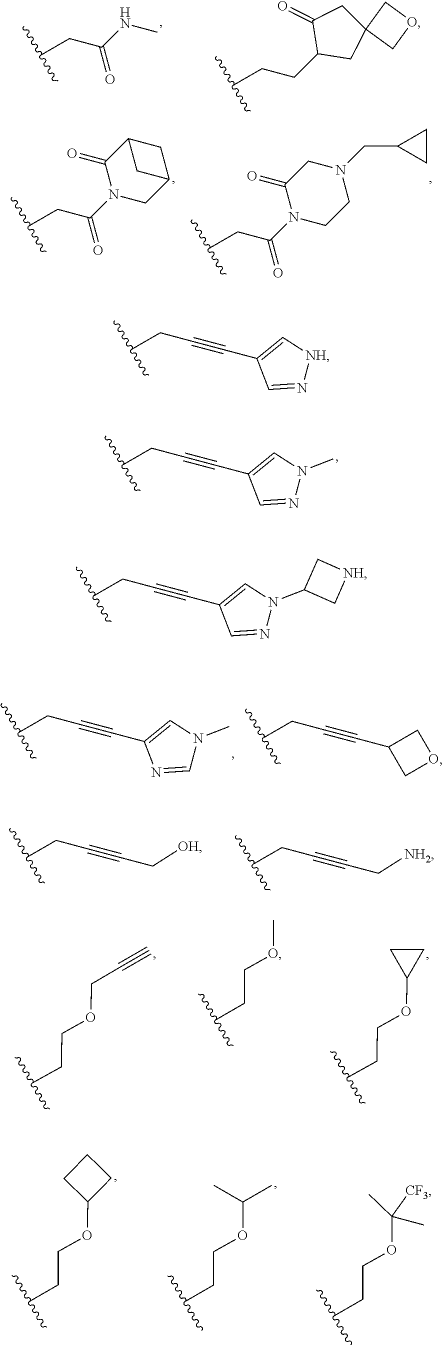

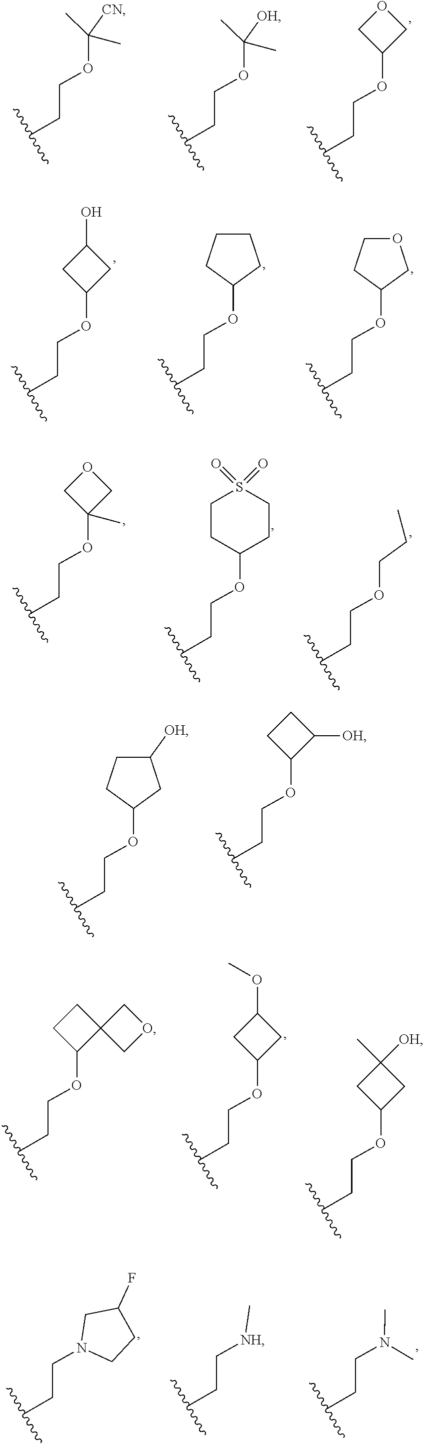

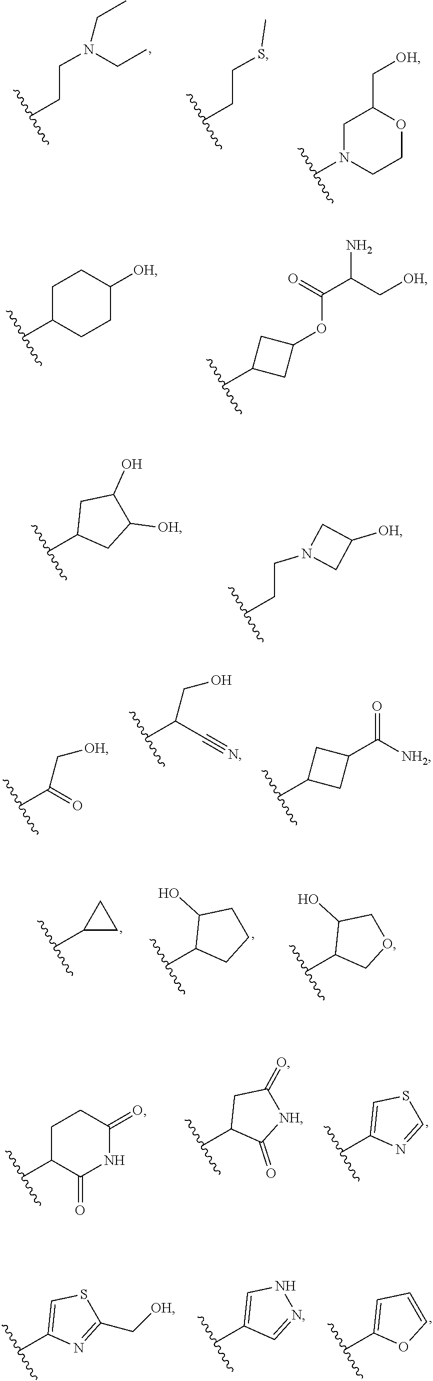

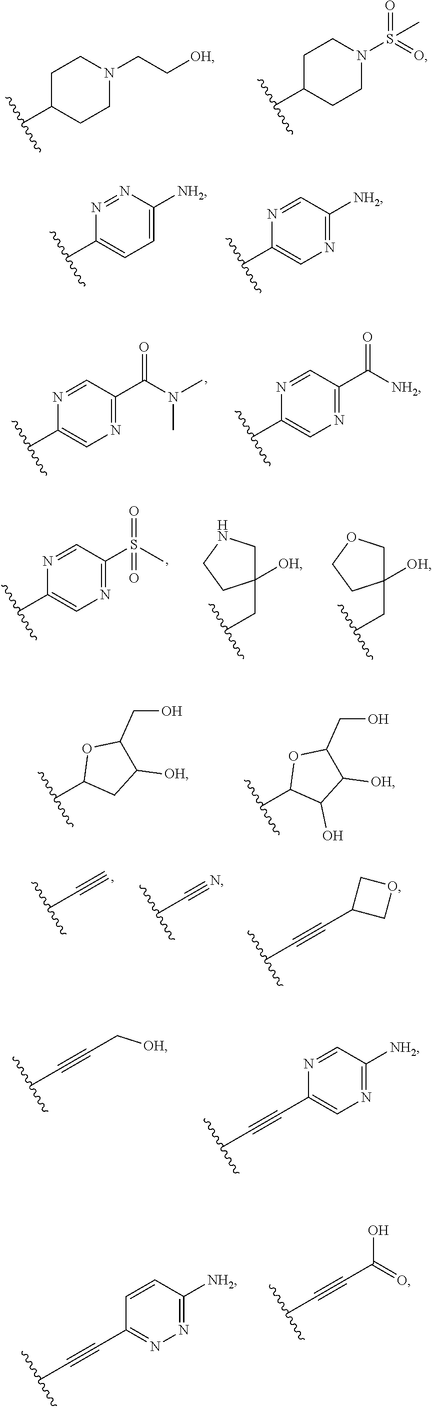

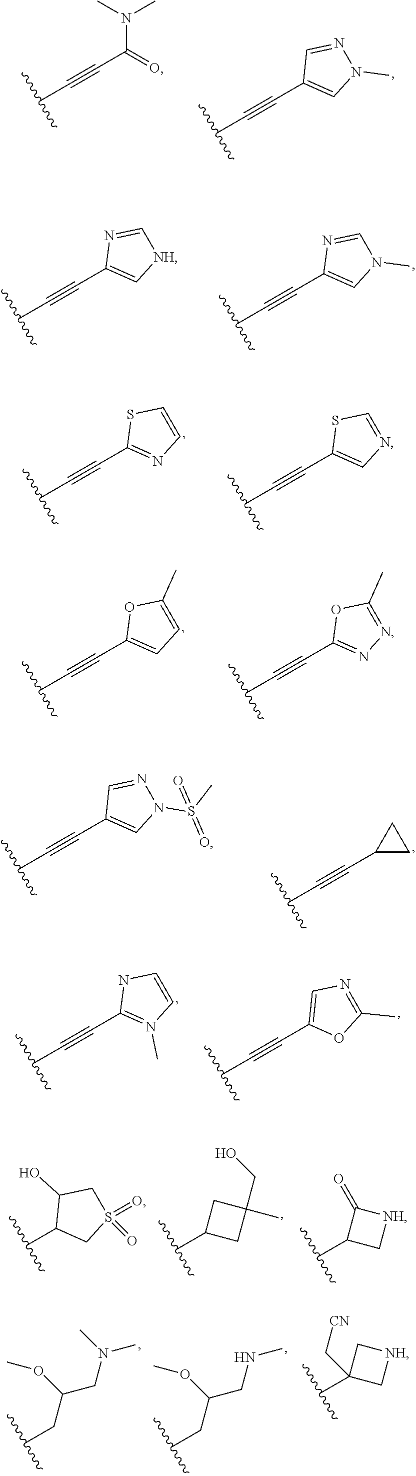

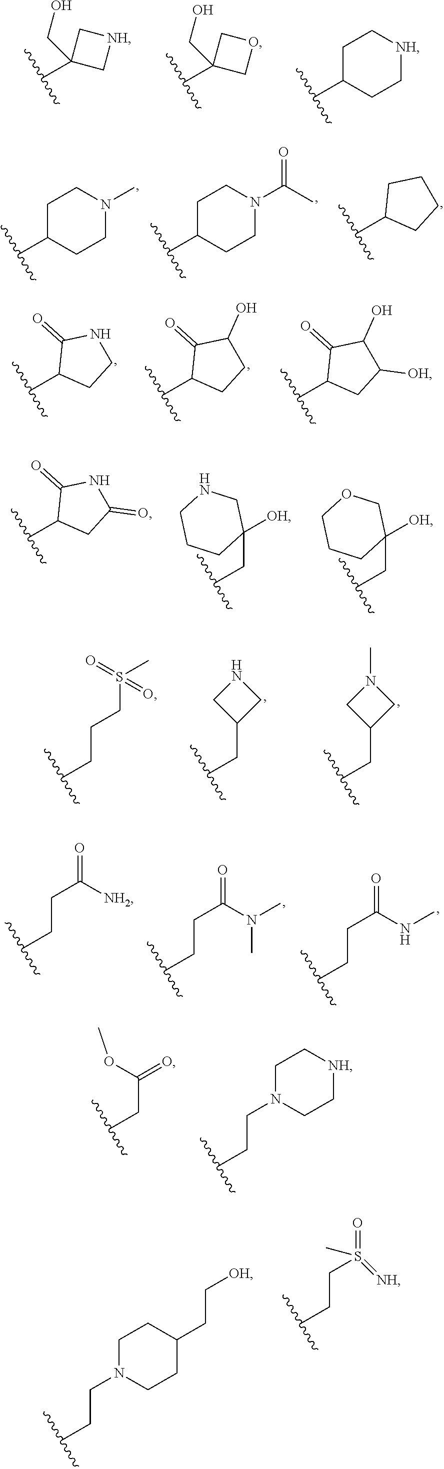

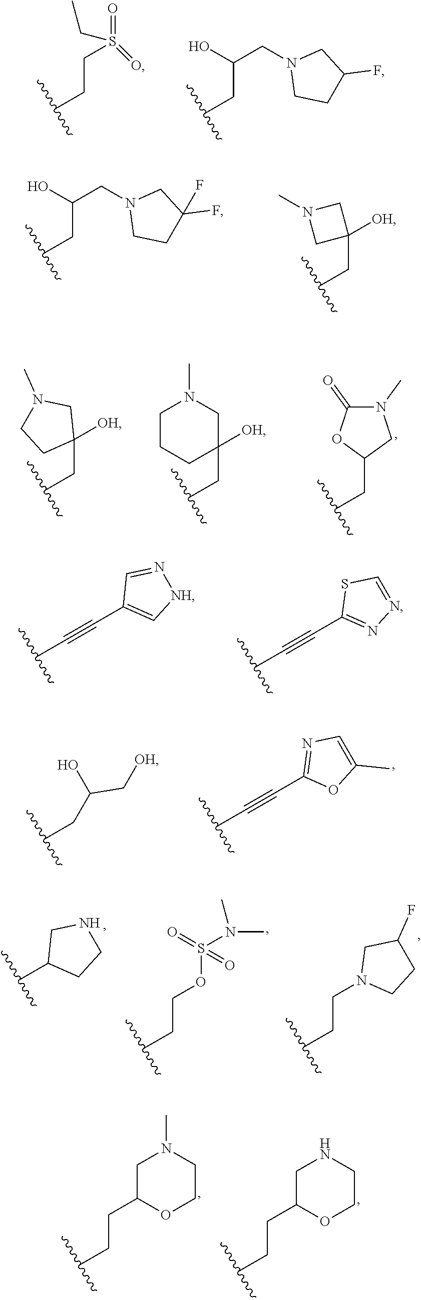

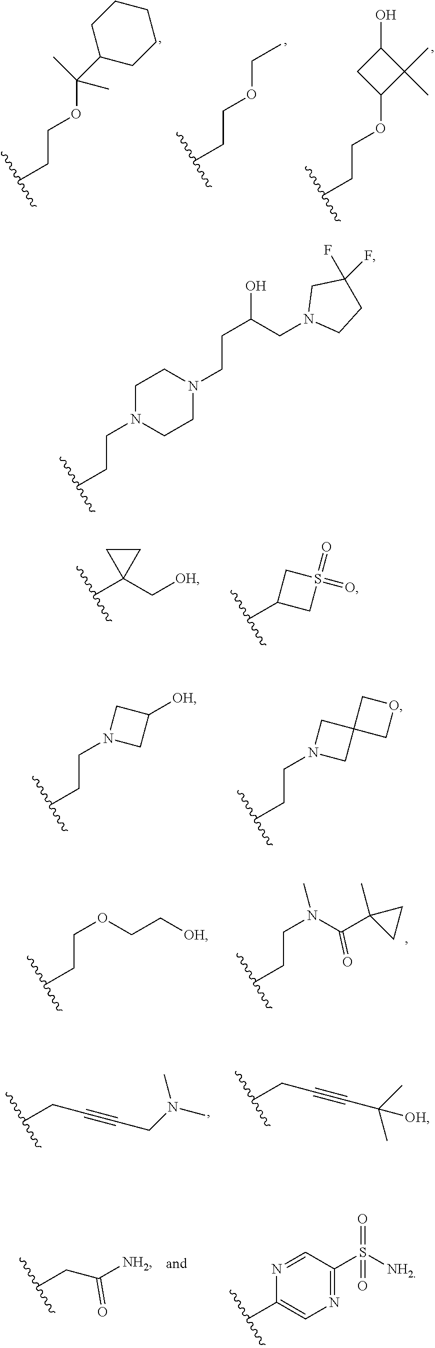

28. The compound of claim 1, wherein the compound of Formula (I) is represented by Formula (Ia), Formula (Ib), Formula (Ic), Formula (Ic′), Formula (Id), Formula (Id′), Formula (Ie), or Formula (Ie′):

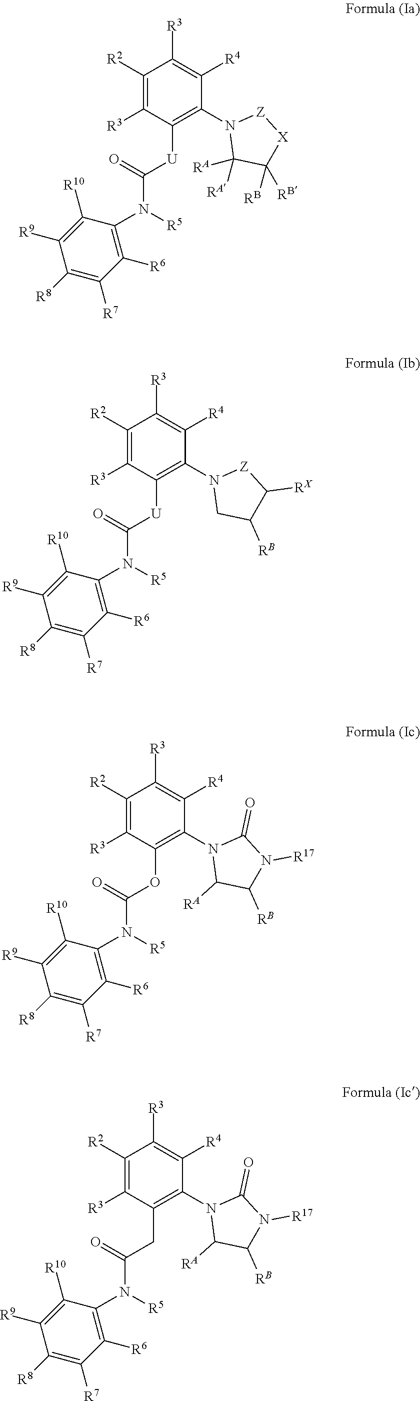

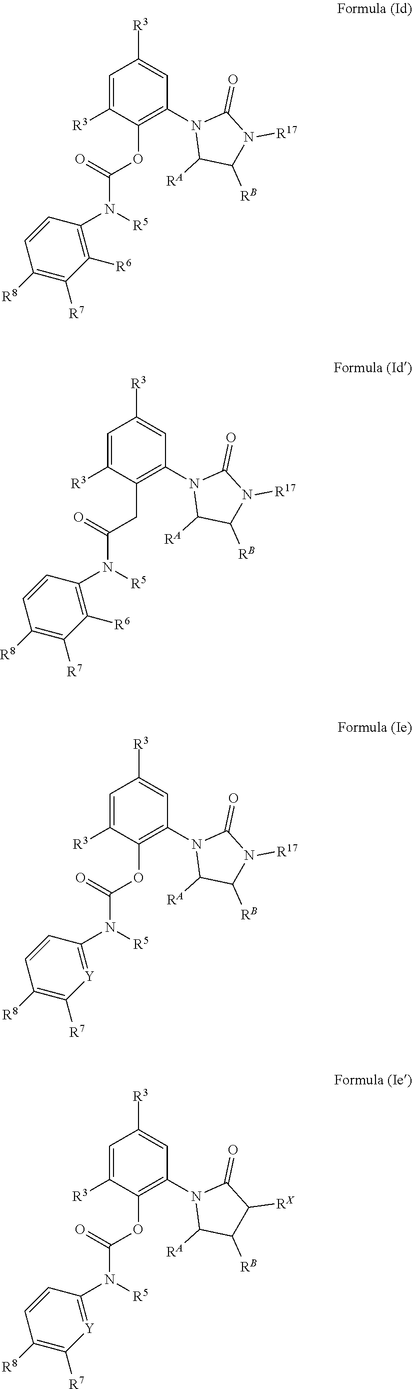

29.-32. (canceled)

33. A method of treating cancer in a subject in need thereof, the method comprising administering to the subject a compound of Formula (I) or a tautomeric or a stereochemically isomeric form, a pharmaceutically acceptable salt or a solvate thereof: 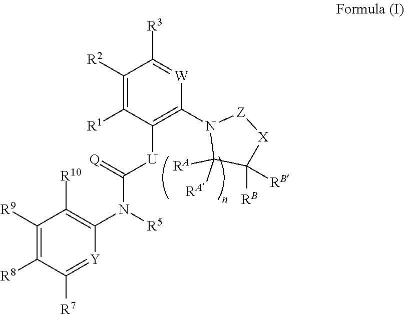
wherein:
U represents CH₂, O, S, or NR^{U};
W represents C(R⁴) or N;
Y represents C(R⁶) or N;
Q represents O or S;
R¹, R², R³, R⁴, R⁶, R⁷, R⁸, R⁹, and R¹⁰ independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, NR^{X}R^{Y}, and combinations thereof, wherein two adjacent groups R¹ to R⁴ or R⁶ to R¹⁰ optionally join to form a 5- to 7-membered saturated or unsaturated ring optionally containing one or more heteroatoms selected from O, N or S;
R⁵ and R^{U} independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof,
Z represents CR^{Z}R^{Z′}, C═S, or C═O;
X represents C(R¹⁵)(R¹⁶), N(R¹⁷) or O;
R¹⁵, R¹⁶, and R¹⁷ independently represent hydrogen, deuterium, C₁₋₆ alkyl, haloC₁₋₆ alkyl, —OR^{15a}, —SR^{15a}, nitrile, —COC₁₋₆ alkyl, —COOC₁₋₆ alkyl, hydroxy, C₁₋₆ alkoxy, C₁₋₆ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof; wherein R¹⁵, R¹⁶, and R¹⁷ may further comprise one or more divalent linkers L selected from the group consisting alkylene, cycloalkylene, heteroalkylene, heterocycloalkylene, alkenylene, alkynylene, arylene, heteroarylene, silyl, amine, amide, ester, ether, carbonyl, carbamate, sulfamate, sulfonic ester, sulfoximine, sulfonamide, thioether, thioester, disulfide, hydrazine, urea, thiourea, phosphate, phosphonate ester, poly(alkyl ether), heteroatom, and combinations thereof, wherein R¹⁵ and R¹⁶ may be taken together to form a ring;
n is 0, 1, or 2; wherein when n is 0, N is directly bonded to the carbon having R^{B} and R^{B′} bound thereto;
each R^{A}, R^{A′}, R^{Z}, and R^{Z′} independently represents hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof;
R^{B} and R^{B′} independently represents hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof, or R^{B} and the carbon to which it is bound together form a carbonyl group and R^{B′} is not present;
R^{15a} and R^{15b} independently represent hydrogen, deuterium, or C₁₋₆ alkyl; wherein two groups R^{15a} and R^{15b}, or two groups R^{15b}, may join together to form a 5 to 7 membered saturated ring system which may be optionally substituted by one or more C₁₋₆ alkyl groups;
R^{V}, R^{W}, R^{X} and R^{Y} independently represent hydrogen, deuterium, C₁₋₆ alkyl, haloC₁₋₆ alkyl, C₃₋₈ cycloalkyl, —COC₁₋₆ alkyl or heterocyclyl; wherein said alkyl groups may be optionally substituted with or more deuterium, hydroxy, amino or sulfone groups; and said heterocyclyl ring may be optionally substituted by one or more deuterium, oxo, hydroxy, C₁₋₆ alkanol or —COC₁₋₆ alkyl groups.

34. The method of claim 33, further comprising the step of administering to the subject one or more PARP inhibitors, one or more topoisomerase inhibitors, one or more DNA damaging agents, one or more platinum agents, one or more DNA damage response inhibitors, one or more ATR inhibitors, one or more WEE1 inhibitors, one or more DNA-PK inhibitors, one or more ATM inhibitors, anti-cancer radiotherapy, or proton beam therapy.

35. A method of inhibiting the activity of DNA polymerase theta (Polq), the method comprising the step of contacting Polq with a compound of Formula (I) or a tautomeric or a stereochemically isomeric form, a pharmaceutically acceptable salt or a solvate thereof: 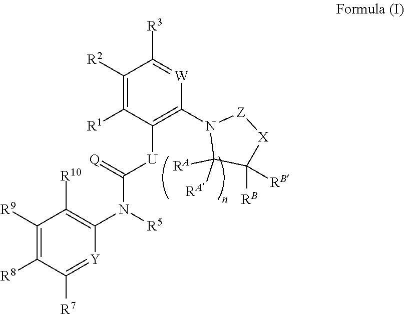
wherein:
U represents CH₂, O, S, or NR^{U};
W represents C(R⁴) or N;
Y represents C(R⁶) or N;
Q represents O or S;
R¹, R², R³, R⁴, R⁶, R⁷, R⁸, R⁹, and R¹⁰ independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, NR^{X}R^{Y}, and combinations thereof, wherein two adjacent groups R¹ to R⁴ or R⁶ to R¹⁰ optionally join to form a 5- to 7-membered saturated or unsaturated ring optionally containing one or more heteroatoms selected from O, N or S;
R⁵ and R^{U} independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof;
Z represents CR^{Z}R^{Z′}, C═S, or C═O;
X represents C(R¹⁵)(R¹⁶), N(R¹⁷) or O;
R¹⁵, R¹⁶, and R¹⁷ independently represent hydrogen, deuterium, C₁₋₆ alkyl, haloC₁₋₆ alkyl, —OR^{15a}, —SR^{15a}, nitrile, —COC₁₋₆ alkyl, —COOC₁₋₆ alkyl, hydroxy, C₁₋₆ alkoxy, C₁₋₆ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof; wherein R¹⁵, R¹⁶, and R¹⁷ may further comprise one or more divalent linkers L selected from the group consisting alkylene, cycloalkylene, heteroalkylene, heterocycloalkylene, alkenylene, alkynylene, arylene, heteroarylene, silyl, amine, amide, ester, ether, carbonyl, carbamate, sulfamate, sulfonic ester, sulfoximine, sulfonamide, thioether, thioester, disulfide, hydrazine, urea, thiourea, phosphate, phosphonate ester, poly(alkyl ether), heteroatom, and combinations thereof, wherein R¹⁵ and R¹⁶ may be taken together to form a ring;
n is 0, 1, or 2; wherein when n is 0, N is directly bonded to the carbon having R^{B} and R^{B′} bound thereto;
each R^{A}, R^{A′}, R^{Z}, and R^{Z′} independently represents hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof,
R^{B} and R^{B′} independently represents hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof, or R^{B} and the carbon to which it is bound together form a carbonyl group and R^{B′} is not present;
R^{15a} and R^{15b} independently represent hydrogen, deuterium, or C₁₋₆ alkyl; wherein two groups R^{15a} and R^{15b}, or two groups R^{15b}, may join together to form a 5 to 7 membered saturated ring system which may be optionally substituted by one or more C₁₋₆ alkyl groups;
R^{V}, R^{W}, R^{X} and R^{Y} independently represent hydrogen, deuterium, C₁₋₆ alkyl, haloC₁₋₆ alkyl, C₃₋₈ cycloalkyl, —COC₁₋₆ alkyl or heterocyclyl; wherein said alkyl groups may be optionally substituted with or more deuterium, hydroxy, amino or sulfone groups; and said heterocyclyl ring may be optionally substituted by one or more deuterium, oxo, hydroxy, C₁₋₆ alkanol or —COC₁₋₆ alkyl groups.

36. The method of claim 35, further comprising the step of administering to the subject one or more PARP inhibitors, one or more topoisomerase inhibitors, one or more DNA damaging agents, one or more platinum agents, one or more DNA damage response inhibitors, one or more ATR inhibitors, one or more WEE1 inhibitors, one or more DNA-PK inhibitors, one or more ATM inhibitors, anti-cancer radiotherapy, or proton beam therapy.


SEQUENCE LISTING (5 sequences)

SEQ ID NO: 1 - protein, 799 aa, Homo sapiens
GFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 2 - protein, 2590 aa, Homo sapiens
MNLLRRSGKRRRSESGSDSFSGSGGDSSASPQFLSGSVLSPPPGLGRCLKAAAAGECKPTVPDYERDKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEMGVQWNPCALLHSSTCSLTHSESEVKEHTFISQTKSSYKKLTSKNKSNTIFSDSYIKHSPNIVQDLNKSREHTSSFNCNFQNGNQEHQTCSIFRARKRASLDINKEKPGASQNEGKTSDKKVVQTFSQKTKKAPLNFNSEKMSRSFRSWKRRKHLKRSRDSSPLKDSGACRIHLQGQTLSNPSLCEDPFTLDEKKTEFRNSGPFAKNVSLSGKEKDNKTSFPLQIKQNCSWNITLTNDNFVEHIVTGSQSKNVTCQATSVVSEKGRGVAVEAEKINEVLIQNGSKNQNVYMKHHDIHPINQYLRKQSHEQTSTITKQKNIIERQMPCEAVSSYINRDSNVTINCERIKLNTEENKPSHFQALGDDISRTVIPSEVLPSAGAFSKSEGQHENFLNISRLQEKTGTYTTNKTKNNHVSDLGLVLCDFEDSFYLDTQSEKIIQQMATENAKLGAKDTNLAAGIMQKSLVQQNSMNSFQKECHIPFPAEQHPLGATKIDHLDLKTVGTMKQSSDSHGVDILTPESPIFHSPILLEENGLFLKKNEVSVTDSQLNSFLQGYQTQETVKPVILLIPQKRTPTGVEGECLPVPETSLNMSDSLLFDSFSDDYLVKEQLPDMQMKEPLPSEVTSNHFSDSLCLQEDLIKKSNVNENQDTHQQLTCSNDESIIFSEMDSVQMVEALDNVDIFPVQEKNHTVVSPRALELSDPVLDEHHQGDQDGGDQDERAEKSKLTGTRQNHSFIWSGASFDLSPGLQRILDKVSSPLENEKLKSMTINFSSLNRKNTELNEEQEVISNLETKQVQGISFSSNNEVKSKIEMLENNANHDETSSLLPRKESNIVDDNGLIPPTPIPTSASKLTFPGILETPVNPWKTNNVLQPGESYLFGSPSDIKNHDLSPGSRNGFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 3 - DNA, 37 bp, synthetic construct
  source 1..37: mol_type = other DNA
  modified_base 1: mod_base = OTHER; note = nucleotide modified with a 5' Cy5 cyanine fluorophore tag
cactgtgagcttagtcacatttcatcatgcaggacag

SEQ ID NO: 4 - DNA, 14 bp, synthetic construct
  source 1..14: mol_type = other DNA
  modified_base 14: mod_base = OTHER; note = nucleotide modified with a 3' Iowa Black(R) RQ Sp quencher
ctaagctcacagtg

SEQ ID NO: 5 - DNA, 15 bp, synthetic construct
  source 1..15: mol_type = other DNA
ctgtcctgcatgatg